(19) United States
(12) Patent Application Publication
(10) Pub. No.: US 2007/0082912 A1
(43) Pub. Date: Apr. 12, 2007

(54) Pyrrole compounds for the treatment of prostaglandine mediated diseases

(76) Inventors: Gerard Martin Paul Giblin, RESEARCH TRIANGLE PARK, NC (US); Adrian Hall, Welwyn (GB); Mark Patrick Healy, Welwyn (GB); Xiao Qing Lewell, Stevenage (GB); Neil Derek Miller, Stevenage (GB); Riccardo Novelli, Welwyn (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/516,230

(22) PCT Filed: May 30, 2003
(86) PCT No.: PCT/EP03/05790

§ 371 Date: Aug. 22, 2006

(30) Foreign Application Priority Data
May 31, 2002 (GB) 0212785.0

(51) Int. Cl. A61K 31/501; A61K 31/496; A61K 31/497; A61K 31/4439; C07D 403/02

(52) U.S. Cl. 514/252.05; 514/255.05; 514/256; 544/238; 544/333; 544/405; 514/343; 546/278.4

(57) ABSTRACT

Compounds of formula (I) or a pharmaceutically acceptable derivative thereof: 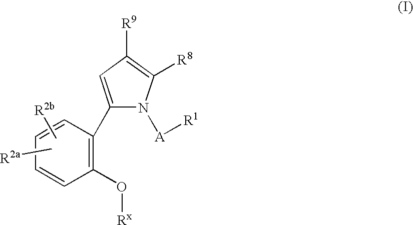
wherein A, R¹, R^{2a}, R^{2b}, R^{x}, R⁸, and R⁹ are as defined in the specification, a process for the preparation of such compounds, pharmaceutical compositions comprising such compounds and the use of such compounds in medicine.

This invention relates to pyrrole compounds, to processes for their preparation, to pharmaceutical compositions containing them and to their use in medicine, in particular their use in the treatment of prostaglandin mediated diseases.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology,1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-871 1) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289R-294), studies suggest that PGE₂-induced hyperthermia in the rat is mediated predominantly through the EP₁ receptor. WO 96/06822 (Mar. 7, 1996), WO 96/11902 (Apr. 25, 1996), EP 752421-A1 (Jan. 8, 1997) and WO 01/19814 (22 Mar. 2001) disclose compounds as being useful in the treatment of prostaglandin mediated diseases.

Accordingly the present invention provides compounds of formula (I): 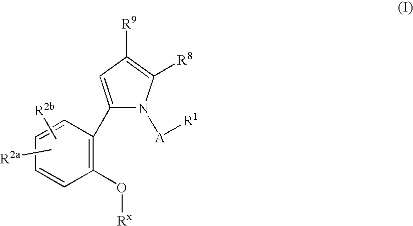
wherein:
A represents an optionally substituted aryl group, or an optionally substituted 5- or 6-membered heterocyclyl ring, or an optionally substituted bicyclic heterocyclyl group;
R¹ represents CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms may optionally be replaced by a group independently selected from NR⁴, O and SOₙ, wherein n is 0, 1 or 2: or R^{x} may be optionally substituted CQ₂-heterocyclyl, optionally substituted CQ₂-bicyclic heterocyclyl or optionally substituted CQ₂-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ₂aryl, optionally substituted CQ₂heteroaryl or COR⁷;
R⁷ represents hydrogen, optionally substituted alkyl, optionally substituted heteroaryl or optionally substituted aryl;
R⁸ represents hydrogen, CF₃, or alkyl;
R⁹ represents hydrogen, CF₃ or alkyl;
Q is independently selected from hydrogen and CH₃;
wherein when A is a 6-membered ring the R¹ substituent and pyrrole ring are attached to carbon atoms 1,2-, 1,3- or 1,4- relative to each other, and when A is a five-membered ring or bicyclic heterocyclyl group the R¹ substituent and pyrrole ring are attached to substitutable carbon atoms 1,2- or 1,3- relative to each other;
or a derivative thereof.

When A is a six membered ring, preferably the R¹ substituent is attached to A in the 3 or 4 position relative to the bond attaching A to the pyrrole ring. When R¹ is CO₂H, preferably the substituent is attached to A in the 3-position relative to the bond attaching A to the pyrrole ring.

Examples of A include phenyl, naphthyl, indolyl, pyridyl, pyridazinyl, pyrazinyl or pyrimidinyl, all of which may be optionally substituted. Particular examples include optionally substituted phenyl, optionally substituted pyridyl, indolyl or naphthyl. Preferably A is pyridyl or an optionally substituted phenyl; most preferably A is optionally substituted phenyl. In an alternative embodiment A is preferably pyridyl, more preferably A is 2,6 disubstituted pyridyl. In an alternative aspect A is selected from phenyl, pyridyl, pyridazinyl, pyrazinyl and pyrimidinyl, all of which may be optionally substituted.

Examples of optional substituents for A when a phenyl group include up to four substituents, preferably up to three substituents, more preferably up to two substituents independently selected from halogen, C₁₋₄haloalkyl, C₁₋₄haloalkoxy, NR⁴R⁵, NR⁶COC₁₋₆alkyl, NR⁵SO₂C₁₋₆alkyl, OR⁵, C₁₋₆alkyl, SO₂C₁₋₆alkyl, NR⁵COCH₂OC₁₋₆alkyl, optionally substituted NR⁵COCH₂Oaryl, and optionally substituted NR⁵COCH₂heteroaryl, wherein R⁴ and R⁵ are each independently selected from hydrogen and C₁₋₄alkyl; and NR¹⁰R¹¹ wherein R¹⁰ and R¹¹ together with the nitrogen atom to which they are attached form an optionally substituted 5- or 6-membered aliphatic heterocyclic ring wherein one of the ring carbons may be optionally replaced by another heteroatom selected from O, and SOₙ wherein n is 0, 1 or 2.

Examples of substituents for the 5- or 6-membered aliphatic heterocyclic ring include oxo.

Preferably optional substituents for A when a phenyl group are selected from halogen, CF₃, OCHF₂, NR⁴R⁵, NR⁵COC₁₋₆alkyl, NR⁵SO₂C₁₋₆alkyl, OR⁵, C₁₋₆alkyl, SO₂C₁₋₆alkyl, NR⁵COCH₂OC₁₋₆alkyl, NR⁵COCH₂thienyl, morpholinyl, pyrrolidinyl, 2-oxopyrrolidinyl, 2-oxopiperidinyl and 1,1-dioxo-1I⁶-isothiazolidinyl wherein R⁴ and R⁵ are each selected from hydrogen and C₁₋₄alkyl.

Optional substituents for A when a 5- or 6-membered heterocycyl group include NH₂. When A is pyridyl it may be substituted on the ring nitrogen by an oxygen to give a pyridine N-oxide.

Examples of R¹ include CO₂H, CN, CONR⁴R⁵, optionally substituted CONR⁵SO₂aryl, optionally substituted CONR⁵SO₂heteroaryl, optionally substituted CONR⁵aryl, optionally substituted CONR⁵heteroaryl e.g. CONR⁵tetrazolyl and CONR⁵pyridyl, CONR⁵SO₂C₁₋₆alkyl, optionally substituted CONR⁵SO₂heteroaryl e.g. CONR⁵SO₂-3,5-dimethylisoxazolyl, optionally substituted CONR⁵CQ₂aryl, optionally substituted CONR⁵CQ₂heteroaryl, optionally substituted C₁₋₆alkyl e.g. CF₃C(OH)CF₃, SO₂C₁₋₆alkyl, SO₂NR⁴R⁵, optionally substituted SO₂NR⁵COaryl, optionally substituted SO₂NR⁵COheteroaryl e.g SO₂NR⁵CO-3,5-dimethylisoxazolyl, SO₂NR⁵COC₁₋₆alkyl, optionally substituted SO₂NR⁵CQ₂aryl, optionally substituted SO₂NR⁵CQ₂heteroaryl; CO₁₋₆alkyl, 2H-tetrazol-5-yl-methyl, optionally substituted bicyclic heterocycyl e.g. benzimidazolyl, or optionally substituted heterocyclyl e.g. tetrazolyl, imidazolyl, methyloxadiazolyl and oxadiazolyl; wherein R⁴ and R⁵ are each selected from hydrogen and C₁₋₄alkyl, and Q is selected from hydrogen and CH₃.

When R¹ is optionally substituted heterocyclyl it is preferably tetrazolyl.

Preferably R¹ represents CONHCQ₂aryl, CONHCQ₂heteroaryl, CONHSO₂aryl, CONHSO₂heteroaryl, SO₂NHCOaryl, SO₂NHCOheteroaryl all of which may be optionally substituted, CO₂H, tetrazolyl or SO₂CH₃. More preferably R¹ represents CONHCHQphenyl, CONHSO₂phenyl, SO₂NHCOphenyl, all of which may be optionally substituted, CO₂H, tetrazolyl or SO₂CH₃. Most preferably R¹ represents CO₂H.

Preferably aryl is optionally substituted phenyl.

Preferably Q is hydrogen.

When R^{x} represents an optionally substituted alkyl this group is preferably C₁₋₈alkyl, more preferably the alkyl group is CH₂C₅₋₆cycloalkyl wherein 1 or 2 of the ring carbon atoms may optionally be replaced by a group independently selected from NR⁴, O or SOₙ, wherein n is 0, 1 or 2 and R⁴ is selected from hydrogen and C₁₋₄alkyl.

Examples of R^{x} include CH₂CH(CH₃)₂, CH₂cyclohexyl, CH₂tetrahydrofuranyl, CH₂ tetrahydropyranyl, optionally substituted CH₂-heterocyclyl e.g. CH₂methylisoxazolyl, optionally substituted CH₂-bicyclic heterocyclyl e.g. CH₂benzofurazanyl, optionally substituted CH₂naphthyl or optionally substituted CH₂-phenyl. Examples of substituents for CH₂phenyl and CH₂naphthyl include up to 4 substituents independently selected from halogen, optionally substituted C₁₋₆alkyl, C₁₋₄haloalkyl, C₁₋₆haloalkoxy, optionally substituted phenyl, and optionally substituted OC₁₋₆alkyl. Particular examples include up to three substituents independently selected from halogen, C₁₋₄alkyl, CF₃, phenyl, OC₁₋₄alkyl and OCHF₂. Preferred substituents include up to three substituents independently selected from chloro, bromo and fluoro.

In a preferred aspect R^{x} is optionally substituted CH₂-phenyl.

Preferably R^{2a} is hydrogen.

Preferably R^{2b}represents hydrogen, fluoro, chloro, bromo, optionally substituted C₁₋₄alkyl, e.g. CF₃, and CH₃, phenyl or SO₂C₁₋₄alkyl, e.g. SO₂CH₃. More preferably R^{2b} represents hydrogen, fluoro, chloro, bromo, or CF₃.

Preferably R^{2b} is positioned on the phenyl ring meta to the pyrrole group and pare to the oxy substituent.

R⁴ is preferably hydrogen or C₁₋₆alkyl, more preferably hydrogen or C₁₋₄alkyl.

R⁵ is preferably hydrogen or C₁₋₆alkyl, more preferably hydrogen or C₁₋₄alkyl.

R⁸ preferably represents CH₃.

R⁹ preferably represents hydrogen.

In an alternative aspect:

A represents an optionally substituted phenyl, or a 5 or 6 membered heterocyclyl group;

R¹ represents CO₂R⁴, CONR⁵R⁶, CH₂CO₂R⁴, optionally substituted C₁₋₆alkyl, optionally substituted C₁₋₆alkenyl, SO₂C₁₋₆alkyl, SO₂NR⁵R⁶, NR⁵CONR⁵R⁶, tetrazolyl or CONR⁵R⁶;

R^{2a} and R^{2b} independently represent hydrogen, halo, CF₃ optionally substituted C₁₋₆alkyl, CN, SO₂R⁵, NO₂, optionally substituted aryl, CONR⁵R⁶ or optionally substituted heteroaryl;

R^{x} represents optionally substituted C₁₋₈alkyl or optionally substituted CH₂phenyl;

R⁴ represents hydrogen or an optionally substituted C₁₋₆alkyl;

R⁵ represents hydrogen or an optionally substituted C₁₋₆alkyl;

R⁶ represents hydrogen or an optionally substituted C₁₋₆alkyl, optionally substituted —SO₂aryl, optionally substituted SO₂heterocyclyl group, CN or COR⁷;

R⁷ represents hydrogen or an optionally substituted aryl;

R⁸ represents hydrogen, CF₃ or C₁₋₆alkyl;

R⁹ represents hydrogen, Cl, Br, I, CF₃ or C₁₋₆alkyl;

wherein R¹ is attached to the group A in the 3 position relative to the bond attaching A to the pyrrole ring;

or a pharmaceutically acceptable derivative thereof.

Preferred compounds of formula (I) are compounds of formula (Ia): 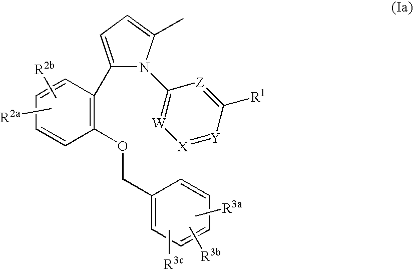
wherein:
R¹ is CO₂H;
R^{2a} and R^{2b} are independently selected from hydrogen, halo, phenyl, optionally substituted C₁₋₆alkyl e.g. C₁₋₄alkyl and CF₃, CN, SC₁₋₆alkyl, or SO₂C₁₋₆alkyl;
R^{3a}, R^{3b}, and R^{3c} are independently selected from hydrogen, halo, optionally substituted OC₁₋₆alkyl, e.g OCHF₂, phenyl or optionally substituted C₁₋₆alkyl e.g. CF₃;
W, X, Y and Z each represents CR¹² or N wherein at least two of W, X, Y or Z is CR¹²; and when each of W, X, Y, and Z is CR¹² then each R¹² is independently selected from hydrogen, halogen, C₁₋₄haloalkyl, C₁₋₄haloalkoxy, NR⁴R⁵, NR⁵COC₁₋₆alkyl, NR⁵SO₂C₁₋₆alkyl, OR⁵, C₁₋₆alkyl, SO₂C₁₋₆alkyl, NR⁵COCH₂OC₁₋₆alkyl, NR⁵COCH₂aryl, NR⁶COCH₂heteroaryl wherein R⁴ and R⁵ are each independently selected from hydrogen and C₁₋₄alkyl; and NR¹⁰R¹¹ wherein R¹⁰ and R¹¹ together with the nitrogen atom to which they are attached form an optionally substituted 5- or 6-membered aliphatic heterocyclic ring wherein one of the ring carbons may be optionally replaced by another heteroatom selected from O and SOₙ wherein n is 0, 1 or 2., and when at least one of W, X, Y and Z represents N then each R¹² is selected from hydrogen and NH₂;
or derivatives thereof.

In an alternative aspect of compounds of formula (Ia):

R¹ is CO₂R⁴;

R^{2a} and R^{2a} are independently selected from hydrogen, halo, optionally substituted C₁₋₆alkyl, CN or SO₂C₁₋₆alkyl;

R^{3a} and R^{3b} are independently selected from hydrogen, halo or an optionally substituted OC₁₋₆alkyl, or C₁₋₆alkyl;

R^{3c} is hydrogen;

R⁴ is hydrogen or an optionally substituted C₁₋₆alkyl;

W, X, Y and Z represents CH or N wherein at least one of W, X, Y or Z is CH;

or pharmaceutically acceptable derivatives thereof.

Preferably R^{2a} and R^{2b} are independently selected from hydrogen, chloro, fluoro, bromo and CF₃ More preferably R^{2a} is hydrogen and R^{2b} is selected from hydrogen, chloro, fluoro, bromo and CF₃.

Preferably R^{3a}, R^{3b} and R^{3c} independently selected from hydrogen, CF₃, chloro, fluoro and bromo.

Preferably one of W, X, Y and Z is selected from N and CR¹² and the remaining atoms are CR². More preferably Z is N and W, X and Y are CR¹². Most preferably Z is N and W, X and Y are CH. Alternatively W, X, Y and Z are each selected from CR¹²

Examples of compounds of formula (I) include:
3-{2-[2-(Benzyloxy)-phenyl]-5-methyl-pyrrol-1-yl}-benzoic acid;
3-{2-[2-(Benzyloxy)-5-chloro-phenyl]-5-methyl-pyrrol-1-yl}-benzoic acid;
3-{2-[5-Bromo-2-(benzyloxy)-phenyl]-5-methyl-pyrrol-1-yl}-benzoic acid;
3-{2-[5-Phenyl-2-(benzyloxy)-phenyl]-5-methyl-pyrrol-1-yl}-benzoic acid;
3-{2-[5-Chloro-2-(cyclohexylmethoxy)-phenyl]-5-methyl-pyrrol-1-yl}-benzoic acid;
4-{2-[5-Chloro-2-(benzyloxy)-phenyl]-5-methyl-1-pyrrol-1-yl}-methanesulfonyl benzene;
3-{2-[5-Chloro-2-(benzyloxy)-phenyl]-5-methyl-1-pyrrol-1-yl}-methanesulfonyl benzene;
3-{2-[5-Bromo-2-(4-methoxy-benzyloxy)-phenyl]-5-methyl-pyrrol-1-yl}-benzoic acid;
3-{2-[5-Bromo-2-(3,4-dichloro-benzyloxy)-phenyl]-5-methyl-pyrrol-1-yl}-benzoic acid;
3-{2-[5-Bromo-2-(2-chloro-4-fluoro-benzyloxy)-phenyl]-5-methyl-pyrrol-1-yl}-benzoic acid;
3-{2-[5-Bromo-2-(4-fluoro-benzyloxy)-phenyl]-5-methyl-pyrrol-1-yl}-benzoic acid;
3-{2-[5-Bromo-2-(2,4-difluoro-benzyloxy)-phenyl]-5-methyl-pyrrol-1-yl}-benzoic acid;
3-{2-[5-Bromo-2-(4-chloro-benzyloxy)-phenyl]-5-methyl-pyrrol-1-yl}-benzoic acid;
3-{2-[2-(4-Methoxy-benzyloxy)-phenyl]-pyrrol-1-yl}-benzoic acid;
3-{2-[2-(3,4-Dichloro-benzyloxy)-phenyl]-5-methyl-pyrrol-1-yl}-benzoic acid;
3-{2-[2-(2-Chloro-4-fluoro-benzyloxy)-phenyl]-5-methyl-pyrrol-1-yl}-benzoic acid;
3-{2-[2-(4-Fluoro-benzyloxy)-phenyl]-5-methyl-pyrrol-1-yl}-benzoic acid;
3-{2-[2-(2,4-Difluoro-benzyloxy)-phenyl]-5-methyl-pyrrol-1-yl}-benzoic acid;
3-{2-[2-(4-Chloro-benzyloxy)-phenyl]-5-methyl-pyrrol-1-yl}-benzoic acid;
3-{2-[5-Chloro-2-(4methoxy-benzyloxy)-phenyl]-5-methyl-pyrrol-1-yl}-benzoic acid;
3-{2-[5-Chloro-2-(4-chloro-benzyloxy)-phenyl]-5-methyl-pyrrol-1-yl}-benzoic acid;
3-{2-[5-Chloro-2-(3,4-dichloro-benzyloxy)-phenyl]-5-methyl-pyrrol-1-yl}-benzoic acid;
3-{2-[5-Chloro-2-(2-chloro-4-fluoro-benzyloxy)-phenyl]-5-methyl-pyrrol-1-yl}-benzoic acid;
3-{2-[5-Chloro-2-(4-fluoro-benzyloxy)-phenyl]-5-methyl-pyrrol-1-yl}-benzoic acid;
3-{2-[5-Chloro-2-(2,4-difluoro-benzyloxy)-phenyl]-5-methyl-pyrrol-1-yl}-benzoic acid;
5-{2-[2-(4-Methoxy-benzyloxy)-phenyl]-5-methyl-pyrrol-1-yl}-nicotinic acid;
5-{2-[2-(4-Chloro-benzyloxy)-phenyl]-5-methyl-pyrrol-1-yl}-nicotinic acid;
5-{2-[2-(3,4-Dichloro-benzyloxy)-phenyl]-5-methyl-pyrrol-1-yl}-nicotinic acid;
5-{2-[2-(4-Fluoro-benzyloxy)-phenyl]-5-methyl-pyrrol-1-yl}-nicotinic acid;
5-{2-[2-(2,4-Difluoro-benzyloxy)-phenyl]-5-methyl-pyrrol-1-yl}-nicotinic acid;
5-{2-[5-Chloro-2-(4-methoxy-benzyloxy)-phenyl]-5-methyl-pyrrol-1-yl}-nicotinic acid;
5-{2-[5-Chloro-2-(benzyloxy)-phenyl]-5-methyl-pyrrol-1-yl}-nicotinic acid;
   and derivatives thereof.

Preferred compounds include the compounds of Examples 11, 33, 41, 46, 49, 55, 60, 72, 76, 85, 88, 103, 106, 112, 122, 125, 150, 155, 157, 175, 176, 180, 183, 188, 191, 200, 207, 209, 211, 222, 225, 234, 235, 236, 237, 239, 240, 241, 245, 250, 254, 261, 262, 278, 283, 295, 306, 314, 316, 332, 338, 348, 353, 358, 356, 367, 376, 383, 385, 387, 388 and 392; and derivatives thereof.

More preferred compounds are the compounds of Examples 46, 60, 183, 222, 225, 234, 235, 236, 237, 239, 240, 241, 250, 254, 283 and 348; and derivatives thereof.

Preferably compounds are selective for EP₁ over EP₂, EP₃ and EP₄. More preferably the compounds are 100 fold selective, more preferably 1000 fold selective for EP₁.

The invention is described using the following definitions unless otherwise indicated.

Suitable derivatives are pharmaceutically acceptable derivatives.

The term “pharmaceutically acceptable derivative” means any pharmaceutically acceptable salt, ester, salt of such ester or solvate of the compounds of formula (I), or any other compound which upon administration to the recipient is capable of providing (directly or indirectly) a compound of formula (I) or an active metabolite or residue thereof.

It will be appreciated by those skilled in the art that the compounds of formula (I) may be modified to provide pharmaceutically acceptable derivatives thereof at any of the functional groups in the compounds, and that the compounds of formula (I) may be derivatised at more than one position.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2dimethylaminoethanol, ethanolamine, ethylenediamine, N-ethyl-morpholine, N-ethylpiperidine, glucamine, glucosamine, histidine, hydrabamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropyl amine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acid.

Preferred examples of pharmaceutically acceptable salts include those formed from maleic, fumaric, benzoic, ascorbic, pamoic, succinic, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The salts and/or solvates of the compounds of the formula (I) which are not pharmaceutically acceptable may be useful as intermediates in the preparation of pharmaceutically acceptable salts and/or solvates of compounds of formula (I) or the compounds of the formula (I) themselves, and as such form another aspect of the present invention.

The compounds of formula (I) may be prepared in crystalline or noncrystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine, more preferably fluorine, chlorine and bromine.

The term “alkyl” means a straight, branched or cyclic chain alkyl group or combinations thereof, for example a methyl, ethyl, n-propyl, i-propyl, n-butyl, s-butyl, t-butyl, pentyl, hexyl, 1,1-dimethylethyl, cyclopentyl or cyclohexyl or combinations thereof such as cyclohexylmethyl and cyclopentylmethyl. Unless otherwise defined, preferably “alkyl” is C₁₋₈alkyl, more preferably “alkyl” is C₁₋₆alkyl.

The term “alkoxy” means a straight, branched or cyclic chain alkyl group having an oxygen atom attached to the chain, for example a methoxy, ethoxy, n-propoxy, i-propoxy, n-butoxy, s-butoxy, t-butoxy group, pentoxy, hexyloxy group, cyclopentoxy or cyclohexyloxy group. Preferably “alkoxy” is C₁₋₆alkoxy.

The term “haloalkyl” means an alkyl group, including straight, branched or cyclic structures, of the indicated number of carbon atoms in which one or more hydrogen atoms have been replaced by halogen atoms, with up to complete substitution of all hydrogen atoms with halo groups. Preferably “haloalkyl” is C₁₋₆haloalkyl, more preferably C₁₋₄haloalkyl. C₁₋₆haloalkyl, for example, includes C₁₋₆fluoroalkyl, e.g. CF₃, CF₂CF₃, CHF₂, CH₂F and the like.

The term “haloalkoxy” means an alkoxy group, including straight, branched or cyclic structures, of the indicated number of carbon atoms in which one or more hydrogen atoms have been replaced by halogen atoms, with up to complete substitution of all hydrogen atoms with halo groups. Preferably “haloalkoxy” is C₁₋₆haloalkoxy, more preferably C₁₋₄haloalkoxy. C₁₋₆haloalkoxy, for example, includes C₁₋₆fluoroalkoxy e.g. OCF₃, OCHF₂, OCF₂CF₃ and the like.

The term “alkenyl” means linear or branched structures and combinations thereof, of the indicated number of carbon atoms, having at least one carbon-to-carbon double bond, wherein hydrogen may be replaced by an additional carbon to carbon double bond. Preferably “alkenyl” is C₂₋₆alkenyl. C₂₋₆alkenyl, for example, includes ethenyl, propenyl, 1-methylethenyl, butenyl and the like.

The term “aliphatic heterocyclyl” as a group or as part of a group means an aliphatic five or six membered ring which contains from 1 to 4 heteroatoms selected from nitrogen, oxygen or sulfur and unsubstituted or substituted by, for example, up to three substituents selected from halo, NH₂, C₁₋₄alkyl, C₁₋₄alkoxy, C₁₋₄haloalkyl, C₁₋₄haloalkoxy and oxo. Examples of 5-membered aliphatic heterocyclyl groups include pyrrolidinyl, dioxolanyl, imidazolidinyl, oxazolidinyl, isoxazolidinyl, thiazolidinyl, isothiazolidinyl, pyrazolidinyl, and tetrahydrofuranyl. Examples of 6-membered aliphatic heterocyclyl groups include morpholinyl, thiomorpholinyl, piperidinyl, dithianyl, piperazinyl and tetrahydropyranyl.

The term “heterocyclyl” as a group or as part of a group means an aromatic or non-aromatic five or six membered ring which contains from 1 to 4 heteroatoms selected from nitrogen, oxygen or sulfur and unsubstituted or substituted by, for example, up to three substituents selected from halo, oxo, NH₂, C₁₋₄alkyl, C₁₋₄alkoxy, C₁₋₄haloalkyl, and C₁₋₄haloalkoxy. Examples of 5-membered heterocyclyl groups include furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl or tetrazolyl. Examples of 6-membered heterocyclyl groups are pyridyl, pyridazinyl, pyrimidinyl, pyrazinyl, triazinyl or tetrazinyl.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is naphthyl or phenyl, more preferably phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents, selected from, for example, halo, NH₂, C₁₋₄alkyl, C₁₋₄alkoxy, C₁₋₄haloalkyl, and C₁₋₄haloalkoxy. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. A bicyclic heterocyclic group may be optionally substituted by one or more substituents, for example up to 3 or up to 2 substituents, selected from, for example, oxo, halo, NH₂, C₁₋₄alkyl, C₁₋₄alkoxy, C₁₋₄haloalkyl, and C₁₋₄haloalkoxy. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate be substituted by one or two substituents selected from hydrogen and C₁₋₈alkyl, preferably hydrogen and C₁₋₆alkyl, more preferably hydrogen.

Optional substituents for alkyl or alkenyl groups include OH, CO₂R⁴, NR⁴R⁵, (O), OC₁₋₆alkyl or halo, wherein R⁴ and R⁵ are selected from hydrogen and C₁₋₆alkyl. An alkyl or alkenyl group may be substituted by one or more optional substituents, for example up to 5, 4, 3, or 2 optional substituents.

Optional substituents for alkoxy groups include OH, CO₂R⁴, NR⁴R⁵, (O), OC₁₋₆alkyl or halo, wherein R⁴ and R⁵ are selected from hydrogen and C₁₋₆alkyl. An alkoxy group may be substituted by one or more optional substituents, for example up to 5, 4, 3, or 2 optional substituents.

Unless otherwise defined, examples of optional substituents for aryl, heteroaryl or heterocyclyl moieties as a group or part of a group are selected from optionally substituted C₁₋₆alkyl, optionally substituted C₁₋₆alkoxy and C₁₋₆haloalkyl, C₁₋₆haloalkoxy and halogen.

Compounds of formula (I) can be prepared as set forth in the following Schemes and in the Examples and references cited therein The following processes form another aspect of the present invention.

For example, compounds of formula (I) may be prepared by the general route below. 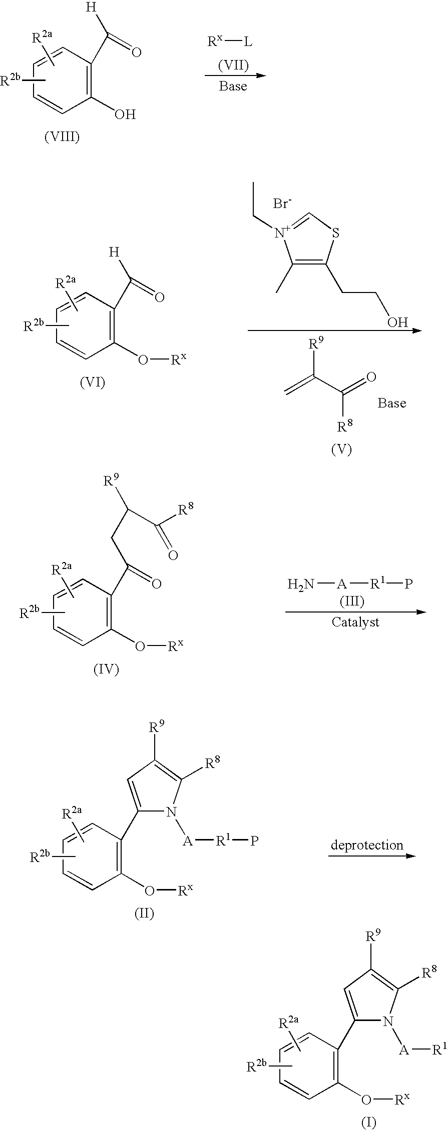
wherein L is a leaving group for example halo, e.g. bromo; P is an optional protecting group, for example methyl or ethyl esters; A, R⁸,R⁹,R^{2a}, R^{2b}, R¹ and R^{x} are as hereinbefore defined for compounds of formula (I).

When R¹ is CO₂H, a suitable protecting group P is an ester forming group such as C₁₋₄alkyl or optionally substituted benzyl. Suitable reaction conditions for the deprotection of a compound of formula (II) include hydrolysis effected by e.g. heating in ethanolic sodium hydroxide solution, or hydrogenation.

Suitable reaction conditions for the reaction of a compound of formula (IV) with a compound of formula (III) to give a pyrrole of formula (II) include heating with an acid catalyst e.g. p-toluenesulfonic acid in a solvent such as toluene. Reviews of pyrrole synthesis can be found in e.g. A. Triebs, Chem. Ber., 1957, 90, 79-84, E. Baltazzi et al., Chem. Rev., 1963, 63, 511, and R. A. Jones, Advances in Heterocyclyl Chemistry, 1970, 11, 383.

Suitable reaction conditions for the conversion of a compound of formula (VI) to a compound of formula (IV) include heating the compound of formula (VI) with a vinyl ketone of formula (V) in the presence of 3-ethyl-5-(2-hydroxyethyl)-4-methylthiazolium bromide and an organic base, for example triethylamine, in a solvent, for example ethanol.

Suitable reaction conditions for the preparation of a compound of formula (VI) include reacting a salicylaldehyde of formula (VIII) with a compound R^{x}-L of formula (VII) in N,N-dimethylformamide solution the presence of base, e.g. potassium carbonate.

Accordingly the present invention also provides a process for the preparation of a compound of formula (I) or a derivative thereof: 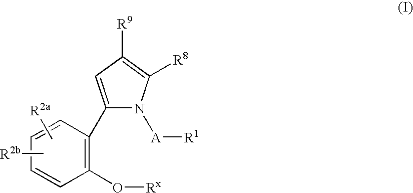
wherein:
A represents an optionally substituted aryl group, or an optionally substituted 5- or 6-membered heterocyclyl ring, or an optionally substituted bicyclic heterocyclyl group;
R¹ represents CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms may optionally be replaced by a group independently selected from NR⁴, O and SOₙ, wherein n is 0, 1 or 2: or R^{x} may be optionally substituted CQ₂-heterocyclyl, optionally substituted CQ₂-bicyclic heterocyclyl or optionally substituted CQ₂-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ₂aryl, optionally substituted CQ₂heteroaryl or COR⁷;
R⁷ represents hydrogen, optionally substituted alkyl, optionally substituted heteroaryl or optionally substituted aryl;
R⁸ represents hydrogen, CF₃, or alkyl;
R⁹ represents hydrogen, CF₃ or alkyl;
Q is independently selected from hydrogen and CH₃;
wherein when A is a 6-membered ring the R¹ substituent and pyrrole ring are attached to carbon atoms 1,2-, 1,3- or 1,4- relative to each other, and when A is a five-membered ring or bicyclic heterocyclyl group the R¹ substituent and pyrrole ring are attached to substitutable carbon atoms 1,2- or 1,3- relative to each other; comprising:
reacting a compound of formula (IV): 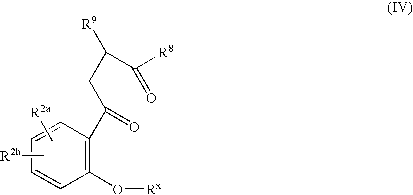
wherein R⁸, R⁹, R^{2a}, R^{2b}, and R^{x} are as hereinbefore defined above for a compound of formula (I);
with a compound of formula (III):
H₂N-A-R¹—P (III)
wherein A and R¹ are as hereinbefore defined above for a compound of formula (I), and P is an optional protecting group;
to give a compound of formula (II): 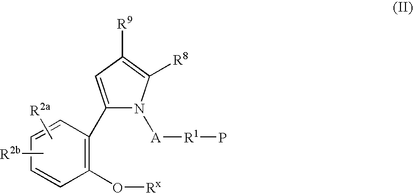
wherein P, A, R⁸, R⁹, R^{2a}, R^{2b}, R¹ and R^{x} are as hereinbefore defined;
and where required converting:
one group A to another group A, and/or
one group R^{2a} to another group R^{2a}; and/or
one group R^{2b} to another group R^{2b}; and/or
one group R^{x}to another group R^{x};
and where required carrying out the following optional steps in any order:
a) effecting deprotection; and/or
b) converting one group R¹ to another group R¹; and/or
c) forming a derivative of the compound of formula (I) so formed.

A group R¹ may be converted to another group R¹ by use of conventional organic transformations known to those skilled in the art. For example R¹═CO₂H may be converted to an amide, e.g. CONHCQ₂aryl or CONHCQ₂heteroaryl wherein Q is hydrogen or CH₃, by conventional methods for the preparation of amides as described in, for example, Richard Larock, Comprehensive Organic Transformations, 2nd edition, Wiley-VCH, ISBN 0-471-19031-4.

Compounds of formula (I) wherein A is a 2,6-disubstituted pyridine may also be prepared by the route described below: 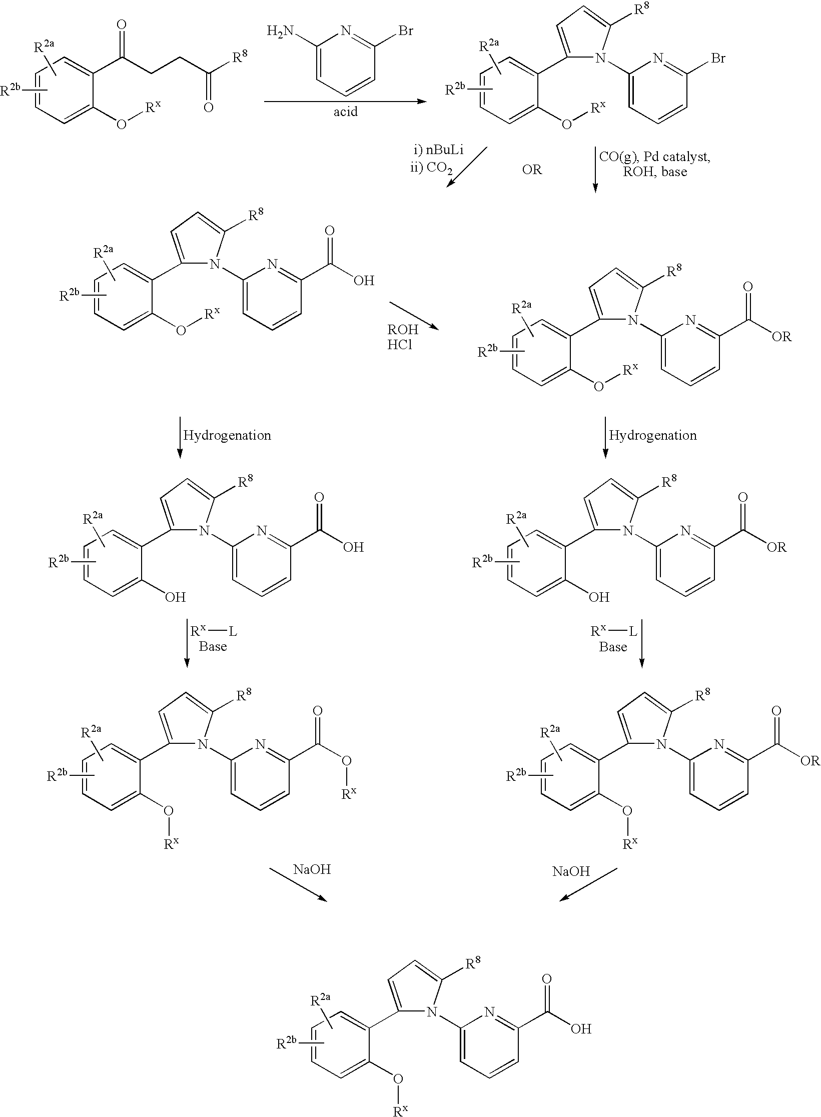
wherein L is a leaving group e.g. bromo, R is C₁₋₄alkyl, R^{2a} and R^{2b} are selected from hydrogen, halo and CF₃, and R⁸, and R^{x}e are as defined above for compounds of formula (I).

Compounds of formula (III), (V), (VII) and (VIII) and 3-ethyl-5-(2-hydroxyethyl)-4-methylthiazolium bromide are commercially available, or readily prepared by methods known to those skilled in the art.

Compounds of formula (III):
H₂N-A-R¹—P
wherein P is as defined above and R¹ and A are as hereinbefore defined for compounds of formula (I) are commercially available or may readily be prepared from commercially available materials according to known methods for preparing amines, e.g. using methods as described in the Examples. Methods for the preparation of amines are reviewed in The Amino Group, S. Patai (Ed), Interscience, New York 1968, and references cited therein. The preparation of amines is also described in Richard Larock, Comprehensive Organic Transformations, 2nd edition, pages 753 to 879, Wiley-VCH, ISBN 0-471-19031-4.

Intermediates of formula (V): 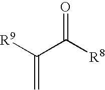
wherein R⁸ and R⁹ are as hereinbefore defined for compounds of formula (I) are commercially available or may be readily prepared according to known methods for the preparation of vinyl ketones. For example, F₃CCOCHCH₂═CH₂ may be prepared according to the method of M. Tordeux et al, J. Fluorine Chemistry, 1982, 20(3), 301-306.

Intermediates of formula (VII):
R^{x}-L
wherein L is as defined above and R^{x} is as defined for compounds of formula (I) are commercially available, or may be readily prepared by known transformations of commercially available compounds.

Intermediates of formula (VIII): 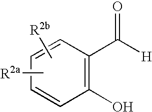
wherein R^{2a} and R^{2b} are as defined for compounds of formula (I) are commercially available, or may readily be prepared by methods known to those skilled in the art, for example from suitable commercially available starting materials using methods as described in the examples. The preparation of aldehydes is reviewed in The Chemistry of the Carbonyl Group, S. Patai (Ed), Interscience, New York, 1966, and references cited therein.

Certain substituents in any of the reaction intermediates and compounds of formula (I) may be converted to other substituents by conventional methods known to those skilled in the art. Examples of substituents which may be converted include one group R^{2a} to another group R^{2a}, one group R^{2b} to another group R^{2b}; one group R^{x}to another group R^{x}; and substituent on a group A to another substituent on a group A. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

For example, when R^{x} is p-methoxybenzyl, cleavage of the ether to give the phenol is carried out using, for example, using acid e.g. HCl/dioxane or using sodium methanethiolate. Conversion to another R^{x} group, for example a substituted benzyl group, may be effected by reaction of the phenol with a suitable substituted benzyl bromide. The skilled person will appreciate that conversion of the protecting group P to another protecting group P may also occur under the reaction conditions used. When R^{x} is benzyl, cleavage of the ether to give the phenol may be carried out by hydrogenation according to known methods e.g. H₂—Pd/C or NH₄CO₂H—Pd/C. The resulting phenol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and are therefore useful in treating EP₁ receptor mediated diseases.

In view of their ability to bind to the EP₁ receptor, the compounds of the invention may be useful in the treatment of the disorders that follow. Thus, the compounds of formula (I) may be useful as analgesics. For example they may be useful in the treatment of chronic articular pain (e.g. rheumatoid arthritis, osteoarthrits, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea. The compounds of the invention may also be useful in the treatment of visceral pain.

The compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) may also be useful in the treatment of fever.

The compounds of formula (I) may also be useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, polymyositis, gingivitis, myocardial ischemia, pyrexia, systemic lupus erythematosus, tendinitis, bursitis, and Sjogren's syndrome.

The compounds of formula (I) are also useful in the treatment of immunological diseases such as autoimmune diseases, immunological deficiency diseases or organ transplantation. The compounds of formula (I) are also effective in increasing the latency of HIV infection.

The compounds of formula (I) are also useful in the treatment of diseases of abnormal platelet function (e.g. occlusive vascular diseases).

The compounds of formula (I) are also useful for the preparation of a drug with diuretic action.

The compounds of formula (I) are also useful in the treatment of impotence or erectile dysfunction.

The compounds of formula (I) are also useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also useful for attenuating the hemodynamic side effects of non-steroidal anti-inflammatory drugs (NSAID's) and cyclooxygenase-2 (COX-2) inhibitors.

The compounds of formula (I) are also useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of formula (I) are also useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with aging, particularly Age Associated Memory Impairment.

The compounds of formula (I) are also useful in the treatment of neuroprotection and in the treatment of neurodegeneration following stroke, cardiac arrest, pulmonary bypass, traumatic brain injury, spinal cord injury or the like.

The compounds of formula (I) are also useful in the treatment of tinnitus.

The compounds of formula (I) are also useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) are also useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may, for example, be formulated for topical administration or administration by inhalation or, more preferably, for oral, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously).

The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ antagonists; EP₂ antagonists and EP₃ antagonists; cannabanoid receptor ligands; bradykinin receptor ligands and vanilloid receptor ligand. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/3831 1, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day, calculated as the free base, which may be administered as a single or divided dose, for example one to four times per day The dose range for adult human beings is generally from 8 to 2000 mg/day, such as from 20 to 1000 mg/day, preferably 35 to 200 mg/day, calculated as the free base.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

### EXAMPLES

Abbreviations

Definitions of abbreviations used herein: ethyl acetate (EtOAc), N,N-dimethylformamide (DMF), hexand (hex), dimethylsulfoxide (DMSO), dichloromethane (DCM), tetrahydrofuran (THF), 1-methyl-2-pyrrolidinone (NMP), 1-(3dimethylaminopropyl)-3-ethylcarbodiimide hydrochloride (EDC or EDAC), triethylamine (TEA), p-toluenesulfonic acid (pTSA), 1-hydroxybenzotriazole (HOBt), p-methoxybenzyl (PMB), 4-dimethylaminopyridine (DMAP), Mass Directed Auto-Purification System (MDAP).

### Example 1

### 3-{2-[2-(Benzyloxy)-phenyl]-5-methyl-pyrrol-1-yl}-benzoic acid

### a) 1-(2-Benzyloxy-phenyl)-pentane-1,4-dione

A mixture of 2-benxyloxy-benzaldehyde (3 ml, 1 8.93 mmol), methyl vinyl ketone (1.6 ml, 19.24 mmol), 3-ethyl-5-(2-hydroxyethyl)-4-methylthiazolium bromide (720 mg, 2.86 mmol, 0.1 5 eq) and triethylamine (4 ml, 28.75 mmol) was heated in ethanol (6.3 ml, 3M) at reflux for 24 hours. Upon cooling, the mixture was diluted with EtOAc and washed with saturated NH₄Cl and saturated NaHCO₃, dried (Na₂SO₄) filtered and concentrated. The residue was purified by chromatography with hexane containing a gradient of EtOAc (5-20%) to give the title compound as an oil (3.369g, 63%).

¹H NMR (400 MHz, CDCl₃) 2.16 (3H, s), 2.77 (2H, t, J=6 Hz), 3.27 (2H, t, J=6 Hz), 4.11 (2H, q, J=7 Hz), 5.16 (2H, s), 6.97-7.05 (2H, m), 7.20-7.50 (6H, m's excess), 7.74 (1H, dd, J=2 Hz, J=8 Hz).

### b) 3-[2-(2-Benzyloxy-phenyl)-5methyl-pyrrol-1-yl]-benzoic acid ethyl ester

1-(2-Benzyloxy-phenyl)-pentane-1,4-dione (575 mg, 2.04 mmol), ethyl-3-aminobenzoate (0.37 ml, 2.48 mmol) and pTSA (20 mg) were heated in toluene (20 ml, 0.1M) at reflux for 23 hours. Upon cooling, the mixture was diluted with EtOAc and washed with 2 MHCl and saturated NaHCO₃, dried (Na₂SO₄), filtered and concentrated. The residue was purified by chromatography, with hexane containing a gradient of EtOAc (5-1 0%) as eluant, to give the desired compound (739 mg, 88%).

¹H NMR (400 MHz, CDCl₃) 1.30 (3H, t, J=7 Hz), 2.16 (3H, s), 4.27 (2H, q, J=7 Hz), 4.78 (2H, s), 6.14 (1H, d, J=3 Hz), 6.32 (1H, d, J=3 Hz), 6.65 (1H, d, J=9 Hz), 6.85 (1H, t, J=8 Hz), 7.05-7.15 (4H, m), 7.18-7.32 (5H, m′ excess), 7.76 (1H, s), 7.89 (1H, d, J=8 Hz).

LC/MS t=3.85 min [MH+] 412

### c) 3-[2-(2-Benzyloxy-phenyl)-5-methyl-pyrrol-1-yl]-benzoic acid

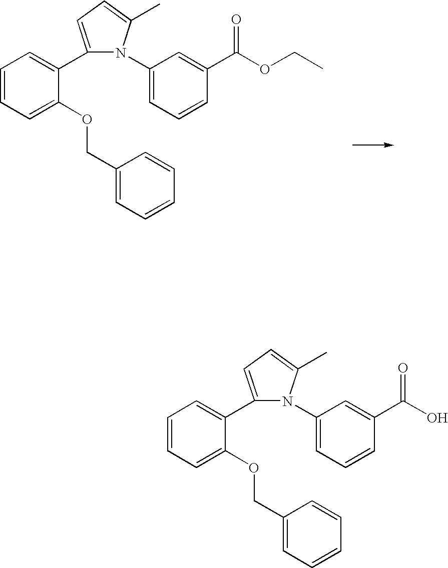

3-[2-(2-Benzyloxy-phenyl)-5-methyl-pyrrol-1-yl]-benzoic acid ethyl ester (203 mg, 0.49 mmol) was heated with DMF (3 ml) and 2M NaOH (2 ml) in a reacti-vial at 85° C. for 24 hrs. The mixture was cooled to room temperature and diluted with EtOAc, washed with 2M HCl then dried (Na₂SO₄), filtered and evaporated to give the title compound (42.7 mg, 23%).

¹H NMR (400 MHz, d6-DMSO) 2.07 (3H, s), 4.84 (2H, s), 6.05 (1H, d, J=3 Hz), 6.18 (1H, d, J=3 Hz), 6.78-6.87 (2H, m), 7.05-7.20 (4H, m), 7.22-7.36 (4H, m), 7.42 (1H, t, J=8 Hz), 7.55 (1H, s), 7.84 (1H, d, J=8 Hz), 13.05 (1H, s).

LC/MS t=3.54 min [MH+] 384, [MH−] 382.

### Example 2

### 3-{2-[5-Chloro-2-(benzyloxy)-phenyl]-5-methyl-pyrrol-1-yl}-benzoic acid

### a) 2-Benzyloxy-5-chloro-benzaldehyde

5-Chlorosalicylaldehyde (10.094g, 64.64 mmol), benzyl bromide (11.5 ml, 96.70 mmol) and K₂CO₃ (17.935g, 13.00 mmol) were heated in DMF (65 ml) at 60° C. for 18 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (15.850g, 100%).

¹H NMR (400 MHz, CDCl₃) 5.18 (2H, s), 7.00 (1H, d, J=9 Hz), 7.32-7.44 (5H, m's excess), 7.47 (1H, dd, J=3 Hz, J=9 Hz), 7.80 (1H, d, J=3 Hz), 10.50 (1H, s).

### b) 1-[5-Chloro-2-(benzyloxy)-phenyl]-pentane-1,4-dione

A mixture of 2-benzyloxy-5-chloro-benzaldehyde (4.044g, 16.41 mmol), methyl vinyl ketone (1.64 ml, 19.73 mmol), 3-ethyl-5-(2-hydroxyethyl)-4-methylthiazolium bromide (654 mg, 2.60 mmol, 0.1 5 eq) and triethylamine (3.42 ml, 28.75 mmol) was heated in ethanol (5.5 ml, 3M) at reflux for 5 hours. Upon cooling, the mixture was diluted with EtOAc and washed with saturated NH₄Cl and saturated NaHCO₃, dried (Na₂SO₄) filtered and concentrated. The residue was purified by chromatography with iso-hexane containing a gradient of EtOAc (5-15%) to give the title compound as an oil (4.011 g, 81%).

¹H NMR (400 MHz, CDCl₃) 2.18 (3H, s), 2.78 (2H, d, J=6 Hz), 3.23 (2H, d, J=6 Hz), 5.15 (2H, s), 6.95 (1H, d, J=9 Hz), 7.23-7.50 (6H, m's excess), 7.70 (1H, d, J=3 Hz).

### c) 3-{2-[5-Chloro-2-(benzyloxy)-phenyl]-5-methyl-pyrrol-1-yl}-benzoic acid ethyl ester

1-[5-Chloro-2-(benzyloxy)-phenyl]-pentane-1,4-dione (1.015g, 3.38 mmol), ethyl-3-aminobenzoate (0.60 ml, 4.02 mmol) and pTSA (50 mg) were heated in toluene (34 ml, 0.1M) at reflux for 2.5 hours. Upon cooling, the mixture was diluted with EtOAc and washed with 2M HCl and saturated NaHCO₃, dried (Na₂SO₄), filtered and concentrated. The residue was purified by chromatography with hexane containing a gradient of EtOAc (5-10%) as eluant, to give the desired compound (906 mg, 60%).

¹H NMR (400 MHz, CDCl₃) 1.31 (3H, t, J=7 Hz), 2.15 (3H, s), 4.28 (2H, q, J=7 Hz), 4.72 (2H, s), 6.13 (1H, d, J=3 Hz), 6.31 (1H, d, J=3 Hz), 6.54 (1H, d, J=9 Hz), 7.00-7.08 (3H, m), 7.12 (1H, d, J=8 Hz), 7.23 (1H, d, J=3 Hz), 7.27-7.34 (4H, m's excess), 7.74 (1H, s), 7.91 (1H, d, J=8 Hz).

### d) 3-{2-[5-Chloro-2-(benzyloxy)-phenyl]-5-methyl-pyrrol-1-yl}-benzoic acid

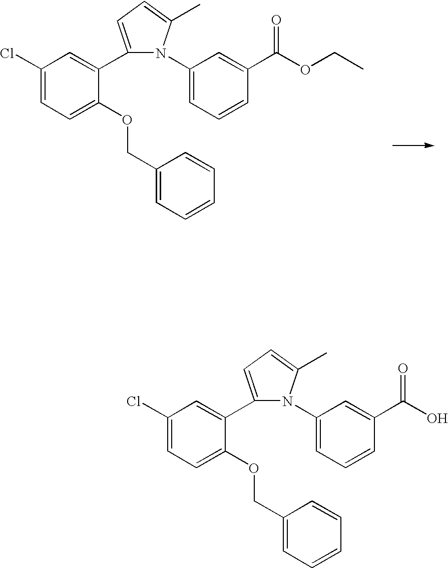

3-{2-[5-Chloro-2-(benzyloxy)-phenyl]-5-methyl-pyrrol-1-yl}-benzoic acid ethyl ester (144.7 mg, 0.32 mmol) was heated in a mixture of EtOH (3 ml) and 2M NaOH (1 ml) at reflux for 15 hours. Upon cooling, the mixture was diluted with EtOAc, washed with 2M HCl, dried (Na₂SO₄), filtered and evaporated to give the title compound (135.1 mg, 100%).

¹H NMR (400 MHz, CDCl₃) 2.16 (3H, s), 4.72 (2H, s), 6.14 (1H, d, J=3 Hz), 6.32 (1H, d, J=3 Hz), 6.56 (1H, d, J=9 Hz), 7.00-7.19 (3H, m), 7.17 (1H, d, J=8 Hz), 7.21-7.35 (5H, m's excess), 7.78 (1H, s), 7.96 (1H, d, J=8 Hz).

### Example 3

### 3-{2-[5-Bromo-2-(benzyloxy)-phenyl]-5-methyl-pyrrol-1-yl}-benzoic acid

### a) 2-Benzyloxy-5-bromo-benzaldehyde

5-Bromosalicylaldehyde (10.045g, 49.98 mmol), benzyl bromide (8.9 ml, 75.00 mmol) and K₂CO₃ (13.800g, 99.99 mmol) were heated in DMF (50 ml, 1M) at 60° C. for 4 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (14.500g, 100%).

¹H NMR (400 MHz, CDCl₃) 5.18 (2H, s), 6.95 (1H, d, J=9 Hz), 7.27-7.50 (5H, m's, excess), 7.60 (1H, dd, J=3 Hz, J=9 Hz), 7.94 (1H, d, J=3 Hz), 10.48 (1H, s).

### b) 1-[5-Bromo-2-(benzyloxy)-phenyl]-pentane-1,4-dione

A mixture of 2-benzyloxy-5-bromo-benzaldehyde (4.079g, 14.02 mmol), methyl vinyl ketone (1.40 ml, 16.84 mmol), 3-ethyl-5-(2-hydroxyethyl)-4-methylthiazolium bromide (589 mg, 2.34 mmol, 0.1 5 eq) and triethylamine (2.93 ml, 21.06 mmol) was heated in ethanol (4.7 ml, 3M) at reflux for 5 hours. Upon cooling, the mixture was diluted with EtOAc and washed with saturated NH₄Cl and saturated NaHCO₃, dried (Na₂SO₄) filtered and concentrated. The residue was purified by chromatography with iso-hexane containing a gradient of EtOAc (5-10%) to give the title compound as an oil (3.780g, 78%).

¹H NMR (400 MHz, CDCl₃) 2.17 (3H, s), 2.77 (2H, t, J=6 Hz), 3.22 (2H, d, J=6 Hz), 5.15 (2H, s), 6.90 (1H, d, J=9 Hz), 7.22-7.48 (5H, m's excess), 7.51 (1H, d, J=1.5 Hz, J=9 Hz), 7.84 (1H, d, J=1.5 Hz).

### c) 3-[2-(5-Bromo-2-benzyloxy-phenyl)-5methyl-pyrrol-1-yl]-benzoic acid ethyl ester

1-[5-Bromo-2-(benzyloxy)-phenyl]-pentane-1,4-dione (1.040g, 3.01 mmol), ethyl-3-aminobenzoate (0.54 ml, 3.62 mmol) and pTSA (50 mg) were heated in toluene (30 ml, 0.1M) at reflux for 4 hours. Upon cooling, the mixture was diluted with EtOAc and washed with 2M HCl and saturated NaHCO₃, dried (Na₂SO₄), filtered and concentrated. The residue was purified by chromatography with hexane containing EtOAc (5%) as eluant, to give the desired compound (811 mg, 55%).

¹H NMR (400 MHz, CDCl₃) 1.31 (3H, t, J=7 Hz), 2.15 (3H, s), 4.28 (2H, q, J=7 Hz), 4.71 (2H, s), 6.13 (1H, d, J=3.5 Hz), 6.31 (1H, d, J=3.5 Hz), 6.49 (1H, d, J=9 Hz), 6.99-7.16 (2H, m), 7.10-7.15 (1H, m), 7.17 (1H, d, J=3 Hz, J=9 Hz), 7.22-7.32 (4H, m's excess), 7.38 (1H, d, J=3 Hz), 7.73 (1H, s), 7.91 (1H, d, J=8 Hz).

### d) 3-{2-[5-Bromo-2-(benzyloxy)-phenyl]-5-methyl-pyrrol-1-yl}-benzoic acid

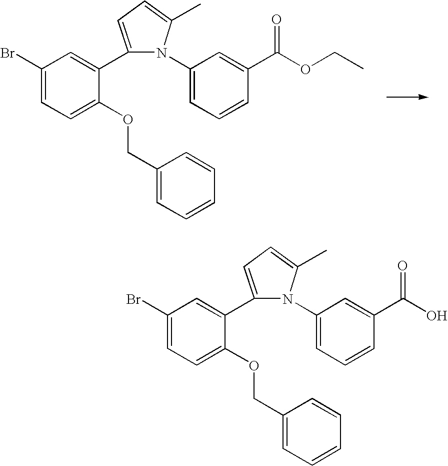

The ethyl ester derivative (144.6 mg, 0.30 mmol) was heated in a mixture of EtOH (3 ml) and 2M NaOH (1.5 ml) at reflux in a reacti-vial for 3 hours. Upon cooling, the mixture was diluted with EtOAc, washed with 2M HCl, dried (Na₂SO₄), filtered and evaporated to give the title compound (136.7 mg, 100%).

¹H NMR (400 MHz, CDCl₃) 2.16 (3H, s), 4.72 (2H, s), 6.14 (1H, d, J=3 Hz), 6.32 (1H, d, J=3 Hz), 6.51 (1H, d, J=9 Hz), 7.00-7.08 (2H, m), 7.13-7.21 (2H, m), 7.22-7.36 (4H, m's excess), 7.39 (1H, d, J=2 Hz), 7.79 (1H, s), 7.96 (1H, d, J=8 Hz).

LC/MS t=3.99 min [MH+] 462 and 463, [MH−] 460 and 461.

### Example 4

### 3-{2-[5-Phenyl-2-(benzyloxy)-phenyl]-5-methyl-pyrrol-1-yl}-benzoic acid

### a) 3-{2-[5-Phenyl-2-(benzyloxy)-phenyl]-5-methyl-pyrrol-1-yl}-benzoic acid ethyl ester

3-{2-[5-Bromo-2-(benzyloxy)-phenyl]-5-methyl-pyrrol-1-yl}-benzoic acid ethyl ester (109.9 mg, 0.21 mmol), benzene boronic acid (52 mg, 0.43 mmol), K₂CO₃ (230 mg, 1.67 mmol) and tetrakistriphenylphophine palladium (0) (26.0 mg, 0.02 mmol) were heated in a toulene (1 ml) and EtOH (1 ml) at 90° C. in a reacti-vial for 6 hours. Upon cooling, the mixture was diluted with EtOAc and washed with H₂O, dried (Na₂SO₄), filtered and concentrated. the residue was purified by chromatography, with iso-hexane containing a gradient of EtOAc (1.5-5%) to give the title compound (63.4 mg, 58%).

¹H NMR (400 MHz, CDCl₃) 1.27 (3H, q, J=7 Hz), 2.18 (3H, s), 4.27 (2H, q, J=7 Hz), 4.84 (2H, s), 6.16 (1H, d, J=3 Hz), 6.40 (1H, d, J=3 Hz), 6.72 (1H, d, J=9 Hz), 7.10-7.15 (2H, m), 7.17-7.42 (11H, m's excess), 7.47 (1H, d, J=2 Hz), 7.82 (1H, s), 7.92 (1H, d, J=8 Hz).

LC/MS t=4.31 min [MH+] 488.

### b) 3-{2-[5-Phenyl-2-(benzyloxy)-phenyl]-5-methyl-pyrrol-1-yl}-benzoic acid

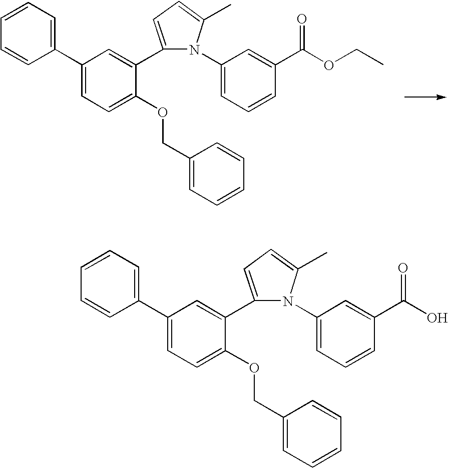

3-{2-[5-Phenyl-2-(benzyloxy)-phenyl]-5-methyl-pyrrol-1-yl}-benzoic acid ethyl ester (62.1 mg, 0.13 mmol) was heated in a mixture of EtOH (1.2 ml) and 2M NaOH (0.6 ml) at 90° C. in a reacti-vial for 2 hours. Upon cooling, the mixture was diluted with EtOAc, washed with 2M HCl, dried (Na₂SO₄), filtered and evaporated to give the title compound (58.3 mg, 100%).

¹H NMR (400 MHz, CDCl₃) 2.19 (3H, s), 4.83 (2H, s), 6.17 (1H, d, J=3 Hz), 6.40 (1H, d, J=3 Hz), 6.74 (1H, d, J=8 Hz), 7.12 (1H, d, J=8 Hz), 7.20-7.45 (12H, m's excess), 7.48 (1H, d, J=2 Hz), 7.86 (1H, t, J=3 Hz), 7.96 (1H, d, J=8 Hz).

LC/MS t=4.08 min [MH+] 460, [MH−] 458.

### Example 5

### 3-{2-[6-Chloro-2-(cyclohexylmethoxy)-phenyl]-5-methyl-pyrrol-1-yl}-benzoic acid

### a) 5-Chloro-2-cyclohexylmethoxy-benzaldehyde

5-Chlorosalicylaldehyde (5.025g, 32.08 mmol), cyclohexylmethyl bromide (4.70 ml, 33.70 mmol) and K₂CO₃ (8.890g, 64.42 mmol) were heated in DMF (32 ml) at 60° C. for 18 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (6.115g, 85%).

¹H NMR (400 MHz, CDCl₃) 1.12-1.39 (5H, m), 1.66-1.92 (6H, m), 3.85 (2H, d, J=6 Hz), 6.92 (1H, d, J=9 Hz), 7.46 (1H, dd, J=3 Hz, J=9 Hz), 7.78 (1H, d, J=3 Hz), 10.47 (1H, s).

### b) 1-[5-Chloro-2-(cyclohexylmethoxy)-phenyl]-pentane-1,4-dione

A mixture of 2-benzyloxy-5-bromo-benzaldehyde (2.003g, 7.93 mmol), methyl vinyl ketone (0.80 ml, 9.62 mmol), 3-ethyl-5-(2-hydroxyethyl)-4-methylthiazolium bromide (307 mg, 1.22 mmol, 0.15 eq) and triethylamine (1.65 ml, 11.86 mmol) was heated in ethanol (2.4 ml, 3M) at reflux for 5 hours. Upon cooling, the mixture was diluted with EtOAc and washed with saturated NH₄Cl and saturated NaHCO₃, dried (Na₂SO₄) filtered and concentrated. The residue was purified by chromatography with iso-hexane containing a gradient of EtOAc (5-10%) to give the title compound as an oil (1.2153g, 48%).

¹H NMR (400 MHz, CDCl₃) F2944 1.02-1.39 (5H, m), 1.61-1.95 (6H, m), 2.26 (3H, s), 2.85 (2H, t, J=6 Hz), 3.27 (2H, t, J=6 Hz), 3.84 (2H, d, J=6 Hz), 6.88 (1H, d, J=9 Hz), 7.37 (1H, dd, J=3 Hz, J=9 Hz), 7.70 (1H, d, J=3 Hz).

### c) 3-{2-[5-Chloro-2-(cyclohexylmethoxy)-phenyl]-5-methyl-pyrrol-1-yl}-benzoic acid ethyl ester

1-[5-Chloro-2-(cyclohexylmethoxy)-phenyl]-pentane-1,4-dione (478.9 mg, 1.49 mmol), ethyl-3-aminobenzoate (0.27 ml, 1.81 mmol) and pTSA (25 mg) were heated in toluene (1 5 ml, 0.1M) at reflux for 3 hours. Upon cooling, the mixture was diluted with EtOAc and washed with 2M HCl and saturated NaHCO₃, dried (Na₂SO₄), filtered and concentrated. The residue was purified by chromatography, with hexane containing a gradient of EtOAc (1-2%) as eluant, to give the desired compound (547 mg, 81%).

¹H NMR (400 MHz, CDCl₃) 0.76-0.93 (2H, m), 1.06-1.30 (3H, m's excess), 1.37 (3H, t, J=7 Hz), 1.52-1.75 (6H, m's excess), 2.19 (3H, s), 3.40 (2H, d, J=6 Hz), 4.35 (2H, q, J=7 Hz), 6.11 (1H, d, J=3 Hz), 6.28 (1H, d, J=3 Hz), 6.59 (1H, d, J=9 Hz), 7.05 (1H, dd, J=3 Hz, J=9 Hz), 7.09 (1H, d, J=3 Hz), 7.17 (1H, d, J=8 Hz), 7.31 (1H, t, J=8 Hz), 7.83 (1H, s), 7.91 (1H, d, J=8 Hz).

### d) 3-{2-[5-Chloro-2-(cyclohexylmethoxy)-phenyl]-5-methyl-pyrrol-1-yl}-benzoic acid

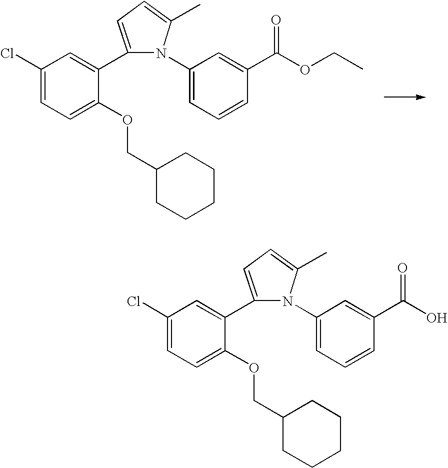

3-{2-[5-Chloro-2-(cyclohexylmethoxy)-phenyl]-5-methyl-pyrrol-1-yl}-benzoic acid ethyl ester (149.0 mg, 0.33 mmol) was heated in a mixture of EtOH (3 ml) and 2M NaOH (1.5 ml) at 90° C. in a reacti-vial for 2 hours. Upon cooling, the mixture was diluted with EtOAc, washed with 2M HCl, dried (Na₂SO₄), filtered and evaporated to give the title compound.

¹H NMR (400 MHz, CDCl₃) 0.78-0.93 (2H, m), 1.06-1.31 (3H, m's excess), 1.54-1.74 (6H, m's excess), 2.19 (3H, s), 3.42 (2H, d, J=6 Hz), 6.12 (1H, d, J=3 Hz), 6.29 (1H, d, J=3 Hz), 6.60 (1H, d, J=8 Hz), 7.06 (1H, dd, J=3 Hz, J=8 Hz), 7.10 1H, d, J=3 Hz), 7.22 (1H, d, J=8 Hz), 7.35 (1H, t, J=8 Hz), 7.92 (1H, s), 7.97 (1H, d, J=8 Hz).

LC/MS t=4.08, [MH+] 424 and 426, [MH−] 422 and 424.

### Example 6

### 4-{2-[5-Chloro-2-(benzyloxy)-phenyl]-5-methyl-pyrrol-1-yl}-methanesulfonyl benzene

1-[5-Chloro-2-(benzyloxy)-phenyl]-pentane-1,4-dione (251 mg, 0.83 mmol), 4-methylsulfonylaniline hydrochloride (209 mg, 1.01 mmol) and triethylamine (0.11 ml, 0.79 mmol) were heated in toluene (8.3 ml, 0.1M) at reflux for 4 hours. Upon cooling, the mixture was diluted with EtOAc and washed with 2 M HCl and saturated NaHCO₃, dried (Na₂SO₄), filtered and concentrated. The residue was purified by chromatography, with hexane containing a gradient of EtOAc (10-25%) as eluant, to give the desired compound (1 88 mg, 50%).

¹H NMR (400 MHz, CDCl₃) 2.16 (3H, s), 3.05 (3H, s), 4.66 (2H, s), 6.15 (1H, s, J=3 Hz), 6.34 (1H, d, J=3 Hz), 6.60 (1H, d, J=9 Hz), 7.01-7.13 (5H, m), 7.23 (1H, d, J=2 Hz), 7.28-7.36 (3H, m), 7.74 (2H, d, J=9 Hz).

LC/MS t=3.88 min, [MH+] 452 and 454.

### Example 7

### 3-{2-[5-Chloro-2-(benzyloxy)-phenyl]-4-methyl-pyrrol-1-yl}-methanesulfonyl benzene

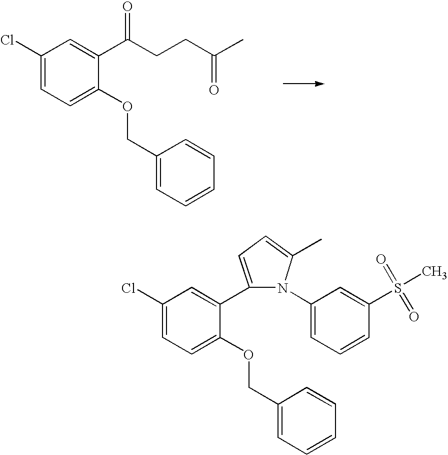

1-[5-chloro-2-(benzyloxy)-phenyl]-pentane-1,4-dione (279 mg, 0.93 mmol), 3-methylsulfonylaniline hydrochloride (262 mg, 1.26 mmol) and triethylamine (0.12 ml, 0.86 mmol) were heated in toluene (9.3 ml, 0.1M) at reflux for 4.5 hours. Upon cooling, the mixture was diluted with EtOAc and washed with 2M HCl and saturated NaHCO₃, dried (Na₂SO₄), filtered and concentrated. The residue was purified by chromatography, with hexane containing a gradient of EtOAc (10-30%) as eluant, to give the desired compound (270 mg, 64%).

¹H NMR (400 MHz, CDCl₃) 2.19 (2H, m), 2.66 (3H, s), 4.75 (2H, s), 6.15 (1H, d, J=3 Hz), 6.34 (1H, d, J=3 Hz), 6.57 (1H, d, J=9 Hz), 6.95-7.10 (3H, m), 7.17-7.34 (5H, m's excess), 7.42 (1H, t, J=8 Hz), 7.55 (1H, s), 7.79 (1H, d, J=8 Hz).

LC/MS t=3.86 min [MH+] 452 and 454.

### Example 8

### 3-{2-[5-Bromo-2-(4-methoxy-benzyloxy)-phenyl]-5-methyl-pyrrol-1-yl}-benzoic acid

### a) 5-Bromo-2-(4-methoxy-benzyloxy)-benzaldehyde

5-Bromo-2-hydroxybenzaldehyde (8.56 g, 0.043 mol, 1 eq) was added to DMF (60 ml).

K₂CO₃ (11.75 g, 0.085 mol, 2 eq) and 4-methoxybenzyl chloride (10 g, 0.06 mol, 1.5 eq) were added to the stirred reaction mixture. After complete addition the vessel was heated to 60° C. After 3 hours the reaction had gone to completion. The reaction mixture was quenched with water (250 ml) and washed with EtOAc (2×250 ml). The organic extracts were combined and washed with brine (150 ml), dried over MgSO₄ and the solvent was then removed in vacuo to yield title compound (13.9 g, 0.04 mol, 100%) as a white solid.

¹H NMR (400 MHz, CDCl₃) 3.82 (3H, s), 5.10 (2H, s), 6.93 (2H, d, J=8.2 Hz), 6.96 (1H, d, J=9.0 Hz), 7.34 (2H, d, J=8.2 Hz), 7.60 (1H, dd, J=2.2, 8.8 Hz), 7.92 (1H, d, J=2.2 Hz), 10.5 (1H, s).

LC/MS t=3.62 min [M+NH₄⁺] 337.9.

### b) 1-[5-Bromo-2-(4-methoxy-benzyloxy)-phenyl]-pentane-1,4-dione

5-Bromo-2-(4-methoxy-benzyloxy)-benzaldehyde (9.45 g, 0.029 mol, 1 eq) was added to EtOH (9 ml). TEA (12.25 ml, 0.088 mol, 3 eq), methyl vinyl ketone (2.10 g, 0.03 mol, 1.02 eq) and 3ethyl-5-(2-hydroxyethyl)-4-methylthiazolium bromide (2.22 g, 8.80 mmol, 0.3 eq) were added to the stirred reaction mixture. After complete addition the vessel was heated to reflux. After 18 hours the reaction had gone to completion. The reaction mixture was quenched with saturated NH₄Cl solution (300 ml) and washed with EtOAc (2×250 ml). The organic extracts were combined and washed with saturated NaHCO₃ solution (250 ml) and brine (200 ml), dried over MgSO₄ and the solvent was then removed in vacuo to yield a dark oil. The crude product was purified by chromatography on silica gel (20% EtOAc/iso-hexane) to yield title compound (6.42 g, 0.016 mol, 57%) as a yellow oil which crystallised to form a yellow solid upon cooling.

¹H NMR (400 MHz, CDCl₃) 2.17 (3H, s), 2.76 (2H, t, J=6.0 Hz), 3.19 (2H, t, J=6.0 Hz), 3.82 (3H, s), 5.08 (2H, s), 6.91 (3H, m), 7.34 (2H, d, J=8.2 Hz), 7.51 (1H, dd, J=2.2, 8.4 Hz), 7.82 (1H, d, J=2.2 Hz).

LC/MS t=3.55 mins [PMB⁺] 121.

### c) 3-{2-[5-Bromo-2-(4-methoxy-benzyloxy)-phenyl]-5-methyl-pyrrol-1-yl}-benzoic acid ethyl ester

1-[5-Bromo-2-(4-methoxy-benzyloxy)-phenyl]-pentane-1,4dione (8.017 g, 0.02 mol, 1 eq) and ethyl-3-aminobenzoate (3.67 ml, 0.025 mol, 1.2 eq) were combined in toluene (3 ml). After complete addition the vessel was heated to reflux. After 12 hours the reaction had gone to completion. The remaining solvent was removed in vacuo to yield a dark oil. The crude product was purified by chromatography on silica gel (11% EtOAc:iso-hexane) to yield title compound (6.252 g, 0.012 mol, 60%) as a yellow oil.

¹H NMR (400 MHz, CDCl₃) 1.31 (3H t, J=7.0 Hz), 2.14 (3H, s), 3.79 (3H, s), 4.29 (2H, q, J=7.0 Hz), 4.63 (2H, s), 6.12 (1H, d, J=3.0 Hz), 6.29 (1H, d, J=3.3 Hz), 6.51 (1H, d, J=8.4 Hz), 6.81 (2H, d, J=8.6 Hz), 6.98 (2H, d, J=8.2 Hz), 7.11 (1H, br d, J=8.0 Hz), 7.17 (1H, dd, J=2.2, 8.4 Hz), 7.28 (1H, t, J=7.6 Hz), 7.37 (1H, d, J=2.2 Hz), 7.72 (1H, t, J=1.5 Hz), 7.91 (1H, br d, J=7.8 Hz).

LC/MS t=4.22 mins [MH⁺] 519.9.

### d) 3-{2-[5-Bromo-2-(4-methoxy-benzyloxy)-phenyl]-5-methyl-pyrrol-1-yl}-benzoic acid

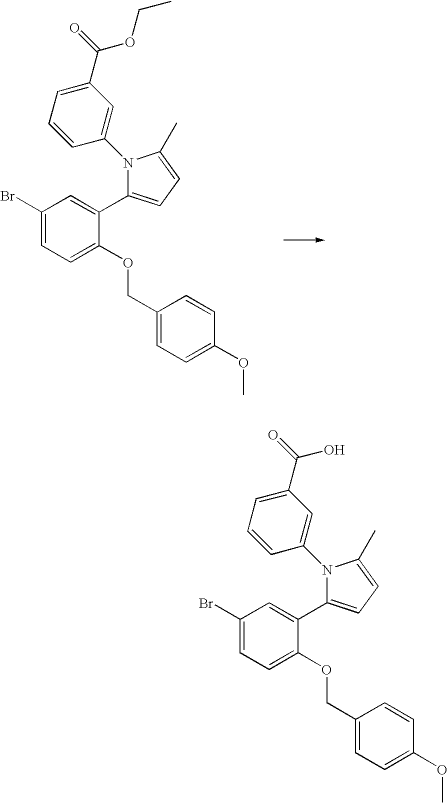

3-{2-[5-Bromo-2-(4-methoxy-benzyloxy)-phenyl]-5-methyl-pyrrol-1-yl}-benzoic acid ethyl ester (150 mg, 0.29 mmol, 1 eq) was added to EtOH (3 ml) and 2M NaOH (1.5 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (131 mg, 0.27 mmol, 92%) as a yellow oil.

¹H NMR (400 MHz, CDCl₃) 2.14 (3H, s), 3.78 (3H, s), 4.64 (2H, s), 6.12 (1H, d, J=3.0 Hz), 6.30 (1H, d, J=3.0 Hz), 6.54 (1H, d, J=8.2 Hz), 6.81 (2H, d, J=8.2 Hz), 6.99 (2H, d, J=8.2 Hz), 7.15 (1H, br d, J=8.0 Hz), 7.18 (1H, dd, J=2.0, 8.6 Hz), 7.30 (1H, t, J=7.8 Hz), 7.36 (1H, d, J=2.0 Hz), 7.77 (1H, br s), 7.95 (1H, br d, J=7.9 Hz).

LC/MS t=3.95 mins [MH⁺] 492.

### Example 9

### 3-{2-[5-Bromo-2-(3,4-dichloro-benzyloxy)-phenyl]-5-methyl-pyrrol-1-yl}-benzoic acid

### a) 3-{2-[5-Bromo-2-(hydroxy)-phenyl]-methyl-pyrrol-1-yl}-benzoic acid ethyl ester

A solution of 3-{2-[5-Bromo-2-(4-methoxy-benzyloxy)-phenyl]-5-methyl-pyrrol-1-yl}-benzoic acid ethyl ester. (5.95 g, 0.011 mol, 1 eq) in 4.0 M HCl in dioxane (30 ml) was stirred for 30 minutes at room temperature under N₂. After this time the reaction had gone to completion and the solvent was removed in vacuo to yield the crude product as a dark oil. The crude product was purified by chromatography on silica gel (10% EtOAc:iso-hexane) to yield title compound (817 mg, 2.04 mmol, 18%) as an orange oil.

¹H NMR (400 MHz, CDCl₃) 1.38 (3H, t, J=7.0 Hz), 2.16 (3H, s), 4.36 (2H, q, J=7.0 Hz), 5.97 (1H, s), 6.15 (1H, d, J=3.6 Hz), 6.35 (1H, d, J=3.6 Hz), 6.73 (1H, d, J=8.4 Hz), 6.89 (1H, d, J=2.2 Hz), 7.15 (1H, dd, J=2.0, 8.2 Hz), 7.22 (1H, br d), 7.39 (1H, t, J=7.8 Hz), 7.81 (1H, t, J=1.5 Hz), 7.99 (1H, br d, J=7.8 Hz).

LC/MS t=3.79 mins [MH⁺] 400.

### b) 3-{2-[5-Bromo-2-(3,4-dichloro-benzyloxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (130 mg, 0.33 mmol, 1 eq) was added to DMF (1.3 ml). K₂CO₃ (92 mg, 0.66 mmol, 2 eq) and 3,4-dichlorobenzyl bromide (85.7 μL, 0.5 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (3% EtOAc:iso-hexane) to yield title compound (104 mg, 0.19 mmol, 57%) as a colourless oil.

¹H NMR (400 MHz, CDCl₃) 1.31 (3H, t, J=7.1 Hz), 2.18 (3H, s), 4.29 (2H, q, J=7.0 Hz), 4.63 (2H, s), 6.15 (1H, d, J=3.2 Hz), 6.30 (1H, d, J=3.3 Hz), 6.46 (1H, d, J=8.8 Hz), 6.87 (1H, dd, J=1.8, 8.0 Hz), 7.12 (1H, br s), 7.13 (1H, br d, J=7.0 Hz), 7.20 (1H, dd, J=2.0, 8.2 Hz), 7.30 (1H, t, J=8.0 Hz), 7.35 (1H, d, J=8.0 Hz), 7.42 (1H, d, J=2.0 Hz), 7.72 (1H, t, J=1.2 Hz), 7.91 (1H, br d, J=7.8 Hz).

LC/MS t=4.48 mins [MH⁺] 558

### c) 3-{2-[5-Bromo-2-(3,4-dichloro-benzyloxy)-phenyl]-5-methyl-pyrrol-1-yl}-benzoic acid

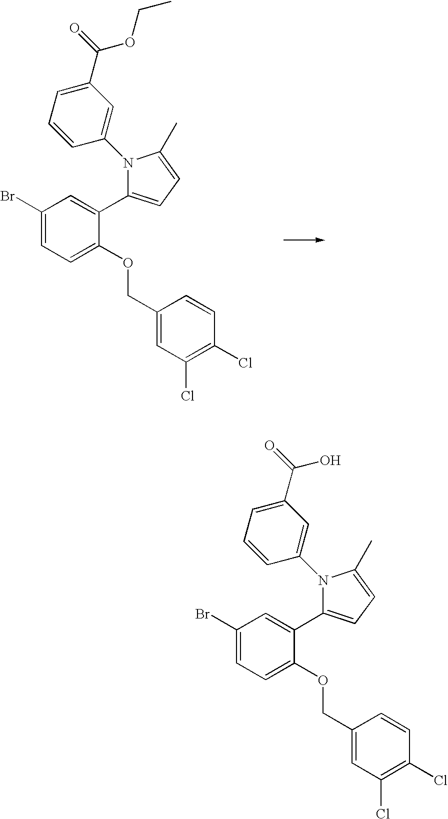

3-{2-[5-Bromo-2-(3,4-dichloro-benzyloxy)-phenyl]-5-methyl-pyrrol-1-yl}-benzoic acid ethyl ester (100 mg, 0.18 mmol, 1 eq) was added to EtOH (2 ml) and 2M NaOH (1 ml) in a reactivial. The vessel was heated to reflux. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (86 mg, 0.27 mmol, 91%) as a yellow oil.

¹H NMR (400 MHz, CDCl₃) 2.18 (3H, s), 4.65 (2H, s), 6.16 (1H, d, J=3.2 Hz), 6.31 (1H, d, J=3.4 Hz), 6.48 (1H, d, J=8.8 Hz), 6.89 (1H, dd, J=1.8, 8.1 Hz), 7.14 (1H, d, J=1.8 Hz), 7.17 (1H, br d, J=8.0 Hz), 7.21 (1H, dd, J=2.0, 8.4 Hz), 7.34 (1H, t, J=7.8 Hz), 7.35 (1H, d, J=8.0 Hz), 7.42 (1H, d, J=2.0 Hz), 7.77 (1H, br s), 7.98 (1H, br d, J=7.8 Hz).

LC/MS t=4.28 mins [MH⁺] 530.

### Example 10

### 3-{2-[5-Bromo-2-(2-chloro-4-fluoro-benzyloxy)-phenyl]-5-methyl-pyrrol-1}-yl)--benzoic acid

### a) 3-{2-[5-Bromo-2-(2-chloro-4-fluoro-benzyloxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (130 mg, 0.33 mmol, 1 eq) was added to DMF (1.3 ml). K₂CO₃ (92 mg, 0.66 mmol, 2 eq) and 2-chloro-fluorobenzyl bromide (111.74 mg, 0.5 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product purified by chromatography on silica gel (3% EtOAc:iso-hexane) to yield title compound (122 mg, 67%) as a colourless oil.

¹H NMR (400 MHz, CDCl₃) 1.31 (3H, t, J=7.0 Hz), 2.17 (3H, s), 4.29 (2H, q, J=7.0 Hz), 4.73 (2H, s), 6.14 (1H, d, J=3.2 Hz), 6.31 (1H, d, J=3.6 Hz), 6.47 (1H, d, J=8.4 Hz), 6.89 (2H, m), 7.09 (1H, dd, J=1.6, 8.0 Hz), 7.14 (1H, br d, J=7.6 Hz), 7.20 (1H, dd, J=2.4, 8.5 Hz), 7.31 (1H, t, J=7.8 Hz), 7.40 (1H, d, J=2.0 Hz), 7.73 (1H, t, J=1.4 Hz), 7.92 (1H, br d, J=7.8 Hz).

LC/MS t=4.39 mins [MH⁺] 541.9.

### b) 3-{2-[5-Bromo-2-(2-chloro-4-fluoro-benzyloxy)-phenyl]-5-methyl-pyrrol-1-yl}-benzoic acid

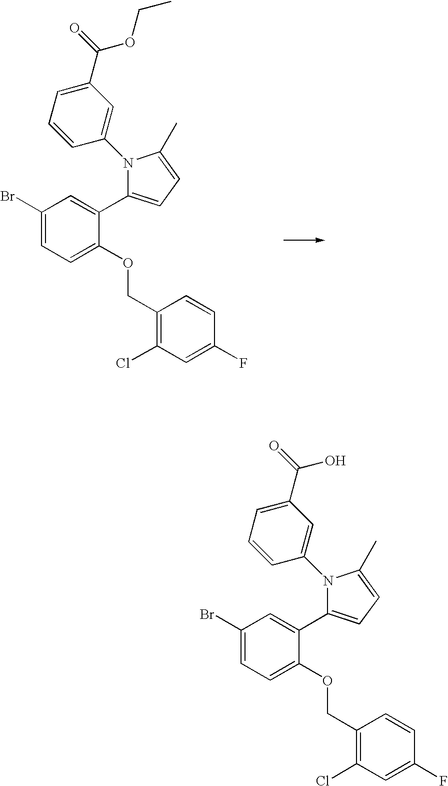

3-{2-[5-Bromo-2-(2-chloro-4-fluoro-benzyloxy)-phenyl]-5-methyl-pyrrol-1-yl}-benzoic acid ethyl ester (100 mg, 0.18 mmol, 1 eq) was added to EtOH (2 ml) and 2M NaOH (1 ml) in a reactivial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (95 mg, 100%) as a yellow oil.

¹H NMR (400 MHz, CDCl₃) 2.17 (3H, s), 4.74 (2H, s), 6.15 (1H, d, J=3.0 Hz), 6.31 (1H, d, J=3.0 Hz), 6.50 (1H, d, J=8.4 Hz), 6.91 (2H, m), 7.07 (1H, dd, J=2.0, 8.0 Hz), 7.21 (2H, m), 7.34 (1H, t, J=7.8 Hz), 7.39 (1H, d, J=2.0 Hz), 7.79 (1H, t, J=1.8 Hz), 7.98 (1H, br d, J=7.8 Hz).

LC/MS t=4.16 mins [MH⁺] 513.8.

### Example 11

### 3-{2-[5-Bromo-2-(4-fluoro-benzyloxy)-phenyl]-5-methyl-pyrrol-1-yl}-benzoic acid

### a) 3-{2-[5-Bromo-2-(4-fluoro-benzyloxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (130 mg, 0.33 mmol, 1 eq) was added to DMF (1.3 ml). K₂CO₃ (92 mg, 0.66 mmol, 2 eq) and 4-fluorobenzyl bromide (61.8 μL, 0.5 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (7% EtOAc:iso-hexane) to yield title compound (114 mg, 69%) as a colourless oil.

¹H NMR (400 MHz, CDCl₃) 1.31 (3H, t, J=7.0 Hz), 2.16 (3H, s), 4.29 (2H, q, J=7.0 Hz), 4.72 (2H, s), 6.13 (1H, d, J=3.2 Hz), 6.29 (1H, d, J=3.2 Hz), 6.48 (1H, d, J=8.4 Hz), 6.98 (4H, m), 7.10 (1H, br d, J=7.8 Hz), 7.19 (1H, dd, J=2.0, 8.7 Hz), 7.28 (1H, t, J=7.8 Hz), 7.39 (1H, d, J=2.4 Hz), 7.70 (1H, br s), 7.90 (1H, br d, J=7.6 Hz).

LC/MS t=4.25 mins [MH⁺] 508.0.

### b) 3-{2-[5-Bromo-2-(4-fluoro-benzyloxy)-phenyl]-5-methyl-pyrrol-1-yl}-benzoic acid

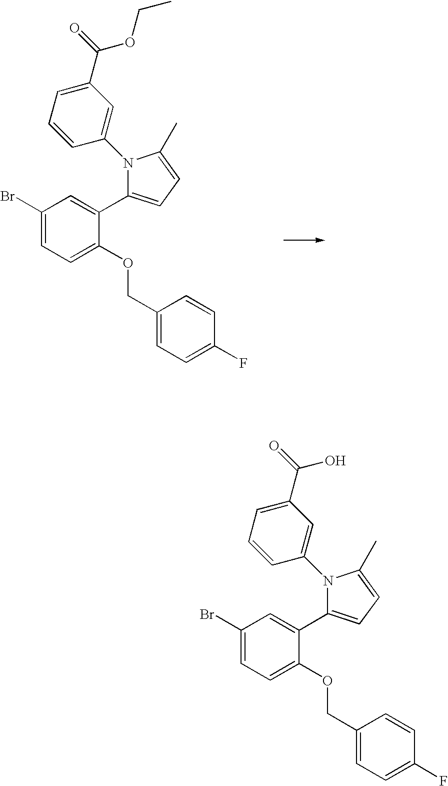

3-{2-[5-Bromo-2-(4-fluoro-benzyloxy)-phenyl]-5-methyl-pyrrol-1-yl}-benzoic acid ethyl ester (100 mg, 0.20 mmol, 1 eq) was added to EtOH (2 ml) and 2M NaOH (1 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (91 mg, 96%) as a yellow oil.

¹H NMR (400 MHz, CDCl₃) 2.16 (3H, s), 4.68 (2H, s), 6.13 (1H, dd, J=0.2, 3.0 Hz), 6.30 (1H, d, J=3.0 Hz), 6.52 (1H, d, J=8.4 Hz), 6.96 (2H, br t, J=8.2 Hz), 7.02 (2H, dd, J=7.0, 8.2 Hz), 7.15 (1H, br d, J=8.0 Hz), 7.20 (1H, dd, J=2.2, 8.4 Hz), 7.31 (1H, t, J=7.8 Hz), 7.38 (1H, d, J=2.2 Hz), 7.76 (1H, t, J=1.6 Hz), 7.97 (1H, br d, J=7.6 Hz).

LC/MS t=3.98 mins [MH⁺] 479.9

### Example 12

### 3-{2-[5-Bromo-2-(2,4-difluoro-benzyloxy)-phenyl]-5-methyl-pyrrol-1-yl}-benzoic acid

### a) 3-{2-[5-Bromo-2-(2,4-difluoro-benzyloxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (130 mg, 0.33 mmol, 1 eq) was added to DMF (1.3 ml). K₂CO₃ (92 mg, 0.66 mmol, 2 eq) and 2,4-difluorobenzyl bromide (69.0 μL, 0.5 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (7% EtOAc:iso-hexane) to yield title compound (116 mg, 67%) as a colourless oil.

¹H NMR (400 MHz, CDCl₃) 1.32 (3H, t, J=7.0 Hz), 2.15 (3H, s), 4.30 (2H, q, J=7.0 Hz), 4.72 (2H, s), 6.12 (1H, d, J=3.2 Hz), 6.29 (1H, d, J=3.4 Hz), 6.53 (1H, d, J=8.4 Hz), 6.78 (2H, m), 6.95 (1H, dt, J=6.4, 9.0 Hz), 7.12 (1H, br d, J=8.0 Hz), 7.21 (1H, dd, J=2.2, 8.8 Hz), 7.29 (1H, t, J=7.7 Hz), 7.37 (1H, d, J=2.4 Hz), 7.72 (1H, br s), 7.90 (1H, br d, J=7.9 Hz).

LC/MS t=4.27 mins [MH⁺] 525.9.

### b) 3-{2-[5-Bromo-2-(2,4difluoro-benzyloxy)-phenyl]-5-methyl-pyrrol-1-yl}-benzoic acid

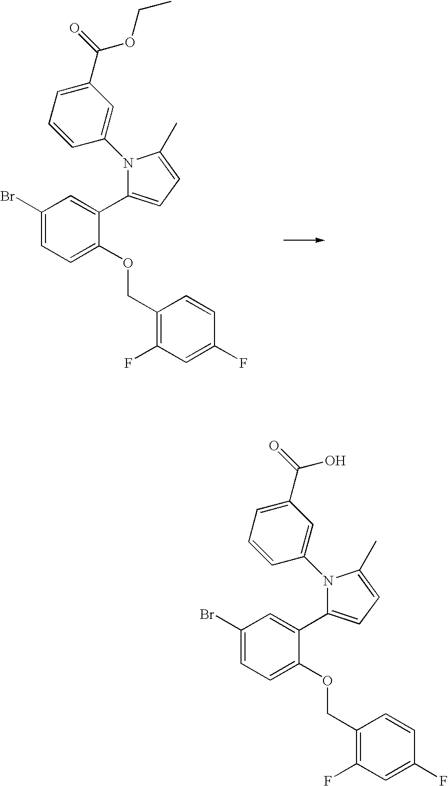

3-{2-[5-Bromo-2-(2,4-difluoro-benzyloxy)-phenyl]-5-methyl-pyrrol-1-yl}-benzoic acid ethyl ester (100 mg, 0.20 mmol, 1 eq) was added to EtOH (2 ml) and 2M NaOH (1 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (85 mg, 90%) as a yellow oil.

¹H NMR (400 MHz, CDCl₃) 2.16 (3H, s), 4.73 (2H, s), 6.13 (1H, d, J=3.0 Hz), 6.30 (1H, d, J=3.0 Hz), 6.56 (1H, d, J=8.4 Hz), 6.79 (2H, m), 6.99 (1H, q, J=7.8 Hz), 7.17 (1H, br d, J=8.0 Hz), 7.22 (1H, dd, J=2.2, 8.6 Hz), 7.34 (1H, t, J=8.0 Hz), 7.37 (1H, d, J=2.2 Hz), 7.78 (1H, br s), 7.98 (1H, br d, J=7.7 Hz).

LC/MS t=4.01 mins [MH⁺] 497.9.

### Example 13

### 3-{2-[5-Bromo-2-(4-chloro-benzyloxy)-phenyl]-5-methyl-pyrrol-1-yl}-benzoic acid

### a) 3-{2-[5-Bromo-2-(4-chloro-benzyloxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (130 mg, 0.33 mmol, 1 eq) was added to DMF (1.3 ml). K₂CO₃ (92 mg, 0.66 mmol, 2 eq) and 4-chlorobenzyl bromide (102.7 mg, 0.5 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (7% EtOAc:iso-hexane) to yield title compound (126 mg, 74%) as a colourless oil.

¹H NMR (400 MHz, CDCl₃) 1.32 (3H, t, J=7.0 Hz), 2.16 (3H, s), 4.29 (2H, q, J=7.0 Hz), 4.66 (2H, s), 6.13 (1H, d, J=3.2 Hz), 6.29 (1H, d, J=3.4 Hz), 6.47 (1H, d, J=8.2 Hz), 6.95 (2H, d, J=8.0 Hz), 7.12 (1H, br d, J=7.8 Hz), 7.18 (1H, dd, J=2.2, 8.4 Hz), 7.24 (2H, d, J=8.0 Hz), 7.28 (1H, t, J=7.8 Hz), 7.40 (1H, d, J=2.2 Hz), 7.71 (1H, br s), 7.91 (1H, br d, J=7.8 Hz).

LC/MS t=4.38 mins [MH⁺] 523.9.

### b) 3-{2-[5-Bromo-2-(4-chloro-benzyloxy)-phenyl]-5-methyl-pyrrol-1-yl}-benzoic acid

3-{2-[5-Bromo-2-(4-chloro-benzyloxy)-phenyl]-5-methyl-pyrrol-1-yl}-benzoic acid ethyl ester (100 mg, 0.20 mmol, 1 eq) was added to EtOH (2 ml) and 2M NaOH (1 ml) in a reactivial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (93 mg, 98%) as a yellow oil.

¹H NMR (400 MHz, CDCl₃) 2.16 (3H, s), 4.68 (2H, s), 6.13 (1H, d, J=3.0 Hz), 6.30 (1H, d, J=3.2 Hz), 6.49 (1H, d, J=8.4 Hz), 6.98 (2H, d, J=8.2 Hz), 7.16 (1H, br d, J=7.8 Hz), 7.19 (1H, dd, J=2.2, 8.4 Hz), 7.25 (2H, d, J=8.0 Hz), 7.32 (1H, t, J=7.8 Hz), 7.39 (1H, d, J=2.2 Hz), 7.77 (1H, br s), 7.97 (1H, br d, J=7.8 Hz).

LC/MS t=4.14 mins [MH⁺] 495.8.

### Example 14

### 3-{2-[2-(4-Methoxy-benzyloxy)-phenyl]-pyrrol-1-yl}-benzoic acid

### a) 2-(4-Methoxy-benzyloxy)-benzaldehyde

2-Hydroxybenzaldehyde (5.20 g, 0.043 mol, 1 eq) was added to DMF (60 ml). K₂CO₃ (11.75 g, 0.085 mol, 2 eq) and 4-methoxybenzyl chloride (10 g, 0.06 mol, 1.5 eq) were added to the stirred reaction mixture. After complete addition the vessel was heated to 60° C. After 3 hours the reaction had gone to completion. The reaction mixture was quenched with water (250 ml) and washed with EtOAc (2×250 ml). The organic extracts were combined and washed with brine (150 ml), dried over MgSO₄ and the solvent was then removed in vacuo to yield title compound (13.9 g, 0.04 mol, 100%) as a white solid.

¹H NMR (400 MHz, CDCl₃) 3.82 (3H, s), 5.12 (2H, s), 6.93 (2H, d, J=8.2 Hz), 7.05 (2H, m), 7.36 (2H, d, J=8.2 Hz), 7.52 (1H, dt, J=1.8, 8.0 Hz), 7.85 (1H, dd, J=1.8, 8.0 Hz), 10.5 (1H, s).

LC/MS t=3.31 mins [PMB⁺] 120.9.

### b) 1-[2-(4-Methoxy-benzyloxy)-phenyl]-pentane-1,4-dione

2-(4-Methoxy-benzyloxy)-benzaldehyde (2.50 g, 7.23 mmol, 1 eq) was added to EtOH (2.2 ml). Et₃N (1.51 ml, 10.8 mmol, 1.5 eq), methyl vinyl ketone (516.5 mg, 7.38 mmol, 1.02 eq) and 3-ethyl-5-(2-hydroxyethyl)-4-methylthiazolium bromide (2.22 g, 8.80 mmol, 0.3 eq) were added to the stirred reaction mixture. After complete addition the vessel was heated to reflux. After 24 hours the reaction had gone to completion. The reaction mixture was quenched with saturated NH₄Cl solution (200 ml) and washed with EtOAc (2×200 ml). The organic extracts were combined and washed with saturated NaHCO₃ solution (150 ml) and brine (150 ml), dried over MgSO₄ and the solvent was then removed in vacuo to yield a dark oil. The crude product was purified by chromatography on silica gel (20% EtOAc/iso-hexane) to yield title compound (1.27 g, 56%) as a yellow oil.

¹H NMR (400 MHz, CDCl₃) 2.15 (3H, s), 2.73 (2H, t, J=6.1 Hz), 3.23 (2H, t, J=6.1 Hz), 3.81 (3H, s), 5.08 (2H, s), 6.91 (2H, d, J=7.0 Hz), 7.00 (2H, m), 7.37 (2H, d, J=7.0 Hz), 7.42 (1H, dt, J=1.8, 8.0 Hz), 7.72 (1H, d, J=1.8, 7.9 Hz).

LC/MS t=3.28 mins [PMB⁺] 120.9.

### c) 3-{2-[2-(4-Methoxy-benzyloxy)-phenyl]-5-methyl-pyrrol-1-yl}-benzoic acid ethyl ester

1-[2-(4-Methoxy-benzyloxy)-phenyl]-pentane-1,4-dione (6.76 g, 0.022 mol, 1 eq) and ethyl-3-aminobenzoate (3.88 ml, 0.026 mol, 1.2 eq) were combined in toluene (2.2 ml). After complete addition the vessel was heated to reflux. After 12 hours the reaction had gone to completion. The remaining solvent was removed in vacuo to yield a dark oil. The crude product was purified by chromatography on silica gel (11% EtOAc:iso-hexane) to yield title compound (5.79 g, 61%) as a yellow solid.

¹H NMR (400 MHz, CDCl₃) 1.30 (3H, t, J=7.0 Hz), 2.16 (3H, s), 3.79 (3H, s), 4.29 (2H, q, J=7.0 Hz), 4.71 (2H, s), 6.13 (1H, d, J=3.4 Hz), 6.30 (1H, d, J=3.5 Hz), 6.68 (1H, d, J=8.0 Hz), 6.81 (2H, d, J=8.4 Hz), 6.85 (1H, dt, J=0.9, 7.2 Hz), 7.03 (2H, d, J=8.2 Hz), 7.09 (2H, m), 7.19 (1H, dd, J=1.8, 7.8 Hz), 7.23 (1H, t, J=7.8 Hz), 7.76 (1H, t, J=1.4 Hz), 7.88 (1H, br d, J=7.8 Hz).

LC/MS t=4.04 mins [M+NH₄+] 407.9.

### d) 3-{2-[2-(4-Methoxy-benzyloxy)-phenyl]-pyrrol-1-yl}-benzoic acid

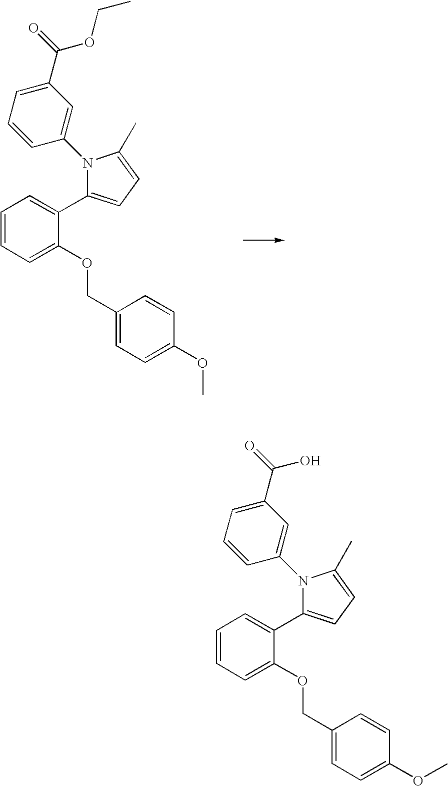

3-{2-[2-(4-Methoxy-benzyloxy)-phenyl]-5-methyl-pyrrol-1-yl}-benzoic acid ethyl ester (150 mg, 0.34 mmol, 1 eq) was added to EtOH (3 ml) and 2M NaOH (1.5 ml) in a reactivial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (125 mg, 0.27 mmol, 89%) as a yellow oil.

¹H NMR (400 MHz, CDCl₃) 2.16(3H, s), 3.77 (3H, s), 4.70 (2H, s), 6.12 (1H, d, J=3.3 Hz), 6.30 (1H, d, J=3.3 Hz), 6.69 (1H, d, J=8.0 Hz), 6.82 (2H, d, J=8.2 Hz),6.86 (1H, t, J=7.6 Hz), 7.03 (2H, d, J=8.2 Hz), 7.10 (1H, dt, J=1.6, 8.0 Hz), 7.15 (1H, br d, J=8.0 Hz), 7.22 (1H, dd, J=1.6, 7.9 Hz), 7.27 (1H, t, J=7.8 Hz), 7.77 (1H, br s), 7.92 (1H, br d, J=7.8 Hz).

LC/MS t=3.73 min [MH⁺] 414.0.

### Example 15

### 3-{2-[2-(3,4-Dichloro-benzyloxy)-phenyl]-5-methyl-pyrrol-1-yl}-benzoic acid

### a) 3-{2-[2-(Hydroxy)-phenyl]-5-methyl-pyrrol-1-yl}-benzoic acid ethyl ester

A solution of 3-{2-[2-(4-Methoxy-benzyloxy)-phenyl]-5-methyl-pyrrol-1-yl}-benzoic acid ethyl ester. (3.96 g, 0.009 mol, 1 eq) in 4.0 M HCl in dioxane (20 ml) was stirred for 30 minutes at room temperature under N₂. After this time the reaction had gone to completion and the solvent was removed in vacuo to yield the crude product as a dark oil. The crude product was purified by chromatography on silica gel (10% EtOAc:iso-hexane) to yield title compound (1.242 g, 45%) as a brown solid.

¹H NMR (400 MHz, CDCl₃) 1.38 (3H, t, J=7.0 Hz), 2.16 (3H, s), 4.35 (2H, q, J=7.0 Hz), 5.98 (1H, s), 6.17 (1H, d, J=3.8 Hz), 6.37 (1H, d, J=3.8 Hz), 6.61 (1H, dt, J=1.0, 7.8 Hz), 6.71 (1H, dd, J=1.6, 7.8 Hz), 6.88 (1H, dd, J=1.0, 8.0 Hz), 7.16 (1H, dt, J=1.8, 8.0 Hz), 7.18 (1H, br d), 7.35 (1H, t, J=7.8 Hz), 7.83 (1H, t, J=1.5 Hz), 7.94 (1H, br d, J=7.8 Hz).

LC/MS t=3.54 mins [MH⁺] 322.1.

### b) 3-{2-[2-(3,4-Dichloro-benzyloxy)-phenyl]-5-methyl-pyrrol-1-yl}-benzoic acid ethyl ester

3-{2-[2-(Hydroxy)-phenyl]-5-methyl-pyrrol-1-yl}-benzoic acid ethyl ester (200 mg, 0.62 mmol, 1 eq) was added to DMF (2 ml). K₂CO₃ (172 mg, 1.25 mmol, 2 eq) and 3,4 dichlorobenzyl bromide (160.2 1 μL, 0.93 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (5% EtOAc:iso-hexane) to yield title compound (225 mg, 75%) as a colourless oil.

¹H NMR (400 MHz, CDCl₃) 1.30 (3H, t, J=7.1 Hz), 2.18 (3H, s), 4.28 (2H, q, J=7.0 Hz), 4.70 (2H, s), 6.16 (1H, d, J=3.2 Hz), 6.30 (1H, d, J=3.0 Hz), 6.60 (1H, d, J=8.2 Hz), 6.90 (2H, m), 7.11 (2H, m), 7.18 (1H, d, J=1.6 Hz), 7.26 (2H, m), 7.35 (1H, d, J=8.0 Hz), 7.74 (1H, br s), 7.88 (1H, br d, J=7.8 Hz).

LC/MS t=4.32 mins [MH⁺] 480.0.

### c) 3-{2-[2-(3,4-Dichloro-benzyloxy)-phenyl]-5-methyl-pyrrol-1-yl}-benzoic acid

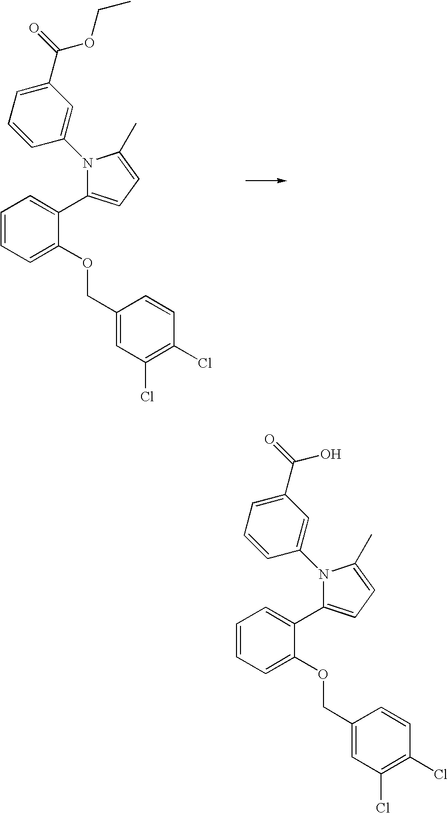

3-{2-[2-(3,4-Dichloro-benzyloxy)-phenyl]-5-methyl-pyrrol-1-yl}-benzoic acid ethyl ester (150 mg, 0.31 mmol, 1 eq) was added to EtOH (3 ml) and 2M NaOH (1.5 ml) in a reactivial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (140 mg, 99%) as a yellow oil.

¹H NMR (400 MHz, CDCl₃) 2.19 (3H, s), 4.70 (2H, s), 6.17 (1H, d, J=3.0 Hz), 6.30 (1H, d, J=3.0 Hz), 6.61 (1H, d, J=8.0 Hz), 6.91 (2H, m), 7.13 (2H, m), 7.18 (1H, d, J=1.4 Hz), 7.28 (2H, m), 7.35 (1H, d, J=8.0 Hz), 7.78 (1H, br s), 7.94 (1H, br d, J=7.8 Hz).

LC/MS t=4.32 mins [MH⁺] 451.9.

### Example 16

### 3-{2-[2-(2-Chloro-4-fluoro-benzyloxy)-phenyl]-5-methyl-pyrrol-1-yl}-benzoic acid

### a) 3-{2-[2-(2-Chloro-4-fluoro-benzyloxy)-phenyl]-5-methyl-pyrrol-1-yl}-benzoic acid ethyl ester

3-{2-[2-(Hydroxy)-phenyl]-5-methyl-pyrrol-1-yl}-benzoic acid ethyl ester (200 mg, 0.62 mmol, 1 eq) was added to DMF (2 ml). K₂CO₃ (172 mg, 1.25 mmol, 2 eq) and 2-chloro-4-fluorobenzyl bromide (208.9 mg, 0.93 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (5% EtOAc:iso-hexane) to yield title compound (189 mg, 65%) as a colourless oil.

¹H NMR (400 MHz, CDCl₃) 1.30 (3H, t, J=7.1 Hz), 2.18 (3H, s), 4.28 (2H, q, J=7.0 Hz), 4.79 (2H, s), 6.15 (1H, d, J=3.4 Hz), 6.31 (1H, d, J=3.4 Hz), 6.61 (1H, d, J=8.0 Hz), 6.88 (2H, m), 6.98 (1H, dd, J=7.0, 8.2 Hz), 7.08 (1H, dd, J=2.0, 8.0 Hz), 7.12 (2H, m), 7.26 (2H, m), 7.77 (1H, br s), 7.89 (1H, br d, J=7.6 Hz).

LC/MS t=4.23 mins [MH⁺] 464.0.

### b) 3-{2-[2-(2-Chloro-4-fluoro-benzyloxy)-phenyl]-5-methyl-pyrrol-1-yl}-benzoic acid

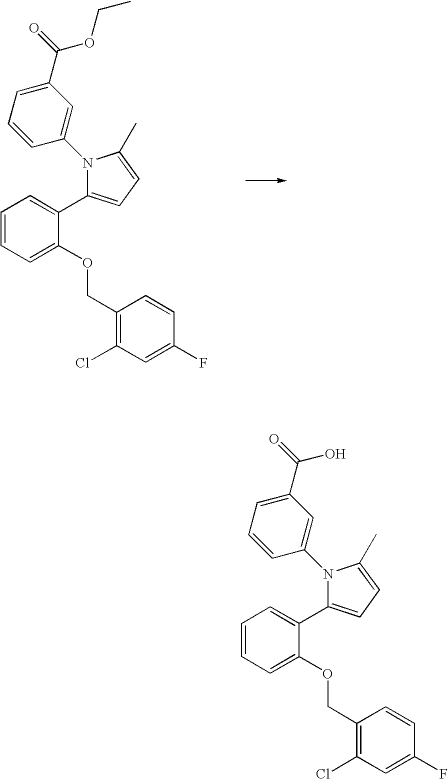

3-{2-[2-(2-Chloro-4-fluoro-benzyloxy)-phenyl]-5-methyl-pyrrol-1-yl}-benzoic acid ethyl ester (150 mg, 0.32 mmol, 1 eq) was added to EtOH (3 ml) and 2M NaOH (1.5 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (126 mg, 89%) as a yellow oil.

¹H NMR (400 MHz, CDCl₃) 2.18 (3H, s), 4.79 (2H, s), 6.16 (1H, d, J=3.4 Hz), 6.32 (1H, d, J=3.4 Hz), 6.64 (1H, d, J=8.2 Hz), 6.90 (2H, m), 7.00 (1H, dd, J=7.0, 8.2 Hz), 7.06 (1H, dd, J=2.2, 8.0 Hz), 7.13 (1H, dt, J=1.6, 7.8 Hz), 7.20 (1H, br d, J=8.0 Hz), 7.25 (1H, dd, J=1.4, 8.0 Hz), 7.30 (1H, t, J=7.8 Hz), 7.79 (1H, br s), 7.95 (1H, br d, J=7.8 Hz).

LC/MS t=3.96 mins [MH⁺] 436.0.

### Example 17

### 3-{2-[2-(4-Fluoro-benzyloxy)-phenyl]-5-methyl-pyrrol-1-yl}-benzoic acid

### a) 3-{2-[2-(4-Fluoro-benzyloxy)-phenyl]-5-methyl-pyrrol-1-yl}-benzoic acid ethyl ester

3-{2-[2-(Hydroxy)-phenyl]-5-methyl-pyrrol-1-yl}-benzoic acid ethyl ester (200 mg, 0.62 mmol, 1 eq) was added to DMF (2 ml). K₂CO₃ (172 mg, 1.25 mmol, 2 eq) and 4-fluorobenzyl bromide (115.4 μL, 0.93 mmol, 1.6 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (5% EtOAc:iso-hexane) to yield title compound (175 mg, 65%) as a colourless oil.

¹H NMR (400 MHz, CDCl₃) 1.30 (3H, t, J=7.0 Hz), 2.16 (3H, s), 4.28 (2H, q, J=7.0 Hz), 4.72 (2H, s), 6.13 (1H, d, J=3.0 Hz), 6.30 (1H, d, J=3.0 Hz), 6.64 (1H, d, J=8.2 Hz), 6.86 (1H, dt, J=0.4, 7.4 Hz), 6.97 (2H, t, J=8.2 Hz), 7.08 (4H, m), 7.23 (2H, m), 7.72 (1H, t, J=1.8 Hz), 7.88 (1H, br d, J=7.8 Hz).

LC/MS t=4.07 mins [MH⁺] 430.1.

### b) 3-{2-[2-(4-Fluoro-benzyloxy)-phenyl]-5-methyl-pyrrol-1-yl}-benzoic acid

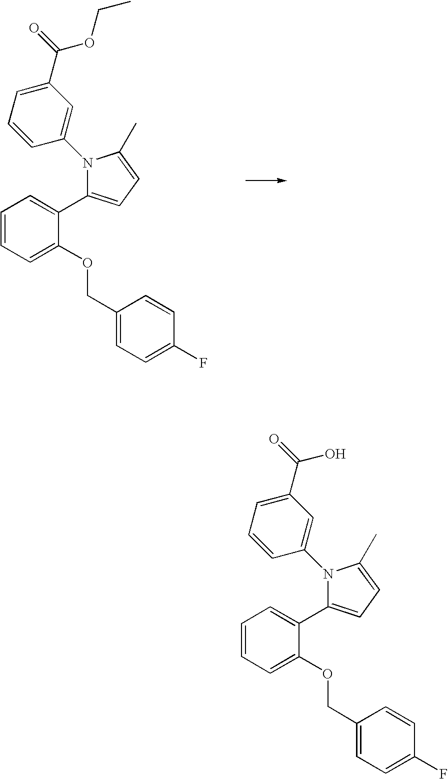

3-{2-[2-(4-Fluorobenzyloxy)-phenyl]-5-methyl-pyrrol-1-yl}-benzoic acid ethyl ester (150 mg, 0.35 mmol, 1 eq) was added to EtOH (3 ml) and 2M NaOH (1.5 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (121 mg, 86%) as a yellow oil.

¹H NMR (400 MHz, CDCl₃) 2.17 (3H, s), 4.72 (2H, s), 6.15 (1H, d, J=3.2 Hz), 6.30 (1H, d, J=3.0 Hz), 6.66 (1H, d, J=8.0 Hz), 6.88 (1H, br d, J=7.6 Hz), 6.97 (2H, dd, J=8.0, 9.6 Hz), 7.10 (4H, m), 7.26 (2H, m), 7.77 (1H, t, J=1.6 Hz), 7.93 (1H, br d, J=7.8 Hz).

LC/MS t=3.77 mins [MH⁺] 402.1.

### Example 18

### 3-{2-[2-(2,4-Difluoro-benzyloxy)-phenyl]-5-methyl-pyrrol-1-yl}-benzoic acid

### a) 3-{2-[2-(2,4-Difluoro-benzyloxy)-phenyl]-5-methyl-pyrrol-1-yl}-benzoic acid ethyl ester

3-{2-[2-(Hydroxy)-phenyl]-5-methyl-pyrrol-1-yl}-benzoic acid ethyl ester (200 mg, 0.62 mmol, 1 eq) was added to DMF (2 ml). K₂CO₃ (172 mg, 1.25 mmol, 2 eq) and 2,4-difluorobenzyl bromide (129.0 μL, 0.93 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (3% EtOAc:iso-hexane) to yield title compound (183 mg, 66%) as a colourless oil.

¹H NMR (400 MHz, CDCl₃) 1.31 (3H, t, J=7.0 Hz), 2.16 (3H, s), 4.29 (2H, q, J=7.0 Hz), 4.79 (2H, s), 6.13 (1H, d, J=3.0 Hz), 6.30 (1H, d, J=3.2 Hz), 6.68 (1H, d, J=8.0 Hz), 6.78 (2H, m), 6.87 (1H, dt, J=0.4, 7.6 Hz), 7.02 (1H, dt, J=6.2, 9.0 Hz), 7.11 (2H, m), 7.21 (1H, dd, J=1.6, 7.8 Hz), 7.25 (1H, t, J=7.8 Hz), 7.75 (1H, t, J=1.8 Hz), 7.89 (1H, br d, J=7.8 Hz).

LC/MS t=4.10 mins [MH⁺] 448.1

### b) 3-{2-[2-(2,4-Difluoro-benzyloxy)-phenyl]-5-methyl-pyrrol-1-yl}-benzoic acid

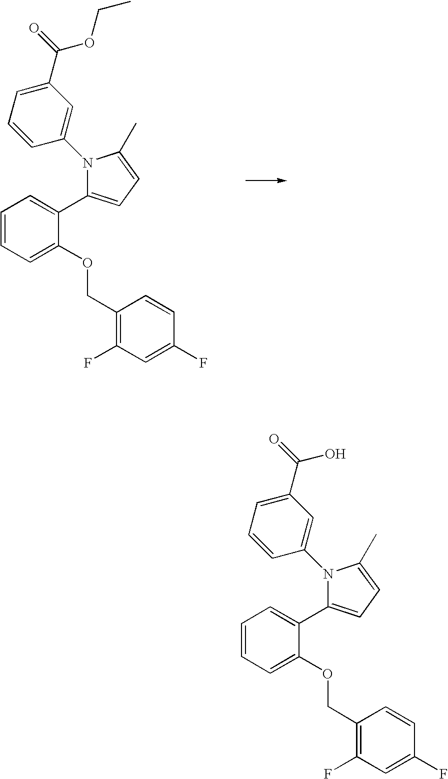

3-{2-[2-(2,4-Difluoro-benzyloxy)-phenyl]-5-methyl-pyrrol-1-yl}-benzoic acid ethyl ester (150 mg, 0.34 mmol, 1 eq) was added to EtOH (3 ml) and 2M NaOH (1.5 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (131 mg, 93%) as a yellow oil.

¹H NMR (400 MHz, d6-DMSO) 2.03 (3H, s), 4.87 (2H, s), 6.02 (1H, d, J=3.0 Hz), 6.17 (1H, d, J=3.2 Hz), 6.80 (1H, br t, J=7.5 Hz), 6.91 (1H, d, J=8.0 Hz), 7.01 (1H, br d, J=7.0 Hz), 7.07 (1H, dt, J=2.0, 8.0 Hz), 7.11 (2H, m), 7.25 (2H, m), 7.34 (1H, t, J=7.8 Hz), 7.53 (1H, br s), 7.81 (1H, br d, J=7.4 Hz).

LC/MS t=3.80 mins [MH⁺] 420.0.

### Example 19

### 3-{2-[2-(4-Chloro-benzyloxy)-phenyl]-5-methyl-Pyrrol-1-yl}-benzoic acid

### a) 3-{2-[2-(4-Chloro-benzyloxy)-phenyl]-5-methyl-pyrrol-1-yl}-benzoic acid ethyl ester

3-{2-[2-(Hydroxy)-phenyl]-5-methyl-pyrrol-1-yl}-benzoic acid ethyl ester (200 mg, 0.62 mmol, 1 eq) was added to DMF (2 ml). K₂CO₃ (172 mg, 1.25 mmol, 2 eq) and 4-chlorobenzyl bromide (192.0 mg, 0.93 mmol, 1.5 eq) were added to the stirred reaction mixture. After complete addition the vessel was heated to 60° C. After 3 hours the reaction had gone to completion. The reaction mixture was quenched with water (20 ml) and washed with EtOAc (2×20 ml). The organic extracts were combined and washed with brine (10 ml), dried over MgSO₄ and the solvent was then removed in vacuo. The crude product was purified by chromatography on silica gel (3% EtOAc:iso-hexane) to yield title compound (179 mg, 64%) as a colourless oil.

¹H NMR (400 MHz, CDCl₃) 1.30 (3H, t, J=7.0 Hz), 2.17 (3H, s), 4.28 (2H, q, J=7.0 Hz), 4.72 (2H, s), 6.13 (1H, d, J=3.2 Hz), 6.30 (1H, d, J=3.0 Hz), 6.61 (1H, d, J=7.8 Hz), 6.87 (1H, dt, J=0.4, 7.8 Hz), 7.00 (2H, d, J=8.2 Hz), 7.10 (2H, m), 7.24 (4H, m), 7.74 (1H, t, J=1.8 Hz), 7.88 (1H, br d, J=7.8 Hz).

LC/MS t=4.20 mins [MH⁺] 446.0.

### b) 3-{2-[2-(4-Chloro-benzyloxy)-phenyl]-5-methyl-pyrrol-1-yl}-benzoic acid

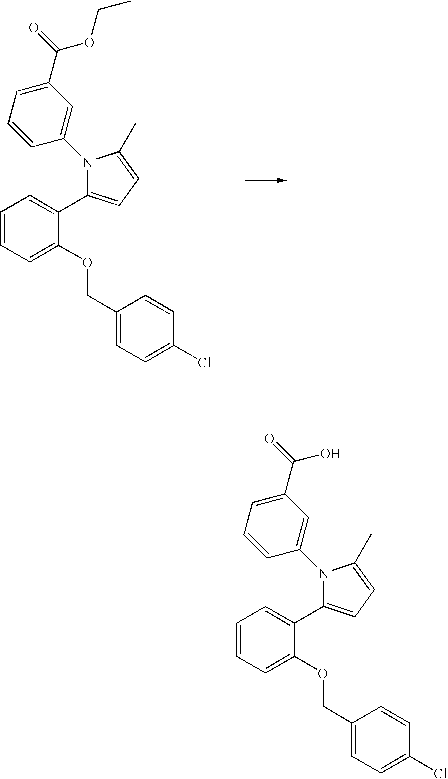

3-{2-[2-(4-Chloro-benzyloxy)-phenyl]-5-methyl-pyrrol-1-yl}-benzoic acid ethyl ester (150 mg, 0.34 mmol, 1 eq) was added to EtOH (3 ml) and 2M NaOH (1.5 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (140 mg, 100%) as a yellow oil.

¹H NMR (400 MHz, CDCl₃) 2.17 (3H, s), 4.72 (2H, s), 6.15 (1H, d, J=3.2 Hz), 6.30 (1H, d, J=3.2 Hz), 6.62 (1H, d, J=8.2 Hz), 6.89 (1H, br t, J=7.6 Hz), 7.02 (2H, d, J=8.0 Hz), 7.11 (1H, dt, J=1.4, 8.0 Hz), 7.11 (1H, br d, J=7.8 Hz), 7.26 (4H, m), 7.78 (1H, br s), 7.93 (1H, br d, J=7.8 Hz).

LC/MS t=3.92 mins [MH⁺] 418.0.

### Example 20

### 3-{2-[5-Chloro-2-(4-methoxy-benzyloxy)-phenyl]-5-methyl-pyrrol-1-yl}-benzoic acid

### a) 5-Chloro-2-(4-methoxy-benzyloxy)-benzaldehyde

5-Chloro-2-hydroxy-benzaldehyde (3.08g, 19.7 mmol), 4methoxybenzyl chloride (4 ml, 29.5 mmol) and potassium carbonate (5.43g, 39.3 mmol) were heated in DMF at 60° C. in a nitrogen atmosphere for 2 hours. Upon cooling the reaction mixture was diluted with EtOAc and washed with water. The organic layer was extracted and the aqueous layer washed with EtOAc (3×100 ml). The combined organic extracts were then washed with brine and dried over MgSO₄, filtered and concentrated in vacuo. This yielded the title compound as a white solid (6.64g, 100%+PMB-OH).

¹H-NMR (400 MHz, CDCl₃) 3.82 (3H, s), 5.11 (2H, s), 6.93 (2H, d, J=9 Hz), 7.02 (1H, d, J=9 Hz), 7.35 (2H, d, J=9 Hz), 7.44 (1H, dd, J=3 Hz, 9 Hz), 7.79 (1H, d, 3 Hz), 10.4 (1H, s)

LC/MS t=3.56 min.

### b) 1-[S-Chloro-2-(4-methoxy-benzyloxy)-phenyl]-pentane-1,4-dione

5-Chloro-2-(4-methoxy-benzyloxy)-benzaldehyde (6.25g, 80%+PMB-OH, 18.1 mmol), triethylamine (7.54 ml, 54.2 mmol), methyl vinyl ketone (1.53 ml, 1 8.4 mmol) and 3-ethyl-5-(2-hydroxyethyl)-4-ethylthiazolium bromide (1.37g, 5.4 mmol) were refluxed in EtOH (7 ml) for 18 hours. Upon cooling the reaction mixture was diluted with EtOAc and washed with saturated NH₄Cl. The organic layer was extracted and the aqueous layer washed with EtOAc (3×200 ml). The combined organic extracts were then washed with saturated NaHCO₃ and brine and then dried over MgSO₄, filtered and concentrated in vacuo. The residue was purified by chromatography on silica gel eluting with 15% EtOAc/iso-hexane. This yielded the title compound as a yellow solid (4.33g, 69%).

¹H-NMR (400 MHz, CDCl₃) 1.58 (3H, s), 2.76 (2H, t, J=6 Hz), 3.20 (2H, t, J=6 Hz), 3.83 (3H, s), 5.08 (2H, s) 6.92 (2H, d, J=9 Hz), 6.98 (1H, d, J=9 Hz), 7.36 (2H, d, J=9 Hz), 7.39 (1H, d, J=3 Hz), 7.69 (1H, d, J=3 Hz).

LC/MS t=3.47 min

### c) 3-{2-[5-Chloro-2-(4-methoxy-benzyloxy)-phenyl]-5-methyl-pyrrol-1-yl}-benzoic acid ethyl ester

1-[5-Chloro-2-(4-methoxy-benzyloxy)-phenyl]-pentane-1,4-dione (3g, 8.7 mmol) and ethyl-3-aminobenzoate (1.55 ml, 10.4 mmol) were heated in toluene (1 ml) in a sealed vessel at 150° C. for 40 hours. Upon cooling the solvent was removed in vacuo. The residue was purified by chromatography on silica gel eluting with 10% EtOAc/iso-hexane. This yielded the title compound as a yellow oil (2.41g, 59%).

¹H-NMR (400 MHz, CDCl₃) 1.32 (3H, t, J=7 Hz), 2.15 (3H, s), 3.80 (3H, s) 4.29 (2H, q, J=7 Hz), 4.66 (2H, s), 6.12 (1H, d, J=3 Hz), 6.30 (1H, d, J=3 Hz), 6.58 (1H, d, J=9 Hz), 6.81(2H, d, J=9 Hz) 6.99 (2H, d, J=9 Hz), 7.03 (1H, dd, J=3 Hz, 9 Hz), 7.09-7.12 (1H, m) 7.21 (1H, d, J=3 Hz), 7.28 (1H, t, J=8 Hz), 7.72 (1H, t, J=1 Hz), 7.91 (1H, dt, J=1 Hz, 8 Hz).

LC/MS t=4.18 [MH⁺] 476 min.

### d) 3-{2-[5-Chloro-2-(4-methoxy-benzyloxy)-phenyl]-5-methyl-pyrrol-1-yl}-benzoic acid

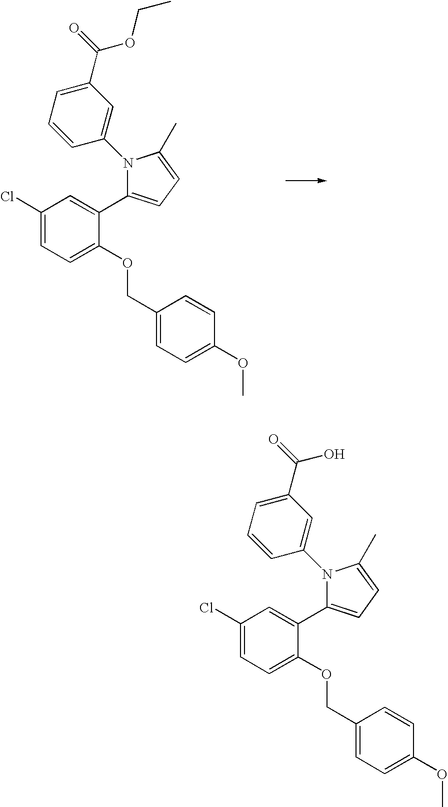

3-{2-[5-Chloro-2-(4-methoxy-benzyloxy)-phenyl]-5-methyl-pyrrol-1-yl}-benzoic acid ethyl ester (150 mg, 0.29 mmol, 1 eq) was added to EtOH (3 ml) and 2M NaOH (1.5 ml) in a reacti-vial. The vessel was heated to 100° C. After 2 hours the reaction had gone to completion. The reaction mixture was quenched with 2M HCl solution (10 ml) and washed with EtOAc (2×10 ml). The organic extracts were combined and washed with brine (10 ml), dried over MgSO₄ and the solvent was then removed in vacuo to yield the title compound (100 mg, 71%) as a yellow oil.

¹H NMR (400 MHz, CDCl₃) 2.15 (3H, s), 3.78 (3H, s), 4.65 (2H, s), 6.12 (1H, d, J=3.0 Hz), 6.30 (1H, d, J=3.1 Hz), 6.59 (1H, d, J=8.4 Hz), 6.81 (2H, d, J=8.2 Hz), 7.00 (2H, d, J=8.2 Hz), 7.05 (1H, dd, J=2.0, 8.2 Hz), 7.15 (1H, br d, J=7.8 Hz), 7.22 (1H, d, J=2.2 Hz), 7.31 (1H, t, J=7.8 Hz), 7.77 (1H, br s), 7.96 (1H, br d, J=8.0 Hz).

LC/MS t=3.91 mins [MH⁺] 448.0.

### Example 21

### 3-{2-[5-Chloro-2-(4-chloro-benzyloxy)-phenyl]-5-methyl-pyrrol-1-yl}-benzoic acid

### a) 3-{2-[5-Chloro-2-(hydroxy)-phenyl]-5-methyl-pyrrol-1-yl}-benzoic acid ethyl ester

3-{2-[5-Chloro-2-(4-methoxy-benzyloxy)-phenyl]-5-methyl-pyrrol-1-yl}-benzoic acid ethyl ester (2.5g, 5.2 mmol) was stirred at room temperature and under nitrogen in 4.0M hydrogen chloride in dioxane (15 ml) for 15 minutes. The solvent was then removed in vacuo and the residue diluted with EtOAc. The solution was then washed with saturated NaHCO₃ and brine, dried over MgSO₄, filtered and concentrated in vacuo. The resultant oil was purified by chromatography on silica gel eluting with 10% EtOAc/iso-hexane. This yielded the title compound as a yellow oil (0.886g, 48%).

¹H-NMR (400 MHz, CDCl₃) 1.38 (3H, t, J=7 Hz), 2.16 (3H, s), 4.36 (2H, q, J=7 Hz), 5.87 (1H, s), 6.17(1H, d, J=3 Hz), 6.36 (1H, d, J=3 Hz), 6.72 (1H, d, J=3 Hz), 6.80 (1H, d, 9 Hz), 7.02 (1H, dd, J=3 Hz, 9 Hz), 7.19-7.22 (1H, m), 7.40 (1H, t, J=8 Hz), 7.81 (1H, t, J=1 Hz), 7.99 (1H, dt, J=1 Hz, 8 Hz).

LC/MS t=3.75 min [MH⁺] 356/358.

### b) 3-{2-[5-Chloro-2-(4-chloro-benzyloxy)-phenyl]-5-methyl-pyrrol-1-yl}-benzoic acid ethyl ester

3-{2-[5-Chloro-2-(hydroxy)-phenyl]-5-methyl-pyrrol-1-yl}-benzoic acid ethyl ester (0.148g, 0.41 mmol), 4-chlorobenzyl bromide (0.128g, 0.62 mmol) and potassium carbonate (0.12g, 0.82 mmol) were heated in DMF at 60° C. in a nitrogen atmosphere for 18 hours. Upon cooling the mixture was diluted with EtOAc and water. The organic layer was extracted and the aqueous layer washed with EtOAc. The combined organics were then washed with brine and concentrated in vacuo. This yielded the title compound as a clear oil (0.1 12g, 56%).The residue was purified by chromatography on silica gel eluting with 5% EtOAc/iso hexane.

¹H NMR (400 MHz, CDCl₃) 1.31 (3H, t, J=7 Hz), 2.15 (3H, s), 4.28 (2H, q, J=7 Hz), 4.67 (2H, s), 6.14 (1H, d, J=3 Hz), 6.30 (1H, d, J=3 Hz), 6.52 (1H, d, J=9 Hz), 6.97 (2H, d, J=9 Hz), 7.05 (1H, dd, J=3 Hz, 9 Hz), 7.09-7.12 (1H, m), 7.22-7.30 (4H, m′ excess), 7.71 (1H, t, J=1 Hz), 7.91 (1H, dt, J=0.5 Hz, 8 Hz).

LC/MS t=4.34 min.

### c) 3-{2-[5-Chloro-2-(4-chloro-benzyloxy)-phenyl]-5-methyl-pyrrol-1-yl}-benzoic acid

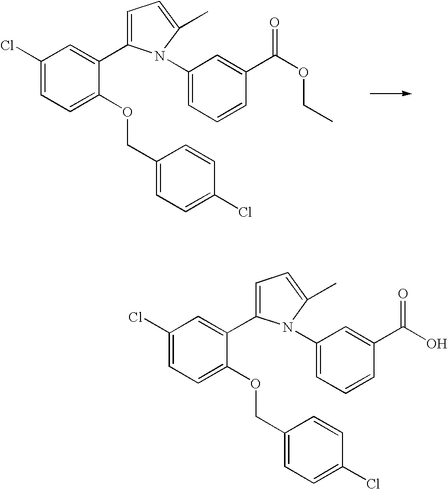

3-{2-[5-Chloro-2-(4-chloro-benzyloxy)-phenyl]-5-methyl-pyrrol-1-yl}-benzoic acid ethyl ester (0.102g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with EtOAc. The combined organic extracts were washed with brine and dried over MgSO4, filtered and concentrated in vacuo, to yield a white solid (0.075g, 78%).

¹H-NMR (400 MHz, d6DMSO) 2.08 (3H, s), 4.82 (2H, s), 6.08 (1H, dd, J=0.5 Hz, J=3 Hz), 6.27 (1H, d, J=3 Hz), 6.83 (1H, d, J=9 Hz), 7.10 (1H, d, J=3 Hz), 7.12 (2H, d, J=9 Hz), 7.17 (1H, dd, J=3 Hz, 9 Hz), 7.28-7.31 (1H, m), 7.38 (2H, d, J=9 Hz), 7.46 (1H, t, J=8 Hz), 7.54 (1H, t, J=0.5 Hz) 7.88 (1H, dt, J=0.5 Hz, 8 Hz).

LC/MS t=4.10 min [MH⁻] 450/452/454.

### Example 22

### 3-{2-[5-Chloro-2-(3,4-dichloro-benzyloxy)-phenyl]-5-methyl-pyrrol-1-yl}-benzoic acid

### a) 3-{2-[5-Chloro-2-(3,4-dichloro-benzyloxy)-phenyl]-5-methyl-pyrrol-1-yl}-benzoic acid ethyl ester

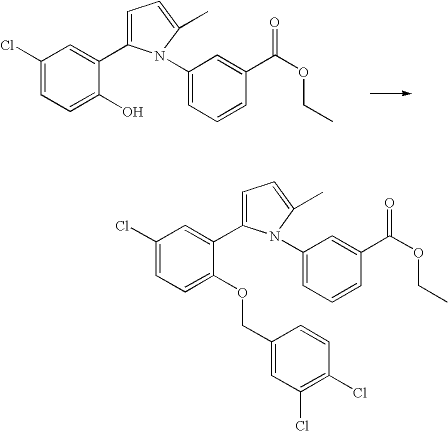

3-{2-[5-Chloro-2-(hydroxy)-phenyl]-5-methyl-pyrrol-1-yl}-benzoic acid ethyl ester (0.148g, 0.41 mmol), 3,4-dichlorobenzyl bromide (0.107 ml, 0.62 mmol) and potassium carbonate (0.12g, 0.82 mmol) were heated in DMF at 60° C. in a nitrogen atmosphere for 18 hours. Upon cooling the mixture was diluted with EtOAc and water. The organic layer was extracted and the aqueous layer washed with EtOAc. The combined organics were then washed with brine and concentrated in vacuo. This yielded the title compound as a clear oil (0.151 g, 70.6%).The residue was purified by chromatography on silica gel eluting with 5% EtOAc/iso-hexane.

¹H-NMR (400 MHz, CDCl₃) 1.31 (3H, t, 7 Hz), 2.17 (3H, s), 4.29 (2H, q, 7 Hz), 4.67 (2H, s), 6.15 (1H, dd, J=0.5 Hz, 3 Hz), 6.30 (1H, d, J=3 Hz), 6.50 (1H, d, J=9 Hz), 6.87 (1H, dd, J=2 Hz, 8 Hz), 7.05 (1H, dd, J=2 Hz, 9 Hz), 7.10-7.15 (2H, m), 7.26 (1H, d, J=2 Hz), 7.29 (1H, t, J=8 Hz), 7.35 (1H, d, J=8 Hz), 7.72 (1H, t, J=1 Hz), 7.91 (1H, dt, J=0.5 Hz, 8 Hz).

LC/MS t=4.45 min [MH⁺] 515/517.

### b) 3-{2-[5-Chloro-2-(3,4-dichloro-benzyloxy)-phenyl]-5-methyl-pyrrol-1-yl}-benzoic acid

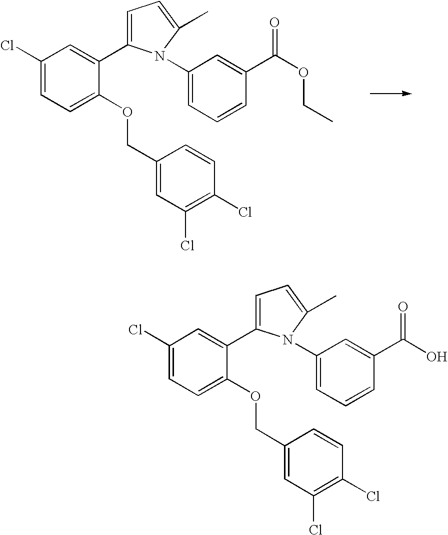

3-{2-[5-Chloro-2-(3,4-dichloro-benzyloxy)-phenyl]-5-methyl-pyrrol-1-yl}-benzoic acid ethyl ester (0.141g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with EtOAc. The combined organic extracts were washed with brine and dried over MgSO₄, filtered and concentrated in vacuo, to yield a yellow oil.

¹H-NMR (400 MHz, CDCl₃) 2.18 (3H, s), 4.66 (2H, s), 6.16 (1H, dd, J=0.5 Hz, J=3 Hz), 6.31 (1H, d, J=3 Hz), 6.52 (1H, d, J=9 Hz), 6.89 (1H, dd, J=2 Hz, 8 Hz), 7.08 (1H, dd, J=2 Hz, 9 Hz), 7.14 (1H, d, J=2 Hz), 7.15-7.19 (1H, m), 7.27 (1H, m′ excess), 7.33 (1H, t, J=8 Hz), 7.35 (1H, d, J=8 Hz), 7.77 (1H, t, J=0.5 Hz), 7.98 (1H, dt, J=0.5 Hz, 8 Hz).

LC/MS t=4.25 min [MH⁺] 487/489.

### Example 23

### 3-{2-[5-Chloro-2-(2-chloro-4-fluoro-benzyloxy)-phenyl]-5-methyl-pyrrol-1-yl}-benzoic acid

### a) 3-{2-[5-Chloro-2-(2-chloro-4-fluoro-benzyloxy)-phenyl]-5-methyl-pyrrol-1-yl}-benzoic acid ethyl ester

3-{2-[5-Chloro-2-(hydroxy)-phenyl]-5-methyl-pyrrol-1-yl}-benzoic acid ethyl ester (0.148g, 0.41 mmol), 2-chloro-4-fluoro-benzyl bromide (0.139g, 0.62 mmol) and potassium carbonate (0.1 2g, 0.82 mmol) were heated in DMF at 60° C. in a nitrogen atmosphere for 18 hours. Upon cooling the mixture was diluted with EtOAc and water. The organic layer was extracted and the aqueous layer washed with EtOAc. The combined organics were then washed with brine and concentrated in vacuo. This yielded the title compound as a clear oil (0.149g, 72%).The residue was purified by chromatography on silica gel eluting with 5% EtOAc/iso-hexane.

¹H-NMR (400 MHz, CDCl₃) 1.31 (3H, t, 7 Hz), 2.17 (3H, s), 4.29 (2H, q, 7 Hz), 4.73 (2H, s), 6.13 (1H, dd, J=0.5 Hz, 3 Hz), 6.31 (1H, d, 3 Hz), 6.52 (1H, d, 9 Hz), 6.85-6.91 (2H, m), 7.05-7.10 (2H, m), 7.15 (1H, m), 7.25 (1H, d, J=3 Hz), 7.30 (1H, t, J=8 Hz), 7.74 (1H, t, J=0.5 Hz), 7.92 (1H, dt, J=0.5 Hz, 8 Hz).

LC/MS t=4.36 min [MH⁺] 498/500/502.

### b) 3-{2-[5-Chloro-2-(2-chloro-4-fluoro-benzyloxy)-phenyl]-5-methyl-pyrrol-1-yl}-benzoic acid

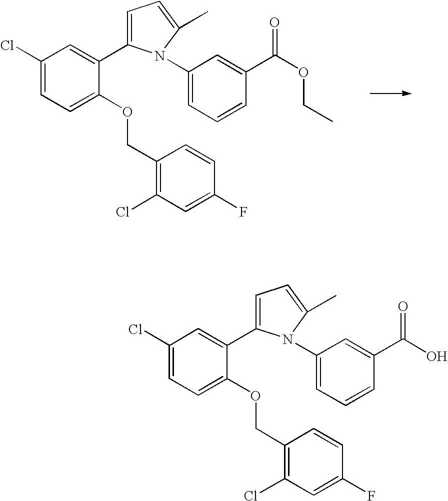

3-{2-[5-Chloro-2-(2-chloro-4-fluoro-benzyloxy)-phenyl]-5-methyl-pyrrol-1-yl}-benzoic acid ethyl ester (0.139g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with EtOAc. The combined organic extracts were washed with brine and dried over MgSO₄, filtered and concentrated in vacuo, to yield a yellow oil.

¹H-NMR (400 MHz, CDCl₃) 2.18 (3H, s), 4.75 (2H, s), 6.16 (1H, d, J=3 Hz), 6.31 (1H, d, J=3 Hz), 6.56 (1H, d, J=9 Hz), 6.87-6.97 (2H, m), 7.03-7.10 (2H, m), 7.17-7.21 (1H, m), 7.24 (1H, d, J=3 Hz), 7.34 (1H, t, J=8 Hz), 7.79 (1H, t, J=0.5 Hz), 7.99 (1H, dt, J=0.5 Hz, 8 Hz).

LC/MS t=4.13 min [MH⁺] 470/472/474.

### Example 24

### 3-{2-[5-Chloro-2-(4-fluoro-benzyloxy)-Phenyl]-5-methyl-pyrrol-1-yl}-benzoic acid

### a) 3-{2-[5-Chloro-2-(4-fluoro-benzyloxy)-phenyl]-5-methyl-pyrrol-1-yl}-benzoic acid ethyl ester

3-{2-[5-Chloro-2-(hydroxy)-phenyl]-5-methyl-pyrrol-1-yl}-benzoic acid ethyl ester (0.148g, 0.41 mmol), 4-fluorobenzyl bromide (0.078 ml, 0.62 mmol) and potassium carbonate (0.12g, 0.82 mmol) were heated in DMF at 60° C. in a nitrogen atmosphere for 18 hours. Upon cooling the mixture was diluted with EtOAc and water. The organic layer was extracted and the aqueous layer washed with EtOAc. The combined organics were then washed with brine and concentrated in vacuo. This yielded the title compound as a clear oil (0.142g, 74%).The residue was purified by chromatography on silica gel eluting with 5% EtOAc/iso-hexane.

¹H-NMR (400 MHz, CDCl₃) 1.31 (3H, t, 7 Hz), 2.16 (3H, s), 4.29 (2H, q, 7 Hz), 4.68 (2H, s), 6.13 (1H, d, J=3 Hz), 6.30 (1H, d, 3 Hz), 6.54 (1H, d, 9 Hz), 6.96-7.06 (5H, m), 7.10 (1H, dt, J=0.5 Hz, J=8 Hz), 7.23 (1H, d, J=3 Hz), 7.28 (1H, t, J=8 Hz), 7.70 (1H, t, J=0.5 Hz), 7.90 (1H, dt, J=0.5 Hz, 8 Hz).

LC/MS t=4.22 min [MH⁺] 464/466.

### b) 3-{2-[5-Chloro-2-(4-fluoro-benzyloxy)-phenyl]-5-methyl-pyrrol-1-yl}-benzoic acid

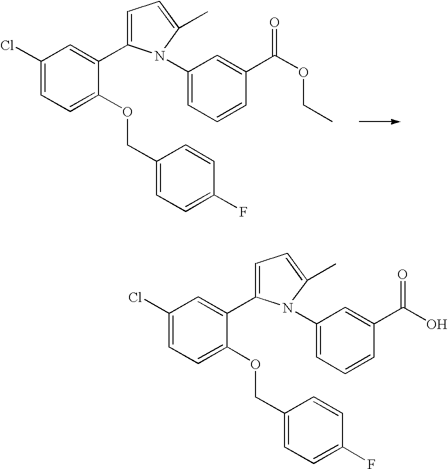

3-{2-[5-Chloro-2-(4-fluoro-benzyloxy)-phenyl]-5-methyl-pyrrol-1-yl}-benzoic acid ethyl ester (0.132g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with EtOAc. The combined organic extracts were washed with brine and dried over MgSO₄, filtered and concentrated in vacuo, to yield a yellow solid (0.105g, 85%).

¹H-NMR (400 MHz, DMSO) 2.08 (3H, s), 4.80 (2H, s), 6.07 (1H, dd, J=0.5 Hz, 3 Hz), 6.24 (1H, d, J=3 Hz), 6.89 (1H, d, J=9 Hz), 7.09 (1H, d, J=3 Hz), 7.10-7.20 (5H, m), 7.25-7.29 (1H, m), 7.45 (1H, t, J=8 Hz), 7.52 (1H, t, J=0.5 Hz), 7.87 (1H, dt, J=0.5 Hz, 8 Hz).

LC/MS t=3.96 min [MH⁺] 436/438.

### Example 26

### 3-{2-[6-Chloro-2-(2,4-difluoro-benzyloxy)-phenyl]-5-methyl-pyrrol-1-yl}-benzoic acid

### a) 3-{2-[5-Chloro-2-(2,4-difluoro-benzyloxy)-phenyl]-5-methyl-pyrrol-1-yl}-benzoic acid ethyl ester

3-{2-[5-Chloro-2-(hydroxy)-phenyl]-5-methyl-pyrrol-1-yl}-benzoic acid ethyl ester (0.148g, 0.41 mmol), 2,4-difluorobenzyl bromide (0.078 ml, 0.62 mmol) and potassium carbonate 0.12g, 0.82 mmol) were heated in DMF at 60° C. in a nitrogen atmosphere for 18 hours. Upon cooling the mixture was diluted with EtOAc and water. The organic layer was extracted and the aqueous layer washed with EtOAc. The combined organics were then washed with brine and concentrated in vacuo. This yielded the title compound as a clear oil (0.143g, 71%).The residue was purified by chromatography on silica gel eluting with 5% EtOAc/iso-hexane.

¹H-NMR (400 MHz, CDCl₃) 1.31 (3H, t, 7 Hz), 2.16 (3H, s), 4.30 (2H, q, 7 Hz), 4.73 (2H, s), 6.13 (1H, dd, J=0.5 Hz, J=3 Hz), 6.30 (1H, d, 3 Hz), 6.59 (1H, d, 9 Hz), 6.76-6.81 (2H, m), 6.93-6.99 (1H, m,), 7.07 (1H, dd, J=1.5 Hz, 9 Hz) 7.13 (1H, m), 7.21 (1H, d, J=3 Hz), 7.29 (1H, t, J=8 Hz), 7.72 (1H, t, J=1 Hz), 7.91 (1H, dt, J=0.5 Hz, 8 Hz).

LC/MS t=4.24 min [MH⁺] 482/484.

### b) 3-{2-[5-Chloro-2-(2,4-difluoro-benzyloxy)-phenyl]-5-methyl-pyrrol-1-yl}-benzoic acid

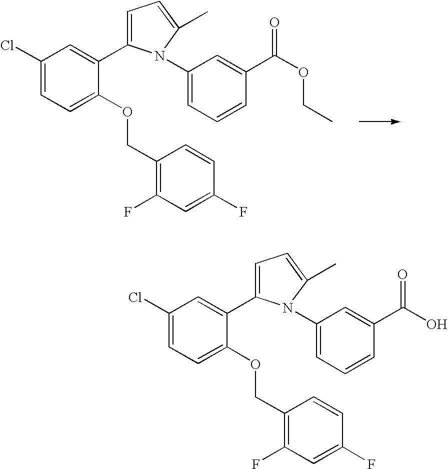

3-{2-[5-Chloro-2-(2,4-difluoro-benzyloxy)-phenyl]-5-methyl-pyrrol-1-yl}-benzoic acid ethyl ester (0.1 33g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with EtOAc. The combined organic extracts were washed with brine and dried over MgSO₄, filtered and concentrated in vacuo, to yield a yellow solid (0.103g, 82%).

¹H-NMR (400 MHz, d6-DMSO) 2.08 (3H,s), 4.84 (2H, s), 6.07 (1H, d, J=3 Hz), 6.26 (1H, d, J=3 Hz), 6.97 (1H, d, J=9 Hz), 7.07 (1H, ddd, J=2 Hz, 9 Hz), 7.09 (1H, d, J=3 Hz), 7.18-7.30 (4H, m), 7.46 (1H, t, J=8 Hz), 7.52 (1H, t, J=0.5 Hz), 7.87 (1H, dt, J=0.5 Hz, 8 Hz).

LC/MS t=3.98 min [MH⁺] 454/456.

### Example 26

### 5-{2-[2-(4-Methoxy-benzyloxy)-phenyl-6-methyl-pyrrol-1-yl}-nicotinic acid

### a) 5-Amino-nicotinic acid

Copper (II) sulfate (12.5g, 50 mmol) was added to 5-bromo-nicotinic acid (50g, 248 mmol) in aqueous ammonium hydroxide solution (d=0.88). The reaction was sealed in an autoclave reactor and heated at 1 80° C. for 15 hours. The mixture was cooled, diluted with water (300 ml), sodium sulfite (13.5g, 173 mmol) was added and the mixture stirred for 20 minutes. The black precipitate was filtered away through celite, and the filtrate was adjusted to pH 3-4 upon treatment with 2M HCl. The mixture was filtered through celite and the filtrate concentrated to 200 ml volume upon which a white precipitate formed. The solution was cooled, filtered and the solid dried under vacuum at 40° C. overnight to give the title compound (22g, 159 mmol, 64%).

¹NMR (400 MHz, d6-DMSO) 5.60 (2H, broad s), 7.41 (1H, d, J=3 Hz), 8.10 (1H, d, J=3 Hz), 8.24 (1H, d, J=3 Hz), 13.05 (1H, broad s).

GC/MS [MH⁺] 139.

### b) 5-Amino-nicotinic acid ethyl ester

HCl (4M in dioxane, 100 ml, 0.4 mol) was added to 5-Amino-nicotinic acid (22g, 159 mmol) in ethanol (500 ml) and heated at reflux for 16 hours. The reaction was cooled, concentrated and partitioned between ethyl acetate and saturated NaHCO₃. The aqueous was extracted with EtOAc and the combined organics washed with brine, dried (MgSO₄), filtered and concentrated to give the title compound (22g, 159 mmol, 83%).

¹H NMR (400 MHz, CDCl₃) 1.40 (3H, t, J=7 Hz), 3.83 (2H, broad s), 4.39 (2H, q, J=7 Hz), 7.57 (1H, dd, J=2, 3 Hz), 8.23 (1H, d, J=3 Hz), 8.63 (1H, d, J=2 Hz).

LC/MS t=1.48 min [MH+] 167.

### c) 5-{2-[2-(4-Methoxy-benzyloxy)-phenyl]-5-methyl-pyrrol-1-yl}-nicotinic acid ethyl ester

1-[2-(4-Methoxy-benzyloxy)-phenyl]-pentane-1,4-dione (1.05g, 3.3 mmol) and 5-amino-nicotinic acid ethyl ester (0.6g, 3.7 mmol) were heated in toluene (1 ml) in a sealed vessel at 150° C. for 3 days. Upon cooling, the mixture was diluted with EtOAc and washed with 2M HCl and saturated NaHCO₃, dried (MgSO₄), filtered and concentrated. The residue was purified by chromatography on silica gel with isohexane/EtOAc (10%) as eluant, to give the title compound (530 mg, 36%).

¹H NMR (400 MHz, CDCl₃) 1.33 (3H, t, J=7.5 Hz), 2.16 (3H, s), 3.81 (3H, s), 4.32 (2H, q, J=7.5 Hz), 4.67 (2H, s), 6.16 (1H, d, J=3 Hz), 6.30 (1H, d, J=3 Hz), 6.69 (1H, broad d, J=8.5 Hz), 6.83 (2H, d, J=9 Hz), 6.91 (1H, ddd, J=, 7 Hz), 7.02 (2H, d, J=9 Hz), 7.15 (1H, ddd, J=2, 7 Hz), 7.29 (1H, dd, J=2, 8.5 Hz), 7.88 (1H, t, J=2 Hz), 8.35 (1H, d, J=2 Hz), 9.01 (1H, d, J=2 Hz).

LC/MS t=3.80 min [MH+] 443.

### d) 5-{2-[2-(4-Methoxy-benzyloxy)-phenyl]-5-methyl-pyrrol-1-yl}-nicotinic acid

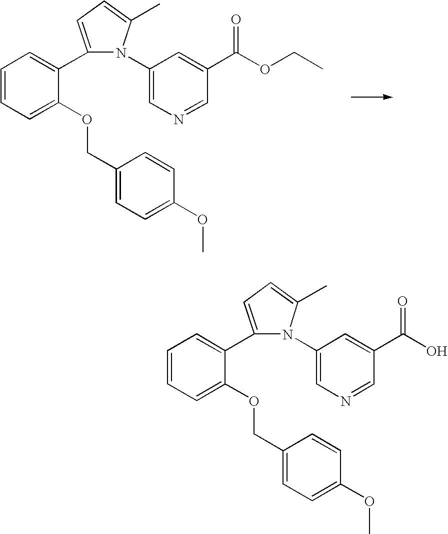

5-{2-[2-(4-Methoxy-benzyloxy)-phenyl]-5-methyl-pyrrol-1-yl}-nicotinic acid ethyl ester (70 mg, 0.16 mmol) was heated at reflux in a 1:1 mixture of ethanol/2M NaOH (3 ml) in a sealed vessel for 1.5 hours. Upon cooling, the reaction was diluted with ethyl acetate, washed with 2M HCl and brine, dried (MgSO₄) and concentrated to give the title compound (57 mg, 87%).

¹H NMR (400 MHz, CDCl₃) 2.17 (3H, s), 3.76 (3H, s), 4.65 (2H, s), 6.18 (1H, dd, J=1, 3.5 Hz), 6.30 (1H, d, J=3.5 Hz), 6.71 (1H, broad d, J=8.5 Hz), 6.82 (2H, d, J=9 Hz), 6.93 (1H, ddd, J=1, 8 Hz), 7.02 (2H, d, J=9 Hz), 7.16 (1H, ddd, J=2, 8 Hz), 7.31 (1H, dd, J=2, 8 Hz), 7.92 (1H, t, J=2 Hz), 8.38 (1H, d, J=2 Hz), 9.08 (1H, d, J=2 Hz).

LC/MS t=3.63 min [MH−] 413.

### Example 27

### 5-{2-[2-(4-Chloro-benzyloxy)-phenyl]-5-methyl-pyrrol-1-yl}-nicotinic acid

### a) 5-{2-[2-(Hydroxy)-phenyl]-5-methyl-pyrrol-1-yl}-nicotinic acid ethyl ester

HCl (4M in dioxane, 2.5 ml, 1 0 mmol) was added to 5-{2-[2-(4-Methoxy-benzyloxy)-phenyl]-5-methyl-pyrrol-1-yl}-nicotinic acid ethyl ester (460 mg, 1 mmol) and stirred at room temperature for 1 hour. The reaction was concentrated and the residue partitioned between CH₂Cl₂ and NaHCO₃. The organics were washed with brine, dried (MgSO₄), filtered and concentrated. The residue was purified by chromatography, with isohexane/EtOAc (30%) as eluant, to give the title compound (200 mg, 67%).

¹H NMR (400 MHz, CDCl₃) 1.38 (3H, t, J=7 Hz), 2.18 (3H, s), 4.39 (2H, q, J=7 Hz), 6.21 (1H, broad d, J=3 Hz), 6.39 (1H, d, J=3 Hz), 6.67 (1H, ddd, J=1, 8 Hz), 6.74 (1H, dd, J=2, 8 Hz), 7.10 (1H, ddd, J=2, 8 Hz), 7.38 (1H, dd, J=3, 8.5 Hz), 8.04 (1H, t, J=2 Hz), 8.48 (1H, d, J=2 Hz), 9.09 (1H, d, J=2 Hz).

LC/MS t=3.25 min [MH+] 323.

### b) 5-{2-[2-(4-Chloro-benzyloxy)-phenyl]-5-methyl-pyrrol-1-yl}-nicotinic acid ethyl ester

4-Chloro-benzyl bromide (48 mg, 0.23 mmol) was added to 5-{2-[2-(Hydroxy)-phenyl]-5-methyl-pyrrol-1-yl}-nicotinic acid ethyl ester (50 mg, 0.16 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (15 mg, 21%).

¹H NMR (400 MHz, CDCl₃) F4743 1.33 (3H, t, J=7 Hz), 2.17 (3H, s), 4.32 (2H, q, J=7 Hz), 4.70 (2H, s), 6.17 (1H, dd, J=1, 3 Hz), 6.30 (1H, d, J=3 Hz), 6.64 (1H, broad d, J=8 Hz), 6.93 (1H, ddd, J=1, 8 Hz), 7.01 (2H, d, J=1 Hz), 7.15 (1H, ddd, J=2, 8 Hz), 7.16 (1H, ddd, J=2, 8 Hz), 7.27 (2H, d, J=8 Hz), 7.30 (1H, dd, J=2, 8 Hz), 7.89 (1H, t, J=2 Hz), 8.35 (1H, d, 2 Hz), 9.02 (1H, broad s).

LC/MS t=3.98 min [MH+] 447/449.

### c) 5-{2-[2-(4-Chloro-benzyloxy)-phenyl]-5-methyl-pyrrol-1-yl}-nicotinic acid

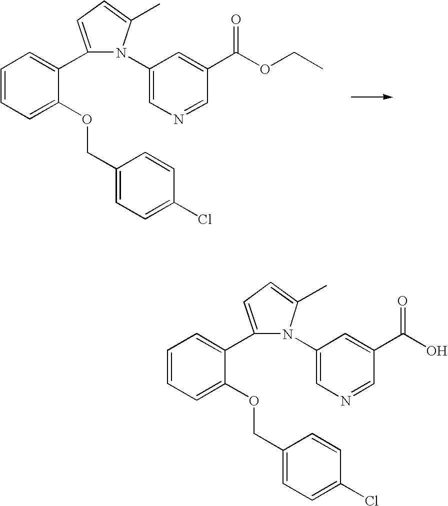

5-{2-[2-(4-Chloro-benzyloxy)-phenyl]-5-methyl-pyrrol-1-yl}-nicotinic acid ethyl ester (1 5 mg, 0.03 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (10 mg, 71%).

¹H NMR (400 MHz, CDCl₃) 2.18 (3H, s), 4.69 (2H, s), 6.18 (1H, broad d, J=3.5 Hz), 6.31 (1H, d, J=3.5 Hz), 6.65 (1H, broad d, J=8 Hz), 6.94 (1H, broad t, J=8 Hz), 7.12 (2H, d, J=8 Hz), 7.16 (1H, ddd, J=2, 8 Hz), 7.28 (2H, d, J=8 Hz), 7.31 (1H, dd, J=2, 8 Hz), 7.90 (1H, t, J=2 Hz), 8.39 (1H, broad s), 9.06 (1H, broad s).

LC/MS t=3.85 min [MH+] 419/421.

### Example 28

### 5-{2-[2-(3,4-Dichloro-benzyloxy)-phenyl]-5-methyl-pyrrol-1-yl}-nicotinic acid

### a) 5-{2-[2-(3,4-Dichloro-benzyloxy)-phenyl]-5-methyl-pyrrol-1-yl}-nicotinic acid ethyl ester

3,4-Dichloro-benzyl bromide (0.04 ml, 0.23 mmol) was added to 5-{2-[2-(Hydroxy)-phenyl]-5-methyl-pyrrol-1-yl}-nicotinic acid ethyl ester (50 mg, 0.16 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (1 3 mg, 17%).

¹H NMR (400 MHz, CDCl₃) 1.32 (3H, t, J=7 Hz), 2.18 (3H, s), 4.31 (2H, q, J=7 Hz), 4.68 (2H, s), 6.19 (1H, dd, J=1, 3 Hz), 6.30 (1H, d, J=3 Hz), 6.62 (1H, dd, J=8 Hz), 6.87-6.98 (2H, m), 7.12-7.22 (2H, m), 7.28-7.38 (2H, m), 7.91 (1H, t, J=2 Hz), 8.38 (1H, d, 2 Hz), 9.02 (1H, broad s).

LC/MS t=4.10 min [MH+] 481/483/485.

### b) 5-{2-[2-(3,4-Dichloro-benzyloxy)-phenyl]-5-methyl-pyrrol-1-yl}-nicotinic acid

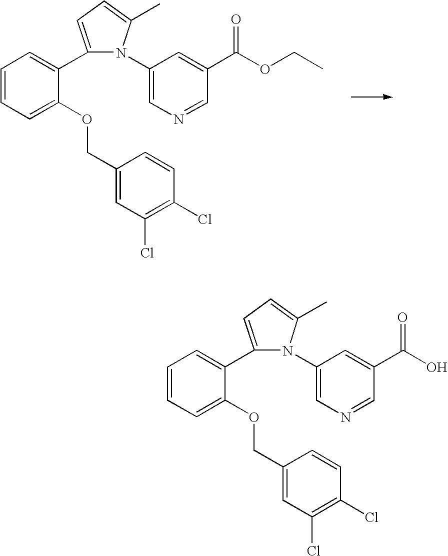

5-{2-[2-(3,4-Dichloro-benzyloxy)-phenyl]-5-methyl-pyrrol-1-yl}-nicotinic acid ethyl ester (13 mg, 0.03 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (10 mg, 82%).

¹H NMR (400 MHz, CDCl₃) 2.20 (3H, s), 4.68 (2H, s), 6.19 (1H, dd, J=1, 3 Hz), 6.20 (1H, d, J=3 Hz), 6.32 (1H, dd, J=4 Hz), 6.64 (1H, d, J=8 Hz), 6.90-7.00 (2H, m), 7.13-7.20 (2H, m), 7.33 (1H, dd, J=2, 8 Hz), 7.37 (1H, d, J=8 Hz), 7.92 (1H, t, J=2 Hz), 8.42 (1H, d, 2 Hz), 9.07 (1H, d, J=2 Hz).

LC/MS t=4.06 min [MH+] 453/455/457.

### Example 29

### 5-{2-[2-(4-Fluoro-benzyloxy)-phenyl]-5-methyl-pyrrol-1-yl]-nicotinic acid

### a) 5-{2-[2-(4-Fluoro-benzyloxy)-phenyl]-5-methyl-pyrrol-1-yl}-nicotinic acid ethyl ester

4-Fluoro-benzyl bromide (0.029 ml, 0.23 mmol) was added to 5-{2-[2-(Hydroxy)-phenyl]-5-methyl-pyrrol-1-yl}-nicotinic acid ethyl ester (50 mg, 0.16 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO4), filtered and concentrated. The residue was purified by chromatography on silica gel with isohexane/EtOAc (10%) as eluant, to give the title compound (20 mg, 30%).

¹H NMR (400 MHz, CDCl₃) 1.33 (3H, t, J=7 Hz), 2.16 (3H, s), 4.31 (2H, q, J=7 Hz), 4.69 (2H, s), 6.17 (1H, broad d, J=3.5 Hz), 6.30 (1H, d, J=3.5 Hz), 6.67 (1H, broad d, J=8 Hz), 6.90-7.08 (5H, m), 7.15 (1H, ddd, J=2, 8 Hz), 7.29 (1H, dd, J=2, 8 Hz), 7.87 (1H, t, J=2 Hz), 8.34 (1H, d, 2 Hz), 9.01 (1H, d, J=2 Hz).

LC/MS t=3.84 min [MH+] 431

### b) 5-{2-[2-(4-Fluoro-benzyloxy)-phenyl]-5-methyl-pyrrol-1-yl}-nicotinic acid

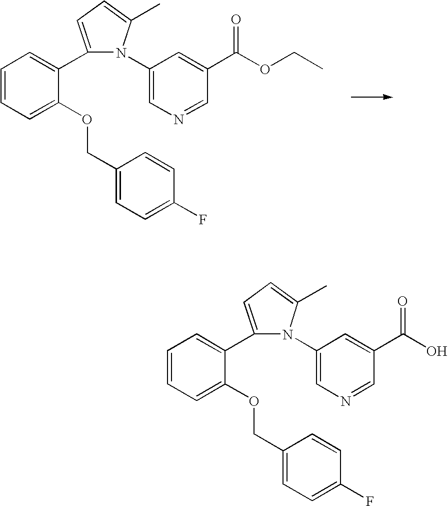

5-{2-[2-(4-Fluoro-benzyloxy)-phenyl]-5-methyl-pyrrol-1-yl}-nicotinic acid ethyl ester (1 6 mg, 0.04 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (13 mg, 87%).

¹H NMR (400 MHz, CDCl₃) 2.17 (3H, s), 4.68 (2H, s), 6.18 (1H, broad d, J=3 Hz), 6.31 (1H, d, J=3 Hz), 6.68 (1H, broad d, J=8 Hz), 6.92-7.02 (3H, m), 7.03-7.10 (2H, m), 7.17 (1H, ddd, J=2, 8 Hz), 7.32 (1H, dd, J=2, 8 Hz), 7.90 (1H, broad s), 8.38 (1H, broad s), 9.07 (1H, broad s).

LC/MS t=3.65 min [MH+] 403.

### Example 30

### 5-{2-[2-(2,4-Difluoro-benzyloxy)-phenyl]-5-methyl-pyrrol-1-yl}-nicotinic acid

### a) 5-{2-[2-(2,4-Difluoro-benzyloxy)-phenyl]-5-methyl-pyrrol-1-yl}-nicotinic acid ethyl ester

2,4-Difluorobenzyl bromide (0.030 ml, 0.23 mmol) was added to 5-{2-[2-(Hydroxy)-phenyl]-5-methyl-pyrrol-1-yl}-nicotinic acid ethyl ester (50 mg, 0.16 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (19 mg, 27%).

¹H NMR (400 MHz, CDCl₃) 1.34 (3H, t, J=7 Hz), 2.17 (3H, s), 4.33 (2H, q, J=7 Hz), 4.76 (2H, s), 6.17 (1H, dd, J=1, 3.5 Hz), 6.29 (1H, d, J=3.5 Hz), 6.70 (1H, broad d, J=8 Hz), 6.74-6.84 (2H, m), 6.93 (1H, ddd, J=1, 8 Hz), 6.97-7.06(1H, m), 7.17 (1H, ddd, J=2,8 Hz), 7.27 (1H, dd, J=2, 8 Hz), 7.91 (1H, t, J=2 Hz), 8.37 (1H, broad d, 2 Hz), 9.03 (1H, broad s).

LC/MS t=3.86 min [MH+] 449.

### b) 5-{2-[2-(2,4-Difluoro-benzyloxy)-phenyl]-5-methyl-pyrrol-1-yl}-nicotinic acid

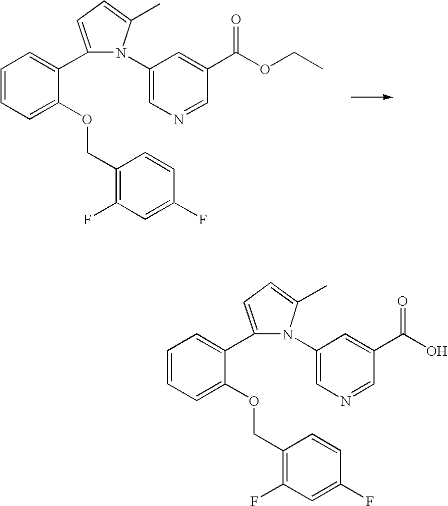

5-{2-[2-(2,4-Difluoro-benzyloxy)-phenyl]-5-methyl-pyrrol-1-yl}-nicotinic acid ethyl ester (13 mg, 0.03 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (10 mg, 82%).

¹H NMR (400 MHz, CDCl₃) 2.18 (3H, s), 4.75 (2H, s), 6.18 (1H, broad d, J=3 Hz), 6.30 (1H, d, J=3 Hz), 6.68-6.85 (3H, m), 6.92-7.09 (3H, m), 7.19 (1H, broad ddd, J=8 Hz), 7.30 (1H, dd, J=2, 8 Hz), 7.96 (1H, broad s), 8.42 (1H, broad s), 9.10 (1H, broad s).

LC/MS t=3.66 min [MH+] 421.

### Example 31

### 5-{2-[5-Chloro-2-(4-methoxy-benzyloxy)-phenyl]-5-methyl-pyrrol-1-yl}-nicotinic acid

### a) 5-{2-[5-Chloro-2-(4-methoxy-benzyloxy)-phenyl]-5-methyl-pyrrol-1-yl}-nicotinic acid ethyl ester

1-[5-Chloro-2-(4-methoxy-benzyloxy)-phenyl]-pentane-1,4dione (1.04g, 3.0 mmol) and 5-amino-nicotinic acid ethyl ester (0.55g, 3.3 mmol) were heated in toluene (0.5 ml) in a sealed vessel at 150° C. for 3 days. Upon cooling, the mixture was diluted with EtOAc and washed with 2M HCl and saturated NaHCO₃, dried (MgSO₄), filtered and concentrated. The residue was purified by chromatography on silica gel with isohexane containing a gradient of EtOAc (15-20%) as eluant, to give the title compound (740 mg, 52%).

¹H NMR (400 MHz, CDCl₃) 1.34 (3H, t, J=7.5 Hz), 2.15 (3H, s), 3.80 (3H, s), 4.33 (2H, q, J=7.5 Hz), 4.61 (2H, s), 6.16 (1H, d, J=3 Hz), 6.29 (1H, d, J=3 Hz), 6.60 (1H, broad d, J=9 Hz), 6.83 (2H, d, J=9 Hz), 6.97 (2H, d, J=9 Hz), 7.09 (1H, dd, J=2.5, 9 Hz), 7.29 (1H, d, J=2.5 Hz), 7.87 (1H, t, J=2 Hz), 8.35 (1H, d, J=2 Hz), 9.04 (1H, d, J=2 Hz).

LC/MS t=3.96 min [MH+] 477/479.

### b) 5-{2-[5-Chloro-2-(4-methoxy-benzyloxy)-phenyl]-5-methyl-pyrrol-1-yl}-nicotinic acid

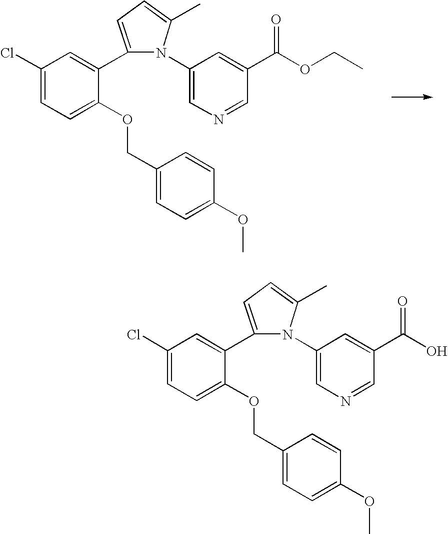

5-{2-[5-Chloro-2-(4-methoxy-benzyloxy)-phenyl]-5-methyl-pyrrol-1-yl}-nicotinic acid ethyl ester (90 mg, 0.16 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (75 mg, 89%).

¹H NMR (400 MHz, CDCl₃) 2.15 (3H, s), 3.79 (3H, s), 4.61 (2H, s), 6.17 (1H, dd, J=1, 4 Hz), 6.30 (1H, d, J=4 Hz), 6.62 (1H, d, J=9 Hz), 6.83 (2H, d, J=9 Hz), 6.98 (2H, d, J=9 Hz), 7.10 (1H, dd, J=3, 9 Hz), 7.31 (1H, d, J=3 Hz), 7.89 (1H, t, J=2 Hz), 8.37 (1H, d, J=2 Hz), 9.08 (1H, d, J=2 Hz).

LC/MS t=3.81 min [MH−] 447/449.

### Example 32

### 5-{2-[5-Chloro-2-(benzyloxy)-phenyl]-5-methyl-pyrrol-1-yl}-nicotinic acid

### a) 5-{2-[5-Chloro-2-(hydroxy)-phenyl]-5-methyl-pyrrol-1-yl}-nicotinic acid ethyl ester

HCl (4M in dioxane, 2.5 ml, 10 mmol) was added to 5-{2-[5-Chloro-2-(4-methoxy-benzyloxy)-phenyl]-5-methyl-pyrrol-1-yl}-nicotinic acid ethyl ester (640 mg, 1.3 mmol) and stirred at room temperature for 30 minutes. The reaction was concentrated and the residue partitioned between EtOAc and NaHCO₃. The organics were washed with brine, dried (MgSO₄), filtered and concentrated. The residue was purified by chromatography on silica gel with isohexane/EtOAc (20%) as eluant, to give the title compound (320 mg, 67%).

¹H NMR (400 MHz, CDCl₃) 1.39 (3H, t, J=7 Hz), 2.17 (3H, s), 4.39 (2H, q, J=7 Hz), 6.20 (1H, d, J=3.5 Hz), 6.37 (1H, d, J=3.5 Hz), 6.74 (1H, d, J=9 Hz), 6.87 (1H, d, J=2.5 Hz), 7.05 (1H, dd, J=2.5, 9 Hz), 8.04 (1H, t, J=2 Hz), 8.49 (1H, d, J=2 Hz), 9.06 (1H, d, J=2 Hz).

LC/MS t=3.48 min [MH+] 357/359.

### b) 5-[2-(5-Chloro-2-benzyloxy-phenyl)-5-methyl-pyrrol-1-yl]-nicotinic acid ethyl ester

Benzyl bromide (0.025 ml, 0.21 mmol) was added to 5-{2-[5-Chloro-2-(hydroxy)-phenyl]-5-methyl-pyrrol-1-yl}-nicotinic acid ethyl ester (50 mg, 0.14 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (15%) as eluant, to give the title compound (35 mg, 56%).

¹H NMR (400 MHz, CDCl₃) 1.34 (3H, t, J=7 Hz), 2.15 (3H, s), 4.34 (2H, q, J=7 Hz), 4.70 (2H, s), 6.17 (1H, d, J=3.5 Hz), 6.31 (1H, d, J=3.5 Hz), 6.58 (1H, d, J=9 Hz), 7.00-7.05 (2H, m), 7.09 (1H, dd, J=3, 9 Hz), 7.26-7.32 (4H, m), 7.90 (1H, t, J=2 Hz), 8.36 (1H, d, 2 Hz), 9.04 (1H, d, J=2 Hz).

LC/MS t=3.98 min [MH+] 447/449.

### c) 5-{2-[5-Chloro-2-(benzyloxy)-phenyl]-5-methyl-pyrrol-1-yl}-nicotinic acid

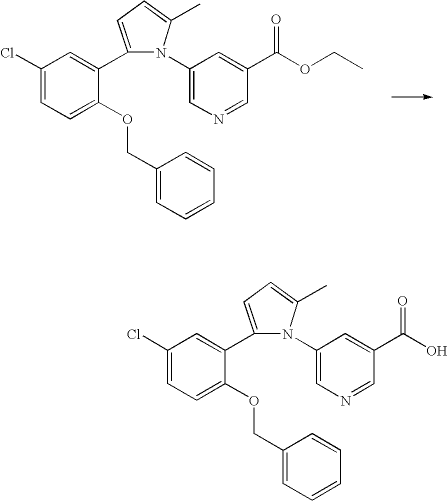

5-{2-[5-Chloro-2-(benzyloxy)-phenyl]-5-methyl-pyrrol-1-yl}-nicotinic acid ethyl ester (35 mg, 0.08 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (24 mg, 73%).

¹H NMR (400 MHz, d6-DMSO) 2.10 (3H, s), 4.78 (2H, s), 6.13 (1H, dd, J=1, 3.5 Hz), 6.28 (1H, d, J=3.5 Hz), 6.84 (1H, d, J=9 Hz), 7.05-7.11 (2H, m), 7.20-7.34 (5H, m), 7.83 (1H, t, J=2 Hz), 8.43 (1H, d, J=2 Hz), 8.95 (1H, d, J=2 Hz), 13.48 (1H, broad s).

LC/MS t=3.86 min [MH+] 419/421.

### Example 33

### 5-{2-[5-Chloro-2-(4-chloro-benzyloxy)-phenyl]-5-methyl-pyrrol-1-yl}-nicotinic acid

### a) 5-{2-[5-Chloro-2-(4-chloro-benzyloxy)-phenyl]-5-methyl-pyrrol-1-yl}-nicotinic acid ethyl ester

4-Chloro-benzyl bromide (43 mg, 0.21 mmol) was added to 5-{2-[5-Chloro-2-(hydroxy)-phenyl]-5-methyl-pyrrol-1-yl}-nicotinic acid ethyl ester (50 mg, 0.14 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (15%) as eluant, to give the title compound (30 mg, 44%).

¹H NMR (400 MHz, CDCl₃) 1.35 (3H, t, J=7 Hz), 2.16 (3H, s), 4.33 (2H, q, J=7 Hz), 4.65 (2H, s), 6.17 (1H, dd, J=1, 3.5 Hz), 6.30 (1H, d, J=3.5 Hz), 6.55 (1H, d, J=9 Hz), 6.97 (2H, d, J=8.5 Hz), 7.10 (1H, dd, J=2.5, 9 Hz), 7.27 (2H, d, J=8.5 Hz), 7.31 (1H, d, J=2.5 Hz), 7.88 (1H, t, J=2 Hz), 8.36 (1H, d, 2 Hz), 9.04 (1H, d, J=2 Hz).

LC/MS t=4.12 min [MH+] 481/483/485.

### b) 5-{2-[5-Chloro-2-(4-chloro-benzyloxy)-phenyl]-5-methyl-pyrrol-1-yl}-nicotinic acid

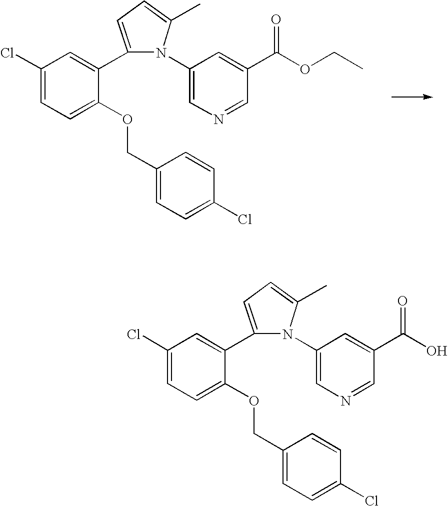

5-{2-[5-Chloro-2-(4-chloro-benzyloxy)-phenyl]-5-methyl-pyrrol-1-yl}-nicotinic acid ethyl ester (30 mg, 0.06 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (22 mg, 78%).

¹H NMR (400 MHz, d6-DMSO) 2.10 (3H, s), 4.77 (2H, s), 6.13 (1H, dd, J=1, 3.5 Hz), 6.28 (1H, d, J=3.5 Hz), 6.83 (1H, d, J=9 Hz), 7.10 (2H, d, J=8.5 Hz), 7.21-7.27 (2H, m), 7.37 (2H, d, J=8.5 Hz), 7.82 (1H, t, J=2 Hz), 8.43 (1H, d, J=2 Hz), 8.92 (1H, d, J=2 Hz), 13.43 (1H, broad s).

LC/MS t=4.08 min [MH+] 453/455/457.

### Example 34

### 5-{2-[5-Chloro-2-(4-fluoro-benzyloxy)-phenyl]-5-methyl-pyrrol-1-yl}-nicotinic acid

### a) 5-{2-[5-Chloro-2-(4-fluoro-benzyloxy)-phenyl]-5-methyl-pyrrol-1-yl}-nicotinic acid ethyl ester

4-Fluoro-benzyl bromide (0.027 ml, 0.21 mmol) was added to 5-{2-[5-Chloro-2-(hydroxy)-phenyl]-5-methyl-pyrrol-1-yl}-nicotinic acid ethyl ester (50 mg, 0.14 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (15%) as eluant, to give the title compound (35 mg, 54%).

¹H NMR (400 MHz, CDCl₃) 1.35 (3H, t, J=7 Hz), 2.15 (3H, s), 4.33 (2H, q, J=7 Hz), 4.64 (2H, s), 6.17 (1H, dd, J=1, 3.5 Hz), 6.30 (1H, d, J=3.5 Hz), 6.58 (1H, d, J=9 Hz), 6.96-7.05 (4H, m), 7.10 (1H, ddd, J=3, 9 Hz), 7.31 (1H, d, J=2.5 Hz), 7.86 (1H, t, J=2 Hz), 8.34 (1H, d, 2 Hz), 9.04 (1H, d, J=2 Hz).

LC/MS t=3.99 min [MH+] 465/467.

### b) 5-{2-[5-Chloro-2-(4-fluoro-benzyloxy)-phenyl]-5-methyl-pyrrol-1-yl}-nicotinic acid

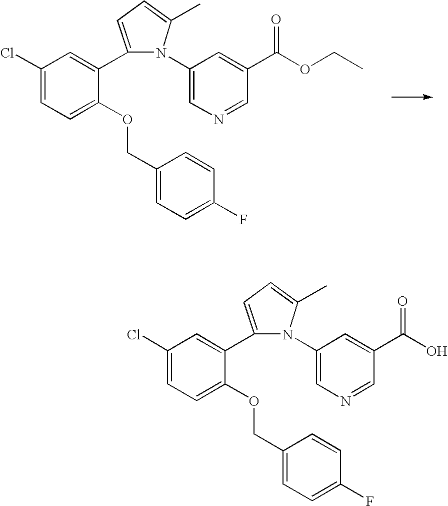

5-{2-[5-Chloro-2-(4-fluoro-benzyloxy)-phenyl]-5-methyl-pyrrol-1-yl}-nicotinic acid ethyl ester (35 mg, 0.08 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (25 mg, 76%).

¹H NMR (400 MHz, d6-DMSO) 2.08 (3H, s), 4.77 (2H, s), 6.11 (1H, broad d, J=3.5 Hz), 6.26 (1H, d, J=3.5 Hz), 6.87 (1H, d, J=9 Hz), 7.11-7.25 (6H, m), 7.81 (1H, broad s), 8.34 (1H, d, J=2 Hz), 8.92 (1H, d, J=2 Hz).

LC/MS t=3.87 min [MH+] 437/439.

### Example 35

### 5-{2-[5-Chloro-2-(2-chloro-4-fluoro-benzyloxy)-phenyl]-5-methyl-pyrrol-1-yl}-nicotinic acid

### a) 5-{2-[5-Chloro-2-(2-chloro-4-fluoro-benzyloxy)-phenyl]-5-methyl-pyrrol-1-yl}-nicotinic acid ethyl ester

2-Chloro-fluoro-benzyl bromide (47 mg, 0.21 mmol) was added to 5-{2-[5-Chloro-2-(hydroxy)-phenyl]-5-methyl-pyrrol-1-yl}-nicotinic acid ethyl ester (50 mg, 0.1 4 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (15%) as eluant, to give the title compound (39 mg, 56%).

¹H NMR (400 MHz, CDCl₃) 1.35 (3H, t, J=7 Hz), 2.17 (3H, s), 4.34 (2H, q, J=7 Hz), 4.73 (2H, s), 6.17 (1H, dd, J=1, 3.5 Hz), 6.32 (1H, d, J=3.5 Hz), 6.57 (1H, d, J=9 Hz), 6.88-6.94 (2H, m), 7.07-7.14 (2H, m), 7.30 (1H, d, J=3 Hz), 7.92 (1H, t, J=2 Hz), 8.40 (1H, d, 2 Hz), 9.06 (1H, d, J=2 Hz).

LC/MS t=4.14 min [MH+] 499/501/503.

### b) 5-{2-[5-chloro-2-(2-chloro-4-fluoro-benzyloxy)-phenyl]-5-methyl-pyrrol-1-yl}-nicotinic acid

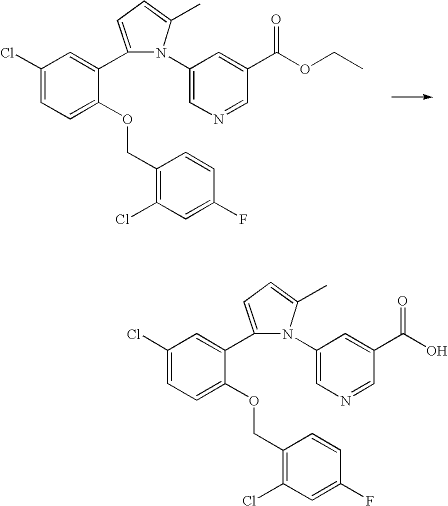

5-{2-[5-Chloro-2-(2-chloro-4-fluoro-benzyloxy)-phenyl]-5-methyl-pyrrol-1-yl}-nicotinic acid ethyl ester (39 mg, 0.08 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (28 mg, 76%).

¹H NMR (400 MHz, d6-DMSO) 2.10 (3H, s), 4.78 (2H, s), 6.13 (1H, dd, J=1, 3.5 Hz), 6.29 (1H, d, J=3.5 Hz), 6.93 (1H, d, J=9 Hz), 7.07-7.14 (1H, m), 7.19 (1H, ddd, J=2.5, 9 Hz), 7.23-7.29 (2H, m), 7.46 (1H, dd, J=2.5, 9 Hz), 7.78 (1H, t, J=2 Hz), 8.40 (1H, d, J=2 Hz), 8.94 (1H, d, J=2 Hz), 13.47 (1H, broad s).

LC/MS t=4.08 min [MH−] 469/471/473.

### Example 36

### 5-{2-[5-Chloro-2-(3,4-dichloro-benzyloxy)-phenyl]-5-methyl-pyrrol-1-yl}-nicotinic acid

### a) 5-{2-[5-Chloro-2-(3,4-dichloro-benzyloxy)-phenyl]-5-methyl-pyrrol-1-yl}-nicotinic acid ethyl ester

3,4-Dichloro-benzyl bromide (0.036 ml, 0.21 mmol) was added to 5-{2-[5-Chloro-2-(hydroxy)-phenyl]-5-methyl-pyrrol-1-yl}nicotinic acid ethyl ester (50 mg, 0.14 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO4), filtered and concentrated. The residue was purified by chromatography on silica gel with isohexane/EtOAc (15%) as eluant, to give the title compound (31 mg, 43%).

¹H NMR (400 MHz, CDCl₃) 1.34 (3H, t, J=7 Hz), 2.18 (3H, s), 4.33 (2H, q, J=7 Hz), 4.64 (2H, s), 6.18 (1H, dd, J=1, 3.5 Hz), 6.31 (1H, d, J=3.5 Hz), 6.54 (1H, d, J=9 Hz), 6.88 (1H, dd, J=2, 8 Hz), 7.09-7.13 (2H, m), 7.32 (1H, d, J=3 Hz), 7.38 (1H, d, J=8.5 Hz), 7.90 (1H, t, J=2 Hz), 8.39 (1H, d, 2 Hz), 9.05 (1H, d, J=2 Hz).

LC/MS t=4.23 min [MH+] 515/517/519.

### b) 5-{2-[5-Chloro-2-(3,4dichloro-benzyloxy)-phenyl]-5-methyl-pyrrol-1-yl}-nicotinic acid

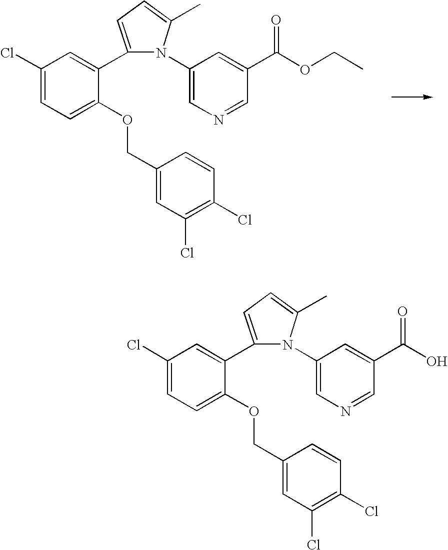

5-{2-[5-Chloro-2-(3,4-dichloro-benzyloxy)-phenyl]-5-methyl-pyrrol-1-yl}nicotinic acid ethyl ester (31 mg, 0.06 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (23 mg, 79%).

¹H NMR (400 MHz, d6-DMSO) 2.13 (3H, s), 4.80 (2H, s), 6.16 (1H, dd, J=1, 3.5 Hz), 6.30 (1H, d, J=3.5 Hz), 6.83 (1H, d, J=9 Hz), 7.05 (1H, dd, J=2, 8.5 Hz), 7.23-7.31 (3H, m), 7.58 (1H, d, J=8.5 Hz), 7.84 (1H, t, J=2 Hz), 8.44 (1H, d, J=2 Hz), 8.94 (1H, d, J=2 Hz), 13.50 (1H, broad s).

LC/MS t=4.26 min [MH+] 487/489/491.

### Example 37

### 5-{2-[5-Chloro-2-(2,4-difluoro-benzyloxy)-phenyl]-5-methyl-pyrrol-1-yl}-nicotinic acid

### a) 5-{2-[5-Chloro-2-(2,4-difluoro-benzyloxy)-phenyl]-methyl-pyrrol-1-yl}-nicotinic acid ethyl ester

2,4-Difluoro-benzyl bromide (0.027 ml, 0.21 mmol) was added to 5-{2-[5-Chloro-2-(hydroxy)-phenyl]-5-methyl-pyrrol-1-yl}nicotinic acid ethyl ester (50 mg, 0.14 mmol) and K₂CO₃ (43 mg, 0.31 mmol) in DMF (1 ml) and the reaction mixture was heated at 60° C. for 16 hours. Upon cooling the reaction mixture was diluted with EtOAc, washed with water, brine, dried (MgSO4), filtered and concentrated. The residue was purified by chromatography on silica gel with isohexane/EtOAc (15%) as eluant, to give the title compound (40 mg, 59%).

¹H NMR (400 MHz, CDCl₃) 1.36 (3H, t, J=7 Hz), 2.16 (3H, s), 4.35 (2H, q, J=7 Hz), 4.71 (2H, s), 6.16 (1H, dd, J=1, 3.5 Hz), 6.29 (1H, d, J=3.5 Hz), 6.62 (1H, d, J=9 Hz), 6.75-6.84 (2H, m), 6.97 (1H, ddd, J=2, 7 Hz), 7.12 (1H, dd, J=2, 9 Hz), 7.28 (2H, d, J=2 Hz), 7.90 (1H, t, J=2 Hz), 8.37 (1H, d, 2 Hz), 9.05 (1H, d, J=2 Hz).

LC/MS t=4.00 min [MH+] 482/484.

### b) 5-{2-[5-Chloro-2-(2,4-difluoro-benzyloxy)-phenyl]-5-methyl-pyrrol-1-yl}-nicotinic acid

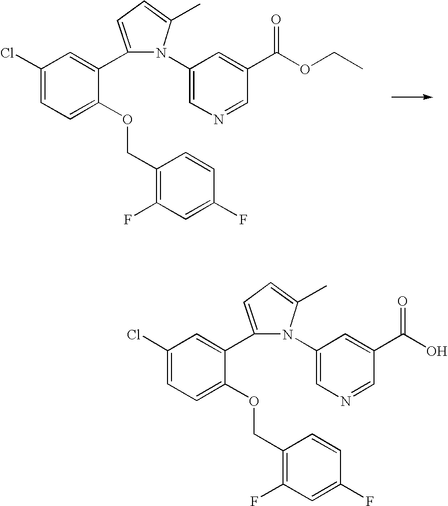

5-{2-[5-Chloro-2-(2,4-difluoro-benzyloxy)-phenyl]-5-methyl-pyrrol-1-yl}-nicotinic acid ethyl ester (40 mg, 0.08 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (30 mg, 80%).

¹H NMR (400 MHz, d6-DMSO) 2.08 (3H, s), 4.77 (2H, s), 6.12 (1H, broad d, J=3.5 Hz), 6.26 (1H, d, J=3.5 Hz), 6.97 (1H, d, J=9 Hz), 7.05 (1H, ddd, J=2.5, 9 Hz), 7.11-7.29 (4H, m), 7.76 (1H, t, J=2 Hz), 8.36 (1H, d, J=2 Hz), 8.93 (1H, d, J=2 Hz), 13.47 (1H, broad s).

LC/MS t=3.88 min [MH−] 453/455.

### Example 38

### 5-{2-[5-Bromo-2-(4-methoxy-benzyloxy)-phenyl]-5-methyl-pyrrol-1-yl}-nicotinic acid

### a) 5-{2-[5-Bromo-2-(4-methoxy-benzyloxy)-phenyl]-5-methyl-pyrrol-1-yl}-nicotinic acid ethyl ester

1-[5-Bromo-2-(4-methoxy-benzyloxy)-phenyl]-pentane-1,4-dione (134 mg, 0.34 mmol) and 5-amino-nicotinic acid ethyl ester (62 mg, 0.37 mmol) were heated in toluene (0.5 ml) in a sealed vessel at 150° C. for 3 days. Upon cooling, the mixture was diluted with EtOAc and washed with 2M HCl and saturated NaHCO₃, dried (MgSO₄), filtered and concentrated. The residue was purified by chromatography, using Biotage^{□}, with isohexane containing a gradient of EtOAc (15-20%) as eluant, to give the title compound (60 mg, 36%).

LC/MS t=3.99 min [MH+] 521/523

### b) 5-{2-[5-Bromo-2-(4-methoxy-benzyloxy)-phenyl]-5-methyl-pyrrol-1-yl}-nicotinic acid

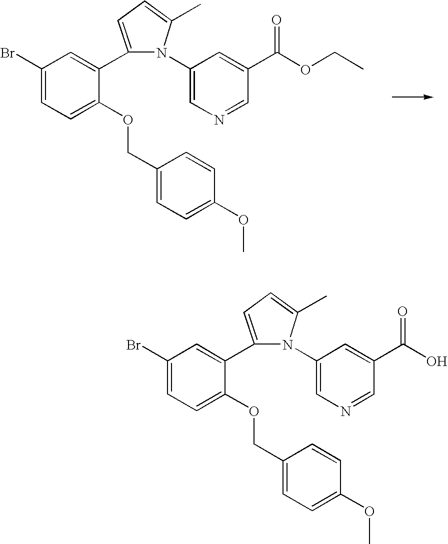

5-{2-[5-Bromo-2-(4-methoxy-benzyloxy)-phenyl]-5-methyl-pyrrol-1-yl}-nicotinic acid ethyl ester (1 0 mg, 0.02 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (7 mg, 74%).

¹H NMR (400 MHz, CDCl₃) 2.15 (3H, s), 3.76 (3H, s), 4.60 (2H, s), 6.16 (1H, d, J=3.5 Hz), 6.30 (1H, d, J=3.5 Hz), 6.56 (1H, d, J=9 Hz), 6.82 (2H, d, J=9 Hz), 6.98 (2H, d, J=9 Hz), 7.23 (1H, dd, J=2.5, 9 Hz), 7.46 (1H, d, J=2.5 Hz), 7.91 (1H, t, J=2 Hz), 8.36 (1H, d, J=2 Hz), 9.09 (1H, d, J=2 Hz).

LC/MS t=9.83 min [MH+] 493/49.

### Example 39

### 3-{2-[5-Chloro-2-(benzyloxy)-phenyl]-5-methylpyrrol-1-yl}-6-chlorobenzoic acid

### a) 3-{2-[5-Chloro-2-(benzyloxy)-phenyl]-5-methylpyrrol-1-yl}-6-chloro-benzoic acid methyl ester

1-[5-Chloro-2-(benzyloxy)-phenyl]-pentane-1,4-dione (160 mg, 0.5 mmol) was treated with 5-amino-2-chloro-benzoic acid methyl ester (100 mg, 0.55 mol) (Brown et al., WO0055120), and p-toluenesulfonic acid (˜30 mg) in toluene (4 ml). The reaction mixture was then refluxed over 18 hrs under nitrogen, evaporated down to an oil, dissolved in as little DCM as possible, and placed on a Water's silica cartridge (10g) saturated with iso-hexane. The column was then eluted with iso-hexane (˜50 ml) followed by an Et₂O/iso-hexane gradient mixture starting at 10% Et₂O to give the title compound (34 mg, 7%).

¹H NMR (400 MHz, CDCl₃) 2.23 (3H, s), 3.8 (3H, s), 4.72 (2H, s), 6.12 (1H, d, J=3 Hz), 6.28 (1H, d, J=3 Hz), 6.58 (1H, d, J=9 Hz), 6.94 (1H, dd, J=2, 8 Hz), 7.69-7.05 (2H, m), 7.08 (1H, dd, J=2.8 Hz), 7.22 (5H, m), 7.48 (1H, d, J=2 Hz).

LC/MS t=4.14 min, [MH+] 466/468/470.

### b) 3-{2-[5-Chloro-2-(benzyloxy)-phenyl]-5-methylpyrrol-1-yl}-6-chlorobenzoic acid

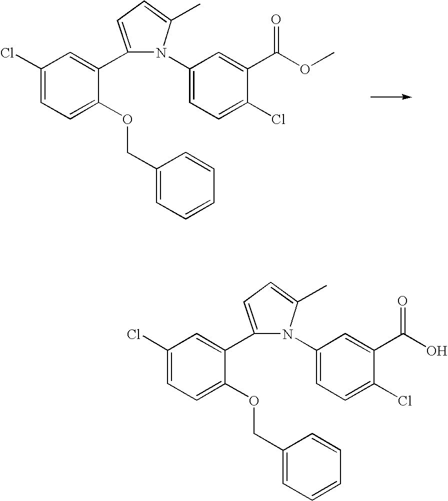

3-[2-(5-Chloro-2-benzyloxy-phenyl)-5-methylpyrrol-1-yl]-6-chlorobenzoic acid methylester (34 mg) was treated with 2M NaOH (3 ml) in MeOH (4 ml) and heated at reflux for 2 hrs under nitrogen. The reaction mixture was then reduced in vacuo, diluted with water (˜10 ml), treated with 2M HCl (˜3 ml), then adjusted to pH˜4 with a few drops of glacial acetic acid. The aqueous was extracted with DCM (2×10 ml). The organic layer was then dried with MgSO₄, filtered and evaporated to give the title compound (24 mg, 73%).

¹H NMR 400 MHz, CDCl₃) 2.15 (3H, s), 4.72 (2H, s), 6.13 (1H, d, J=2 Hz), 6.29 (1H, d, J=3 Hz), 6.59 (1H, d, J=8 Hz), 6.96-7.06 (3H, m), 7.09 (1H, dd, J=2,8 Hz), 7.22-7.33 (5H,m), 7.64 (1H,d, J=2 Hz).

LC/MS t=4.26 min, [MH+] 452/454/456.

### Example 40

### 3-{2-[5-Chloro-2-(benzyloxy)-phenyl]-5-methylpyrrol-1-yl}-5-bromobenzoic acid

### a) 3-{2-[5-Chloro-2-(benzyloxy)-phenyl]-5-methylpyrrol-1-yl}-5-bromobenzoic acid methyl ester

Procedure as for 3-{2-[5-Chloro-2-(benzyloxy)-phenyl]-5-methylpyrrol-1-yl}-6-chloro-benzoic acid methyl ester, using 3-amino-5-bromo-benzoic acid methyl ester (ex. SALOR) to give the title compound (35 mg, 38%).

¹H NMR (400 MHz, CDCl₃) 2.13 (3H, s), 3.82 (3H, s), 4.74 (2H, s), 6.12 (1H, d, J=3 Hz), 6.28 (1H, d, J=3 Hz), 6.60 (1H, d, J=9 Hz), 7.04-7.11 (3H, m), 7.24-7.32 (5H, m), 7.60 (1H, s), 8.02 (1H, s).

LC/MS t=4.26 min [MH+] 510/512/514.

### b) 3-{2-[5-Chloro-2-(benzyloxy)-phenyl]-5-methylpyrrol-1-yl}-5-bromobenzoic acid

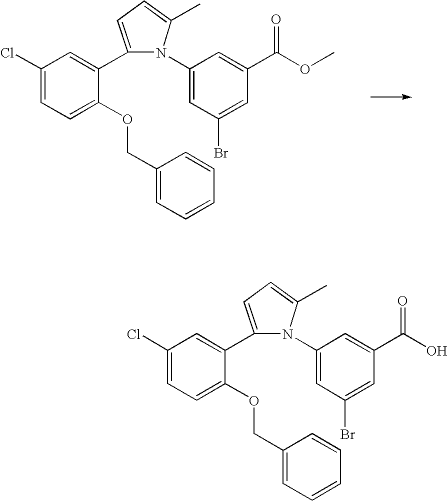

Procedure as for 3-{2-[5-chloro-2-(benzyloxy)-phenyl]-5-methylpyrrol-1-yl}-6-chlorobenzoic acid, to give title compound (34 mg, 100%).

¹H NMR (400 MHz, CDCl₃) 2.16 (3H, s), 4.74 (2H, s), 6.13 (1H, d, J=3 Hz), 6.3 (1H, d, J=3 Hz), 6.61 (1H, d, J=9 Hz), 7.4-7.12(3H, m), 7.23-7.35 (5H,m), 7.60, (1H, s), 8.06 (1H, s).

LC/MS t=4.30 min [MH+] 496/498/500.

### Example 41

### 3-{2-[5-Chloro-2-(4-fluorobenzyloxy)-phenyl]-5-methylpyrrol-1-yl}-4-acetylamino-benzoic acid

### a) 5-Chloro-2-(4-fluoro-benzyloxy)-benzaldehyde

Procedure as for 2-benzyloxy-5-chloro-benzaldehyde to give the title compound.

LCMS t=3.56 min[MNH₄⁺] 282

### b) 1-[5-Chloro-2-(4-fluoro-benzyloxy)-phenyl]-pentane-1,4-dione

Procedure as for 1-(2-benzyloxy-5-chloro-phenyl)-pentane-1,4dione to give the title compound.

LCMS rt=3.46

### c) 3-{2-[5-Chloro-2-(4-fluorobenzyloxy)-phenyl]-5-methylpyrrol-1-yl}-5-acetylamino-benzoic acid

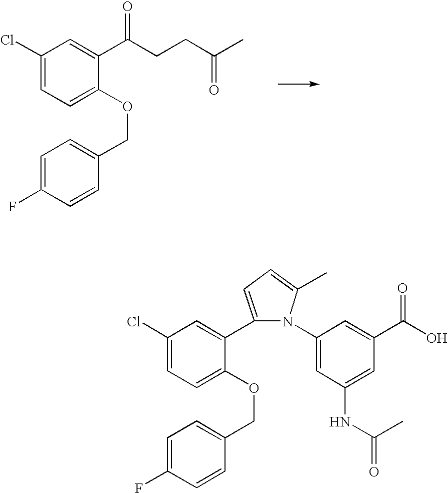

Procedure as for 3-{2-[5-chloro-2-(benzyloxy)-phenyl]-5-methylpyrrol-1-yl}-6-chloro-benzoic acid methyl ester using the appropriate amine (Hakansson et al, U.S. Pat. No. 3,907,880), to give the title compound (35 mg, 14%).

¹H NMR (400 MHz, CDCl₃) 2.14 (3H, s), 2.16 (3H, s), 4.71 (2H, s), 5.98 (1H, d, J=3 Hz), 6.12 (1H,d, J=3 Hz), 6.57 (1H, d, J=8 Hz), 6.93-7.10 (5H, m), 7.18-7.25 (2H, m), 7.44 (1H, s), 7.48 (1H, s).

LC/MS t=3.66 min, [MH+] 493/495.

### Example 42

### 3-{2-[5-Chloro-2-(4-fluorobenzyloxy)-phenyl]-5-methylpyrrol-1-yl}-5-trifluoromethyl benzoic acid

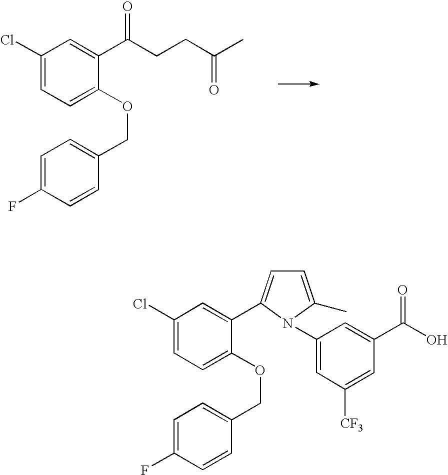

Procedure as for 3-{2-[5-chloro-2-(benzyloxy)-phenyl]-5-methylpyrrol-1-yl}-6-chloro-benzoic acid methyl ester using the appropriate amine, to give the title compound (70 mg, 28%).

¹H NMR (400 MHz, CDCl₃) 2.17 (3H, s), 4.64 (2H, s), 6.16 (1H, d, J=3 Hz), 6.31 (1H, d, J=3 Hz), 6.58 (1H, d, J=3 Hz), 6.92-7.06 (4H, m), 7.12 (1H, dd, J=2,8 Hz), 7.3 (1H, d, J=2.4 Hz), 7.40 (1H,s), 7.85 (1H, s), 8.17 (1H, s).

LC/MS t=4.25 min [MH+] 504/506.

### Example 43

### 3-{2-[5-Chloro-2-(4-fluorobenzyloxy)-phenyl]-5-methylpyrrol-1-yl}naphthalene-1-carboxylic acid

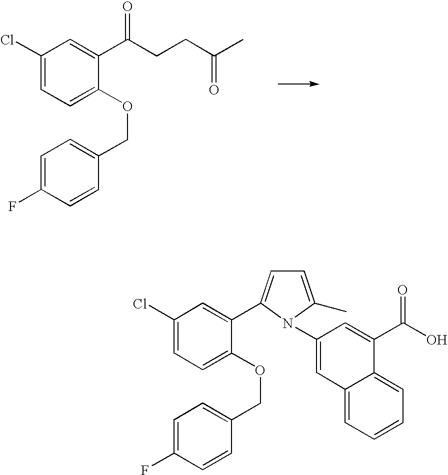

Procedure as for 3-{2-[5-chloro-2-(benzyloxy)-phenyl]-5-methylpyrrol-1-yl}-6-chloro-benzoic acid methyl ester using the appropriate amine (Ruminski et al., WO9708145) to give the title compound (70 mg, 30%).

¹H NMR (400 MHz, CDCl₃) 2.20 (3H, s), 4.60 (2H, s), 6.18 (1H, d, J=3 Hz), 6.33 (1H, d, J=3 Hz), 6.46 (1H, d, J=8 Hz), 6.82-6.92 (4H, m), 7.03 (1H, dd, J=2.5, 9 Hz), 7.39 (1H, d, J=2.5 Hz), 7.49-7.56 (1H, m), 7.58 (1H, d, J=2 Hz), 7.62-7.68 (2H, m), 8.06 (1H, d, J=2 Hz), 9.02 (1H, d, J=9 Hz).

LC/MS t=4.15 min [MH+] 486/488.

### Example 44

### 3-{2-[5-Chloro-2-(4-fluoro-benzyloxy)-phenyl]-5-methyl-pyrrol-1-yl}-6-fluoro-benzoic acid

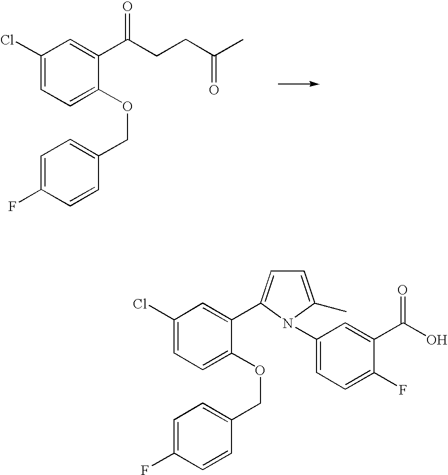

Procedure as for 3-{2-[5-chloro-2-(benzyloxy)-phenyl]-5-methylpyrrol-1-yl}-6-chloro-benzoic acid methyl ester using the appropriate amine, to give the title compound (80 mg, 35%).

¹H NMR (400 MHz, CDCl₃) 2.13 (3H, s), 4.70 (2H, s),6.12 (1H, d, J=3 Hz), 6.28 (1H, d, J=3 Hz), 6.60 (1H, d, J=9 Hz), 6.93-7.02 (3H, m), 7.02-7.12 (5H, m), 7.64 (1H, dd, J=2.5, 7 Hz).

LC/MS t=4.02 min [MH+] 454/456.

### Example 45

### 3-{2-[5-Chloro-2-(4-fluorobenzyloxy)-phenyl]-5-methylpyrrole-1-yl}-fluorobenzoic acid

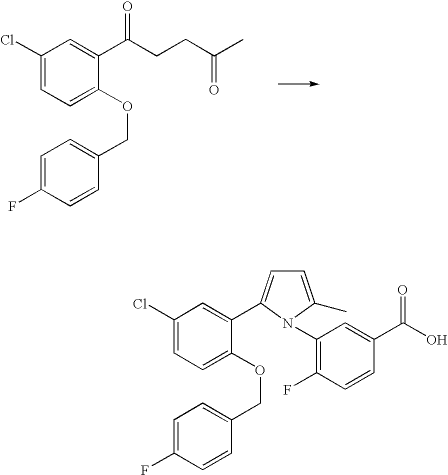

Procedure as for 3-{2-[5-chloro-2-(benzyloxy)-phenyl]-5-methylpyrrol-1-yl}-6-chloro-benzoic acid methyl ester using the appropriate amine, to give the title compound (110 mg, 48%).

LC/MS t=3.96 min [MH+] 454.

### Example 46

### 3-{2-[4-Chloro-2-(4-fluorobenzyloxy)-phenyl]-5-methylpyrrol-1-yl}-6-methyl-benzoic acid

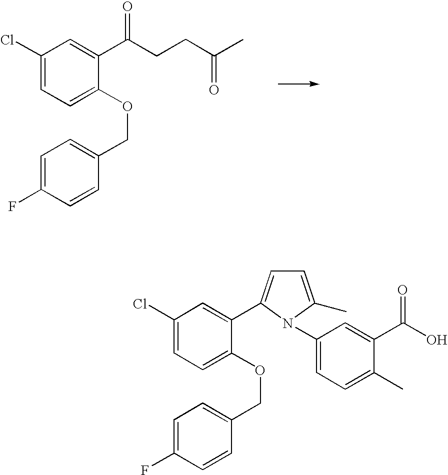

Procedure as for 3-{2-[5-chloro-2-(benzyloxy)-phenyl]-5-methylpyrrol-1-yl}-6-chloro-benzoic acid methyl ester using the appropriate amine (Ashton et al, J. Med. Chem., 1996, 39(17), 3343-3356, to give the title compound (30 mg, 26%).

¹H NMR (400 MHz, CDCl₃) 2.14 (3H, s), 2.62 (3H, s), 4.71 (2H, s), 6.12 (1H, d, J=3 Hz), 6.28 (1H, d, J=3 Hz), 6.57 (1H, d, J=8 Hz), 6.92-7.12 (7H, m), 7.24 (1H, d, J=2 Hz), 7.72 (1H, d, J=2 Hz).

LC/MS t=4.04 min [MH+] 450/452.

### Example 47

### 3-{2-[5-Chloro-2-(4-fluoro-benzyloxy)-phenyl]-5-methyl-pyrrol-1-yl}-6-chloro-benzoic acid

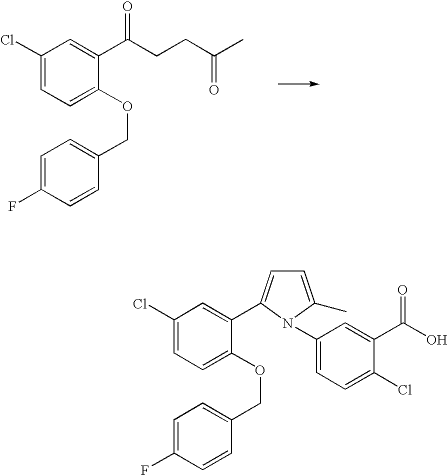

Procedure as for 3-{2-[5-chloro-2-(benzyloxy)-phenyl]-5-methylpyrrol-1-yl}-6-chloro-benzoic acid methyl ester using the appropriate amine, to give the title compound (50 mg, 21%).

¹H NMR (400 MHz, CDCl₃) 2.15 (3H, s), 4.68 (2H, s), 6.13 (1H, d, J=3 Hz), 6.27 (1H, d, J=3 Hz), 6.58 (1H, d, J=8 Hz), 6.95-7.05 (5H, m), 7.10 (1H, dd, J=2, 8 Hz), 7.24-7.32 (2H, m), 7.62 (1H, d, J=2 Hz).

LC/MS t=4.24 min [MH+] 470/472/474.

### Example 48

### 3-{2-[5-Chloro-2-(4-fluorobenzyloxy)-phenyl]-5-methylpyrrol-1-yl}-2,5,6-trifluorobenzoic acid

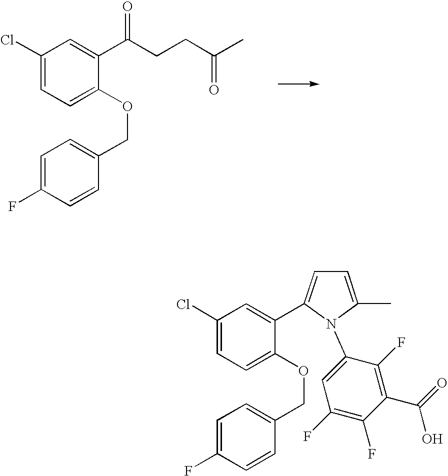

Procedure as for 3-{2-[5-chloro-2-(benzyloxy)-phenyl]-5-methylpyrrol-1-yl}-6-chloro-benzoic acid methyl ester using the appropriate amine, to give the title compound (20 mg, 8%).

¹H NMR (400 MHz, CDCl₃) 2.10 (3H, s), 4.78 (2H, s), 6.13 (1H, d, J=3 Hz), 6.27 (1H, d, J=3 Hz), 6.64 (1H, d, J=8 Hz), 6.86-7.03 (4H, m), 7.03-7.15 (3H, m).

LC/MS t=4.59 min [MH+] 490/492.

### Example 49

### 3-{2-[5-Chloro-2-(2,4-difluorobenzyloxy)-phenyl]-5-methylpyrrol-1-yl}-6-methyl-benzoic acid

### a) 5-Chloro-2-(2,4-difluoro-benzyloxy)-benzaldehyde

Procedure as for 2-benzyloxy-5-chloro-benzaldehyde to give the title compound.

LCMS t=3.60 min.

### b) 1-[5-Chloro-2-(2,4-difluoro-benzyloxy)-phenyl]-pentane-1,4-dione

Procedure as for 1-[5-chloro-2-(benzyloxy)-phenyl]-pentane-1,4-dione to give the title compound.

LCMS t=3.49 min [MNa⁺] 375

### c) 3-{2-[5-Chloro-2-(2,4-difluorobenzyloxy)-phenyl]-5-methylpyrrol-1-yl}-6-methyl-benzoic acid

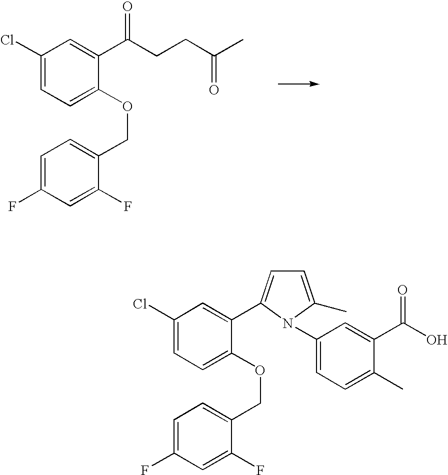

Procedure as for 3-{2-[5-chloro-2-(benzyloxy)-phenyl]-5-methylpyrrol-1-yl}-6-chloro-benzoic acid methyl ester using the appropriate amine, to give the title compound (45 mg, 20%).

¹H NMR (400 MHz, CDCl₃) 2.15 (3H, s), 2.62 (3H, s), 4.76 (2H, s), 6.12 (1H, d, J=3 Hz), 6.28 (1H, d, J=3 Hz), 6.61 (1H,d J=9 Hz), 6.72-6.82 (2H, m), 6.94-7.0 (2H, m), 7.05-7.14 (2H,m), 7.20 (1H, d, J=2.4 Hz), 7.74 (1H, d, J=2 Hz).

LC/MS t=4.07 min [MH+] 468/470.

### Example 50

### 3-{2-[5-Chloro-2-(2,4-difluorobenzyloxy)-phenyl]-5-methylpyrrol-1-yl}-6-fluoro-benzoic acid

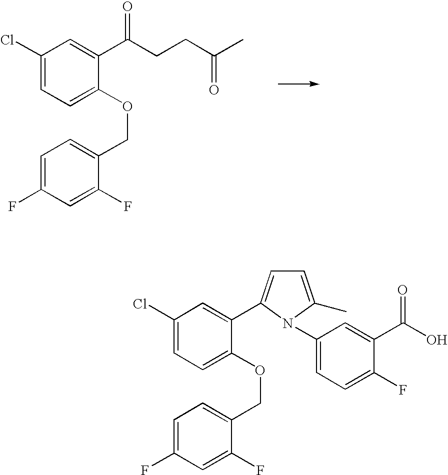

Procedure as for 3-{2-[5-chloro-2-(benzyloxy)-phenyl]-5-methylpyrrol-1-yl}-6-chloro-benzoic acid methyl ester using the appropriate amine, to give the title compound (30 mg, 13%).

¹H NMR (400 MHz, CDCl₃) 2.13 (3H, s), 4.76 (2H, s), 6.12 (1H, d, J=3 Hz), 6.26 (1H, d, J=3 Hz), 6.65 (1H, d, J=9 Hz), 6.74-6.87 (2H, m) 6.97-7.07 (2H, m), 7.07-7.15 (2H, m), 7.22 (1H, d, J=2 Hz), 7.68 (1H, dd, J=2, 7 Hz).

LC/MS t=07 min [MH+] 472.

### Example 51

### 3-{2-[5-Chloro-2-(2,4-difluorobenzyloxy)-phenyl]-5-methylpyrrol-1-yl}-naphthalene-1-carboxylic acid

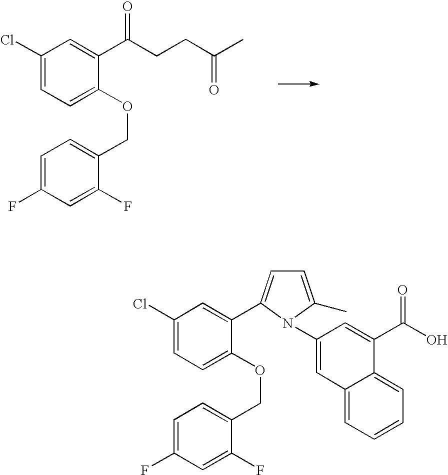

Procedure as for 3-{2-[5-chloro-2-(benzyloxy)-phenyl]-5-methylpyrrol-1-yl}-6-chloro-benzoic acid methyl ester using the appropriate amine, to give the title compound (60 mg, 24%).

¹H NMR (400 MHz, CDCl₃) 2.20, (3H, s), 4.69 (2H, s), 6.18 (1H, d, J=3 Hz), 6.32 (1H, d, J=3 Hz), 6.51 (1H, d, J=8 Hz), 6.58-6.65 (1H, m), 6.69-6.82 (2H, m), 7.05 (1H, dd, J=2,4,8 Hz), 7.36 (1H, d, J=2.4 Hz), 7.50-7.56 (1H, m), 7.60-7.70 (3H, m), 8.10 (1H, d, J=2 Hz), 9.03(1H, d, J=8 Hz).

LC/MS t=4.22 min [MH+] 504/506.

### Example 52

### 3-{2-[5-Chloro-2-(2,4-difluorobenzyloxy)-phenyl]-5-methylpyrrol-1-yl}-5-trifluoromethylbenzoic acid

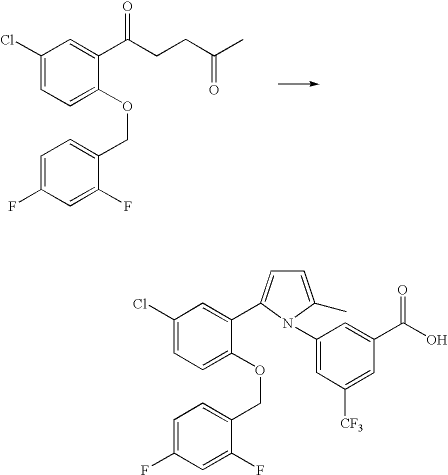

Procedure as for 3-{2-[5-chloro-2-(benzyloxy)-phenyl]-5-methylpyrrol-1-yl}-6-chloro-benzoic acid methyl ester using the appropriate amine, to give the title compound (18 mg, 7%).

¹H NMR (400 MHz, CDCl₃) 2.17 (3H, s), 4.70 (2H, s), 6.16 (1H, d, J=3 Hz), 6.30 (1H, d, J=3 Hz), 6.62 (1H, d, J=8 Hz), 6.73-6.84 (2H, m), 6.92-7.01 (1H, m), 7.13 (1H, dd, J=2.4, 8 Hz), 7.28 (1H, d, J=2 Hz), 7.42 (1H, s), 7 86 (1H, s), 7.95 (1H, s).

LC/MS t=5.28 min [MH+] 522/524.

### Example 53

### 3-{2-[5-Chloro-2-(2,4-difluorobenzyloxy)phenyl]-5-methylpyrrol-1-yl}-4-fluorobenzoic acid

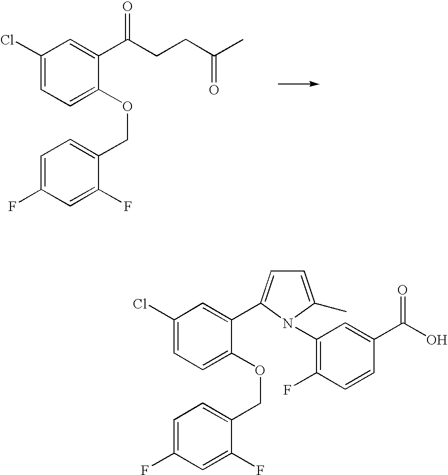

Procedure as for 3-{2-[5-chloro-2-(benzyloxy)-phenyl]-5-methylpyrrol-1-yl}-6-chloro-benzoic acid methyl ester using the appropriate amine, to give the title compound (60 mg, 25%).

¹H NMR (400 MHz, CDCl₃) 2.10 (3H, s), 4.82 (2H, d, J=2 Hz), 6.1 (1H, d J=3 Hz), 6.31 (1H, d, J=3 Hz), 6.61 (1H, d, J=8 Hz), 6.74-6.83 (2H, m), 7.00-7.09 (2H, m), 7.12 (1H, t, J=7 Hz), 7.2 (1H, d, J=2 Hz), 7.77 (1H, dd, J=2, 7 Hz), 7.98-8.45 (1H, m).

LC/MS t=4.01 min [MH+] 472/474.

### Example 54

### 3-{2-[5-Chloro-2-(2,4-difluorobenzyloxy)phenyl]-5-methylpyrrol-1-yl}-5-acetylamino-benzoic acid

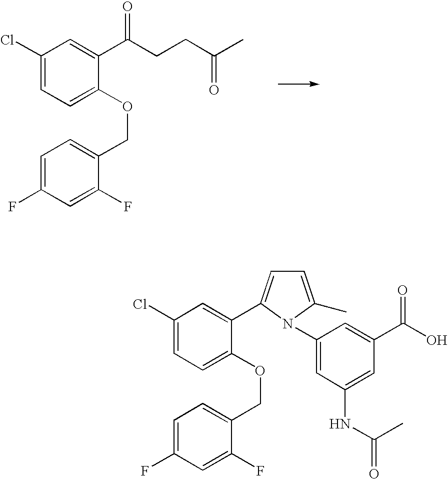

Procedure as for 3-{2-[5-chloro-2-(benzyloxy)-phenyl]-5-methylpyrrol-1-yl}-6-chloro-benzoic acid methyl ester using the appropriate amine, to give the title compound (60 mg, 23%).

¹H NMR (400 MHz, CDCl₃) 2.26 (3H,s), 2.15 (3H, s), 2.16 (3H,s) 4.78 (2H, s), 6.11 (1H, d, J=3 Hz), 6.28 (1H, d, J=3 Hz), 6.62 (1H, d, J=8 Hz), 6.72-6.84 (2H, m), 7.00-7.08 (2H, m), 7.20 (1H, d, J=2 Hz), 7.46 (1H, s), 7.60 (1H, s), 7.95 (1H, s).

LC/MS t=3.71 min [MH+] 511/513.

### Example 55

### 3-{2-[5-Chloro-2-(2,4-difluorobenzyloxy)phenyl]-5-methylpyrrol-1-yl}-6-chloro-benzoic acid

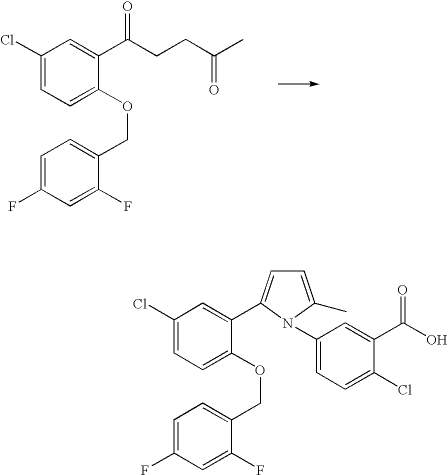

Procedure as for 3-{2-[5-chloro-2-(benzyloxy)-phenyl]-5-methylpyrrol-1-yl}-6-chloro-benzoic acid methyl ester using the appropriate amine, to give the title compound (45 mg, 18%).

¹H NMR (400 MHz, CDCl₃) 2.16 (3H, s), 4.76 (2H, s), 6.13 (1H, d, J=3 Hz), 6.27 (1H, d, J=3 Hz), 6.64 (1H, d, J=9 Hz), 6.75-6.85 (2H, m), 6.90-6.98 (2H, m), 7.02 (1H, dd, J=2, 8 Hz), 7.12 (1H, dd, J=2, 8 Hz), 7.30 (1H, d, J=8 Hz), 7.65 (1H, d, J=2 Hz).

LC/MS t=4.25 min [MH+] 488/490/492.

### Example 56

### 3-{2-[5-Chloro-2-(2,4-difluorobenzyloxy)-phenyl]-5-methylpyrrol-1-yl}-2,5,6-trifluorobenzoic acid

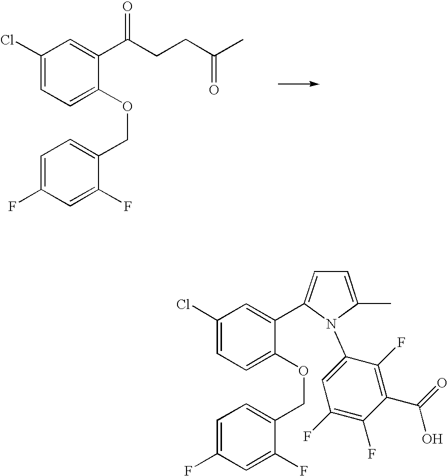

Procedure as for 3-{2-[5-chloro-2-(benzyloxy)-phenyl]-5-methylpyrrol-1-yl}-6-chloro-benzoic acid methyl ester using the appropriate amine, to give the title compound (27 mg, 10%).

¹H NMR (400 MHz, CDCl₃) 2.10 (3H, s), 4.84 (2H, dd, J=7, 20 Hz), 6.30 (1H, d, J=3 Hz), 6.26 (1H, d, J=3 Hz), 6.68 (1H, d, J=8 Hz), 6.74-6.82 (2H, m), 6.89-6.99 (1H, m), 6.99-7.08 (1H, m), 7.14 (1H, dd, J=2, 9 Hz), 7.25 (1H, d, 2 Hz).

LC/MS t=4.64 min [MH+] 508/510.

### Example 57

### 3-{2-[5-Bromo-2-(2,4-difluorobenzyloxy)phenyl]-5-methylpyrrol-1-yl}-6-chlorobenzoic acid

### a) 5-Bromo-2-(2,4-difluoro-benzyloxy)-benzaldehyde

Procedure as for 2-benzyloxy-5-chloro-benzaldehyde to give the title compound.

LCMS t=3.71 min [MNa⁺] 349/351.

### b) 1-[5-Bromo-2-(2,4-difluoro-benzyloxy)-phenyl]-pentane-1,4-dione

Procedure as for 1-[5-chloro-2-(benzyloxy)-phenyl]-pentane-1,4-dione to give the title compound.

LCMS t=3.41 min [MH⁺] 397/399.

### c) 3-{2-[5-Bromo-2-(2,4-difluorobenzyloxy)phenyl]-5-methylpyrrol-1-yl}-6-chlorobenzoic acid

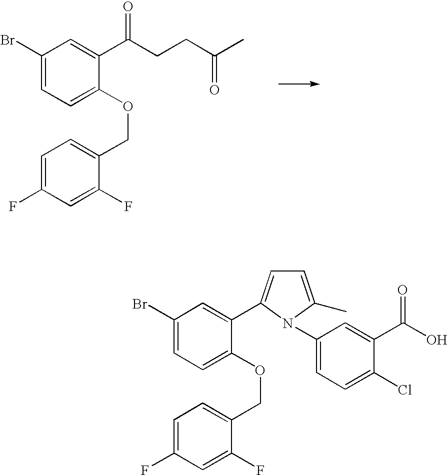

Procedure as for 3-{2-[5-chloro-2-(benzyloxy)-phenyl]-5-methylpyrrol-1-yl}-6-chloro-benzoic acid methyl ester using the appropriate amine, to give the title compound (80 mg, 30%).

¹H NMR (400 MHz, CDCl₃) 2.15 (3H, s), 4.75 (2H, s), 6.12 (1H, d, J=3 Hz), 6.26 (1H, d, J=3 Hz), 6.58 (1H, d, J=8 Hz), 6.74-6.85 (2H, m), 6.89-6.97 (1H,m), 7.01 (1H, dd, J=2, 8 Hz), 7.23-7.29 (1H, m), 7.32 (1H, d, J=8 Hz), 7.42 (1H, d, J=2 Hz), 7.66 (1H, d, J=2 Hz).

LC/MS t=4.25 min [MH+] 532/534/536.

### Example 58

### 3-{2-[5-Bromo-2-(2,4-difluorobenzyloxy)-phenyl]-5-methylpyrrol-1-yl}-4-chlorobenzoic acid

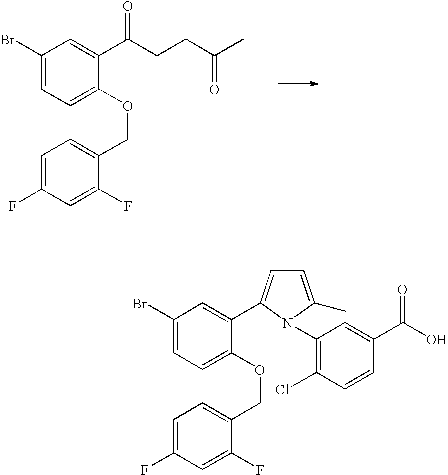

Procedure as for 3-{2-[5-chloro-2-(benzyloxy)-phenyl]-5-methylpyrrol-1-yl}-6-chloro-benzoic acid methyl ester using the appropriate amine, to give the title compound (125 mg, 47%).

LC/MS t=4.24 min [MH+] 532/534/536.

### Example 59

### 3-{2-[5-Bromo-2-(2,4-difluorobenzyloxy)-phenyl]-5-methylpyrrol-1-yl}-naphthalene-1-carboxylic acid

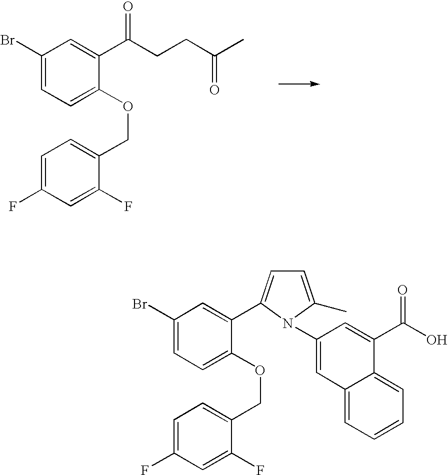

Procedure as for 3-{2-[5-chloro-2-(benzyloxy)-phenyl]-5-methylpyrrol-1-yl}-6-chloro-benzoic acid methyl ester using the appropriate amine, with 4 Å molecular sieve (˜0.5g) to give the title compound (25 mg, 91%).

¹H NMR (400 MHz, CDCl₃) 2.20 (3H, s), 4.68 (2H, s), 6.18 (1H, d, J=3 Hz), 6.32 (1H, d, J=3 Hz), 6.45 (1H, d, J=9 Hz), 6.56-6.64 (1H, m), 6.68-6.79 (2H, m), 7.19 (1H, dd, J=2, 8 Hz), 7.49-7.58 (2H, m), 7.58-7.72 (3H, m), 8.09 (1H, d, J=2 Hz), 9.03 (1H, d, J=8 Hz).

LC/MS t=4.26 min [MH+] 548/550.

### Example 60

### 3-{2-[5-Bromo-2-(2,4-difluorobenzyloxy)-phenyl]-5-methylpyrrol-1-yl}-5-acetylamino-benzoic acid

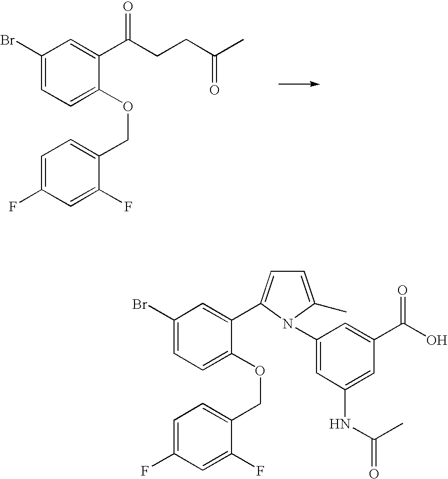

Procedure as for 3-{2-[5-chloro-2-(benzyloxy)-phenyl]-5-methylpyrrol-1-yl}-6-chloro-benzoic acid methyl ester using the appropriate amine, with 4 Å molecular sieves (˜0.5g) to give the title compound (25 mg, 90%).

¹H NMR (400 MHz, CDCl₃) 2.16 (3H, s),2.16 (6H, s), 4.77 (2H, s), 6.11 (1H, d, J=3 Hz), 6.28 (1H, d, J=3 Hz), 6.56 (1H, d, J=8 Hz), 6.71-6.84 (2H, m), 6.98-7.06 (1H, m), 7.18 (1H, dd, J=2, 9 Hz), 7.35 (2H, d, J=2 Hz), 7.45 (1H, s), 7.58 (1H, s), 7.97 (1H, s).

LC/MS t=3.76 min [MH+] 555/557.

### Example 61

### 3-{2-[5-Bromo-2-(2,4-difluorobenzyloxy)phenyl]-5-methylpyrrol-1-yl}-5-trifluoromethylbenzoic acid

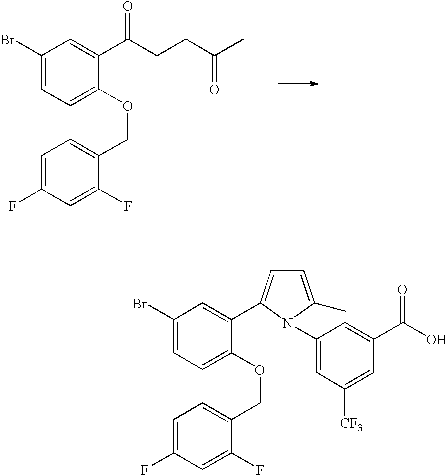

Procedure as for 3-{2-[5-chloro-2-(benzyloxy)-phenyl]-5-methylpyrrol-1-yl}-6-chloro-benzoic acid methyl ester using the appropriate amine, to give the title compound (44 mg, 16%).

LC/MS t=4.33 min [MH+] 566/568.

### Example 62

### 3-{2-[5-Bromo-2-(2,4-difluorobenzyloxy)-phenyl]-5-methylpyrrol-1-yl}-6-fluorobenzoic acid

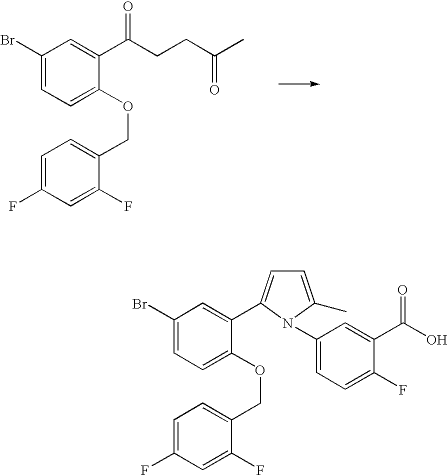

Procedure as for 3-{2-[5-chloro-2-(benzyloxy)-phenyl]-5-methylpyrrol-1-yl}-6-chloro-benzoic acid methyl ester using the appropriate amine, to give the title compound (40 mg, 15%).

¹H NMR (400 MHz, CDCl₃) 2.13 (3H, s), 4.77 (2H, s), 6.12 (1H, d, J=3 Hz), 6.26 (1H, d, J=3 Hz), 6.60 (1H, d, J=8 Hz), 6.76-6.84 (2H, m), 6.97-7.06 (2H, m), 7.08-7.15 (1H, m), 7.24 (1H, d, J=2 Hz), 7.37 (1H, d, J=2 Hz), 7.67 (1H, dd, J=2, 7 Hz).

LC/MS t=4.10 min [MH+] 516/518.

### Example 63

### 3-{2-[5-Bromo-2-(2,4difluorobenzyloxy)-phenyl]-5-methylpyrrol-1-yl}-4-fluorobenzoic acid

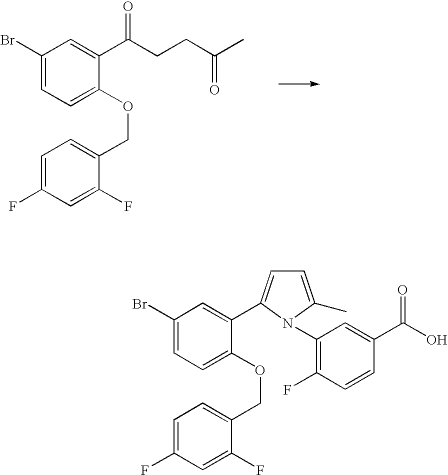

Procedure as for 3-{2-[5-Chloro-2-(benzyloxy)-phenyl]-5-methylpyrrol-1-yl}-6-chloro-benzoic acid methyl ester using the appropriate amine, to give the title compound (53 mg, 20%).

¹H NMR(400 MHz, CDCl₃) 2.10 (3H, s), 4.83 (2H, s), 6.15 (1H, d, J=3 Hz), 6.32 (1H, d, J=3 Hz), 6.56 (1H, d, J=9 Hz), 6.73 (2H, m), 6.99-7.08 (1H, m), 7.21 (1H, dd, J=2, 9 Hz), 7.85-7.17 (1H, m), 7.35 (1H, d, J=2 Hz), 7.77 (1H, dd, J=2, 7 Hz), 7.98-8.05 (1H, m).

LC/MS t=4.06 min [MH+] 516/518.

### Example 64

### 3-{2-[5-Bromo-2-(2,4-difluorobenzyloxy)-phenyl]-5-methylpyrrol-1-yl}-2,5,6-trifluorobenzoic acid

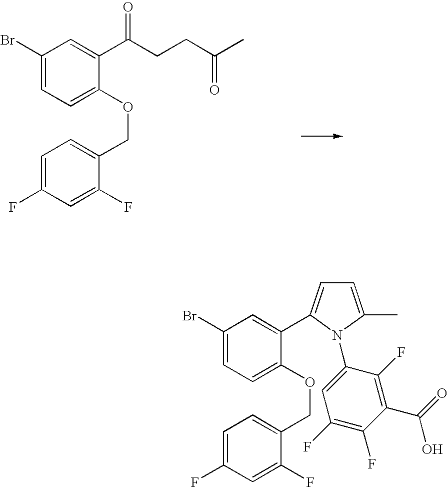

Procedure as for 3-{2-[5-chloro-2-(benzyloxy)-phenyl]-5-methylpyrrol-1-yl}-6-chloro-benzoic acid methyl ester using the appropriate amine, with 4 Å molecular sieve (˜0.5 g) to give the title compound (70 mg, 25%).

LC/MS t=4.65 min [MH+] 552/554.

### Example 65

### 3-{2-[5-Bromo-2-(2,4-difluorobenzyloxy)-phenyl]-5-methylpyrrol-1-yl}-5-Amino-benzoic acid

### a) 3-{2-[5-Bromo-2-(2,4-difluorobenzyloxy)-phenyl]-5-methylpyrrol-1-yl}-5-amino-benzoic acid methyl ester

Procedure as for 3-{2-[5-chloro-2-(benzyloxy)-phenyl]-5-methylpyrrol-1-yl}-6-chloro-benzoic acid methyl ester using the appropriate amine, with 4 Å molecular sieve (˜0.5 g) to give the title compound (73 mg, 70%).

¹H NMR (400 MHz, CDCl₃) 2.15 (3H, s), 3.82 (3H, s), 4.99 (2H, s), 6.08 (1H, d J=3 Hz), 6.27 (1H, d, J=3 Hz), 6.42 (1H, t, J=2, 4 Hz), 6.58 (1H, d, J=8 Hz), 6.75-6.84 (2H, m), 7.01-7.09 (1H, m), 7.11 (1H, s), 7.18-7.24 (2H,m), 7.33 (1H, d J=2 Hz).

LC/MS t=3.96 min [MH+] 527/529.

### b) 3-{2-[5-Bromo-2-(2,4-difluorobenzyloxy)-phenyl]-5-methylpyrrol-1-yl}-5-Amino-benzoic acid

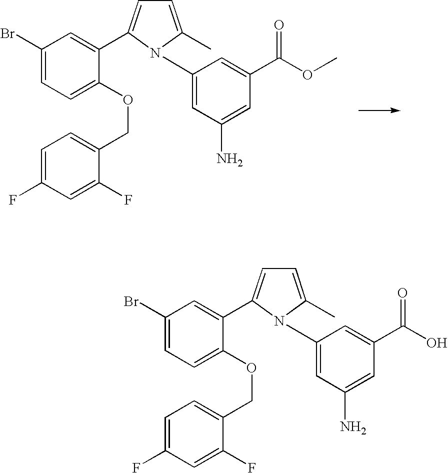

Procedure as for 3-{2-[5-chloro-2-(benzyloxy)-phenyl]-5-methylpyrrol-1-yl}-6-chloro-benzoic acid methyl ester, to give the title compound (48 mg, 33%).

¹H NMR (400 MHz, CDCl₃) 2.16 (3H, s), 4.80 (2H, s), 6.09 (1H, d, J=3 Hz), 6.28 (1H, d, J=3 Hz), 6.45 (1H, s), 6.59 (1H, d, J=9 Hz), 6.73-6.84 (2H, m), 7.02-7.10 (1H, m), 7.17 (1H, s), 7.19-7.29 (2H, m), 7.34 (1H, d, J=2 Hz).

LC/MS t=3.77 min [MH+] 513/515.

### Example 66

### 3-{2-[5-Bromo-2-(2,4-difluorobenzyloxy)-phenyl]-5-methylpyrrol-1-yl}-5-(2-oxopyrrolidin-1-yl)-benzoic acid

### a) 3-Nitro-5-{2-oxo-pyrrolidin-1-yl)-benzoic acid methyl ester

3-Bromo-5-methyl-benzoic acid methyl ester (2.48g, 10 mmol) (South et al., WO0187854), 2-pyrrolidinone (0.89 ml, 12 mmol), caesium carbonate (4.8g, 14 mmol), palladium bis(dibenzylideneacetone) (190 mg, 0.2 mmol), 9,9-dimethyl-4,5-bis(diphenylphosphino)xanthene (400 mg, 0.7 mmol), and caesium carbonate (4.8g, 14.8 mmol) were heated at reflux, under nitrogen for 3 hours. The reaction mixture was then cooled and filtered through Celite® and washed through with CH₂Cl₂ (100 ml). The mixture was concentrated in vacuo, and the residue was purified by chromatography on silica gel with isohexane/Et₂O (20-80%) then MeOH/Et₂O (98%) as eluant, to give the title compound (2.0g, 76%).

¹H-NMR (400 MHz, CDCl₃) 2.36 (2H, dt, J=8 Hz), 2.70 (2H, t, J=8 Hz), 3.97 (2H, t, J=8 Hz), 3.99 (3H, s), 8.55 (1H, t, J=2 Hz), 8.62 (1H, t, J=2 Hz), 8.86 (1H, t, J=2 Hz).

LC/MS t=2.78 min [MH⁺] 265.

### b) 3-Amino-5-(2-oxo-pyrrolidin-1-yl)-benzoic acid methyl ester

3-Nitro-5-(2-oxo-pyrrolidin-1-yl)-benzoic acid methyl ester (2.0g, 7.6 mmol) and Raney nickel (0.5g) in methanol (70 ml) were stirred under a hydrogen atmosphere at room temperature for 3 hours. The mixture was cooled and the catalyst was filtered off through a pad of Celite^{□} and washed through with MeOH/CH₂Cl₂ (2.5:1, 1L). The mixture was concentrated in vacuo to yield the title compound as a white solid (1.6g, 90%).

¹H-NMR (400 MHz, DMSO) 2.03 (2H, dt, J=8 Hz), 2.47 (2H, t, J=8 Hz), 3.77 (2H, t, J=8 Hz), 3.80 (3H, s), 5.39 (2H, broad s), 6.96 (1H, s), 7.14 (1H, s), 7.36 (1H, s).

LC/MS t=2.19 min [MH⁺] 235.

### c) 3-{2-[5-Bromo-2-(2,4-difluorobenzyloxy)-phenyl]-5-methylpyrrol-1-yl}-5-(2-oxopyrrolidin-1-yl)-benzoic acid methyl ester

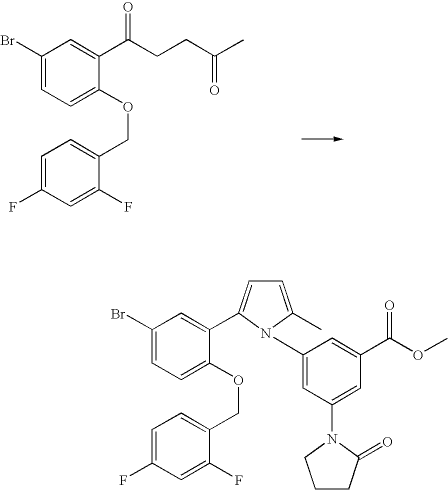

1-[5-Bromo-2-(2,4-difluorobenzyloxy)-phenyl]-pentane-1,4-dione (200 mg, 0.5 mmol), was treated with 3-amino-5-(2-oxopyrrolidin-1-yl)-benzoic acid methyl ester (1 17 mg, 0.5 mmol), and p-toluenesulfonic acid (˜50 mg) and 4 Å molecular sieve powder (˜0.5g) in N-methylpyrrolidinone(3 ml). The reaction mixture was then heated at 180° C. over 18 hrs under nitrogen. The mixture was cooled, concentrated to an oil at 80° C., diluted with EtOAc (7 ml) and filtered through Celite®, washing through with EtOAc (1 0 ml). The filtrate was then washed with brine, dried over MgSO₄, filtered and evaporated. The residue was purified by chromatography on silica gel with an isohexane/EtOAc gradiant system giving the title compound (60 mg, 20%).

¹H NMR (400 MHz, CDCl₃) 2.07-2.16 (2H, m), 2.18 (3H, s), 2.57 (2H, t J=8 Hz), 3.64 (2H, t, J=8 Hz), 3.85 (3H, s), 4.77 (2H, s), 6.12 (1H, d, J=3 Hz), 6.28 (1H, d, J=3 Hz), 6.56 (1H, d, J=8.5 Hz), 6.73-6.83 (2H, m), 6.93-7.25 (1H, m), 7.22 (1H, dd, J=2, 8.5 Hz), 7.37 (1H, d, J=2 Hz), 7.46 (1H, s), 7.61 (1H, s), 8.09 (1H, s).

LC/MS t=4.00 min [MH+] 595/597.

### d) 3-{2-[5-Bromo-2-(2,4-difluorobenzyloxy)-phenyl]-5-methylpyrrol-1-yl}-5-(2-oxopyrrolidin-1-yl)-benzoic acid

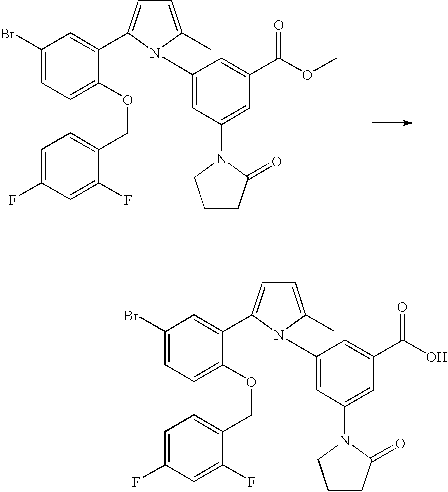

Procedure as for 3-{[2-[5-chloro-2-(benzyloxy)-phenyl]-5-methylpyrrol-1-yl}-6-chlorobenzoic acid, to give the title compound (50 mg, 86%).

¹H NMR (400 MHz, CDCl₃) 2.08-2.17 (2H, m), 2.19 (3H, s), 2.58 (2H, t, J=8 Hz), 3.66 (2H, t, =8 Hz), 4.78 (2H, s), 6.12 (1H, d, J=3 Hz), 6.28 (1H, d, J=3 Hz), 6.56 (1H, d, J=8.5 Hz), 6.71-6.83 (2H, m), 6.96-7.03 (1H, m), 7.22 (1H, dd, J=2, 8.4 Hz), 7.38 (1H, d, J=2 Hz), 7.50 (1H, s), 7.69 (1H, s), 8.10 (1H, s).

LC/MS t=3.84 min [MH+] 581/583.

### Example 67

### 3-{2-[5-Bromo-2-(cyclohexylmethoxy)-phenyl]-5-methyl-pyrrol-1-yl}-benzoic acid

### a) 3-{2-[5-Bromo-2-(cyclohexylmethoxy)-phenyl]-5-methyl-pyrrol-1-yl}-benzoic acid ethyl ester

Cyclohexylmethyl bromide (146 mg, 0.83 mmol) was added to 3-[2-(5-bromo-2-hydroxy-phenyl)-5-methyl-pyrrol-1-yl)--benzoic acid ethyl ester (220 mg, 0.55 mmol) and K₂CO₃ (152 mg, 1.1 mmol) in DMF (2 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel using isohexane/EtOAc (5%) as eluant, to give the title compound (179 mg, 66%).

¹H-NMR (400 MHz, CDCl₃) 0.79-0.92 (2H, m), 1.08-1.29 (3H, m), 1.38 (3H, t, J=7 Hz), 1.60-1.73 (6H, m), 2.19 (3H, s), 3.40 (2H, d, J=7 Hz), 4.36 (2H, q, J=7 Hz), 6.11 (1H, d, J=3 Hz), 6.28 (1H, d, J=3 Hz), 6.54 (1H, d, J=9 Hz), 7.15-7.22 (2H, m), 7.25 (1H, d, J=3 Hz), 7.32 (1H, t, J=8 Hz), 7.83 (1H, t, J=1 Hz), 7.92 (1H, dt, J=1 Hz, 8 Hz).

LC/MS t=4.51 min [MH⁺]=496.

### b) 3-{2-[5-Bromo-2-(cyclohexylmethoxy)-phenyl]-5-methyl-pyrrol-1-yl}-benzoic acid

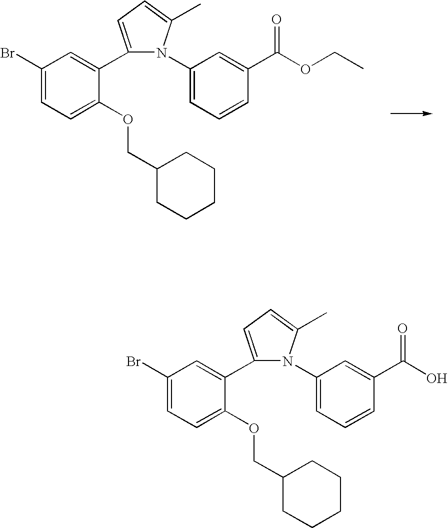

3-{2-[5-Bromo-2-(cyclohexylmethoxy)-phenyl]-5-methyl-pyrrol-1-yl}-benzoic acid ethyl ester (150 mg, 0.3 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (129 mg, 91%).

¹H-NMR (400 MHz, CDCl₃) 0.79-0.92 (2H, m), 1.09-1.29 (3H, m), 1.59-1.73 (6H, m), 2.19 (3H, s), 3.41 (2H, d, J=7 Hz), 6.11 (1H, d, J=3 Hz), 6.28 (1H, d, J=3 Hz), 6.54 (1H, d, J=9 Hz), 7.19-7.25 (3H, m), 7.35 (1H, t, J=8 Hz), 7.91 (1H, broad s), 7.98 (1H, d, J=8 Hz).

LC/MS t=4.33 min[MH⁺]=468.

### Example 68

### 3-{2-[5-Methanesulfonyl-2-(benzyloxy)-phenyl]-5-methyl-pyrrol-1-yl}-benzoic acid

### a) 5-Methanesulfonyl-2-(4-methoxy-benzyloxy)-benzaldehyde

4-Methoxybenzyl chloride (4.09g, 0.026 mol) was added to 2-hydroxy-5methanesulfonyl-benzaldehyde (3.48g, 0.017 mol) {prepared via the method of Suzuki et al, Chem. Pharm. Bull., 1989, 31 (5),1751) and K₂CO₃ (4.8 g, 0.035 mol) in DMF (35 ml). The mixture was heated to 60° C. for 3 hours. The reaction mixture was quenched with water (250 ml) and washed with EtOAc (2×250 ml). The organic extracts were combined and washed with brine (150 ml), dried (MgSO₄) filtered and concentrated to give the title compound (6.5g, 100%).

¹H-NMR (400 MHz, d6-DMSO) F5778 3.77 (3H, s), 5.35 (2H, s), 6.96 (2H, d, J=9 Hz), 7.47 (2H, d, J=9 Hz), 7.59 (1H, d, J=10 Hz), 8.14-8.19 (2H, m).

LC/MS t=2.94 min [MNH₄⁺]=338.

### b) 1-[5-Methanesulfonyl-2-(4-methoxy-benzyloxy)-phenyl]-pentane-1,4-dione

Methyl vinyl ketone (1.72 ml, 20 mmol) and 3-ethyl-5-(2-hydroxyethyl)-4-methylthiazolium bromide (1.54g, 6 mmol) were added to 5-methanesulfonyl-2-(4-methoxy-benzyloxy)-benzaldehyde (6.5g, 20 mmol) in EtOH (5.5 ml) and triethylamine (8.5 ml, 60 mmol). The mixture was heated at 100° C. for 18 hours. The reaction mixture was quenched with saturated NH₄Cl solution (300 ml) and washed with EtOAc (2×250 ml). The organic extracts were combined and washed with saturated NaHCO₃ solution (250 ml) and brine (200 ml), dried (MgSO₄) filtered and concentrated. The crude product was purified by chromatography on silica gel (50% EtOAc/iso-hexane) to give the title compound (2.92 g, 37%).

¹H-NMR (400 MHz, d6-DMSO) 2.09 (3H, s), 2.73 (2H, t, J=6 Hz), 3.10 (2H, t, J=6 Hz), 3.77 (3H, s), 5.30 (2H, s), 6.97 (2H, d, J=9 Hz), 7.47 (2H, d, J=9 Hz), 7.53 (1H, d, J=9 Hz), 8.04-8.08 (2H, m).

LC/MS t=2.92 min [MNH⁻]=389.

### c) 3-{2-[5-Methanesulfonyl-2-(hydroxy)-phenyl]-5-methyl-pyrrol-1-yl}-benzoic acid ethyl ester

1-[5Methanesulfonyl-2-(4-methoxy-benzyloxy)-phenyl]-pentane-1,4-dione (2.91g, 7.5 mmol), ethyl-3-aminobenzoate (1.6 ml, 10.7 mmol) and para-toluenesulfonic acid (0.22g, 1.2 mmol) were heated at reflux in toluene (75 ml) for 16 hours. Upon cooling, the mixture was concentrated. The residue was purified by chromatography on silica gel with isohexane/EtOAc (40%) as eluant, to give the title compound (2.22g, 74%).

¹H-NMR (400 MHz, CDCl₃) 1.37 (3H, t, J=7 Hz), 2.18 (3H, s), 2.68 (3H, s), 4.35 (2H, q, J=7 Hz), 6.21 (1H, d, J=3 Hz), 6.44 (1H, d, J=3 Hz), 6.56 (1H, s), 7.02 (1H, d, J=9 Hz), 7.28-7.30 (2H, m), 7.43 (1H, t, J=8 Hz), 7.63 (1H, dd, J=2 Hz, 9 Hz), 7.77 (1H, t, J=1 Hz), 7.99 (1H, dt, J=1 Hz, 8 Hz).

LC/MS t=3.17 min [MH⁺]=400.

### d) 3-{2-[5-Methanesulfonyl-2-(benzyloxy)-phenyl]-5-methyl-pyrrol-1-yl}-benzoic acid ethyl ester

Benzyl bromide (0.089 ml, 0.75 mmol) was added to 3-{2-[5-Methanesulfonyl-2-(hydroxy)-phenyl]-5-methyl-pyrrol-1-yl}-benzoic acid ethyl ester (200 mg, 0.50 mmol) and K₂CO₃ (138 mg, 1.0 mmol) in DMF (2 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (30%) as eluant, to give the title compound (218 mg, 89%).

¹H-NMR (400 MHz, CDCl₃) 1.31 (3H, t, J=7 Hz), 2.17 (3H, s), 2.88 (3H, s), 4.28 (2H, q, J=7 Hz), 4.91 (2H, s), 6.15 (1H, d, J=3 Hz), 6.42 (1H, d, J=3 Hz), 6.79 (1H, d, J=9 Hz), 7.10-7.15 (2H, m), 7.16-7.19 (1H, m), 7.28-7.34 (4H, m), 7.65 (1H, dd, J=2 Hz, 8 Hz), 7.70 (1H, d, J=2 Hz), 7.73 (1H, t, J=1 Hz), 7.99 (1H, d, J=8 Hz).

LC/MS t=3.68 min [MH⁺]=490.

### e) 3-{2-[5-Methanesulfonyl-2-(benzyloxy)-phenyl]-5-methyl-pyrrol-1-yl}-benzoic acid

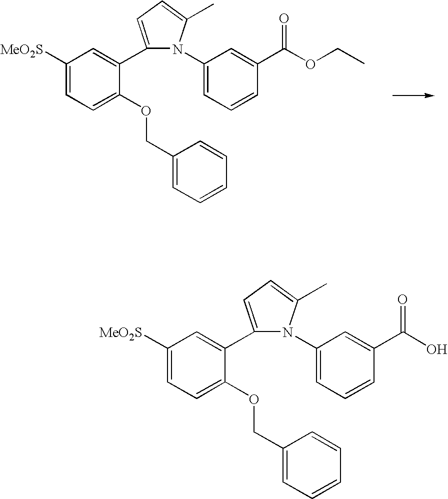

3-{[2-[5-Methanesulfonyl-2-(benzyloxy)-phenyl]-5-methyl-pyrrol-1-yl}-benzoic acid ethyl ester (200 mg, 0.4 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (177 mg, 95%).

¹H-NMR (400 MHz, CDCl₃) 2.17 (3H, s), 2.90 (3H, s), 4.91 (2H, s), 6.17 (1H, d, J=3 Hz), 6.42 (1H, d, J=3 Hz), 6.80 (1H, d, J=9 Hz), 7.10-7.14 (2H, m), 7.20-7.25 (1H, m), 7.28-7.38 (4H, m), 7.66 (1H, dd, J=2 Hz, 8 Hz), 7.72 (1H, d, J=2 Hz), 7.75 (1H, t, J=1Hz), 7.97 (1H, d, J=8 Hz).

LC/MS t=3.40 min [MH⁺=462.

### Example 69

### 3-{2-[5-Methanesulfonyl-2-(4-chloro-benzyloxy)-phenyl]-5-methyl-pyrrol-1-yl}-benzoic acid

### a) 3-{2-[5-Methanesulfonyl-2-(4-chloro-benzyloxy)-phenyl]-5-methyl-pyrrol-1-yl}-benzoic acid ethyl ester

4-Chloro-benzyl bromide (154 mg, 0.75 mmol) was added to 3-{2-[5-methanesulfonyl-2-(hydroxy)-phenyl]-5-methyl-pyrrol-1-yl}-benzoic acid ethyl ester (200 mg, 0.50 mmol) and K₂CO₃ (1 38 mg, 1.0 mmol) in DMF (2 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (30%) as eluant, to give the title compound (205 mg, 78%).

¹H-NMR (400 MHz, CDCl₃) 1.31 (3H, t, J=7 Hz), 2.17 (3H, s), 2.89 (3H, s), 4.29 (2H, q, J=7 Hz), 4.87 (2H, s), 6.16 (1H, d, J=3 Hz), 6.41 (1H, d, J=3 Hz), 6.77 (1H, d, J=9 Hz), 7.05 (2H, d, J=8 Hz), 7.16-7.19 (1H, m), 7.28-7.34 (3H, m), 7.67 (1H, dd, J=2 Hz, 8 Hz), 7.70-7.73 (2H, m), 7.92 (1H, d, J=8 Hz).

LC/MS t=3.84 [MH⁺]=524/526.

### b) 3-{2-[5-Methanesulfonyl-2-(4-chloro-benzyloxy)-phenyl]-5-methyl-pyrrol-1-yl}-benzoic acid

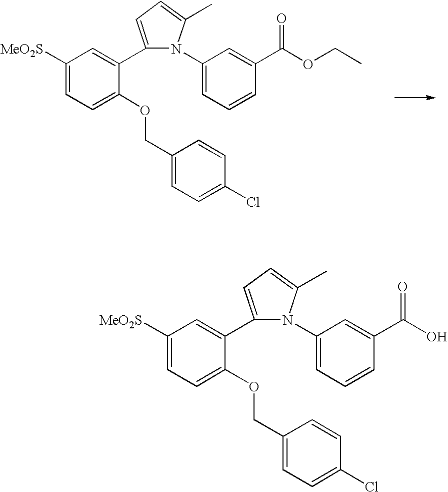

3-{2-[5-methanesulfonyl-2-(4-chloro-benzyloxy)-phenyl]-5-methyl-pyrrol-1-yl}-benzoic acid ethyl ester (200 mg, 0.4 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (164 mg, 87%).

¹H-NMR (400 MHz, CDCl₃) 2.18 (3H, s), 2.90 (3H, s), 4.86 (2H, s), 6.16 (1H, d, J=3 Hz), 6.41 (1H, d, J=3 Hz), 6.77 (1H, d, J=9 Hz), 7.07 (2H, d, J=8 Hz), 7.20-7.24 (1H, m), 7.29 (2H, d, J=8 Hz), 7.36 (1H, t, J=8 Hz), 7.68 (1H, dd, J=2 Hz, 8 Hz), 7.72 (2H, d, J=2 Hz), 7.98 (1H, d, J=8 Hz).

LC/MS t=3.59 min [MH⁺]=496/498.

### Example 70

### 3-{2-[5-Methanesulfonyl-2-(4-fluoro-benzyloxy)-phenyl]-5-methyl-pyrrol-1-yl}-benzoic acid

### a) 3-{2-[5-Methanesulfonyl-2-(4-fluoro-benzyloxy)-phenyl]-5-methyl-pyrrol-1-yl}-benzoic acid ethyl ester

4-Fluoro-benzyl bromide (142 mg, 0.75 mmol) was added to 3-{2-[5-methanesulfonyl-2-(hydroxy)-phenyl]-5-methyl-pyrrol-1-yl}-benzoic acid ethyl ester (200 mg, 0.50 mmol) and K₂CO₃ (138 mg, 1.0 mmol) in DMF (2 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel, with isohexane/EtOAc (30%) as eluant, to give the title compound (229 mg, 90%).

¹H-NMR (400 MHz, CDCl₃) 1.31 (3H, t, J=7 Hz), 2.17 (3H, s), 2.88 (3H, s), 4.29 (2H, q, J=7 Hz), 4.86 (2H, s), 6.14 (1H, d, J=3 Hz), 6.41 (1H, d, J=3 Hz), 6.79 (1H, d, J=9 Hz), 6.99-7.05 (2H, m), 7.08-7.14 (2H, m), 7.15-7.17 (1H, m), 7.32 (1H, t, J=8 Hz), 7.65-7.71 (3H, m), 7.92 (1H, d, J=8 Hz).

LC/MS t=3.70 min [MNH₄⁺]=525.

### b) 3-{2-[5-Methanesulfonyl-2-(4-fluoro-benzyloxy)-phenyl]-5-methyl-pyrrol-1-yl}-benzoic acid

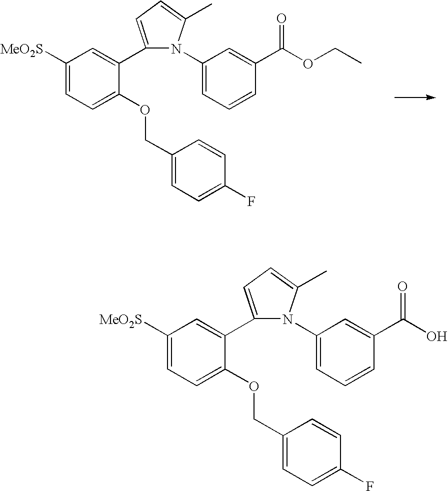

3-{2-[5-Methanesulfonyl-2-(4-fluoro-benzyloxy)-phenyl]-5-methyl-pyrrol-1-yl}-benzoic acid ethyl ester (200 mg, 0.4 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (173 mg, 92%).

¹H-NMR (400 MHz, CDCl₃) 2.18 (3H, s), 2.90 (3H, s), 4.87 (2H, s), 6.15 (1H, d, J=3 Hz), 6.41 (1H, d, J=3 Hz), 6.80 (1H, d, J=9 Hz), 6.99-7.05 (2H, m), 7.09-7.14 (2H, m), 7.19-7.23 (1H, m), 7.36 (1H, t, J=8 Hz), 7.67 (1H, dd, J=2 Hz, 8 Hz), 7.72 (2H, d, J=2 Hz), 7.97 (1H, d, J=8 Hz).

LC/MS t=3.42 min [MH⁺]=480.

### Example 71

### 3-{2-[5-Methanesulfonyl-2-(2-chloro-4-fluoro-benzyloxy)-phenyl]-5-methyl-pyrrol-1-yl}-benzoic acid

### a) 3-{2-[5-Methanesulfonyl-2-(2-chloro)-4-fluoro-benzyloxy)-phenyl]-5-methyl-pyrrol-1-yl}-benzoic acid ethyl ester

2-Chloro-4-fluoro-benzyl bromide (168 mg, 0.75 mmol) was added to 3-{2-[5-methanesulfonyl-2-(hydroxy)-phenyl]-5-methyl-pyrrol-1-yl}-benzoic acid ethyl ester (200 mg, 0.50 mmol) and K₂CO₃ (138 mg, 11.0 mmol) in DMF (2 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (30%) as eluant, to give the title compound (248 mg, 91%).

¹H-NMR (400 MHz, CDCl₃) 1.31 (3H, t, J=7 Hz), 2.18 (3H, s), 2.89 (3H, s), 4.29 (2H, q, J=7 Hz), 4.93 (2H, s), 6.16 (1H, d, J=3 Hz), 6.42 (1H, d, J=3 Hz), 6.79 (1H, d, J=9 Hz),6.94 (1H, ddd, J=2 Hz, 8 Hz), 7.00-7.16 (1H, m), 7.13 (1H, dd, J=2 Hz, 8 Hz), 7.18-7.22 (1H, m), 7.34 (1H, t, J=8 Hz), 7.67-7.73 (3H, m), 7.93 (1H, d, J=8 Hz).

LC/MS t=3.84 min [MNH₄⁺]=559/561.

### b) 3-{2-[6-Methanesulfonyl-2-(2-chloro 4-fluoro-benzyloxy)-phenyl]-5-methyl-pyrrol-1-yl}-benzoic acid

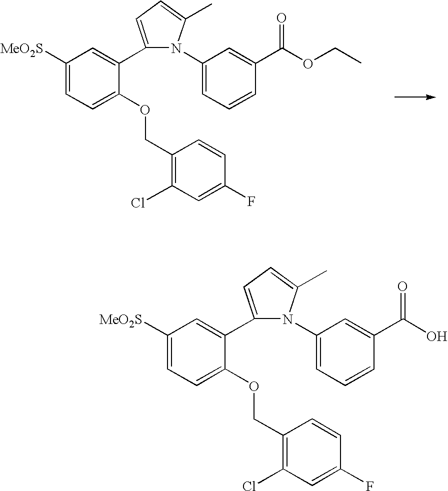

3-{2-[5-Methanesulfonyl-2-(2-chloro-4-fluoro-benzyloxy)-phenyl]-5-methyl-pyrrol-1-yl}-benzoic acid ethyl ester (200 mg, 0.4 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (170 mg, 90%).

¹H-NMR (400 MHz, CDCl₃) 2.18 (3H, s), 2.90 (3H, s), 4.93 (2H, s), 6.17 (1H, d, J=3 Hz), 6.42 (1H, d, J=3 Hz), 6.80 (1H, d, J=9 Hz), 6.94 (1H, ddd, J=2 Hz, 8 Hz), 7.03-7.08 (1H, m), 7.12 (1H, dd, J=2 Hz, 8 Hz), 7.25-7.27 (1H, m), 7.39 (1H, t, J=8 Hz), 7.69-7.75 (3H, m), 7.98 (1H, d, J=8 Hz).

LC/MS t=3.57 min [MH⁺]=514/516.

### Example 72

### 3-{2-[5-Methanesulfonyl-2-(2,4-difluoro-benzyloxy)-phenyl]-5-methyl-pyrrol-1-yl}-benzoic acid

### a) 3-{2-[5-Methanesulfonyl-2-(2,4-difluoro-benzyloxy)-phenyl]-5-methyl-pyrrol-1-yl}-benzoic acid ethyl ester

2,4-Difluoro-benzyl bromide (156 mg, 0.75 mmol) was added to 3-{2-[5-methanesulfonyl-2-(hydroxy)-phenyl]-5-methyl-pyrrol-1-yl}-benzoic acid ethyl ester (200 mg, 0.50 mmol) and K₂CO₃ (1 38 mg, 11.0 mmol) in DMF (2 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (30%) as eluant, to give the title compound (233 mg, 88%).

¹H-NMR (400 MHz, CDCl₃) 1.32 (3H, t, J=7 Hz), 2.17 (3H, s), 2.88 (3H, s), 4.30 (2H, q, J=7 Hz), 4.92 (2H, s), 6.14 (1H, d, J=3 Hz), 6.40 (1H, d, J=3 Hz), 6.80-6.87 (3H, m), 7.05-7.12 (1H, m), 7.18 (1H, d, J=8 Hz), 7.34 (1H, t, J=8 Hz), 7.68-7.72 (3H, m), 7.93 (1H, d, J=8 Hz).

LC/MS t=3.71 min [MH⁺]=526.

### b) 3-{2-[5-Methanesulfonyl-2-(2,4-fluoro-benzyloxy)-phenyl]-5-methyl-pyrrol-1-yl}-benzoic acid

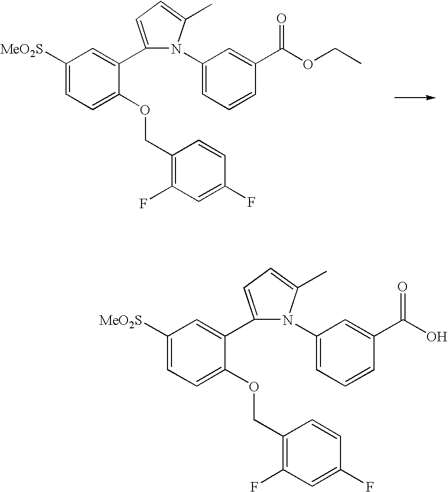

3-{2-[5-Methanesulfonyl-2-(2,4-difluoro-benzyloxy)-phenyl]-5-methyl-pyrrol-1-yl}-benzoic acid ethyl ester (200 mg, 0.4 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (168 mg, 89%).

¹H-NMR (400 MHz, CDCl₃) 2.18 (3H, s), 2.90 (3H, s), 4.92 (2H, s), 6.16 (1H, d, J=3 Hz), 6.40 (1H, d, J=3 Hz), 6.80-6.87 (3H, m), 7.05-7.12 (1H, m), 7.18 (1H, d, J=8 Hz), 7.36 (1H, t, J=8 Hz), 7.68-7.73 (3H, m), 7.99 (1H, d, J=8 Hz).

LC/MS t=3.44 min [MH⁺]=498.

### Example 73

### 3-{2-[5-Trifluoromethyl-2-(benzyloxy)-phenyl]-5-methyl-pyrrol-1-yl]-benzoic acid

### a) 2-Benzyloxy-5-trifluoromethyl-benzaldehyde

2-Hydroxy-5-trifluoromethyl-benzaldehyde (prepared via the procedure of Schäfer, Synthesis 2001, 15, 2259-2262) (0.5g, 2.63 mmol), benzyl bromide (0.313 ml, 3.95 mmol) and potassium carbonate (0.727g, 5.26 mmol) were heated in DMF (5 ml) at 50° C. in a nitrogen atmosphere for 1 hour. Upon cooling the reaction mixture was diluted with EtOAc and washed with salt. NH₄Cl. The organic layer was extracted and the aqueous layer washed with 3×EtOAc. The combined organic extracts were then washed with brine and dried over MgSO₄, filtered and concentrated in vacuo. The resultant residue was purified by chromatography on silica gel eluting with 10% EtOAc/iso-hexane this yielded the title compound as a clear solid (0.14g, 19%).

¹H-NMR (400 MHz, CDCl₃) 5.27 (2H, s), 7.15 (1H, d, J=9 Hz), 7.32-7.48 (5H, m), 7.78 (1H, dd, J=3 Hz, 9 Hz) 8.12 (1H, d, J=3 Hz) 10.60 (1H, s).

LC/MS t=3.59 min.

### b) 1-[5-Trifluoromethyl-2-(benzyloxy)-phenyl]-pentane-1,4-dione

2-Benzyloxy-5-trifluoromethyl-benzaldehyde (0.4g, 50% purity, 0.714 mmol), triethylamine (0.3 ml, 2.14 mmol), methyl vinyl ketone (0.061 ml, 0.728 mmol) and 3-ethyl-5-(2-hydroxyethyl)-4-ethylthiazolium bromide (0.054g, 0.214 mmol) were refluxed in EtOH (1.5 ml) under a nitrogen atmosphere for 22 hours. Upon cooling the reaction mixture was diluted with EtOAc and washed with saturated NH₄Cl. The organic layer was extracted and the aqueous layer washed with 3×EtOAc. The combined organic extracts were then washed with saturated NaHCO₃ and brine and then dried over MgSO₄, filtered and concentrated in vacuo. The residue was purified by chromatography on silica gel eluting with 10% EtOAc/iso-hexane. This yielded the title compound as an off white solid (0.057g, 23%).

¹H-NMR (400 MHz, CDCl₃) 2.19 (3H, s), 2.80 (2H, t, J=6 Hz), 3.26 (2H, t, J=6 Hz), 5.23 (2H, s), 7.10 (1H, d, J=9 Hz), 7.35-7.46 (5H, m), 7.68 (1H, dd, J=3 Hz, 9 Hz), 8.02 (1H, d, J=3 Hz).

LC/MS t=3.51 min.

### c) 3-{2-[5-Trifluoromethyl-2-(benzyloxy)-phenyl]-5-methyl-pyrrol-1-yl}-benzoic acid ethyl ester

1-[5-Trifluoromethyl-2-(benzyloxy)-phenyl]-pentane-1,4-dione (1 g, 2.86 mmol) and ethyl-3-aminobenzoate (0.51 ml, 3.43 mmol) were heated in a sealed vessel at 150° C. for 26 hours. Upon cooling the residue was purified by chromatography on silica gel eluting with 10% EtOAc/iso-hexane. This yielded the title compound as a yellow oil (0.77g, 56%).

¹H-NMR (400 MHz, CDCl₃) 1.29 (3H, t, J=7 Hz), 2.17 (3H, s), 4.28 (2H, q, J=7 Hz), 4.82 (2H, s), 6.15 (1H, d, J=3 Hz), 6.37 (1H, d, J=3 Hz), 6.70 (1H, d, J=9 Hz), 7.05-7.08 (3H, m), 7.13 (1H, dt, J=1Hz, 8 Hz), 7.28-7.35 (5H, m) 7.49 (1H, d, J=2 Hz), 7.72 (1H, t, J=1 Hz), 7.92 (1H, dt, J=1 Hz, 8 Hz).

LC/MS t=4.19 min [MH⁺] 480.

### d) 3-{2-[5-Trifluoromethyl-2-(benzyloxy)-phenyl]-5-methyl-pyrrol-1-yl}-benzoic acid

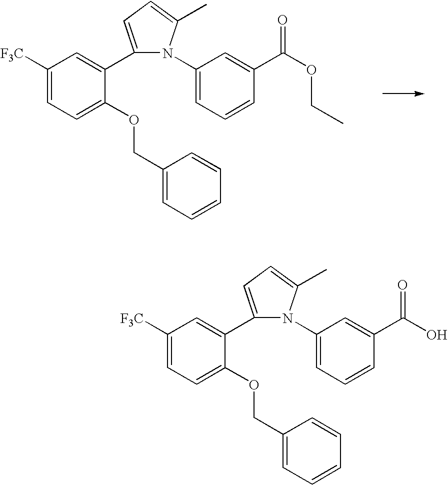

3-{2-[5-Trifluoromethyl-2-(benzyloxy)-phenyl]-5-methyl-pyrrol-1-yl}-benzoic acid ethyl ester (0.1g), 2M NaOH (3 ml) and EtOH (5 ml) were heated at 100° C. in a sealed vessel for 45 minutes. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound as a brown solid (0.090g, 96%).

¹H-NMR (400 MHz, CDCl₃) 2.18 (3H, s), 4.84 (2H, s), 6.16 (1H, d, J=3 Hz), 6.37 (1H, d, J=3 Hz), 6.72 (1H, d, J=9 Hz), 7.08 (2H, dd, J=1 Hz, 9 Hz), 7.17 (1H, broad d, J=9 Hz), 7.28-7.36 (5H, m), 7.47 (1H, d, J=2 Hz), 7.79 (1H, t, J=1 Hz), 7.96 (1H, dt, J=1 Hz, 8 Hz).

LC/MS t=3.92 min [MH⁺] 452.

### Example 74

### 3-{2-[5-Trifluoromethyl-2-(4-chloro-benzyloxy)-phenyl]-5-methyl-pyrrol-1-yl}-benzoic acid

### a) 3-{2-[5-Trifluoromethyl-2-(hydroxy)-phenyl]-5-methyl-pyrrol-1-yl}-benzoic acid ethyl ester

3-{2-[5-Trifluoromethyl-2-(benzyloxy)-phenyl]-5-methyl-pyrrol-1-yl}-benzoic acid ethyl ester (0.65g, 1.36 mmol), palladium on charcoal (10% containing 50% water) (0.13g, 20% w/w), ammonium formate (0.45g, 7.19 mmol) and EtOH (9 ml) were stirred at 60° C. under a nitrogen atmosphere for 1.5 hours. Upon cooling the mixture was filtered and the solvent removed in vacuo. The residue was purified by chromatography on silica gel eluting with 10% EtOAc/iso-hexane to yield the title compound as a yellow oil (0.513g, 97%)

¹H-NMR (400 MHz, CDCl₃) F7101 1.37 (3H, t, J=7 Hz), 2.17 (3H, s), 4.35 (2H, q, J=7 Hz), 6.18 (1H, d, J=3 Hz), 6.28 (1H, s), 6.40 (1H, d, J=3 Hz), 6.94 (1H, d, J=9 Hz), 6.99 (1H, d, J=2 Hz), 7.22 (1H, broad d, J=9 Hz), 7.31 (1H, dd, J=2 Hz, 9 Hz), 7.39 (1H, t, J=8 Hz), 7.79 (1H, t, J=1 Hz), 7.98 (1H, dt, J=0.5 Hz, 8 Hz).

LC/MS t=3.77 min [MH+] 390.

### b) 3-{2-[5-Trifluoromethyl-2-(4-chloro-benzyloxy)-phenyl]-5-methyl-pyrrol-1-yl}-benzoic acid ethyl ester

3-{2-[5-Trifluoromethyl-2-(hydroxy)-phenyl]-5-methyl-pyrrol-1-yl}-benzoic acid ethyl ester (0.085g, 0.219 mmol), 4-chlorobenzyl bromide (0.068g, 0.329 mmol) and potassium carbonate (0.061g, 0.438 mmol) were heated in DMF (2 ml) at 65° C. in a nitrogen atmosphere for 3.5 hours. Upon cooling the mixture was diluted with EtOAc and washed with 2× water. The organic layer was extracted and the aqueous layer washed with 3×EtOAc. The combined organics were then washed with brine and concentrated in vacuo. The residue was purified by chromatography using a Biotage^{□} 25S column eluting with 8% EtOAc/iso-hexane. This yielded the title compound as a clear oil (0.078g, 69%).

¹H-NMR (400 MHz, CDCl₃) 1.29 (3H, t, J=7 Hz), 2.17 (3H, s), 4.28 (2H, q, J=7 Hz), 4.77 (2H, s), 6.15 (1H, d, J=3 Hz), 6.35 (1H, d, J=3 Hz), 6.67 (1H, d, J=9 Hz), 7.00 (2H, d, J=9 Hz), 7.02 (1H, broad d, J=9 Hz), 7.27-7.39 (4H, m), 7.50 (1H, d, J=2 Hz), 7.70 (1H, t, J=1 Hz), 7.91 (1H, dt, J=0.5 Hz, 8 Hz).

LC/MS t=4.33 min [MH⁺] 514/516.

### c) 3-{2-[5-Trifluoromethyl-2-(4-chloro-benzyloxy)-phenyl]-5-methyl-pyrrol-1-yl}-benzoic acid

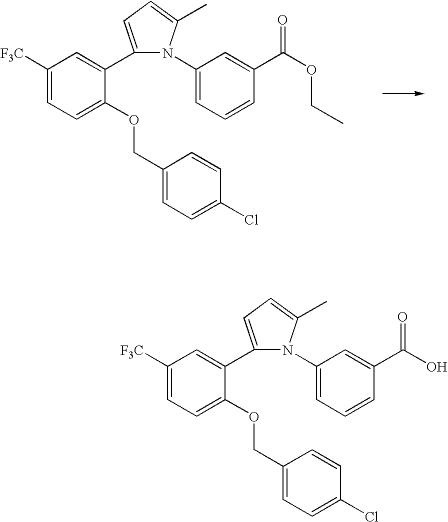

3-{2-[5-Trifluoromethyl-2-(4-chloro-benzyloxy)-phenyl]-5-methyl-pyrrol-1-yl}-benzoic acid ethyl ester (0.078g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound as a brown solid (0.085g, 100%+pyrolidine equiv.)

¹H-NMR (400 MHz, MeOD) 2.12 (3H, s), 4.89 (2H, s), 6.07 (1H, d, J=3 Hz), 6.29 (1H, d, J=3 Hz), 6.88 (1H, d, J=9 Hz), 7.07 (1H, broad d, J=8 Hz), 7.15 (2H, d, J=9 Hz), 7.26-7.37 (5H, m), 7.72 (1H, broad s), 7.93 (1H, d, J=8 Hz).

LC/MS t=4.09 min [MH⁺] 486/488.

### Example 75

### 3-{2-[5-Trifluoromethyl-2-(4-fluoro-benzyloxy)-phenyl]-5-methyl-pyrrol-1-yl}-benzoic acid

### a) 3-{2-[5-Trifluoromethyl-2-(4-fluoro-benzyloxy)-phenyl]-5-methyl-pyrrol-1-yl}-benzoic acid ethyl ester

3-{2-[5-Trifluoromethyl-2-(hydroxy)-phenyl]-5-methyl-pyrrol-1-yl}-benzoic acid ethyl ester (0.085g, 0.219 mmol), 4-fluorobenzyl bromide (0.0419, 0.329 mmol) and potassium carbonate (0.061g, 0.438 mmol) were heated in DMF (2 ml) at 65° C. in a nitrogen atmosphere for 3.5 hours. Upon cooling the mixture was diluted with EtOAc and washed with 2× water. The organic layer was extracted and the aqueous layer washed with 3×EtOAc. The combined organics were then washed with brine and concentrated in vacuo. The residue was purified by chromatography on silica gel eluting with 8% EtOAc/iso-hexane. This yielded the title compound as a clear oil (0.088g, 81%).

¹H-NMR (400 MHz, CDCl₃) 1.30 (3H, t, J=7 Hz), 2.16 (3H, s), 4.29 (2H, q, J=7 Hz), 4.76 (2H, s), 6.15 (1H, d, J=3 Hz), 6.35 (1H, d, J=3 Hz), 6.69 (1H, d, J=9 Hz), 6.97-7.13 (5H, m), 7.28 (1H, t, J=8 Hz), 7.35 (1H, dd, J=2 Hz,9 Hz), 7.49 (1H, d, J=2 Hz), 7.69 (1H, t, J=2 Hz), 7.91 (1H, dt, J=0.5 Hz, 8 Hz).

LC/MS t=4.21 min [MH⁺] 498.

### b) 3-{2-[5-Trifluoromethyl-2-(4-fluoro-benzyloxy)-phenyl]-5-methyl-pyrrol-1-yl}-benzoic acid

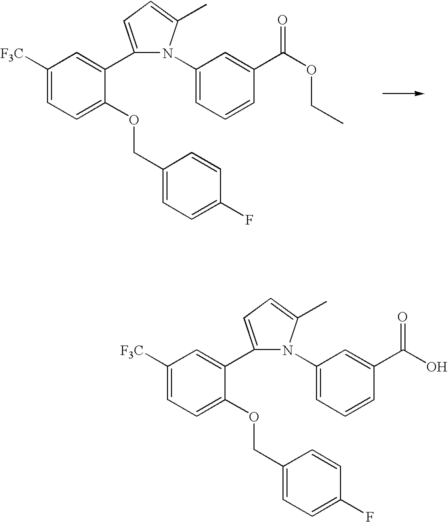

3-{2-[5-Trifluoromethyl-2-(4-fluoro-benzyloxy)-phenyl]-5-methyl-pyrrol-1-yl}-benzoic acid ethyl ester (0.088g), 2M NaOH (1 ml) and EtOH (2 ml) were heated at 100° C. in a sealed vessel for 45 minutes. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound as a brown solid (0.087g, 100%+pyrrolidine equivalent).

¹H-NMR (400 MHz, CDCl₃) 2.18 (3H, s), 4.79 (2H, s), 6.16 (1H, d, J=3 Hz), 6.36 (1H, d, J=3 Hz), 6.72 (1H, d, J=9 Hz), 6.97-7.03 (2H, m), 7.06-7.10 (2H, m), 7.14-7.18 (1H, m), 7.32 (1H, t, J=8 Hz), 7.36 (1H, dd, J=2 Hz, 9 Hz), 7.47 (1H, d, J=2 Hz), 7.76 (1H, t, J=1 Hz), 7.96 (1H, dt, J=0.5 Hz, 8 Hz).

LC/MS t=3.95 min [MH⁺] 470.

### Example 76

### 3-{2-[5-Trifluoromethyl-2-(2-chloro-4-fluoro-benzyloxy)-phenyl]-5-methyl-pyrrol-1-yl)--benzoic acid

### a) 3-{2-[5-Trifluoromethyl-2-(2-chloro-4-fluoro-benzyloxy)-phenyl]-5-methyl-pyrrol-1-yl}-benzoic acid ethyl ester

3-{2-[5-Trifluoromethyl-2-(hydroxy)-phenyl]-5-methyl-pyrrol-1-yl}-benzoic acid ethyl ester (0.085g, 0.219 mmol), 2-chloro-4-fluorobenzyl bromide (0.073g, 0.329 mmol) and potassium carbonate (0.061 g, 0.438 mmol) were heated in DMF (2 ml) at 65° C. in a nitrogen atmosphere for 3.5 hours. Upon cooling the mixture was diluted with EtOAc and washed with 2× water. The organic layer was extracted and the aqueous layer washed with 3×EtOAc. The combined organics were then washed with brine and concentrated in vacuo. The residue was purified by chromatography on silica gel eluting with 8% EtOAc/iso-hexane. This yielded the title compound as a clear oil (0.091g, 78%).

¹H-NMR (400 MHz, CDCl₃) 1.29 (3H, t, J=7 Hz), 2.18 (3H, s), 4.28 (2H, q, J=7 Hz), 4.85 (2H, s), 6.17 (1H, d, J=3 Hz), 6.37 (1H, d, J=3 Hz), 6.67 (1H, d, J=9 Hz), 6.86-6.97 (2H, m), 7.11 (1H, dd, J=2 Hz, 9 Hz), 7.15 (1H, broad d, J=9 Hz), 7.31 (1H, t, J=8 Hz), 7.37 (1H, dd, J=2 Hz, 9 Hz), 7.50 (1H, d, J=2 Hz), 7.12 (1H, t, J=1 Hz), 7.93 (1H, dt, J=0.5 Hz, 8 Hz).

LC/MS t=4.34 min [MH⁺] 532/534.

### b) 3-{2-[5-Trifluoromethyl-2-(2-chloro-4-fluoro-benzyloxy)-phenyl]-5-methyl-pyrrol-1-yl}-benzoic acid

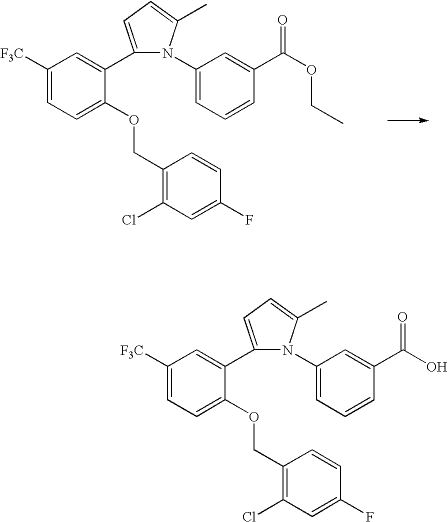

3-{2-[5-Trifluoromethyl-2-(2-chloro-4-fluoro-benzyloxy)-phenyl]-5-methyl-pyrrol-1-yl}-benzoic acid ethyl ester (0.091 g), 2M NaOH (1 ml) and EtOH (2 ml) were heated at 100° C. in a sealed vessel for 45 minutes. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound as a brown solid (0.085g, 99%).

¹H-NMR (400 MHz, CDCl₃) 2.18 (3H, s), 4.86 (2H, s), 6.17 (1H, d, J=3 Hz), 6.37 (1H, d, J=3 Hz), 6.71 (1H, d, J=9 Hz), 6.92 (1H, ddd, J=2 Hz, 8 Hz), 6.98-7.04 (1H, m), 7.09 (1H, dd, J=2 Hz, 8 Hz), 7.20 (1H, broad d, J=8 Hz), 7.33-7.39 (2H, m), 7.48 (1H, d, J=2 Hz), 7.77 (1H, t, J=1Hz), 7.98 (1H, dt, J=0.5 Hz, 8 Hz).

LC/MS t=4.10 min [MH⁺] 504/506.

### Example 77

### 3-(2-[5-Trifluoromethyl-2-(2,4-difluoro-benzyloxy)-phenyl]-5-methyl-pyrrol-1-yl}-benzoic acid

### a) -{2-[5-Trifluoromethyl-2-(2,4-difluoro-benzyloxy)-phenyl]-5-methyl-pyrrol-1-yl}-benzoic acid ethyl ester

3-{2-[5-Trifluoromethyl-2-(hydroxy)-phenyl]-5-methyl-pyrrol-1-yl}-benzoic acid ethyl ester (0.085g, 0.219 mmol), 2,4-difluorobenzyl bromide (0.043g, 0.329 mmol) and potassium carbonate (0.061g, 0.438 mmol) were heated in DMF (2 ml) at 65° C. in a nitrogen atmosphere for 3.5 hours. Upon cooling the mixture was diluted with EtOAc and washed with 2× water. The organic layer was extracted and the aqueous layer washed with 3×EtOAc. The combined organics were then washed with brine and concentrated in vacuo. The residue was purified by chromatography on silica gel eluting with 8% EtOAc/iso-hexane. This yielded the title compound as a clear oil (0.089g, 79%).

¹H-NMR (400 MHz, CDCl₃) 1.31 (3H, t, J=7 Hz), 2.18 (3H, s), 4.29 (2H, q, J=7 Hz), 4.84 (2H, s), 6.14 (1H, d, J=3 Hz), 6.34 (1H, d, J=3 Hz), 6.74 (1H, d, J=9 Hz), 6.77-6.83 (2H, m), 6.97-7.03 (1H, m) 7.13 (1H, broad d, J=8 Hz), 7.30 (1H, t, J=8 Hz), 7.37 (1H, dd, J=2 Hz, 9 Hz) 7.47 (1H, d, J=2 Hz), 7.70 (1H, t, J=1 Hz), 7.92 (1H, dt, J=0.5 Hz, 8 Hz).

LC/MS t=4.22 min [MH⁺] 516.

### b) 3-{2-[5-Trifluoromethyl-2-(2,4-difluoro-benzyloxy)-phenyl]-5-methyl-pyrrol-1-yl}-benzoic acid

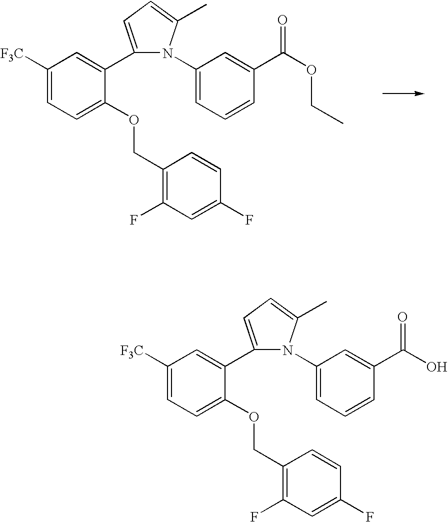

3-{2-[5-Trifluoromethyl-2-(2,4difluoro-benzyloxy)-phenyl]-5-methyl-pyrrol-1-yl}-benzoic acid ethyl ester (0.089g), 2M NaOH (1 ml) and EtOH (2 ml) were heated at 100° C. in a sealed vessel for 45 minutes. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound.

¹H-NMR (400 MHz, CDCl₃) 2.18 (3H, s), 4.85 (2H, s), 6.14 (1H, d, J=3 Hz), 6.36 (1H, d, J=3 Hz), 6.74-6.84 (3H, m), 7.03-7.08 (1H, m), 7.17-7.20 (1H, m) 7.33 (1H, t, J=8 Hz), 7.38 (1H, dd, J=2 Hz, 9 Hz) 7.45 (1H, d, J=2 Hz), 7.77 (1H, t, J=1 Hz), 7.98 (1H, dt, J=0.5 Hz, 8 Hz).

LC/MS t=3.97 min [MH⁺] 488.

### Example 78

### 3-{2-[5-Trifluoromethyl-2-(cyclohexylmethoxy)-phenyl-6-methyl-pyrrol-1-yl)--benzoic acid

### a) 3-{2-[5-Trifluoromethyl-2-(cyclohexylmethoxy)-phenyl]-5-methyl-pyrrol-1-yl]-benzoic acid ethyl ester

3-{2-[5-Trifluoromethyl-2-(hydroxy)-phenyl]-5-methyl-pyrrol-1-yl}-benzoic acid ethyl ester (0.085g, 0.219 mmol), cyclohexylmethylene bromide (0.042 ml, 0.329 mmol) and potassium carbonate (0.061g, 0.438 mmol) were heated in DMF (2 ml) at 65° C. in a nitrogen atmosphere for 3.5 hours. Upon cooling the mixture was diluted with EtOAc and washed with 2× water. The organic layer was extracted and the aqueous layer washed with 3×EtOAc. The combined organics were then washed with brine and concentrated in vacuo. The residue was purified by chromatography on silica gel eluting with 8% EtOAc/iso-hexane. This yielded the title compound as a clear oil (0.047g, 44%).

¹H-NMR (400 MHz, CDCl₃) 0.80-0.96 (2H+ excess, m), 1.10-1.31 (3H+ excess, m), 1.36 (3H, t, J=7 Hz), 1.65-1.73 (6H, m), 2.20 (3H, s), 3.51 (2H, d, J=7 Hz), 4.34 (2H, q, J=7 Hz), 6.13 (1H, d, J=3 Hz), 6.34 (1H, d, J=3 Hz), 6.73 (1H, d, J=9 Hz), 7.18 (1H, d, J=9 Hz), 7.30-7.37 (3H, m), 7.80 (1H, t, J=1 Hz), 7.92 (1H, dt, J=0.5 Hz, 8 Hz).

LC/MS t=4.50 min [MH⁺] 486.

### b) 3-{2-[5-Trifluoromethyl-2-(cyclohexylmethoxy)-phenyl]-5-methyl-pyrrol-1-yl}-benzoic acid

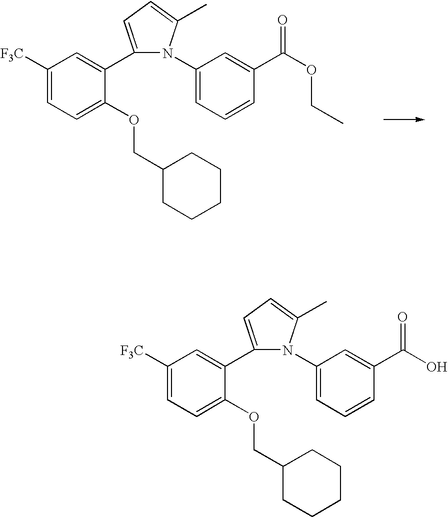

3-{2-[5-Trifluoromethyl-2-(cyclohexylmethoxy)-phenyl]-5-methyl-pyrrol-1-yl}-benzoic acid ethyl ester (0.047g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound.

¹H-NMR (400 MHz, CDCl₃) 0.85-0.96 (2H+ excess, m), 1.10-1.31 (3H+ excess, m) 1.63 -1.75 (6H, m), 2.20 (3H, s), 3.52 (2H, d, J=6 Hz), 6.14 (1H, d, J=3 Hz), 6.35 (1H, d, J=3 Hz), 6.75 (1H, d, J=9 Hz), 7.20-7.24 (1H, m), 7.30-7.39 (3H, m), 7.92 (1H, t, J=1 Hz), 7.98 (1H, dt, J=0.5 Hz, 8 Hz).

LC/MS t=4.28 min [MH⁺] 458.

### Example 79

### 3-{2-[5-Trifluoromethyl-2-(4-methoxy-benzyloxy)-phenyl]-5-methyl-pyrrol-1-yl}-benzoic acid

### a) 5-Trifluoromethyl-2-(4-methoxy-benzyloxy)-benzaldehyde

2-Hydroxy-5-trifluoromethyl-benzaldehyde (prepared via the procedure of Schäfer, Synthesis 2001, 15, 2259-2262) (0.25g, 1.32 mmol), 4-methoxybenzyl chloride (0.269 ml, 1.98 mmol) and potassium carbonate (0.363g, 2.63 mmol) were heated in DMF (7.5 ml) at 60° C. in a nitrogen atmosphere for 2 hour. Upon cooling the reaction mixture was diluted with EtOAc and washed with water. The organic layer was extracted and the aqueous layer washed with 3×EtOAc. The combined organic extracts were then washed with brine and dried over MgSO₄, filtered and concentrated in vacuo. To yield the title compound as a yellow oil which was carried through without further purification.

¹H-NMR (400 MHz, CDCl₃) 3.81 (3H, s) 5.19 (2H, s), 6.94 (2H, d, J=8 Hz), 7.18 (1H, d, J=9 Hz), 7.37 (2H, d, J=8 Hz) 7.78 (1H, d, J=9 Hz), 8.12 (1H, s) 10.55 (1H, s).

LC/MS t=3.58 min.

### b) 1-[5-Trifluoromethyl-2-(4-methoxy-benzyloxy)-phenyl]-pentane-1,4-dione

2-(4-Methoxy-benzyloxy)-5-trifluoromethyl-benzaldehyde (0.42g, 60%, 0.81 mmol), triethylamine (0.337 ml, 2.42 mmol), methyl vinyl ketone (0.068 ml, 0.82 mmol) and 3-ethyl-5-(2-hydroxyethyl)-4-ethylthiazolium bromide (0.061g, 0.24 mmol) were refluxed in EtOH (2 ml) under a nitrogen atmosphere for 22 hours. Upon cooling the reaction mixture was diluted with EtOAc and washed with saturated NH₄Cl. The organic layer was extracted and the aqueous layer washed with 3×EtOAc. The combined organic extracts were then washed with saturated NaHCO₃ and brine and then dried over MgSO₄, filtered and concentrated in vacuo. The residue was purified by chromatography, using Biotage^{□} 40M eluting with 15% EtOAc/iso-hexane. This yielded the title compound as an impure yellow oil (0.180g, 50% pure).

¹H-NMR (400 MHz, CDCl₃) 2.18 (3H, s), 2.77 (2H, t, J=6 Hz), 3.22 (2H, t, J=6 Hz), 3.73 (3H, s), 5.15 (2H, s), 6.91 (2H, d, J=9 Hz), 7.11 (1H, d, J=9 Hz), 7.37 (2H, d, J=9 Hz), 7.67 (1H, t, J=8 Hz), 8.02 (1H, d, J=14 Hz)

LC/MS t=3.50 min.

### c) 3-{2-[5-Trifluoromethyl-2-(4-methoxy-benzyloxy)-phenyl]-5-methyl-pyrrol-1-yl}-benzoic acid ethyl ester

1-[5-Trifluoromethyl-2-(4-methoxy-benzyloxy)-phenyl]-pentane-1,4dione (0.180g, 50% pure, 0.24 mmol) and ethyl-3-aminobenzoate (0.042 ml, 0.28 mmol) were heated in a sealed vessel at 140° C. for 18 hours. Upon cooling the residue was purified by chromatography on silica gel eluting with 10% EtOAc/iso-hexane. This yielded the title compound as a yellow oil (0.03g, 25%).

¹H-NMR (400 MHz, CDCl₃) 1.29 (3H, t, J=7 Hz), 2.15 (3H, s), 3.80 (3H, s) 4.28 (2H, q, J=7 Hz), 4.74 (2H, s), 6.13 (1H, d, J=3 Hz), 6.35 (1H, d, J=3 Hz), 6.73 (1H, d, J=9 Hz), 6.83 (2H, d, J=9 Hz), 7.01 (2H, d, J=9 Hz), 7.12 (1H, d, J=8 Hz) 7.25-7.35 (2H, m), 7.46 (1H, d, J=2 Hz), 7.70 (1H, s), 7.91 (1H, d, J=8 Hz).

LC/MS t=4.17 [MH⁺] 510.

### d) 3-{2-[5-Trifluoromethyl-2-(4-methoxy-benzyloxy)-phenyl]-5-methyl-pyrrol-1-yl}-benzoic acid

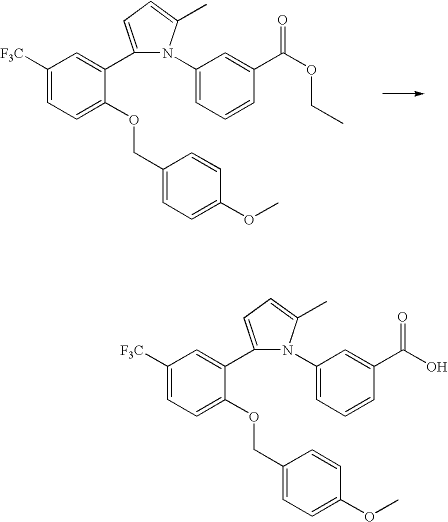

3-{2-[5-Trifluoromethyl-2-(4-methoxy-benzyloxy)-phenyl]-5-methyl-pyrrol-1-yl}-benzoic acid ethyl ester (0.030g), 2M NaOH (1 ml) and EtOH (2.5 ml) were heated at 100° C. in a sealed vessel for 30 minutes. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated in vacuo, to yield the title compound as a brown oil (0.019g, 67%)

¹H-NMR (400 MHz, CDCl₃) 2.16 (3H, s), 3.77 (3H, s), 4.75 (2H, s), 6.14 (1H, d, J=3 Hz), 6.35 (1H, d, J=3 Hz), 6.74 (1H, d, J=9 Hz), 6.83 (2H, d, J=9 Hz), 7.04 (2H, d, J=9 Hz), 7.14-7.18 (1H, m) 7.28-7.36 (2H, m), 7.45 (1H, d, J=2 Hz), 7.78 (1H, t, J=1 Hz), 7.96 (1H, d, J=8 Hz).

LC/MS t=3.89 min [MH⁻] 480.

### Example 80

### 3-[2-(2-Benzyloxy-phenyl)-5-methyl-pyrrol-1-yl]-N-(1-phenylsulfonyl)-benzamide

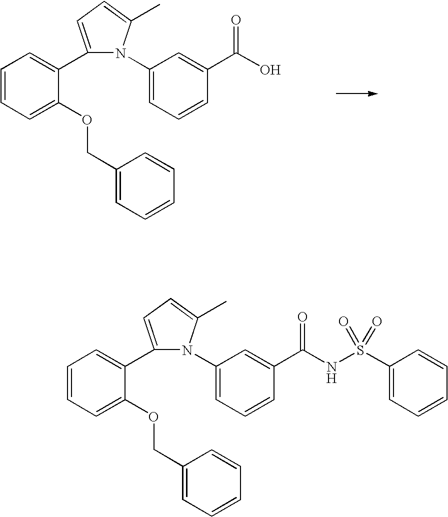

Benzenesulfonamide (31 mg, 0.20 mmol) was added to 3-[2-(2-benzyloxy-phenyl)-5-methyl-pyrrol-1-yl)--benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days.

Upon cooling, the reaction mixture was diluted with EtOAc, washed with 2M HCl, brine, dried (MgSO₄), filtered and concentrated. The residue was purified using MDAP to give the title compound (10 mg, 18%).

¹H-NMR (400 MHz, d₆-DMSO) 2.05 (3H, s), 4.81 (2H, s), 6.04 (1H, d, J=3 Hz), 6.17 (1H, d, J=3 Hz), 6.77-6.84 (2H, m), 7.02-7.31 (8H, m), 7.38 (1H, t, J=8 Hz), 7.55-7.73 (4H, m), 7.78 (1H, d, J=8 Hz), 7.95 (2H, d, J=8 Hz), 12.60 (1H, broad s).

LC/MS 1 t=4.15 min [MH⁺] 423.

### Example 81

### 3-{2-[2-(4-Chloro-benzyloxy)-phenyl]-5-methyl-pyrrol-1-yl}-N-(1-phenylsulfonyl)-benzamide

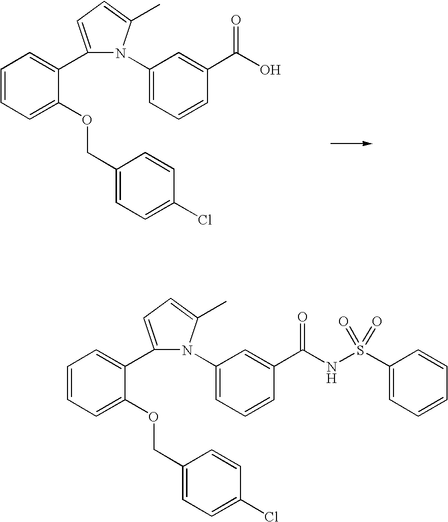

Benzenesulfonamide (31 mg, 0.20 mmol) was added to 3-{2-[2-(4-chloro-benzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days. Upon cooling, the reaction mixture was diluted with EtOAc, washed with 2M HCl, brine, dried (MgSO₄), filtered and concentrated. The residue was purified using MDAP to give the title compound (12 mg, 24%).

¹H-NMR (400 MHz, d₈-DMSO) 2.05 (3H, s), 4.81 (2H, s), 6.04 (1H, d, J=3.5 Hz), 6.16 (1H, d, J=3.5 Hz), 6.76-6.84 (2H, m), 7.03-7.16 (4H, m), 7.20 (1H, d, J=8 Hz), 7.31-7.41 (3H, m), 7.55-7.73 (4H, m), 7.79 (1H, d, J=8 Hz), 7.95 (2H, d, J=8 Hz), 12.60 (1H, broad s).

LC/MS t=4.34 min [MH⁺] 557/559.

### Example 82

### 3-{2-[2-(4-Fluoro-benzyloxy)-phenyl]-5-methyl-pyrrol-1-yl}-N-(1-phenylsulfonyl)-benzamide

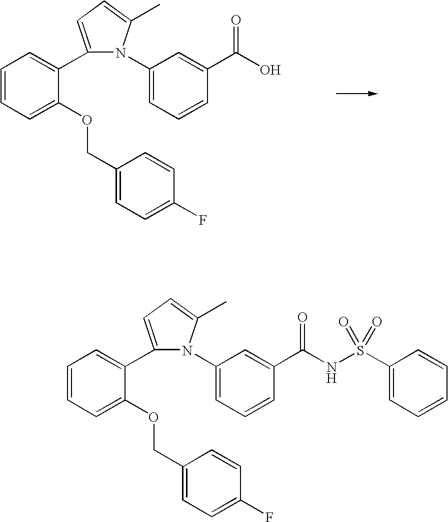

Benzenesulfonamide (31 mg, 0.20 mmol) was added to 3-{2-[2-(4-fluoro-benzyloxy)-phenyl]-5-methyl-pyrrol-1-yl}-benzoic acid (40 mg, 0.10 mmol), carbonyl diimidazole (33 mg, 0.20 mmol) and diisopropylethylamine (0.035 ml, 0.20 mmol) in dichloromethane (2 ml) and heated at reflux for 2 days. Upon cooling, the reaction mixture was diluted with dichloromethane, washed with 2M HCl, brine, dried (MgSO₄), filtered and concentrated. The residue was purified using MDAP to give the title compound (19 mg, 36%).

¹H-NMR (400 MHz, d₆-DMSO) 1.99 (3H, s), 4.86 (2H, s), 5.99 (1H, d, J=3 Hz), 6.13 (1H, d, J=3 Hz), 6.76 (1H, t, J=8 Hz), 6.85 (1H, d, J=9 Hz), 6.95-6.99 (2H, m), 7.06-7.23 (4H, m), 7.26-7.39 (5H, m), 7.61 (1H, broad s), 7.76-7.82 (3H, m).

LC/MS t=4.18 min [MH⁺] 541.

### Example 83

### 3-{2-[2-(2-Chloro-4-fluoro-benzyloxy)-phenyl]-5-methyl-pyrrol-1-yl}-N-(1-phenylsulfonyl)-benzamide

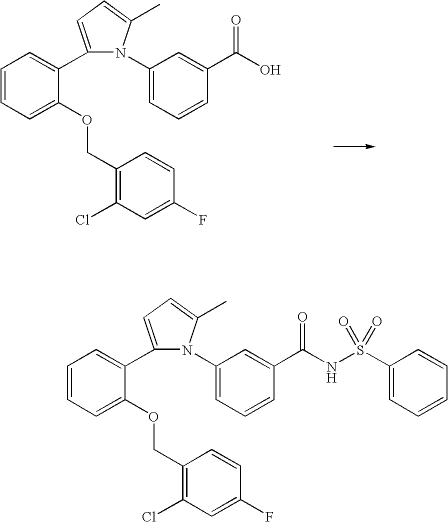

Benzenesulfonamide (31 mg, 0.20 mmol) was added to 3-{2-[2-(2-chloro-4-fluorobenzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days. Upon cooling, the reaction mixture was diluted with EtOAc, washed with 2M HCl, brine, dried (MgSO₄), filtered and concentrated. The residue was purified using MDAP to give the title compound (8 mg, 15%).

¹H-NMR (400 MHz, d₆-DMSO) 2.05 (3H, s), 4.82 (2H, s), 6.04 (1H, d, J=3.5 Hz), 6.18 (1H, d, J=3.5 Hz), 6.79-6.87 (2H, m), 7.04-7.16 (4H, m), 7.20 (1H, d, J=8 Hz), 7.35-7.44 (2H, m), 7.54 (1H, broad s), 7.62 (2H, broad t, J=8 Hz), 7.71 (1H, m), 7.79 (1H, d, J=8 Hz), 7.95 (2H, d, J=8 Hz), 12.55 (1H, broad s).

LC/MS t=4.37 min [MH⁺] 575/577.

### Example 84

### 3-{2-[2-(2,4-Difluoro-benzyloxy)-phenyl]-5-methyl-pyrrol-1-yl}-N-(1-phenylsulfonyl)-benzamide

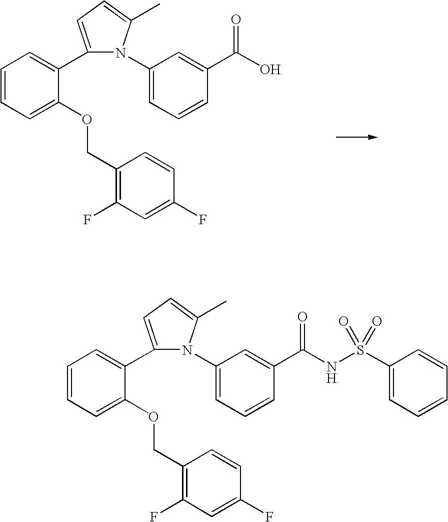

Benzenesulfonamide (31 mg, 0.20 mmol) was added to 3-{2-[2-(2,4-difluorobenzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days. Upon cooling, the reaction mixture was diluted with EtOAc, washed with 2M HCl, brine, dried (MgSO₄), filtered and concentrated. The residue was purified using MDAP to give the title compound (1 0 mg, 20%).

¹H-NMR (400 MHz, d₆-DMSO) 2.03 (3H, s), 4.81 (2H, s), 6.02 (1H, d, J=3.5 Hz), 6.14 (1H, d, J=3.5 Hz), 6.82 (1H, t, J=8 Hz), 6.89 (1H, d, J=8 Hz), 6.96-7.05 (2H, m), 7.10-7.22 (4H, m), 7.37 (1H, t, J=8 Hz), 7.52 (1H, broad s), 7.58-7.72 (3H, m), 7.79 (1H, d, J=8 Hz), 7.95 (2H, d, J=8 Hz), 12.60 (1H, broad s).

LC/MS t=4.19 min [MH⁺] 559.

### Example 85

### 3-[2-(5-Chloro-2-benzyloxy-phenyl)-5-methyl-pyrrol-1-yl]-N-(1-phenylsulfonyl)-benzamide

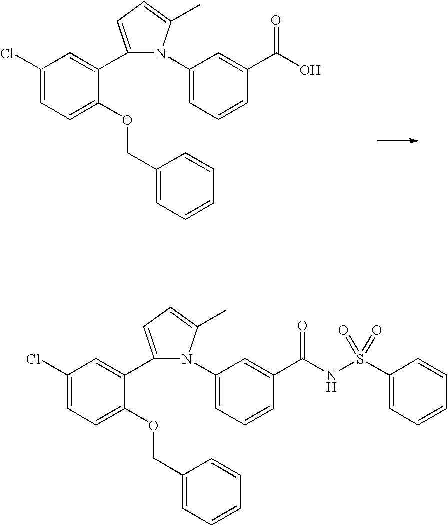

3-{2-[5-Chloro-2-(benzyloxy)-phenyl]-5-methyl-pyrrol-1-yl}-benzoic acid (0.02g, 0.048 mmol), carbonyldiimidazole (0.016g, 0.096 mmol), diisopropylethylamine (0.017 ml, 0.096 mmol), benzenesulfonamide (0.01 5g, 0.096 mmol) and DCM (4 ml) were stirred under a nitrogen atmosphere at reflux for 5 days. The reaction was diluted with DCM and washed with 2M HCl. The organics were separated and the aqueous washed with 3×DCM, the combined organics were then dried over MgSO₄, filtered and the solvent removed in vacuo to yield a white solid which was purified on mass-directed auto prep to yield the title compound as a white solid (0.012g, 44%).

¹H-NMR (400 MHz, CDCl₃) 2.11 (3H, s), 4.71 (2H, s), 6.13 (1H, d, J=3 Hz), 6.31 (1H, d, J=3 Hz), 6.62 (1H, d, J=9 Hz), 7.02-7.05 (2H, m), 7.08 (1H, dd, J=3 Hz, 9 Hz), 7.15 (1H, d, J=9 Hz), 7.20 (1H, d, J=3 Hz), 7.27-7.31 (5H, m), 7.52-7.67 (4H, m), 8.09 (2H, d, J=8 Hz).

LC/MS t=4.35 min [MH⁺] 557.

### Example 86

### 3-{2-[5-Chloro-2-(4-fluoro-benzyloxy)-phenyl]-5-methyl-pyrrol-1-yl}-N-(1-phenylsulfonyl)-benzamide

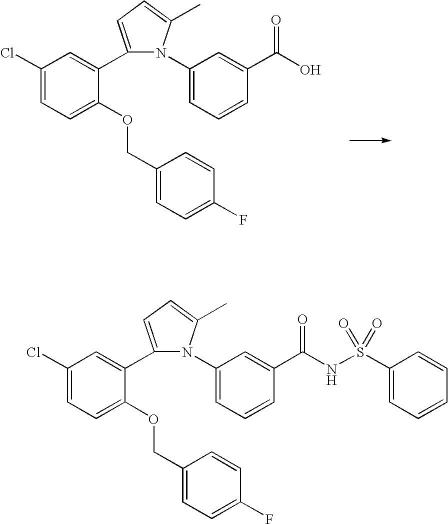

3-{2-[5-Chloro-2-(4-fluoro-benzyloxy)-phenyl]-5-methyl-pyrrol-1-yl}-benzoic acid (0.02g, 0.046 mmol), carbonyldiimidazole (0.015g, 0.092 mmol), diisopropylethylamine (0.016 ml, 0.092 mmol), benzenesulfonamide (0.014g, 0.092 mmol) and DCM (4 ml) were stirred under a nitrogen atmosphere at reflux for 5 days. The reaction was diluted with DCM and washed with 2M HCl. The organics were separated and the aqueous washed with 3×DCM, the combined organics were then dried over MgSO₄, filtered and the solvent removed in vacuo to yield a white solid which was purified on mass-directed auto prep to yield the title compound as a white solid (0.011 g, 42%)

¹H-NMR (400 MHz, CDCl₃) 2.10 (3H, s), 4.69 (2H, s), 6.12 (1H, d, J=3 Hz), 6.29 (1H, d, J=3 Hz), 6.61 (1H, d, J=9 Hz), 6.94-6.99 (2H, m), 7.02-7.05 (2H, m), 7.07 (1H, dd, J=3 Hz, 9 Hz), 7.15-7.17 (1H, m), 7.18 (1H, d, J=3 Hz), 7.30 (1H, t, J=8 Hz), 7.37 (1H, broad s), 7.53-7.68 (4H, m), 8.10 (2H, d, J=8 Hz).

LC/MS t=4.36 min [MH⁺] 575.

### Example 87

### 3-{2-[5-Chloro-2-(2,4-difluoro-benzyloxy)-phenyl]-5-methyl-pyrrol-1-yl}-N-(1-phenylsulfonyl)-benzamide

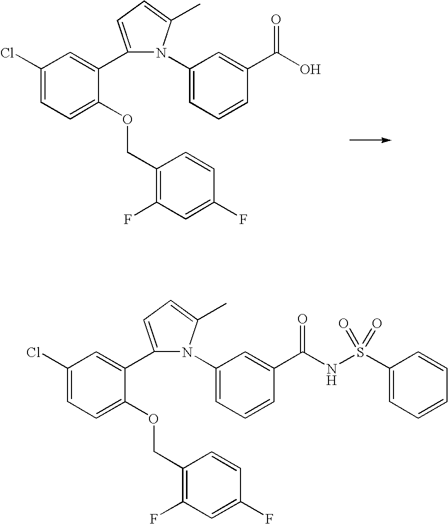

3-{2-[5-Chloro-2-(2,4-difluoro-benzyloxy)-phenyl]-5-methyl-pyrrol-1-yl}-benzoic acid (0.039, 0.066 mmol), carbonyldiimidazole (0.012g, 0.073 mmol), diisopropylethylamine (0.013 ml, 0.073 mmol), benzenesulfonamide (0.012g, 0.073 mmol) and DCM (5 ml) were stirred under a nitrogen atmosphere at reflux for 2 days. The reaction was diluted with DCM and washed with 2M HCl. The organics were separated and the aqueous washed with 3×DCM, the combined organics were then dried over MgSO₄, filtered and the solvent removed in vacuo to yield a white solid which was purified on mass-directed auto prep to yield the title compound as a white solid (0.019g, 49%).

¹H-NMR (400 MHz, CDCl₃) 2.09 (3H, s), 4.70 (2H, s), 6.08 (1H, d, J=3 Hz), 6.26 (1H, d, J=3 Hz), 6.62 (1H, d, J=9 Hz), 6.68-6.79 (2H, m), 6.95-7.02 (1H, m), 7.16 (1H, dd, J=3 Hz, 9 Hz), 7.12-7.17 (2H, m), 7.25-7.31 (1H+excess, m), 7.46 (1H, broad s), 7.52-7.57 (2H, m), 7.63-7.68 (2H, m), 8.10 (2H, d, J=8 Hz).

LC/MS t=4.40 min [MH+] 593/595.

### Example 88

### 3-[2-(2-Benzyloxy-phenyl}-5-methyl-pyrrol-1-yl]-N-(3,5-dimethyl-isoxazole-4-sulfonyl)-benzamide

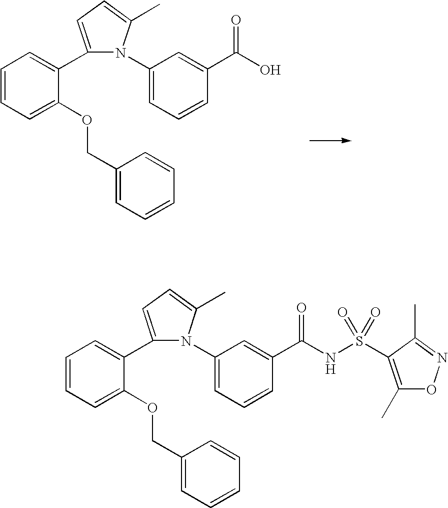

3,5-Dimethyl-isoxazole-4-sulfonamide (35 mg, 0.20 mmol) was added to 3-{-[2-(benzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days. Upon cooling, the reaction mixture was diluted with EtOAc, washed with 2M HCl, brine, dried (MgSO₄), filtered and concentrated. The residue was purified using MDAP to give the title compound (16 mg, 30%).

¹H-NMR (400 MHz, d₆-DMSO) 2.06 (3H, s), 2.31 (3H, s), 2.63 (3H, s), 4.85 (2H, s), 6.04 (1H, d, J=3 Hz), 6.18 (1H, d, J=3 Hz), 6.76-6.84 (2H, m), 7.01-7.34 (8H, m), 7.38 (1H, t, J=8 Hz), 7.62 (1H, broad s), 7.81 (1H, d, J=8 Hz), 12.90 (1H, broad s).

LC/MS t=4.31 min [MH⁺] 542.

### Example 89

### 3-{2-[2-(4-Chloro-benzyloxy)-phenyl]-5-methyl-pyrrol-1-yl}-N-(3,5-dimethyl-isoxazole-4-sulfonyl)-benzamide

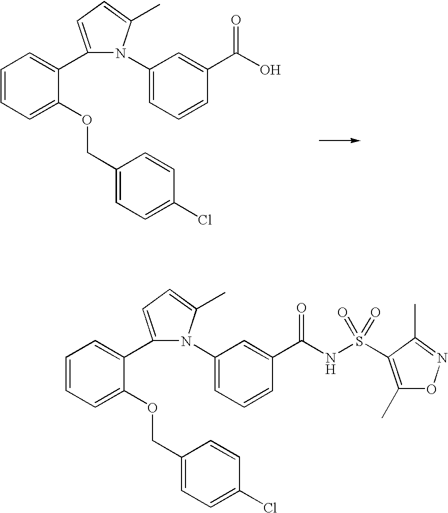

3,5-Dimethyl-isoxazole-4-sulfonamide (35 mg, 0.20 mmol) was added to 3-{2-[2-(4-chloro-benzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at 75° C. for 4 days. Upon cooling, the reaction mixture was diluted with EtOAc, washed with 2M HCl, brine, dried (MgSO₄), filtered and concentrated. The residue was purified using MDAP to give the title compound (10 mg, 19%).

¹H-NMR (400 MHz, d₆-DMSO) F7465 2.06 (3H, s), 2.30 (3H, s), 2.63 (3H, s), 4.84 (2H, s), 6.04 (1H, d, J=3.5 Hz), 6.17 (1H, d, J=3.5 Hz), 6.56 (1H, broad s), 6.82 (2H, t, J=8 Hz), 7.03-7.23 (5H, m), 7.32-7.41 (3H, m), 7.60 (1H, broad s), 7.82 (1H, d, J=8 Hz).

LC/MS CF107228-1 t=4.50 [MH⁺] 576/578.

### Example 90

### 3-{2-[2-(4-Fluoro-benzyloxy)-phenyl]-5-methyl-pyrrol-1-yl}-N-(3,5-dimethyl-isoxazole-4-sulfonyl)-benzamide

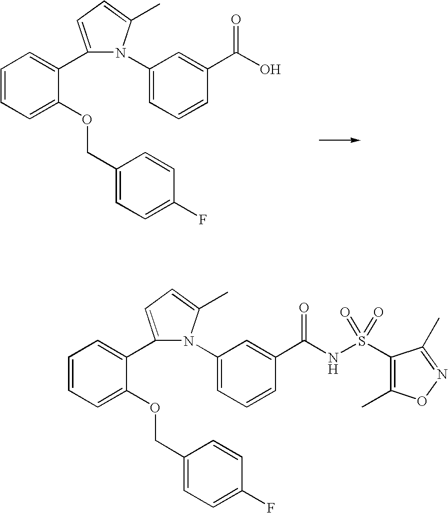

3,5-Dimethyl-isoxazole-4-sulfonamide (35 mg, 0.20 mmol) was added to 3-{2-[2-(4-fluoro-benzyloxy)-phenyl]-5-methyl-pyrrol-1-yl}-benzoic acid (40 mg, 0.10 mmol), carbonyl diimidazole (33 mg, 0.20 mmol) and diisopropylethylamine (0.035 ml, 0.20 mmol) in dichloromethane (2 ml) and heated at reflux for 2 days. Upon cooling, the reaction mixture was diluted with dichloromethane, washed with 2M HCl, brine, dried (MgSO₄), filtered and concentrated. The residue was purified using MDAP to give the title compound (12 mg, 21%).

¹H-NMR (400 MHz, d₆-DMSO) 2.05 (3H, s), 2.30 (3H, s), 2.62 (3H, s), 4.83 (2H, s), 6.03 (1H, d, J=3.5 Hz), 6.16 (1H, d, J=3.5 Hz), 6.53 (1H, broad s), 6.77-6.85 (2H, m), 7.04 (1H, d, J=7.5 Hz), 7.07-7.25 (6H, m), 7.32-7.41 (1H, m), 7.60 (1H, broad s), 7.81 (1H, d, J=8 Hz).

LC/MS t=4.34 min [MH⁺] 560.

### Example 91 1

### 3-{2-[2-(2-Chloro-4-fluoro-benzyloxy)-phenyl]-5-methyl-pyrrol-1-yl}-N-(3,5-dimethyl-isoxazole-4-sulfonyl)-benzamide

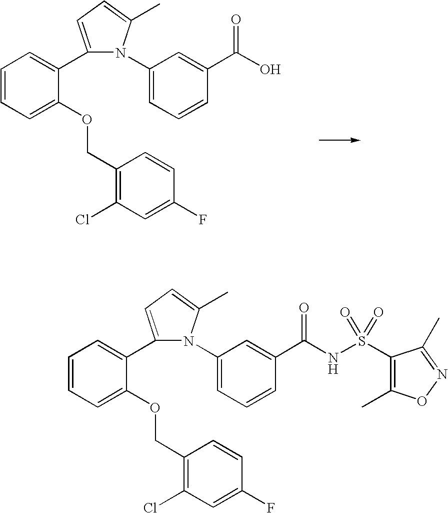

3,5-Dimethyl-isoxazole-4-sulfonamide (35 mg, 0.20 mmol) was added to 3-{2-[2-(2-chloro-4-fluorobenzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days. Upon cooling, the reaction mixture was diluted with EtOAc, washed with 2M HCl, brine, dried (MgSO₄), filtered and concentrated. The residue was purified using MDAP to give the title compound (7 mg, 13%).

¹H-NMR (400 MHz, d₆-DMSO) 2.06 (3H, s), 2.30 (3H, s), 2.62 (3H, s), 4.83 (2H, s), 6.04 (1H, d, J=3.5 Hz), 6.19 (1H, d, J=3.5 Hz), 6.82 (1H, t, J=8 Hz), 6.90 (1H, d, J=8 Hz), 6.99-7.06 (2H, m), 7.10-7.25 (4H, m), 7.33-7.40 (1H, m), 7.56 (1H, broad s), 7.81 (1H, d, J=8 Hz), 12.80 (1H, broad s).

LC/MS t=4.55 min [MH⁺] 594/596.

### Example 92

### 3-{2-[2-(2,4-Difluoro-benzyloxy)-phenyl]-5-methyl-pyrrol-1-yl}-N-(3,5-dimethyl-isoxazole-4-sulfonyl)-benzamide

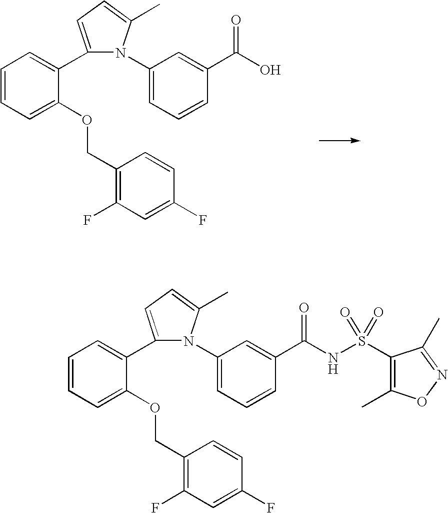

3,5-Dimethyl-isoxazole-4-sulfonamide (35 mg, 0.20 mmol) was added to 3-{2-[2-(2,4-difluorobenzyloxy)-phenyl]-5-methyl-pyrrol-1-yl}-benzoic acid (40 mg, 0.09 mmol), carbonyl diimidazole (33 mg, 0.20 mmol) and diisopropylethylamine (0.035 ml, 0.20 mmol) in THF (2 ml) and heated at reflux for 4 days. Upon cooling, the reaction mixture was diluted with EtOAc, washed with 2M HCl, brine, dried (MgSO₄), filtered and concentrated. The residue was purified using MDAP to give the title compound (12 mg, 23%).

¹H-NMR (400 MHz, d₆-DMSO) 2.04 (3H, s), 2.32 (3H, s), 2.63 (3H, s), 4.84 (2H, s), 6.02 (1H, d, J=3.5 Hz), 6.15 (1H, d, J=3.5 Hz), 6.52 (1H, broad s), 6.81-6.87 (2H, m), 7.04-7.22 (5H, m), 7.33-7.44 (2H, m), 7.57 (1H, broad s), 7.82 (1H, d, J=8 Hz).

LC/MS t=4.37 min [MH⁺] 578.

### Example 93

### 3-{2-[2-(4-Fluoro-benzyloxy)-phenyl]-5-methyl-pyrrol-1-yl}-N-(1-phenyl-methanoyl)-benzenesulfonamide

### a) 2-(4-Fluoro-benzyloxy)-benzaldehyde

Procedure as for 2-benzyloxy-5-chloro-benzaldehyde to give the title compound.

LCMS t=3.30 min

### b) 1-[2-(4-Fluoro-benzyloxy)-phenyl]-pentane-1,4-dione

Procedure as for 1-[5-chloro-2-(benzyloxy)-phenyl]-pentane-1,4-dione to give the title compound.

LCMS t=3.29 min.

### c) 3-{2-[2-(4-Fluoro-benzyloxy)-phenyl]-5-methyl-pyrrol-1-yl}-benzene-sulfonamide

1-[2-(4-Fluoro-benzyloxy)-phenyl]-pentane-1,4-dione (1.5g, 5 mmol), 3-aminobenzenesulfonamide (1.03g, 6 mmol) (ex. Maybridge), para-toluenesulfonic acid (0.19g, 1 mmol) and toluene (30 ml) were stirred at reflux in a nitrogen atmosphere for 19 hours. The solvent was removed in vacuo and the residue taken up in EtOAc and the solution washed with NaHCO₃. The organics were extracted and the aqueous washed with 2×EtOAc. The combined organics were washed with brine, dried over MgSO₄, filtered and concentrated in vacuo. The residue was purified by chromatography using Biotage^{□} 40M was purified by chromatography on silica gel eluting with a gradient of 15%-30% EtOAc/iso-hexane. This yielded the title compound as a pale yellow solid (1.53g, 70%).

¹H-NMR (400 MHz, CDCl₃) 2.20 (3H, s), 4.34 (2H, broad s), 4.72 (2H, s), 6.15 (1H, d, J=3 Hz), 6.30 (1H, d, J=3 Hz), 6.65 (1H, d, J=8 Hz), 6.92 (1H, t, J=7 Hz), 6.97-7.08 (4H, m), 7.11-7.18 (2H, m), 7.27 (1H, m), 7.34 (1H, t, J=7 Hz), 7.49 (1H, t, J=1 Hz), 7.72 (1H, m).

LC/MS t=3.62 min [MH⁺] 437.

### d) 3-{2-[2-(4-Fluoro-benzyloxy)-phenyl]-5-methyl-pyrrol-1-yl}-N-(1-phenyl-methanoyl)-benzenesulfonamide

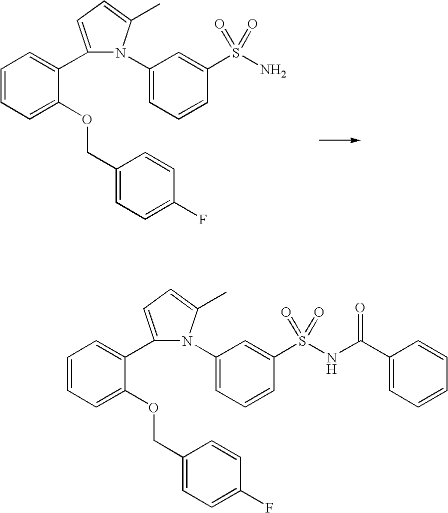

Benzoyl chloride (0.030 ml, 0.25 mmol) was added to 3-{2-[2-(4-fluoro-benzyloxy)-phenyl]-5-methyl-pyrrol-1-yl}-benzenesulfonamide (93 mg, 0.21 mmol), DMAP (26 mg, 0.21 mmol) and triethylamine (0.035 ml, 0.25 mmol) in dichloromethane (1 ml) and stirred at room temperature for 16 hours. The reaction mixture was diluted with dichloromethane, washed with 2M aqueous citric acid, brine, dried (MgSO₄), filtered and concentrated. The residue was purified by chromatography on silica gel with isohexane/EtOAc (15-25%) as eluant, to give the title compound (116 mg, 100%).

¹H-NMR (400 MHz, CDCl₃) 2.20 (3H, s), 4.59 (2H, s), 6.18 (1H, d, J=3.5 Hz), 6.28 (1H, d, J=3.5 Hz), 6.35 (1H, d, J=8 Hz), 6.69 (1H, ddd, J=2, 9 Hz), 6.76 (1H, broad t, J=8 Hz), 6.92-7.02 (4H, m), 7.18-7.23 (2H, m), 7.37 (1H, t, J=8 Hz), 7.48 (2H, t, J=8 Hz), 7.57-7.69 (4H, m), 8.04 (1H, broad d, J=8 Hz), 8.50 (1H, broad s).

LC/MS t=4.11 min [MH⁺] 541.

### Example 94

### 3-{2-[2-(2,4-Difluoro-benzyloxy)-phenyl]-5-methyl-pyrrol-1-yl]-N-(1-phenyl-methanoyl)-benzenesulfonamide

### a) 2-(2,4-Difluoro-benzyloxy)-benzaldehyde

Procedure as for 2-benzyloxy-5-chloro-benzaldehyde to give the title compound.

LCMS t=3.36 min

### b) 1-[2-(2,4-Difluoro-benzyloxy)-phenyl]-pentane-1,4-dione

Procedure as for 1-[5-chloro-2-(benzyloxy)-phenyl]-pentane-1,4-dione to give the title compound.

LCMS t=3.32 min

### c) 3-{2-[2-(2,4-Difluoro-benzyloxy)-phenyl]-5-methyl-pyrrol-1-yl}-benzene-sulfonamide

1-[2-(2,4-Difluoro-benzyloxy)-phenyl]-pentane-1,4-dione (1 g, 2.20 mmol), 3-aminonobenzenesulfonamide (0.65g, 2.64 mmol), para-toluenesulfonic acid (cat.) and toluene (50 ml) were stirred at reflux in a nitrogen atmosphere for 34 hours. The solvent was removed in vacuo and the residue taken up in EtOAc and the solution washed with 2N HCl and NaHCO₃, dried over MgSO₄, filtered and concentrated in vacuo. The residue was purified by chromatography on silica gel eluting with a gradient of 20% EtOAc/iso-hexane. This yielded the title compound as a pale yellow solid (1.75g, 100%).

¹H-NMR (400 MHz, CDCl₃) 2.20 (3H, s), 4.61 (2H, broad s), 4.77 (2H, s), 6.12 (1H, d, J=3 Hz), 6.29 (1H, d, J=3 Hz), 6.69 (1H, d, J=8 Hz), 6.75-6.84 (2H, m), 6.91 (1H, t, J=7 Hz), 6.99-7.06 (1H, m), 7.12-7.25 (3H, m), 7.34 (1H, t, J=7 Hz), 7.51 (1H, t, J=1 Hz), 7.72 (1H, m).

LC/MS t=3.62 min [MH⁺] 455.

### d) 3-{2-[2-(2,4Difluoro-benzyloxy)-phenyl]-5-methyl-pyrrol-1-yl}-N-(1-phenyl-methanoyl)-benzenesulfonamide

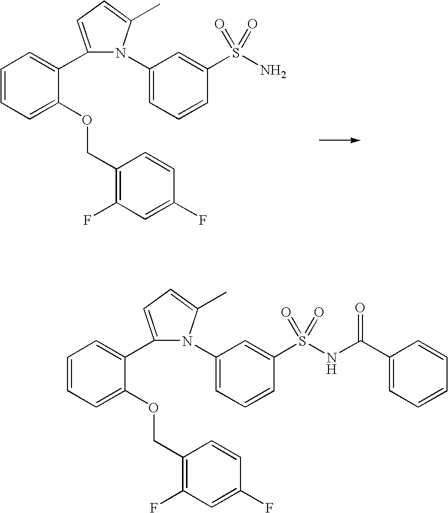

Benzoyl chloride (0.030 ml, 0.25 mmol) was added to 3-{2-[2-(2,4-difluoro-benzyloxy)-phenyl]-5-methyl-pyrrol-1-yl}-benzenesulfonamide (97 mg, 0.21 mmol), DMAP (26 mg, 0.21 mmol) and triethylamine (0.035 ml, 0.25 mmol) in dichloromethane (1 ml) and stirred at room temperature for 16 hours. The reaction mixture was diluted with dichloromethane, washed with 2M aqueous citric acid, brine, dried (MgSO₄), filtered and concentrated. The residue was purified by chromatography on silica gel with isohexane/EtOAc (15-25%) as eluant, to give the title compound (99 mg, 81%).

¹H-NMR (400 MHz, CDCl₃) 2.21 (3H, s), 4.70 (2H, s), 6.14 (1H, d, J=3.5 Hz), 6.27 (1H, d, J=3.5 Hz), 6.39-6.45 (1H, m), 6.69-6.82 (4H, m), 6.89-6.96 (1H, m), 7.15-7.20 (1H, m), 7.24-7.28 (1H, m), 7.41 (2H, t, J=8 Hz), 7.47 (1H, t, J=8 Hz), 7.61 (1H, broad t, J=8 Hz), 7.68 (2H, broad d, J=8 Hz), 7.74 (1H, t, J=1.5 Hz), 8.04 (1H, broad d, J=8 Hz), 8.50 (1H, broad s).

LC/MS t=4.13 min [MH⁺] 559.

### Example 95

### 3-{2-[5-Chloro-2-(benzyloxy)-phenyl]-5-methyl-pyrrol-1-yl}-N-(1-phenyl-methanoyl)-benzenesulfonamide

### a) 3-{2-[5-Chloro-2-(benzyloxy)-phenyl]-5-methyl-pyrrol-1-yl}-benzene-sulfonamide

1-[5-chloro-2-(benzyloxy)-phenyl]-pentane-1,4-dione (1g, 3.2 mmol), 3-amino-benzenesulfonamide (654 mg, 3.8 mmol) and para-toluenesulfonic acid (120 mg, 0.63 mmol) were heated at 1 10° C. in toluene (32 ml) for 16 hours. Upon cooling, the mixture was concentrated. The residue was purified by chromatography on silica gel with isohexane/EtOAc (25%) as eluant, to give the title compound (1.52g, 100%).

¹H-NMR (400 MHz, CDCl₃) 2.19 (3H, s), 4.25 (2H, s), 4.75 (2H, s), 6.16 (1H, d, J=3.5 Hz), 6.33 (1H, d, J=9 Hz), 6.58 (1H, d, J=9 Hz), 7.01 (2H, broad d, J=8 Hz), 7.08 (1H, dd, J=3, 9 Hz), 7.13 (1H, broad d, J=8 Hz), 7.25-7.43 (5H, m), 7.57 (1H, broad s), 7.74 (1H, broad d, J=8 Hz).

LC/MS t=3.73 min [MH⁺] 453/455.

### b) 3-{2-[5-Chloro-2-(benzyloxy)-phenyl]-5ethyl-pyrrol-1-yl}-N-(1-phenyl-methanoyl)-benzenesulfonamide

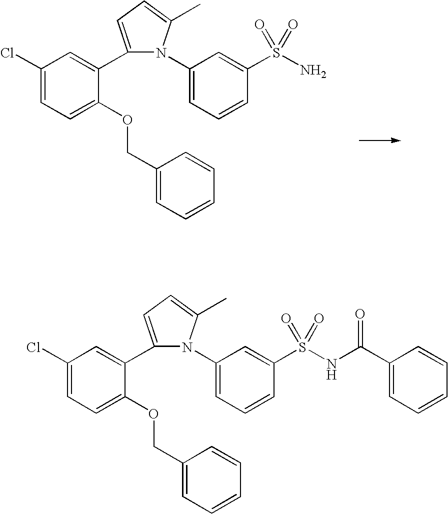

Benzoyl chloride (0.030 ml, 0.25 mmol) was added to 3-[2-(5-chloro-2-benzyloxy-phenyl}-5-methyl-pyrrol-1-yl]-benzenesulfonamide (96 mg, 0.21 mmol), DMAP (26 mg, 0.21 mmol) and triethylamine (0.035 ml, 0.25 mmol) in dichloromethane (1 ml) and stirred at room temperature for 16 hours. The reaction mixture was diluted with dichloromethane, washed with 2M aqueous citric acid, brine, dried (MgSO₄), filtered and concentrated. The residue was purified by chromatography on silica gel with isohexane/EtOAc (15-25%) as eluant, to give the title compound (95 mg, 80%).

¹H-NMR (400 MHz, CDCl₃) 2.20 (3H, s), 4.60 (2H, s), 6.14 (1H, d, J=3.5 Hz), 6.25 (1H, d, J=9 Hz), 6.29 (1H, d, J=3.5 Hz), 6.53 (1H, dd, J=3, 9 Hz), 6.97 (2H, broad d, J=8 Hz), 7.18-7.31 (5H, m), 7.39 (1H, t, J=8 Hz), 7.47 (1H, broad t, J=8 Hz), 7.58-7.67 (3H, m), 7.70 (1H, broad t, J=1.5 Hz), 8.07 (1H, broad d, J=8 Hz), 8.44 (1H, broad s).

LC/MS t=4.27 min [MH⁺] 557/559.

### Example 96

### 3-{2-[5-Chloro-2-(benzyloxy)-phenyl]-5-methyl-pyrrol-1-yl}-N-acetyl-benzenesulfonamide

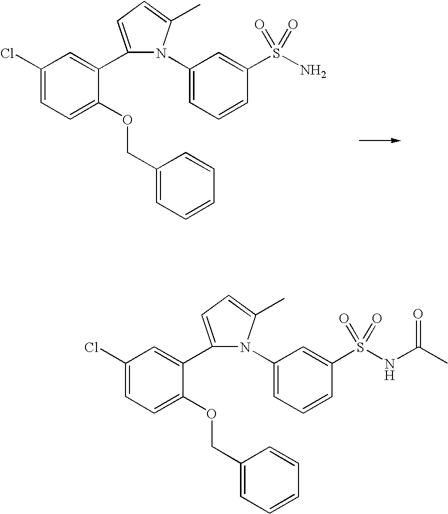

3-{2-[5-Chloro-2-(benzyloxy)-phenyl]-5-methyl-pyrrol-1-yl}-benzenesulfonamide (0.20g, 0.442 mmol), acetic anhydride (0.046 ml, 0.486 mmol), pyridine (0.072 ml, 0.884 mmol), dimethylaminopyridine (cat.) and DCM (2.5 ml) were stirred at reflux for 30 minutes. The reaction was washed with 2M HCl and the solvent removed in vacuo to yield the title compound as a brown oil (0.240g).

¹H-NMR (400 MHz, CDCl₃) 1.89 (3H, s), 2.12 (3H, s), 4.72 (2H, s), 6.16 (1H, d, J=3 Hz), 6.32 (1H, d, J=3 Hz), 6.54 (1H, d, J=9 Hz), 7.00-7.05 (3H, m), 7.18-7.22 (2H, m), 7.27-7.32 (2H, m), 7.35-7.42 (2H, m), 7.69 (1H, broad s), 7.92 (1H, d, J=8 Hz).

LC/MS t=3.78 min [MH⁺] 495/497.

### Example 97

### 3-{2-[5-Chloro-2-(benzyloxy)-phenyl]-5-methyl-pyrrol-1-yl}-N-[1-3,5-dimethyl-isoxazol-4-yl)-methanoyl-benzenesulfonamide

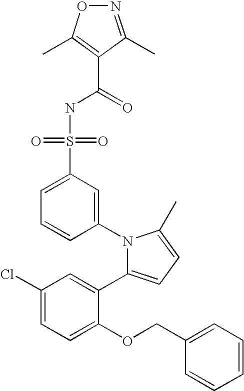

Prepared in the same way as 3-{2-[5-Chloro-2-(benzyloxy)-phenyl]-5-methyl-pyrrol-1-yl}-N-[1-phenyl-methanoyl]-benzenesulfonamide.

LC/MS t=4.51 min, [MH⁺] 576, 578; [MH⁻] 574, 576.

### Example 98

### 3-{2-[5-Chloro-2-(4-fluoro-benzyloxy)-phenyl]-5-methyl-pyrrol-1-yl}-N-acetyl-benzenesulfonamide

### a) 3-{2-[5-Chloro-2-(4-fluoro-benzyloxy)-phenyl]-5-methyl-pyrrol-1-yl}-benzenesulfonamide

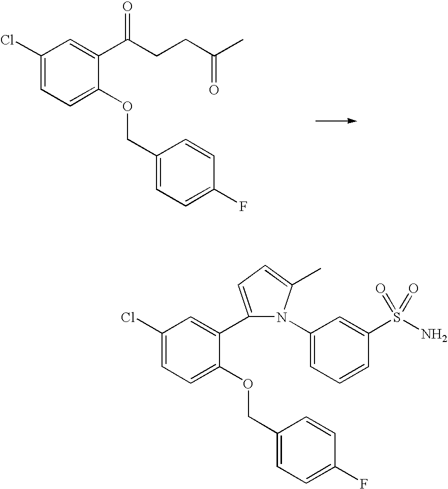

1-[5-Chloro-2-(4-fluoro-benzyloxy)-phenyl]-pentane-1,4-dione (750 mg, 2.25 mmol), 3-amino-benzenesulfonamide (464 mg, 2.69 mmol) and para-toluenesulfonic acid (85 mg, 0.45 mmol) were heated at reflux in toluene (22 ml) for 16 hours. Upon cooling, the mixture was concentrated in vacuo. The residue w was purified by chromatography on silica gel with isohexane/EtOAc (25%) as eluant, to give the title compound (631 mg, 60%).

¹H-NMR (400 MHz, CDCl₃) 2.18 (3H, s), 4.53 (2H, s), 4.71 (2H, s), 6.14 (1H, dd, J=1 Hz, 3 Hz), 6.32 (1H, d, J=3 Hz), 6.57 (1H, d, J=9 Hz), 6.97-7.09 (5H, m), 7.13-7.17 (1H, m), 7.21 (1H, d, J=2.5 Hz), 7.37 (1H, t, J=8 Hz), 7.51 (1H, t, J=0.5 Hz), 7.74-7.79 (1H, m).

LC/MS t=3.75 min [MH⁺]=471/473.

### b) 3-{2-[5-Chloro-2-(4-fluoro-benzyloxy)-phenyl]-5-methyl-pyrrol-1-yl}-N-acetyl-benzenesulfonamide

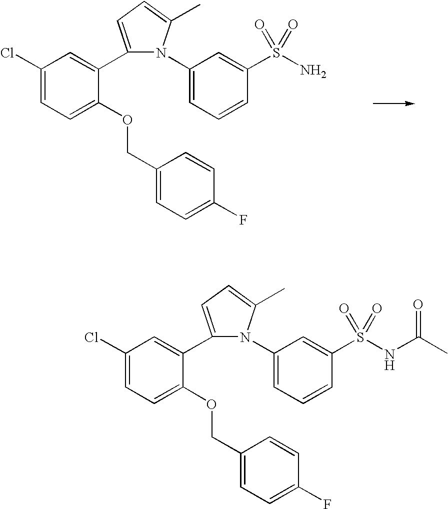

3-{2-[5-Chloro-2-(4-fluoro-benzyloxy)-phenyl]-5-methyl-pyrrol-1-yl}-benzenesulfonamide (0.20g, 0.426 mmol), acetic anhydride (0.044 ml, 0.469 mmol), pyridine (0.069 ml, 0.852 mmol), dimethylaminopyridine (cat.) and DCM (2.5 ml) were stirred at reflux for 30 minutes. The reaction was washed with 2M HCl and the solvent removed in vacuo to yield the title compound as a brown oil (0.249g).

¹H-NMR (400 MHz, CDCl₃) 1.92 (3H, s), 2.18 (3H, s), 4.68 (2H, s), 6.16 (1H, d, J=3 Hz), 6.30 (1H, d, J=3 Hz), 6.53 (1H, d, J=9 Hz), 6.96-7.02 (4H, m), 7.05 (1H, dd, J=3 Hz, 9 Hz), 7.16-7.19 (1H, m), 7.22 (1H, d, J=3 Hz), 7.37 (1H, t, J=8 Hz), 7.67 (1H, broad s), 7.92 (1H, d, J=8 Hz).

LC/MS t=3.80 min [MH⁺] 513/515.

### Example 99

### 3-{2-[5-Chloro-2-(4-fluoro-benzyloxy)-phenyl]-5-methyl-pyrrol-1-yl}-N-(1-phenyl-methanoyl)-benzenesulfonamide

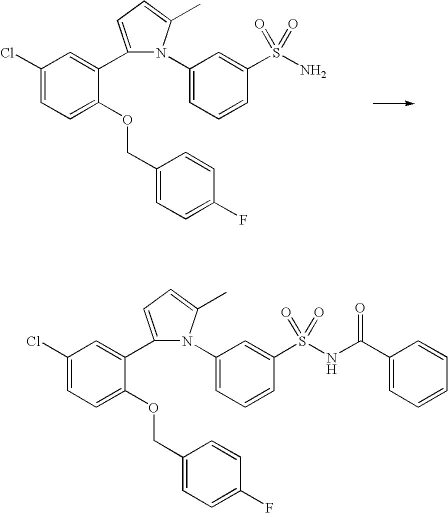

Benzoyl chloride (0.075 ml, 0.63 mmol) was added to 3-{2-[5-chloro-2-(4-fluoro-benzyloxy)-phenyl]-5-methyl-pyrrol-1-yl}-benzenesulfonamide (100 mg, 0.21 mmol), DMAP (26 mg, 0.21 mmol) and triethylamine (0.060 ml, 0.42 mmol) in dichloromethane (1 ml) and stirred at room temperature for 16 hours. The reaction mixture was diluted with dichloromethane, washed with 2M citric acid, brine, dried (MgSO₄), filtered and concentrated. The residue was purified by chromatography on silica gel with isohexane/EtOAc (25%) as eluant, to give the title compound (57 mg, 47%).

¹H-NMR (400 MHz, CDCl₃) 2.18 (3H, s), 4.56 (2H, s), 6.13 (1H, d, J=3.5 Hz), 6.28 (2H, d, J=3.5 Hz), 6.58 (1H, dd, J=2.5, 9 Hz), 6.93-6.98 (4H, m), 7.19 (1H, broad d, J=8 Hz), 7.22 (1H, d, J=3 Hz), 7.39 (1H, t, J=8 Hz), 7.48 (2H, broad t, J=8 Hz), 7.58-7.72 (4H, m), 8.06 (1H, broad d, J=8 Hz), 8.57 (1H, broad s).

LC/MS t=4.28 min [MH⁺] 575/577.

### Example 100

### 3-{2-[5-chloro-2-(2,4-difluoro-benzyloxy)-phenyl]-5-methyl-pyrrol-1-yl}-N-acetyl-benzenesulfonamide

### a) 3-{2-[5-Chloro-2-(2,4-difluoro-benzyloxy)-phenyl]-5-methyl-pyrrol-1-yl}-benzenesulfonamide

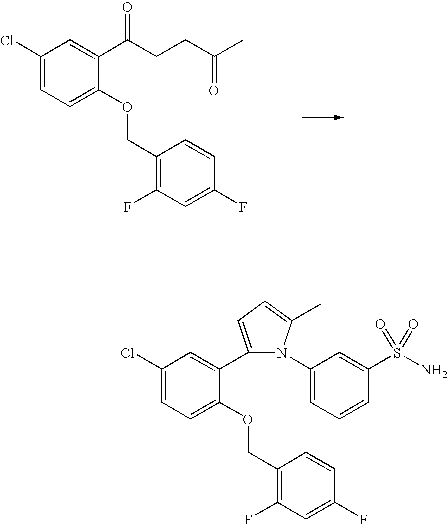

1-[5-Chloro-2-(2,4-difluoro-benzyloxy)-phenyl]-pentane-1,4-dione (1.5g, 4.0 mmol), 3-amino-benzenesulfonamide (881 mg, 5.0 mmol) and para-toluenesulfonic acid (162 mg, 0.85 mmol) were heated at 110° C. in toluene (43 ml) for 16 hours. Upon cooling, the mixture was concentrated in vacuo. The residue was purified by chromatography on silica gel with isohexane/EtOAc (30%) as eluant, to give the title compound (1.53g, 74%).

¹H-NMR (400 MHz, CDCl₃) 2.18 (3H, s), 4.60 (2H, s), 4.76 (2H, s), 6.14 (1H, d, J=3 Hz), 6.31 (1H, d, J=3 Hz), 6.62 (1H, d, J=9 Hz), 6.77-6.87 (2H, m), 6.96-7.05 (1H, m), 7.09 (1H, dd, J=2.5 Hz, 9 Hz), 7.15-7.20 (2H, m), 7.39 (1H, t, J=8 Hz), 7.57 (1H, t, J=0.5 Hz), 7.78 (1H, dt, J=0.5 Hz, 8 Hz).

LC/MS t=3.76 min [MH⁺]=489/491.

### b) 3-{2-[5-Chloro-2-(2,4-difluoro-benzyloxy)-phenyl]-5-methyl-pyrrol-1-yl}-N-acetyl-benzenesulfonamide

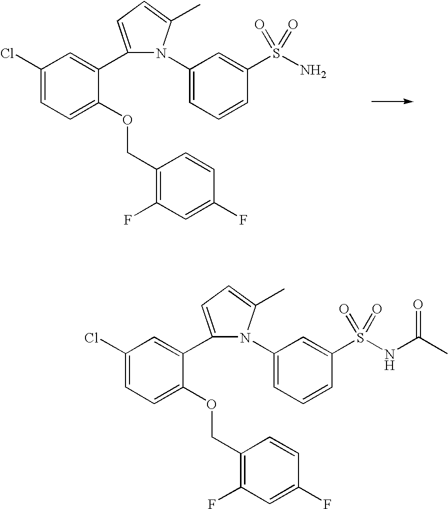

3-{2-[5-Chloro-2-(2,4-difluoro-benzyloxy)-phenyl]-5-methyl-pyrrol-1-yl}-benzenesulfonamide (0.20g, 0.41 0 mmol), acetic anhydride (0.043 ml, 0.485 mmol), pyridine (0.066 ml, 0.820 mmol), dimethylaminopyridine (cat.) and DCM (2.5 ml) were stirred at reflux for 30 minutes. The reaction was washed with 2M HCl and the solvent removed in vacuo to yield the title compound as a brown oil (0.249g).

¹H-NMR (400 MHz, CDCl₃) 1.97 (3H, s), 2.18 (3H, s), 4.75 (2H, s), 6.14 (1H, d, J=3 Hz), 6.30 (1H, d, J=3 Hz), 6.60 (1H, d, J=9 Hz), 6.77-6.84 (2H, m), 6.92-7.00 (1H, m), 7.08 (1H, dd, J=3 Hz, 9 Hz), 7.18 (1H, d, J=3 Hz), 7.22-7.25 (1H, m), 7.42 (1H, t, J=8 Hz), 7.71 (1H, broad s), 7.92 (1H, d, J=8 Hz).

LC/MS t=3.82 min [MH⁺] 531/533.

### Example 101

### 3-{2-[5-Chloro-2-(2,4-difluoro-benzyloxy)-phenyl]-5-methyl-pyrrol-1-yl}-N-(1-phenyl-methanoyl)-benzenesulfonamide

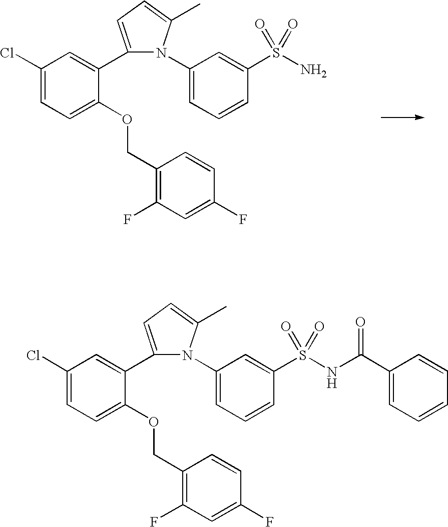

Benzoyl chloride (0.030 ml, 0.25 mmol) was added to 3-{2-[5-chloro-2-(2,4-difluoro-benzyloxy)-phenyl]-5-methyl-pyrrol-1-yl}-benzenesulfonamide (104 mg, 0.21 mmol), DMAP (26 mg, 0.21 mmol) and triethylamine (0.035 ml, 0.25 mmol) in dichloromethane (1 ml) and stirred at room temperature for 16 hours. The reaction mixture was diluted with dichloromethane, washed with 2M citric acid, brine, dried (MgSO₄), filtered and concentrated. The residue was purified by chromatography on silica gel with isohexane/EtOAc (15-25%) as eluant, to give the title compound (102 mg, 81%).

¹H-NMR (400 MHz, CDCl₃) 2.20 (3H, s), 4.67 (2H, s), 6.13 (1H, d, J=3.5 Hz), 6.27 (1H, d, J=3.5 Hz), 6.34 (1H, d, J=9 Hz), 6.66 (1H, dd, J=3, 9 Hz), 6.69-6.90 (3H, m), 7.19 (1H, d, J=2.5 Hz), 7.24 (1H, broad s), 7.40-7.51 (3H, m), 7.59-7.65 (1H, m), 7.69 (2H, broad d, J=8 Hz), 7.78 (1H, broad s), 8.06 (1H, broad d, J=8 Hz), 8.60 (1H, broad s).

LC/MS t=4.31 min [MH⁺] 593/595.

### Example 102

### 4-{2-[5-Chloro-2-(4-fluoro-benzyloxy)-phenyl]-5-methyl-pyrrol-1-yl}-N-(1-phenyl-methanoyl)-benzenesulfonamide

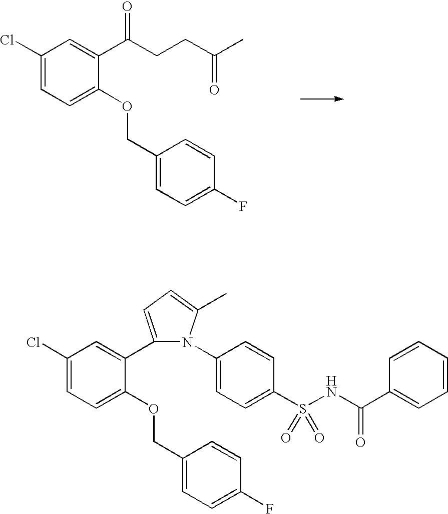

1-[5-Chloro-2-(4-fluoro-benzyloxy)-phenyl]-pentane-1,4-dione (100 mg, 0.30 mmol), 4-amino-N-(1-phenyl-methanoyl)-benzenesulfonamide (0.99 mg, 0.36 mmol) and para-toluenesulfonic acid (1 mg, 0.06 mmol) were heated at reflux in toluene (3 ml) for 2 hours. Upon cooling, the mixture was concentrated. The residue was purified by chromatography on silica gel with isohexane/EtOAc (35%) as eluant, to give the title compound (87 mg, 51%).

¹H-NMR (400 MHz, CDCl₃) 2.15 (3H, s), 4.50 (2H, s), 6.13 (1H, broad d, J=3 Hz), 6.29 (1H, dd, J=1, 3 Hz), 6.50 (1H, d, J=9 Hz), 6.96-7.09 (8H, m), 7.47 (2H, broad t, J=8 Hz), 7.61 (1H, broad t, J=8 Hz), 7.78 (2H, broad d, J=8 Hz), 7.95 (2H, broad d, J=8 Hz).

LC/MS t=4.29 min [MH⁺] 575/577.

### Example 103

### 4-{2-[5-Chloro-2-(2,4-difluoro-benzyloxy)-phenyl]-5-methyl-pyrrol-1-yl}-N-(1-phenyl-methanoyl)-benzenesulfonamide

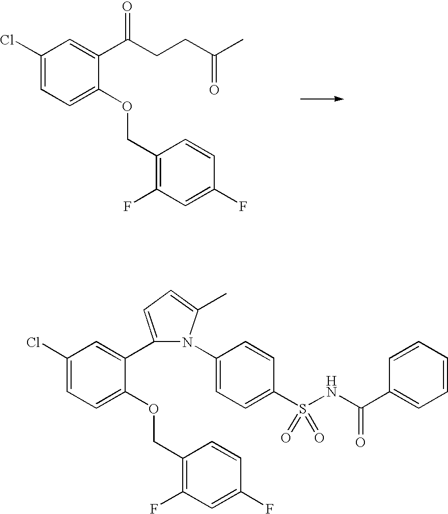

1-[5-Chloro-2-(2,4-difluoro-benzyloxy)-phenyl]-pentane-1,4-dione (103 mg, 0.30 mmol), 4-amino-N-(1-phenyl-methanoyl)-benzenesulfonamide (99 mg, 0.36 mmol) and para-toluenesulfonic acid (11 mg, 0.06 mmol) were heated at reflux in toluene (3 ml) for 2 hours. Upon cooling, the mixture was concentrated. The residue was purified by chromatography on silica gel with isohexane/EtOAc (35%) as eluant, to give the title compound (126 mg, 71%).

¹H-NMR (400 MHz, CDCl₃) 2.16 (3H, s), 4.62 (2H, s), 6.12 (1H, d, J=3.5 Hz), 6.29 (1H, d, J=3.5 Hz), 6.56 (1H, d, J=9 Hz), 6.75-6.88 (2H, m), 6.95-7.10 (4H, m), 7.21 (1H, d, J=3 Hz), 7.45 (2H, broad t, J=8 Hz), 7.59 (1H, broad t, J=8 Hz), 7.77 (2H, broad d, J=8 Hz), 8.00 (2H, d, J=8 Hz), 8.88 (1H, broad s).

LC/MS t=4.26 min [MH⁺] 593/595.

### Example 104

### 3-{2-[5-Chloro-2-(benzyloxy)-phenyl]-5-methyl-pyrrol-1yl}-N-[(R)-1-phenyl-ethyl]-benzamide

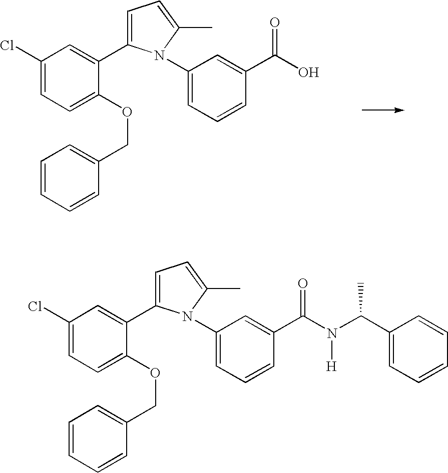

3-{2-[5-Chloro-2-(benzyloxy)-phenyl]-5-methyl-pyrrol-1yl}-benzoic acid (0.12g, 0.575 mmol, 1 eq) was dissolved in DCM (2.5 ml), EDAC (0.0715g, 0.747 mmol, 1.3 eq) and HOBt (0.0505g, 0.747 mmol, 1.3 eq) were added to the reaction vessel and stirred for 5 min at 21° C. (R)-Phenylethylamine (0.139g, 0.1 15 mmol, 2 eq) was then added and stirred for 6 hours at room temperature The reaction mixture was then diluted with ethyl acetate and washed with saturated NH₄Cl and saturated NaHCO₃. The combined organic extracts were washed with brine and dried (MgSO₄), filtered and volatiles removed in vacuo to yield title compound (0.107g, 0.206 mmol, 36%) as a clear yellow oil.

LC/MS t=4.10 min [MH+] 521/523.

### Example 105

### 3-{2-[5-Chloro-2-(benzyloxy)-phenyl]-5-methyl-pyrrol-1yl}-N-methyl-N-[(R)-1-phenyl-ethyl]-benzamide

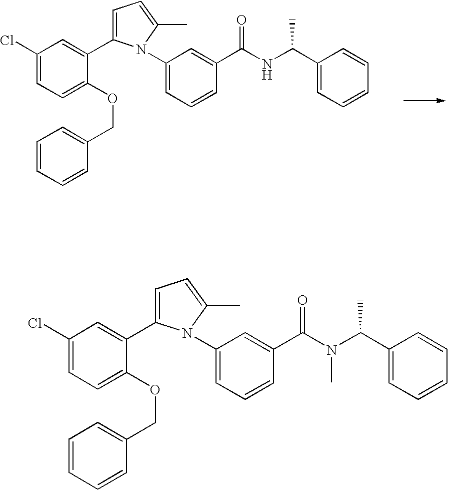

3-{2-[5-Chloro-2-(benzyloxy)-phenyl]-5-methyl-pyrrol-1yl}-N-[(R)-1-phenyl-ethyl]-benzamide (0.050g, 0.096 mmol, 1 eq), was dissolved in DMF (1 ml) and sodium hydride (6 mg, 0.24 mmol, 1.5 eq) was added at 0° C. and stirred for 1 hour. Methyliodide (0.0065 ml, 0.021 mmol, 1.1 eq) was then added and the reaction allowed to warm to room temperature with stirring for 2 hours. The volatiles were removed in vacuo the reaction mixture was diluted with EtOAc, and washed with water. The combined organic extracts were washed with brine and dried (MgSO₄), filtered and volatiles removed in vacuo to yield the title compound (0.021g, 82%) as a yellow solid.

LC/MS t=4.13 min [MH+] 535/537.

### Example 106

### 3-{2-[5-Chloro-2-(benzyloxy)-phenyl]-5-methyl-pyrrol-1yl}-N-[(S)-1-phenyl-ethyl]-benzamide

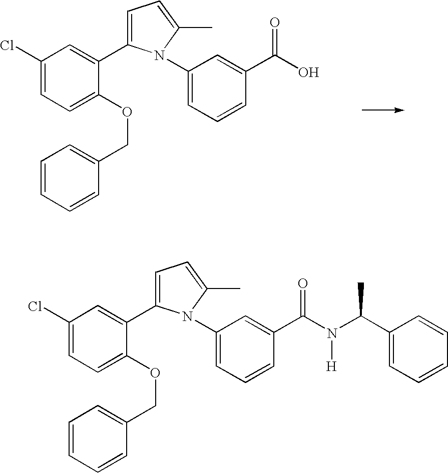

Procedure as for 3-{2-[5-chloro-2-(benzyloxy)-phenyl]-5-methyl-pyrrol-1yl}-N-[(R)-1-phenyl-ethyl]-benzamide using the appropriate benzoic acid.

LC/MS t=4.08 min [MH+] 521/523.

### Example 107

### 3-{2-[5-Chloro-2-(benzyloxy)-phenyl]-5-methyl-pyrrol-1yl}-N-methyl-N-[(S)-1-phenyl-ethyl-benzamide

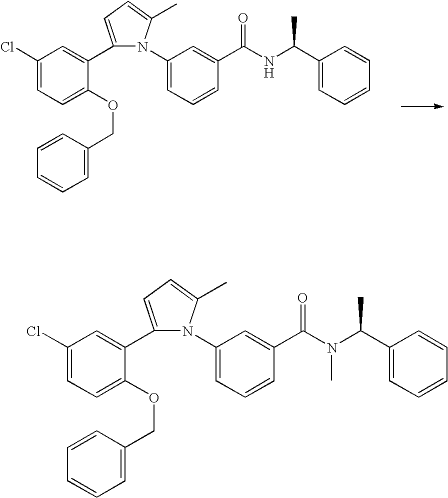

Procedure as for 3-{2-[5-Chloro-2-(benzyloxy)-phenyl]-5-methyl-pyrrol-1yl}-N-methyl-N-[(R)-1-phenyl-ethyl]-benzamide using the appropriate benzamide.

LC/MS t=4.13 min [MH+] 535/537.

### Example 108

### 4-{2-[5-Chloro-2-(benzyloxy)-phenyl]-5-methyl-pyrrol-1yl}-N-[(R)-1-phenyl-ethyl]-benzamide

### a) 4-{2-[5-Chloro-2-(benzyloxy)-phenyl]-5-methyl-pyrrol-1yl)--benzoic acid ethyl ester

1-[5-Chloro-2-(benzyloxy)-phenyl]-pentane-1,4-dione (0.50g, 1.58 mmol, 1 eq) was heated with ethyl-3-aminobenzoate (0.313g, 1.90 mmol, 1.2 eq), in a sealed vessel, to 150° C. for 24 hours. The reaction mixture was allowed to warm to room temp and diluted with ethylacetate and washed with 2M HCl, the combined organics were washed with brine and dried (MgSO₄) and volatiles removed in-vacuo. The residue was purified by chromatography on silica gel with 5% EtOAc:iso-hexane as the eluant to yield the title compound (0.32g, 0.72 mmol, 45%) as a pale yellow solid.

LC/MS t=4.23 min [MH+] 446/448.

### b) 4-{2-[5-Chloro-2-(benzoxy)-phenyl]-5-methyl-pyrrol-1yl}-benzoic acid

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

LC/MS t=3.98 min [MH+] 432/434

### c) 4-[2-(5-Chloro-2-benzyloxy-phenyl)-5-methyl-pyrrol-1yl]-N-[(R)-1-phenyl-ethyl]-benzamide

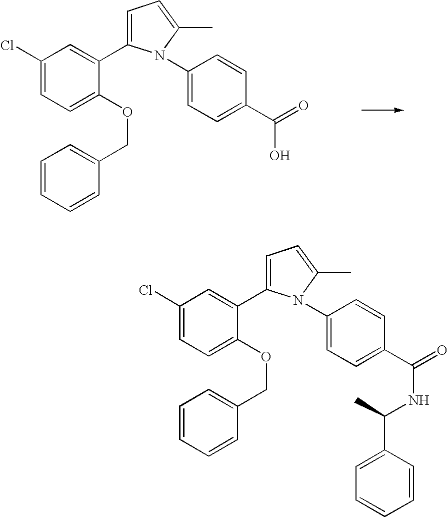

Procedure as for 3-{2-[5-chloro-2-(benzyloxy)-phenyl]-5-methyl-pyrrol-1yl}-N-[(R)-1-phenyl-ethyl]-benzamide using the appropriate benzoic acid.

¹H NMR (400 MHz, CDCl₃) 1.61 (3H, d, J=7 Hz), 2.13 (3H, s), 4.71 (2H, s), 5.32 (1H, m), 6.12 (1H, d, J=3 Hz), 6.23 (1H, d, J=8 Hz), 6.30 (1H, d, J=3 Hz), 6.54 (1H, d, J=8H), 6.95-7.07 (5H, m), 7.20-7.41 (9H, m), 7.60 (1H, d, J=8 Hz).

LC/MS t=4.09 min [MH+] 521/523.

### Example 109

### 4-{2-[5-Chloro-2-(benzyloxy)-phenyl]-5-methyl-pyrrol-1yl}-N-[(S)-1-phenyl-ethyl]-benzamide

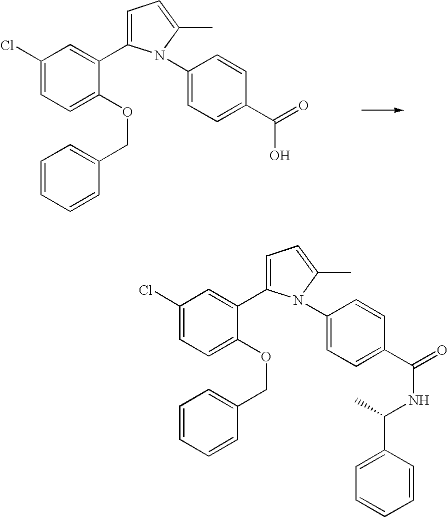

Procedure as for 3-{2-[5-chloro-2-(benzyloxy)-phenyl]-5-methyl-pyrrol-1yl}-N-[(R)-1-phenyl-ethyl]-benzamide using the appropriate benzoic acid.

¹H NMR (400 MHz, CDCl₃) 1.61 (3H, d, J=7 Hz), 2.13 (3H, s), 4.71 (2H, s), 5.23-5.42 (1H, m), 6.12 (1H, d, J=3 Hz), 6.23 (1H, d, J=8 Hz), 6.30 (1H, d, J=3 Hz), 6.54 (1H, d, J=8H), 6.95-7.07 (5H, m), 7.20-7.41 (9H, m), 7.60 (1H, d, J=8 Hz).

LC/MS t=4.09 min [MH+] 521/523.

### Example 110

### 3-{2-[5-Bromo-2-(4-fluoro-benzyloxy)-phenyl]-5-methyl-pyrrol-1yl}-5-acetylamino-N-[(S)-1-phenyl-ethyl]-benzamide

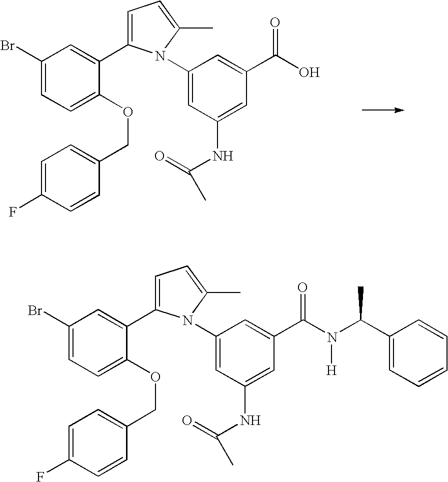

Procedure as for 3-{2-[5-chloro-2-(benzyloxy)-phenyl]-5-methyl-pyrrol-1yl}-N-[(R)-1-phenyl-ethyl]-benzamide using the appropriate benzoic acid.

LC/MS t=3.90 min [MH+] 640/642.

### Example 111

### 3-}2-[5-Bromo-2-(4-fluoro-benzyloxy)-phenyl]-5-methyl-pyrrol-1yl}-6-chloro-N-[(S)-1-phenyl-ethyl]-benzamide

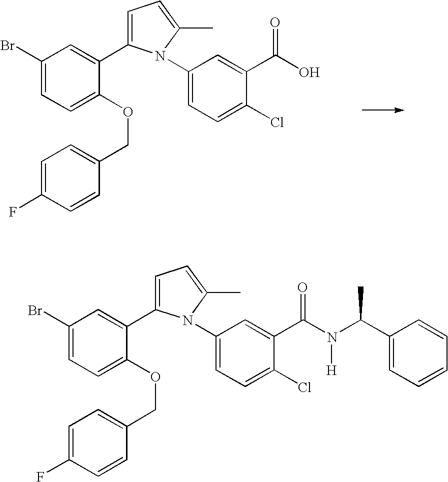

Procedure as for 3-{2-[5-chloro-2-(benzyloxy)-phenyl]-5-methyl-pyrrol-1yl}-N-[(R)-1-phenyl-ethyl]-benzamide using the appropriate benzoic acid.

¹H NMR (400 MHz, CDCl₃) 1.51 (3H, d, J=7 Hz), 2.143 (3H, s), 4.72 (2H, s), 5.17-5.26 (1H, m), 5.89 (1H, d, J=8 Hz), 6.11 (1H, d, J=3 Hz), 6.27 (1H, d, J=3 Hz), 6.47 (1H, d, J=8H), 6.88-6.99 (5H, m), 7.16 (1H, dd, J=8 Hz, 2 Hz), 7.22-7.40 (7H, m).

LC/MS t=4.16 min [MH+] 617/619.

### Example 112

### 3-{2-[5-Bromo-2-(2,4-difluoro-benzyloxy)-phenyl]-5-methyl-pyrrol-1yl}-5-acetylamino-N-[(S)-1-phenyl-ethyl]-benzamide

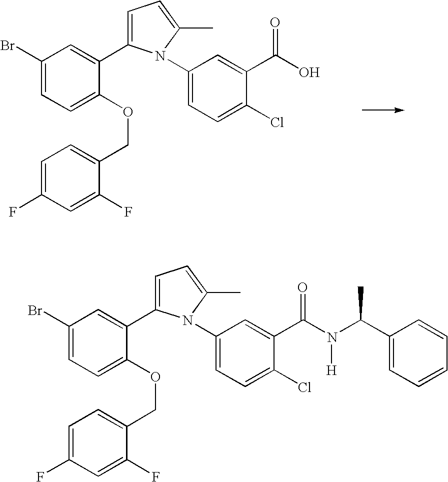

Procedure as for 3-{2-[5-chloro-2-(benzyloxy)-phenyl]-5-methyl-pyrrol-1 yl)N-[(R)-1-phenyl-ethyl]-benzamide using the appropriate benzoic acid.

¹H NMR (400 MHz, CDCl₃) 1.53 (3H, d, J=7 Hz), 2.15 (3H, s), 4.78 (2H, s), 5.19-5.27 (1H, m), 6.02 (1H, d, J=8 Hz), 6.10 (1H, d, J=3 Hz), 6.27 (1H, d, J=3 Hz), 6.52 (1H, d, J=8H), 6.70-6.98 (5H, m), 7.18 (1H, dd, J=8 Hz, 2 Hz), 7.22-7.40 (6H, m).

LC/MS t=4.18 min [MH+] 635/637.

### Example 113

### 4-{2-[5-Bromo-2-(2,4-difluoro-benzyloxy)-phenyl]-5-methyl-pyrrol-1-yl]-benzamide

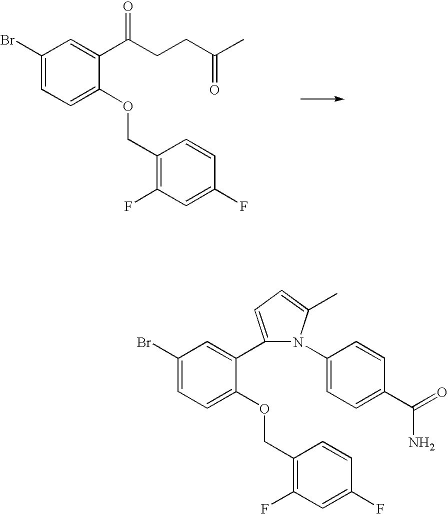

1-[5-Bromo-2-(2,4-difluoro-benzyloxy)-phenyl]-pentane-1,4-dione (0.10g, 0.25 mmol), 4-amino-benzamide (0.041g, 0.3 mmol) and p-TSA (0.009g, 0.05 mmol) were refluxed in toluene (1.5 ml) for 18 hours under a nitrogen atmosphere. The solvent was removed in vacuo and the resultant residue purified by MDAP. This yielded a white solid (0.013g, 10.4%)

¹H-NMR (400 MHz, CDCl₃,) 2.16 (3H, s), 4.72 (2H, s), 6.13 (1H, d, J=3 Hz), 6.29 (1H, d, J=3 Hz), 6.55 (1H, d, J=9 Hz), 6.77-6.84 (2H, m), 6.89-6.96 (1H, m), 7.03 (2H, d, J=9 Hz), 7.23 (1H, dd, J=3 Hz, 9 Hz), 7.36 (1H, d, J=3 Hz), 7.68 (2H, d, J=9 Hz).

LC/MS t=3.75 min [MH⁺] 497/499.

### Example 114

### 4-{2-[6-Bromo-2-(2,4-difluoro-benzyloxy)-phenyl]-5-methyl-pyrrol-1-yl}-N-methyl-benzamide

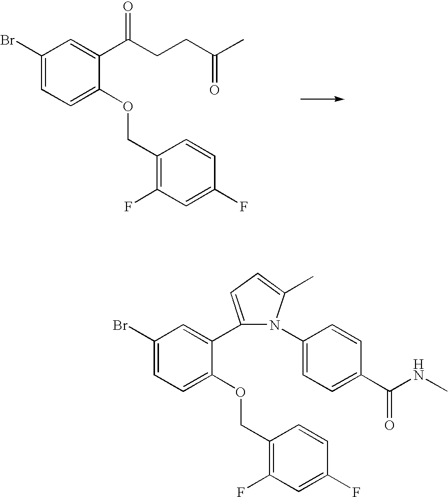

Procedure as for 4-{2-[5-bromo-2-(2,4-difluoro-benzyloxy)-phenyl]-5-methyl-pyrrol-1-yl}-benzamide using the appropriate amine to give title compound.

¹H-NMR (400 MHz, CDCl₃) 2.13 (3H, s), 3.01 (3H, d, J=5 Hz), 4.72 (2H, s), 6.03-6.09 (1H, m), 6.12 (1H, d, J=3 Hz), 6.29 (1H, d, J=3 Hz), 6.55 (1H, d, J=9 Hz), 6.80 (2H, t, J=9 Hz), 6.92-6.99 (1H, m), 7.01 (2H, d, J=9 Hz), 7.22 (1H, dd, J=3 Hz, 9 Hz), 7.34 (1H, d, J=3 Hz), 7.62 (2H, d, J=9 Hz).

LC/MS t=3.86 min [MH⁺] 511/513.

### Example 115

### 4-{2-[5-Bromo-2-(2,4-difluoro-benzyloxy)-phenyl]-5-methyl-pyrrol-1-yl}-N,N-dimethyl-benzamide

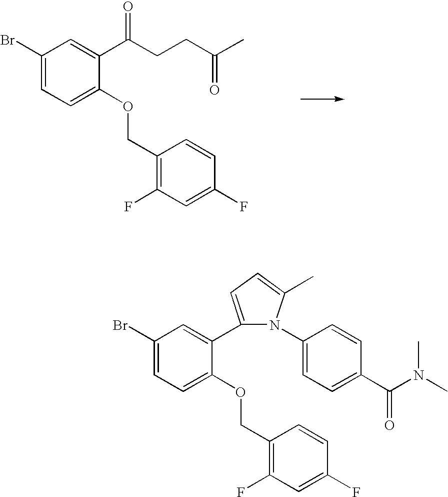

Procedure as for 4-{2-[5-bromo-2-(2,4-difluoro-benzyloxy)-phenyl]-5-methyl-pyrrol-1-yl}-benzamide using the appropriate amine to give title compound.

LC/MS t=3.91 min [MH⁺] 525/527.

### Example 116

### 4-{2-[5-Bromo-2-(2,4-difluoro-benzyloxy)-phenyl]-5-methyl-pyrrol-1-yl}-N,N-diethyl-benzamide

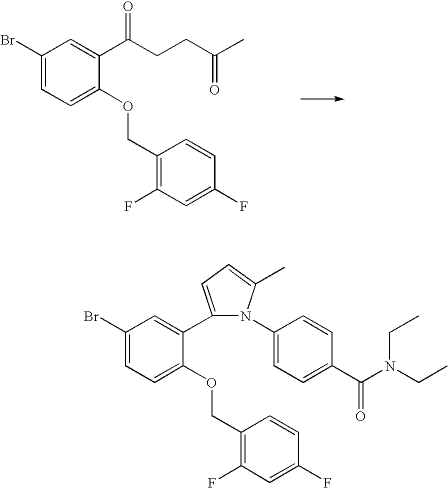

Procedure as for 4-{2-[5bromo-2-(2,4-difluoro-benzyloxy)-phenyl]-5-methyl-pyrrol-1-yl}benzamide using the appropriate amine to give title compound.

LC/MS t=4.09 min [MH⁺] 553/555.

### Example 117

### 4-{2-[5-Bromo-2-(2,4-difluoro-benzyloxy)-phenyl]-5-methyl-pyrrol-1-yl}-N-tert-butyl-benzamide

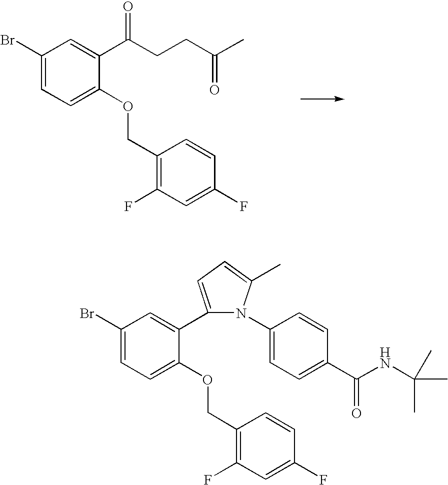

Procedure as for 4-{2-[5-bromo-2-(2,4-difluoro-benzyloxy)-phenyl]-5-methyl-pyrrol-1-yl}-benzamide using the appropriate amine to give title compound.

LC/MS t=4.18 min [MH⁺] 553/555.

### Example 118

### 3-{2-[5-Bromo-2-(2,4-difluoro-benzyloxy)-phenyl]-5-methyl-pyrrol-1-yl}-N-methyl-benzamide

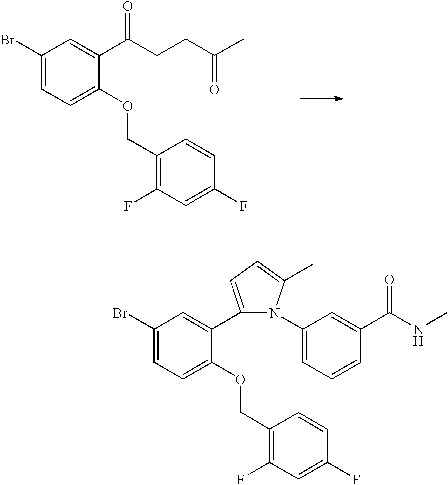

Procedure as for 4-{2-[5-bromo-2-(2,4-difluoro-benzyloxy)-phenyl]-5-methyl-pyrrol-1-yl}-benzamide using the appropriate amine to give title compound.

LC/MS t=3.86 min [MH⁺] 511/5136.

### Example 119

### 3-{2-[5-Bromo-2-(2,4-difluoro-benzyloxy)-phenyl]-5-methyl-pyrrol-1-yl}-N,N-dimethyl-benzamide

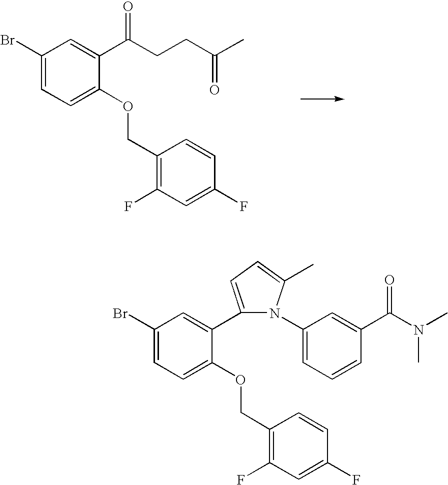

Procedure as for 4-{2-[5-bromo-2-(2,4-difluoro-benzyloxy)-phenyl]-5-methyl-pyrrol-1-yl}-benzamide using the appropriate amine to give title compound.

¹H-NMR (400 MHz, CDCl₃) 2.15 (3H, s), 2.56 (3H, s), 3.02 (3H, s), 4.82 (2H, s), 6.10 (1H, d, J=3 Hz), 6.33 (1H, d, J=3 Hz), 6.59 (1H, d, J=9 Hz), 6.78-6.86 (2H, m), 6.91-6.94 (1H, m), 7.06-7.13 (2H, m), 7.17 (1H, dd, J=3 Hz, 9 Hz), 7.24 (1H, d, J=3 Hz), 7.31-7.38 (2H, m).

LC/MS t=3.90 min [MH⁺] 525.

### Example 120

### 3-{2-[5-Bromo-2-(2,4-difluoro-benzyloxy)-phenyl]-5-methyl-pyrrol-1-yl}-N-(1-methylsulfonyl)-benzamide

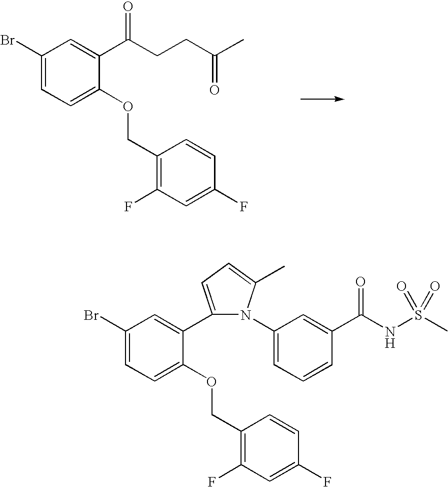

Preparation as for 2-(3-{2-[5-bromo-2-(2,4-difluorobenzyloxy)-phenyl]-5-methyl-pyrrol-1-yl}-phenyl)-1H-benzoimidazole (81 mg, 56%) using the appropriate amine (JP6042371).

¹H NMR (400 MHz, CDCl₃) 2.14 (3H, s), 3.40 (3H, s), 4.76 (2H, s), 6.13 (1H, d, J=3 Hz), 6.30 (1H, d, J=3 Hz), 6.62 (1H, d, J=8 Hz), 6.82 (2H, m), 7.06 (1H, m), 7.23 (2H, m), 7.31 (1H, d, J=3 Hz), 7.38 (1H, t, J=8 Hz), 7.49 (1H, bs), 7.67 (1H, bd, J=8 Hz), 8.21 (1H, s).

LC/MS t=4.04 min [MH+] 575/577.

### Example 121

### 3-{2-[5-Bromo-2-(2,4-difluoro-benzyloxy)-phenyl]-5-methyl-pyrrol-1-yl}-N-(1H-tetrazol-5-yl)-benzamide

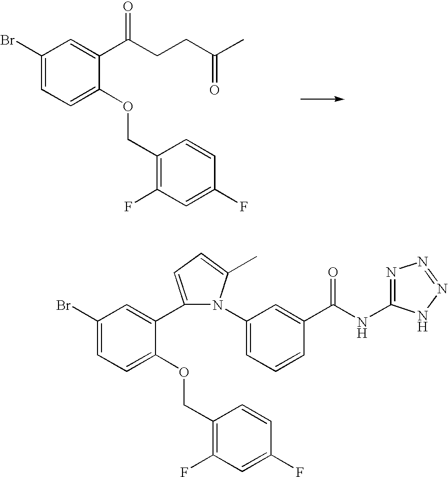

Preparation as for 2-(3-{2-[5-bromo-2-(2,4-difluoro-benzyloxy)-phenyl]-5-methyl-pyrrol-1-yl}-phenyl)-1H-benzoimidazole using the appropriate amine (Oku et al, WO9613485) (17 mg, 12%).

¹H NMR (400 MHz, CDCl₃) 2.17 (3H,s), 4.75(2H, s), 6.14 (1H, d, J=3 Hz), 6.31 (1H, d, J=3 Hz), 6.58 (1H, d, J=8 Hz), 6.70-6.84(2H, m), 7.01-7.09 (1H, m), 7.20-7.25 (2H, m), 7.30-7.35 (2H, m), 7.43 (1H, t, J=8 Hz), 7.75 (1H, s), 8.00 (1H, d, J=8 Hz)

### Example 122

### 4-{2-[5-Bromo-2-(2,4-difluoro-benzyloxy-phenyl]-5-methyl-pyrrol-1-yl}-N-pyridin-2-yl-benzamide

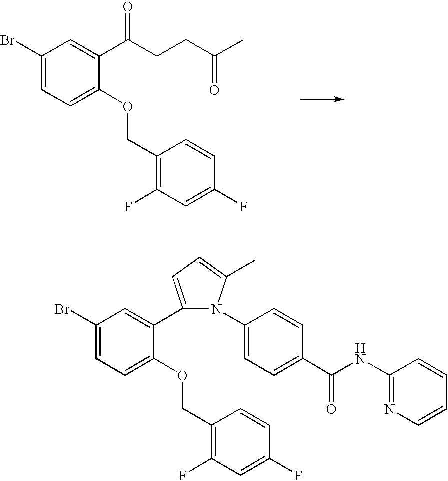

Preparation as for 2-(3-{2-[5-bromo-2-(2,4difluoro-benzyloxy)-phenyl]-5-methyl-pyrrol-1-yl}-phenyl)-1H-benzoimidazole using the appropriate amine (23 mg, 16%).

¹H NMR (400 MHz, CDCl₃) 2.18 (3H, s), 4.71 (2H, s), 6.13 (1H, m), 6.30 (1H, d, J=4 Hz), 6.57 (1H, d, J=10 Hz), 6.77-6.85 (2H, m), 6.94-7.02 (1H, m), 7.04-7.10 (2H, m), 7.13-7.18 (1H, m), 7.22-7.28 (1H, m), 7.38 (1H, d, J=2 Hz), 7.82-7.90 (3H, m), 8.25-8.30 (1H, m), 8.49 (1H, d, J=8 Hz), 9.34 (1H, bs).

LC/MS t=4.11 min [MH+] 574/576.

### Example 123

### 2-{2-[5-Chloro-2-(4-methoxy-benzyloxy)-phenyl]-5-methyl-pyrrol-1-yl}-isonicotinic acid

### a) 2-{2-[5-Chloro-2-(4-methoxy-benzyloxy)-phenyl]-5-methyl-pyrrol-1-yl}-isonicotinic acid ethyl ester

1-[5-Chloro-2-(4-methoxy-benzyloxy)-phenyl]-pentane-1,4-dione (1.04g, 2.9 mmol) and 2-amino-isonicotinic acid ethyl ester (0.54g, 3.2 mmol) (Linschoten et al, WO0066557) were heated in toluene (0.5 ml) in a sealed vessel at 150° C. for 12 hours. Upon cooling, the residue was purified by chromatography on silica gel with isohexane/EtOAc (15%) as eluant, to give the title compound (510 mg, 36%).

¹H NMR (400 MHz, CDCl₃) 1.31 (3H, t, J=7.5 Hz), 2.29 (3H, s), 3.79 (3H, s), 4.29 (2H, q, J=7.5 Hz), 4.59 (2H, s), 6.12 (1H, d, J=3.5 Hz), 6.32 (1H, d, J=3.5 Hz), 6.58 (1H, broad d, J=9 Hz), 6.79 (2H, d, J=8.5 Hz), 6.98 (2H, d, J=8.5 Hz), 7.05 (1H, dd, J=3, 9 Hz), 7.26 (1H, d, under CDCl₃), 7.40 (1H, broad s), 7.66 (1H, dd, J=1.5, 7 Hz), 8.52 (1H, d, J=7 Hz).

LC/MS t=4.01 min [MH+] 477/479

### b) 2-{2-[5-Chloro-2-(4-methoxy-benzyloxy)-phenyl]-5-methyl-pyrrol-1-yl}-isonicotinic acid

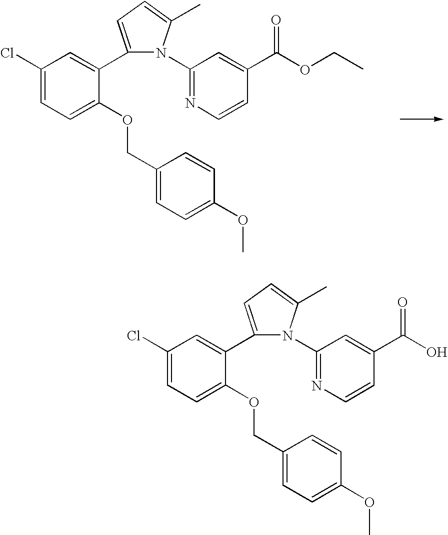

2-{2-[5-Chloro-2-(4-methoxy-benzyloxy)-phenyl]-5-methyl-pyrrol-1-yl}-isonicotinic acid ethyl ester (30 mg, 0.06 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (20 mg, 71%).

¹H NMR (400 MHz, d₆-DMSO) 2.19 (3H, s), 3.73 (3H, s), 4.64 (2H, s), 6.07 (1H, broad d, J=3 Hz), 6.25 (1H, d, J=3 Hz), 6.83-6.88 (3H, m), 7.02 (2H, d, J=9 Hz), 7.08 (1H, d, J=3 Hz), 7.19 (1H, dd, J=3, 9 Hz), 7.32 (1H, s), 7.71 (1H, dd, J=1.5, 5 Hz), 8.58 (1H, d, J=5 Hz), 13.62 (1H, broad s).

LC/MS t=4.00 min [MH−] 447/449

### Example 124

### 2-{2-[5-Chloro-2-benzyloxy)-phenyl]-5-methyl-pyrrol-1-yl}-isonicotinic acid

### a) 2-{2-[5-Chloro-2-(hydroxy)-phenyl]-5-methyl-pyrrol-1-yl}-isonicotinic acid ethyl ester

HCl (4M in dioxane, 2.5 ml, 10 mmol) was added to 2-{2-[5-chloro-2-(4-methoxy-benzyloxy)-phenyl]-5-methyl-pyrrol-1-yl)isonicotinic acid (480 mg, 1 mmol) and stirred at room temperature for 15 minutes. The reaction was concentrated and the residue partitioned between CH₂Cl₂ and NaHCO₃. The organics were washed with brine, dried (MgSO₄), filtered and concentrated. The residue was purified by chromatography on silica gel with isohexane/EtOAc (20-30%) as eluant, to give the title compound (300 mg, 84%).

¹H NMR (400 MHz, CDCl₃) 1.37 (3H, t, J=7 Hz), 2.24 (3H, s), 4.38 (2H, q, J=7 Hz), 6.17 (1H, broad s), 6.27 (1H, d, J=3.5 Hz), 6.84 (1H, d, J=9 Hz), 6.93 (1H, broad s), 7.09 (1H, broad d, J=9 Hz), 7.30 (1H, broad d, J=9 Hz), 7.47 (1H, broad s), 7.62 (1H, broad s), 7.82 (1H, broad d, J=5 Hz).

LC/MS t=3.56 min [MH+] 357/359

### b) 2-{2-[5-Chloro-2-(benzyloxy)-phenyl]-5-methyl-pyrrol-1-yl}-isonicotinic acid ethyl ester

Benzyl bromide (0.013 ml, 0.11 mmol) was added to 2-{2-[5-chloro-2-(hydroxy)-phenyl]-5-methyl-pyrrol-1-yl}-isonicotinic acid ethyl ester (40 mg, 0.11 mmol) and K₂CO₃ (31 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (35 mg, 70%).

¹H NMR (400 MHz, CDCl₃) 1.30 (3H, t, J=7 Hz), 2.30 (3H, s), 4.29 (2H, q, J=7 Hz), 4.67 (2H, s), 6.13 (1H, d, J=3 Hz), 6.33 (1H, d, J=3 Hz), 6.56 (1H, d, J=9 Hz), 7.02-7.07 (3H, m), 7.22-7.29 (4H, m), 7.40 (1H, s), 7.66 (1H, dd, J=1.5, 5 Hz), 8.52 (1H, d, J=5 Hz).

LC/MS t=4.03 min [MH+] 447/449

### c) 2-{2-[5-Chloro-2-(benzyloxy)-phenyl]-5-methyl-pyrrol-1-yl}-isonicotinic acid

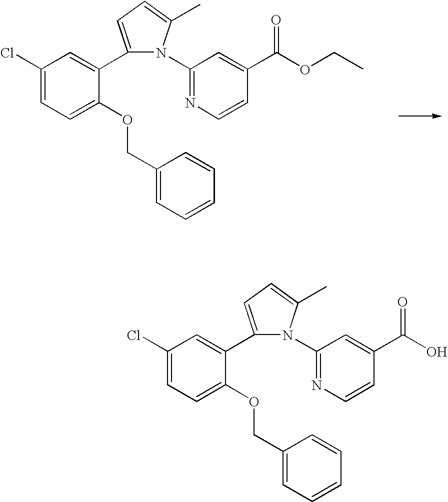

2-{2-[5-Chloro-2-(benzyloxy)-phenyl]-5-methyl-pyrrol-1-yl}-isonicotinic acid ethyl ester (35 mg, 0.08 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (28 mg, 85%).

¹H NMR (400 MHz, d6-DMSO) 2.20 (3H, s), 4.73 (2H, s), 6.09 (1H, broad d, J=3.5 Hz), 6.28 (1H, d, J=3.5 Hz), 6.85 (1H, d, J=9 Hz), 7.06 (2H, dd, J=1.5, 8 Hz), 7.10 (1H, d, J=3 Hz), 7.19 (1H, dd, J=3, 9 Hz), 7.25-7.32 (3H, m), 7.33 (1H, s), 7.72 (1H, dd, J=1.5, 5 Hz), 8.58 (1H, d, J=5 Hz), 13.58 (1H, broad s).

LC/MS t=4.02 min [MH+] 419/421

### Example 125

### 2-{2-[5-Chloro-2-(4-chloro-benzyloxy)-phenyl]-5-methyl-pyrrol-1-yl}-isonicotinic acid

### a) 2-{2-[5-Chloro-2-(4-chloro-benzyloxy)-phenyl]-5-methyl-pyrrol-1-yl}-isonicotinic acid ethyl ester

4-Chloro-benzyl bromide (23 mg, 0.11 1 mmol) was added to 2-{2-[5-chloro-2-(hydroxy)-phenyl]-5-methyl-pyrrol-1-yl}isonicotinic acid ethyl ester (40 mg, 0.11 mmol) and K₂CO₃ (31 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (47 mg, 87%).

¹H NMR (400 MHz, CDCl₃) 1.31 (3H, t, J=7 Hz), 2.30 (3H, s), 4.30 (2H, q, J=7 Hz), 4.61 (2H, s), 6.13 (1H, d, J=3.5 Hz), 6.31 (1H, d, J=3.5 Hz), 6.54 (1H, d, J=9 Hz), 6.99 (2H, d, J=8.5 Hz), 7.06 (1H, dd, J=2.5, 9 Hz), 7.24 (2H, d, J=8.5 Hz), 7.29 (1H, d, J=2.5 Hz), 7.38 (1H, s), 7.66 (1H, dd, J=1.5, 5 Hz), 8.50 (1H, d, J=5 Hz).

LC/MS t=4.16 min [MH+] 481/483/485.

### b) 2-{2-[5-Chloro-2-(4-chloro-benzyloxy)-phenyl]-5-methyl-pyrrol-1-yl}-isonicotinic acid

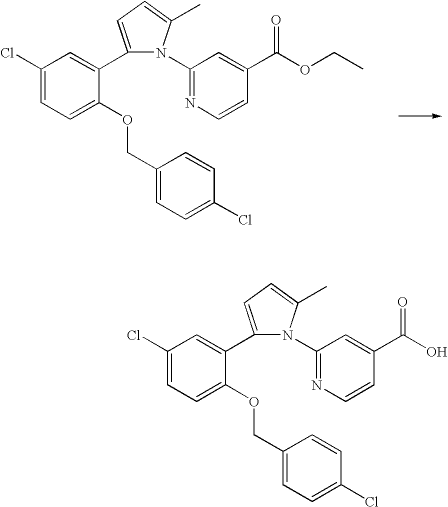

2-{2-[5-Chloro-2-(4-chloro-benzyloxy)-phenyl]-5-methyl-pyrrol-1-yl}-isonicotinic acid ethyl ester (47 mg, 0.10 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (35 mg, 79%).

¹H NMR (400 MHz, d6-DMSO) 2.20 (3H, s), 4.72 (2H, s), 6.09 (1H, dd, J=3.5 Hz), 6.28 (1H, d, J=3.5 Hz), 6.84 (1H, d, J=9 Hz), 7.09 (2H, d, J=8.5 Hz), 7.12 (1H, d, J=3 Hz), 7.20 (1H, dd, J=3, 9 Hz), 7.32 (1H, s), 7.35 (2H, d, J=8.5 Hz), 7.70 (1H, dd, J=1.5, 5 Hz), 8.57 (1H, d, J=5 Hz), 13.50 (1H, broad s).

LC/MS t=4.24 min [MH+] 453/455/457.

### Example 126

### 2-{2-[5-Chloro-2-(4-fluoro-benzyloxy)-phenyl]-5-methyl-pyrrol-1-yl}-isonicotinic acid

### a) 2-{2-[5-Chloro-2-(4-fluoro-benzyloxy)-phenyl]-5-methyl-pyrrol-1-yl}-isonicotinic acid ethyl ester

4-Fluoro-benzyl bromide (0.014 ml, 0.11 mmol) was added to 2-{2-[5-chloro-2-(hydroxy)-phenyl]-5-methyl-pyrrol-1-yl}-isonicotinic acid ethyl ester (40 mg, 0.11 mmol) and K₂CO₃ (31 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (40 mg, 77%).

¹H NMR (400 MHz, CDCl₃) 1.31 (3H, t, J=7 Hz), 2.30 (3H, s), 4.30 (2H, q, J=7 Hz), 4.61 (2H, s), 6.13 (1H, d, J=3.5 Hz), 6.31 (1H, d, J=3.5 Hz), 6.57 (1H, d, J=9 Hz), 6.92-7.09 (5H, m), 7.29 (1H, d, J=2.5 Hz), 7.37 (1H, broad s), 7.65 (1H, dd, J=1.5, 5 Hz), 8.50 (1H, d, J=5 Hz).

LC/MS t=4.03 min [MH+] 465/467.

### b) 2-{2-[5-Chloro-2-(4-fluoro-benzyloxy)-phenyl]-5-methyl-pyrrol-1-yl}-isonicotinic acid

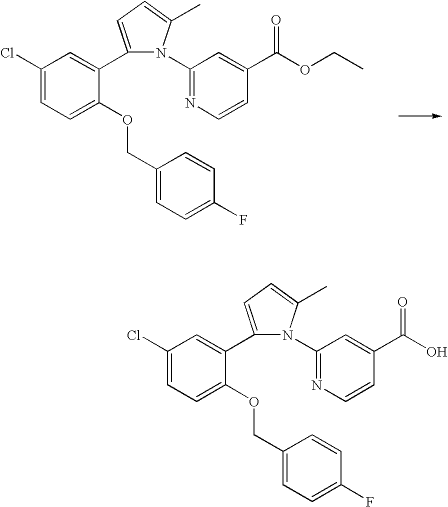

2-{2-[5-Chloro-2-(4-fluoro-benzyloxy)-phenyl]-5-methyl-pyrrol-1-yl}-isonicotinic acid ethyl ester (40 mg, 0.09 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (33 mg, 88%).

¹H NMR (400 MHz, d6-DMSO) 2.20 (3H, s), 4.70 (2H, s), 6.09 (1H, broad d, J=3.5 Hz), 6.27 (1H, d, J=3.5 Hz), 6.87 (1H, d, J=9 Hz), 7.10-7.14 (5H, m), 7.21 (1H, dd, J=3, 9 Hz), 7.30 (1H, broad s), 7.70 (1H, dd, J=3.5, 5 Hz), 8.56 (1H, d, J=5 Hz), 13.70 (1H, broad s).

LC/MS t=4.03 min [MH+] 437/439.

### Example 127

### 2-{2-[5-Chloro-2-(2-chloro-4-fluoro-benzyloxy)-phenyl]-5-methyl-pyrrol-1-yl}-isonicotinic acid

### a) 2-{2-[5-Chloro-2-(2-chloro-4-fluoro-benzyloxy)-phenyl]-5-methyl-pyrrol-1-yl}-isonicotinic acid ethyl ester

2-Chloro-4-fluoro-benzyl bromide (25 mg, 0.11 mmol) was added to 2-{2-[5-chloro-2-(hydroxy)-phenyl]-5-methyl-pyrrol-1-yl}-isonicotinic acid ethyl ester (40 mg, 0.11 mmol) and K₂CO₃ (31 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (46 mg, 82%).

¹H NMR (400 MHz, CDCl₃) 1.31 (3H, t, J=7 Hz), 2.31 (3H, s), 4.30 (2H, q, J=7 Hz), 4.68 (2H, s), 6.14 (1H, dd, J=1, 3.5 Hz), 6.33 (1H, d, J=3.5 Hz), 6.57 (1H, d, J=9 Hz), 6.90 (1H, ddd, J=2.5, 9 Hz), 7.03-7.11 (3H, m), 7.30 (1H, d, J=2.5 Hz), 7.40 (1H, broad s), 7.67 (1H, dd, J=1.5, 5 Hz), 8.50 (1H, d, J=5 Hz).

LC/MS t=4.20 min [MH+] 499/501/503

### b) 2-{2-[5-Chloro-2-(2-chloro-4-fluoro-benzyloxy)-phenyl]-5-methyl-pyrrol-1-yl}-isonicotinic acid

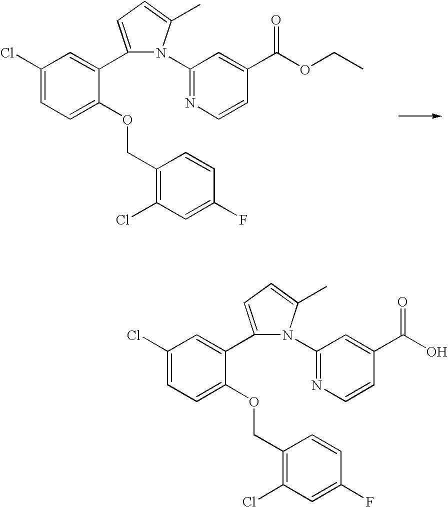

2-{2-[5-Chloro-2-(2-chloro-4-fluoro-benzyloxy)-phenyl]-5-methyl-pyrrol-1-yl}-isonicotinic acid ethyl ester (46 mg, 0.09 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (40 mg, 92%).

¹H NMR (400 MHz, d6-DMSO) 2.19 (3H, s), 4.73 (2H, s), 6.09 (1H, d, J=3.5 Hz), 6.29 (1H, d, J=3.5 Hz), 6.92 (1H, d, J=9 Hz), 7.11 (1H, d, J=2.5 Hz), 7.11-7.25 (3H, m), 7.29 (1H, s), 7.44 (1H, dd, J=2.5, 9 Hz), 7.70 (1H, dd, J=1.5, 5 Hz), 8.53 (1H, d, J=5 Hz), 13.60 (1H, broad s).

LC/MS t=4.28 min [MH−] 469/471/473.

### Example 128

### 2-{2-[5-Chloro-2-(2,4-difluoro-benzyloxy)-phenyl]-5-methyl-pyrrol-1-yl}-isonicotinic acid

### a) 2-{2-[5-Chloro-2-(2,4-difluoro-benzyloxy)-phenyl]-5-methyl-pyrrol-1-yl}-isonicotinic acid ethyl ester

2,4-Difluoro-benzyl bromide (0.015 ml, 0.1 1 mmol) was added to 2-{2-[5-chloro-2-(hydroxy)-phenyl]-5-methyl-pyrrol-1-yl}-isonicotinic acid ethyl ester (40 mg, 0.1 1 mmol) and K₂CO₃ (31 mg, 0.31 mmol) in DMF (1 ml) and the reaction mixture was heated at 60° C. for 16 hours. Upon cooling the reaction mixture was diluted with EtOAc, washed with water, brine, dried (MgSO₄), filtered and concentrated. The residue was purified by chromatography on silica gel with isohexane/EtOAc (10%) as eluant, to give the title compound (44 mg, 81%).

¹H NMR (400 MHz, CDCl₃) 1.32 (3H, t, J=7 Hz), 2.30 (3H, s), 4.30 (2H, q, J=7 Hz), 4.67 (2H, s), 6.12 (1H, dd, J=1, 3.5 Hz), 6.31 (1H, d, J=3.5 Hz), 6.61 (1H, d, J=9 Hz), 6.71-6.83 (2H, m), 7.02-7.11 (2H, m), 7.27 (1H, d, J=2.5 Hz), 7.39 (1H, s), 7.66 (1H, dd, J=1.5, 5 Hz), 8.50 (1H, d, J=5 Hz).

LC/MS t=4.08 min [MH+] 483/485

### b) 2-{2-[5-Chloro-2-(2,4-difluoro-benzyloxy)-phenyl]-5-methyl-pyrrol-1-yl}-isonicotinic acid

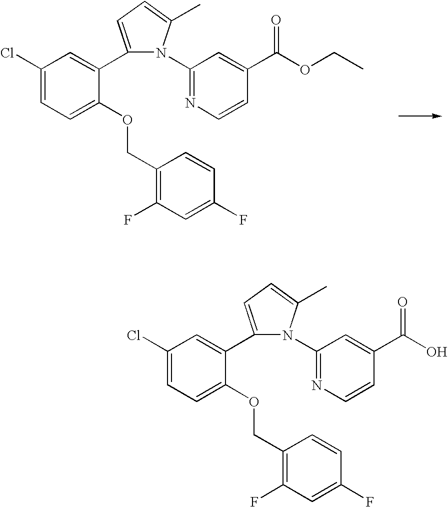

2-{2-[5-Chloro-2-(2,4-difluoro-benzyloxy)-phenyl]-5-methyl-pyrrol-1-yl}-isonicotinic acid ethyl ester (44 mg, 0.09 mmol) was heated at reflux in a 1:1 mixture of ethanol/2M NaOH (2 ml) in a sealed vessel for 1.5 hours. Upon cooling, the reaction was diluted with ethyl acetate, washed with 2M HCl and brine, dried (MgSO₄) and concentrated to give the title compound (38 mg, 92%).

¹H NMR (400 MHz, d6DMSO) 2.17 (3H, s), 4.72 (2H, s), 6.07 (1H, broad d, J=3.5 Hz), 6.26 (1H, d, J=3.5 Hz), 6.96 (1H, d, J=9 Hz), 7.03 (1H, ddd, J=2.5, 9 Hz), 7.10 (1H, d, J=2.5 Hz), 7.12-7.26 (3H, m), 7.27 (1H, s), 7.69 (1H, dd, J=1.5, 5 Hz), 8.53 (1H, d, J=5 Hz), 13.60 (1H, broad s).

LC/MS t=4.06 min [MH+] 455/457

### Example 129

### 5-{2-]2-(Benzyloxy)-phenyl]-5-methyl-pyrrol-1-yl}-nicotinic acid

### a) 5-{2-[2-Benzyloxy)-phenyl]-5-methyl-pyrrol-1-yl}-nicotinic acid ethyl ester

5-{2-[2-(Hydroxy)-phenyl]-5-methyl-pyrrol-1-yl}-nicotinic acid ethyl ester (0.11 6g, 0.358 mmol), benzyl bromide (0.064 ml, 0.537 mmol) and potassium carbonate (0.099g, 0.716 mmol) were heated in DMF (1 ml) at 65° C. in a nitrogen atmosphere for 3 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a yellow oil (0.041 g, 28%).

¹H-NMR (400 MHz, CDCl₃) 1.32 (3H, t, J=7 Hz), 2.17 (3H, s), 4.31 (2H, q, J=7 Hz), 4.75 (2H, s), 6.17 (1H, dd, J=1 Hz, 3 Hz), 6.32 (1H, d, J=3 Hz), 6.67 (1H, d, 9 Hz), 6.91 (1H, ddd, J=1Hz, 7 Hz), 7.07 (2H, dd, J=2 Hz, 9 Hz), 7.14 (1H, ddd, J=2 Hz, 7 Hz), 7.28-7.32 (4H, m), 7.90 (1H, t, J=2 Hz), 8.37 (1H, d, J=2 Hz), 9.02 (1H, d, J=2 Hz).

LC/MS t=3.78 min [MH⁺] 413

### b) 5-{2-[2-(Benzyloxy)-phenyl]-5-methyl-pyrrol-1-yl}-nicotinic acid

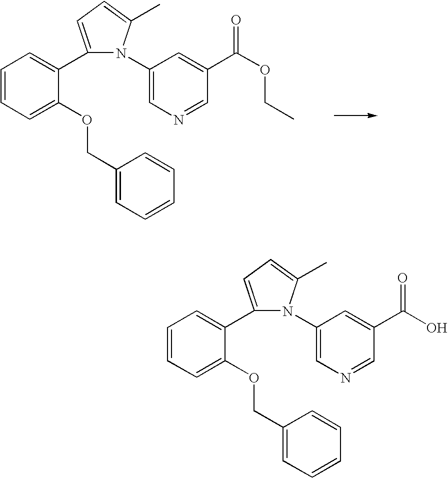

5-{2-[2-(Benzyloxy)-phenyl]-5-methyl-pyrrol-1-yl}-nicotinic acid ethyl ester (0.041 g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an orange solid (0.038g, 99%).

¹H-NMR (400 MHz, CDCl₃) 2.17 (3H, s), 4.74 (2H, s), 6.18 (1H, d, J=3 Hz), 6.32 (1H, d, J=3 Hz), 6.68 (1H, d, J=9 Hz), 6.91 (1H, t, J=8 Hz), 7.08 (2H, broad d, J=8 Hz), 7.13 (1H, ddd, J=2 Hz, 8 Hz), 7.21-7.30 (4H, m), 7.98 (1H, t, J=2 Hz), 8.37 (1H, d, J=2 Hz), 9.07 (1H, d, J=2 Hz).

LC/MS t=3.70 min [MH⁺] 385

### Example 130

### 5-{2-[2-(2-Chloro-4-fluoro-benzyloxy)-phenyl]-5-methyl-pyrrol-1-yl]-nicotinic acid

### a) 5-{2-[2-(2-Chloro-4-fluoro-benzyloxy)-phenyl]-5-methyl-pyrrol-1-yl}-nicotinic acid ethyl ester

5-{2-[2-(Hydroxy)-phenyl]-5-methyl-pyrrol-1-yl}-nicotinic acid ethyl ester (0.11 6g, 0.358 mmol), 2-chloro-4-fluorobenzyl bromide (0.120g, 0.537 mmol) and potassium carbonate (0.099g, 0.716 mmol) were heated in DMF (1 ml) at 65° C. in a nitrogen atmosphere for 3 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a yellow oil (0.035g, 30%).

¹H-NMR (400 MHz, CDCl₃) 1.33 (3H, t, J=7 Hz), 2.18 (3H, s), 4.32 (2H, q, J=7 Hz), 4.78 (2H, s), 6.18 (1H, d, J=3 Hz), 6.32 (1H, d, J=3 Hz), 6.65 (1H, d, 9 Hz), 6.89-7.01 (3H, m), 7.07-7.18 (2H, m), 7.29 (1H, d, J=2 Hz), 7.93 (1H, t, J=2 Hz), 8.40 (1H, d, J=2 Hz), 9.03 (1H, d, J=2 Hz).

LC/MS t=3.96 min [MH⁺] 465

### b) 5-{2-[2-(2-Chloro-4-fluoro-benzyloxy)-phenyl]-5-methyl-pyrrol-1-yl}-nicotinic acid

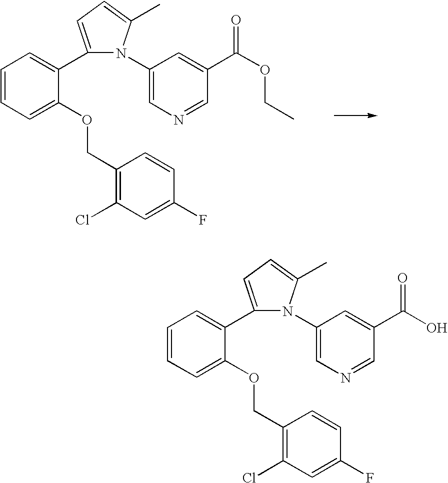

5-{2-[2-(2-Chloro-4-fluoro-benzyloxy)-phenyl]-5-methyl-pyrrol-1-yl}-nicotinic acid ethyl ester (0.035g), 2M NaOH (1 ml) and EtOH (2 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as a yellow solid (0.029g, 89%).

¹H-NMR (400 MHz, MeOD) 2.15 (3H, s), 4.75 (2H, s), 6.13 (1H, d, J=3 Hz), 6.21 (1H, d, J=3 Hz), 6.80 (1H, d, J=9 Hz),6.98 (1H, t, J=8 Hz), 7.03 (1H, ddd, J=2 Hz, 8 Hz), 7.08-7.13 (1H, m), 7.17-7.24 (2H, m), 7.32 (1H, dd, J=1 Hz, 7 Hz), 7.91 (1H, t, J=1 Hz), 8.22 (1H, d, J=1 Hz), 8.91 (1H, broad s).

LC/MS t=3.96 min [MH⁺] 437.

### Example 131

### 5-{2-[5-Bromo-2-(benzyloxy)-phenyl]-5-methyl-pyrrol-1-yl}-nicotinic acid

### a) 5-{2-[5-Bromo-2-(hydroxy)-phenyl]-5-methyl-pyrrol-1-yl}-nicotinic acid ethyl ester

5-{2-[5-Bromo-2-(4-methoxy-benzyloxy)-phenyl]-5-methyl-pyrrol-1-yl}-nicotinic acid ethyl ester (1.7g, 3.26 mmol) was stirred at room temperature and under nitrogen in 4.0M hydrogen chloride in dioxane (15 ml) for 20 minutes. The solvent was then removed in vacuo and the residue diluted with EtOAc. The solution was then washed with saturated NaHCO₃ and brine, dried over MgSO₄, filtered and concentrated in vacuo. The resultant oil was purified by chromatography on silica gel eluting with 30% EtOAc/iso-hexane. This yielded the title compound as a yellow oil (0.361g, 27%).

¹H-NMR (400 MHz, CDCl₃) 1.40 (3H, t, J=7 Hz), 2.18 (3H, s), 4.41 (2H, q, J=7 Hz), 5.83 (1H, s), 6.20 (1H, d, J=3 Hz), 6.38 (1H, d, J=3 Hz), 6.72 (1H, d, J=9 Hz), 6.95 (1H, d, 3 Hz), 7.19 (1H, dd, J=3 Hz, 9 Hz), 8.05 (1H, t, J=2 Hz), 8.49 (1H, d, J=3 Hz), 9.13 (1H, d, J=2 Hz).

LC/MS t=3.51 min [MH⁺] 401/403.

### b) 5-{2-[6-Bromo-2-(benzyloxy)-phenyl]-5-methyl-pyrrol-1-yl}-nicotinic acid ethyl ester

5-{2-[5-Bromo-2-(hydroxy)-phenyl]-5-methyl-pyrrol-1-yl}-nicotinic acid ethyl ester (0.06g, 0.125 mmol), benzyl bromide (0.022 ml, 0.187 mmol) and potassium carbonate (0.034g, 0.249 mmol) were heated in DMF (1 ml) at 65° C. in a nitrogen atmosphere for 2.5 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a yellow oil (0.057g, 77%).

¹H-NMR (400 MHz, CDCl₃) 1.34 (3H, t, J=7 Hz), 2.15 (3H, s), 4.32 (2H, q, J=7 Hz), 4.69 (2H, s), 6.17 (1H, d, J=3 Hz), 6.32 (1H, d, J=3 Hz), 6.53 (1H, d, 9 Hz), 7.02 (2H, m), 7.22 (1H, dd, J=3 Hz, 9 Hz), 7.29 (3H, m), 7.45 (1H, d, J=3 Hz), 7.89 (1H, t, J=2 Hz), 8.36 (1H, d, J=2 Hz), 9.05 (1H, d, J=2 Hz).

LC/MS t=3.99 min [MH⁺] 491/493.

### c) 5-{2-[5-Bromo-2-benzyloxy)-phenyl]-5-methyl-pyrrol-1-yl}-nicotinic acid

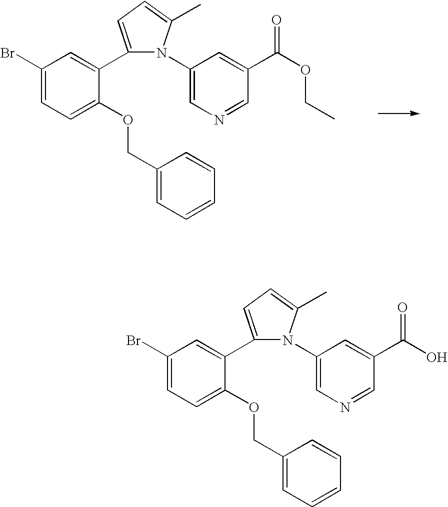

5-{2-[5-Bromo-2-(benzyloxy)-phenyl]-5-methyl-pyrrol-1-yl}nicotinic acid ethyl ester (0.057g), 2M NaOH (1.5 ml) and EtOH (2.5 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an off white solid (0.053g, 99%).

¹H-NMR (400 MHz, MeOD) 2.13 (3H, s), 4.70 (2H, s), 6.14 (1H, d, J=3 Hz), 6.26 (1H, d, J=3 Hz), 6.73 (1H, d, 9 Hz), 7.08 (2H, dd, J=1 Hz, 8 Hz), 7.25-7.32 (4H, m), 7.44 (1H, d, J=2 Hz), 7.91 (1H, t, J=2 Hz), 8.23 (1H, d, J=2 Hz), 8.93 (1H, d, J=1 Hz).

LC/MS t=4.00 min [MH⁺] 463/465.

### Example 132

### 5-{2-[5-Bromo-2-(4-chloro-benzyloxy)-phenyl]-5-methyl-pyrrol-1-yl}-nicotinic acid

### a) 5-{2-[5-Bromo-2-(4-chloro-benzyloxy)-phenyl]-5-methyl-pyrrol-1-yl}-nicotinic acid ethyl ester

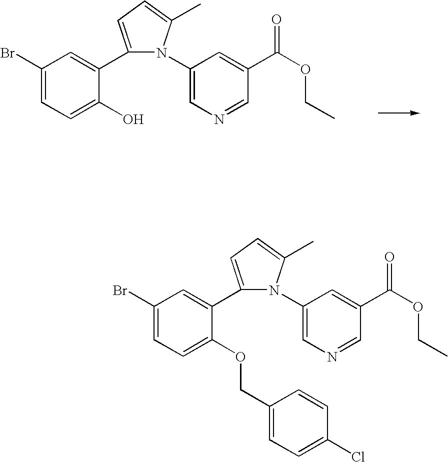

5-{2-[5-Bromo-2-(hydroxy)-phenyl]-5-methyl-pyrrol-1-yl}-nicotinic acid ethyl ester (0.06g, 0.125 mmol), 4-chlorobenzyl bromide (0.038g, 0.187 mmol) and potassium carbonate (0.034g, 0.249 mmol) were heated in DMF (1 ml) at 65° C. in a nitrogen atmosphere for 2.5 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a yellow oil (0.080g, 100%).

¹H-NMR (400 MHz, CDCl₃) 1.35 (3H, t, J=7 Hz), 2.16 (3H, s), 4.34 (2H, q, J=7 Hz), 4.65 (2H, s), 6.17 (1H, d, J=3 Hz), 6.30 (1H, d, J=3 Hz), 6.50 (1H, d, 9 Hz), 6.98 (2H, d, J=9 Hz), 7.23 (1H, d, J=3 Hz), 7.25-7.29 (2H, m), 7.46 (1H, d, J=3 Hz), 7.87 (1H, t, J=2 Hz), 8.36 (1H, d, J=2 Hz), 9.04 (1H, d, J=2 Hz).

LC/MS t=4.13 min [MH⁺] 527.

### b) 5-{2-[5-Bromo-2-(4-chloro-benzyloxy)-phenyl]-5-methyl-pyrrol-1-yl}-nicotinic acid

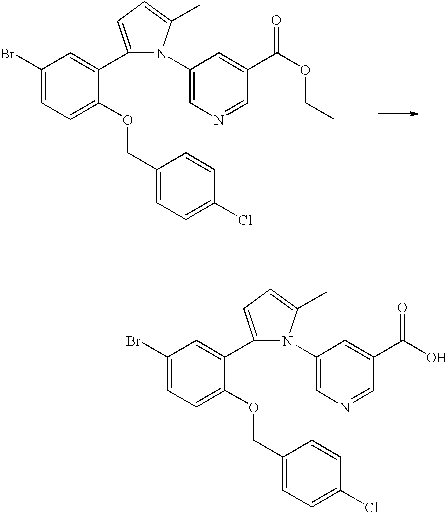

5-{2-[5-Bromo-2-(4-chloro-benzyloxy)-phenyl]-5-methyl-pyrrol-1-yl}-nicotinic acid ethyl ester (0.080g), 2M NaOH (1.5 ml) and EtOH (2.5 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an off white solid (0.056g, 73%).

¹H-NMR (400 MHz, MeOD) 2.13 (3H, s) 4.71 (2H, s), 6.12 (1H, d, J=3 Hz), 6.25 (1H, d, J=3 Hz), 6.67 (1H, d, J=9 Hz), 7.07 (2H, d, J=8 Hz), 7.25 (1H, dd, J=3 Hz, 9 Hz), 7.30 (2H, d, J=8 Hz), 7.38 (1H, d, J=3 Hz), 7.97 (1H, t, J=2 Hz), 8.17 (1H, d, J=2 Hz), 8.97 (1H, d, J=2 Hz).

LC/MS t=4.22 min [MH⁺] 497/499.

### Example 133

### 5-{2-[5-Bromo-2-(4-fluoro-benzyloxy)-phenyl]-5-methyl-pyrrol-1-yl}-nicotinic acid

### a) 52-[5-Bromo-2-(4-fluoro-benzyloxy)-phenyl]-5-methyl-pyrrol-1-yl}-nicotinic acid ethyl ester

5-{2-[5-Bromo-2-(hydroxy)-phenyl]-5-methyl-pyrrol-1-yl}-nicotinic acid ethyl ester (0.06g, 0.125 mmol), 4-fluoro-benzyl bromide (0.024 ml, 0.187 mmol) and potassium carbonate (0.034g, 0.249 mmol) were heated in DMF (1 ml) at 65° C. in a nitrogen atmosphere for 2.5 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a yellow oil (0.051g, 67%).

¹H-NMR (400 MHz, CDCl₃) 1.35 (3H, t, J=7 Hz), 2.15 (3H, s), 4.33 (2H, q, J=7 Hz), 4.63 (2H, s), 6.17 (1H, d, J=3 Hz), 6.30 (1H, d, J=3 Hz), 6.52 (1H, d, 9 Hz), 6.95-7.04 (4H, m), 7.23 (1H, d, J=2 Hz), 7.46 (1H, d, J=2 Hz), 7.86 (1H, t, J=2 Hz), 8.34 (1H, d, J=2 Hz), 9.04 (1H, d, J=2 Hz).

LC/MS t=4.00 min [MH⁺] 509/511

### b) 52-[5-Bromo-2-(4-fluoro-benzyloxy)-phenyl]-5-methyl-pyrrol-1-yl}-nicotinic acid

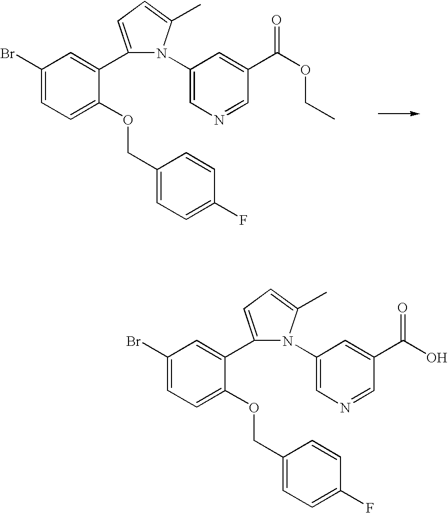

5-{2-[5-Bromo-2-(4-fluoro-benzyloxy)-phenyl]-5-methyl-pyrrol-1-yl}-nicotinic acid ethyl ester (0.051 g), 2M NaOH (1.5 ml) and EtOH (2.5 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an off white solid (0.043g, 89%).

¹H-NMR (400 MHz, MeOD) 2.12 (3H, s), 4.69 (2H, s), 6.11 (1H, d, J=3 Hz), 6.23 (1H, d, J=3 Hz), 6.68 (1H, d, J=9 Hz), 7.00-7.07 (2H, m), 7.09-7.15 (2H, m), 7.24 (1H, dd, J=3 Hz, 9 Hz), 7.38 (1H, d, J=2 Hz), 7.97 (1H, t, J=1 Hz), 8.14 (1H, d, J=2 Hz), 8.95 (1H, broad s).

LC/MS t=4.00 min [MH⁺] 481/483

### Example 134

### 5-{2-[5-Bromo-2-3,4-dichloro-benzyloxy)-phenyl]-5-methyl-pyrrol-1-yl}-nicotinic acid

### a) 5-{2-[5-Bromo-2-(3,4-dichloro-benzyloxy)-phenyl]-5-methyl-pyrrol-1-yl}-nicotinic acid ethyl ester

5-{2-[5-Bromo-2-(hydroxy)-phenyl]-5-methyl-pyrrol-1-yl}-nicotinic acid ethyl ester (0.06g, 0.125 mmol), 3,4-dichlorobenzyl bromide (0.032 ml, 0.187 mmol) and potassium carbonate (0.034g, 0.249 mmol) were heated in DMF (1 ml) at 65° C. in a nitrogen atmosphere for 2.5 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a clear oil (0.094g, 100%).

¹H-NMR (400 MHz, CDCl₃) 1.35 (3H, t, J=7 Hz), 2.18 (3H, s), 4.33 (2H, q, J=7 Hz), 4.64 (2H, s), 6.19 (1H, d, J=3 Hz), 6.31 (1H, d, J=3 Hz), 6.49 (1H, d, 9 Hz), 6.88 (1H, dd, J=1 Hz, 9 Hz), 7.10 (1H, d, J=1 Hz), 7.24 (1H, d, J=3 Hz), 7.38 (1H, d, J=9 Hz), 7.48 (1H, d, J=3 Hz), 7.90 (1H, t, J=2 Hz), 8.39 (1H, d, J=2 Hz) 9.0 (1H, d, J=2 Hz).

LC/MS t=4.25 min [MH⁺] 561

### b) 5-{2-[5-Bromo-2-(3,4-dichloro-benzyloxy)-phenyl]-5-methyl-pyrrol-1-yl}-nicotinic acid

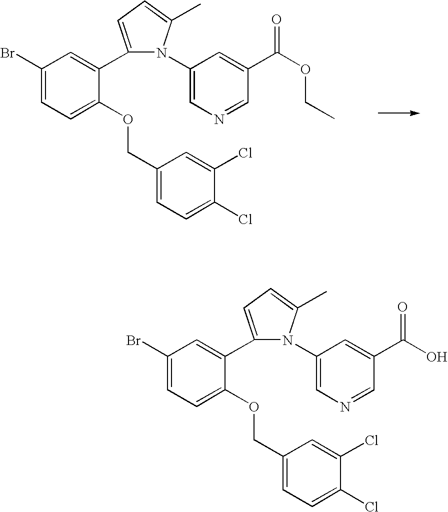

5-{2-[5-Bromo-2-(3,4-dichloro-benzyloxy)-phenyl]-5-methyl-pyrrol-1-yl}-nicotinic acid ethyl ester (0.094g), 2M NaOH (1.5 ml) and EtOH (2.5 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an off white solid (0.091 g, 100%).

¹H-NMR (400 MHz, CDCl₃) 2.12 (3H, s), 4.66 (2H, s), 6.16 (1H, d, J=3 Hz), 6.31 (1H, d, J=3 Hz), 6.43 (1H, d, 9 Hz), 6.88 (1H, dd, J=2 Hz, 9 Hz), 7.13-7.18 (2H, m), 7.35-7.39 (2H, m), 7.90 (1H, t, J=2 Hz), 8.28 (1H, d, J=2 Hz) 9.04 (1H, broad s).

LC/MS t=4.41 min [MH⁺] 533.

### Example 135

### 5-{2-[5-Bromo-2-(2-chloro-4-fluoro-benzyloxy)-phenyl]-5-methyl-pyrrol-1-yl}-nicotinic acid

### a) 5-{2-[5-Bromo-2-(2-chloro-4-fluoro-benzyloxy)-phenyl]-5-methyl-pyrrol-1-yl}-nicotinic acid ethyl ester

5-{2-(5-Bromo-2-hydroxy-phenyl}-5methyl-pyrrol-1-yl]-nicotinic acid ethyl ester (0.06g, 0.125 mmol), 2-chloro-4-fluoro-benzyl bromide (0.042g, 0.187 mmol) and potassium carbonate (0.034g, 0.249 mmol) were heated in DMF (1 ml) at 65° C. in a nitrogen atmosphere for 2.5 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a clear oil (0.079g, 98%).

¹H-NMR (400 MHz, CDCl₃) 1.35 (3H, t, J=7 Hz), 2.17 (3H, s), 4.34 (2H, q, J=7 Hz), 4.72 (2H, s), 6.18 (1H, d, J=3 Hz), 6.31 (1H, d, J=3 Hz), 6.51 (1H, d, 9 Hz), 6.91 (2H, dd, J=1 Hz, 7 Hz), 7.09 (1H, dt, J=1 Hz, 9 Hz), 7.25 (1H, d, J=3 Hz), 7.45 (1H, d, J=3 Hz), 7.91 (1H, t, J=2 Hz), 8.40 (1H, d, J=3 Hz), 9.06 (1H, d, J=2 Hz).

LC/MS t=4.14 min [MH⁺] 545

### b) 5-{2-[5-Bromo-2-(2-chloro-4-fluoro-benzyloxy)-phenyl]-5-methyl-pyrrol-1-yl}-nicotinic acid

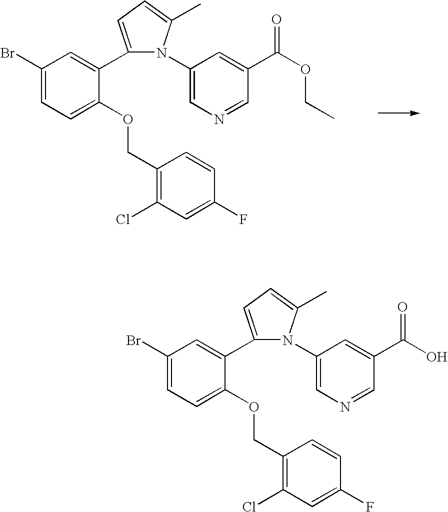

5-{2-[5-Bromo-2-(2-chloro-4-fluoro-benzyloxy)-phenyl]-5-methyl-pyrrol-1-yl}-nicotinic acid ethyl ester (0.079g), 2M NaOH (1.5 ml) and EtOH (2.5 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an off white solid (0.057g, 76%).

¹H-NMR (400 MHz, MeOD) 2.15 (3H, s), 4.78 (2H, s), 6.12 (1H, d, J=3 Hz), 6.24 (1H, d, J=3 Hz), 6.69 (1H, d, J=9 Hz), 7.00-7.06 (2H, m), 7.20 (1H, broad d, J=9 Hz), 7.27 (1H, dd, J=3 Hz; 9 Hz), 7.39 (1H, d, J=3 Hz), 7.99 (1H, t, J=2 Hz), 8.20 (1H, d, J=2 Hz), 8.98 (1H, broad s).

LC/MS t=4.22 min [MH⁺] 517.

### Example 136

### 5-{2-[5-Bromo-2-(2,4-difluoro-benzyloxy)-phenyl]-5-methyl-pyrrol-1-yl}-nicotinic acid

### a) 5-{2-[5-Bromo-2-(2,4-difluoro-benzyloxy)-phenyl]-5-methyl-pyrrol-1-yl}-nicotinic acid ethyl ester

5-{2-[5-Bromo-2-(hydroxy)-phenyl]-5-methyl-pyrrol-1-yl}-nicotinic acid ethyl ester (0.06g, 0.125 mmol), 2,4-difluorobenzyl bromide (0.024 ml, 0.187 mmol) and potassium carbonate (0.034g, 0.249 mmol) were heated in DMF (1 ml) at 65° C. in a nitrogen atmosphere for 2.5 hours. Upon cooling the mixture was diluted with EtOAc and water. The organic layer was extracted and the aqueous layer washed with 3×EtOAc. The combined organics were then washed with brine and concentrated in vacuo. The residue was purified by chromatography on silica gel eluting with 15% EtOAc/iso-hexane. This yielded the title compound as a yellow oil (0.073g, 92%).

¹H-NMR (400 MHz, CDCl₃) 1.37 (3H, t, J=7 Hz), 2.16 (3H, s), 4.36 (2H, q, J=7 Hz), 4.70 (2H, s), 6.16 (1H, d, J=3 Hz), 6.29 (1H, d, J=3 Hz), 6.57 (1H, d, 9 Hz), 6.76-6.83 (2H, m), 6.93-7.00 (1H, m), 7.26-7.28 (1H, m), 7.43 (1H, d, J=2 Hz), 7.90 (1H, d, J=2 Hz), 8.37 (1H, d, J=2 Hz), 9.06 (1H, d, J=2 Hz).

LC/MS t=4.01 min [MH⁺] 527/529.

### b) 5-{2-[5-Bromo-2-(2,4-difluoro-benzyloxy)-phenyl]-5-methyl-pyrrol-1-yl}-nicotinic acid

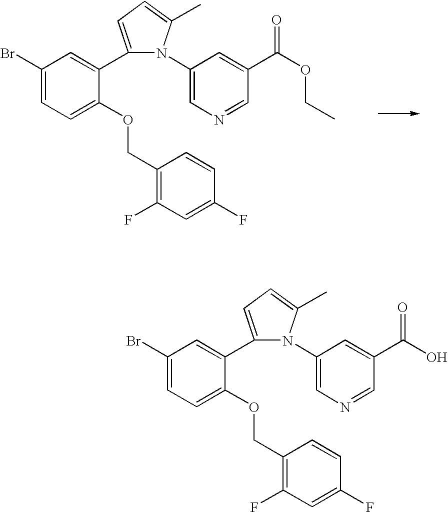

5-{2-[5-Bromo-2-(2,4-difluoro-benzyloxy)-phenyl]-5-methyl-pyrrol-1-yl}-nicotinic acid ethyl ester (0.073g), 2M NaOH (1.5 ml) and EtOH (2.5 ml) were heated at 100° C. in a sealed vessel for 1 hour. Upon cooling the mixture was diluted in EtOAc and washed with 0.5M citric acid. The organic layer was extracted and the aqueous layer washed with 3×EtOAc. The combined organic extracts were washed with brine and dried over MgSO₄, filtered and concentrated by nitrogen blowdown, to yield the title compound as an off white solid (0.063g, 92%).

¹H-NMR (400 MHz, MeOD) 2.12 (3H, s), 4.75 (2H, s), 6.10 (1H, broad s), 6.21 (1H, d, J=3 Hz), 6.73 (1H, d, J=9 Hz), 6.88-6.97 (2H, m), 7.10 (1H, q, J=7 Hz), 7.28 (1H, dd, J=2 Hz, 9 Hz), 7.35 (1H, broad s), 7.98 (1H, broad s), 8.15 (1H, broad s), 8.96 (1H, broad s).

LC/MS t=4.00 min [MH⁺] 499/501.

### Example 137

### 3-{2-[6-Bromo-2-(biphenyl-4-ylmethoxy)-phenyl]-5-methyl-pyrrol-1-yl}-benzoic acid

### a) 3-{2-[5-Bromo-2-(biphenyl-4-ylmethoxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (0.07g, 0.25 mmol), 4-bromomethyl-biphenyl (0.074g, 0.3 mmol), potassium carbonate (0.069g, 0.5 mmol) and DMF (1 ml) were stirred under nitrogen at 50° C. for 4h. The solvent was then removed by heating under high vacuum. The residue was taken up in DCM, washed with H₂O and the organics separated using a phase separator column, and the solvent removed in vacuo. The residue was then purified by column chromatography on a SPE column (5 g) eluting in 10% EtOAc/i-hexane. This yielded the title compound as a clear oil (0.074g, 74%).

LC/MS t=4.45 min [MH⁺] 566/568.

### b) 3-{2-[5-Bromo-2-(biphenylylmethoxy)-phenyl]-5-methyl-pyrrol-1-yl}-benzoic acid

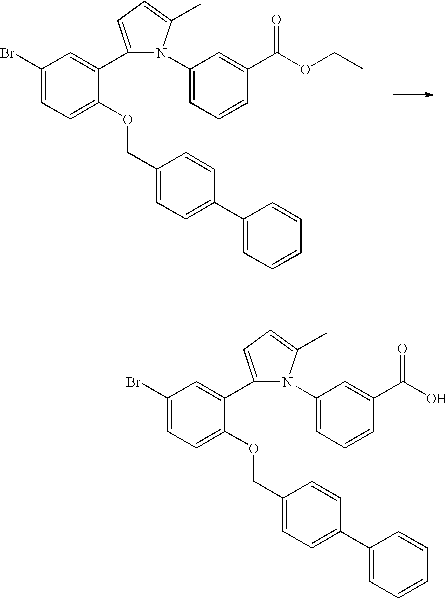

Procedure as for 3-{2-[5-bromo-2-(2,4,6-trifluoro-benzyloxy)-phenyl]-5-methyl-pyrrol-1-yl}-benzoic acid.

LC/MS t=4.32 min [MH⁺] 538/540.

### Example 138

### 3-{2-[5-Bromo-2-(2-bromo-4-fluoro-benzyloxy)-phenyl]-5-methyl-pyrrol-1-yl)--benzoic acid

### a) 3-{2-[5-Bromo-2-(2-bromo-4-fluoro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.39 min [MH⁺] 586/588.

### b) 3-{2-[5-Bromo-2-(2-bromo-4-fluoro-benzyloxy)-phenyl]-5-methyl-pyrrol-1-yl}-benzoic acid

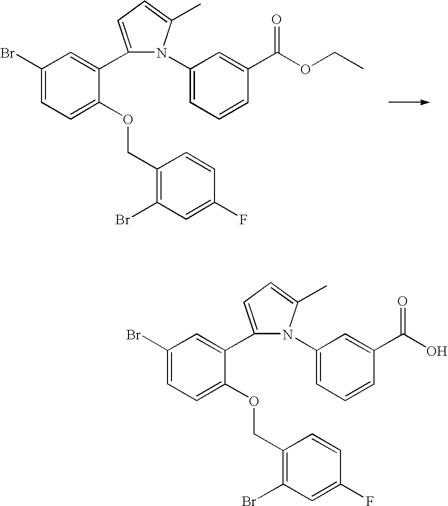

Procedure as for 3-{2-[5-bromo-2-(2,4,6-trifluoro-benzyloxy)-phenyl]-5-methyl-pyrrol-1-yl}-benzoic acid.

¹H-NMR (400 MHz, CDCl₃) 2.04 (3H, s), 4.69 (2H, s), 6.07 (1H, d, J=3 Hz), 6.29 (1H, d, J=3 Hz), 6.42 (1H, d, J=9 Hz), 6.85-6.95 (2H, m), 7.02-7.10 (3H, m), 7.18 (1H, dd, J=2 Hz, 8 Hz), 7.25-7.28 (1H, m excess), 7.72 (1H, s), 7.81 (1H, d, J=8 Hz).

LC/MS t=4.26 min [MH⁺] 558/560.

### Example 139

### 3-{2-[5-Bromo-2-(3-chloro-4-fluoro-benzyloxy)-phenyl]-5-methyl-pyrrol-1-yl-benzoic acid

### a) 3-{2-[5-Bromo-2-(3-chloro-4-fluoro-benzyloxy)-phenyl]-5-methyl-pyrrol-1-yl}-benzoic acid ethyl ester

Procedure as for 3-{2-[5-bromo-2-(biphenylylmethoxy)-phenyl]-5-methyl-pyrrol-1-yl}-benzoic acid ethyl ester using the appropriate benzyl bromide.

LC/MS t=4.34 min [MH⁺] 542/544.

### b) 3-{2-[5-Bromo-2-(3-chloro-4-fluoro-benzyloxy)-phenyl]-5-methyl-pyrrol-1-yl}-benzoic acid

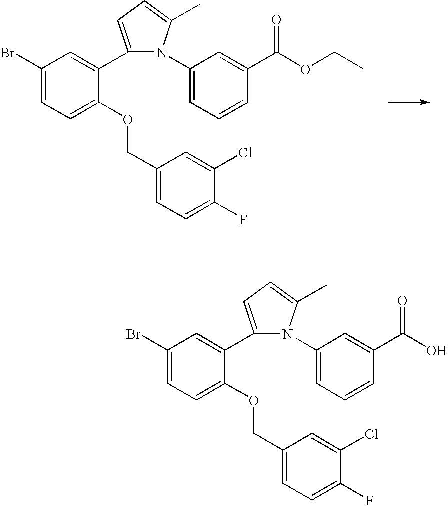

Procedure as for 3-{2-[5-bromo-2-(2,4,6trifluoro-benzyloxy)-phenyl]-5-methyl-pyrrol-1-yl}-benzoic acid.

LC/MS t=19 min [MH⁺] 514/516.

### Example 140

### 3-{2-[5-Bromo-2-(4-bromo-2-fluoro-benzyloxy)-phenyl]-5-methyl-pyrrol-1-yl}-benzoic acid

### a) 3-{2-[5-Bromo-2-(4-bromo-2-fluoro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.39 min [MH⁺] 586/588.

### b) 3-{2-[5-Bromo-2-(4-bromo-2-fluoro-benzyloxy)-phenyl]-5-methyl-pyrrol-1-yl}-benzoic acid

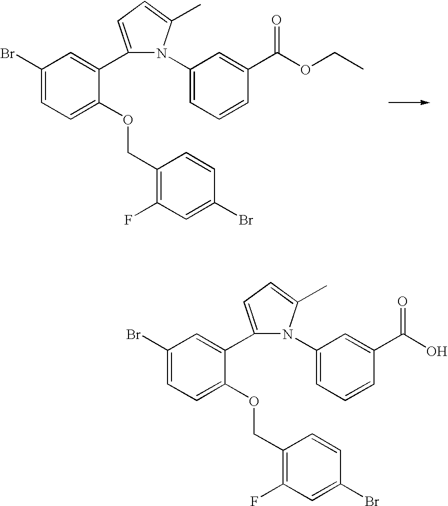

Procedure as for 3-{2-[5-bromo-2-(2,4,6-trifluoro-benzyloxy)-phenyl]-5-methyl-pyrrol-1-yl}-benzoic acid.

¹H-NMR (400 MHz, CDCl₃) 2.10 (3H, s), 4.70 (2H, s), 6.10 (1H, d, J=3 Hz), 6.28 (1H, d, J=3 Hz), 6.47 (1H, d, J=9 Hz), 6.87 (1H, t, J=8 Hz), 7.07-7.20 (4H, m), 7.25-7.28 (1H, m excess), 7.31 (1H, d, J=2 Hz), 7.73 (1H, t, J=1 Hz), 7.87 (1H, dt, J=1 Hz, 8 Hz).

LC/MS-t=4.25 min [MH⁺] 558/560.

### Example 141

### 3-{2-[5-Bromo-2-(3,4-difluoro-benzyloxy)-phenyl]-5-methyl-pyrrol-1-yl}-benzoic acid

### a) 3-{2-[5-Bromo-2-(3,4-difluoro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.25 min [MH⁺] 526/528.

### b) 3-{2-[5-Bromo-2-(3,4-difluoro-benzyloxy)-phenyl]-5-methyl-pyrrol-1-yl}-benzoic acid

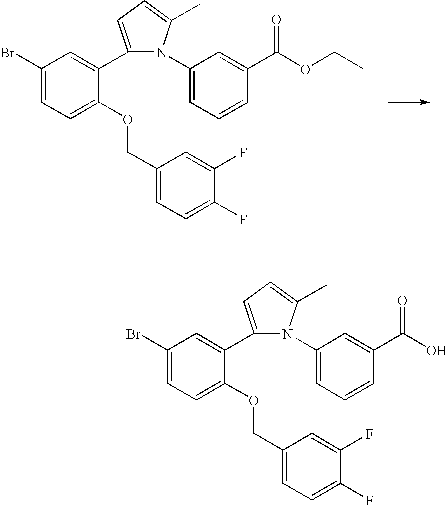

Procedure as for 3-{2-[5-bromo-2-(2,4,6-trifluoro-benzyloxy)-phenyl]-5-methyl-pyrrol-1-yl}-benzoic acid.

¹H-NMR (400 MHz, CDCl₃) 2.16 (3H, s), 4.64 (2H, s), 6.14 (1H, d, J=3 Hz), 6.30 (1H, d, J=3 Hz), 6.48 (1H, d, J=9 Hz), 6.74-6.80 (1H, m), 6.81-6.87 (1H, m), 7.02-7.10 (1H, m), 7.13-7.17 (1H, m), 7.19 (1H, dd, J=3 Hz, 9 Hz), 7.31 (1H, t, J=8 Hz), 7.40 (1H, d, J=2 Hz), 7.76 (1H, t, J=1 Hz), 7.95 (1H, dt, J=1 Hz, 8 Hz).

LC/MS t=4.08 min [MH⁺] 498/500.

### Example 142

### 3-{2-[5-Bromo-2-(4-trifluoromethoxy-benzyloxy)-phenyl]-5-methyl-pyrrol-1-yl}-benzoic acid

### a) 3-{2-[5-Bromo-2-(4-trifluoromethoxy-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.36 min [MH⁺] 574/576.

### b) 3-{2-[5-Bromo-2-(4-trifluoromethoxy-benzyloxy)-phenyl]-5-methyl-pyrrol-1-yl}-benzoic acid

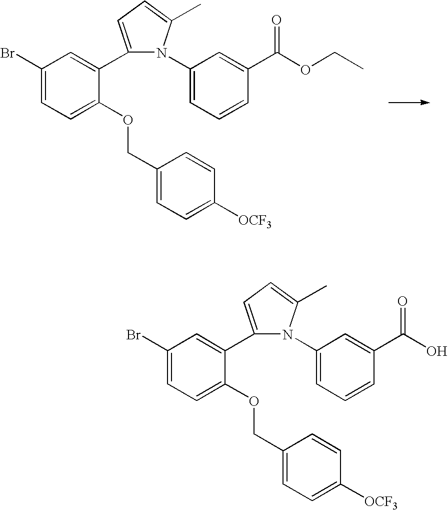

Procedure as for 3-{2-[6-bromo-2-(2,4,6-trifluoro-benzyloxy)-phenyl]-5-methyl-pyrrol-1-yl}-benzoic acid.

LC/MS t=4.20 min [MH⁺] 546/548.

### Example 143

### 3-{2-[5-Bromo-2-(benzo[1,2,5]oxadiazol-5-ylmethoxy)-phenyl]-5-methyl-pyrrol-1-yl}-benzoic acid

### a) 3-{2-[6-Bromo-2-(benzo[1,2,5]oxadiazol-5-ylmethoxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.17 min [MH⁺] 532/534.

### b) 3-{2-[6-Bromo-2-(benzo[1,2,5]oxadiazol-5-ylmethoxy)-phenyl]-5-methyl-pyrrol-1-yl}-benzoic acid

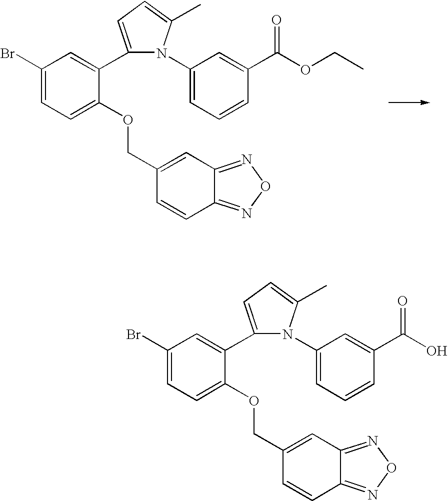

Procedure as for 3-{2-[5-Bromo-2-(2,4,6-trifluoro-benzyloxy)-phenyl]-5-methyl-pyrrol-1-yl}-benzoic acid.

LC/MS t=4.01 min [MH⁺] 504/506.

### Example 144

### 3-{2-[5-Bromo-2-(4-bromo-benzyloxy)-phenyl]-5-methyl-pyrrol-1-yl}-benzoic acid

### a) 3-{2-[5-Bromo-2-(4-bromo-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.37 min [MH⁺] 568/570

### b) 3-{2-[6-Bromo-2-(4-bromo-benzyloxy)-phenyl]-5-methyl-pyrrol-1-yl}-benzoic acid

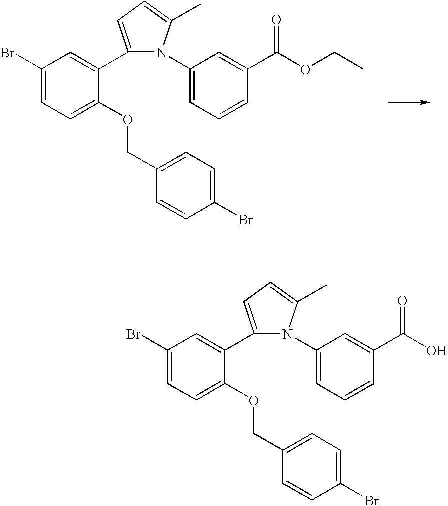

Procedure as for 3-{2-[5-bromo-2-(2,4,6-trifluoro-benzyloxy)-phenyl]-5-methyl-pyrrol-1-yl}-benzoic acid.

¹H-NMR (400 MHz, CDCl₃) 2.09 (3H, s), 4.62 (2H, s), 6.09 (1H, d, J=3 Hz), 6.28 (1H, d, J=3 Hz), 6.43 (1H, d, J=9 Hz), 6.90 (2H, d, J=8 Hz), 7.04-7.12 (2H, m), 7.14-7.21 (1H, m), 7.32 (1H, s), 7.36 (2H, d, J=8 Hz), 7.73 (1H, s), 7.87 (1H, d, J=8 Hz).

LC/MS t=4.23 min [MH⁺] 540/542.

### Example 145

### 3-{2-[5-Bromo-2-(3,5-difluoro-benzyloxy)-phenyl]-5-methyl-pyrrol-1-yl}-benzoic acid

### a) 3-{2-[5-Bromo-2-(3,5-difluoro-benzyloxy)-phenyl]-5-methyl-pyrrol-1-yl}-benzoic acid ethyl ester

Procedure as for 3-{2-[5-bromo-2-(biphenylylmethoxy)-phenyl]-5-methyl-pyrrol-1-yl}-benzoic acid ethyl ester using the appropriate benzyl bromide.

LC/MS t=4.27 min [MH⁺] 526/528.

### b) 3-{2-[5-Bromo-2-(3,5-difluoro-benzyloxy)-phenyl]-5-methyl-pyrrol-1-yl}-benzoic acid

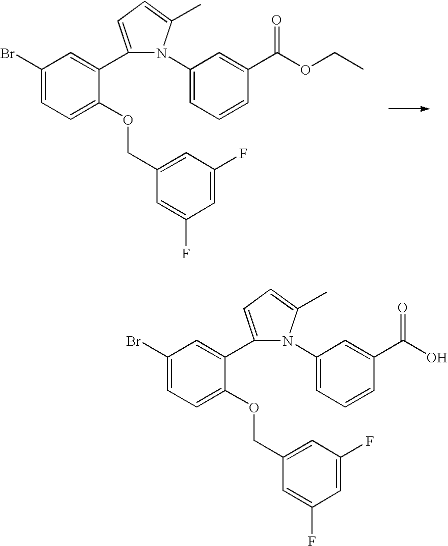

Procedure as for 3-{2-[5-bromo-2-(2,4,6-trifluoro-benzyloxy)-phenyl]-5-methyl-pyrrol-1-yl}-benzoic acid.

LC/MS t=4.09 min [MH⁺] 498/500.

### Example 146

### 3-{2-[5-Bromo-2-(3-methoxy-benzyloxy)-phenyl]-5-methyl-pyrrol-1-yl}-benzoic acid

### a) 3-{2-[5-Bromo-2-(3-methoxy-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.20 min [MH⁺] 520/522.

### b) 3-{2-[5-Bromo-2-(3-methoxy-benzyloxy)-phenyl]-5-methyl-pyrrol-1-yl}-benzoic acid

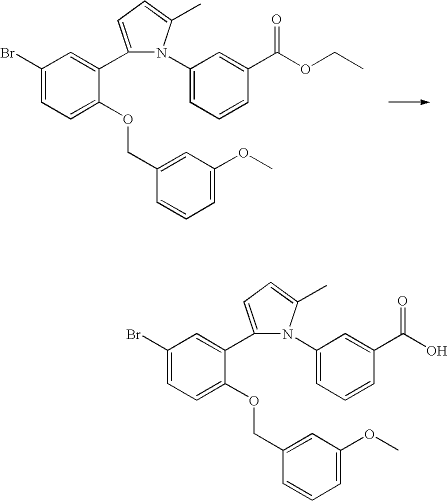

Procedure as for 3-{2-[5-bromo-2-(2,4,6-trifluoro-benzyloxy)-phenyl]-5-methyl-pyrrol-1-yl}-benzoic acid.

LC/MS t=4.02 min [MH⁺] 492/494.

### Example 147

### 3-{2-[5-Bromo-2-(3-fluoro-4-methoxy-benzyloxy)-phenyl]-5-methyl-pyrrol-1-yl}-benzoic acid

### a) 3-{2-[5-Bromo-2-(3-fluoromethoxy-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.18 min [MH⁺] 538/540.

### b) 3-{2-[5-Bromo-2-(3-fluoro-4-methoxy-benzyloxy)-phenyl]-5-methyl-pyrrol-1-yl}-benzoic acid

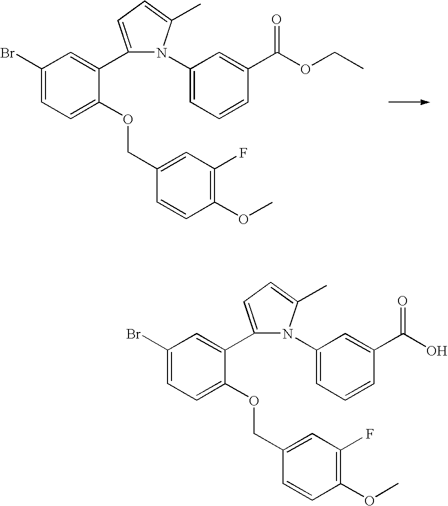

Procedure as for 3-{2-[5-bromo-2-(2,4,6-trifluoro-benzyloxy)-phenyl]-5-methyl-pyrrol-1-yl}-benzoic acid.

LC/MS t=4.01 [MH⁺] 510/512.

### Example 148

### 3-(2-{5-Bromo-2-[3-(1,1-difluoro-methoxy)-benzyloxy]-phenyl}-5-methyl-pyrrol-1-yl)-benzoic acid

### a) 3-(2-{5-Bromo-2-[3-(1,1-difluoro-methoxy)-benzyloxy]-phenyl}-5-methyl-pyrrol-1-yl}-benzoic acid ethyl ester

Procedure as for 3-(2-[5-bromo-2-(biphenyl-4-ylmethoxy)-phenyl]-5-methyl-pyrrol-1-yl}-benzoic acid ethyl ester using the appropriate benzyl bromide.

LC/MS t=4.21 min [MH⁺] 556/568.

### b) 3-(2-{5-Bromo-2-[3-(1,1-difluoro-methoxy)-benzyloxy]-phenyl}-5-methyl-pyrrol-1-yl)-benzoic acid

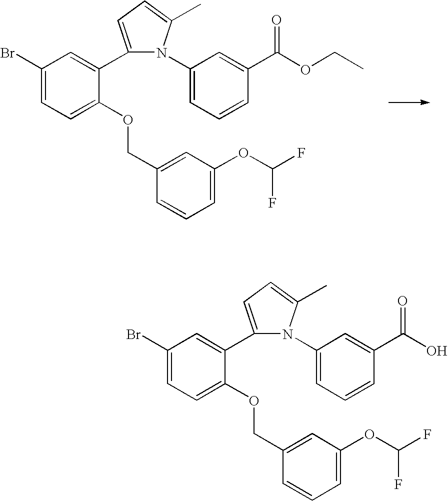

Procedure as for 3-{2-[5bromo-2-(2,4,6trifluoro-benzyloxy)-phenyl]-5-methyl-pyrrol-1-yl}-benzoic acid.

LC/MS t=4.04 min [MH⁺] 528/530.

### Example 149

### 3-{2-[5-Bromo-2-(2,3-difluoro-benzyloxy)-phenyl]-5-methyl-pyrrol-1-yl}-benzoic acid

### a) 3-{2-[5-Bromo-2-(2,3-difluoro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.24 min [MH⁺] 526/528.

### b) 3-{2-[5-Bromo-2-(2,3-difluoro-benzyloxy)-phenyl]-5-methyl-pyrrol-1-yl}-benzoic acid

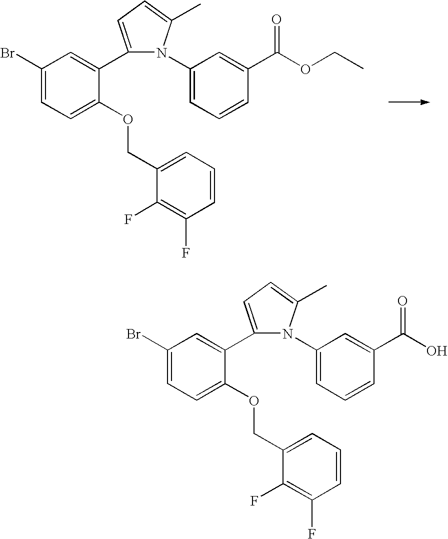

Procedure as for 3-{2-[5-bromo-2-(2,4,6-trifluoro-benzyloxy)-phenyl]-5-methyl-pyrrol-1-yl}-benzoic acid.

¹H-NMR (400 MHz, CDCl₃) 2.15 (3H, s), 4.79 (2H, s), 6.12 (1H, d, J=3 Hz), 6.30 (1H, d, J=3 Hz), 6.53 (1H, d, J=9 Hz), 6.75 (1H, t, J=8 Hz), 6.95-7.01 (1H, m), 7.03-7.09 (1H, m), 7.15-7.18 (1H, m), 7.21 (1H, dd, J=3 Hz, 9 Hz), 7.31 (1H, t, J=8 Hz), 7.37 (1H, d, J=3 Hz), 7.76 (1H, t, J=1 Hz), 7.95 (1H, dt, J=1 Hz, 8 Hz).

LC/MS t=4.07 min [MH⁺] 498/500.

### Example 150

### 3-{2-[5-Bromo-2-(2,6-difluoro-benzyloxy)-phenyl]-5-methyl-pyrrol-1-yl}-benzoic acid

### a) 3-{2-[5-Bromo-2-2,6-difluoro-benzyloxy)-phenyl]-5-methyl-pyrrol-1-yl}-benzoic acid ethyl ester

Procedure as for 3-{2-[5Bromo-2-(biphenyl-4-ylmethoxy)-phenyl]-5-methyl-pyrrol-1-yl}-benzoic acid ethyl ester using the appropriate benzyl bromide.

LC/MS t=4.17 min [MH⁺] 526/528.

### b) 3-{2-[5-Bromo-2-(2,6-difluoro-benzyloxy)-phenyl]-5-methyl-pyrrol-1-yl}-benzoic acid

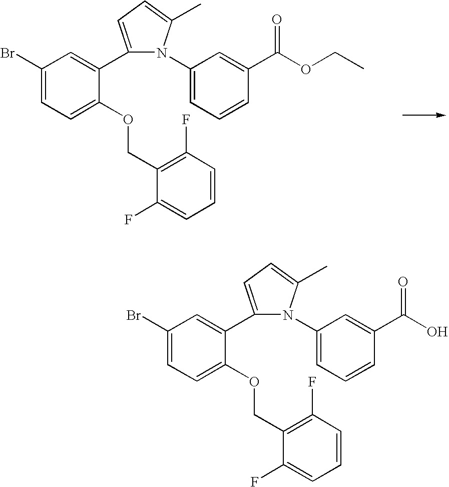

Procedure as for 3-{2-[5-bromo-2-(2,4,6-trifluoro-benzyloxy)-phenyl]-5-methyl-pyrrol-1-yl}-benzoic acid.

¹H-NMR (400 MHz, CDCl₃) 1.95 (3H, s), 4.78 (2H, s), 5.96 (1H, d, J=3 Hz), 6.23 (1H, d, J=3 Hz), 6.64 (1H, d, J=9 Hz), 6.79 (2H, t, J=8 Hz), 6.94-7.19 (5H, m), 7.73 (1H, t, J=1 Hz), 7.75-7.80 (1H, m).

LC/MS [MH⁺] 498/500.

### Example 151

### 3-{2-[5-Bromo-2-(naphthalen-2-ylmethoxy)-phenyl]-5-methyl-pyrrol-1-yl}-benzoic acid

### a) 3-{2-[5-Bromo-2-(naphthalen-2-ylmethoxy)-phenyl]-5-methylpyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.40 [MH⁺] 540/542.

### b) 3-{2-[5-Bromo-2-(naphthalen-2-ylmethoxy)-phenyl]-5-methyl-pyrrol-1-yl}-benzoic acid

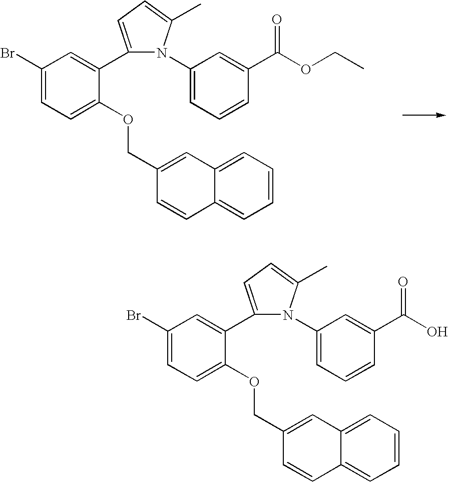

Procedure as for 3-{2-[5-bromo-2-(2,4,6-trifluoro-benzyloxy)-phenyl]-5-methyl-pyrrol-1-yl}-benzoic acid.

LC/MS t=4.28 min [MH⁺] 512/514.

### Example 152

### 3-{2-[5-Bromo-2-(4-methyl-benzyloxy)-phenyl]-5-methyl-pyrrol-1-yl}-benzoic acid

### a) 3-{2-[6-Bromo-2-(4-methyl-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.31 min [MH⁺] 504/506.

### b) 3-{2-[5-Bromo-2-(4-methyl-benzyloxy)-phenyl]-5-methyl-pyrrol-1-yl}-benzoic acid

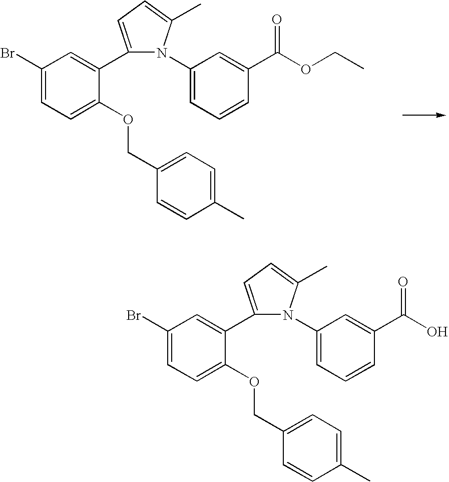

Procedure as for 3-{2-[5-bromo-2-(2,4,6-trifluoro-benzyloxy)-phenyl]-5-methyl-pyrrol-1-yl}-benzoic acid using the appropriate benzyl bromide.

¹H-NMR (400 MHz, CDCl₃) 2.14 (3H, s), 2.30 (3H, s), 4.69 (2H, s), 6.12 (1H, d, J=3 Hz), 6.31 (1H, d, J=3 Hz), 6.50 (1H, d, J=9 Hz), 6.92 (2H, d, J=8 Hz), 7.07 (2H, d, J=8 Hz), 7.12-7.16 (2H, m), 7.28 (1H, t, J=8 Hz), 7.34 (1H, d, J=3 Hz), 7.78 (1H, t, J=1 Hz), 7.93 (1H, dt, J=1 Hz, 8 Hz).

LC/MS=t=4.14 min [MH⁺] 476/478.

### Example 153

### 3-{2-[5-Bromo-2-(3,5-dichloro-benzyloxy)-phenyl]-5-methyl-pyrrol-1-yl)--benzoic acid

### a) 3-{2-[5-Bromo-2-(3,5-dichloro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.49 min [MH⁺] 558/560.

### b) 3-{2-[5-Bromo-2-3,5-dichloro-benzyloxy)-phenyl]-5-methyl-pyrrol-1-yl}-benzoic acid

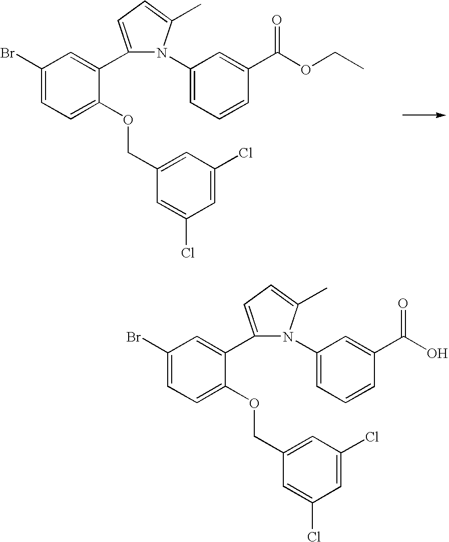

Procedure as for 3-{2-[5-bromo-2-(2,4,6-trifluoro-benzyloxy)-phenyl]-5-methyl-pyrrol-1-yl}-benzoic acid.

LC/MS t=4.39 min [MH⁺] 530/532.

### Example 154

### 3-{2-[5-Bromo-2-(2,3,5-trifluoro-benzyloxy)-phenyl]-5-methyl-pyrrol-1-yl}-benzoic acid

### a) 3-{2-[5-Bromo-2-(2,3,5-trifluoro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.28 min, [MH⁺] 544/546.

### b) 3-{2-[5-Bromo-2-(2,3,5-trifluoro-benzyloxy)-phenyl]-5-methyl-pyrrol-1-yl}-benzoic acid

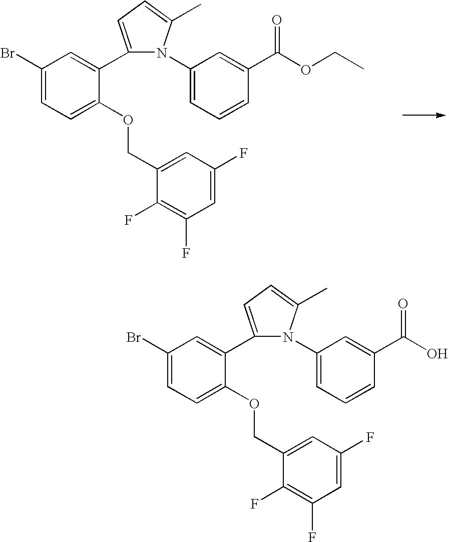

Procedure as for 3-{2-[5-bromo-2-(2,4,6-trifluoro-benzyloxy)-phenyl]-5-methyl-pyrrol-1-yl}-benzoic acid.

LC/MS t=4.10 min [MH⁺] 516/518.

### Example 155

### 3-{2-[5-Bromo-2-(2,4,6-trifluoro-benzyloxy)-phenyl]-5-methyl-pyrrol-1-yl}-benzoic acid

### a) 3-{2-[5-Bromo-2-(2,4,6-trifluoro-benzyloxy)-phenyl]-5-methyl-pyrrol-1-yl}-benzoic acid ethyl ester

Procedure as for 3-{2-[5-bromo-2-(biphenyl-4-ylmethoxy)-phenyl]-5-methyl-pyrrol-1-yl}-benzoic acid ethyl ester using the appropriate benzyl bromide.

LC/MS t=4.21 min [MH⁺] 544/546.

### b) 3-{2-[5-Bromo-2-(2,4,6-trifluoro-benzyloxy)-phenyl]-5-methyl-pyrrol-1-yl}-benzoic acid

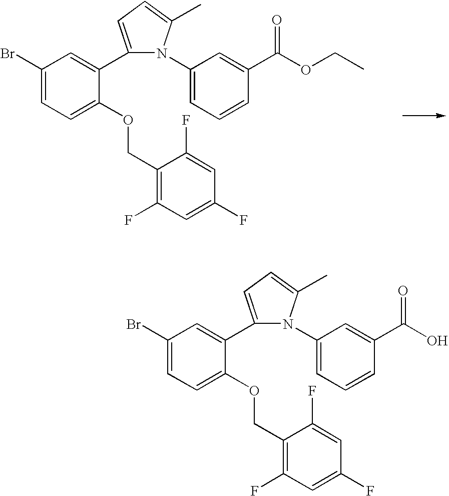

3-{2-[5-Bromo-2-(2,4,6-trifluoro-benzyloxy)-phenyl]-5-methyl-pyrrol-1-yl}-benzoic acid ethyl ester (0.06g) was dissolved in EtOH (1 ml) and stirred with 2M NaOH (0.5 ml) at reflux in a sealed vessel for 30 minutes. The solvent was removed in vacuo, the residue taken up in DCM and washed with dil. citric acid. The organics were separated using a phase separator column with a NaSO₄ cartridge attached. The solvent was removed in vacuo and the residue freeze-dried in a MeCN/H₂O solution to yield a yellow solid (0.054g, 95%).

¹H-NMR (400 MHz, CDCl₃) 2.14 (3H, s), 4.74 (2H, s), 6.08 (1H, d, J=3 Hz), 6.24 (1H, d, J=3 Hz), 6.66 (2H, t, J=8 Hz), 6.72 (1H, d, J=9 Hz), 7.14 (1H, m), 7.22-7.28 (2H, m), 7.34 (1H, t, J=8 Hz), 7.78 (1H, t, J=1 Hz), 7.99 (1H, dt, J=1 Hz, J=8 Hz).

LC/MS t=3.99 min [MH⁺] 516/518.

### Example 156

### 3-{2-[5-Bromo-2-(2-methyl-benzyloxy)-phenyl]-5-methyl-pyrrol-1-yl}-benzoic acid

### a) 3-{2-[5-Bromo-2-(2-methyl-benzyloxy)-phenyl]-5-methyl-pyrrol-1-yl}-benzoic acid ethyl ester

3-[2-(5-Bromo-2-hydroxy-phenyl]-5-methyl-pyrrol-1-yl}-benzoic acid ethyl ester (0.15g, 0.375 mmol), potassium carbonate (0.104 g, 0.75 mmol) and 2-methylbenzyl bromide (0.06 ml, 0.413 mmol) were stirred in DMF at 50° C. for 2.5 hours under a nitrogen atmosphere. The reaction was diluted with EtOAc and washed with 2×H₂O and brine, dried over MgSO₄, filtered and concentrated in vacuo. The resultant oil was then purified using column chromatography on a SPE cartridge (20g) eluting in 20% EtOAc/i-hex.

LC/MS t=4.34 min [MH⁺] 504/506.

### b) 3-{2-[5-Bromo-2-(2-methyl-benzyloxy)-phenyl]-5-methyl-pyrrol-1-yl}-benzoic acid

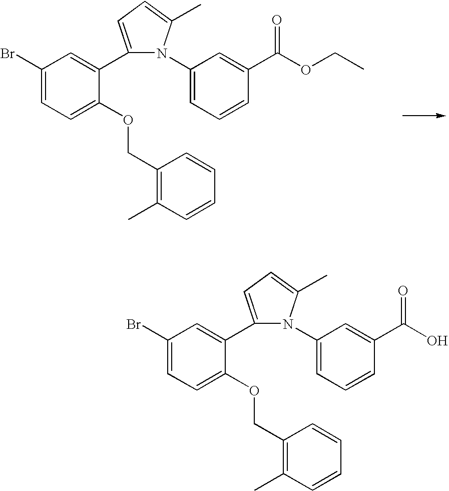

Procedure as for 3-[2-(5-bromo-2-benzyloxy-phenyl)-5-methyl-pyrrol-1-yl]-5-trifluoromethyl-benzoic acid.

LC/MS t=4.10 min [MH⁺] 476/478.

### Example 157

### 3-{2-[5-Bromo-2-(2-fluoro-benzyloxy)-phenyl]-5-methyl-pyrrol-1-yl}-benzoic acid

### a) 3-{2-[5-Bromo-2-(2-fluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.27 min [MH⁺] 508/510.

### b) 3-{2-[5-Bromo-2-(2-fluoro-benzyloxy)-phenyl]-5-methyl-pyrrol-1-yl}-benzoic acid

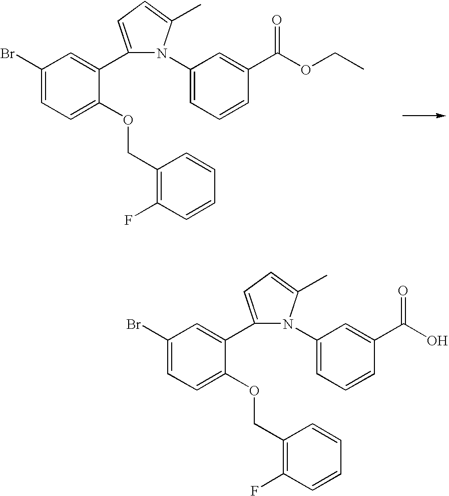

Procedure as for 3-[2-(5-bromo-2-benzyloxy-phenyl)-5-methyl-pyrrol-1-yl]-5-trifluoromethyl-benzoic acid.

¹H-NMR (400 MHz, CDCl₃) 2.14 (3H, s), 4.79 (2H, s), 6.12 (1H, d, J=3 Hz), 6.31 (1H, d, J=3 Hz), 6.53 (1H, d, J=9 Hz), 6.94-7.06 (3H, m), 7.12-7.24 (3H, m), 7.25-7.32 (1H+ CDCl₃, m), 7.34 (1H, d, J=2 Hz), 7.79 (1H, s), 7.94 (1H, d, J=7 Hz).

LC/MS t=4.01 min [MH⁺] 480/482.

### Example 158

### 3-{2-[5-Bromo-2-(2,3,6-trifluoro-benzyloxy)-phenyl]-5-methyl-pyrrol-1-yl}-benzoic acid

### a) 3-{2-[5-Bromo-2-(2,3,6-trifluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hexane.

LC/MS t=4.23 min [MH⁺] 544/546.

### b) 3-{2-[5-Bromo-2-(2,3,6-trifluoro-benzyloxy)-phenyl]-5-methyl-pyrrol-1-yl}-benzoic acid

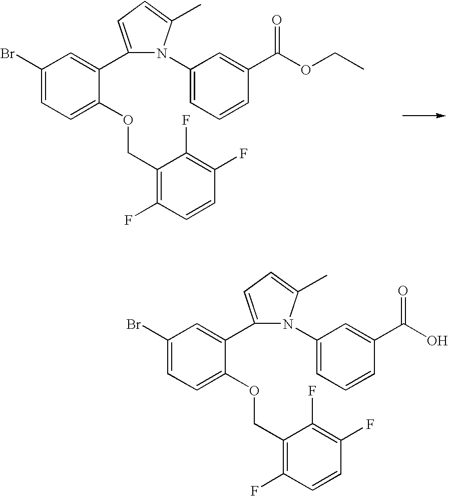

Procedure as for 3-[2-(5-bromo-2-benzyloxy-phenyl)-5-methyl-pyrrol-1-yl]-5-trifluoromethyl-benzoic acid.

¹H-NMR (400 MHz, MeOD) 2.08 (3H, s), 4.83 (2H, s), 6.00 (1H, d, J=3 Hz), 6.13 (1H, d, J=3 Hz), 6.87 (1H, d, J=9 Hz), 6.92-7.00 (1H, m), 7.09 (1H, d, J=8 Hz), 7.22-7.37 (4H, m), 7.60 (1H, s), 7.89 (1H, d, J=8 Hz).

LC/MS t=3.97 min [MH⁺] 516/518.

### Example 159

### 3-{2-[5-Bromo-2-(2-chloro-benzyloxy)-phenyl]-5-methyl-pyrrol-1-yl}-benzoic acid

### a) 3-{2-[5-Bromo-2-(2-chloro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20 g) eluting in 20% EtOAc/i-hex.

LC/MS t=4.18 min [MH⁺] 524/526.

### b) 3-{2-[5-Bromo-2-(2-chloro-benzyloxy)-phenyl]-5-methyl-pyrrol-1-yl}-benzoic acid

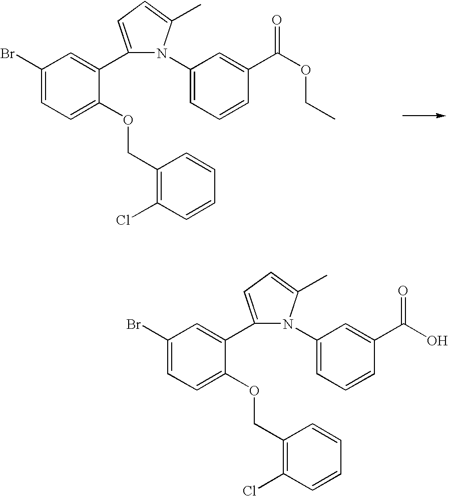

Procedure as for 3-[2-(5-bromo-2-benzyloxy-phenyl]-5-methyl-pyrrol-1-yl}-5-trifluoromethyl-benzoic acid.

LC/MS t=4.17 min [MH⁺] 496/498/500.

### Example 160

### 3-{2-[5-Bromo-2-(2,6-dichloro-benzyloxy)-phenyl]-5-methyl-pyrrol-1-yl}-benzoic acid

### a) 3-{2-[5-Bromo-2-(2,6-dichloro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 10% EtOAc/i-hex.

LC/MS t=4.18 min [MH⁺] 558/560/562/564.

### b) 3-{2-[5-Bromo-2-(2,6-dichloro-benzyloxy)-phenyl]-5-methyl-pyrrol-1-yl}-benzoic acid

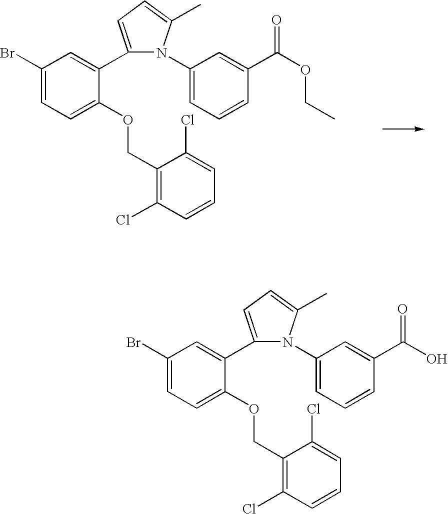

Procedure as for 3-[2-(5-bromo-2-benzyloxy-phenyl]-5-methyl-pyrrol-1-yl}-5-trifluoromethyl-benzoic acid.

LC/MS t=4.16 min [MH⁺] 530/532/534/536.

### Example 161

### 3-{2-[5-Bromo-2-(2,4-bis-trifluoromethyl-benzyloxy)-phenyl]-5-methyl-pyrrol-1-yl}-benzoic acid

### a) 3-{2-[6-Bromo-2-(2,4-bis-trifluoromethyl-benzyloxy)-phenyl]-1-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 10% EtOAc/i-hex.

LC/MS t=4.53 min [MH⁺] 626/628.

### b) 3-{2-[5-Bromo-2-(2,4-bis-trifluoromethyl-benzyloxy)-phenyl]-5-methyl-pyrrol-1-yl}-benzoic acid

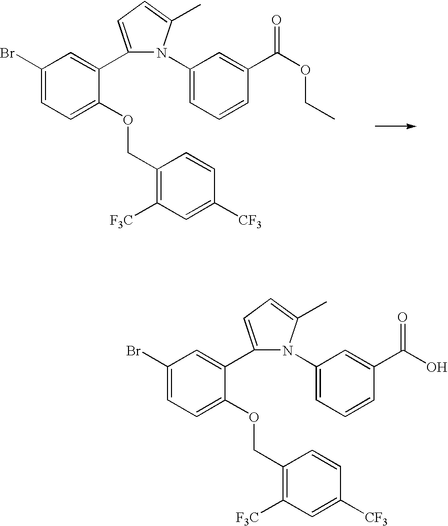

Procedure as for 3-[2-(5-bromo-2-benzyloxy-phenyl)-5-methyl-pyrrol-1-yl]-5-trifluoromethyl-benzoic acid.

LC/MS t=4.29 min [MH⁺] 598/600.

### Example 162

### 3-{2-[5-Bromo-2-2,5-difluoro-benzyloxy)-phenyl]-5-methyl-pyrrol-1-yl}-benzoic acid

### a) 3-{2-[5-Bromo-2-(2,5-difluoro-benzyloxy)-phenyl]-5-methyl-pyrrol-1-yl}-benzoic acid ethyl ester

Procedure as for 3-{2-[5bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 10% EtOAc/i-hex.

LC/MS t=4.28 min [MH⁺] 526/528.

### b) 3-{2-[5-Bromo-2-(2,5-difluoro-benzyloxy)-phenyl]-5-methyl-pyrrol-1-yl}-benzoic acid

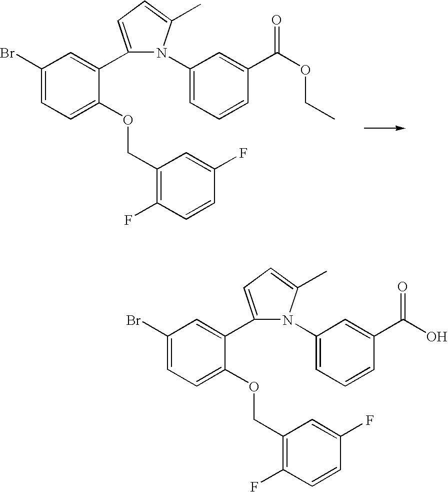

Procedure as for 3-[2-(5bromo-2-benzyloxy-phenyl)-5-methyl-pyrrol-1-yl]-5-trifluoromethyl-benzoic acid.

¹H-NMR (400 MHz, CDCl₃) 2.17 (3H, s), 4.77 (2H, s), 6.15 (1H, d, J=3 Hz), 6.32 (1H, d, J=3 Hz), 6.52 (1H, d, J=9 Hz), 6.64-6.70 (1H, m), 6.86-6.99 (2H, m), 7.17-7.24 (2H, m), 7.33 (1H, t, J=8 Hz), 7.39 (1H, d, J=2 Hz), 7.81 (1H, s), 7.97 (1H, d, J=8 Hz).

LC/MS t=4.03 min [MH⁺] 498/500.

### Example 163

### 3-{2-[5-Bromo-2-(4-trifluoromethyl-benzyloxy)-phenyl]-5-methyl-pyrrol-1-yl}-benzoic acid

### a) 3-{2-[5-Bromo-2-(4-trifluoromethyl-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 10% EtOAc/i-hex.

LC/MS t=4.38 min [MH⁺] 558/560.

### b) 3-{2-[5-Bromo-2-(4-trifluoromethyl-benzyloxy)-phenyl]-5-methyl-pyrrol-1-yl}-benzoic acid

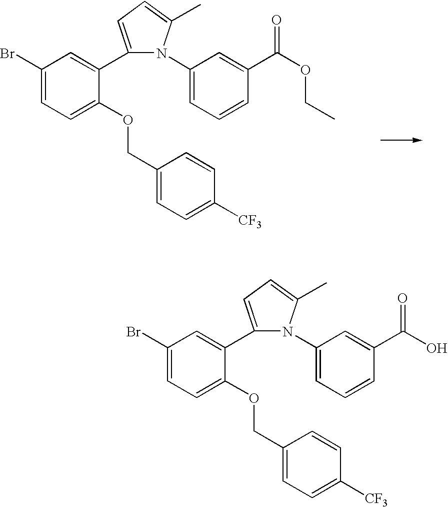

Procedure as for 3-[2-(5-bromo-2-benzyloxy-phenyl)-5-methyl-pyrrol-1-yl]-5-trifluoromethyl-benzoic acid, however further purification was required on the MDAP.

LC/MS t=4.15 min [MH⁺] 530/532.

### Example 164

### 3-{2-[5-Bromo-2-(2-chloro-6-fluoro-benzyloxy)-phenyl]-5-methyl-pyrrol-1-yl}-benzoic acid

### a) 3-{2-[5-Bromo-2-(2-chloro-6-fluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.30 min [MH⁺] 542/544/546.

### b) 3-{2-[5-Bromo-2-(2-chloro-6-fluoro-benzyloxy)-phenyl]-5-methyl-pyrrol-1-yl}-benzoic acid

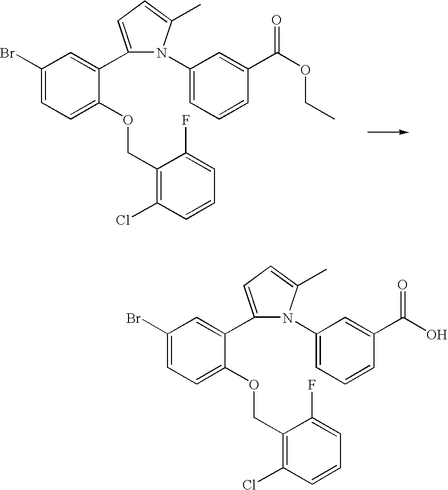

Procedure as for 3-[2-(5-bromo-2-benzyloxy-phenyl)-5-methyl-pyrrol-1-yl]-5-trifluoromethyl-benzoic acid.

LC/MS t=4.06 min [MH⁺] 514/516/518.

### Example 165

### 3-{2-[5-Bromo-2-(3,4,5-trifluoro-benzyloxy)-phenyl]-5-methyl-pyrrol-1-yl}-benzoic acid

### a) 3-{2-[5-Bromo-2-(3,4,5-trifluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.35 min [MH⁺] 544/546.

### b) 3-{2-[5-Bromo-2-(3,4,5-trifluoro-benzyloxy)-phenyl]-5-methyl-pyrrol-1-yl}-benzoic acid

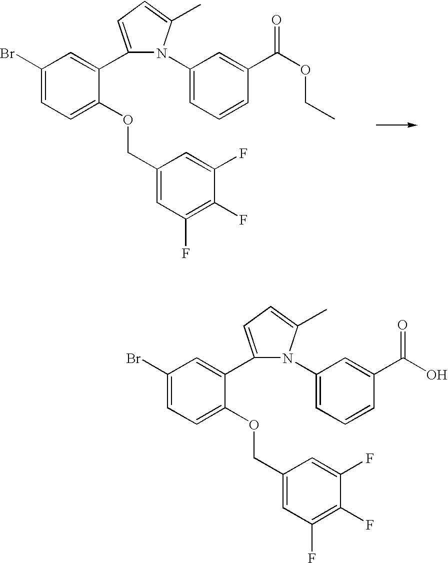

Procedure as for 3-[2-(5-bromo-2-benzyloxy-phenyl)-5-methyl-pyrrol-1-yl]-5-trifluoromethyl-benzoic acid.

LC/MS t=4.12 min [MH⁺] 516/518.

### Example 166

### 3-{2-[5-Bromo-2-(2-bromo-5-fluoro-benzyloxy)-phenyl]-5-methyl-pyrrol-1-yl}-benzoic acid

### a) 3-{2-[5-Bromo-2-(2-bromo-5-fluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.45 min [MH⁺] 586/588/590.

### b) 3-{2-[5-Bromo-2-(2-bromo-5-fluoro-benzyloxy)-phenyl]-5-methyl-pyrrol-1-yl}-benzoic acid

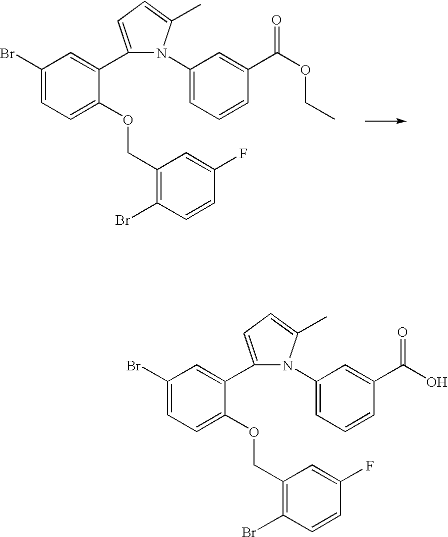

Procedure as for 3-[2-(5-bromo-2-benzyloxy-phenyl)-5-methyl-pyrrol-1-yl]-5-trifluoromethyl-benzoic acid.

LC/MS t=4.25 min [MH⁺] 558/560/562.

### Example 167

### 3-{2-[5-Bromo-2-(2,4-dichloro-5-fluoro-benzyloxy)-phenyl]-5-methyl-pyrrol-1-yl}-benzoic acid

### a) 3-{2-[5-Bromo-2-(2,4-dichloro-5-fluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.34 min

### b) 3-{2-[5-Bromo-2-(2,4-dichloro-5-fluoro-benzyloxy)-phenyl]-5-methyl-pyrrol-1-yl}-benzoic acid

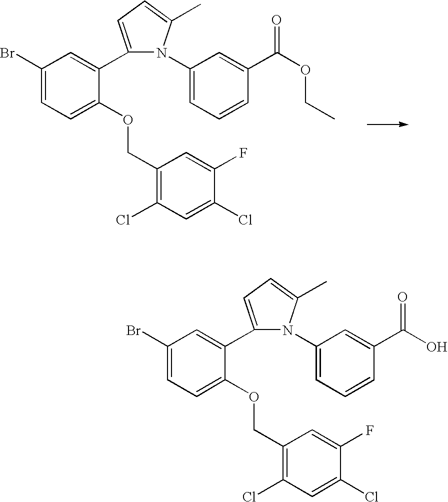

Procedure as for 3-[2-(5-bromo-2-benzyloxy-phenyl)-5-methyl-pyrrol-1-yl]-5-trifluoromethyl-benzoic acid.

LC/MS t=4.16 min 548/550/552.

### Example 168

### 3-{2-[5-Bromo-2-(2,4,5-trifluoro-benzyloxy)-phenyl]-5-methyl-pyrrol-1-yl}-benzoic acid

### a) 3-{2-[5-Bromo-2-(2,4,5-trifluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.11 min [MH⁺] 544/546.

### b) 3-{2-[5-Bromo-2-(2,4,5-trifluoro-benzyloxy)-phenyl]-5-methyl-pyrrol-1-yl}-benzoic acid

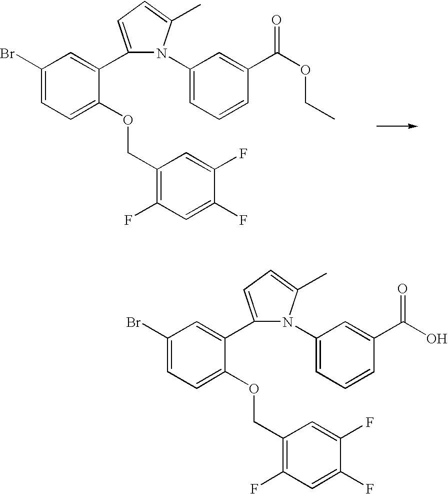

Procedure as for 3-[2-(5-bromo-2-benzyloxy-phenyl)-5-methyl-pyrrol-1-yl]-5-trifluoromethyl-benzoic acid.

¹H-NMR (400 MHz, CDCl₃) 1.98 (3H, s), 4.68 (2H, s), 5.45-5.80 (1H, broad s), 6.03 (1H, d, J=3 Hz), 6.26 (1H, d, J=3 Hz), 6.42 (1H, d, J=9 Hz), 6.70-6.86 (2H, m), 6.93-7.05 (3H, m), 7.21 (1H, d, J=2 Hz), 7.72-7.75 (2H, m).

LC/MS t=3.90 min [MH⁺] 516/518.

### Example 169

### 3-{2-[5-Bromo-2-(2-fluoro-4-trifluoromethyl-benzyloxy)-phenyl]-5-methyl-pyrrol-1-yl}-benzoic acid

### a) 3-{2-[5-Bromo-2-(2-fluoro-4-trifluoromethyl-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.19 min [MH⁺] 576/578.

### b) 3-{2-[6-Bromo-2-(2-fluoro-4-trifluoromethyl-benzyloxy)-phenyl]-5-methyl-pyrrol-1-yl}-benzoic acid

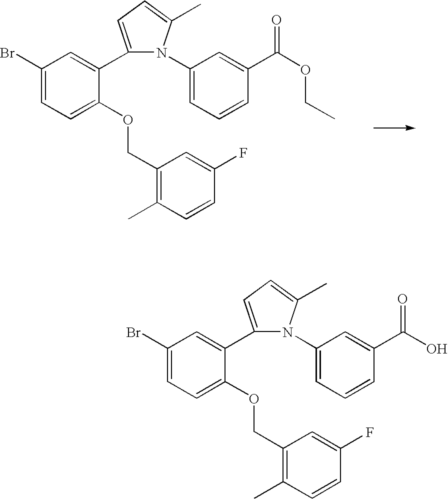

Procedure as for 3-[2-(5-bromo-2-benzyloxy-phenyl)-5-methyl-pyrrol-1-yl]-5-trifluoromethyl-benzoic acid.

LC/MS t=3.96 min [MH⁺] 548/550.

### Example 170

### 3-{2-[5-Bromo-2-(5-fluoro-2-methyl-benzyloxy)-phenyl]-5-methyl-pyrrol-1-yl}-benzoic acid

### a) 3-{2-[5-Bromo-2-(5-fluoro-2-methyl-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.34 min [MH⁺] 522/524.

### b) 3-{2-[5-Bromo-2-(5-fluoro-2-methyl-benzyloxy)-phenyl]-5-methyl-pyrrol-1-yl}-benzoic acid

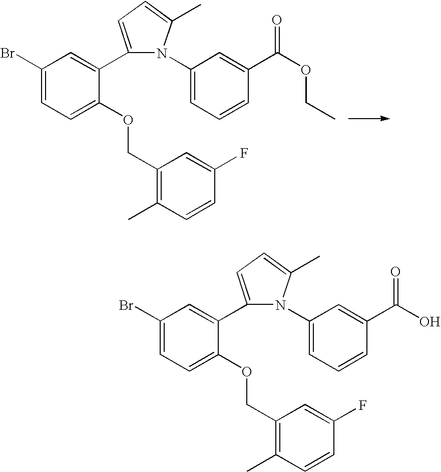

Procedure as for 3-[2-(5-bromo-2-benzyloxy-phenyl)-5-methyl-pyrrol-1-yl]-5-trifluoromethyl-benzoic acid.

LC/MS t=4.11 min [MH⁺] 494/496.

### Example 171

### 3-{2-[5-Bromo-2-(2,3,4-trifluoro-benzyloxy)-phenyl]-5-methyl-pyrrol-1-yl}-benzoic acid

### a) 3-{2-[5-Bromo-2-(2,3,4-trifluoro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (209) eluting in 20% EtOAc/i-hex.

LC/MS t=4.31 min [MH⁺] 544/546.

### b) 3-{2-[5-Bromo-2-(2,3,4-trifluoro-benzyloxy)-phenyl]-5-methyl-pyrrol-1-yl}-benzoic acid

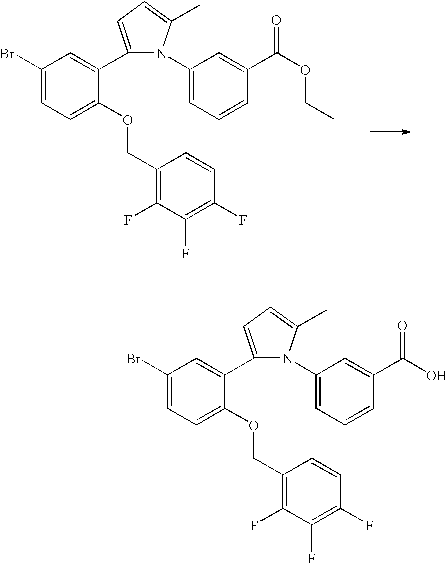

Procedure as for 3-[2-(5-bromo-2-benzyloxy-phenyl}-5-methyl-pyrrol-1-yl}-5-trifluoromethyl-benzoic acid.

LC/MS t=4.07 min [MH⁺] 514/516.

### Example 172

### 3-{2-[5-Bromo-2-(2-fluoro-6-trifluoromethyl-benzyloxy)-phenyl]-5-methyl-pyrrol-1-yl}-benzoic acid

### a) 3-{2-[5-Bromo-2-(2-fluoro-6-trifluoromethyl-benzyloxy)-phenyl]-3-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.29 min [MH⁺] 576/578.

### b) 3-{2-[5-Bromo-2-(2-fluoro-6-trifluoromethyl-benzyloxy)-phenyl]-5-methyl-pyrrol-1-yl}-benzoic acid

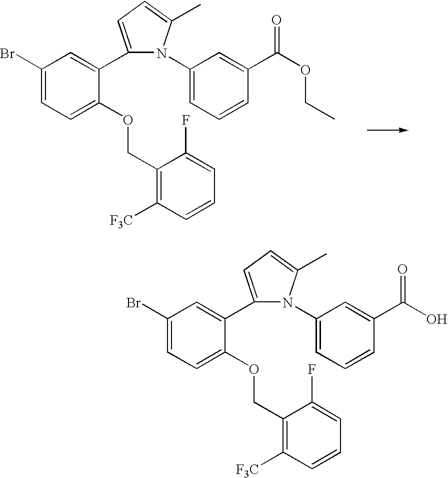

Procedure as for 3-[2-(5-bromo-2-benzyloxy-phenyl)-5-methyl-pyrrol-1-yl-5-trifluoromethyl-benzoic acid.

LC/MS t=4.04 min [MH⁺] 548/550.

### Example 173

### a) 3-{2-[5-Bromo-2-(2-bromo-benzyloxy)-phenyl]-5-methyl-pyrrol-1-yl}-benzoic acid

### a) 3-{2-[5-Bromo-2-(2-bromo-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.29 min

### b) 3-{2-[5-Bromo-2-(2-bromo-benzyloxy)-phenyl]-5-methyl-pyrrol-1-yl}-benzoic acid

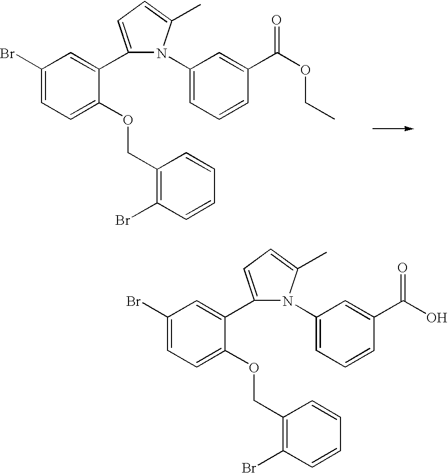

Procedure as for 3-[2-(5-bromo-2-benzyloxy-phenyl)-5-methyl-pyrrol-1-yl]-5-trifluoromethyl-benzoic acid, however further purification was required using column chromatography on the Biotage® Horizon system on a 25S reverse phase column eluting in a gradient of 30-100% MeCN/H₂O.

LC/MS t=4.01 min [MH⁺] 540/542/544.

### Example 174

### 3-{2-[5-Bromo-2-(3-chloro-benzyloxy)-phenyl]-5-methyl-pyrrol-1-yl]-benzoic acid

### a) 3-{2-[5-Bromo-2-(3-chloro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (20g) eluting in 20% EtOAc/i-hex.

LC/MS t=4.39 min

### b) 3-{2-[5-Bromo-2-(3-chloro-benzyloxy)-phenyl]-5-methyl-pyrrol-1-yl}-benzoic acid

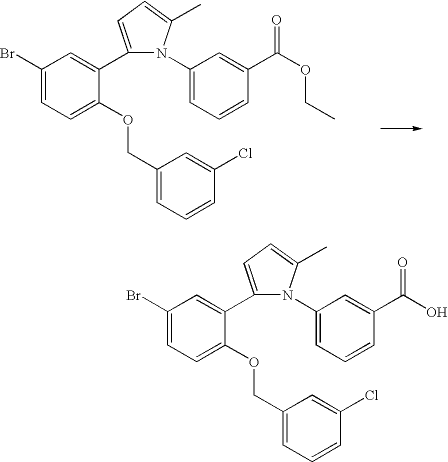

Procedure as for 3-[2-(5-bromo-2-benzyloxy-phenyl)-5-methyl-pyrrol-1-yl]-5-trifluoromethyl-benzoic acid.

LC/MS t=4.18 min [MH⁺] 496/498.

### Example 175

### 3-(2-[5-Bromo-2-(2,4-dichloro-benzyloxy)-phenyl]-5-methyl-pyrrol-1-yl}-benzoic acid

### a) 3-{2-[5-Bromo-2-(2,4-dichloro-benzyloxy)-phenyl]-5-methyl-pyrrol-1-yl}-benzoic acid ethyl ester

Procedure as for 3-{2-[5-bromo-2-(2-methyl-benzyloxy)-phenyl]-5-methyl-pyrrol-1-yl}-benzoic acid ethyl ester using the appropriate benzyl bromide, however purification achieved using column chromatography on a SPE cartridge (25g) eluting in 20% EtOAc/i-hex.

LC/MS t=4.54 min [MH⁺] 558/560/562.

### b) 3-{2-[5-Bromo-2-(2,4-dichloro-benzyloxy)-phenyl]-5-methyl-pyrrol-1-yl}-benzoic acid

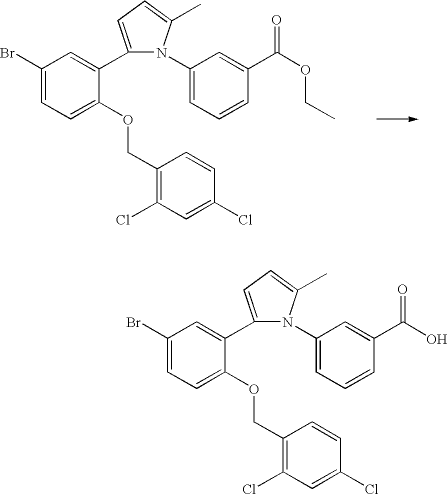

Procedure as for 3-[2-(5-bromo-2-benzyloxy-phenyl)-5-methyl-pyrrol-1-yl]-5trifluoromethyl-benzoic acid, however further purification was required using the MDAP.

¹H-NMR (400 MHz, CDCl₃) 2.18 (3H, s), 4.74 (2H, s), 6.15 (1H, d, J=3 Hz), 6.32 (1H, d, J=3 Hz), 6.48 (1H, d, J=9 Hz), 6.90 (1H, d, J=8 Hz), 7.15 (1H, dd, J=2 Hz, 8 Hz), 7.18-7.23 (2H, m), 7.32 (1H, d, J=2 Hz), 7.35 (1H, t, J=8 Hz), 7.40 (1H, d, J=2 Hz), 7.78-7.81 (1H, m), 7.99 (1H, d, J=8 Hz).

LC/MS t=4.37 min [MH⁺] 530/532/534.

### Example 176

### 3-{2-[5-Bromo-2-(benzyloxy)-phenyl]-5-methyl-pyrrol-1-yl}-acetylamino-benzoic acid

### a) 3-{2-[5-Bromo-2-(benzyloxy)-phenyl]-5-methyl-pyrrol-1-yl}-6-acetylamino-benzoic acid methyl ester

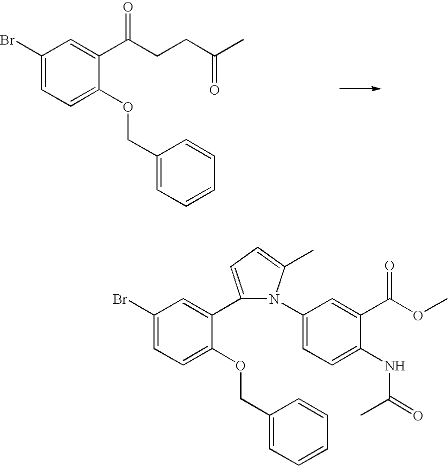

1-(2-Benzyloxy-5-bromo-phenyl)-pentane-1,4-dione (0.10g, 0.277 mmol), 6-acetylamino-3-amino-benzoic acid methyl ester (0.063g, 0.305 mmol) and p-TSA (cat.) in NMP (2 ml) were heated in a sealed vessel at 150° C. for 10 minutes using microwaves. Upon cooling the reaction was diluted with Et₂O and washed with dil. citric acid. The organic layer was extracted and the aqueous layer washed with 3xEt₂O, the combined organic extracts were then washed with brine, dried over MgSO₄, filtered and concentrated in vacuo. The residue was purified by chromatography on silica gel eluting in 20% EtOAc/i-hex. This yielded a white solid (0.1 12g, 60%).

LC/MS t=4.11 min [MH⁺] 533/535.

### b) 3-{2-[5-Bromo-2-(benzyloxy)-phenyl]-5-methyl-pyrrol-1-yl}-6-acetylamino-benzoic acid

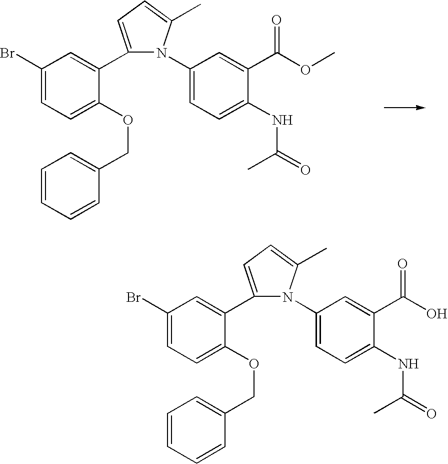

3-{2-[5-Bromo-2-(benzyloxy)-phenyl]-5-methyl-pyrrol-1-yl}-6-acetylamino-benzoic acid ethyl ester (0.112g) was dissolved in MeOH (2 ml) and heated with 2N NaOH (1 ml) in a sealed vessel at 100° C. for 30 minutes. Upon cooling the reaction was diluted with EtOAc and washed with dil. citric acid and brine, dried over MgSO₄, filtered and concentrated in vacuo to yield the title compound as a yellow solid (0.075g, 69%).

¹H-NMR (400 MHz, CDCl₃) 2.13 (3H, s), 2.23 (3H, s), 4.77 (2H, s), 6.12 (1H, d, J=3 Hz), 6.28 (1H, d, J=3 Hz), 6.53 (1H, d, J=9 Hz), 7.05-7.09 (2H, m), 7.16 (1H, dd, J=2 Hz, 9 Hz), 7.20 (1H, dd, J=2 Hz, 9 Hz). 7.25-7.29 (3H+ CDCl₃, m), 7.40 (1H, d, J=2 Hz), 7.74 (1H, d, J=2 Hz), 8.60 (1H, d, J=9 Hz), 10.90 (1H, s).

LC/MS t=4.30 min [MH⁺] 519/521.

### Example 177

### 3-{2-[5-Bromo-2-(benzyloxy)-phenyl]-5-methyl-pyrrol-1-yl}-6-(1,1-difluoro-methoxy)-benzoic acid

### a) 2-Difluoromethoxy-5-nitro-benzoic acid methyl ester

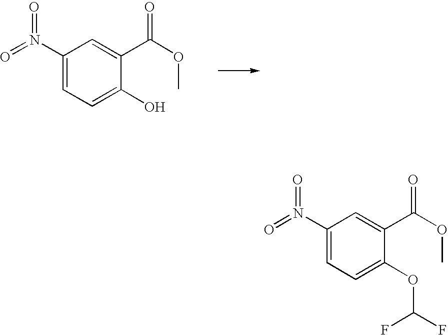

Methyl-5-nitrosalicylate (ex Pfaltz and Bauer) (4.792g), sodium chlorodifluoroacetate (4.444g) and sodium carbonate (3.147g) were heated at 100° C. in DMF (97 mL) for 2.5 hours. Upon cooling, the mixture was partitioned between Et₂O and water. The layers were separated and the aqueous phase was extracted further with Et₂O. The combined extracts were dried (Na₂SO₄), filtered and evaporated to give the title compound, which was used without further purification.

¹H NMR (400 MHz, CDCl₃) 3.98 (3H, s), 6.72 (1H, t, J=73 Hz), 7.45 (1H, d, J=9 Hz), 8.42 (1H, dd, J=3 Hz, J=9 Hz), 8.76 (1H, d, J=3 Hz).

### b) 5-Amino-2-difluoromethoxy-benzoic acid methyl ester

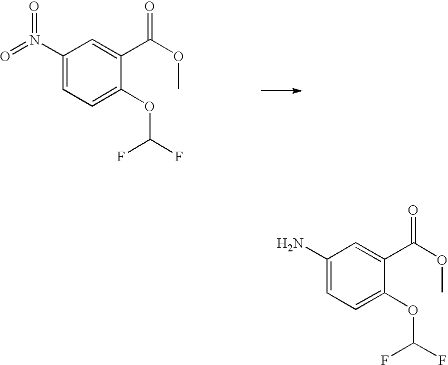

2-Difluoromethoxy-5-nitro-benzoic acid methyl ester (2.5g, 10 mmol) was hydrogenated at atmospheric temperature and pressure in methanol (50 ml) with palladium on charcoal 5% wet (0.4 g) for six hours. The reaction mixture was then filtered through high-flo and evaporated down to an oil which turned into a solid to give the tile compound (2.15g, 99%).

LC/MS t=2.51 min [MH⁺] 218, [MH⁻] 216

### c) 3-{2-[5-Bromo-2-(benzyloxy)-phenyl]-5-methyl-pyrrol-1-yl}-6-(1,1-difluoro-methoxy)-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage QUAD 4 system on a 25S column eluting in 10% EtOAc/i-hex.

LC/MS t=4.19 [MH⁺] 542/544.

### d) 3-{2-[5-Bromo-2-benzyloxy)-phenyl]-5-methyl-pyrrol-1-yl}-6-(1,1-difluoro-methoxy)-benzoic acid

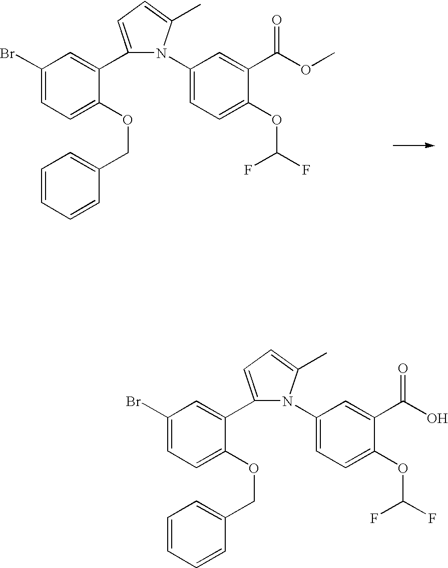

Procedure as for 3-{2-[5-bromo-2-(benzyloxy)-phenyl]-5-methyl-pyrrol-1-yl}-6-acetylamino-benzoic acid.

¹H-NMR (400 MHz, CDCl₃) 2.15 (3H, s), 4.72 (2H, s), 6.13 (1H, d, J=3 Hz), 6.30 (1H, d, J=3 Hz), 6.55 (1H, d, J=9 Hz), 6.59 (1H, t, J=75 Hz), 7.03-7.10 (4H, m), 7.22 (1H, dd, J=3 Hz, 9 Hz), 7.25-7.31 (3H+ CDCl₃, m), 7.40 (1H, d, J=3 Hz), 7.68 (1H, d, J=2 Hz).

LC/MS t=4.14 [MH⁺] 528/530.

### Example 178

### 3-{2-[5-Bromo-2-(benzyloxy)-phenyl]-5-methyl-pyrrol-1-yl}-5-amino-benzoic acid

### a) 3-{2-[5-Bromo-2-(benzyloxy)-phenyl]-5-methyl-pyrrol-1-yl}-5-amino-benzoic acid methyl ester

Procedure as for 3-{2-[5bromo-2-(benzyloxy)-phenyl]-5-methyl-pyrrol-1-yl}-6-acetylamino-benzoic acid methyl ester using the appropriate amine, however purification achieved using column chromatography on the Biotage QUAD 4 system on a 25S column eluting in 20% EtOAc/i-hex.

LC/MS t=3.68 min [MH⁺] 491/493.

### b) 3-{2-[5-Bromo-2-(benzyloxy)-phenyl]-5-methyl-pyrrol-1-yl}-5-amino-benzoic acid

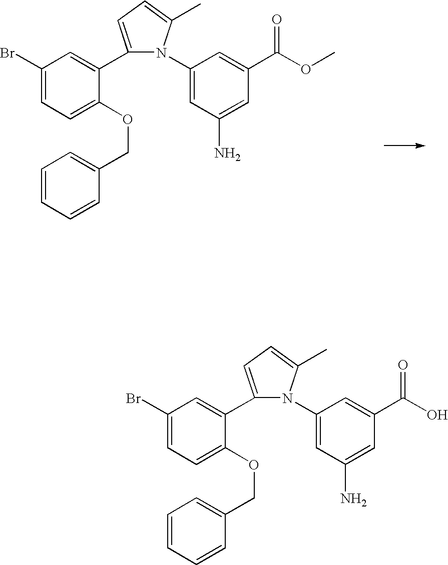

Procedure as for 3-{2-[5-bromo-2-(benzyloxy)-phenyl]-5-methyl-pyrrol-1-yl}-5-trifluoromethyl-benzoic acid.

¹H-NMR (400 MHz, MeOD) 2.12 (3H, s), 4.86 (2H, s), 5.99-6.02 (1H, m), 6.19-6.22 (1H, m), 6.44-6.46 (1H, m), 6.68-6.72 (1H, m), 7.02-7.05 (1H, m), 7.12-7.32 (8H, m).

LC/MS t=3.58 min [MH⁺] 477/479.

### Example 179

### 3-{2-[5-Bromo-2-benzyloxy-phenyl]-5-methyl-pyrrol-1-yl}-5-ethylamino-benzoic acid

### a) 3-{2-[5-Bromo-2-(benzyloxy)-phenyl]-5-methyl-pyrrol-1-yl}-ethylamino-benzoic acid methyl ester

3-{2-[5-Bromo-2-(benzyloxy)-phenyl]-5-methyl-pyrrol-1-yl}-5-amino-benzoic acid methyl ester (0.25g, 0.509 mmol), potassium carbonate (0.01g, 0.509 mmol), ethyl iodide (0.04 ml, 0.509 mmol) and NMP (3 ml) were heated in a sealed vessel using microwaves for 30 minutes. Upon cooling the reaction was diluted with Et₂O and washed with dilute citric acid, the organics were extracted and the aqueous washed with 2xEt₂O. The combined organics were then washed with 2×H₂O and brine, dried over MgSO₄, filtered and concentrated in vacuo. The residue was purified by chromatography on silica gel eluting with 10% EtOAc/iso-hexane. This yielded the title compound as a clear oil (0.036g, 14%).

LC/MS t=4.16 min [MH⁺] 519/521.

### b) 3-{2-[5-Bromo-2-(benzyloxy)-phenyl]-5-methyl-pyrrol-1-yl}-5-ethylamino-benzoic acid

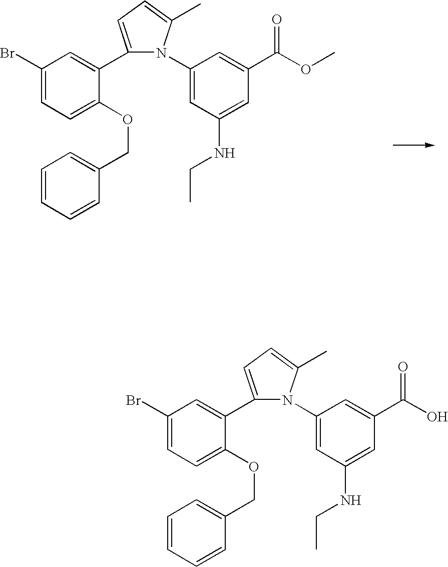

Procedure as for 3-[2-(5bromo-2-benzyloxy-phenyl)-5-methyl-pyrrol-1-yl]-5-trifluoromethyl-benzoic acid.

LC/MS t=3.79 min [MH⁺] 505/507.

### Example 180

### 3-{2-[5-Bromo-2-(benzyloxy)-phenyl]-5-methyl-pyrrol-1-yl}-5-(1,1-dioxo-1I⁶-isothiazolidin-2-yl)-benzoic acid

### a) 3-Amino-5-nitro-benzoic acid methyl ester

To a solution of 3-amino-5-nitro-benzoic acid (ex Avocado) (65 g, 357 mmol, 1 equiv) in MeOH (650 ml) at 0° C. was added SOCl₂ dropwise (39 ml, 536 mmol, 1.5 equiv). The resulting solution was allowed to warm to room temperature and stirred for 16 h. A further portion of SOCl₂ (10 ml, 137 mmol, 0.4 equiv) was added dropwise and the solution was stirred at room temperature for 5 h, at 50° C. for 2 h and then cooled to room temperature and concentrated in vacuo. The residue was dissolved in EtOAc and the organic phase washed with saturated NaHCO₃ solution, dried over MgSO₄ and concentrated in vacuo. The solid residue was triturated with EtOAc/iso-hexane to give the title compound (55 g, 78%).

### b) 3-(3-Chloro-propane-1-sulfonylamino)-5-nitro-benzoic acid methyl ester

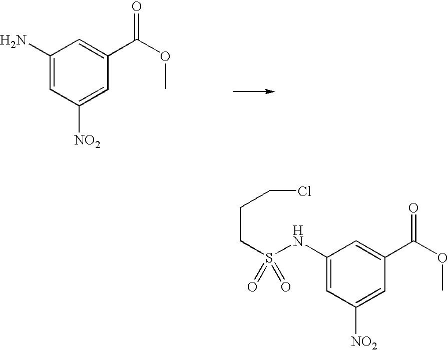

To a solution of 3-amino-5-nitro-benzoic acid methyl ester (45 g, 229 mmol, 1 equiv) in CH₂Cl₂ (450 ml) was added pyridine (18.5 ml, 229 mmol, 1 equiv), DMAP (100 mg, 0.8 mmol, catalytic) and 3-chloropropanesulfonyl chloride (28 ml, 230 mmol). The resulting mixture was stirred for 40 h then diluted with EtOAc. The organic phase was diluted with 2MN HCl. The resulting solid was filtered to give 3-(3-chloro-propane-1-sulfonylamino)-5-nitro-benzoic acid methyl ester (23 g, 32%). The filtrate was separated and the organic phase was washed with saturated aqueous NaHCO₃ solution, dried over MgSO₄ and concentrated in vacuo. The residue was triturated with EtOAc and iso-hexane to give a further 50 g (65%) of 3-(3-chloro-propane-1-sulfonylamino)-5-nitro-benzoic acid methyl ester, as a pale brown solid, which was used in the next step without further purification.

LC/MS t=3.11 min, [MH⁻]=335.

### c) 3-{1,1-Dioxo-1I⁶-isothiazolidin-2-yl)-5-nitro-benzoic acid methyl ester

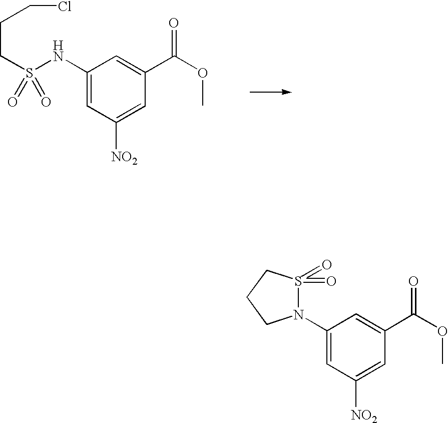

To a solution of 3-(3-chloro-propane-1-sulfonylamino)-5-nitro-benzoic acid methyl ester (73g, 217 mmol, 1 equiv) in EtOH (600 ml) was added Et₃N (60 ml, 430 mmol, 2 equiv) and the resulting mixture was refluxed for 3 h, cooled to room temperature and concentrated in vacuo. The residue was dissolved in EtOAc, washed with 2M HCl, dried over MgSO₄ and concentrated in vacuo. The residue was triturated with iso-hexane and EtOAc to give 3-(1,1-dioxo-1I⁶-isothiazolidin-2-yl)-5-nitro-benzoic acid methyl ester (58 g, 88%), as a pale brown solid, which was used in the next step without further purification.

LC/MS t=2.78 min [M+H+NH₃⁺]=318.0

### d) 3-Amino-6-(1,1-dioxo-1I⁶-isothiazoidin-2-yl)-benzoic acid methyl ester

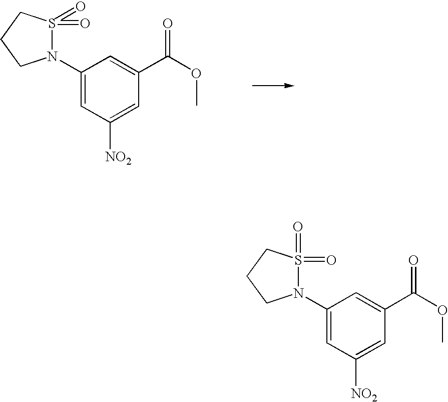

A flask was charged with 3-(1,1-dioxo-1I⁶-isothiazolidin-2-yl)-5-nitro-benzoic acid methyl ester (25 g, 83 mmol, 1 equiv) and 10% palladium (0) on charcoal (50% wet, 5 g, 10% w/w) and EtOH (500 ml). The resulting suspension was stirred under an atmosphere of hydrogen (atmospheric pressure) for 4 h after which time the catalyst was filtered off, through a pad of celite. The catalyst was washed three times with DMF and the combined organic layers were concentrated in vacuo. The residue was dissolved in EtOAc and filtered again through celite in order to remove residual catalyst. The organic phase was concentrated in vacuo. The residue was triturated with Et₂O to give 3-amino-5-(1,1-dioxo-1I⁶-isothiazolidin-2-yl)-benzoic acid methyl ester (18 g, 80%), as a pale brown solid, which was used in the next step without further purification.

LC/MS t=2.16 min [MH⁺]=271

### e) 3-{2-[5-Bromo-2-(benzyloxy)-phenyl]-5-methyl-pyrrol-1-yl}-5-(1,1-dioxo-1I⁶-isothiazolidin-2-yl)-benzoic acid methyl ester

Procedure as for 3-{2-[5-bromo-2-(benzyloxy)-phenyl]-5-methyl-pyrrol-1-yl}-6-acetylamino-benzoic acid methyl ester, however purification achieved using column chromatography on the Biotage® QUAD 4 system on a 25S column eluting in 50% EtOAc/i-hex.

LC/MS t=3.71 min [MH⁺] 595/597.

### f) 3-{2-[5-Bromo-2-benzyloxy)-phenyl]-5-methyl-pyrrol-1-yl}-5-(1,1-dioxo-1I⁶-isothiazolidin-2-yl)-benzoic acid

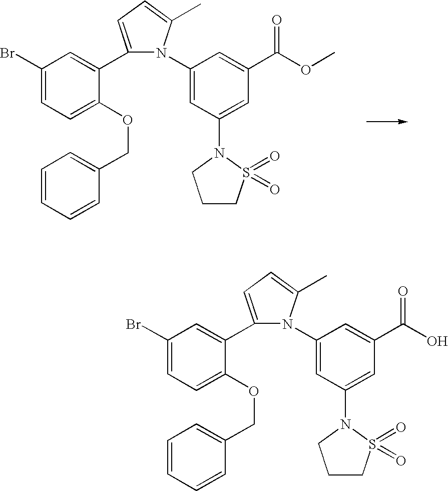

Procedure as for 3-{2-[5-bromo-2-(benzyloxy)-phenyl]-5-methyl-pyrrol-1-yl}-6-acetylamino-benzoic acid.

¹H-NMR (400 MHz, CDCl₃) 2.20 (3H, s), 2.45 (2H, m), 3.33 (2H, t, J=7 Hz), 3.43 (2H, t, J=7 Hz), 4.75 (2H, s), 6.13 (1H, d, J=3 Hz), 6.29 (1H, d, J=3 Hz), 6.53 (1H, d, J=9 Hz), 7.04 (2H, dd, J=1 Hz, 8 Hz), 7.10 (1H, t, J=2 Hz), 7.20 (1H, dd, J=2 Hz, 9 Hz), 7.22-7.30 (3H+ CDCl₃, m), 7.43 (1H, d, J=2 Hz), 7.49 (1H, t, J=1 Hz), 7.77-7.79 (1H, m).

LC/MS t=3.82 min [MH⁺] 581/583.

### Example 181

### 3-{2-[5-Bromo-2-(benzyloxy)-phenyl]-5-methyl-pyrrol-1-yl}-5-(2-oxo-pyrrolidin-1-yl)-benzoic acid

### a) 3-{2-[5-Bromo-2-(benzyloxy)-phenyl]-5-methyl-pyrrol-1-yl}-5-(2-oxo-pyrrolidin-1-yl)-benzoic acid methyl ester

Procedure as for 3-{2-[5bromo-2-(benzyloxy)-phenyl]-5-methyl-pyrrol-1-yl}-6-acetylamino-benzoic acid methyl ester using the appropriate amine, however purification achieved using column chromatography on the Biotage® QUAD 4 system on a 25S column eluting in 50% EtOAc/i-hex.

LC/MS t=3.75 min [MH⁺] 559/561.

### b) 3-{2-[5-Bromo-2-(benzyloxy)-phenyl]-5-methyl-pyrrol-1-yl}-5-(2-oxo-pyrrolidin-1-yl)-benzoic acid

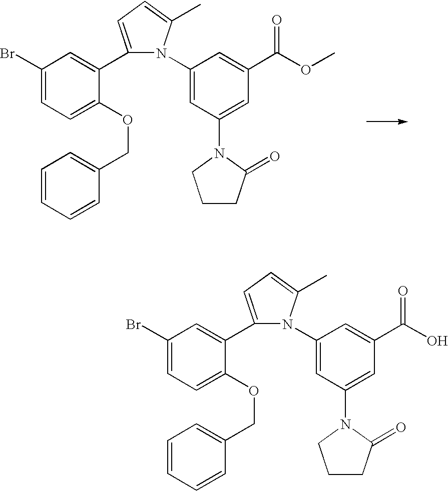

Procedure as for 3-{2-[5-bromo-2-(benzyloxy)-phenyl]-5-methyl-pyrrol-1-yl}-6-acetylamino-benzoic acid.

¹H-NMR (400 MHz, CDCl₃) 2.10 (2H, m), 2.20 (3H, s), 2.55 (2H, t, J=8 Hz), 3.52 (2H, m), 4.76 (2H, s), 6.13 (1H, d, J=3 Hz), 6.30 (1H, d, J=3 Hz), 6.52 (1H, d, J=9 Hz), 7.00-7.03 (2H, m), 7.19 (1H, dd, J=2 Hz, 9 Hz), 7.25-7.29 (3H+ CDCl₃, m), 7.42 (1H, d, J=3 Hz), 7.48-7.50 (1H, m), 7.56 (1H, t, J=2 Hz), 8.19-8.21 (1H, m).

LC/MS t=3.88 min [MH⁺] 545/547.

### Example 182

### 3-{2-[5-Bromo-2-(benzyloxy)-phenyl]-5-methyl-pyrrol-1-yl}-5-(2-oxo-piperidin-1-yl)-benzoic acid

### a) 3-Amino-5-(2-oxo-piperidin-1-yl)-benzoic acid methyl ester

Prepared in an analogous manner to 3-amino-5-(2-oxo-pyrrolidin-1-yl)-benzoic acid methyl ester using the appropriate amine.

### b) 3-{2-[5-Bromo-2-(benzyloxy)-phenyl]-5-methyl-pyrrol-1-yl}-5-(2-oxo-piperidin-1-yl)-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage® QUAD 4 system on a 25S column eluting in 50% EtOAc/i-hex.

LC/MS t=3.94 min [MH⁺] 573/575.

### c) 3-{2-[5-Bromo-2-(benzyloxy)-phenyl]-5-methyl-pyrrol-1-yl}-5-(2-oxo-piperidin-1-yl)-benzoic acid

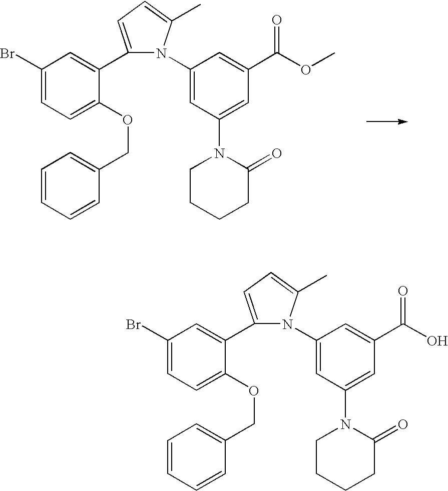

Procedure as for 3-{2-[5-bromo-2-(benzyloxy)-phenyl]-5-methyl-pyrrol-1-yl}-6-acetylamino-benzoic acid.

¹H-NMR (400 MHz, CDCl₃) 1.45-1.91 (4H, m), 2.15-2.22 (3H, m), 2.33-2.54 (2H, m), 2.89-3.33 (2H, m), 4.71-4.78 (2H, m), 6.09-6.14 (1H, m), 6.28-6.34 (1H, m), 6.51-6.55 (1H, m), 7.02-7.30 (7H, m), 7.36-7.41 (1H, m), 7.59 (1H, t, J=1 Hz), 7.87 (1H, t, J=1 Hz).

LC/MS t=3.61 min.

### Example 183

### 3-{2-[5-Bromo-2-benzyloxy)-phenyl]-5-methyl-pyrrol-1-yl}-5-amino-6-methyl-benzoic acid

### a) 3-{2-[5-Bromo-2-(benzyloxy)-phenyl]-5-methyl-pyrrol-1-yl}-5-amino-6-methyl-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved by column chromatography on the Biotage® QUAD 4 system on a 25S column eluting in 20% EtOAc/i-hex.

LC/MS t=3.75 min [MH⁺] 505/507.

### b) 3-{2-[5-Bromo-2-(benzyloxy)-phenyl]-5-methyl-pyrrol-1-yl}-5-amino-6-methyl-benzoic acid

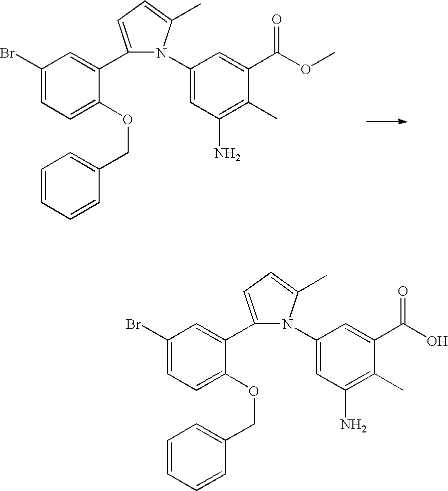

Procedure as for 3-{2-[5-bromo-2-(benzyloxy)-phenyl]-5-methyl-pyrrol-1-yl}-6-acetylamino-benzoic acid.

¹H-NMR (400 MHz, CDCl₃) 2.14 (3H, s), 2.37 (3H, s), 4.80 (2H, s), 6.09 (1H, d, J=3 Hz), 6.27 (1H, d, J=3 Hz), 6.41 (1H, d, J=2 Hz), 6.45 (1H, d, J=9 Hz), 7.07-7.11 (3H, m), 7.17 (1H, dd, J=2 Hz, 9 Hz), 7.25-7.32 (4H, m), 7.37 (1H, d, J=2 Hz).

LC/MS t=3.79 min [MH⁺] 491/493.

### Example 184

### 2-{2-[5-Bromo-2-(benzyloxy)-phenyl]-5-methyl-pyrrol-1-yl}-isonicotinic acid

### a) 2-{2-[5-Bromo-2-(benzyloxy)-phenyl]-5-methyl-pyrrol-1-yl}-isonicotinic acid ethyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved by column chromatography on a Biotage® 25M column eluting in 20% EtOAc/i-hex.

LC/MS t=4.11 min [MH⁺] 491/493.

### b) 2-{2-[5-Bromo-2-(benzyloxy)-phenyl]-5-methyl-pyrrol-1-yl}-isonicotinic acid

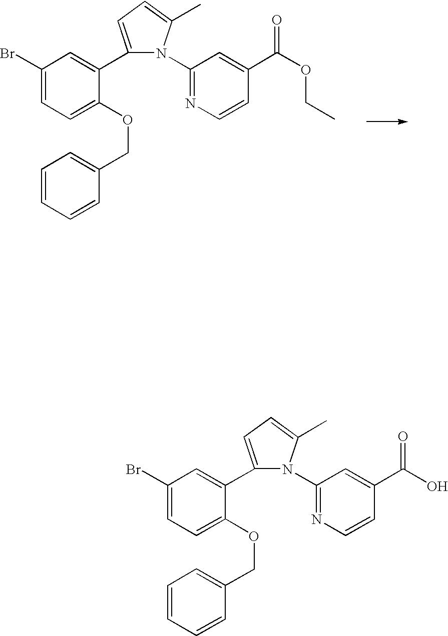

Procedure as for 3-{2-[5-bromo-2-(benzyloxy)-phenyl]-5-methyl-pyrrol-1-yl}-5-trifluoromethyl-benzoic acid.

LC/MS t=3.74 min [MH⁺] 463/465.

### Example 185

### 3-{2-[5-Bromo-2-(benzyloxy)-phenyl]-5-methyl-pyrrol-1-yl}-5-trifluoromethyl-benzoic acid

### a) 3-{2-[5-Bromo-2-(benzyloxy)-phenyl]-5-methyl-pyrrol-1-yl}-5-trifluoromethyl-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on a Biotage® 25S column eluting in 5% EtOAc/i-hex.

LC/MS t=4.40 min [MH⁺] 544/546.

### b) 3-{2-[5-Bromo-2-(benzyloxy)-phenyl]-5-methyl-pyrrol-1-yl}-5-trifluoromethyl-benzoic acid

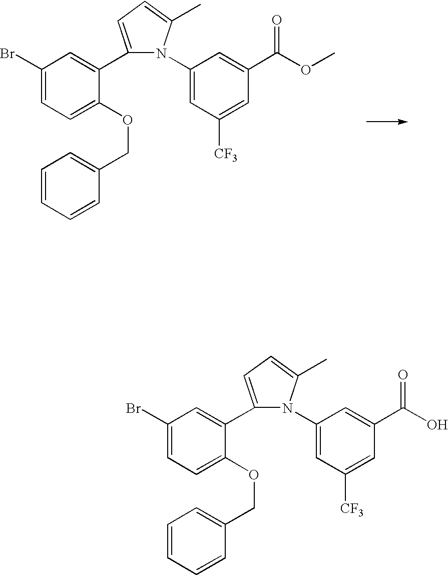

3-{2-[5-Bromo-2-(benzyloxy)-phenyl]-5-methyl-pyrrol-1-yl}-5-trifluoromethyl-benzoic acid ethyl ester (0.06g) was dissolved in EtOH (2 ml) and stirred with 2N NaOH (1 ml) at 120° C. in a sealed vessel for 3 minutes using microwaves. The solvent was removed in vacuo, the residue taken up in DCM and washed with dil. citric acid. The organics were separated using a phase separator column with a NaSO₄ cartridge attached. The solvent was removed in vacuo and the residue freeze-dried in a MeCN/H₂O solution to yield a yellow solid (0.049g, 86%).

¹H-NMR (400 MHz, CDCl₃) 2.17 (3H, s), 4.69 (2H, s), 6.17 (1H, d, J=3 Hz), 6.32 (1H, d, J=4 Hz), 6.52 (1H, d, J=9 Hz), 6.98-7.03 (2H, m), 7.22 (1H, dd, J=3 Hz, 9 Hz), 7.23-7.31 (3H+ CDCl₃, m), 7.41-7.45 (2H, m), 7.86-7.88 (1H, m), 8.17 (1H, s).

LC/MS t=4.05 min [MH⁺] 530/532.

### Example 186

### 3-{2-[5-Bromo-2-(benzyloxy)-phenyl]-5-methyl-pyrrol-1-yl}-6-chloro-benzoic acid

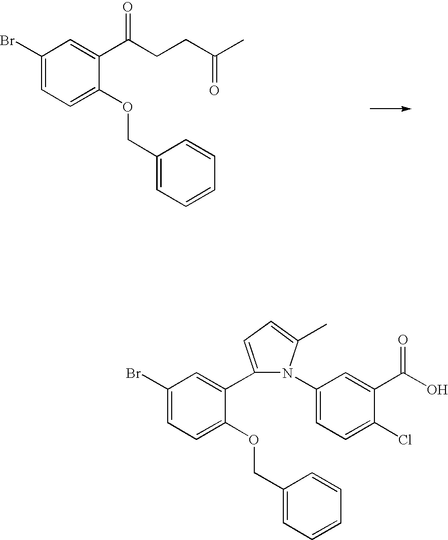

1-[2-(Benzyloxy)-5-bromo-phenyl]-pentane-1,4-dione (0.12g, 0.322 mmol), 5-amino-2-chloro-benzoic acid (0.062g, 0.365 mmol) and p-TSA (cat.) in NMP (2 ml) were heated in a sealed vessel at 150° C. for 10 minutes using microwaves. Upon cooling the reaction was diluted with Et₂O and washed with dil. citric acid. The organic layer was extracted and the aqueous layer washed with 3xEt₂O, the combined organic extracts were then washed with brine, dried over MgSO₄, filtered and concentrated in vacuo. The residue was purified by MDAP. This yielded the title compound as a white solid (0.04g, 24%).

¹H-NMR (400 MHz, CDCl₃) 2.15 (3H, s), 4.72 (2H, s), 6.14 (1H, dd, J=1 Hz, 3 Hz), 6.29 (1H, d, J=3 Hz), 6.54 (1H, d, J=9 Hz), 6.97-7.03 (3H, m), 7.21-7.31 (5H, m), 7.44 (1H, d, J=3 Hz), 7.65 (1H, d, J=3 Hz).

LC/MS t=4.32 min [MH⁺] 496/498.

### Example 187

### 3-{2-[5-Bromo-2-(benzyloxy)-phenyl]-5-methyl-pyrrol-1-yl}-5-acetylamino-benzoic acid

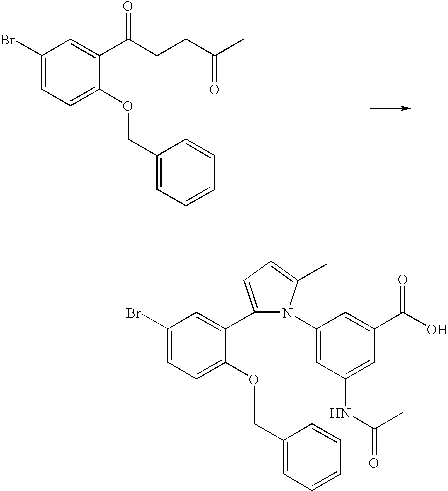

Procedure as for 3-{2-[5-bromo-2-(benzyloxy)-phenyl]-5-methyl-pyrrol-1-yl}-6-chloro-benzoic acid using the appropriate amine, however purification achieved using column chromatography on a SPE cartridge (25 g) eluting in 5% MeOH/DCM.

¹H-NMR (400 MHz, MeOD) 2.07 (3H, s), 2.13 (3H, s), 4.78 (2H, s), 6.06 (1H, d, J=3 Hz), 6.21 (1H, d, J=3 Hz), 6.65 (1H, d, J=9 Hz), 7.09 (2H, d, J=7 Hz), 7.18 (1H, dd, J=2 Hz, 9 Hz), 7.22-7.31 (4H, m), 7.35 (1H, s), 7.51 (1H, s), 8.02 (1H, s).

LC/MS t=3.71 min [MH⁻] 517/519.

### Example 188

### 3-{2-[5-Bromo-2-(benzyloxy)-phenyl]-5-methyl-pyrrol-1-yl}-6-methyl-benzoic acid

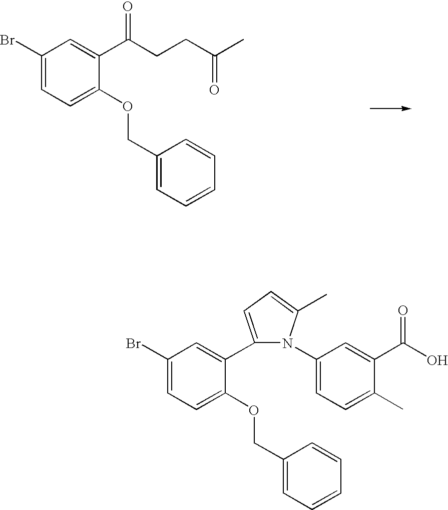

Procedure as for 3-{2-[5-bromo-2-(benzyloxy)-phenyl]-5-methyl-pyrrol-1-yl}-6-chloro-benzoic acid using the appropriate amine, however purification achieved using column chromatography on a SPE cartridge (25 g) eluting in 20% EtOAc/i-hex.

¹H-NMR (400 MHz, CDCl₃) 2.15 (3H, s), 2.62 (3H, s), 4.77 (2H, s), 6.12 (1H, d, J=3 Hz), 6.30 (1H, d, J=2 Hz), 6.52 (1H, d, J=9 Hz), 7.00-7.04 (3H, m), 7.11 (1H, d, J=8 Hz), 7.18 (1H, dd, J=2 Hz, 9 Hz), 7.22-7.29 (3H+ CDCl₃, m), 7.38 (1H, d, J=3 Hz), 7.77 (1H, d, J=2 Hz).

LC/MS t=4.11 min [MH⁺] 476/478.

### Example 189

### 3-{2-[5-Bromo-2-(benzyloxy)-phenyl]-5-methyl-pyrrol-1-yl}-6-fluoro-benzoic acid

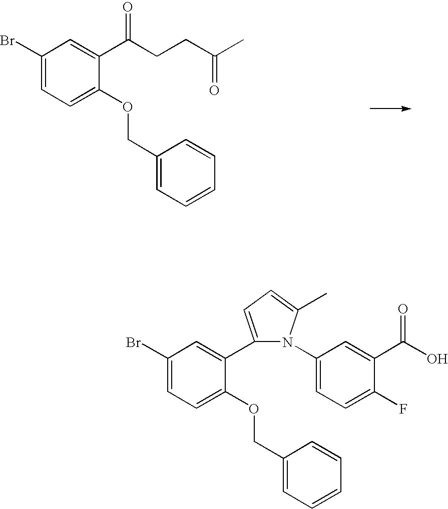

Procedure as for 3-{2-[5-bromo-2-(benzyloxy)-phenyl]-5-methyl-pyrrol-1-yl}-6-chloro-benzoic acid using the appropriate amine, however purification achieved using column chromatography on a SPE cartridge (10g) eluting in 20% EtOAc/i-hex.

¹H-NMR (400 MHz, CDCl₃) 2.13 (3H, s), 4.75 (2H, s), 6.12 (1H, d, J=3 Hz), 6.29 (1H, d, J=3 Hz), 6.55 (1H, d, J=9 Hz), 6.95-7.01 (1H, m), 7.04-7.12 (3H, m), 7.22 (1H, dd, J=3 Hz, 9 Hz), 7.25-7.32 (3H+ CDCl₃, m), 7.40 (1H, d, J=3 Hz), 7.68 (1H, dd, J=3 Hz, 7 Hz).

LC/MS t=4.00 min [MH⁺] 480/482.

### Example 190

### 3-{2-[5-Bromo-2-(benzyloxy)-phenyl]-5-methyl-pyrrol-1-yl}-6-hydroxy-benzoic acid

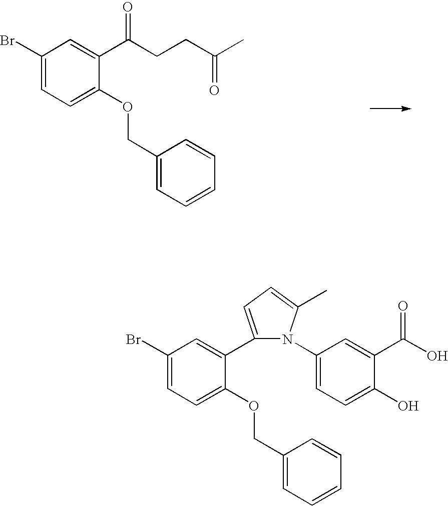

Procedure as for 3-{2-[5-bromo-2-(benzyloxy)-phenyl]-5-methyl-pyrrol-1-yl}-6-chloro-benzoic acid using the appropriate amine, however purification achieved using the MDAP.

¹H-NMR (400 MHz, CDCl₃) 2.12 (3H, s), 4.80 (2H, s), 6.11 (1H, d, J=3 Hz), 6.28 (1H, d, J=3 Hz), 6.54 (1H, d, J=9 Hz), 6.83 (1H, d, J=9 Hz), 7.05-7.10 (3H, m), 7.19 (1H, d, J=3 Hz, 9 Hz), 7.25-7.32 (3H+ CDCl₃, m), 7.37 (1H, d, J=2 Hz), 7.56 (1H, d, J=2 Hz), 10.51 (1H, broad s).

LC/MS t=4.28 min [MH⁺] 478/480.

### Example 191

### 3-{2-[5-Bromo-2-(benzyloxy)-phenyl]-5-methyl-pyrrol-1-yl}-6-methoxy-benzoic acid

### a) 3-{2-[5-Bromo-2-(benzyloxy)-phenyl]-5-methyl-pyrrol-1-yl}-6-methoxy-benzoic acid methyl ester

3-{2-[5-Bromo-2-(benzyloxy)-phenyl]-5-methyl-pyrrol-1-yl}-6-hydroxy-benzoic acid (0.069g, 0.144 mmol), sodium hydride (0.048g, 2 mmol) in anhydrous DMF were stirred at 0° C. for 2 hours, methyl iodide (0.1 ml, 1.44 mmol) was added and the mixture was stirred under a nitrogen atmosphere for 3 hours. The solvent was removed in vacuo and the residue taken up in EtOAc and washed with 2×H₂O and brine. The organics were then dried over MgSO₄ and the solvent removed in vacuo. The residue was purified by column chromatography, using a SPE cartridge (10g) eluting with 20% EtOAc/iso-hexane. This yielded the title compound as a clear oil (0.037g, 52%).

LC/MS t=3.81 min [MH⁺] 506/508.

### b) 3-{2-[5-Bromo-2-(benzyloxy)-phenyl]-5-methyl-pyrrol-1-yl}-6-methoxy-benzoic acid

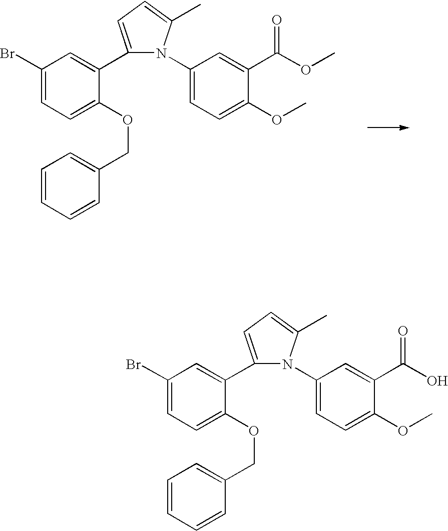

Procedure as for 3-{2-[5-bromo-2-(benzyloxy)-phenyl]-5-methyl-pyrrol-1-yl}-5-trifluoromethyl-benzoic acid.

¹H-NMR (400 MHz, CDCl₃) 2.12 (3H, s), 4.04 (3H, s), 4.83 (2H, s), 6.11 (1H, d, J=3 Hz), 6.28 (1H, d, J=3 Hz), 6.55 (1H, d, J=9 Hz), 6.88 (1H, d, J=9 Hz), 7.06-7.09 (2H, m), 7.12-7.18 (2H, m), 7.25-7.32 (4H+ CDCl₃, m), 7.97 (1H, d, J=3 Hz).

LC/MS t=3.65 min [MH⁺] 492/494.

### Example 192

### 3-{2-[5-Bromo-2-(benzyloxy)-phenyl]-5-methyl-pyrrol-1-yl}-1-naphthoic acid

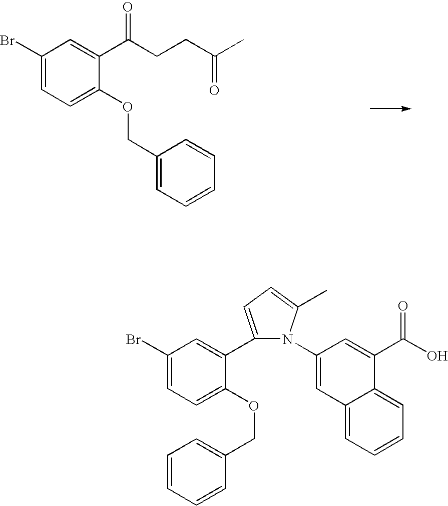

Procedure as for 3-{2-[5-bromo-2-(benzyloxy)-phenyl]-5-methyl-pyrrol-1-yl}-6-chloro-benzoic acid using the appropriate amine, however purification achieved by column chromatography using a SPE cartridge (10g) eluting in 30% EtOAc/i-hex.

LC/MS t=3.95 min [MH⁺] 512/514.

### Example 193

### 3-{2-[5-Bromo-2-(benzyloxy)-phenyl]-5-methyl-pyrrol-1-yl}-4-fluoro-benzoic acid

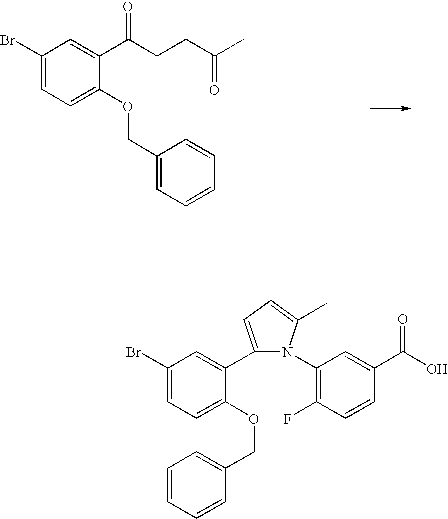

Procedure as for 3-{2-[5bromo-2-(benzyloxy)-phenyl]-5-methyl-pyrrol-1-yl}-6-chloro-benzoic acid using the appropriate amine, however purification achieved by column chromatography on a Biotage® 15M column eluting in 30% EtOAc/i-hex.

LC/MS t=3.79 min [MH⁺] 480/482.

### Example 194

### 3-{2-[5-Bromo-2-(benzyloxy)-phenyl]-5-methyl-pyrrol-1-yl}-5-propylamino-benzoic acid

### a) 3-Nitro-5-(propylamino) benzoic acid

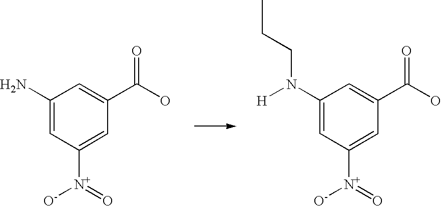

3-Amino-5-nitrobenzoic acid(500 mg, 2.7 mmol) in 2-butanone (2 ml) with potassium carbonate (500 mg) was treated with 1-bromopropane (1 ml, exess). The reaction mixture was then heated under reflux, under nitrogen for three hours, filtered and the residue washed with EtOAc (10 ml).The organic layer was then washed with water (2×10 ml), dried over magnesium sulphate, and chromatographed on a Water's sep-pack(10 g) giving the title compound (350 mg, 58%).

LC/MS t=3.23 min

### b) 3-Amino-5-(propylamino)benzoic acid

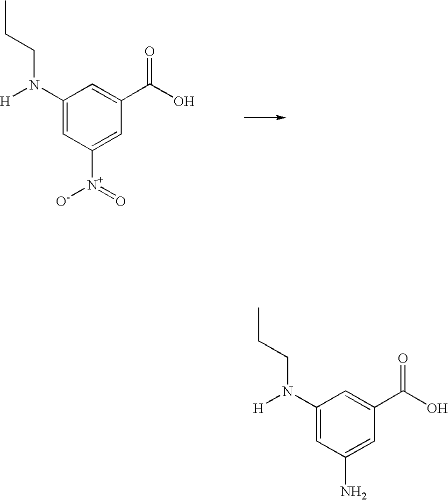

3-Nitro-5-(propylamino) benzoic acid (350 mg, 1.5 mmol) in methanol (15 ml) and palladium on charcoal 5% wet, was hydrogenated at atmospheric temperature and pressure for 4 hours. The reaction mixture was then filtered through high-flo and evaporated down to give the title compound (290 mg, 100%).

LC/MS t=2.14 min [MH⁺] 195

### c) 3-{2-[5-Bromo-2-(benzyloxy)-phenyl]-5-methyl-pyrrol-1-yl}-5-propylamino-benzoic acid

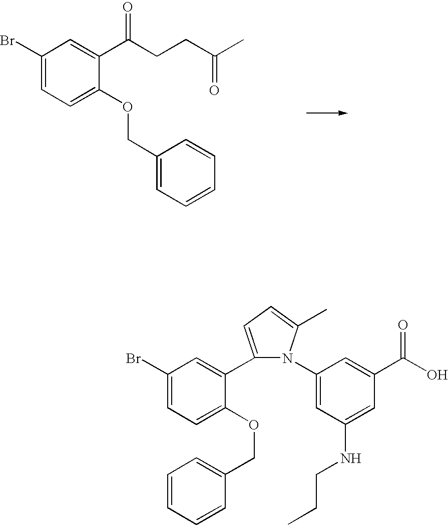

Procedure as for 3-{2-[5-bromo-2-(benzyloxy)-phenyl]-5-methyl-pyrrol-1-yl}-6-acetylamino-benzoic acid methyl ester using the appropriate amine, however further purification was achieved using the MDAP.

LC/MS t=3.91 min [MH⁺] 519/521.

### Example 195

### 3-{2-[5-Bromo-2-(2,4,6-trifluoro-benzyloxy)-phenyl]-5-methyl-pyrrol-1-yl}-6-(1,1-difluoro-methoxy)-benzoic acid

### a) 5-Bromo-2-(2,4,6-trifluoro-benzyloxy)-benzaldehyde

5-Bromo-2-hydroxy-benzaldehyde (4 g, 19.8 mmol), 2,4,6-trifluoro-benzyl bromide (5g, 21.9 mmol) and potassium carbonate (5.5g, 39.8 mmol) were stirred in DMF (75 ml) at 55° C. under a nitrogen atmosphere overnight. Upon cooling the reaction mixture was diluted with EtOAc and washed with H₂O. The organic layer was extracted and the aqueous layer washed with 3×EtOAc. The combined organic extracts were then washed with brine and dried over MgSO₄, filtered and concentrated in vacuo to yield the title compound as a white solid (6.95g, 100%).

LC/MS t=3.50 min.

### b) 1-[5-Bromo-2-(2,4,6-trifluoro-benzyloxy)-phenyl]-pentane-1,4-dione

2-(2,4,6-Trifluoro-benzyloxy)-5-bromo-benzaldehyde (6.95g, 20.15 mmol), triethylamine (8.4 ml, 60.43 mmol), methyl vinyl ketone (1.71 ml, 20.55 mmol) and 3-ethyl-5-(2-hydroxyethyl)-4-ethylthiazolium bromide (1.52g, 6.04 mmol) were stirred at refluxed in EtOH (1 5 ml) under a nitrogen atmosphere for 21 hours. Upon cooling the reaction mixture was diluted with EtOAc and washed with saturated NH₄Cl. The organic layer was extracted and the aqueous layer washed with 3×EtOAc. The combined organic extracts were then washed with saturated NaHCO₃ and brine and then dried over MgSO₄, filtered and concentrated in vacuo. The residue was purified by chromatography on silica gel eluting with 20% EtOAc/iso-hexane. This yielded the title compound as an off white solid (5.77g, 70%).

LC/MS t=3.58 min [MH⁺] 415/417.

### c) 3-{2-[5-Bromo-2-(2,4,6-trifluoro-benzyloxy)-phenyl]-5-methyl-pyrrol-1-yl}-6-(1,1-difluoro-methoxy)-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage® Horizon system on a 25S column eluting in a gradient of 520% EtOAc/i-hex.

LC/MS t=4.09 min [MH⁺] 596/598.

### d) 3-{2-[6-Bromo-2-(2,4,6-trifluoro-benzyloxy)-phenyl]-5-methyl-pyrrol-1-yl}-6-(1,1-difluoro-methoxy)-benzoic acid

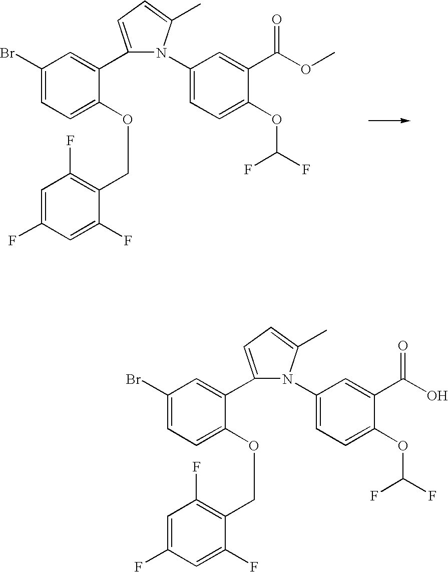

Procedure as for 3-[2-(5-bromo-2-benzyloxy-phenyl)-5-methyl-pyrrol-1-yl]-5-trifluoromethyl-benzoic acid, however further purification was required using column chromatography on the Biotage® Horizon system on a 25S reverse phase column eluting in a gradient of 30-100% MeCN/H₂O.

¹H-NMR (400 MHz, CDCl₃) 2.14 (3H, s), 4.73 (2H, s), 6.07 (1H, d, J=3 Hz), 6.21 (1H, d, J=3 Hz), 6.59 (1H, t, J=75 Hz), 6.62-6.69 (2H, m), 6.74 (1H, d, J=9 Hz), 7.08-7.16 (2H, m), 7.25-7.32 (2H+ CDCl₃, m), 7.71 (1H, d, J=2 Hz).

LC/MS t=3.79 min [MH⁺] 582/584.

### Example 196

### 3-{2-[5-Bromo-2-(2,4,6-trifluoro-benzyloxy)-phenyl]-5-methyl-pyrrol-1-yl}-5-trifluoromethyl-benzoic acid

### a) 3-{2-[5-Bromo-2-(2,4,6-trifluoro-benzyloxy)-phenyl]-5-methyl-pyrrol-1-yl}-trifluoromethyl-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage® Horizon system on a 25S column eluting in a gradient of 5-20% EtOAc/i-hex.

LC/MS t=4.28 min [MH⁺] 598/600.

### b) 3-{2-[5-Bromo-2-(2,4,6-trifluoro-benzyloxy)-phenyl]-5-methyl-pyrrol-1-yl}-trifluoromethyl-benzoic acid

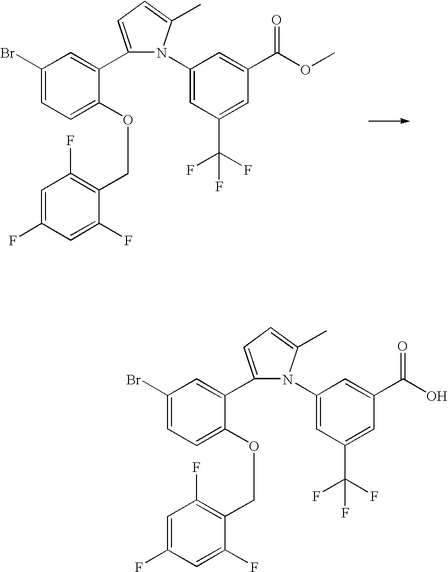

Procedure as for 3-{2-[5-bromo-2-(benzyloxy)-phenyl]-5-methyl-pyrrol-1-yl}-5-trifluoromethyl-benzoic acid.

¹H-NMR (400 MHz, CDCl₃) 2.16 (3H, s), 4.71 (2H, s), 6.10 (1H, d, J=3 Hz), 6.22 (1H, d, J=3 Hz), 6.62 (2H, t, J=9 Hz), 6.73 (1H, d, J=9 Hz), 7.28 (1H, dd, J=2 Hz, 9 Hz), 7.34 (1H,d, J=3 Hz), 7.42 (1H, s), 7.90 (1H, s), 8.21 (1H,s).

LC/MS t=3.98 min [MH⁺] 584/586.

### Example 197

### 3-{2-[5-Bromo-2-(2,4,6-trifluoro-benzyloxy)-phenyl]-5-methyl-pyrrol-1-yl}-5-amino-benzoic acid

### a) 3-{2-[5-Bromo-2-(2,4,6-trifluoro-benzyloxy)-phenyl]-5-methyl-pyrrol-1-yl}-5-amino-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage® Horizon system on a 25S column eluting in a gradient of 20-60% EtOAc/i-hex.

LC/MS t=3.89 min [MH⁺] 545/547.

### b) 3-{2-[5-Bromo-2-(2,4,6-trifluoro-benzyloxy)-phenyl]-5-methyl-pyrrol-1-yl}-5-amino-benzoic acid

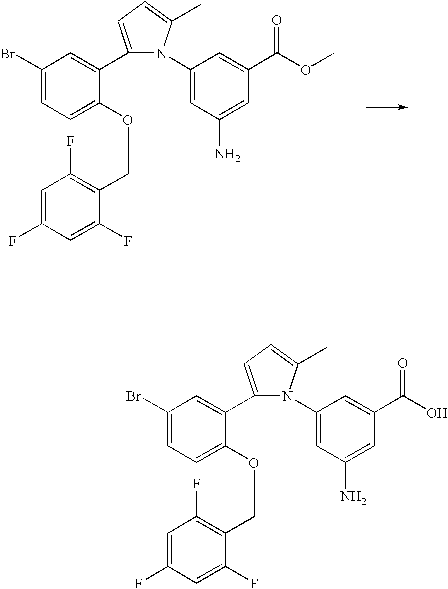

Procedure as for 3-{2-[5-bromo-2-(benzyloxy)-phenyl]-5-methyl-pyrrol-1-yl}-5-trifluoromethyl-benzoic acid.

¹H-NMR (400 MHz, CDCl₃) 2.13 (3H, s), 4.81 (2H, s), 4.90-6.00 (2H, broad s), 6.03 (1H, d, J=3 Hz), 6.25 (1H, d, J=3 Hz), 6.47 (1H, t, J=1 Hz), 6.65 (2H, t, J=8 Hz), 6.77 (1H, d, J=9 Hz), 7.19-7.25 (3H, m), 7.28-7.31 (1H, m), LC/MS t=3.54 min [MH⁺] 531/533.

### Example 198

### 3-{2-[5-Bromo-2-(2,4,6-trifluoro-benzyloxy)-phenyl]-5-methyl-pyrrol-1-yl}-5-(1,1-dioxo-1I⁶-isothiazolidin-2-yl)-benzoic acid

### a) 3-{2-[5-Bromo-2-(2,4,6-trifluoro-benzyloxy)-phenyl]-5-methyl-pyrrol-1-yl}-5-(1,1-dioxo-1I⁶-isothiazolidin-2-yl)-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage® Horizon system on a 25S column eluting in a gradient of 20-60% EtOAc/i-hex.

LC/MS t=3.89 min [MH⁺] 649/651.

### b) 3-{2-[5-Bromo-2-(2,4,6-trifluoro-benzyloxy)-phenyl]-5-methyl-pyrrol-1-yl}-5-(1,1-dioxo-1I⁶-isothiazolidin-2-yl)-benzoic acid

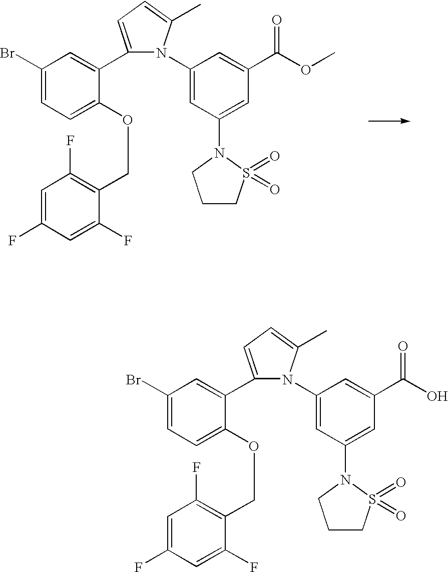

Procedure as for 3-{2-[5-bromo-2-(benzyloxy)-phenyl]-5-methyl-pyrrol-1-yl}-5-trifluoromethyl-benzoic acid, however further purification was required using column chromatography on the Biotage® Horizon system on a 25S reverse phase column eluting in a gradient of 30-100% MeCN/H₂O.

¹H-NMR (400 MHz, CDCl₃) 2.18 (3H, s), 2.45-2.55 (2H, m), 3.36 (2H, t, J=8 Hz), 3.60 (2H, t, J=7 Hz), 4.76 (2H, s), 6.05 (1H, d, J=3 Hz), 6.21 (1H, d, J=3 Hz), 6.63 (2H, t, J=8 Hz), 6.75 (1H, d, J=9 Hz), 7.11-7.14 (1H, m), 7.25-7.30 (1H+ CDCl₃, m), 7.32 (1H, d, J=3 Hz), 7.50-7.52 (1H, m), 7.77 (1H, m).

LC/MS t=3.56 min [MH⁺] 635/637.

### Example 199

### 3-{2-[5-Bromo-2-(2,4,6-trifluoro-benzyloxy)-phenyl]-5-methyl-pyrrol-1-yl}-5-(2-oxo-pyrrolidin-1-yl)-benzoic acid

### a) 3-{2-[5-Bromo-2-(2,4,6-trifluoro-benzyloxy)-phenyl]-5-methyl-pyrrol-1-yl}-5-(2-oxo-pyrrolidin-1-yl)-benzoic acid methyl ester

Procedure as for 3-{2-[5-bromo-2-(benzyloxy)-phenyl]-5-methyl-pyrrol-1-yl}-6-acetylamino-benzoic acid methyl ester using the appropriate amine, however purification achieved using column chromatography on the Biotage® Horizon system on a 25S column eluting in a gradient of 20-60% EtOAc/i-hex.

LC/MS t=3.92 min [MH⁺] 613/615.

### b) 3-{2-[5-Bromo-2-(2,4,6-trifluoro-benzyloxy)-phenyl]-5-methyl-pyrrol-1-yl}-5-(2-oxo-pyrrolidin-1-yl)-benzoic acid

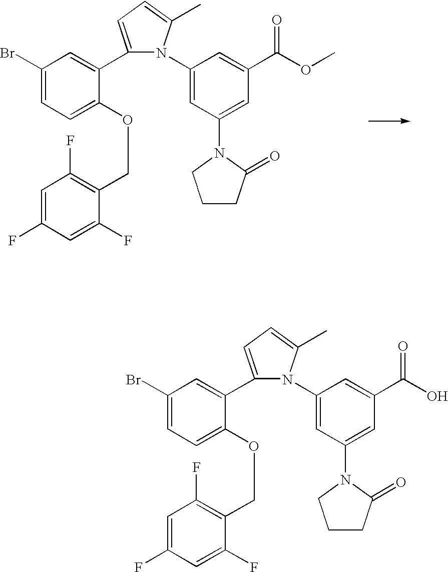

Procedure as for 3-{2-[5-bromo-2-(benzyloxy)-phenyl]-5-methyl-pyrrol-1-yl}-5-trifluoromethyl-benzoic acid.

¹H-NMR (400 MHz, d6-DMSO) 2.06 (3H, s), 2.47-2.55 (4H, m), 3.68-3.76 (2H, m), 4.79 (2H, s), 6.00-6.04 (1H, m), 6.15 (1H, d, J=3 Hz), 7.01 (1H, d, J=9 Hz), 7.13-7.25 (4H, m), 7.37 (1H, d, J=9 Hz), 7.57 (1H, s), 8.12 (1H, s).

LC/MS t=3.60 min [MH⁺] 599/601.

### Example 200

### 3-{2-[5-Bromo-2-(2,4,6-trifluoro-benzyloxy)-phenyl]-5-methyl-pyrrol-1-yl}-5-amino-6-methyl-benzoic acid

### a) 3-{2-[5-Bromo-2-(2,4,6-trifluoro-benzyloxy)-phenyl]-5-methyl-pyrrol-1-yl}-5-amino-6-methyl-benzoic acid methyl ester

Procedure as for 3-{2-[5-bromo-2-(benzyloxy)-phenyl]-5-methyl-pyrrol-1-yl}-6acetylamino-benzoic acid methyl ester using the appropriate amine, however purification achieved using column chromatography on the Biotage® Horizon system on a 25S column eluting in a gradient of 20-60% EtOAc/i-hex.

LC/MS t=3.93 min [MH⁺] 559/561.

### b) 3-{2-[5-Bromo-2-(2,4,6-trifluoro-benzyloxy)-phenyl]-5-methyl-pyrrol-1-yl}-5-amino-6-methyl-benzoic acid

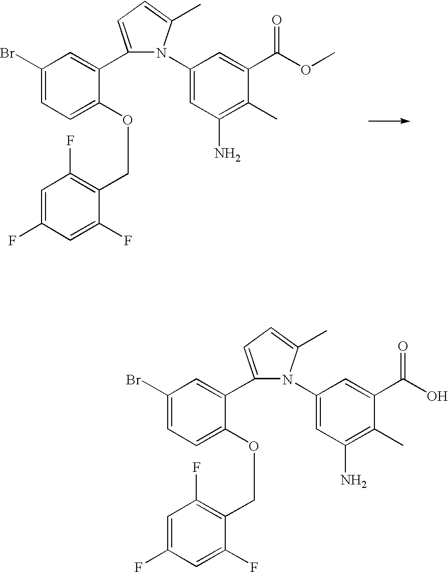

Procedure as for 3-{2-[5-bromo-2-(benzyloxy)-phenyl]-5-methyl-pyrrol-1-yl}-5-trifluoromethyl-benzoic acid, however further purification was required using column chromatography on the Biotage® Horizon system on a 25S reverse phase column eluting in a gradient of 30-100% MeCN/H₂O.

¹H-NMR (400 MHz, CDCl₃) 2.12 (3H, s), 2.39 (3H, s), 4.82 (2H, s), 6.02 (1H, d, J=3 Hz), 6.22 (1H, d, J=3 Hz), 6.45 (1H, s), 6.61-6.71 (2H, m), 6.77 (1H, d, J=9 Hz), 7.17 (1H, s), 7.19-7.32 (2H+ CDCl₃, m).

LC/MS t=3.55 min [MH⁺] 545/547.

### Example 201

### 3-{2-[5-Bromo-2-(2,4,6-trifluoro-benzyloxy)-phenyl]-5-methyl-pyrrol-1-yl}-6-fluoro-benzoic acid

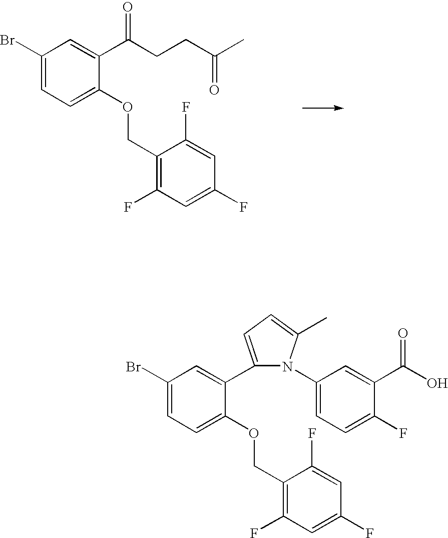

Procedure as for 3-{2-[5-bromo-2-(benzyloxy)-phenyl]-5-methyl-pyrrol-1-yl}-6-chloro-benzoic acid using the appropriate amine, however purification achieved using column chromatography on the Biotage® QUAD 4 system on a 25S column eluting in 25% EtOAc/i-hex (+AcOH).

¹H-NMR (400 MHz, CDCl₃) 2.12 (3H, s), 4.77 (2H, s), 6.07 (1H, d, J=3 Hz), 6.20 (1H, d, J=3 Hz), 6.65 (2H, t, J=9 Hz), 6.74 (1H, d, J=9 Hz), 6.99-7.06 (1H, m), 7.08-7.14 (1H, m), 7.25-7.32 (2H+ CDCl₃, m), 7.68-7.72 (1H, m).

LC/MS t=3.78 min [MH⁺] 534/536.

### Example 202

### 3-{2-[6-Bromo-2-(2,4,6-trifluoro-benzyloxy)-phenyl]-5-methyl-pyrrol-1-yl}-6-hydroxy-benzoic acid

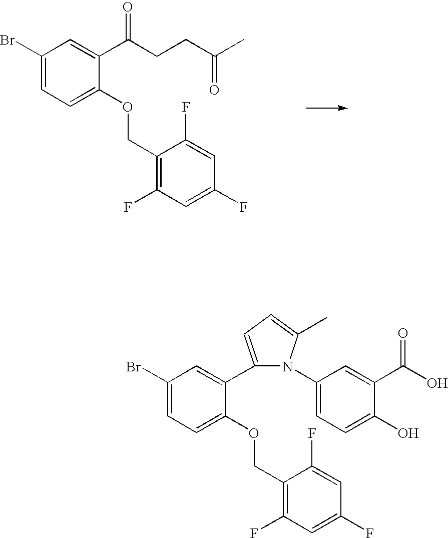

Procedure as for 3-{2-[5-bromo-2-(benzyloxy)-phenyl]-5-methyl-pyrrol-1-yl}-6-chloro-benzoic acid, however purification achieved using column chromatography on the Biotage® QUAD 4 system on a 25S column eluting in 40% EtOAc/i-hex (+AcOH).

¹H-NMR (400 MHz, MeOD) 2.05 (3H, s), 4.81 (2H, s), 5.96 (1H, d, J=3 Hz), 6.08 (1H, d, J=3 Hz), 6.78 (1H, d, J=9 Hz), 6.81-6.91 (4H, m), 6.99 (1H, dd, J=2 Hz, 9 Hz), 7.23 (1H, s), 7.29 (1H, dd, J=2 Hz, 9 Hz), 7.42 (1H, d, 2 Hz).

LC/MS t=4.18 min [MH⁺] 532/534.

### Example 203

### 3-{2-[5-Bromo-2-(2,4,6-trifluoro-benzyloxy)-phenyl]-5-methyl-pyrrol-1-yl}-naphthalene-1-carboxylic acid

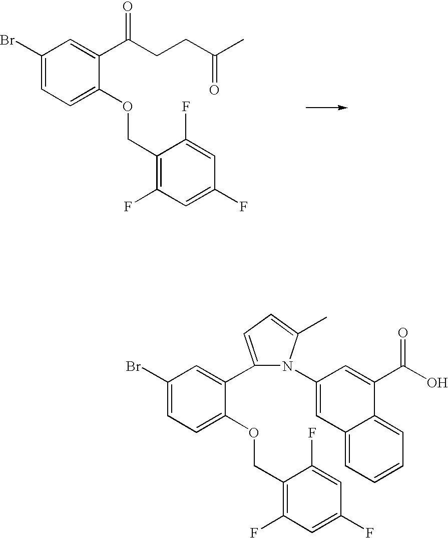

Procedure as for 3-{2-[5-bromo-2-(benzyloxy)-phenyl]-5-methyl-pyrrol-1-yl}-6-chloro-benzoic acid using the appropriate amine, however purification achieved using column chromatography on the Biotage® QUAD 4 system on a 25S column eluting in 25% EtOAc/i-hex (+AcOH).

LC/MS t=3.95 min [MH⁺] 566/568.

### Example 204

### 3-{2-[5-Bromo-2-(2,4,6-trifluoro-benzyloxy)-phenyl]-5-methyl-pyrrol-1-yl}-4-fluoro-benzoic acid

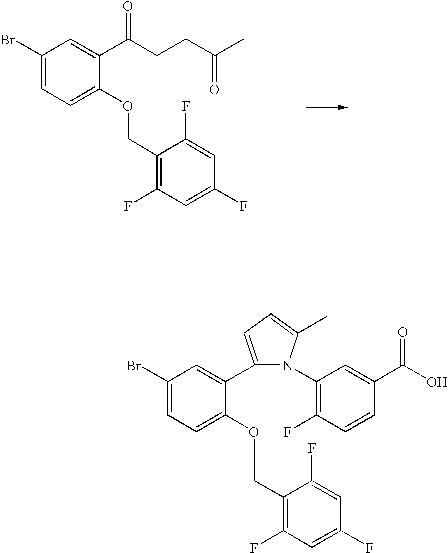

Procedure as for 3-{2-[5-bromo-2-(benzyloxy)-phenyl]-5-methyl-pyrrol-1-yl}-6-chloro-benzoic acid using the appropriate amine, however purification achieved using column chromatography on the Biotage® QUAD 4 system on a 25S column eluting in 25% EtOAc/i-hex (+AcOH).

LC/MS t=3.75 min [MH⁺] 534/536.

### Example 205

### 3-{2-[5-Bromo-2-(2,4,6-trifluoro-benzyloxy)-phenyl]-5-methyl-pyrrol-1-yl}-6-chloro-benzoic acid

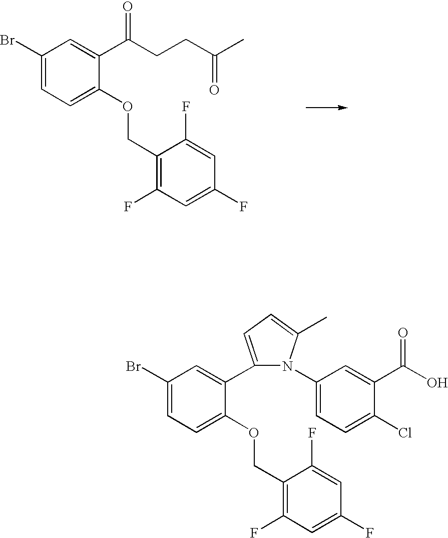

Procedure as for 3-{2-[5-bromo-2-(benzyloxy)-phenyl]-5-methyl-pyrrol-1-yl}-6-chloro-benzoic acid using the appropriate amine, however purification achieved using column chromatography on the Biotage® QUAD 4 system on a 25S column eluting in 25% EtOAc/i-hex (+AcOH).

LC/MS t=3.89 min [MH⁺] 550/552.

### Example 206

### 3-{2-[5-Bromo-2-(2,4,6-trifluoro-benzyloxy)-phenyl]-5-methyl-pyrrol-1-yl}-5-acetylamino-benzoic acid

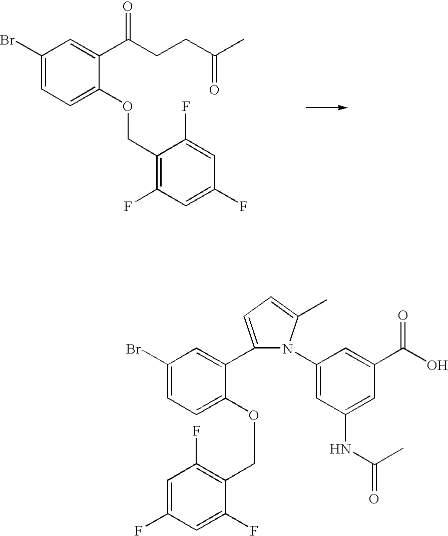

Procedure as for 3-{2-[5-bromo-2-(benzyloxy)-phenyl]-5-methyl-pyrrol-1-yl}-6-chloro-benzoic acid using the appropriate amine, however purification achieved using column chromatography on the Biotage® QUAD 4 system on a 25S column eluting in 40% EtOAc/i-hex (+AcOH).

¹H-NMR (400 MHz, MeOD) 2.11 (6H, s), 4.76 (2H, s), 6.0 (1H, d, J=3 Hz), 6.12 (1H, d, J=3 Hz), 6.79-6.88 (3H, m), 7.24-7.31 (3H, m), 7.49 (1H, s), 8.07 (1H, s).

LC/MS t=3.53 min [MH⁺] 573/575.

### Example 207

### 3-{2-[5-Bromo-2-(2,4,6-trifluoro-benzyloxy)-phenyl]-5-methyl-pyrrol-1-yl}-6-methyl-benzoic acid

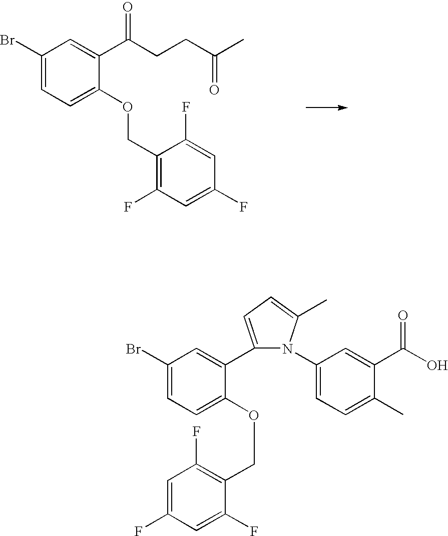

Procedure as for 3-{2-[5-bromo-2-(benzyloxy)-phenyl]-5-methyl-pyrrol-1-yl}-6-chloro-benzoic acid using the appropriate amine, however purification achieved using column chromatography on the Biotage® QUAD 4 system on a 25S column eluting in 25% EtOAc/i-hex (+AcOH).

¹H-NMR (400 MHz, CDCl₃) 2.13 (3H, s), 2.64 (3H, s), 4.76 (2H, s), 6.07 (1H, d, J=3 Hz), 6.23 (1H, d, J=3 Hz), 6.61-6.70 (2H, m), 6.74 (1H, d, J=9 Hz), 7.00 (1H, dd, J=2 Hz, 8 Hz), 7.13 (1H, d, J=9 Hz), 7.23-7.29 (2H+ CDCl₃, m), 7.76 (1H, d, J=2 Hz).

LC/MS t=3.81 min [MH⁺] 530/532.

### Example 208

### 3-{2-[5-Bromo-2-(4-fluoro-benzyloxy)-phenyl]-5-methyl-pyrrol-1-yl}-6-chloro-benzoic acid

### a) S-Bromo-2-(4-fluoro-benzyloxy)-benzaldehyde

Procedure as for 2-benzyloxy-5-chloro-benzaldehyde using the appropriate benzyl bromide to give the title compound.

LCMS t=3.60 min.

### b) 1-[5-Bromo-2-(4-fluoro-benzyloxy)-phenyl]-pentane-1,4-dione

Procedure as for 1-[5-chloro-2-(benzyloxy)-phenyl]-pentane-1,4-dione to give the title compound.

LC/MS t=3.57 min

### c) 3-{2-[5-Bromo-2-(4-fluoro-benzyloxy)-phenyl]-5-methyl-pyrrol-1-yl}-6-chloro-benzoic acid

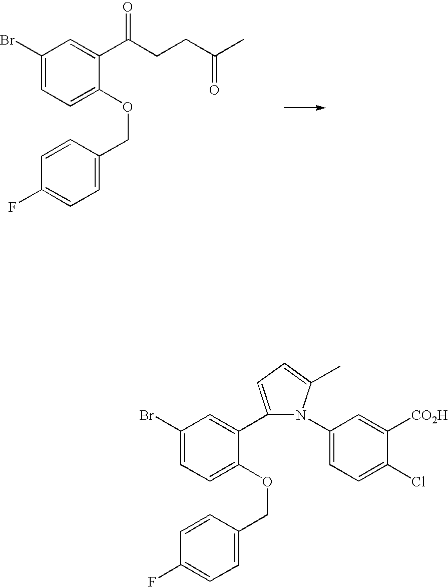

1-[5-Bromo-2-(4-fluoro-benzyloxy)-phenyl]-pentane-1,4-dione (0.1 5g, 0.40 mmol), 5-amino-2-chloro-benzoic acid (0.068g, 0.40 mmol) and p-TSA (cat.) in NMP (2 ml) were heated in a sealed vessel at 180° C. for 15 minutes using microwaves. Upon cooling the reaction was diluted with CH₂Cl₂ (25 ml) and shaken with dil. HCl (1 ml). The organics were separated using a phase separator column with a NaSO₄ cartridge attached and concentrated. The residue was purified by column chromatography using a Si SPE cartridge (5g) eluting in 0-50% EtOAc/i-hex. to give the title compound (1 33 mg, 65%).

¹H-NMR (400 MHz, CDCl₃) 2.15 (3H, s), 4.68 (2H, s), 6.13 (1H, d, J=3 Hz), 6.27 (1H, d, J=3 Hz), 6.54 (1H, d, J=9 Hz), 6.94-7.05 (5H, m), 7.22-7.31 (2H, m), 7.45 (1H, d, J=2.5 Hz), 7.63 (1H, d, J=2.5 Hz).

LC/MS t=4.24 min [MH⁺] 514/516/518.

### Example 209

### 3-{2-[5-Bromo-2-(4-fluoro-benzyloxy)-phenyl]-5-methyl-pyrrol-1-yl}-6-fluoro-benzoic acid

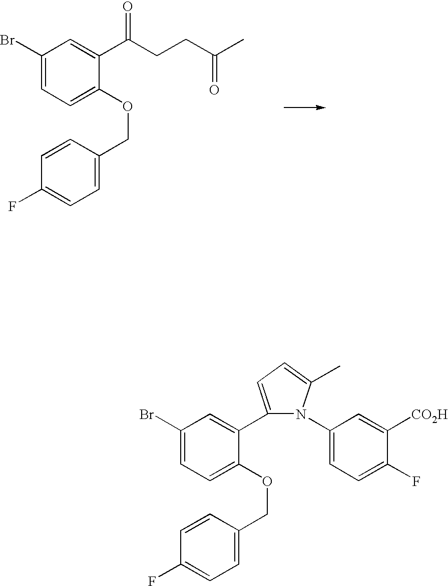

1-[5-Bromo-2-(4-fluoro-benzyloxy)-phenyl]-pentane-1,4-dione (0.109, 0.26 mmol), 5-amino-2-fluoro-benzoic acid (0.041 g, 0.26 mmol) and p-TSA (cat.) in NMP (1 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by MDAP to give the title compound (60 mg, 46%).

¹H-NMR (400 MHz, d6-DMSO) 2.06 (3H, s), 4.84 (2H, s), 6.06 (1H, dd, J=1, 3 Hz), 6.22 (1H, d, J=3 Hz), 6.83 (1H, d, J=9 Hz), 7.12-7.22 (4H, m), 7.24-7.35 (4H, m), 7.41-7.45 (1H, m), 13.40 (1H, s).

LC/MS t=4.06 min [MH⁺] 498/500.

### Example 210

### 3-{2-[5-Bromo-2-(4-fluoro-benzyloxy)-phenyl]-5-methyl-pyrrol-1-yl}-6-methyl-benzoic acid

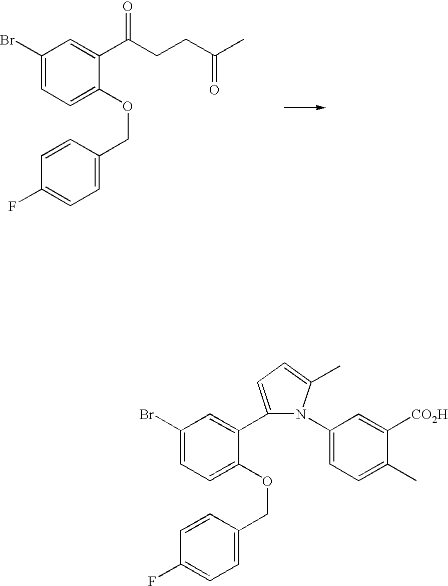

1-[5-Bromo-2-(4-Fluoro-benzyloxy)-phenyl]-pentane-1,4-dione (0.10g, 0.26 mmol), 5-amino-2-methyl-benzoic acid (0.04g, 0.26 mmol) and p-TSA (cat.) in NMP (1 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by MDAP to give the title compound (44 mg, 34%).

¹H-NMR (400 MHz, d6-DMSO) 2.05 (3H, s), 2.50 (excess, s), 4.84 (2H, s), 6.04 (1H, dd, J=1, 3 Hz), 6.20 (1H, d, J=3 Hz), 6.81 (1H, d, J=9 Hz), 7.08-7.19 (5H, m), 7.21-7.32 (3H, m), 7.45 (1H, d, J=2 Hz), 12.95 (1H, s).

LC/MS t=4.05 min [MH⁺] 494/496.

### Example 211

### 3-}2-[5-Bromo-2-(4-fluoro-benzyloxy)-phenyl]-5-methyl-pyrrol-1-yl}-6-hydroxy-benzoic acid

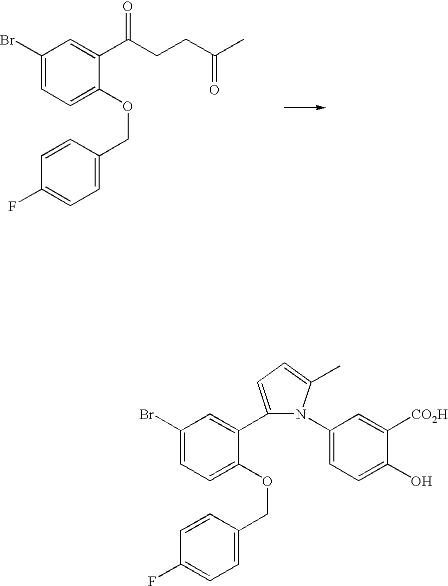

1-[5-Bromo-2-(4-fluoro-benzyloxy)-phenyl]-pentane-1,4-dione (0.30g, 0.78 mmol), 5-amino-2-hydroxy-benzoic acid (0.120g, 0.78 mmol) and p-TSA (cat.) in NMP (3 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by MDAP to give the title compound (78 mg, 20%).

¹H-NMR (400 MHz, CDCl₃) 2.12 (3H, s), 4.74 (2H, s), 6.11 (1H, dd, J=1, 3 Hz), 6.27 (1H, d, J=3 Hz), 6.54 (1H, d, J=9 Hz), 6.82 (1H, d, J=9 Hz), 6.96-7.02 (2H, m), 7.03-7.08 (3H, m), 7.21 (1H, dd, J=3, 9 Hz), 7.39 (1H, d, J=3 Hz), 7.53 (1H, d, J=3 Hz), 10.60 (1H, s).

LC/MS t=4.87 min [MH⁻] 494/496.

### Example 212

### 3-{2-[5-Bromo-2-(4-fluoro-benzyloxy)-phenyl]-5-methyl-pyrrol-1-yl}-5-acetylamino-benzoic acid

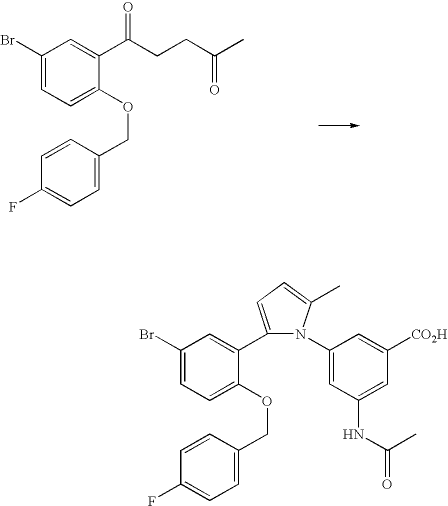

1-[5-Bromo-2-(4-fluoro-benzyloxy)-phenyl]-pentane-1,4-dione (0.30g, 0.78 mmol), 3-acetylamino-5-amino-benzoic acid (0.153g, 0.78 mmol) and p-TSA (cat.) in NMP (3 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by MDAP to give the title compound (89 mg, 21%).

¹H-NMR (400 MHz, d6-DMSO) 2.03 (3H, s), 2.07 (3H, s), 4.83 (2H, s), 6.06 (1H, dd, J=1, 3 Hz), 6.23 (1H, d, J=3 Hz), 6.82 (1.H, d, J=9 Hz), 7.09-7.21 (6H, m), 7.30 (1H, dd, J=3, 9 Hz), 7.59 (1H, d, J=3 Hz), 8.09 (1H, m), 10.10 (1H, s), 13.10 (1H, s).

LC/MS t=3.69 min [MH⁻] 537/539.

### Example 213

### 3-{2-[5-Bromo-2-(4-fluoro-benzyloxy)-phenyl]-5-methyl-pyrrol-1-yl}-1-napthoic acid

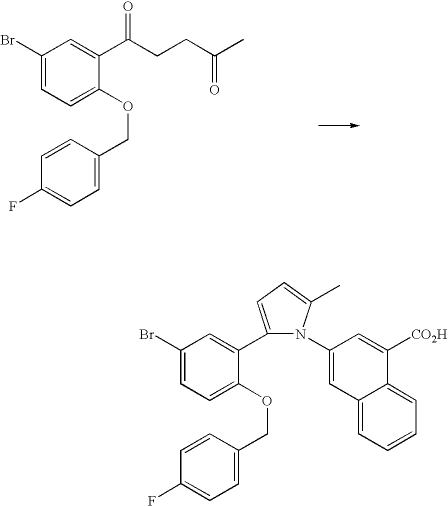

1-[5-Bromo-2-(4-fluoro-benzyloxy)-phenyl]-pentane-1,4-dione (0.10g, 0.26 mmol), 3-amino-napthalene-1-carboxylic acid (0.049g, 0.78 mmol) and p-TSA (cat.) in NMP (1 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by MDAP to give the title compound (79 mg, 56%).

¹H-NMR (400 MHz, d6-DMSO) 2.12 (3H, s), 4.76 (2H, s), 6.12 (1H, dd, J=1, 3 Hz), 6.27 (1H, d, J=3 Hz), 6.73 (1H, d, J=9 Hz), 6.99-7.08 (4H, m), 7.27 (1H, dd, J=3, 9 Hz), 7.34 (1H, d, J=3 Hz), 7.56-7.69 (2H, m), 7.76 (1H, d, J=3 Hz), 7.86-7.91 (2H, m), 8.86 (1H, d, J=9 Hz), 13.25 (1H, s).

LC/MS t=4.21 min [MH⁺] 530/532.

### Example 214

### 3-{2-[5-Bromo-2-(4-fluoro-benzyloxy)-phenyl]-5-methyl-pyrrol-1-yl}-4-fluoro-benzoic acid

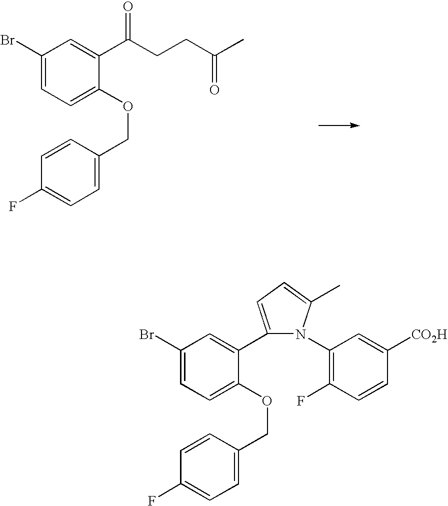

1-[5-Bromo-2-(4-fluoro-benzyloxy)-phenyl]-pentane-1,4-dione (0.10g, 0.26 mmol), 3-amino-4-fluoro-benzoic acid (0.041g, 0.26 mmol) and p-TSA (cat.) in NMP (1 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by MDAP to give the title compound (36 mg, 27%).

¹H-NMR (400 MHz, d6-DMSO) 2.01 (3H, s), 4.85 (2H, dd, J=12 Hz), 6.09 (1H, d, J=3 Hz), 6.27 (1H, d, J=3 Hz), 6.83 (1H, d, J=9 Hz), 7.11-7.24 (5H, m), 7.31 (1H, dd, J=3, 9 Hz), 7.40 (1H, t, J=9 Hz), 7.60 (1H, dd, J=2, 8 Hz), 7.92-7.97 (1H, m), 13.25 (1H, s).

LC/MS t=4.01 min [MH⁻] 496/498.

### Example 216

### 3-{2-[5-Bromo-2-(4-fluoro-benzyloxy)-phenyl]-5-methyl-pyrrol-1-yl}-6-acetylamino-benzoic acid

### a) 3-{2-[5-Bromo-2-(4-fluoro-benzyloxy)-phenyl]-5-methyl-pyrrol-1-yl}-6-acetylamino-benzoic acid methyl ester

1-[5-Bromo-2-(4-fluoro-benzyloxy)-phenyl]-pentane-1,4-dione (0.10g, 0.26 mmol), 2-acetylamino-5-amino-benzoic acid methyl ester (0.055g, 0.26 mmol) and p-TSA (cat.) in NMP (1 ml) were heated in a sealed vessel at 180° C. for 10 minutes using microwaves. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (96 mg, 66%).

LC/MS t=3.85 min [MH⁺] 551/553.

### b) 3-{2-[5-Bromo-2-(4-fluoro-benzyloxy)-phenyl]-5-methyl-pyrrol-1-yl}-6-acetylamino-benzoic acid

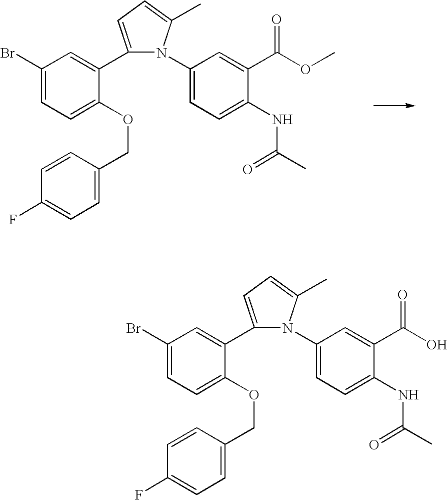

3-{2-[5-Bromo-2-(4-fluoro-benzyloxy)-phenyl]-5-methyl-pyrrol-1-yl}-6-acetylamino-benzoic acid methyl ester (0.095g, 0.17 mmol) was dissolved in methanol (6 ml) and 2M NaOH (0.6 ml) and was heated in a sealed vessel at 100° C. for 60 seconds in a microwave. Upon cooling the reaction was concentrated in vacuo, diluted with CH₂Cl₂ (5 ml) and shaken with dil. HCl (1 ml). The organics were separated using a phase separator column with a Na₂SO₄ cartridge attached and concentrated to give the title compound (93 mg, 100%).

¹H-NMR (400 MHz, CDCl₃) 2.01 (3H, s), 2.20 (3H, s), 4.68 (2H, s), 6.11 (1H, d, J=3 Hz), 6.27 (1H, d, J=3 Hz), 6.53 (1H, d, J=9 Hz), 6.93-7.00 (2H, m), 7.01-7.07 (2H, m), 7.12 (1H, dd, J=3, 9 Hz), 7.19 (1H, dd, J=3, 9 Hz), 7.40 (1H, d, J=3 Hz), 7.69 (1H, d, J=3 Hz), 8.55 (1H, m), 10.95 (1H, s).

LC/MS t=4.30 min [MH⁺] 537/539.

### Example 216

### 3-{2-[5-Bromo-2-(4-fluoro-benzyloxy)-phenyl]-5-methyl-pyrrol-1-yl}-6-difluoromethoxy-benzoic acid

### a) 3-{2-[5-Bromo-2-(4-fluoro-benzyloxy)-phenyl]-5-methyl-pyrrol-1-yl}-6-difluoromethoxy-benzoic acid methyl ester

1-[5-Bromo-2-(4-fluoro-benzyloxy)-phenyl]-pentane-1,4-dione (0.10g, 0.26 mmol), 5-amino-2difluoromethoxy-benzoic acid methyl ester (0.057g, 0.26 mmol) and p-TSA (cat.) in NMP (1 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (45 mg, 30%).

LC/MS t=4.21 min [MH⁺] 560/562.

### b) 3-{2-[5-Bromo-2-(4-fluoro-benzyloxy)-phenyl]-5-methyl-pyrrol-1-yl}-6-difluoromethoxy-benzoic acid

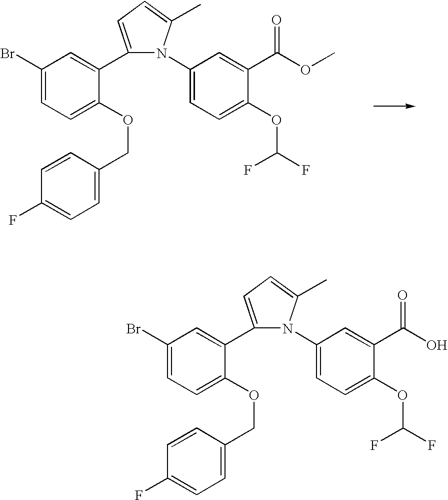

3-{2-[5-Bromo-2-(4-fluoro-benzyloxy)-phenyl]-5-methyl-pyrrol-1-yl}-6-difluoromethoxy-benzoic acid methyl ester (0.045g, 0.08 mmol) was dissolved in methanol (6 ml) and 2M NaOH (0.6 ml) and was heated in a sealed vessel at 100° C. for 60 seconds using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (42 mg, 96%).

¹H-NMR (400 MHz, CDCl₃) 2.13 (3H, s), 4.65 (2H, s), 6.12 (1H, d, J=3 Hz), 6.29 (1H, d, J=3 Hz), 6.53 (1H, d, J=9 Hz), 6.55 (1H, t, J=74 Hz), 6.93-6.99 (2H, m), 7.01-7.08 (4H, m), 7.21 (1H, dd, J=3, 9 Hz), 7.39 (1H, d, J=3 Hz), 7.61 (1H, d, J=2 Hz).

LC/MS t=4.13 min [MH⁺] 546/548.

### Example 217

### 3-{2-[5-Bromo-2-(4-fluoro-benzyloxy)-phenyl]-5-methyl-pyrrol-1-yl}-5-trifluoromethyl-benzoic acid

### a) 3-{2-[5-Bromo-2-(4-fluoro-benzyloxy)-phenyl]-5-methyl-pyrrol-1-yl}-5-trifluoromethyl-benzoic acid methyl ester

1-[5-Bromo-2-(4-Fluoro-benzyloxy)-phenyl]-pentane-1,4-dione (0.10g, 0.26 mmol), 3-amino-5-trifluoromethyl-benzoic acid methyl ester (0.055g, 0.26 mmol) and p-TSA (cat.) in NMP (1 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (50 mg, 34%).

LC/MS t=4.39 min [MH⁺] 562/564.

### b) 3-{2-[5-Bromo-2-(4-fluoro-benzyloxy)-phenyl]-5-methyl-pyrrol-1-yl}-5-trifluoromethyl-benzoic acid

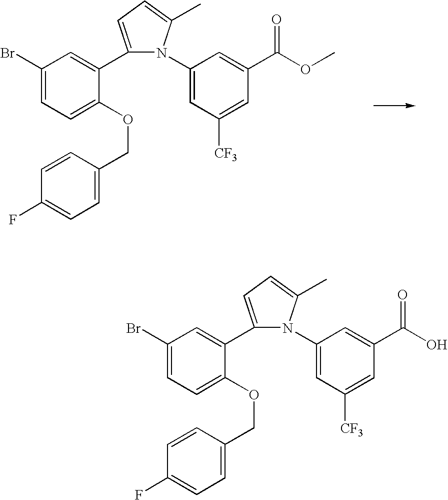

3-{2-[5-Bromo-2-(4-fluoro-benzyloxy)-phenyl]-5-methyl-pyrrol-1-yl}-5-trifluoromethyl-benzoic acid methyl ester (0.050g, 0.09 mmol) was dissolved in methanol (6 ml) and 2M NaOH (0.6 ml) and was heated in a sealed vessel at 100° C. for 60 seconds using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (38 mg, 80%).

¹H-NMR (400 MHz, CDCl₃) 2.18 (3H, s), 4.64 (2H, s), 6.17 (1H, dd, J=0.5, 3 Hz), 6.31 (1H, d, J=3 Hz), 6.53 (1H, d, J=9 Hz), 6.93-7.04 (4H, m), 7.24 (1H, dd, J=3, 9 Hz), 7.40 (1H, m), 7.45 (1H, d, J=2.5 Hz), 7.85 (1H, s), 8.18 (1H, m).

LC/MS t=4.39 min [MH⁺] 548/550.

### Example 218

### 3-{2-[5-Bromo-2-(4-fluoro-benzyloxy)-phenyl]-5-methyl-pyrrol-1-yl}-5-amino-benzoic acid

### a) 3-{2-[5-Bromo-2-(4-fluoro-benzyloxy)-phenyl]-5-methyl-pyrrol-1-yl}-5-amino-benzoic acid methyl ester

1-[5-Bromo-2-(4-fluoro-benzyloxy)-phenyl]-pentane-1,4-dione (1.0g, 2.6 mmol), 3,5-diaminobenzoic acid methyl ester (0.44g, 2.6 mmol) and p-TSA (cat.) in NMP (5 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (10g) eluting in 1-20% EtOAc/i-hex. to give the title compound (750 mg, 56%).

LC/MS t=3.99 min [MH⁺] 509/511.

### b) 3-{2-[5-Bromo-2-(4-fluoro-benzyloxy)-phenyl]-5-methyl-pyrrol-1-yl}-5-amino-benzoic acid

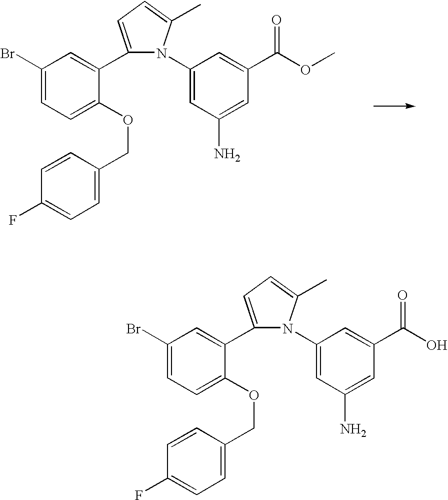

3-{2-[5-Bromo-2-(4-fluoro-benzyloxy)-phenyl]-5-methyl-pyrrol-1-yl}-5-amino-benzoic acid methyl ester (0.15g, 0.23 mmol) was dissolved in methanol (6 ml) and 2M NaOH (0.6 ml) and was heated in a sealed vessel at 120° C. for 5 minutes using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (11 0 mg, 97%).

¹H-NMR (400 MHz, CDCl₃) 2.15 (3H, s), 4.74 (2H, s), 6.10 (1H, dd, J=0.5, 3 Hz), 6.28 (1H, d, J=3 Hz), 6.43 (1H, t, J=2 Hz), 6.55 (1H, d, J=9 Hz), 6.93-7.01 (2H, m), 7.05-7.11 (2H, m), 7.16 (1H, t, J=1.5 Hz), 7.19 (1H, dd, J=3, 9 Hz), 7.24-7.27 (1H, m), 7.36 (1H, d, J=2.5 Hz).

LC/MS t=3.79 min [MH⁺] 1495/497.

### Example 219

### 3-{2-[5-Bromo-2-(4-fluoro-benzyloxy)-phenyl]-5-ethyl-pyrrol-1-yl}-5-(1,1-dioxo-1I⁶-isothiazolidin-2-yl)-benzoic acid

### a) 3-{2-[5-Bromo-2-(4-fluoro-benzyloxy)-phenyl]-5-methyl-pyrrol-1-yl}-5-(1,1-dioxo-1I⁶-isothiazolidin-2-yl)-benzoic acid methyl ester

1-[5-Bromo-2-(4-fluoro-benzyloxy)-phenyl]-pentane-1,4-dione (0.10 g, 0.26 mmol), 3-amino-5-(1,1-dioxo-1I⁶-isothiazolidin-2-yl)-benzoic acid methyl ester (0.071g, 0.26 mmol) and P TSA (cat.) in NMP (1 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (108 mg, 67%).

LC/MS t=3.98 min [MH⁺] 613/615.

### b) 3-{2-[5-Bromo-2-(4-fluoro-benzyloxy)-phenyl]-5-methyl-pyrrol-1-yl}-5-(1,1-dioxo-1I⁶-isothiazolidin-2-yl)-benzoic acid

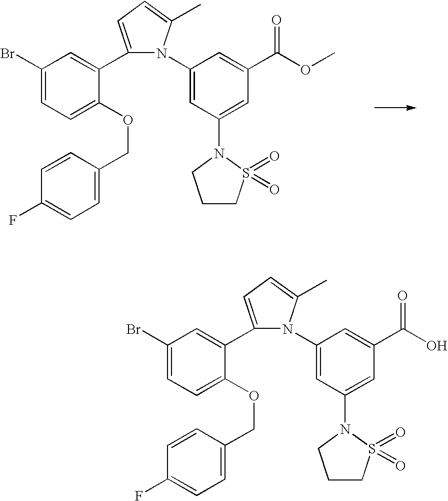

3-{2-[5-Bromo-2-(4-fluoro-benzyloxy)-phenyl]-5-methyl-pyrrol-1-yl}-5-(1,1-dioxo-1I⁶-isothiazolidin-2-yl)-benzoic acid methyl ester (0.108g) was dissolved in methanol (6 ml) and 2M NaOH (0.6 ml) and was heated in a sealed vessel at 100° C. for 60 seconds using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (100 mg, 95%).

¹H-NMR (400 MHz, CDCl₃) 2.20 (3H, s), 2.48 (2H, quin, J=9 Hz), 3.34 (2H, t, J=8.5 Hz), 3.50 (2H, t, J=8.5 Hz), 4.71 (2H, s), 6.13 (1H, d, J=3 Hz), 6.28 (1H, d, J=3 Hz), 6.53 (1H, d, J=9 Hz), 6.92-6.99 (2H, m), 7.00-7.05 (2H, m), 7.11 (1H, t, J=2 Hz), 7.21 (1H, dd, J=3, 9 Hz), 7.42 (1H, d, J=2.5 Hz), 7.48 (1H, t, J=2 Hz), 7.76 (1H, t, J=2 Hz).

LC/MS t=3.84 min [MH⁺] 599/601.

### Example 220

### 3-{2-[5-Bromo-2-(4-fluoro-benzyloxy)-phenyl]-5-methyl-pyrrol-1-yl}-5-(2-oxo-pyrrolidin-1-yl)-benzoic acid

### a) 3-{2-[5-Bromo-2-(4-fluoro-benzyloxy)-phenyl]-5-methyl-pyrrol-1-yl}-(2-oxo-pyrrolidin-1-yl)-benzoic acid methyl ester

1-[5-Bromo-2-(4-fluoro-benzyloxy)-phenyl]-pentane-1,4-dione (0.10g, 0.26 mmol), 3-amino-5-(2-oxo-pyrrolidin-1-yl)-benzoic acid methyl ester (0.062g, 0.26 mmol) and p-TSA (cat.) in NMP (1 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (94 mg, 67%).

LC/MS t=4.02 min [MH⁺] ] 577/579.

### b) 3-{2-[6-Bromo 2-(4-fluoro-benzyloxy)-phenyl]-5-methyl-pyrrol-1-yl}-5-(2-oxo-pyrrolidin-1-yl)-benzoic acid

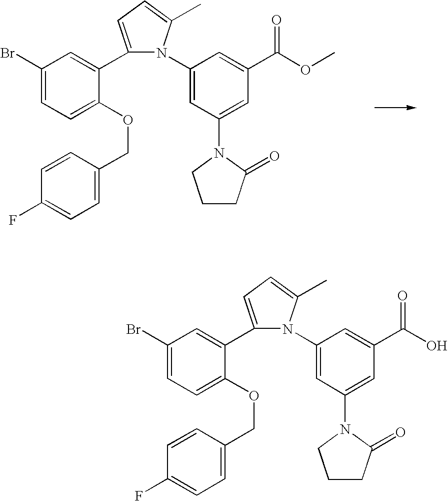

3-{2-[5-Bromo-2-(4-fluoro-benzyloxy)-phenyl]-5-methyl-pyrrol-1-yl}-5-(2-oxo-pyrrolidin-1-yl)-benzoic acid methyl ester (0.094g, 0.16 mmol) was dissolved in methanol (6 ml) and 2M NaOH (0.6 ml) and was heated in a sealed vessel at 100° C. for 60 seconds using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (90 mg, 100%).

¹H-NMR (400 MHz, CDCl₃) 2.10 (2H, quin, J=8 Hz), 2.19 (3H, s), 2.57 (2H, t, J=8 Hz), 3.58 (2H, t, J=7.5 Hz), 4.71 (2H, s), 6.13 (1H, d, J=3 Hz), 6.29 (1H, d, J=3 Hz), 6.52 (1H, d, J=9 Hz), 6.92-6.98 (2H, m), 7.00-7.06 (2H, m), 7.19 (1H, dd, J=3, 9 Hz), 7.40 (1H, d, J=2.5 Hz), 7.49 (1H, t, J=2 Hz), 7.57 (1H, t, J=2 Hz), 8.17 (1H, t, J=1.5 Hz).

LC/MS t=3.89 min [MH⁺] 563/565.

### Example 221

### 3-{2-[5-Bromo-2-(4-fluoro-benzyloxy)-phenyl]-5-methyl-pyrrol-1-yl}-5-(2-oxo-piperidin-1-yl)-benzoic acid

### a) 3-{2-[5-Bromo-2-(4-fluoro-benzyloxy)-phenyl]-5-methyl-pyrrol-1-yl}-(2-oxo-piperidin-1-yl)-benzoic acid methyl ester

1-[5-Bromo-2-(4-fluoro-benzyloxy)-phenyl]-pentane-1,4-dione (0.10 g, 0.26 mmol), 3-amino-5-(2-oxo-piperidin-1-yl)-benzoic acid methyl ester (0.065g, 0.26 mmol) and p-TSA (cat.) in NMP (1 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (47 mg, 30%).

LC/MS t=3.96 min [MH⁺] 591/593.

### b) 3-{2-[5-Bromo-2-(4-fluoro-benzyloxy)-phenyl]-5-methyl-pyrrol-1-yl}-5-(2-oxo-piperidin-1-yl)-benzoic acid

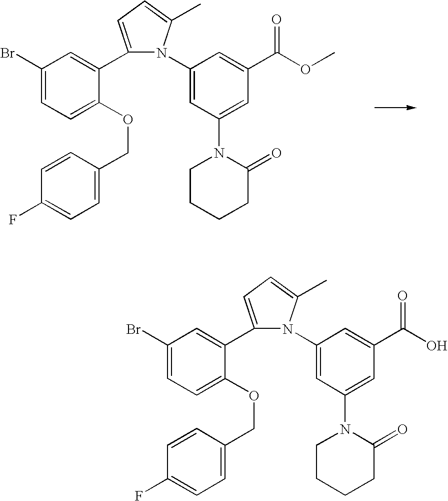

3-{2-[5-Bromo-2-(4-fluoro-benzyloxy)-phenyl]-5-methyl-pyrrol-1-yl}-5-(2-oxo-piperidin-1-yl)-benzoic acid methyl ester (0.047g, 0.08 mmol) was dissolved in methanol (6 ml) and 2M NaOH (0.6 ml) and was heated in a sealed vessel at 100° C. for 60 seconds using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (45 mg, 100%).

¹H-NMR (400 MHz, CDCl₃) 1.60-1.67 (4H, m), 2.02 (3H, s), 2.27-2.32 (2H, m), 3.02-3.09 (2H, m), 4.60 (2H, s), 6.02 (1H, d, J=3 Hz), 6.25 (1H, d, J=3 Hz), 6.43 (1H, d, J=9 Hz), 6.71 (1H, s), 6.86-6.97 (2H, m), 6.99-7.05 (3H, m), 7.24 (1H, d, J=3 Hz), 7.58 (1H, s), 7.84 (1H, s).

LC/MS t=3.96 min [MH⁺] 591/593.

### Example 222

### 3-{2-[5-Bromo-2-(4-fluoro-benzyloxy)-phenyl]-5-methyl-pyrrol-1-yl}-5-amino-6-methyl-benzoic acid

### a) 3-{2-[5-Bromo-2-(4-fluoro-benzyloxy)-phenyl]-5-methyl-pyrrol-1-yl}-5-amino-6-methyl-benzoic acid methyl ester

1-[5-Bromo-2-(4-fluoro-benzyloxy)-phenyl]-pentane-1,4-dione (0.10g, 0.26 mmol), 3,5-diamino-2-methyl-benzoic acid methyl ester (0.047g, 0.26 mmol) and p-TSA (cat.) in NMP (1 ml) were heated in a sealed vessel at 180° C. for 10 minutes using a microwave. Upon cooling the reaction was diluted with EtOAc (25 ml) and washed with dil. NaHCO₃, dil. HCl and brine, dried (MgSO₄), filtered and concentrated. The residue was purified by column chromatography using a Si SPE cartridge (5g) eluting in 1-20% EtOAc/i-hex. to give the title compound (70 mg, 52%).

LC/MS t=4.02 min [MH⁺] 523/525.

### b) 3-{2-[5-Bromo-2-(4-fluoro-benzyloxy)-phenyl]-5-methyl-pyrrol-1-yl}-5-amino-6-methyl-benzoic acid

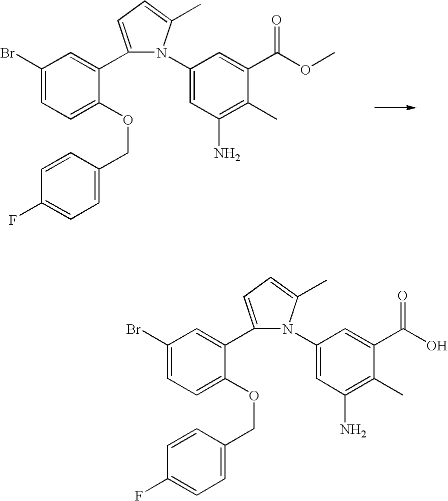

3-{2-[5-Bromo-2-(4-fluoro-benzyloxy)-phenyl]-5-methyl-pyrrol-1-yl}-5-amino-2-methyl-benzoic acid methyl ester (0.070g, 0.13 mmol) was dissolved in methanol (6 ml) and 2M NaOH (0.6 ml) and was heated in a sealed vessel at 120° C. for 5 minutes using a microwave. Upon cooling the reaction was concentrated in vacuo, diluted with CH₂Cl₂ (5 ml) and shaken with dil. HCl (1 ml). The organics were separated using a phase separator column with a NaSO₄ cartridge attached and concentrated to give the title compound (66 mg, 97%).

¹H-NMR (400 MHz, CDCl₃) 1.83 (3H, s), 1.87 (3H, s), 3.21 (2H, broad s), 4.69 (2H, s), 5.93 (1H, d, J=3 Hz), 6.12 (1H, d, J=2 Hz), 6.24 (1H, d, J=3 Hz), 6.39 (1H, d, J=9 Hz), 6.63 (1H, d, J=2 Hz), 6.83-6.91 (3H, m), 7.02-7.08 (2H, m), 7.13 (1H, d, J=2.5 Hz).

LC/MS t=3.81 min [MH⁺] 509/511.

### Example 223

### 3-{2-[5-Fluoro-2-(4-fluoro-benzyloxy)-phenyl]-5-methyl-pyrrol-1-yl}-6-chloro-benzoic acid

### a) 5-Fluoro-2-(4-fluoro-benzyloxy)-benzaldehyde

5-Fluorosalicylaldehyde (4.8g, 34.3 mmol), 4-fluorobenzyl bromide (4.32 ml, 34.3 mmol) and K₂CO₃ (9.5g, 68.6 mmol) were heated in DMF (50 ml) at 50° C. for 30mins. Upon cooling to room temperature, EtOAc and sat. NH₄Cl were added. The layers were separated and the aqueous phase was extracted with EtOAc (×2). The combined organic extracts were washed with water, dried (MgSO₄), filtered and concentrated to give the title compound (8.4g, 98%).

¹H NMR (400 MHz, CDCl₃) 5.14 (2H, s), 7.01 (1H, dd, J=4, 9 Hz), 7.07-7.14 (2H, m), 7.22-7.28 (1H, m), 7.37-7.44 (2H, m), 7.53 (1H, dd, J=3.5, 8 Hz), 10.45 (1H, s).

LC/MS t=3.45 min.

### b) 1-[5-Fluoro-2-(4-fluoro-benzyloxy)-phenyl]-pentane-1,4-dione

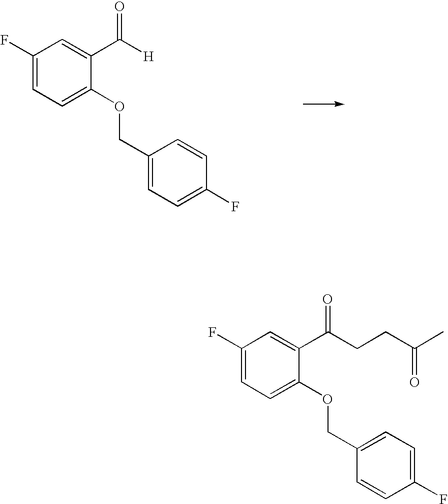

A mixture of 5-Fluoro-2-(4-fluoro-benzyloxy)-benzaldehyde (6.4g, 25.8 mmol), methyl vinyl ketone (2.19 ml, 26.3 mmol), 3-ethyl-5-(2-hydroxyethyl)-4-methylthiazolium bromide (1.95g, 7.7 mmol) and triethylamine (10.7 ml, 77 mmol) was heated in ethanol (5 ml) at 80° C. for 16 hours. Upon cooling, the mixture was diluted with EtOAc (100 ml) and washed with saturated NH₄Cl, brine, dried (Na₂SO₄) filtered and concentrated. The residue was purified by chromatography using Biotage with iso-hexane containing a gradient of EtOAc (5-20%) to give the title compound as an oil (5.68g, 69%).

¹H NMR (400 MHz, CDCl₃) 2.19 (3H, s), 2.78 (2H, t, J=6 Hz), 3.22 (2H, t, J=6 Hz), 5.10 (2H, s), 6.96 (1H, dd, J=4, 9 Hz), 7.05-7.17 (3H, m), 7.38-7.48 (3H, m).

LC/MS t=3.17 min

### c) 3-{2-[5-Fluoro-2-(4-fluoro-benzyloxy)-phenyl]-5-methyl-pyrrol-1-yl}-6-chloro-benzoic acid

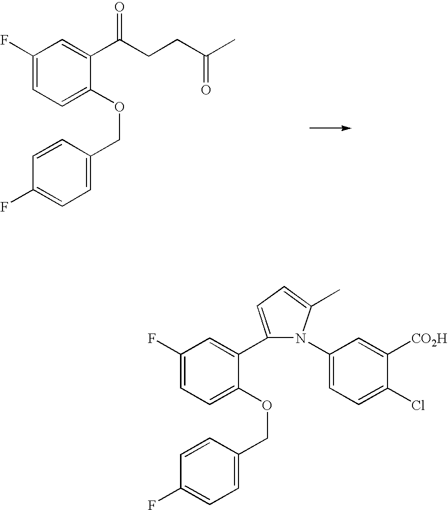

1-[5-Fluoro-2-(4-fluoro-benzyloxy)-phenyl]-pentane-1,4-dione (0.1 5g, 0.47 mmol), 5amino-2-chloro-benzoic acid (0.081 g, 0.47 mmol) and p-TSA (cat.) in CH₃CN (1 ml) were heated in a sealed vessel at 1 60° C. for 10 minutes using a microwave. Upon cooling the reaction was diluted with CH₂Cl₂ (5 ml) and shaken with dil. HCl (1 ml). The organics were separated using a phase separator column with a Na₂SO₄ cartridge attached and concentrated. The residue was purified by column chromatography using Biotage reverse phase 25M cartridge, with water/CH₃CN (30-100%) as eluant, to give the title compound (94 mg, 44%).

¹H-NMR (400 MHz, CDCl₃) 2.15 (3H, s), 4.67 (2H, s), 6.14 (1H, d, J=3 Hz), 6.30 (1H, d, J=3 Hz), 6.62 (1H, dd, J=3, 9 Hz), 6.84 (1H, m), 6.94-7.07 (6H, m), 7.25-7.30 (1H excess, m), 7.60 (1H, d, J=2.5 Hz).

LC/MS t=3.79 [MH⁺] 454/456.

### Example 224

### 3-{2-[5-Fluoro-2-(4-fluoro-benzyloxy)-phenyl]-5-methyl-pyrrol-1-yl}-6-fluoro-benzoic acid

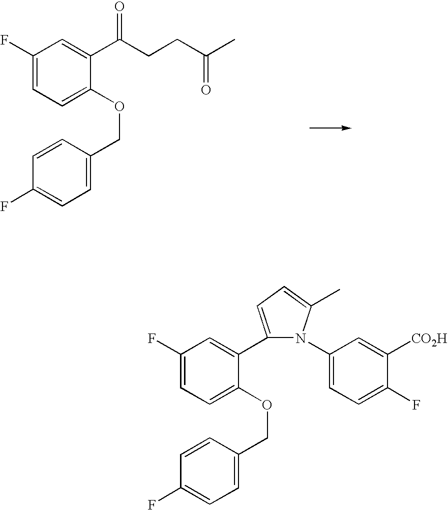

1-[5-Fluoro-2-(4-fluoro-benzyloxy)-phenyl]-pentane-1,4-dione (0.15g, 0.47 mmol), 5-amino-2-fluoro-benzoic acid (0.073g, 0.47 mmol) and p-TSA (cat.) in CH₃CN (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a Na₂SO₄ cartridge attached and concentrated. The residue was purified by column chromatography using Biotage reverse phase 25M cartridge, with water/CH₃CN (30-100%) as eluant, to give the title compound (142 mg, 69%).

¹H-NMR (400 MHz, d6-DMSO) 2.14 (3H, s), 4.70 (2H, s), 6.13 (1H, d, J=3 Hz), 6.30 (1H, d, J=3 Hz), 6.63 (1H, dd, J=4, 9 Hz), 6.80-6.87 (1H, m), 6.94-7.03 (4H, m), 7.05-7.12 (3H, m), 7.63 (1H, dd, J=2.5, 7 Hz).

LC/MS t=3.88 min [MH⁺] 438.

### Example 225

### 3-{2-[5-Fluoro-2-(4-fluoro-benzyloxy)-phenyl]-5-methyl-pyrrol-1-yl}-6-methyl-benzoic acid

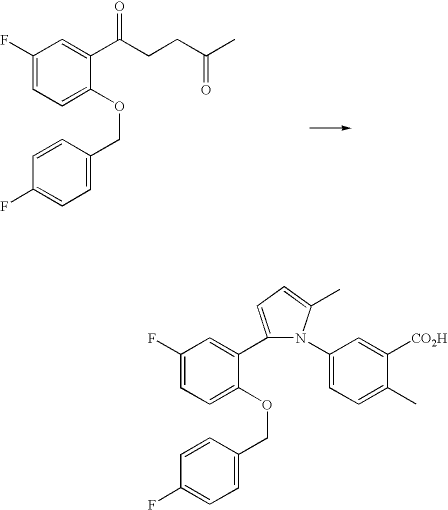

1-[5-Fluoro-2-(4-fluoro-benzyloxy)-phenyl]-pentane-1,4-dione (0.15g, 0.47 mmol), 5-amino-2-methyl-benzoic acid (0.071g, 0.47 mmol) and p-TSA (cat.) in CH₃CN (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage reverse phase 25M cartridge, with water/CH₃CN (30-100%) as eluant, to give the title compound (142 mg, 69%).

¹H-NMR (400 MHz, CDCl₃) 2.15 (3H, s), 2.61 (3H, s), 4.71 (2H, s), 6.12 (1H, d, J=3 Hz), 6.31 (1H, d, J=3 Hz), 6.60 (1H, dd, J=4, 9 Hz), 6.76-6.82 (1H, m), 6.91-7.02 (4H, m), 7.04-7.12 (3H, m), 7.71 (1H, d, J=2 Hz).

LC/MS t=3.89 min [MH⁺] 434.

### Example 226

### 3-(2-[5-Fluoro-2-(4-fluoro-benzyloxy)-phenyl]-5-methyl-pyrrol-1-yl}-6-hydroxy-benzoic acid

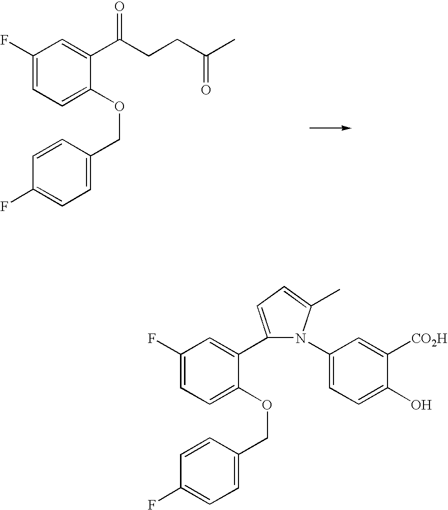

1-[5-Fluoro-2-(4-fluoro-benzyloxy)-phenyl]-pentane-1,4-dione (0.30g, 0.94 mmol), 5-amino-2-hydroxy-benzoic acid (0.144g, 0.94 mmol) and p-TSA (cat.) in CH₃CN (2 ml) were heated in a sealed vessel at 160° C. for 10 minutes using microwaves. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage reverse phase 25M cartridge, with water/CH₃CN (30-100%) as eluant, to give the title compound (256 mg, 62%).

¹H-NMR (400 MHz, CDCl₃) 2.13 (3H, s), 4.73 (2H, s), 6.12 (1H, d, J=3.5 Hz), 6.30 (1H, d, J=3.5 Hz), 6.63 (1H, dd, J=4.5, 9 Hz), 6.78-6.85 (2H, m), 6.92-7.03 (3H, m), 7.05-7.12 (3H, m), 7.52 (1H, d, J=2.5 Hz), 10.40 (1H, s).

LC/MS t=4.47 min [MH⁺] 436.

### Example 227

### 3-{2-[5-Fluoro-2-(4-fluoro-benzyloxy)-phenyl]-5-methyl-pyrrol-1-yl}-5-acetylamino-benzoic acid

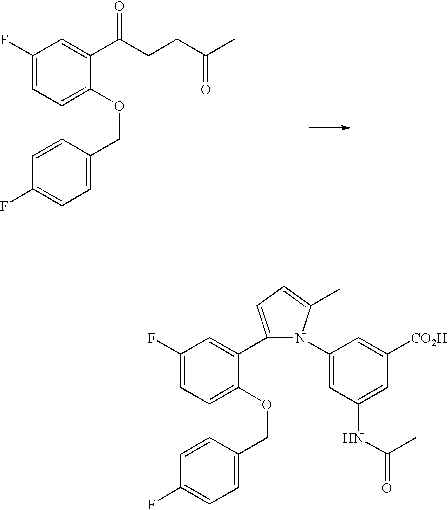

1-[5-Fluoro-2-(4-fluoro-benzyloxy)-phenyl]-pentane-1,4-dione (0.30g, 0.94 mmol), 3-acetylamino-5-amino-benzoic acid (0.183g, 0.94 mmol) and p-TSA (cat.) in CH₃CN (2 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage reverse phase 25M cartridge, with water/CH₃CN (30-100%) as eluant, to give the title compound (347 mg, 77%).

¹H-NMR (400 MHz, CDCl₃) 2.01 (3H, s), 2.08 (3H, s), 4.70 (2H, s), 6.12 (1H, d, J=3 Hz), 6.32 (1H, d, J=3 Hz), 6.61 (1H, dd, J=4.5, 9 Hz), 6.76-6.82 (1H, m), 6.91-7.02 (3H, m), 7.06-7.14 (3H, m), 7.42 (1H, s), 7.51 (1H, s), 7.96 (1H, s).

LC/MS t=3.56 min [MH⁺] 477.

### Example 228

### 3-{2-[5-Fluoro-2-(4-fluoro-benzyloxy)-phenyl]-5-methyl-pyrrol-1-yl}-napthalene-1-carboxylic acid

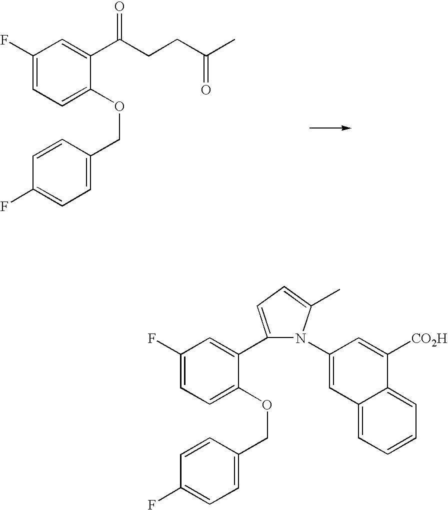

1-[5-Fluoro-2-(4-fluoro-benzyloxy)-phenyl]-pentane-1,4-dione (0.15g, 0.47 mmol), 3-amino-napthalene-1-carboxylic acid (0.088g, 0.47 mmol) and p-TSA (cat.) in CH₃CN (1 ml) were heated in a sealed vessel at 160° C. for 10 minutes using microwaves. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage reverse phase 25M cartridge, with water/CH₃CN (30-100%) as eluant, to give the title compound (52 mg, 24%).

¹H-NMR (400 MHz, CDCl₃) 2.21 (3H, s), 4.60 (2H, s), 6.19 (1H, d, J=3 Hz), 6.36 (1H, d, J=3 Hz), 6.50 (1H, dd, J=4.5, 9 Hz), 6.73-6.80 (1H, m), 6.86-6.97 (4H, m), 7.09 (1H, dd, J=3, 9 Hz), 7.48-7.68 (4H, m), 8.05 (1H, d, J=2 Hz), 9.03 (1H, d, J=8.5 Hz).

LC/MS t=4.04 min [MH⁺] 470.

### Example 229

### 3-{2-[5-Fluoro-2-(4-fluoro-benzyloxy)-phenyl]-5-methyl-pyrrol-1-yl}-4-fluoro-benzoic acid

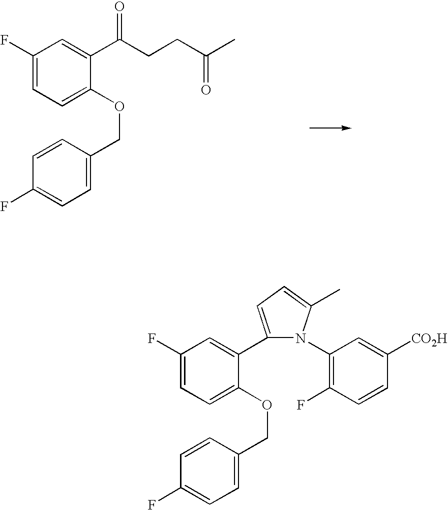

1-[5-Fluoro-2-(4-fluoro-benzyloxy)-phenyl]-pentane-1,4-dione (0.15g, 0.47 mmol), 3-amino-4-fluoro-benzoic acid (0.073g, 0.47 mmol) and p-TSA (cat.) in CH₃CN (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage reverse phase 25M cartridge, with water/CH₃CN (30-100%) as eluant, to give the title compound (94 mg, 46%).

¹H-NMR (400 MHz, CDCl₃) 2.11 (3H, s), 4.76 (2H, s), 6.16 (1H, d, J=3 Hz), 6.34 (1H, d, J=3 Hz), 6.58 (1H, dd, J=4, 9 Hz), 6.74-6.80 (1H, m), 6.90-7.01 (3H, m), 7.05-7.13 (3H, m), 7.77 (1H, dd, J=2, 7 Hz), 7.98-8.04 (1H,m).

LC/MS t=3.85 min [MH⁺] 438.

### Example 230

### 3-{2-[5-Fluoro-2-(4-fluoro-benzyloxy)-phenyl]-5-methyl-pyrrol-1-yl}-6-difluoromethoxy-benzoic acid

### a) 3-{2-[5-Fluoro-2-(4-fluoro-benzyloxy)-phenyl]-5-methyl-pyrrol-1-yl}-6-difluoromethoxy-benzoic acid methyl ester

1-[5-Fluoro-2-(4-fluoro-benzyloxy)-phenyl]-pentane-1,4-dione (0.15g, 0.47 mmol), 5-amino-2-difluoromethoxy-benzoic acid methyl ester (0.102g, 0.47 mmol) and p-TSA (cat.) in CH₃CN (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a Na₂SO₄ cartridge attached and concentrated. The residue was purified by column chromatography using Biotage 25M cartridge, with iso-hexane/EtOAc (5-50%) as eluant, to give the title compound (130 mg, 55%).

LC/MS t=3.98 min [MH⁺] 500.

### b) 3-{2-[5-Fluoro-2-(4-fluoro-benzyloxy)-phenyl]-5-methyl-pyrrol-1-yl}-6-difluoromethoxy-benzoic acid

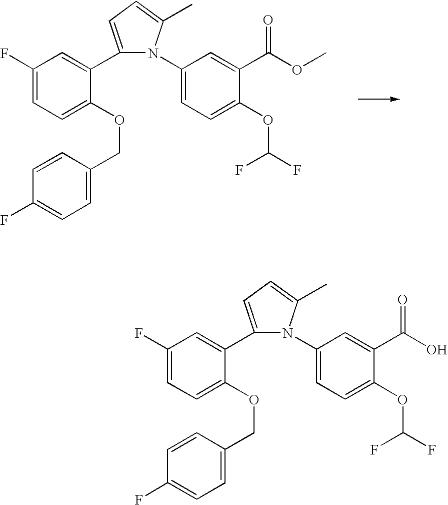

3-{2-[5-Fluoro-2-(4-fluoro-benzyloxy)-phenyl]-5-methyl-pyrrol-1-yl}-6-difluoromethoxy-benzoic acid methyl ester (0.130g, 0.26 mmol) was dissolved in ethanol (2 ml) and 2M NaOH (0.5 ml) and was heated in a sealed vessel at 120° C. for 5 minutes using microwaves. Upon cooling the reaction was diluted with CH₂Cl₂ (8 ml) and shaken with dil. HCl (1 ml). The organics were separated using a phase separator column with a Na₂SO₄ cartridge attached and concentrated to give the title compound (100 mg, 79%).

¹HNMR (400 MHz, CDCl₃) 2.16 (3H, s), 4.65 (2H, s), 6.14 (1H, d, J=3.5 Hz), 6.30 (1H, d, J=3.5 Hz), 6.59 (1H, t, J=74 Hz), 6.62 (1H, dd, J=4.5, 9 Hz), 6.80-6.87 (1H, m), 6.95-7.03 (3H, m), 7.04-7.11 (4H, m), 7.62-7.64 (1H, m).

LC/MS t=3.67 min [MH⁺] 486.

### Example 231

### 3-{2-[5-Fluoro-2-(4-fluoro-benzyloxy)-phenyl]-5-methyl-pyrrol-1-yl}-5-trifluoromethyl-benzoic acid

### a) 3-{2-[5-Fluoro-2-(4-fluoro-benzyloxy)-phenyl]-5-methyl-pyrrol-1-yl}-5-trifluoromethyl-benzoic acid methyl ester

1-[5-Fluoro-2-(4-fluoro-benzyloxy)-phenyl]-pentane-1,4-dione (0.15g, 0.47 mmol), 3-amino-5-trifluoromethyl-benzoic acid methyl ester (0.099g, 0.47 mmol) and p-TSA (cat.) in CH₃CN, (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage 25M cartridge, with iso-hexane/EtOAc (5-50%) as eluant, to give the title compound (110 mg, 47%).

LC/MS t=4.16 min [MH⁺] 502.

### b) 3-{2-[5-Fluoro-2-(4-fluoro-benzyloxy)-phenyl]-5-methyl-pyrrol-1-yl}-5-trifluoromethyl-benzoic acid

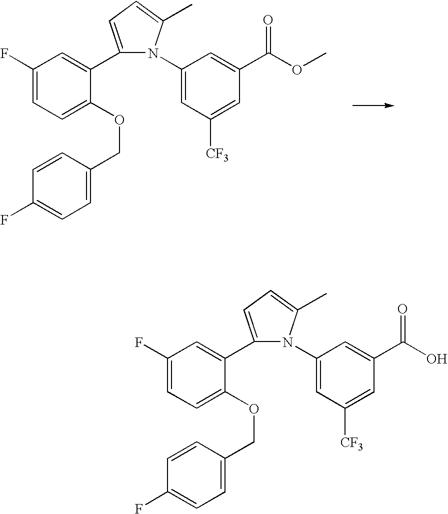

3-{2-[5-Fluoro-2-(4-fluoro-benzyloxy)-phenyl]-5-methyl-pyrrol-1-yl}-5-trifluoromethyl-benzoic acid methyl ester (0.110 g, 0.22 mmol) was dissolved in ethanol (2 ml) and 2M NaOH (0.5 ml) and was heated in a sealed vessel at 120° C. for 5 minutes using a microwave. Upon cooling the reaction was diluted with CH₂Cl₂ (8 ml) and shaken with dil. HCl (1 ml). The organics were separated using a phase separator column with a Na₂SO₄ cartridge attached and concentrated to give the title compound (97 mg, 90%).

¹H NMR (400 MHz, CDCl₃) 2.18 (3H, s), 4.61 (2H, s), 6.18 (1H, d, J=3 Hz), 6.32 (1H, d, J=3 Hz), 6.60 (1H, dd, J=4.5, 9 Hz), 6.82-6.88 (1H, m), 6.94-7.06 (5H, m), 7.37 (1H, s), 7.83 (1H, s), 8.17 (1H, s).

LC/MS t=3.86 min [MH⁺] 488.

### Example 232

### 3-{2-[5-Fluoro-2-(4-fluoro-benzyloxy)-phenyl]-5-methyl-pyrrol-1-yl}-5-(1,1-dioxo-1I⁶-isothiazolidin-2-yl)-benzoic acid

### a) 3-{2-[5-Fluoro-2-(4-fluoro-benzyloxy)-phenyl]-5-methyl-pyrrol-1-yl}-5-1,1-dioxo-1I⁶-isothiazolidin-2-yl)-benzoic acid methyl ester

1-[5-Fluoro-2-(4-fluoro-benzyloxy)-phenyl]-pentane-1,4-dione (0.15g, 0.47 mmol), 3-amino-5-(1,1-dioxo-1I⁶-isothiazolidin-2-yl)-benzoic acid methyl ester (0.127g, 0.47 mmol) and p-TSA (cat.) in CH₃CN (1 ml) were heated in a sealed vessel at 160° C. for 10 minutes using microwaves. Upon cooling the reaction was diluted with CH₂Cl₂ (5 ml) and shaken with dil. HCl (1 ml). The organics were separated using a phase separator column with a Na₂SO₄ cartridge attached and concentrated. The residue was purified by column chromatography using Biotage 25M cartridge, with iso-hexane/EtOAc (1 580%) as eluant, to give the title compound (160 mg, 61%).

LC/MS t=3.79 min [MH⁺] 553.

### b) 3-{2-[5-Fluoro-2-(4-fluoro-benzyloxy)-phenyl]-5-methyl-pyrrol-1-yl}-5-(1,1-dioxo-1I⁶-isothiazolidin-2-yl)-benzoic acid

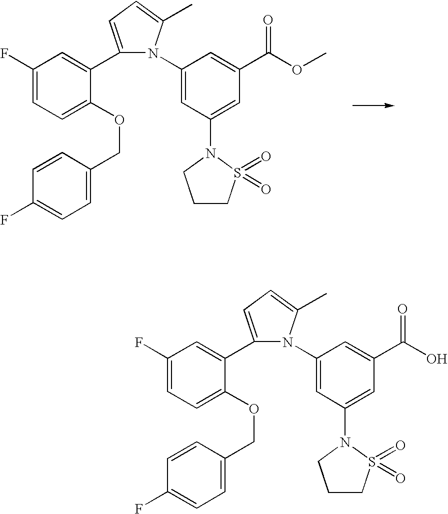

3-{2-[5-Fluoro-2-(4-fluoro-benzyloxy)-phenyl]-5-methyl-pyrrol-1-yl}-5-(1,1-dioxo-1I⁶-isothiazolidin-2-yl)-benzoic acid methyl ester (0.160g, 0.29 mmol) was dissolved in ethanol (2 ml) and 2M NaOH (0.5 ml) and was heated in a sealed vessel at 120° C. for 5 minutes using a microwave. Upon cooling the reaction was diluted with CH₂Cl₂ (8 ml) and shaken with dil. HCl (1 ml). The organics were separated using a phase separator column with a Na₂SO₄ cartridge attached and concentrated to give the title compound (1 55 mg, 100%).

¹H-NMR (400 MHz, CDCl₃) 2.21 (3H, s), 2.49 (2H, quin, J=7 Hz), 3.34 (2H, t, J=7 Hz), 3.52 (2H, t, J=7 Hz), 4.70 (2H, s), 6.14 (1H, d, J=3 Hz), 6.32 (1H, d, J=3 Hz), 6.61 (1H, dd, J=4.5, 9 Hz), 6.78-6.95 (1H, m), 6.94-7.01 (3H, m), 7.03-7.09 (2H, m), 7.13-7.17 (1H, m), 7.45-7.47 (1H, m), 7.72-7.75 (1H, m).

LC/MS t=3.43 min [MH⁺] 539.

### Example 233

### 3-{2-[5-Fluoro-2-(4-fluoro-benzyloxy)-phenyl]-5-methyl-pyrrol-1-yl}-5-(2-oxo-pyrrolidin-1-yl)-benzoic acid

### a) 3-{2-[5-Fluoro-2-(4-fluoro-benzyloxy)-phenyl]-5-methyl-pyrrol-1-yl}-5-(2-oxo-pyrrolidin-1-yl)-benzoic acid methyl ester

1-[5-Fluoro-2-(4-fluoro-benzyloxy)-phenyl]-pentane-1,4-dione (0.15g, 0.47 mmol), 3-amino-5-{2-oxo-pyrrolidin-1-yl)-benzoic acid methyl ester (0.110 g, 0.47 mmol) and p-TSA (cat.) in CH₃CN (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage 25M cartridge, with iso-hexane/EtOAc (15-80%) as eluant, to give the title compound (135 mg, 55%).

LC/MS t=3.80 min [MH⁺] 517.

### b) 3-{2-[5-Fluoro-2-(4-fluoro-benzyloxy)-phenyl-56methyl-pyrrol-1-yl}-5-(2-oxo-pyrrolidin-1-yl)-benzoic acid

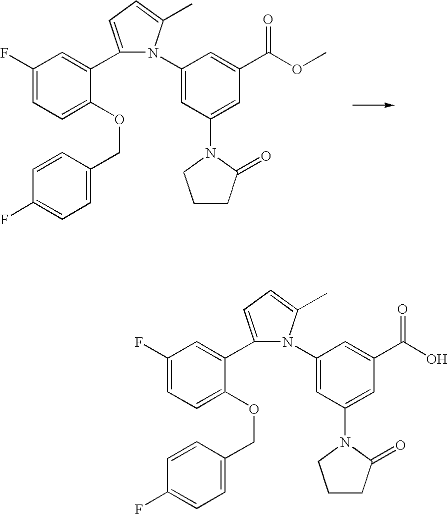

3-{2-[5-Fluoro-2-(4-fluoro-benzyloxy)-phenyl]-5-methyl-pyrrol-1-yl}-5-(2-oxo-pyrrolidin-1-yl)-benzoic acid methyl ester (0.135g, 0.26 mmol) was dissolved in ethanol (2 ml) and 2M NaOH (0.5 ml) and was heated in a sealed vessel at 120° C. for 5 minutes using microwaves. Upon cooling the reaction was diluted with CH₂Cl₂ (8 ml) and shaken with dil. HCl (1 ml). The organics were separated using a phase separator column with a NaSO₄ cartridge attached and concentrated to give the title compound (98 mg, 75%).

¹H-NMR (400 MHz, d6-DMSO) 2.01 (2H, quin, J=7 Hz), 2.10 (3H, s), 2.45-2.53 (2H excess, m), 3.61-3.67 (2H, m), 4.77 (2H, s), 6.08 (1H, d, J=3 Hz), 6.25 (1H, d, J=3 Hz), 6.86 (1H, dd, J=4.5, 9 Hz), 6.91-7.03 (2H, m), 7.10-7.20 (4H, m), 7.23 (1H, s), 7.56-7.59 (1H, m), 8.16 (1H, s).

LC/MS t=3.65 min [MH⁺] 503.

### Example 234

### 3-{2-[5-Fluoro-2-(4-fluoro-benzyloxy)-phenyl]-5-methyl-pyrrol-1-yl}-5-amino-6-methyl-benzoic acid

### a) 3-{2-[5-Fluoro-2-(4-fluoro-benzyloxy)-phenyl]-5-methyl-pyrrol-1-yl}-5-amino-6-methyl-benzoic acid methyl ester

1-[5-Fluoro-2-(4-fluoro-benzyloxy)-phenyl]-pentane-1,4-dione (0.1 5g, 0.47 mmol), 3,5-diamino-2-methyl-benzoic acid methyl ester (0.085g, 0.47 mmol) and p-TSA (cat.) in CH₃CN (1 ml) were heated in a sealed vessel at 160° C. for 10 minutes using a microwave. Upon cooling the reaction was diluted with CH₂Cl₂ (5 ml) and shaken with dil. HCl (1 ml). The organics were separated using a phase separator column with a NaSO₄ cartridge attached and concentrated. The residue was purified by column chromatography using Biotage 25M cartridge, with iso-hexane/EtOAc (7-50%) as eluant, to give the title compound (125 mg, 57%).

LC/MS t=3.81 min [MH⁺] 463.

### b) 3-{2-[5-Fluoro-2-(4-fluoro-benzyloxy)-phenyl]-5-methyl-pyrrol-1-yl}-5-amino-6-methyl-benzoic acid

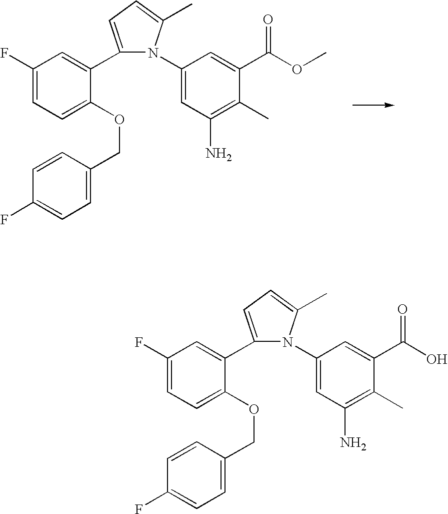

3-{2-[5-Fluoro-2-(4-fluoro-benzyloxy)-phenyl]-5-methyl-pyrrol-1-yl}-5-amino-6-methyl-benzoic acid methyl ester (0.125g, 0.27 mmol) was dissolved in ethanol (2 ml) and 2M NaOH (0.5 ml) and was heated in a sealed vessel at 120° C. for 5 minutes using a microwave. Upon cooling the reaction was diluted with CH₂Cl₂ (8 ml) and shaken with dil. HCl (1 ml). The organics were separated using a phase separator column with a NaSO₄ cartridge attached and concentrated to give the title compound (115 mg, 95%).

¹H-NMR (400 MHz, d6-DMSO) 2.05 (3H, s), 2.20 (3H, s), 4.90 (2H, s), 6.00 (1H, d, J=3.5 Hz), 6.23 (1H, d, J=3.5 Hz), 6.58 (1H, s), 6.69 (1H, s), 6.78 (1H, dd, J=3, 9 Hz), 6.86-6.98 (2H, m), 7.12-7.19 (2H, m), 7.23-7.29 (2H, m).

LC/MS t=3.60 min [MH⁺] 449.

### Example 235

### 3-{2-[5-Fluoro-2-(2,4-difluoro-benzyloxy)-phenyl]-5-methyl-pyrrol-1-yl}-6-methyl-benzoic acid

### a) 5-Fluoro-2-2,4-difluoro-benzyloxy)-benzaldehyde

5-Fluorosalicylaldehyde (5.0g, 35.7 mmol), 2,4-difluorobenzyl bromide (4.62 ml, 35.7 mmol) and K₂CO₃ (4.9g, 35.7 mmol) were heated in DMF (50 ml) at 50° C. for 30mins. Upon cooling to room temperature, EtOAc and sat. NH₄Cl were added. The layers were separated and the aqueous phase was extracted with EtOAc (×2). The combined organic extracts were washed with water, dried (MgSO₄), filtered and concentrated to give the title compound (9.3g, 98%).

¹H NMR (400 MHz, CDCl₃) 5.19 (2H, s), 6.85-6.97 (2H, m), 7.06 (1H, dd, J=4, 9 Hz), 7.23-7.30 (1H excess, m), 7.42-7.50 (2H, m), 7.53 (1H, dd, J=3.5, 8 Hz), 10.45 (1H, s).

LC/MS t=3.48 min.

### b) 1-[5-Fluoro-2-(2,4-difluoro-benzyloxy)-phenyl]-pentane-1,4-dione

A mixture of 5-Fluoro-2-(2,4-difluoro-benzyloxy)-benzaldehyde (9.3g, 35 mmol), methyl vinyl ketone (2.92 ml, 35 mmol), 3-ethyl-5-(2-hydroxyethyl)-4-methylthiazolium bromide (2.48g, 9.8 mmol) and triethylamine (14.5 ml, 105 mmol) was heated in ethanol (8 ml) at 80° C. for 16 hours. Upon cooling, the mixture was diluted with EtOAc (100 ml) and washed with saturated NH₄Cl, brine, dried (Na₂SO₄) filtered and concentrated. The residue was purified by chromatography using Biotage with iso-hexane containing a gradient of EtOAc (5-20%) to give the title compound as an oil (7.36g, 63%).

LC/MS t=3.40 min [MNa⁺] 359.

### c) 3-{2-[5-Fluoro-2-(2,4-difluoro-benzyloxy)-phenyl]-5-methyl-pyrrol-1-yl}-6-methyl-benzoic acid

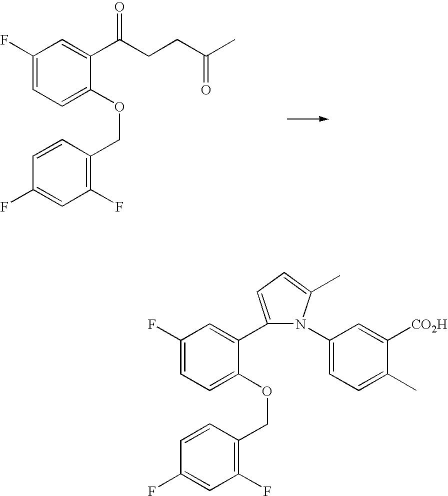

1-[5-Fluoro-2-(2,4-difluoro-benzyloxy)-phenyl]-pentane-1,4-dione (0.1 5g, 0.44 mmol), 5-amino-2-methyl-benzoic acid (0.067g, 0.44 mmol) and p-TSA (cat.) in CH₃CN (1 ml) were heated in a sealed vessel at 160° C. for 10 minutes using microwaves. Upon cooling the reaction was diluted with CH₂Cl₂ (5 ml) and shaken with dil. HCl (1 ml). The organics were separated using a phase separator column with a Na₂SO₄ cartridge attached and concentrated. The residue was purified by chromatography, with iso-hexane/EtOAc (10-30%) as eluant, to give the title compound (82 mg, 41%).

¹H-NMR (400 MHz, CDCl₃) 2.16 (3H, s), 2.62 (3H, s), 4.76 (2H, s), 6.12 (1H, d, J=3 Hz), 6.31 (1H, d, J=3 Hz), 6.65 (1H, dd, J=4, 9 Hz), 6.73-6.85 (3H, m), 6.92 (1H, dd, J=3, 9 Hz), 6.99-7.14 (3H, m), 7.74 (1H, d, J=2 Hz).

LC/MS t=4.01 min [MH⁺] 452.

### Example 236

### 6-{2-[5-Trifluoromethyl-2-(benzyloxy)-phenyl]-5-methyl-pyrrol-1-yl}-picolinic acid

### a) 6-{2-[5-Trifluoromethyl-2-(benzyloxy)-phenyl]-5-methyl-pyrrol-1-yl}-2-bromo-pyridine

1-[5-trifluoromethyl-2-(benzyloxy)-phenyl]-pentane-1,4dione (1.5g, 4.3 mmol), 2-amino-6-bromopyridine (0.75g, 4.3 mmol) and p-TSA (10 mg, cat.) in CH₃CN (5 ml) were heated in a sealed vessel at 200° C. for 1.5 hours using microwaves. Upon cooling the reaction was concentrated and the residue was purified by chromatography on silica gel with iso-hexane/EtOAc (5%) as eluant, to give the title compound (645 mg, 31%).

LC/MS t=4.14 min [MH⁺] 487/489.

### b) 6-{2-[5-Trifluoromethyl-2-(benzyloxy)-phenyl]-5-methyl-pyrrol-1-yl}-picolinic acid

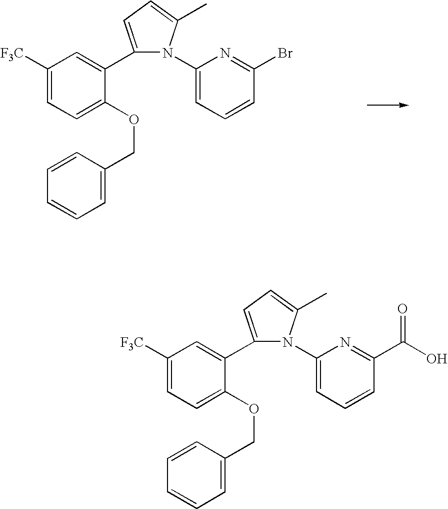

n-Butyl lithium (1.6M in hexanes, 0.94 ml, 1.5 mmol) was added to 6-[2-(5-trifluoromethyl-2-benzyloxy-phenyl)-5-methyl-pyrrol-1-yl]-2-bromo-pyridine (0.49g, 1 mmol) in THF (1 5 ml) at −78° C. After 50 minutes at this temperature, solid CO₂ was added and the solution warmed to room temperature. The solution was concentrated in vacuo and the residue triturated with iso-hexane/2% EtOAc. The off white solid was filtered, washed with further iso-hexane and air dried to give the title compound (0.38g, 84%).

¹H NMR (400 MHz, d6-DMSO) 2.15 (3H, s), 5.08 (2H, s), 6.05 (1H, d, J=3 Hz), 6.41 (1H, d, J=3 Hz), 6.77 (1H, d, J=8 Hz), 7.04 (1H, d, J=2 Hz), 7.11 (1H, d, J=8 Hz), 7.28-7.43 (6H, m), 7.67 (1H, t, J=8 Hz), 7.81 (1H, d, J=8 Hz).

LC/MS t=3.88 min [MH⁺] 453.

### Example 237

### 6-{2-[5-Trifluoromethyl-2-(4-fluoro-benzyloxy)-phenyl]-5-methyl-pyrrol-1-yl}-picolinic-acid

### a) 6-{2-[5-Trifluoromethyl-2-(hydroxy)-phenyl]-5-methyl-pyrrol-1-yl}-picolinic acid

6-{2-[5-Trifluoromethyl-2-(benzyloxy)-phenyl]-5-methyl-pyrrol-1-yl}-picolinic acid (0.38g, 0.84 mmol), ammonium formate (0.265 mg, 4.2 mmol) and 10% Pd/C (50% wet, 0.080g, ˜0.04 mmol) were heated in ethanol (5 ml) at 60° C. for 1 hour. The mixture was cooled, filtered through Celite®, washing through with EtOAc. The solution was concentrated in vacuo to give the title compound (0.42 g).

LC/MS t=3.64 min [MH⁺] 363.

### b) 6-{2-[5-Trifluoromethyl-2-(4-fluoro-benzyloxy)-phenyl]-5-methyl-pyrrol-1-yl}-picolinic-acid-(4-fluoro-benzyl)-ester

6-{2-[5-Trifluoromethyl-2-(hydroxy)-phenyl]-5-methyl-pyrrol-1-yl}-picolinic acid (0.10 g, 0.28 mmol), 4-fluorobenzyl bromide (0.070 ml, 0.56 mmol) and K₂CO₃ (0.85g, 0.61 mmol) were heated in DMF (1.2 ml) at 60° C. for 16 hours. Further 4-fluorobenzyl bromide (0.070 ml, 0.56 mmol) was added and heating continued for a further 24 hours. The mixture was cooled, diluted with CH₂Cl₂ (5 ml) and shaken with water (1 ml). The organics were separated using a phase separator column with a Na₂SO₄ cartridge attached and concentrated. The residue was purified by chromatography on silica gel, with iso-hexane/EtOAc (10-15%) as eluant, to give the title compound (70 mg, 44%).

LC/MS t=4.19 min [MH⁺] 579.

### c) 6-{2-[5-Trifluoromethyl-2-(4-fluoro-benzyloxy)-phenyl]-5-methyl-pyrrol-1-yl}-picolinic-acid

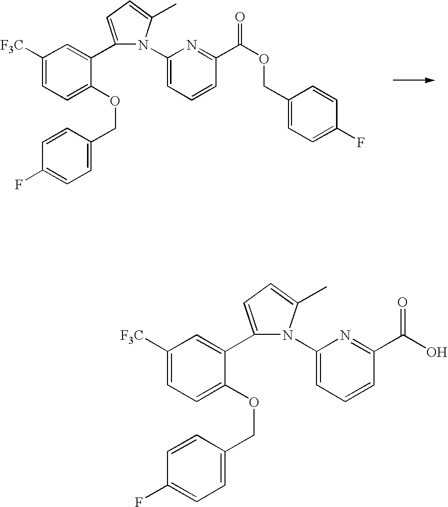

6-{2-[5-Trifluoromethyl-2-(4-fluoro-benzyloxy)-phenyl]-5-methyl-pyrrol-1-yl}-picolinic-acid-(4fluoro-benzyl)-ester (0.07g, 0.1 2 mmol) was dissolved in ethanol (4 ml) and 2M NaOH (1 ml) and was heated in a sealed vessel at 120° C. for 15 minutes using a microwave. Upon cooling the reaction was diluted with CH₂Cl₂ (1 0 ml) and shaken with dil. HCl (1 ml). The organics were separated using a phase separator column with a Na₂SO₄ cartridge attached and concentrated. The residue was triturated with iso-hexane/2% EtOAc. The off white solid was filtered, washed with further iso-hexane and air dried to give the title compound (45 mg).

¹H NMR (400 MHz, CDCl₃) 2.31 (3H, s), 4.61 (2H, s), 6.20 (1H, d, J=3.5 Hz), 6.38 (1H, d, J=3.5 Hz), 6.77 (1H, d, J=8 Hz), 7.01 (4H, d, J=8 Hz), 7.05 (1H, d, J=8 Hz), 7.47 (1H, dd, J=2, 8 Hz), 7.61 (1H, d, J=2 Hz), 7.75 (1H, t, J=8 Hz), 8.01 (1H, d, J=8 Hz).

LC/MS t=3.87 [MH⁺] 471.

### Example 238

### 6-{2-[5-Trifluoromethyl-2-2,3-difluoro-benzyloxy)-phenyl]-5-methyl-pyrrol-1-yl}-picolinic-acid

### a) 6-{2-[5-Trifluoromethyl-2-(2,3-difluoro-benzyloxy)-phenyl]-5-methyl-pyrrol-1-yl}-picolinic-acid-(2,3-difluoro-benzyl)-ester

6-{2-[5-Trifluoromethyl-2-(hydroxy)-phenyl]-5-methyl-pyrrol-1-yl}-picolinic acid (0.10 g, 0.28 mmol), 2,3-difluorobenzyl bromide (0.072 ml, 0.56 mmol) and K₂CO₃ (0.859, 0.61 mmol) were heated in DMF (1.2 ml) at 60° C. for 16 hours. Further 2,3-difluorobenzyl bromide (0.072 ml, 0.56 mmol) was added and heating continued for a further 24 hours. The mixture was cooled, diluted with CH₂Cl₂ (5 ml) and shaken with water (1 ml). The organics were separated using a phase separator column with a Na₂SO₄ cartridge attached and concentrated. The residue was purified by chromatography on silica gel, with iso-hexane/EtOAc (15%) as eluant, to give the title compound (40 mg, 24%).

LC/MS t=4.21 min [MH⁺] 615.

### b) 6-{2-[5-Trifluoromethyl-2-(2,3-difluoro-benzyloxy)-phenyl]-5-methyl-pyrrol-1-yl}-picolinic-acid

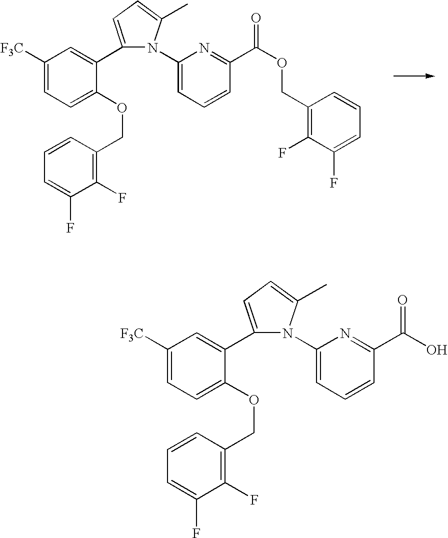

6-{2-[5-Trifluoromethyl-2-(2,3difluoro-benzyloxy)-phenyl]-5-methyl-pyrrol-1-yl}-picolinic-acid-(2,3-difluoro-benzyl)-ester (0.049, 0.07 mmol) was dissolved in ethanol (4 ml) and 2M NaOH (1 ml) and was heated in a sealed vessel at 120° C. for 15 minutes using a microwave. Upon cooling the reaction was diluted with CH₂Cl₂ (1 0 ml) and shaken with dil. HCl (1 ml). The organics were separated using a phase separator column with a NaSO₄ cartridge attached and concentrated. The residue was triturated with iso-hexane/2% EtOAc. The off white solid was filtered, washed with further iso-hexane and air dried to give the title compound (25 mg).

¹H NMR (400 MHz, d6-DMSO) 2.20 (3H, s), 5.04 (2H, s), 6.08 (1H, d, J=3 Hz), 6.35 (1H, d, J=3 Hz), 7.00-7.08 (2H, m), 7.14-7.23 (3H, m), 7.36-7.46 (1H, m), 7.52 (1H, d, J=8 Hz), 7.83 (1H, d, J=8 Hz), 7.90 (1H, d, J=8 Hz).

LC/MS t=3.88 min [MH⁺] 489.

### Example 239

### 6-{2-[5-Trifluoromethyl-2-(2,6-difluoro-benzyloxy)-phenyl]-5-methyl-pyrrol-1-yl}-picolinic-acid

### a) 6-{2-[5-Trifluoromethyl-2-(2,6-difluoro-benzyloxy)-phenyl]-5-methyl-pyrrol-1-yl}-picolinic-acid-(2,6-difluoro-benzyl)-ester

6-{2-[5-Trifluoromethyl-2-(hydroxy)-phenyl]-5-methyl-pyrrol-1-yl}-picolinic acid (0.10g, 0.28 mmol), 2,6-difluorobenzyl bromide (0.15g, 0.56 mmol) and K₂CO₃ (0.85g, 0.61 mmol) were heated in DMF (1.2 ml) at 60° C. for 16 hours. Further 2,6-difluorobenzyl bromide (0.15g, 0.56 mmol) was added and heating continued for a further 24 hours. The mixture was cooled, diluted with CH₂Cl₂ (5 ml) and shaken with water (1 ml). The organics were separated using a phase separator column with a NaSO₄ cartridge attached and concentrated. The residue was purified by chromatography on silica gel, with iso-hexane/EtOAc (15%) as eluant, to give the title compound (75 mg, 44%).

LC/MS t=4.15 min [MH⁺] 615.

### b) 6-{2-[5-Trifluoromethyl-2-(2,6-difluoro-benzyloxy)-phenyl]-5-methyl-pyrrol-1-yl}-picolinic-acid

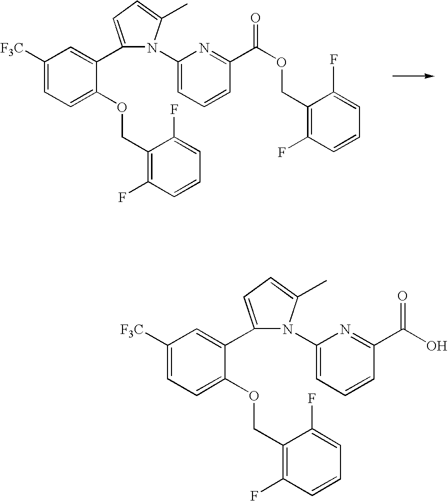

6-{2-[5-Trifluoromethyl-2-(2,6-difluoro-benzyloxy)-phenyl]-5-methyl-pyrrol-1-yl}-picolinic-acid-(2,6-difluoro-benzyl)-ester (0.07g, 0.1 1 mmol) was dissolved in ethanol (4 ml) and 2M NaOH (1 ml) and was heated in a sealed vessel at 120° C. for 15 minutes using a microwave. Upon cooling the reaction was diluted with CH₂Cl₂ (1 0 ml) and shaken with dil. HCl (1 ml). The organics were separated using a phase separator column with a NaSO₄ cartridge attached and concentrated. The residue was triturated with iso-hexane/2% EtOAc. The off white solid was filtered, washed with further iso-hexane and air dried to give the title compound (22 mg).

¹H NMR (400 MHz, CDCl₃) 2.27 (3H, s), 4.79 (2H, s), 6.12 (1H, s), 6.32 (1H, d, J=3 Hz), 6.84-7.06 (4H, m), 7.27-7.38 (1H, m), 7.47-7.55 (2H, m), 7.73-7.79 (1H, m), 8.01 (1H, d, J=7 Hz).

LC/MS t=3.81 min [MH⁺] 489.

### Example 240

### 6-{2-[5-Chloro-2-(4-fluoro-benzyloxy)-phenyl]-5-methyl-pyrrol-1-yl}-picolinic acid

### a) 6-{2-[5-Chloro-2-(4-fluoro-benzyloxy)-phenyl]-5-methyl-pyrrol-1-yl}-2-bromo-pyridine

1-[5-Chloro-2-(4-fluorobenzyloxy)-phenyl]-pentane-1,4-dione (2.05g, 6.1 mmol), 2-amino-4-bromopyridine (1.06g, 6.1 mmol) and p-TSA (10 mg, cat.) in CH₃CN (5 ml) were heated in a sealed vessel at 200° C. for 2 hours using a microwave. Upon cooling the reaction was concentrated and the residue was purified by chromatography on silica gel with iso-hexane/EtOAc (1-10%) as eluant, to give the title compound (550 mg, 19%).

LC/MS t=4.15 min [MH⁺] 471/473/475.

### b) 6-{2-[5-Chloro-2-(4-fluoro-benzyloxy)-phenyl]-5-methyl-pyrrol-1-yl}-picolinic acid

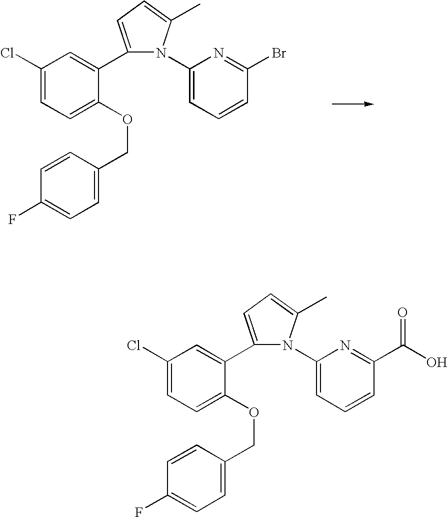

n-Butyl lithium (1.6M in hexanes, 0.94 ml, 1.5 mmol) was added to 6-{2-[5-chloro-2-(4-fluoro-benzyloxy)-phenyl]-5-methyl-pyrrol-1-yl}-2-bromo-pyridine (0.55g, 1.17 mmol) in THF (1 5 ml) at −78° C. After 1 hour at this temperature, solid CO₂ was added and the solution warmed to room temperature. The solution was concentrated in vacuo and the residue was purified by MDAP to give the title compound (60 mg).

¹H NMR (400 MHz, CDCl₃) 2.32 (3H, s), 4.51 (2H, s), 6.18 (1H, d, J=3.5 Hz), 6.33 (1H, d, J=3.5 Hz), 6.62 (1H, d, J=9 Hz), 6.98 (4H, d, J=7 Hz), 7.04 (1H, d, J=8 Hz), 7.16 (1H, dd, J=2.5, 8 Hz), 7.37 (1H, d, J=2.5 Hz), 7.74 (1H, t, J=8 Hz), 8.00 (1H, d, J=8 Hz).

LC/MS t=3.91 min [MH⁺] 437/439.

### Example 241

### 6-{2-[5-Bromo-2-(4-fluoro-benzyloxy)-phenyl]-5-methyl-pyrrol-1-yl}-picolinic acid

### a) 6-{2-[5-Bromo-2-(4-fluoro-benzyloxy)-phenyl]-5-methyl-pyrrol-1-yl}-2-bromo-pyridine

Procedure as for 3-{2-[5-Trifluoromethyl-2-(4-fluoro-benzyloxy)-phenyl]-5-methyl-pyrrol-1yl}-6-chloro-benzoic acid using the appropriate amine.

LC/MS t=4.25 min [MH⁺] 515/517/519.

### b) 6-{2-[5-Bromo-2-(4-fluoro-benzyloxy)-phenyl]-5-methyl-pyrrol-1-yl}-picolinic acid ethyl ester

6-{2-[5-Bromo-2-(4-fluoro-benzyloxy)-phenyl]-5-methyl-pyrrol-1yl}-2-bromo pyridine (0.10g, 0.194 mmol, 1 eq) was dissolved in ethanol (2 ml) and bis(triphenylphosphine)palladium(II)chloride (0.0048 g, 0.0068 mmol, 0.035 eq), and triethylamine (0.6 ml) were added, and the solution saturated with CO_{(g)} for 10 min with stirring. The reaction was heated to 70° C. with stirring under CO_{(g)} for 28 hours, and bis(triphenylphosphine)palladium(II)chloride (0.0048 g, 0.0068 mmol, 0.035 eq), and triethylamine (0.6 ml) were added, the solution was re-saturated with CO_{(g)} for 10 mins and heated to 70° C. for a further 18 hours. The reaction mixture was diluted with EtOAc, and washed with water, the combined organic extracts were washed with brine, dried (MgSO₄), filtered and the volatiles were removed in vacuo. The residue was then purified by chromatography using Biotage Flash 12+S cartridge with 5% EtOAc:iso-hexane as the eluant to yield the title compound (0.056g, 0.1 1 mmol, 57%) as a yellow solid.

LC/MS t=4.10 min [MH+] 509/511.

### c) 6-{2-[5-Bromo-2-(4-fluoro-benzyloxy)-phenyl]-5-methyl-pyrrol-1-yl}-picolinic acid

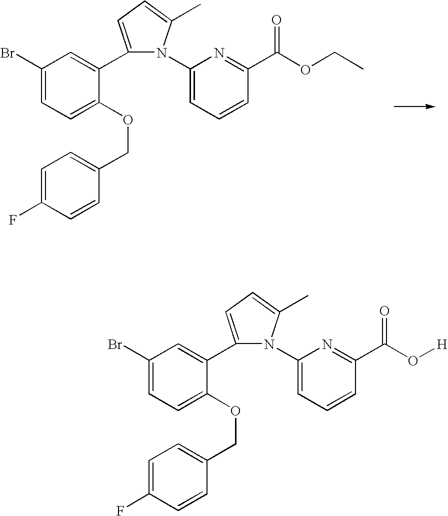

Procedure as for 3-{2-[5-Trifluoromethyl-2-(4-fluoro-benzyloxy)-phenyl]-5-methyl-pyrrol-1 yl}-5-(1,1-dioxo-1I⁶-isothiazolidin-2-yl)-benzoic acid.

¹H NMR (400 MHz, CDCl₃) 2.30 (3H, s), 4.51 (2H, s), 6.17 (1H, d, J=3 Hz), 6.32 (1H, d, J=3 Hz), 6.57 (1H, d, J=8 Hz), 6.98 (4H, d, J=8 Hz), 7.03 (1H, d, J=8 Hz), 7.29 (1H, dd J=8 Hz, 2 Hz), 7.50, (1H, d, J=2 Hz), 7.73 (1H, t, J=8 Hz), 8.00 (1H, d, J=8 Hz).

LC/MS t=4.15 min [MH+] 481/483.

### Example 242

### 3-{2-[5-Trifluoromethyl-2-(4-fluoro-benzyloxy)-phenyl]-5-methyl-pyrrol-1-yl}-6-chloro-benzoic acid

### a) 2-(4-Fluoro-benzyloxy)-5-trifluoromethyl-benzaldehyde

2-Hydroxy-5-trifluoromethyl-benzaldehyde (1 0.00g, 0.053 mol, 1 eq) was added to DMF (100 ml), K₂CO₃ (14.53g, 0.105 mol, 2 eq) and p-fluorobenzylbromide (9.95g, 0.053 mol, 1 eq) were then added to the stirred reaction mixture. The reaction was stirred at room temperature for 1 hour. The reaction mixture was quenched with water and extracted with EtOAc. The combined organic extracts were washed with brine, dried (MgSO₄), filtered and the volatiles were removed in vacuo to yield title compound (13.00 g, 0.044 mol, 82%) as a dark orange oil.

LC/MS t=3.46.

### b) 1-[5-Trifluoromethyl-2-(4-fluoro-benzyloxy)-phenyl]-pentane-1,4-dione

2-(4-Fluorobenzyloxy)-5-trifluoromethyl-benzaldehyde (14.00g, 0.047 mol, 1 eq) was dissolved in ethanol (7 ml) and methylvinylketone (3.98g, 0.047 mol, 1.02 eq), 3-ethyl-5-(2-hydroxyethyl)-4-methylthiazolium bromide (3.551 g, 0.014 mol, 0.3 eq) and Et₃N (14.23 g, 0.141 mol. 3 eq) were added to the stirred reaction mixture. The vessel was heated at reflux and stirred in a nitrogen atmosphere for 18 hours. The reaction mixture was quenched with sat. NH₄Cl solution and extracted with EtOAc, the combined organic extracts were washed with brine, dried (MgSO4), filtered and the volatiles were removed in vacuo. The residue was then purified by chromatography on silica gel with 10% EtOAc:iso-hexane as the eluant to yield the title compound (9.17g, 0.025 mol, 53%) as an off-white solid.

LC/MS t=3.58 min [MH⁻] 367.

### c) 3-{2-[5-Trifluoromethyl-2-(4-fluoro-benzyloxy)-phenyl]-5-methyl-pyrrol-1-yl}-6-chloro-benzoic acid

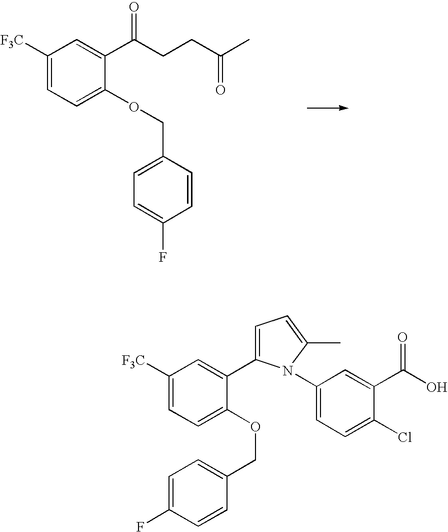

1-[5-Trifluoromethyl-2-(4-fluoro-benzyloxy)-phenyl]-pentane-1,4-dione (0.200g, 0.54 mmol, 1.00 eq) and pTSA (0.0305g, 0.16 mmol, 0.30 eq) were heated in a sealed vessel with stirring, at 150° C. for 540 seconds. The reaction mixture was allowed to warm to room temperature and the reaction mixture was diluted with ether, washed with 2M HCl solution and 2M sodium bicarbonate solution. The combined organic extracts were washed with brine and dried (MgSO₄), filtered and volatiles removed in vacuo. The residue was purified by chromatography on silica gel with 20% EtOAc:iso-hexane as the eluant to yield the title compound (0.122g, 45%) as yellow solid.

¹H NMR (400 MHz, d6-DMSO) 2.09 (3H, s), 4.96 (2H, s), 6.09 (1H, d, J=3 Hz), 6.30 (1H, d, J=3 Hz), 7.06 (1H, d, J=8 Hz), 7.13-7.24 (5H, m), 7.38 (1H, d, J=2 Hz), 7.41 (1H, d, J=3 Hz), 7.48 (1H, d, J=9 Hz), 7.54 (1H, dd, J=9 Hz, 2 Hz), 13.55 (1H, s).

LC/MS t=4.15 min [MH+] 504/506.

### Example 243

### 3-{2-[S-Trifluoromethyl-2-(4-fluoro-benzyloxy)-phenyl]-5-methyl-pyrrol-1-yl}-6-fluoro-benzoic acid

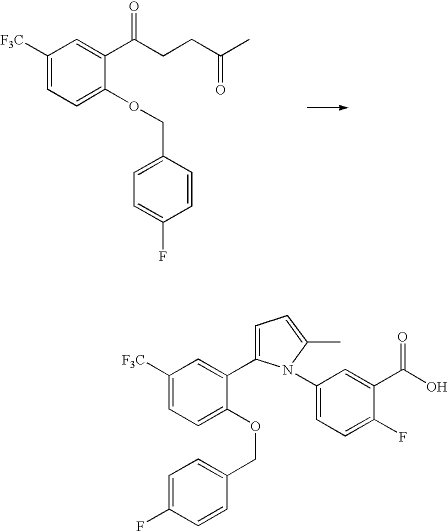

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, d6-DMSO) 2.08 (3H, s), 4.98 (2H, s), 6.08 (1H, d, J=3 Hz), 6.30 (1H, d, J=3 Hz), 7.08 (1H, d, J=8 Hz), 7.15-7.21 (2H, m), 7.23-7.35 (5H, m) 7.42-7.45 (1H, m), 7.53 (1H, dd, J=9 Hz, 2 Hz),13.35 (1H, s).

LC/MS t=4.01 min [MH+] 488.

### Example 244

### 3-{2-[5-Trifluoromethyl-2-(4-fluoro-benzyloxy)-phenyl]-5-methyl-pyrrol-1-yl}-4-fluoro-benzoic acid

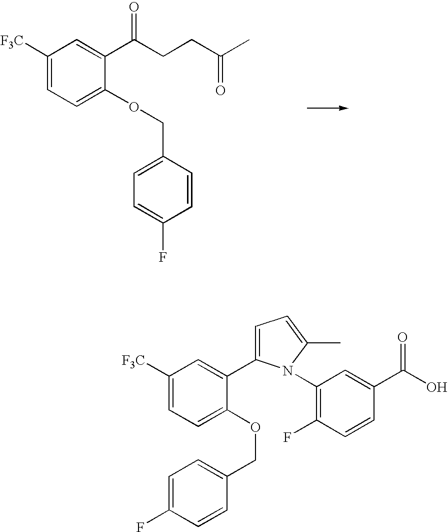

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=4.01 min [MH+] 488.

### Example 245

### 3-{2-[5-Trifluoromethyl-2-(4-fluoro-benzyloxy)-phenyl]-5-methyl-pyrrol-1-yl}-methyl-benzoic acid methyl ester

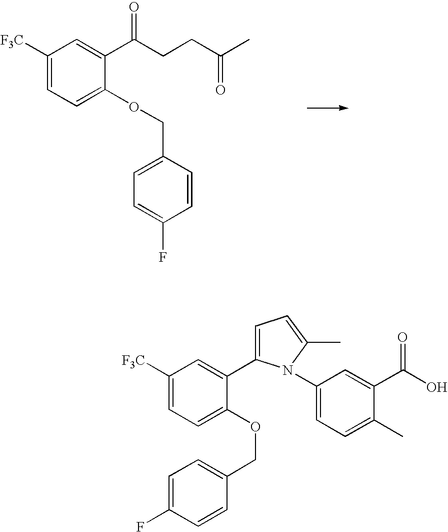

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, d6-DMSO) 2.06 (3H, s), 2.49 (3H, s), 4.98 (2H, s), 6.06 (1H, d, J=3 Hz), 6.28 (1H, d, J=3 Hz), 7.05 (1H, d, J=8H), 7.10-7.32 (7H, m), 7.43 (1H, d, J=3 Hz), 7.49 (1H, dd, J=8 Hz, 1 Hz), 12.80 (1H brs).

LC/MS t=3.84 min [MH+] 484.

### Example 246

### 3-{2-[5-Trifluoromethyl-2-(4-fluoro-benzyloxy)-phenyl]-5-methyl-pyrrol-1-yl}-5-acetylamino-benzoic acid

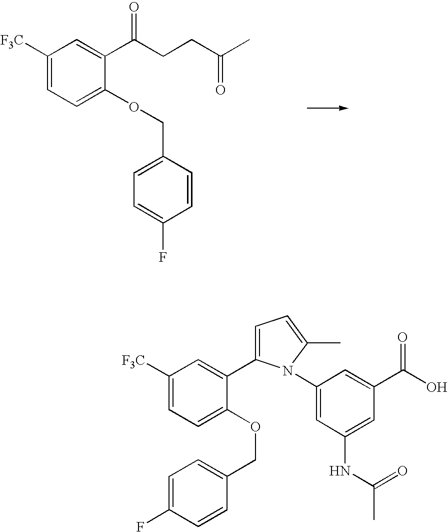

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.09 (3H, s), 2.13 (3H, s), 4.80 (2H, s), 6.11 (1H, d, J=3 Hz), 6.33 (1H, d, J=3 Hz), 6.69 (1H, d, J=9 Hz), 6.95 (2H, t, J=8 Hz), 7.03-7.12 (2H, m) 7.28 (1H, d, J=8 Hz,), 7.43 (2H, d, J=4 Hz), 7.60 (1H, s), 7.96 (1H, s,), 9.7 (1H, brs).

LC/MS t=3.52 [MH+] 527.

### Example 247

### 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid

### a) 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid methyl ester

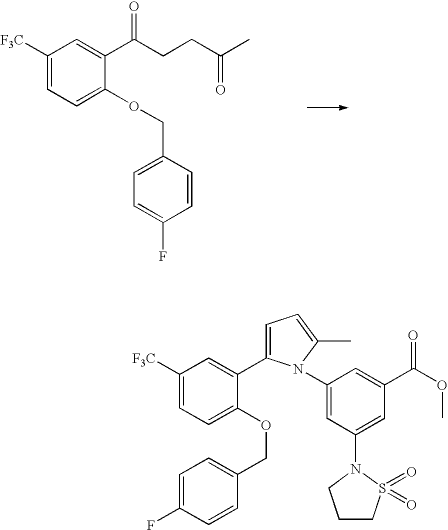

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=4.00 min [MH+] 603.

### b) 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid

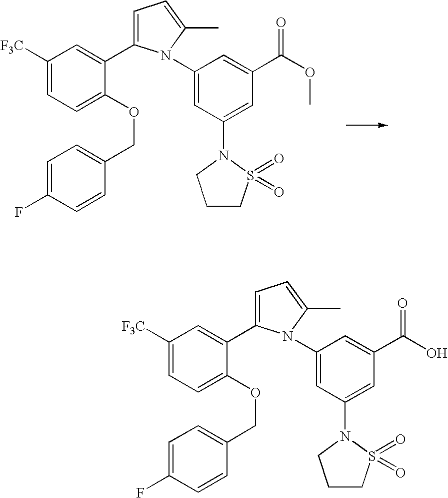

3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid methyl ester (0.20g, 0.33 mmol, 1 eq) was dissolved in ethanol (5 ml) and 2M NaOH (2 ml) was added. The reaction vessel was then heated with stirring to reflux for 2 hours. The reaction mixture was diluted with H₂O and extracted with ether. The aqueous extract was acidified with 2M HCl and extracted with ether and the combined organic extracts were washed with brine and dried (MgSO₄), filtered and the volatiles were removed in vacuo to yield the title compound (0.087g, 45%) as a yellow oil.

¹H NMR (400 MHz, CDCl₃) 2.22 (3H, s), 2.47 (2H, quint, J=7), 3.33 (2H, t, J=7), 3.48 (2H, t, J=7), 4.81 (2H, s), 6.16 (1H, d, J=3 Hz), 6.33 (1H, d, J=3 Hz), 6.73 (1H, d, J=9 Hz), 6.93-7.03 (3H, m), 7.50-7.11 (2H, m) 7.36 (1H, dd, J=8 Hz, 2 Hz), 7.48 (1H, d, J=2 Hz), 7.57 (1H, t, J=1 Hz) 7.84 (1H, t, J=1 Hz).

LC/MS t=3.57 min [MH+] 589.

### Example 248

### 3-{2-[5-Trifluoromethyl-2-(4-fluoro-benzyloxy)-phenyl]-5-methyl-pyrrol-1-yl}-5-(2-oxo-pyrrolidin-1-yl)-benzoic acid

### a) 3-{2-[5-Trifluoromethyl-2-(4-fluoro-benzyloxy)-phenyl]-5-methyl-pyrrol-1-yl}—(2-oxo-pyrrolidin-1-yl)-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=4.02 min [MH+] 567.

### b) 3-{2-[5-Trifluoromethyl-2-(4-fluoro-benzyloxy)-phenyl]-5-methyl-pyrrol-1-yl}-5-(2-oxo-pyrrolidin-1-yl)-benzoic acid

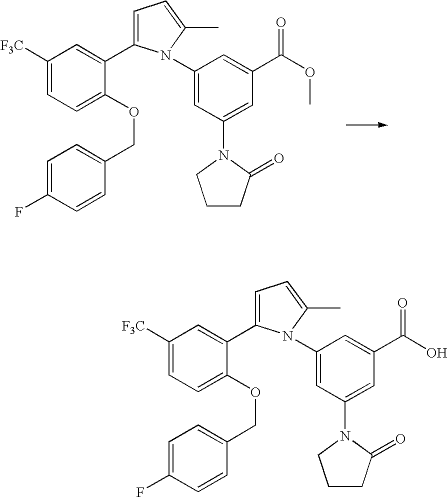

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

¹H NMR (400 MHz, CDCl₃) 2.22 (3H, s), 2.47 (2H, quint, J=7), 3.33 (2H, t, J=7), 3.48 (2H, t, J=7), 4.83 (2H, s), 6.16 (1H, d, J=3 Hz), 6.33 (1H, d, J=3 Hz), 6.74 (1H, d, J=8H), 6.97-7.08 (3H, m), 7.23-7.34 (4H, m), 7.39 (1H, dd, J=8 Hz, 1 Hz), 7.54 (1H, d, J=2 Hz), 7.65 (1H, d, J=2 Hz).

LC/MS t=3.62 min [MH+] 553.

### Example 249

### 3-{2-[5-Trifluoromethyl-2-(4-fluoro-benzyloxy)-phenyl]-5-methyl-pyrrol-1-yl}-5-(2-oxo-piperidin-1-yl)-benzoic acid methyl ester

### a) 3-{2-[5-Trifluoromethyl-2-(4-fluoro-benzyloxy)-phenyl]-5-methyl-pyrrol-1-yl}-5-(2-oxo-piperidin-1-yl)-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=3.96 min [MH+] 581.

### b) 3-{2-[5-Trifluoromethyl-2-(4-fluoro-benzyloxy)-phenyl]-5-methyl-pyrrol-1-yl}-5-(2-oxo-piperidin-1-yl)-benzoic acid methyl ester

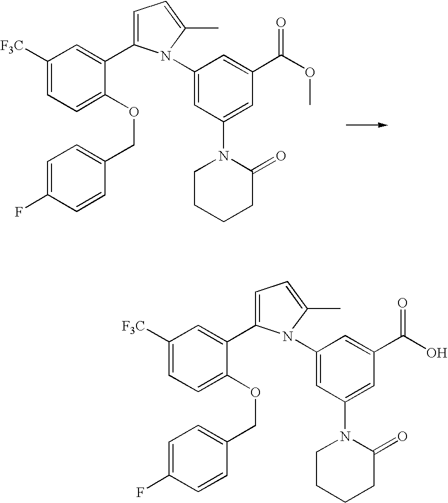

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

¹H NMR (400 MHz, d6-DMSO) 1.73-1.81 (2H m), 2.07-2.18 (5H, m), 2.34-2.40 (2H, m), 2.48-2.53 (2H, m), 4.94 (2H, s), 6.10 (1H, d, J=3 Hz), 6.31 (1H, d, J=3 Hz), 7.05 (1H, d, J=8 Hz), 7.07-7.28 (5H, m), 7.32 (1H, t, H=1), 7.41 (1H, d, J=2 Hz), 7.53 (1H, dd, J=8 Hz, 2 Hz), 7.78 (1H, t, J=1 Hz), 13.00 (1H, br s).

LC/MS t=3.56 min [MH+] 567.

### Example 250

### 3-{2-[5-Trifluoromethyl-2-(4-fluoro-benzyloxy)-phenyl]-5-methyl-pyrrol-1-yl}-5-amino-6-methyl-benzoic acid methyl ester

### a) 3-{2-[5-Trifluoromethyl-2-(4-fluoro-benzyloxy)-phenyl]-5-methyl-pyrrol-1-yl}-5-amino-6-methyl-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=3.96 min [MH+] 513.

### b) 3-{2-[5-Trifluoromethyl-2-(4-fluoro-benzyloxy)-phenyl]-5-methyl-pyrrol-1-yl}-5-amino-6-methyl-benzoic acid methyl ester

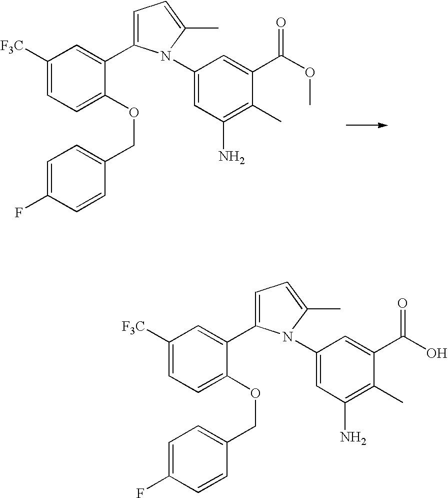

Procedure as for 3-{2-[5-Trifluoromethyl-2-(4-fluoro-benzyloxy)-phenyl]-5-methyl-pyrrol-1-yl)}-5-(1,1-dioxo-1I⁶-isothiazolidin-2-yl)-benzoic acid.

¹H NMR (400 MHz, CDCl₃) 2.16 (3H, s), 2.37 (3H, s), 3.71 (2H, s), 4.86 (2H, s), 6.11 (1H, d, J=3 Hz), 6.32 (1H, d, J=3 Hz), 6.42 (1H, d, J=2H), 6.74 (1H, d, J=8 Hz), 6.95-7.02 (2H, m), 7.06-7.14 (3H, m), 7.35 (1H, dd, J=8 Hz, 1 Hz), 7.48 (1H, d, J=2 Hz).

LC/MS. t=3.57 min [MH+] 499.

### Example 251

### 3-{2-[5-Trifluoromethyl-2-benzyloxy-phenyl]-5-methyl-pyrrol-1-yl}-6-fluoro-benzoic acid

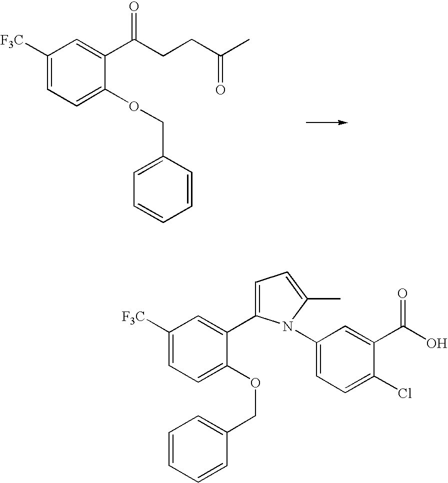

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.16 (3H, s), 4.83 (2H, s), 6.16 (1H, d, J=3 Hz), 6.33 (1H, d, J=3 Hz), 6.74 (1H, d, J=8H), 6.97-7.08 (3H, m), 7.23-7.34 (4H, m), 7.39 (1H, dd, J=8 Hz, 1 Hz), 7.54 (1H, d, J=2 Hz), 7.65 (1H, d, J=2 Hz).

LC/MS t=4.16 min [MH+] 486/488.

### Example 252

### 3-{2-[5-Trifluoromethyl-2-benzyloxy-phenyl]-5-methyl-pyrrol-1-yl}-4-fluoro-benzoic acid

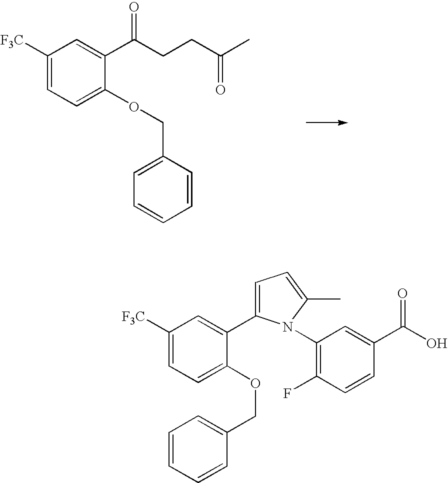

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.12(3H, s), 4.94 (2H, s), 6.18 (1H, d, J=3 Hz), 6.40 (1H, d, J=3 Hz), 6.72 (1H, d, J=9H), 7.07-7.15 (3H, m), 7.24-7.36 (4H, m), 7.45 (1H, d, J=2 Hz), 7.82 (1H, dd, J=8 Hz, 2 Hz), 8.01 (1H, m).

LC/MS t=3.99 min [MH+] 470.

### Example 253

### 3-{2-[5-Trifluoromethyl-2-benzyloxy-phenyl]-5-methyl-pyrrol-1-yl}-5-trifluoromethyl-benzoic acid

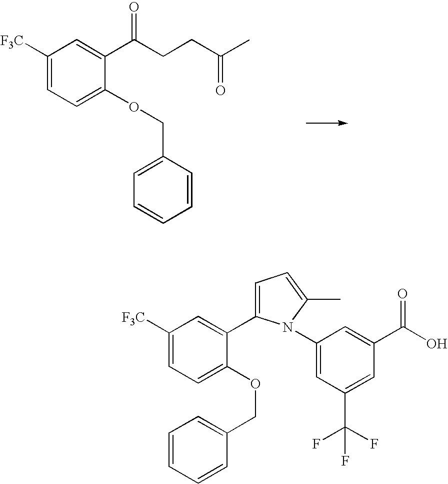

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.19 (3H, s), 4.81 (2H, s), 6.18 (1H, d, J=3 Hz), 6.38 (1H, d, J=3 Hz), 6.73 (1H, d, J=9H), 7.03-7.08 (2H, m), 7.24-7.32 (3H, m), 7.39 (1H, dd, J=8 Hz, 1 Hz), 7.44 (1H, s), 7.52 (1H, d, J=2 Hz), 7.89 (1H, s), 8.17 (1H, s).

LC/MS t=4.19 min [MH+] 520.

### Example 254

### 3-{2-[5-Trifluoromethyl-2-benzyloxy-phenyl]-5-methyl-pyrrol-1-yl}-5-amino-4-methyl-benzoic acid

### a) 3-{2-[5-Trifluoromethyl-2-benzyloxy-phenyl]-5-methyl-pyrrol-1-yl}-5-amino-6-methyl-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=4.03 min [MH+] 495.

### b) 3-{2-[5-Trifluoromethyl-2-benzyloxy-phenyl]-5-methyl-pyrrol-1-yl}-5-amino-6methyl-benzoic acid

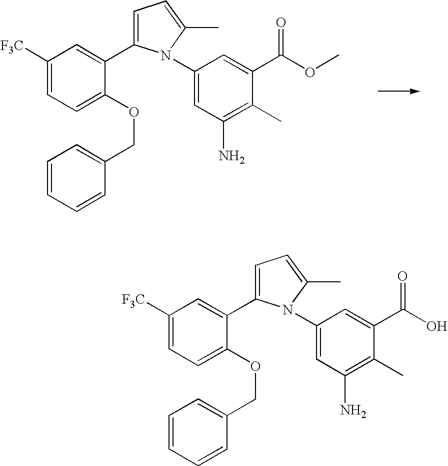

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

¹H NMR (400 MHz, CDCl₃) 2.15 (3H, s), 2.18 (2H, s), 2.36 (3H, s), 4.98 (2H, s), 6.11 (1H, d, J=3 Hz), 6.33 (1H, d, J=3 Hz), 6.42 (1H, d, J=2H) 6.73 (1H, d, J=8 Hz), 7.08-7.15 (3H, m), 7.25-7.36 (4H, m) 7.48 (1H, d, J=2 Hz,).

LC/MS t=3.71 min [MH+] 481.

### Example 255

### 3-{2-[5-Trifluoromethyl-2-(2,4-difluoro-benzyloxy)-phenyl]-5-methyl-pyrrol-1-yl}-6-chloro-benzoic acid

### a) 2-2,4-Difluoro-benzyloxy)-5-trifluoromethyl-benzaldehyde

Procedure as for 2-(4-Fluoro-benzyloxy)-5-trifluoromethyl-benzaldehyde using the appropriate benzyl bromide.

LC/MS t=3.74 min.

### b) 1-[5-Trifluoromethyl-2-(2,4-difluoro-benzyloxy)-phenyl]-pentane-1,4-dione

Procedure as for 1-[5-Trifluoromethyl-2-(4-fluoro-benzyloxy)-phenyl]-pentane-1,4-dione.

LC/MS t=3.62 min [MH−] 387.

### c) 3-{2-[5-Trifluoromethyl-2-(2,4-difluoro-benzyloxy)-phenyl]-5-methyl-pyrrol-1-yl}-6-chloro-benzoic acid

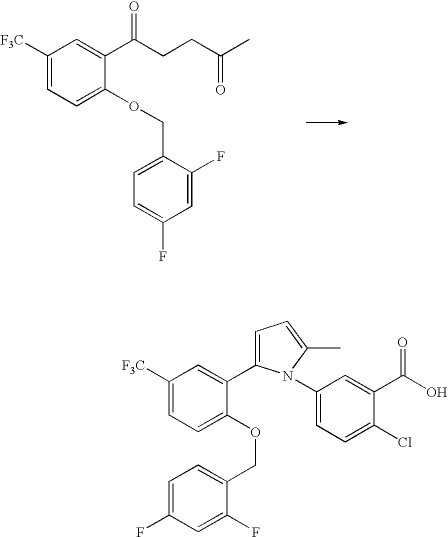

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.06 (3H, s), 4.88 (2H, s), 6.11 (1H, d, J=3 Hz), 6.33 (1H, d, J=3 Hz), 6.72-6.84 (3H, m), 7.08 (1H, dd, J=14 Hz, 8 Hz), 7.34 (1H, d, J=8 Hz) 7.42 (2H, d, J=6 Hz,), 7.72 (1H, s), 7.94 (1H, s), 9.80 (1H, s).

LC/MS t=3.89 min [MH+] 522/524.

### Example 256

### 3-{2-[5-Trifluoromethyl-2-(2,4-difluoro-benzyloxy)-phenyl]-5-methyl-pyrrol-1-yl}-6-fluoro-benzoic acid

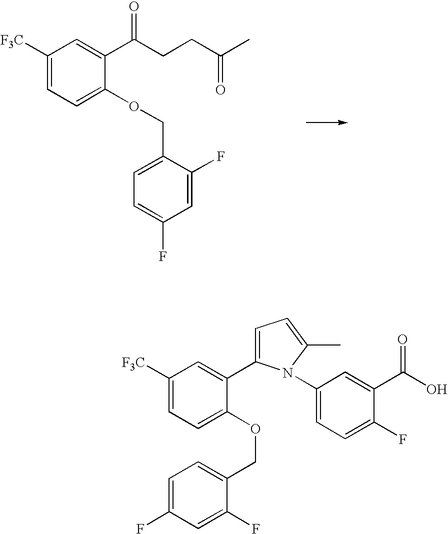

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.15 (3H, s), 4.88 (2H, s), 6.14 (1H, d, J=3 Hz), 6.33 (1H, d, J=3 Hz), 6.77-6.87 (3H, m), 6.98-7.16 (3H, m), 7.42 (1H, dd, J=9 Hz, 2 Hz) 7.48 (1H, d, J=2 Hz,), 7.69 (1H, dd, J=6 Hz, 3 Hz), 10.30 (1H, s).

LC/MS t=3.74 min [MH+] 506.

### Example 257

### 3-{2-[5-Trifluoromethyl-2-2,4-difluoro-benzyloxy)-phenyl]-5-methyl-pyrrol-1-yl}-4-fluoro-benzoic acid

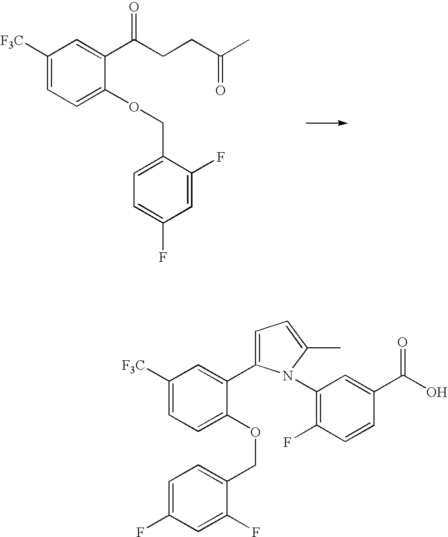

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.12 (3H, s), 4.94 (2H, s), 6.17 (1H, d, J=3 Hz), 6.38 (1H, d, J=3 Hz), 6.75-6.85 (3H, m), 7.06-7.16 (2H, m), 7.37 (1H, dd, J=9 Hz, 2 Hz) 7.44 (1H, d, J=2 Hz,), 7.78 (1H, dd, J=8 Hz, 2 Hz), 8.02 (1H, m), 9.80 (1H, br s).

LC/MS t=3.73 min [MH+] 506.

### Example 258

### 3-{2-[5-Trifluoromethyl-2-(2,4-difluoro-benzyloxy)-phenyl]-5-methyl-pyrrol-1-yl}-5-acetylamino-benzoic acid

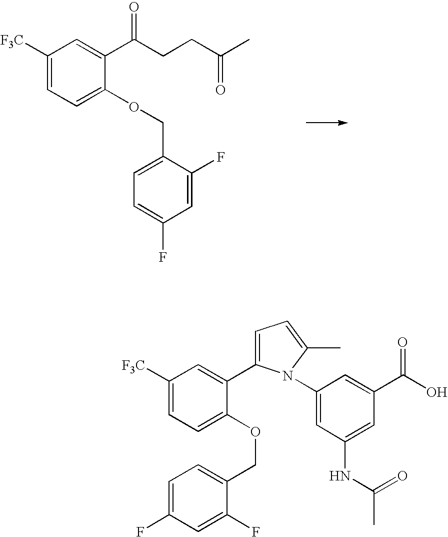

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.05 (3H, s), 2.16 (3H, s), 4.86 (2H, s), 6.15 (1H, d, J=3 Hz), 6.33 (1H, d, J=3 Hz), 6.74-6.88 (3H, m), 6.95-7.05 (2H, m), 7.32 (1H, d, J=8 Hz) 7.43 (1H, dd, J=9 Hz 2 Hz,), 7.53 (1H, s), 7.68 (1H, d, J=3 Hz), 10.60 (1H, s).

LC/MS t=3.53 min [MH+] 545.

### Example 259

### 3-{2-[5-Trifluoromethyl-2-(2,4-difluoro-benzyloxy)-phenyl]-5-methyl-pyrrol-1-yl}-5-(1,1-dioxo-1I⁶-isothiazolidin-2-yl}-benzoic acid

### a) 3-{2-[5-Trifluoromethyl-2-2,4-difluoro-benzyloxy)-phenyl]-5-methyl-pyrrol-1-yl}-5-(1,1-Dioxo-1I⁶-isothiazolidin-2-yl)-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=3.96 min [MH+] 621.

### b) 3-{2-[5-Trifluoromethyl-2-(2,4-difluoro-benzyloxy)-phenyl]-5-(1,1-dioxo-1I⁶-isothiazolidin-2-yl)-benzoic acid

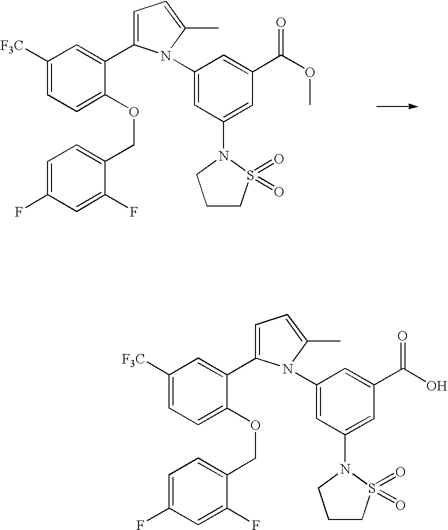

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid using the appropriate amine.

¹H NMR (400 MHz, CDCl₃) 2.20 (3H, s), 2.46 (2H, m), 3.32 (2H, t, J=8 Hz), 3.56 (2H, t, J=8 Hz), 4.86 (2H, s), 6.14 (1H, d, J=3 Hz), 6.34 (1H, d, J=3 Hz), 6.74-6.84 (3H, m), 7.02 (1H, dd, J=16 Hz, 8 Hz), 7.14 (1H, t, J=Hz), 7.40 (1H, dd J=9 Hz, 2 Hz), 7.45, (1H, s), 7.52 (1H, dd, J=9 Hz, 2 Hz), 7.69 (1H, dd, J=2 Hz, 1 Hz), 10.80 (1H, br s).

LC/MS t=3.58 min [MH+] 507.

### Example 260

### 3-{2-[5-Trifluoromethyl-2-(2,4-difluoro-benzyloxy)-phenyl]-5ethyl-pyrrol-1-yl}-5-(2-oxo-piperidin-1-yl)-benzoic acid

### a) 3-{2-[5-Trifluoromethyl-2-(2,4-difluoro-benzyloxy)-phenyl]-5-methyl-pyrrol-1-yl}-6-(2-oxo-piperidin-1-yl}-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=3.93 min [MH+] 599.

### b) 3-{2-[5-Trifluoromethyl-2-(2,4-difluoro-benzyloxy)-phenyl]-5-methyl-pyrrol-1-yl}-5-(2-oxo-piperidin-1-yl)-benzoic acid

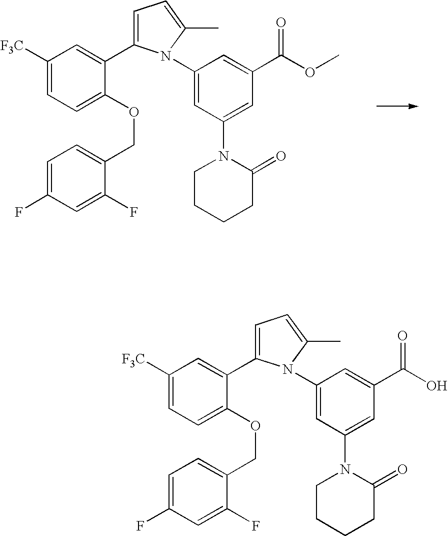

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

¹H NMR (400 MHz, CDCl₃) 1.80-1.90 (2H, m), 2.02-2.10 (2H, m), 2.21 (3H, s), 2.51-2.58 (2H, m), 3.25-3.32 (2H, m), 4.85 (2H, s), 6.12 (1H, d, J=3 Hz), 6.34 (1H, d, J=3 Hz), 6.76-6.87 (3H, m), 7.06-7.12 (2H, m), 7.40 (1H, dd, J=8 Hz, 2 Hz), 7.48 (1H, d, J=2 Hz), 7.58 (1H, t, J=1 Hz), 7.84 (1H, t, J=1 Hz), 11.25 (1H, br s).

LC/MS t=3.59 min [MH+] 585.

### Example 261

### 3-{2-[5-Trifluoromethyl-2-(2,4-difluoro-benzyloxy)-phenyl]-5-methyl-pyrrol-1-yl}-5-(methanesulfonyl)-benzoic acid

### a) 3-{2-[5-Trifluoromethyl-2-(2,4-difluoro-benzyloxy)-phenyl]-5-methyl-pyrrol-1-yl}-5-amino-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=3.97 min [MH+] 517.

### b) 3-{2-[6-Trifluoromethyl-2-2,4-difluoro-benzyloxy)-phenyl]-5-methyl-pyrrol-1-yl}-5-(methanesulfonyl)-benzoic acid methyl ester

3-{2-[5Trifluoromethyl-2-(2,4-difluoro-benzyloxy)-phenyl]-5-methyl-pyrrol-1-yl}-5-amino-benzoic acid methyl ester (0.0222g, 0.43 mmol, 1 eq), was dissolved in DCM (5 ml) and pyridine (0.0636 g, 0.86 mmol, 2 eq), 4-(dimethylamino) pyridine (0.057g, 0.47 mmol, 1.1 eq) and methylsulfonylchloride (0.03379, 0.29 mmol, 0.7 eq) were added. The reaction vessel was stirred for 18 hours at 21° C. The reaction mixture was diluted with DCM and washed with water. The combined organic extracts were washed with brine, dried (MgSO4), filtered and the volatiles were removed in vacuo to yield title compound (0.22g, 0.37 mmol, 86%) as a pale yellow solid.

LC/MS t=3.72 min [MH+] 595.

### c) 3-{2-[5-Trifluoromethyl-2-(2,4-difluoro-benzyloxy)-phenyl]-5-methyl-pyrrol-1-yl}-5-(methanesulfonyl)-benzoic acid

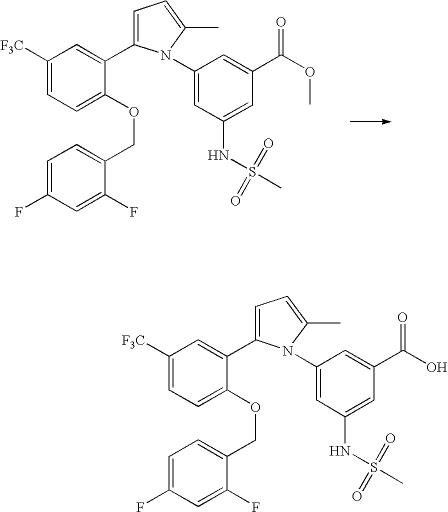

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

¹H NMR (400 MHz, d6-DMSO) 2.10 (3H, s), 2.70 (3H, s), 5.00 (2H, s), 6.08 (1H, d, J=3 Hz), 6.30 (1H, d, J=3 Hz), 7.20-7.80 (2H, m), 7.16-7.29 (5H, m), 7.53 (1H, dd, J=8 Hz, 2 Hz), 7.68 (1H, t, J=1 Hz), 10.00 (1H, s) 13.20 (1H, s).

LC/MS t=3.61 min [MH+] 581

### Example 262

### 4-{2-[5-Trifluoromethyl-2-(2,4-difluoro-benzyloxy)-phenyl]-6-methyl-pyrrol-1-yl}-2-methyl-benzoic acid

### a) 4-{2-[6-Trifluoromethyl-2-(2,4-difluoro-benzyloxy)-phenyl]-5-methyl-pyrrol-1-yl}-2-methyl-benzoic acid methyl ester

Procedure as for 3-{2-[5-Trifluoromethyl-2-(4-fluoro-benzyloxy)-phenyl]-5-methyl-pyrrol-1-yl}-6-chloro-benzoic acid using the appropriate amine, to give the title compound.

LC/MS t=3.84 min [MH+] 532.

### b) 4-{2-[5-Trifluoromethyl-2-(2,4-difluoro-benzyloxy)-phenyl]-5-methyl-pyrrol-1-yl}-2-methyl-benzoic acid

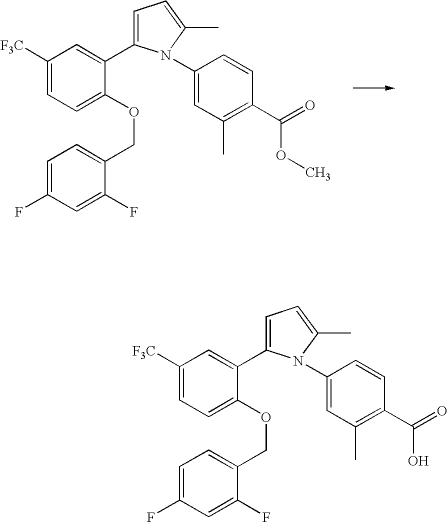

Procedure as for 3-{2-[5-Trifluoromethyl-2-(4-fluoro-benzyloxy)-phenyl]-5-methyl-pyrrol-1-yl}-5-(1,1-dioxo-1I⁶-isothiazolidin-2-yl)-benzoic acid.

LC/MS t=3.90 min [MH+] 518.

### Example 263

### 3-{2-[5-Chloro-2-(4-bromo-2-fluoro-benzyloxy)-phenyl]-5-methyl-pyrrol-1-yl}-5-hydroxy-benzoic acid

### a) 5-Chloro-2-(4-bromo-2-fluoro-benzyloxy)-benzaldehyde

5-Chlorosalicylaldehyde (5g, 32.05 mmol), 4-bromo-2-fluorobenzyl bromide (12.9g, 48.07 mmol) and K₂CO₃ (8.86g, 64.1 mmol) were heated in DMF (35 m], 1M) at 60° C. for 3 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (6.6 g, 60%).

¹H NMR (400 MHz, CDCl₃) 5.20 (2H, s), 7.03 (1H, d, J=9 Hz), 7.31-7.39 (3H, m), 7.51 (1H, dd, J=2.5 Hz, J=8.8 Hz), 7.82 (1H, d, J=2.5 Hz), 10.43 (1H, s).

### b) 1-[5-Chloro-2-(4-bromo-2-fluoro-benzyloxy)-phenyl]-pentane-1,4-dione

A mixture of 5-chloro-2-(4-bromo-2-fluoro-benzyloxy)-benzaldehyde (3.95g, 11.50 mmol), methyl vinyl ketone (1.2 ml, 14.38 mmol), 3ethyl-5-(2-hydroxyethyl)-4-methylthiazolium bromide (435 mg, 1.725 mmol, 0.1 5 eq) and triethylamine (2.81 ml, 20.125 mmol) was heated in EtOH (3.83 ml, 3M) at reflux for 7 hours. Upon cooling, the mixture was diluted with EtOAc and washed with sat. NH₄Cl and sat. NaHCO₃, dried (Na₂SO₄) filtered and concentrated. The residue was purified by chromatography, using Biotage, with cyclohexane containing a gradient of EtOAc (7.5-12.5%) to give the title compound (2.1g, 44%).

¹H NMR (400 MHz, CDCl₃) 2.21 (3H, s), 2.79-2.82 (2H, m), 3.18-3.21 (2H, m), 5.17 (2H, s), 6.97 (1H, d, J=9.0 Hz), 7.31-7.43 (4H, m), 7.70 (1H, d, J=2.8 Hz).

### c) 3-{2-[5-Chloro-2-(4-bromo-2-fluoro-benzyloxy)-phenyl]-5-methyl-pyrrol-1-yl}-5-hydroxy-benzoic acid methyl ester

1-[5-Chloro-2-(4-bromo-2-fluoro-benzyloxy)-phenyl]-pentane-1,4-dione (150 mg, 0.363 mmol), 3-amino-5-hydroxybenzoic acid methyl ester (61 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (112 mg, 57%).

¹H NMR (400 MHz, CDCl₃) 2.17 (3H, s), 3.85 (3H, s), 4.77 (2H, s), 6.12 (1H, d, J=3.3 Hz), 6.29 (1H, d, J=3.5 Hz), 6.61-6.63 (2H, m), 6.93 (1H, t, J=7.8 Hz), 7.09 (1H, dd, J=2.8 Hz, J=8.8 Hz), 7.21-7.23 (3H, m), 7.31 (1H, s), 7.39 (1H, s).

### d) 3-{2-[5-Chloro-2-(4-bromo-2-fluoro-benzyloxy)-phenyl]-5-methyl-pyrrol-1-yl}-5-hydroxy-benzoic acid

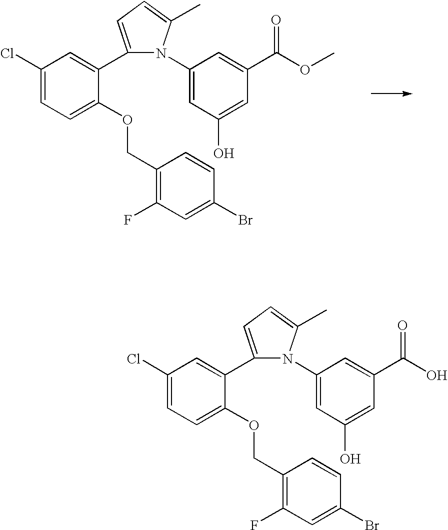

3-{2-[5-Chloro-2-(4-bromo-2-fluoro-benzyloxy)-phenyl]-5-methyl-pyrrol-1-yl}-5-hydroxy-benzoic acid methyl ester (112 mg, 0.21 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (57 mg, 51%).

¹H NMR (400 MHz, CDCl₃) 2.18 (3H, s), 4.78 (2H, s), 6.13 (1H, d, J=3.3 Hz), 6.30 (1H, d, J=3.3 Hz), 6.63 (1H, d, J=8.8 Hz), 6.69 (1H, bt, J=1.5 Hz), 6.94 (1H, t, J=8.0 Hz), 7.09 (1H, dd, J=2.5 Hz, J=8.8 Hz), 7.19-7.23 (3H, m), 7.37 (1H, s), 7.44 (1H, s).

LC/MS t=3.94 min, [MH+] 532 and 534, [MH−] 530 and 532.

### Example 264

### 3-{2-[5-Chloro-2-(4-bromo-2-fluoro-benzyloxy)-phenyl]-5-methyl-pyrrol-1-yl}-6-chloro-benzoic acid

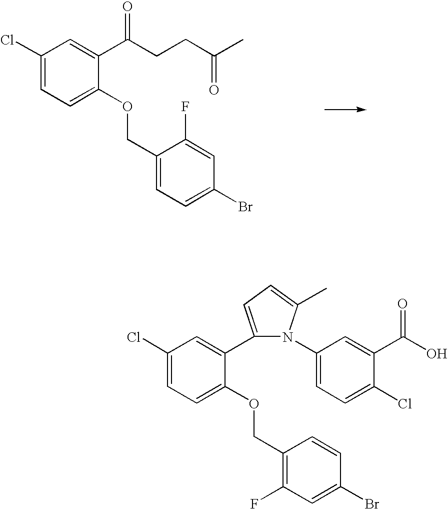

1-[5-Chloro-2-(4-bromo-2-fluorobenzyloxy)-phenyl]-pentane-1,4-dione (1 50 mg, 0.363 mmol), 3-amino-6-chlorobenzoic acid (62.3 mg, 0.363 mmol) and pTSA (5 mg) were heated in acetonitrile (2 ml) at 160° C. for 1 0 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (94 mg, 47%).

¹H NMR (400 MHz, CDCl₃) 2.17 (3H, s), 4.76 (2H, s), 6.14 (1H, s), 6.28 (1H, s), 6.62 (1H, d, J=9.0 Hz), 6.81-6.87 (1H, m), 7.03 (1H, d, J=8.5 Hz), 7.13 (1H, d, J=8.0 Hz), 7.20-7.29 (3H, m's excess), 7.33 (1H, d, J=8.5 Hz), 7.67 (1H, s).

LC/MS t=4.37 min, [MH+] 550 and 552, [MH−] 548 and 550.

### Example 265

### 3-{2-[5-Chloro-2-(4-bromo-2-fluoro-benzyloxy)-phenyl]-5methyl-pyrrol-1-yl}-6-fluoro-benzoic acid

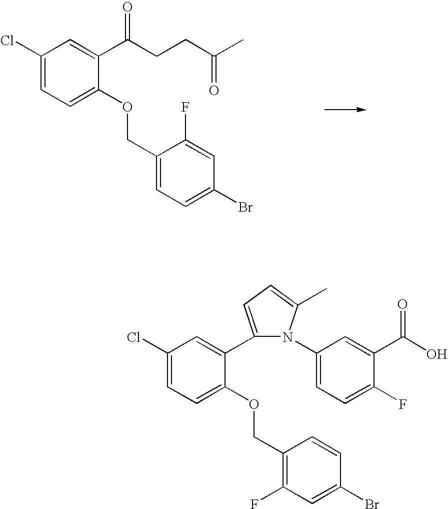

1-[5-Chloro-2-(4-bromo-2-fluoro-benzyloxy)-phenyl]-pentane-1,4-dione (1 50 mg, 0.363 mmol), 3-amino-6-fluorobenzoic acid (56.3 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (1 0-20%) to give the title compound (81 mg, 42%).

¹H NMR (400 MHz, CDCl₃) 2.15 (3H, s), 4.77 (2H, s), 6.14 (1H, s), 6.27 (1H, d, J=2.0 Hz), 6.63 (1H, d, J=8.8 Hz), 6.89-6.95 (1H, m), 6.99-7.06 (1H, m), 7.09-7.17 (2H, m), 7.21-7.27 (3H, m's excess), 7.69 (1H, d, J=5.8 Hz).

LC/MS t=4.14 min, [MH+] 534 and 536, [MH−] 532 and 534.

### Example 266

### 3-{2-[5-Chloro-2-(4-bromo-2-fluoro-benzyloxy)-phenyl]-5-methyl-pyrrol-1-yl}-4-fluoro-benzoic acid

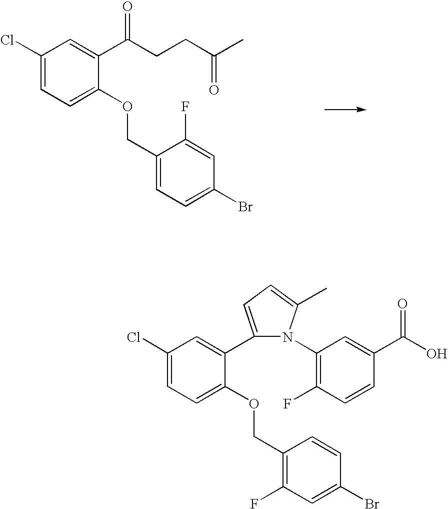

1-[5-Chloro-2-(4-bromo-2-fluoro-benzyloxy)-phenyl]-pentane-1,4-dione (150 mg, 0.363 mmol), 3-amino-fluorobenzoic acid (56.3 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (77 mg, 40%).

¹H NMR (400 MHz, CDCl₃) 2.12 (3H, s), 4.84 (2H, s), 6.17 (1H, s), 6.32 (1H, d, J=2.5 Hz), 6.59 (1H, d, J=8.5 Hz), 6.91-6.98 (1H, m), 7.04-7.10 (1H, m), 7.11-7.19 (1H, m), 7.19-7.24 (3H, m), 7.79 (1H, d, J=7.3 Hz), 8.00-8.06 (1H, m).

### Example 267

### 3-{2-[5-Chloro-2-(4-bromo-2-fluoro-benzyloxy)-phenyl]-5-methyl-pyrrol-1-yl}-5-acetylamino-benzoic acid

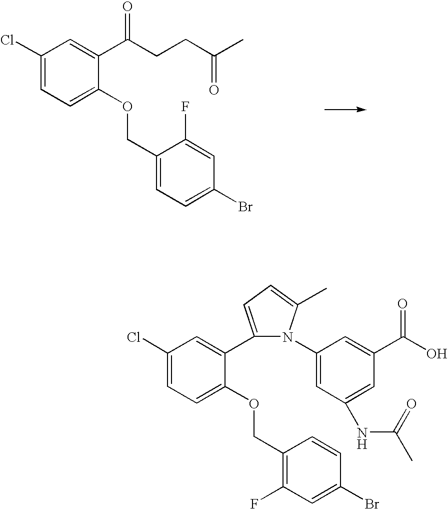

1-[5-Chloro-2-(4-bromo-2-fluoro-benzyloxy)-phenyl]-pentane-1,4-dione (150 mg, 0.363 mmol), 3-amino-5-acetylaminobenzoic acid (70.5 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (30-50%) to give the title compound (66 mg, 32%).

¹H NMR (400 MHz, CDCl₃) 2.18 (6H, s), 4.79 (2H, s), 6.13 (1H, s), 6.29 (1H, s), 6.60 (1H, d, J=9.0 Hz), 6.91-6.98 (1H, m), 7.07 (1H, d, J=8.5 Hz), 7.14-7.24 (3H, m), 7.47 (1H, s), 7.62 (1H, s), 7.95 (1H, s).

LC/MS t=3.82 min, [MH+] 573 and 575, [MH−] 571 and 573.

### Example 268

### 3-{2-[5-Chloro-2-(4-bromo-2-fluoro-benzyloxy)-phenyl]-5-methyl-pyrrol-1-yl}-1-naphthoic acid

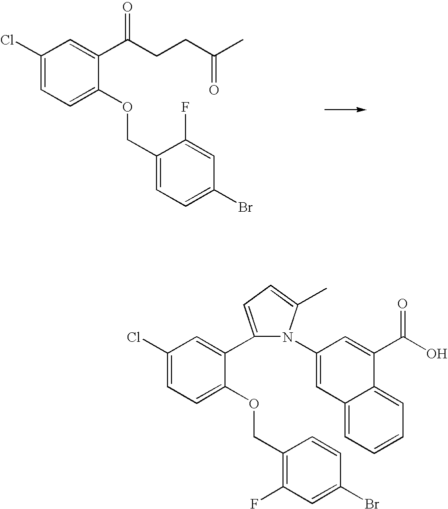

1-[5-Chloro-2-(4-bromo-2-fluoro-benzyloxy)-phenyl]-pentane-1,4-dione (150 mg, 0.363 mmol), 3-amino-1-naphthoic acid (67.9 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (98 mg, 48%).

¹H NMR (400 MHz, CDCl₃) 2.21 (3H, s), 4.69 (2H, s), 6.19 (1H, s), 6.33 (1H, s), 6.50 (1H, d, J=8.8 Hz), 6.64-6.70 (1H, m), 7.00 (1H, d, J=8.0 Hz), 7.06 (1H, d, J=9.0 Hz), 7.17 (1H, d, J=9.5 Hz), 7.38 (1H, s), 7.54 (1H, t, J=7.3 Hz), 7.61-7.70 (3H, m), 8.09 (1H, s), 9.04 (1H, d, J=8.8 Hz).

LC/MS t=4.30 min, [MH+] 566 and 568, [MH−] 564 and 566.

### Example 269

### 3-{2-[5-Chloro-2-(4bromo-2-fluoro-benzyloxy)-phenyl]-5-methyl-pyrrol-1-yl}-6-methyl-benzoic acid

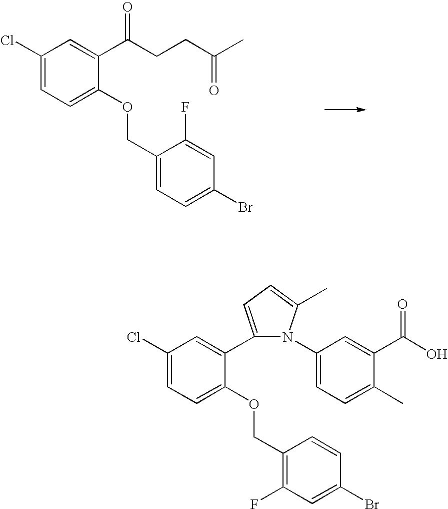

1-[5-Chloro-2-(4-bromo-2-fluoro-benzyloxy)-phenyl]-pentane-1,4dione (150 mg, 0.363 mmol), 3-amino-6-methylbenzoic acid (55 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (119 mg, 62%).

¹H NMR (400 MHz, CDCl₃) 2.16 (3H, s), 2.63 (3H, s), 4.76 (2H, s), 6.13 (1H, d, J=3.3 Hz), 6.29 (1H, d, J=3.5 Hz), 6.60 (1H, d, J=8.8 Hz), 6.88 (1H, t, J=7.8 Hz), 7.03 (1H, dd, J=2.3 Hz, J=8.0 Hz), 7.06-7.15 (2H, m), 7.19 (1H, s), 7.21 (1H, s), 7.24 (1H, d, J=2.5 Hz), 7.75 (1H, d, J=2.0 Hz).

LC/MS t=4.19 min, [MH+] 530 and 532, [MH−] 528 and 530.

### Example 270

### 3-{2-[5-Chloro-2-(4-bromo-2-fluoro-benzyloxy)-phenyl]-5-methyl-pyrrol-1-yl}-6-hydroxy-benzoic acid

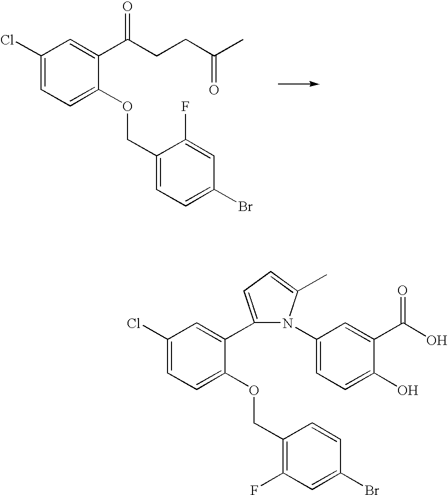

1-[5-Chloro-2-(4-bromo-2-fluoro-benzyloxy)-phenyl]-pentane-1,4-dione (150 mg, 0.363 mmol), 3amino-6-hydroxybenzoic acid (56 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (11 0 mg, 57%).

¹H NMR (400 MHz, CDCl₃) 2.15 (3H, s), 4.82 (2H, s), 6.12 (1H, d, J=3.3 Hz), 6.27 (1H, d, J=3.3 Hz), 6.64 (1H, d, J=8.8 Hz), 6.87 (1H, d, J=8.8 Hz), 6.91-6.97 (1H, m), 7.09-7.14 (2H, m), 7.20-7.25 (3H, m), 7.56 (1H, d, J=2.5 Hz), 10.34 (1H, bs).

LC/MS t=4.93 min, [MH+] 532 and 534, [MH−] 530 and 532.

### Example 271

### 3-{2-[5-Chloro-2-(4-bromo-2-fluoro-benzyloxy)-phenyl]-5-methyl-pyrrol-1-yl}-5-bromo-benzoic acid

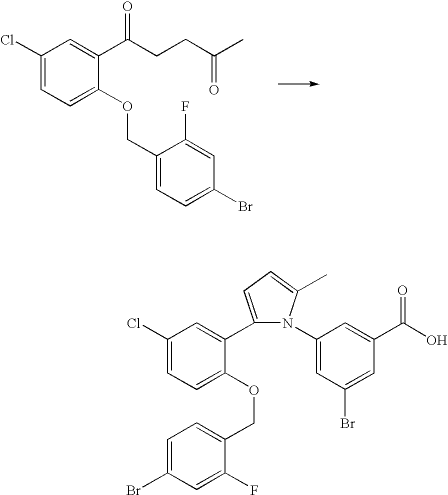

1-[5-Chloro-2-(4-bromo-2-fluoro-benzyloxy)-phenyl]-pentane-1,4-dione (1 50 mg, 0.363 mmol), 3-amino-5-bromobenzoic acid (79 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (45 mg, 21%).

¹H NMR (400 MHz, CDCl₃) 2.17 (3H, s), 4.76 (2H, s), 6.13 (1H, d, J=4.0 Hz), 6.28 (1H, d, J=3.5 Hz), 6.64 (1H, d, J=8.8 Hz), 6.89-6.95 (1H, m), 7.13 (1H, dd, J=2.5 Hz, J=8.8 Hz), 7.24-7.28 (3H, m's excess), 7.33-7.36 (1H, m), 7.63-7.66 (1H, m), 8.09 (1H, t, J=1.5 Hz).

LC/MS t=4.49 min, [MH+] 594 and 596, [MH−] 592 and 594.

### Example 272

### 3-{2-[5-Chloro-2-(4-bromo-2-fluoro-benzyloxy)-phenyl]-5-methyl-pyrrol-1-yl}-5-amino-benzoic acid

### a) 3-{2-[5-Chloro-2-(4-bromo-2-fluoro-benzyloxy)-phenyl]-5-methyl-pyrrol-1-yl}-5-amino-benzoic acid methyl ester

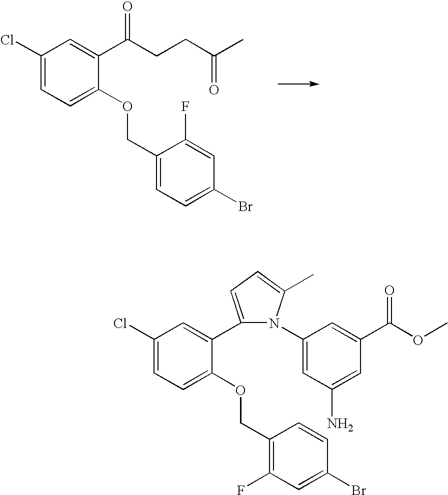

1-[5-Chloro-2-(4-bromo-2-fluoro-benzyloxy)-phenyl]-pentane-1,4-dione (150 mg, 0.363 mmol), 3,5-diaminobenzoic acid methyl ester (60.4 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (60 mg, 30%).

¹H NMR (400 MHz, CDCl₃) 2.15 (3H, s), 3.70 (2H, bs), 3.82 (3H, s), 4.79 (2H, s), 6.10 (1H, s), 6.29 (1H, s), 6.42 (1H, s), 6.62 (1H, d, J=8.8 Hz), 6.96 (1H, t, J=7.8 Hz), 7.08 (1H, d, J=8.5 Hz), 7.12 (1H, s), 7.18-7.24 (4H, m).

### b) 3-{2-[5-Chloro-2-(4-bromo-2-fluoro-benzyloxy)-phenyl]-5-methyl-pyrrol-1-yl}-5-amino-benzoic acid

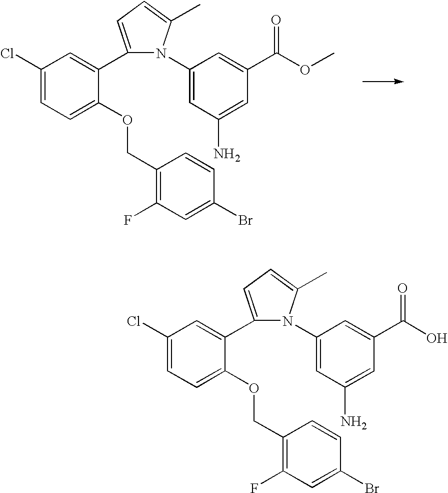

3-{2-[5-Chloro-2-(4-bromo-2-fluoro-benzyloxy)-phenyl]-5-methyl-pyrrol-1-yl}-5-amino-benzoic acid methyl ester (60 mg, 0.1 1 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (43 mg, 74%).

¹H NMR (400 MHz, CDCl₃) 2.14 (3H, s), 4.89 (2H, s), 6.04 (1H, d, J=2.8 Hz), 6.29 (1H, d, J=3.3 Hz), 6.49 (1H, s), 6.83-6.93 (3H, m), 7.06-7.11 (2H, m), 7.21 (1H, s), 7.26-7.32 (2H, m's excess).

LC/MS t=3.81 min, [MH+] 531 and 533, [MH−] 529 and 531.

### Example 273

### 3-{2-[5-Chloro-2-(4-bromo-2-fluoro-benzyloxy)-phenyl]-5-methyl-pyrrol-1-yl}-6-acetylamino-benzoic acid

### a) 3-{2-[5-Chloro-2-(4-bromo-2-fluoro-benzyloxy)-phenyl]-5-methyl-pyrrol-1-yl}-6-acetylamino-benzoic acid methyl ester

1-[5-Chloro-2-(4-bromo-2-fluoro-benzyloxy)-phenyl]-pentane-1,4-dione (150 mg, 0.363 mmol), 3-amino-6-acetylaminobenzoic acid methyl ester (76 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (1 29 mg, 61%).

¹H NMR (400 MHz, CDCl₃) 2.13 (3H, s), 2.24 (3H, s), 3.81 (3H, s), 4.75 (2H, s), 6.11 (1H, d, J=3.3 Hz), 6.26 (1H, d, J=3.5 Hz), 6.61 (1H, d, J=8.8 Hz), 6.88 (1H, t, J=8.0 Hz), 7.08-7.15 (2H, m), 7.19-7.25 (2H, m), 7.26-7.29 (1H, m's excess), 7.67 (1H, d, J=2.5 Hz), 8.60 (1H, d, J=9.0 Hz), 11.00 (1H, bs).

### b) 3-{2-[5-Chloro-2-(4-bromo-2-fluoro-benzyloxy)-phenyl]-5-methyl-pyrrol-1-yl}-6-acetylamino-benzoic acid

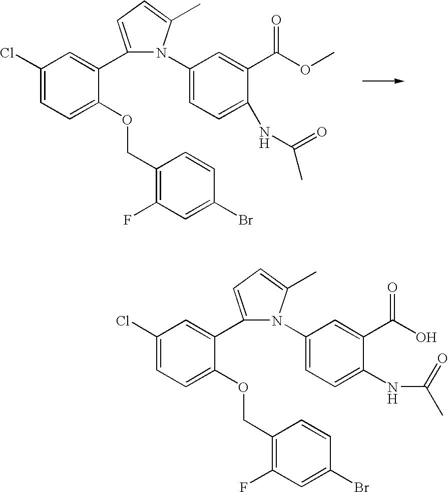

3-{2-[5-Chloro-2-(4-bromo-2-fluoro-benzyloxy)-phenyl]-5-methyl-pyrrol-1-yl}-6-acetylamino-benzoic acid methyl ester (129 mg, 0.22 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (71.3 mg, 57%).

¹H NMR (400 MHz, CDCl₃) 2.15 (3H, s), 2.26 (3H, s), 4.77 (2H, s), 6.13 (1H, d, J=3.3 Hz), 6.27 (1H, d, J=3.3 Hz), 6.61 (1H, d, J=8.5 Hz), 6.88 (1H, t, J=8.0 Hz), 7.10 (1H, dd, J=2.5 Hz, J=8 Hz), 7.16-7.24 (3H, m), 7.25-7.29 (1H, m's excess), 7.74 (1H, d, J=2.5 Hz), 8.63 (1H, d, J=8.8 Hz), 10.85 (1H, bs).

LC/MS t=31 min, [MH+] 573 and 575, [MH−] 571 and 573.

### Example 274

### 3-{2-[5-Chloro-2-(4-bromo-2-fluoro-benzyloxy)-phenyl]-5-methyl-pyrrol-1-yl}-5-trifluoromethyl-benzoic acid

### a) 3-{2-[5-Chloro-2-(4-bromo-2-fluoro-benzyloxy)-phenyl]-5-methyl-pyrrol-1-yl}-5-trifluoromethyl-benzoic acid methyl ester

1-[5-Chloro-2-(4-bromo-2-fluoro-benzyloxy)-phenyl]-pentane-1,4dione (150 mg, 0.363 mmol), 3-amino-5-trifluoromethyl-benzoic acid methyl ester (80 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (0-5%) to give the title compound (78 mg, 36%).

¹H NMR (400 MHz, CDCl₃) 2.18 (3H, s), 3.90 (3H, s), 4.70 (2H, s), 6.16 (1H, d, J=3.0 Hz), 6.30 (1H, d, J=3.3 Hz), 6.60 (1H, d, J=8.8 Hz), 6.85-6.91 (1H, m), 7.10-7.15 (1H, m), 7.21 (1H, s), 7.23 (1H, s), 7.26-7.29 (1H, m's excess), 7.39 (1H, s), 7.84 (1H, s), 8.15 (1H, s).

### b) 3-{2-[5-Chloro-2-(4-bromo-2-fluoro-benzyloxy)-phenyl]-5-methyl-pyrrol-1-yl}-5-trifluoromethyl-benzoic acid

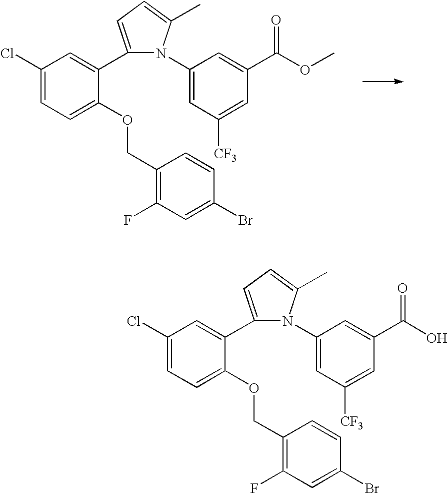

3-{2-[5-Chloro-2-(4-bromo-2-fluoro-benzyloxy)-phenyl]-5-methyl-pyrrol-1-yl}-5-trifluoromethyl-benzoic acid methyl ester (78 mg, 0.13 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (61 mg, 81%).

¹H NMR (400 MHz, CDCl₃) 2.20 (3H, s), 4.71 (2H, s), 6.18 (1H, d, J=3.5 Hz), 6.31 (1H, d, J=3.5 Hz), 6.62 (1H, d, J=9.0 Hz), 6.89 (1H, t, J=8.3 Hz), 7.14 (1H, dd, J=2.5 Hz, J=8.8 Hz), 7.19-7.25 (2H, m), 7.29 (1H, d, J=2.8 Hz), 7.45 (1H, s), 7.90 (1H, s), 8.22 (1H, s).

### Example 275

### 3-{2-[5-Chloro-2-(4-bromo-2-fluoro-benzyloxy)-phenyl]-5-methyl-pyrrol-1-yl}-5-(1,1-dioxo-If-isothiazolidin-2-yl)-benzoic acid

### a) 3-{2-[5-Chloro-2-(4-bromo-2-fluoro-benzyloxy)-phenyl]-methyl-pyrrol-1-yl}-5-(1,1-dioxo-1I⁶-isothiazolidin-2-yl)-benzoic acid methyl ester

1-[5-Chloro-2-(4-bromo-2-fluoro-benzyloxy)-phenyl]-pentane-1,4-dione (150 mg, 0.363 mmol), 3-amino-5-(1,1-dioxo-1I⁶-isothiazolidin-2-yl)-benzoic acid methyl ester (98 mg, 0.363 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (30-50%) to give the title compound (152 mg, 65%).

¹H NMR (400 MHz, CDCl₃) 2.23 (3H, s), 2.52 (2H, t, J=6.5 Hz), 3.30-3.38 (2H, m), 3.52-3.59 (2H, m), 3.86 (3H, s), 4.77 (2H, s), 6.12 (1H, s), 6.27 (1H, s), 6.60 (1H, d, J=8.3 Hz), 6.87 (1H, t, J=7.8 Hz), 7.06-7.23 (4H, m), 7.24-7.29 (1H, m's excess), 7.45 (1H, s), 7.68 (1H, s).

### b) 3-{2-[5-Chloro-2-(4-bromo-2-fluoro-benzyloxy)-phenyl]-5-methyl-pyrrol-1-yl}-(1,1-dioxo-1I⁶-isothiazolidin-2-yl)-benzoic acid

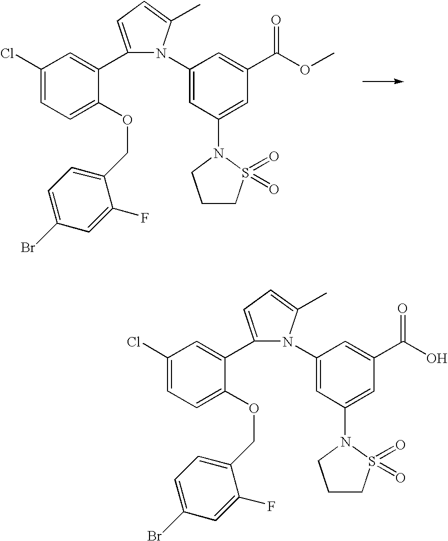

3-{2-[5-Chloro-2-(4-bromo-2-fluoro-benzyloxy)-phenyl]-5-methyl-pyrrol-1-yl}-5-(1,1-dioxo-1I⁶-isothiazolidin-2-yl)-benzoic acid methyl ester (152 mg, 0.23 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (74.5 mg, 51%).

¹H NMR (400 MHz, CDCl₃) 2.21 (3H, s), 2.46-2.56 (2H, m), 3.32-3.38 (2H, m), 3.58 (2H, t, J=6.5 Hz), 4.79 (2H, s), 6.11-6.15 (1H, m), 6.27-6.29 (1H, m), 6.57-6.63 (1H, m), 6.82-6.91 (1H, m), 7.07-7.13 (1H, m), 7.18-7.23 (3H, m), 7.25-7.28 (1H, m's excess), 7.46-7.50 (1H, m), 7.66-7.72 (1H, m).

### Example 276

### 3-{2-[5-Chloro-2-(2,6-difluoro-benzyloxy)-phenyl]-5-methyl-pyrrol-1-yl}-5-hydroxy-benzoic acid

### a) 5-Chloro-2-(2,6-difluoro-benzyloxy)-benzaldehyde

5-Chlorosalicylaldehyde (5g, 32.05 mmol), 2,6-difluorobenzyl bromide (6.64g, 32.05 mmol) and K₂CO₃ (8.86g, 64.1 mmol) were heated in DMF (35ml, 1M) at 60° C. for 3 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (7.9g, 87%).

¹H NMR (400 MHz, CDCl₃) 5.25 (2H, s), 6.93-6.99 (2H, m), 7.14 (1H, d, J=9.0 Hz), 7.33-7.41 (1H, m), 7.51 (1H, dd, J=2.8 Hz, J=9.0 Hz), 7.77 (1H, d, J=2.8 Hz), 10.32 (1H, s).

### b) 1-[5-Chloro-2-(2,6-difluoro-benzyloxy)-phenyl]-pentane-1,4-dione

A mixture of 5-chloro-2-(2,6-difluoro-benzyloxy)-benzaldehyde (7.9g, 28 mmol), methyl vinyl ketone (2.45 ml, 29.41 mmol), 3-ethyl-5-(2-hydroxyethyl)-4-methylthiazolium bromide (1.06g, 4.2 mmol, 0.15 eq) and triethylamine (4.9 ml, 35 mmol) was heated in EtOH (9.3 ml, 3M) at 80° C. for 5 hours. Upon cooling, the mixture was diluted with EtOAc and washed with sat. NH₄Cl and sat. NaHCO₃, dried (Na₂SO₄) filtered and concentrated. The residue was purified by chromatography, using Biotage®, with cyclohexane containing a gradient of EtOAc (5-15%) to give the title compound (3.01g, 30.5%).

¹H NMR (400 MHz, CDCl₃) 2.15 (3H, s), 2.72-2.75(2H, m), 3.11-3.15 (2H, m), 5.22 (2H, s), 6.92-6.99 (2H, m's, excess), 7.09 (1H, d, J=9.0 Hz), 7.33-7.44 (2H, m's, excess), 7.69 (1H, d, J=2.8 Hz).

### c) 3-{2-[5-Chloro-2-(2,6-difluoro-benzyloxy)-phenyl]-5-methyl-pyrrol-1-yl}-5-hydroxy-benzoic acid methyl ester

1-[5-Chloro-2-(2,6-difluoro-benzyloxy)-phenyl]-pentane-1,4-dione (150 mg, 0.43 mmol), 3-amino-5-hydroxybenzoic acid methyl ester (71 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (68 mg, 33%).

¹H NMR (400 MHz, CDCl₃) 2.13 (3H, s), 3.88 (3H, s), 4.87 (2H, s), 6.05 (1H, d, J=3.5 Hz), 6.28 (1H, d, J=3.5 Hz), 6.62-6.64 (1H, m), 6.83-6.91 (3H, m), 7.06-7.12 (2H, m), 7.28-7.34 (2H, m), 7.40-7.41 (1H, m).

### d) 3-{2-[5-Chloro-2-(2,6-difluoro-benzyloxy)-phenyl]-5-methyl-pyrrol-1-yl}-5-hydroxy-benzoic acid

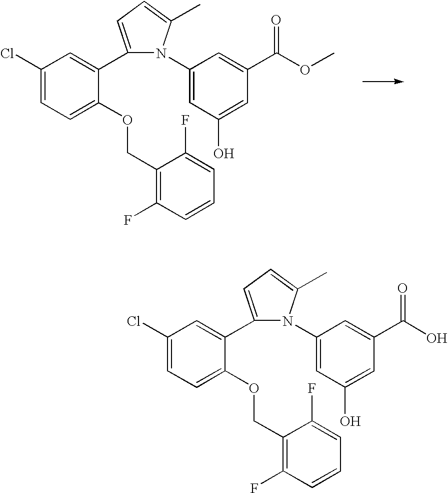

3-{2-[5-Chloro-2-(2,6-difluoro-benzyloxy)-phenyl]-5-methyl-pyrrol-1-yl}-5-hydroxy-benzoic acid methyl ester (68 mg, 0.14 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (37 mg, 56%).

¹H NMR (400 MHz, d6 DMSO) 2.02 (3H, s), 4.91 (2H, s), 5.97 (1H, d, J=4.0 Hz), 6.16 (1H, d, J=3.5 Hz), 6.57 (1H, t, J=2.0 Hz), 6.92 (1H, d, J=2.8 Hz), 6.95 (1H, t, J=1.5 Hz), 7.09-7.14 (3H, m), 7.20-7.25 (2H, m), 7.46-7.53 (1H, m), 9.96 (1H, s).

LC/MS t=3.68 min, [MH+] 470 and 472, [MH−] 468 and 470.

### Example 277

### 3-{2-[5-Chloro-2-(2,6-difluoro-benzyloxy)-phenyl]-5-methyl-pyrrol-1-yl}-6-hydroxy-benzoic acid

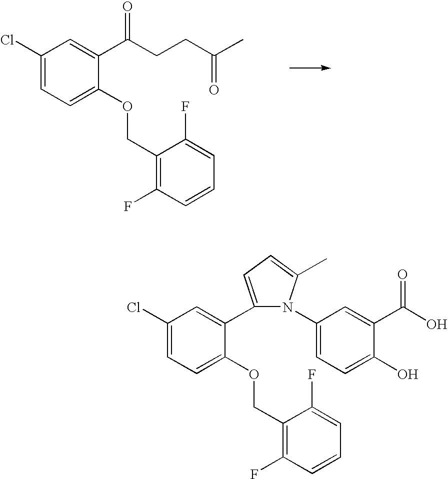

1-[5-Chloro-2-(2,6-difluoro-benzyloxy)-phenyl]-pentane-1,4-dione (200 mg, 0.57 mmol), 3-amino-6-hydroxybenzoic acid (87 mg, 0.57 mmol) and pTSA (5 mg) were heated in acetonitrile (2.5 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-30%) to give the title compound (95 mg, 36%).

¹H NMR (400 MHz, CDCl₃) 2.10 (3H, s), 4.86 (2H, s), 6.04 (1H, d, J=3.3 Hz), 6.24 (1H, d, J=3.5 Hz), 6.81-6.93 (4H, m), 7.08-7.13 (3H, m), 7.26-7.33 (1H, m's excess), 7.55 (1H, d, J=2.5 Hz), 10.52 (1H, bs).

### Example 278

### 3-{2-[5-Chloro-2-(2,6-difluoro-benzyloxy)-phenyl]-5-methyl-pyrrol-1-yl}-6-chloro-benzoic acid

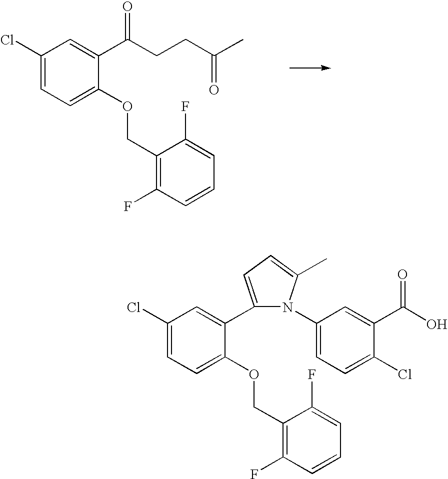

1-[5-Chloro-2-(2,6-difluoro-benzyloxy)-phenyl]-pentane-1,4-dione (150 mg, 0.43 mmol), 3-amino-6-chlorobenzoic acid (73.3 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (1 5-30%) to give the title compound (98 mg, 48%).

¹H NMR (400 MHz, CDCl₃) 2.14 (3H, s), 4.81 (2H, s), 6.08 (1H, d, J=3.3 Hz), 6.23 (1H, d, J=3.3 Hz), 6.87 (3H, m), 7.02 (1H, dd, J=2.5 Hz, J=8.5 Hz), 7.13-7.20 (2H, m), 7.25-7.35 (2H, m's excess), 7.70 (1H, d, J=2.5 Hz).

### Example 279

### 3-{2-[5-Chloro-2-(2,6-difluoro-benzyloxy)-phenyl]-5-methyl-pyrrol-1-yl}-6-fluoro-benzoic acid

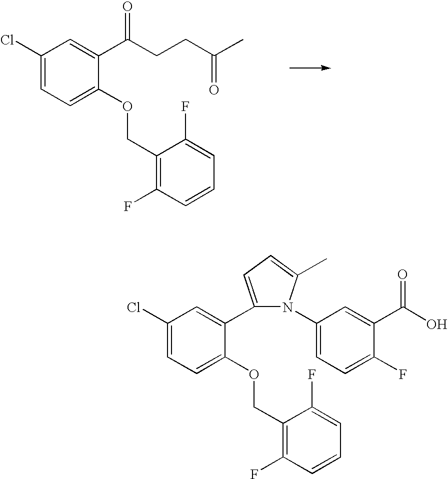

1-[5-Chloro-2-(2,6difluoro-benzyloxy)-phenyl]-pentane-1,4-dione (150 mg, 0.43 mmol), 3-amino-6-fluorobenzoic acid (66 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (46 mg, 23%).

¹H NMR (400 MHz, CDCl₃) 2.12 (3H, s), 4.82 (2H, s), 6.07 (1H, s), 6.23 (1H, d, J=2.5 Hz), 6.83 (1H, d, J=9.3 Hz), 6.90 (2H, t, J=7.8 Hz), 7.02 (1H, t, J=9.0 Hz), 7.13 (3H, m), 7.31 (1H, m's excess), 7.68 (1H, m).

LC/MS t=3.85 min [MH+] 472, [MH−] 470.

### Example 280

### 3-{2-[5-Chloro-2-(2,6-difluoro-benzyloxy)-phenyl]-5-methyl-pyrrol-1-yl}-5-acetylamino-benzoic acid

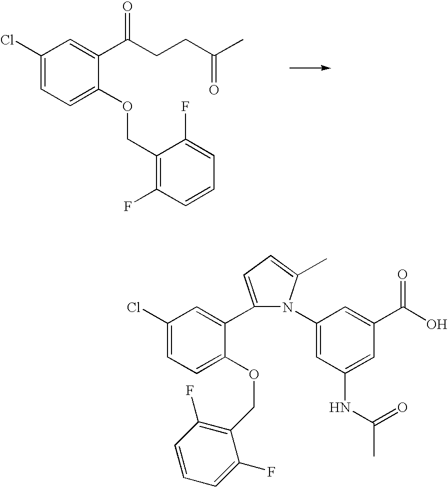

1-[5-Chloro-2-(2,6difluoro-benzyloxy)-phenyl]-pentane-1,4-dione (150 mg, 0.43 mmol), 3-amino-5-acetylaminobenzoic acid (83 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (30-50%) to give the title compound (48 mg, 22%).

¹H NMR (400 MHz, CDCl₃) 2.13 (3H, s), 2.19 (3H, s), 4.84 (2H, s), 6.06 (1H, S), 6.26 (1H, s), 6.77 (1H, d, J=8.8 Hz), 6.86 (2H, t, J=7.8 Hz), 7.06 (1H, d, J=8.8 Hz), 7.12 (1H, s), 7.43-7.61 (3H, m), 8.05 (1H, s).

LC/MS t=3.53 min [MH+] 511, [MH−] 509

### Example 281

### 3-{2-[5-Chloro-2-(2,6-difluoro-benzyloxy)-phenyl]-5-methyl-pyrrol-1-yl}-1-naphthoic acid

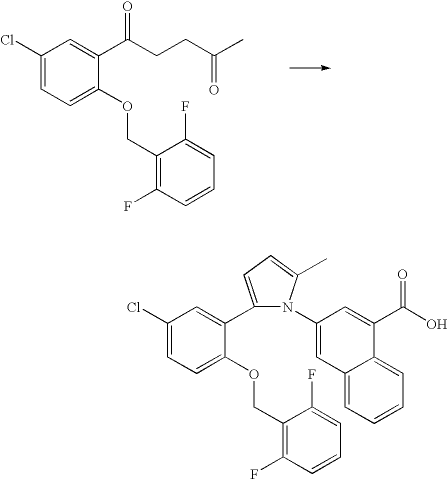

1-[5-Chloro-2-(2,6difluoro-benzyloxy)-phenyl]-pentane-1,4-dione (150 mg, 0.43 mmol), 3-amino-1-naphthoic acid (80 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (66 mg, 31%).

¹H NMR (400 MHz, CDCl₃) 2.18 (3H, s), 4.79 (2H, s), 6.12 (1H, d, J=3.5 Hz), 6.30 (1H, d, J=3.3 Hz), 6.75 (1H, d, J=8.8 Hz), 6.88 (2H, t, J=7.8 Hz), 7.08 (1H, dd, J=2.5 Hz, J=8.8 Hz), 7.23 (1H, d, J=2.5 Hz), 7.24-7.34 (1H, m's excess), 7.54 (1H, t, J=7.3 Hz), 7.62-7.69 (2H, m), 7.73 (1H, d, J=8.1 Hz), 8.12 (1H, d, J=2.3 Hz), 9.07 (1H, d, J=8.6 Hz).

### Example 282

### 3-{2-[5-Chloro-2-(2,6-difluoro-benzyloxy)-phenyl]-5-methyl-pyrrol-1-yl}-4-fluoro-benzoic acid

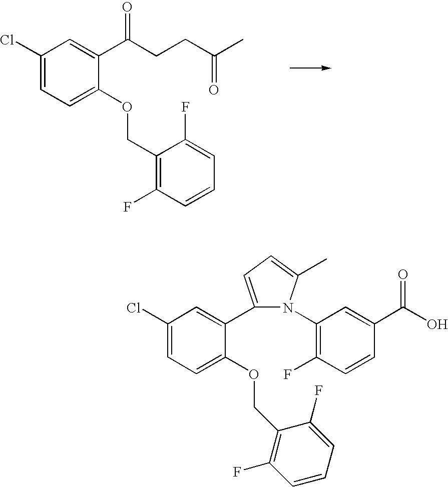

1-[5-Chloro-2-(2,6-difluoro-benzyloxy)-phenyl]-pentane-1,4-dione (150 mg, 0.43 mmol), 3-amino-4-fluorobenzoic acid (66 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (56 mg, 28%).

¹H NMR (400 MHz, CDCl₃) 2.08 (3H, s), 4.85 (2H, s), 6.10 (1H, s), 6.30 (1H, d, J=2.5 Hz), 6.78 (1H, d, J=9.0 Hz), 6.89 (2H, t, J=7.5 Hz), 7.08-7.17 (3H, m), 7.27-7.34 (1H, m's excess), 7.74 (1H, d, J=6.8 Hz), 7.99-8.04 (1H, m).

### Example 283

### 3-{2-[5-Chloro-2-(2,6-difluoro-benzyloxy)-phenyl]-5-methyl-pyrrol-1-yl}-6-methyl-benzoic acid

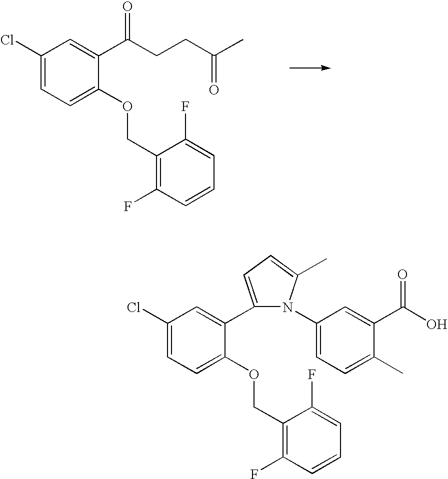

1-[5-Chloro-2-(2,6-difluoro-benzyloxy)-phenyl]-pentane-1,4-dione (131 mg, 0.37 mmol), 3-amino-6-methylbenzoic acid (56 mg, 0.37 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (130 mg, 75%).

¹H NMR (400 MHz, CDCl₃) 2.13 (3H, s), 2.63 (3H, s), 4.82 (2H, s), 6.06 (1H, dd, J=0.8 Hz, J=3.5 Hz), 6.27 (1H, d, J=3.5 Hz), 6.80-6.84 (1H, m), 6.86-6.91 (2H, m), 6.96-7.05 (1H, m), 7.08-7.15 (3H, m), 7.26-7.33 (1H, m's excess), 7.75 (1H, d, J=2.3 Hz).

### Example 284

### 3-{2-[5-Chloro-2-(2,6-difluoro-benzyloxy)-phenyl]-5-methyl-pyrrol-1-yl}-5-bromo-benzoic acid

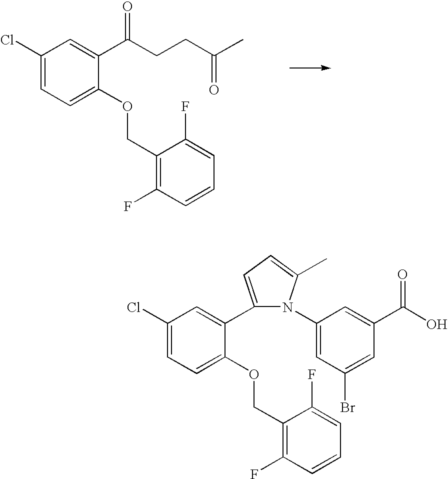

1-[5-Chloro-2-(2,6-difluoro-benzyloxy)-phenyl]-pentane-1,4-dione (131 mg, 0.37 mmol), 3-amino-5-bromobenzoic acid (80 mg, 0.37 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (21 mg, 11%).

¹H NMR (400 MHz, MeOD) 2.07 (3H, s), 4.81 (2H, s), 6.00 (1H, d, J=2.8 Hz), 6.12 (1H, d, J=3.5 Hz), 6.89-6.99 (3H, m), 7.12-7.21 (2H, m), 7.23 (1H, s), 7.32-7.42 (1H, m), 7.52 (1H, s), 7.97 (1H, s).

### Example 285

### 3-{2-[5-Chloro-2-(2,6-difluoro-benzyloxy)-phenyl]-5-methyl-pyrrol-1-yl}-5-amino-benzoic acid

### a) 3-{2-[5-Chloro-2-(2,6-difluoro-benzyloxy)-phenyl]-5-methyl-pyrrol-1-yl}-5-amino-benzoic acid methyl ester

1-[5-Chloro-2-(2,6-difluoro-benzyloxy)-phenyl]-pentane-1,4-dione (150 mg, 0.43 mmol), 3,5-diaminobenzoic acid methyl ester (71 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (97 mg, 48%).

¹H NMR (400 MHz, CDCl₃) 2.12 (3H, s), 3.72 (2H, bs), 3.87 (3H, s), 4.90 (2H, s), 6.03 (1H, d, J=2.8 Hz), 6.30 (1H, d, J=3.5 Hz), 6.45 (1H, s), 6.83-6.95 (3H, m), 7.01-7.11 (2H, m), 7.16 (1H, s), 7.21-7.31 (2H, m's excess).

### b) 3-{2-[5-Chloro-2-(2,6-difluoro-benzyloxy)-phenyl]-5-methyl-pyrrol-1-yl}-5-amino-benzoic acid

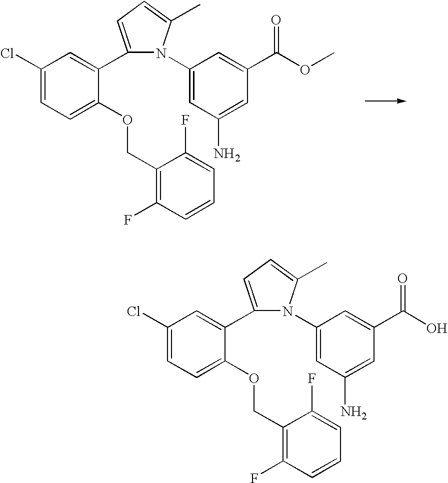

3-{2-[5-Chloro-2-(2,6-difluoro-benzyloxy)-phenyl]-5-methyl-pyrrol-1-yl}-5-amino-benzoic acid methyl ester (97 mg, 0.2 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (72 mg, 77%).

¹H NMR (400 MHz, CDCl₃) 2.17 (3H, s), 4.80 (2H, s), 6.11 (1H, s), 6.28 (1H, s), 6.47-6.55 (1H, m), 6.62 (1H, d, J=7.8 Hz), 6.91-6.99 (1H, m), 7.03-7.11 (1H, m), 7.17-7.34 (5H, m's excess).

LC/MS t=3.55 min, [MH+] 469 and 471, [MH−] 467 and 469.

### Example 286

### 3-{2-[5-Chloro-2-(2,6-difluoro-benzyloxy)-phenyl]-5-methyl-pyrrol-1-yl}-6-acetylamino-benzoic acid

### a) 3-{2-[5-Chloro-2-(2,6-difluoro-benzyloxy)-phenyl]-5-methyl-pyrrol-1-yl}-6-acetylamino-benzoic acid methyl ester

1-[5-Chloro-2-(2,6-difluoro-benzyloxy)-phenyl]-pentane-1,4-dione (150 mg, 0.43 mmol), 3-amino-6-acetylaminobenzoic acid methyl ester (89 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (102 mg, 45%).

¹H NMR (400 MHz, CDCl₃) 2.10 (3H, s), 2.24 (3H, s), 3.86 (3H, s), 4.83 (2H, s), 6.05 (1H, d, J=2.5 Hz), 6.24 (1H, d, J=3.5 Hz), 6.81 (1H, d, J=8.8 Hz), 6.86-6.92 (2H, m), 7.08-7.16 (3H, m), 7.28-7.34 (1H, m), 7.68 (1H, d, J=2.8 Hz), 8.61 (1H, d, J=9.0 Hz), 11.03 (1H, bs).

### b) 3-{2-[5-Chloro-2-(2,6-difluoro-benzyloxy)-phenyl]-5-methyl-pyrrol-1-yl}-6-acetylamino-benzoic acid

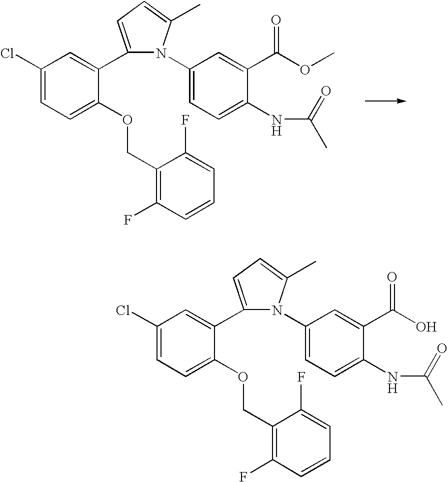

3-{2-[5-Chloro-2-(2,6-difluoro-benzyloxy)-phenyl]-5-methyl-pyrrol-1-yl}-6-acetylamino-benzoic acid methyl ester (102 mg, 0.195 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (78 mg, 78%).

¹H NMR (400 MHz, CDCl₃) 2.11 (3H, s), 2.25 (3H, s), 4.83 (2H, s), 6.05 (1H, d, J=4.3 Hz), 6.24 (1H, d, J=3.5 Hz), 6.82 (1H, d, J=8.5 Hz), 6.86-6.93 (2H, m), 7.06-7.20 (3H, m), 7.27-7.34 (1H, m), 7.74 (1H, d, J=2.8 Hz), 8.63 (1H, d, J=8.8 Hz), 10.88 (1H, bs).

LC/MS t=3.97 min, [MH+] 511 and 513, [MH−] 509 and 511.

### Example 287

### 3-{2-[5-Chloro-2-(2,6-difluoro-benzyloxy)-phenyl]-5-methyl-pyrrol-1-yl}-5-trifluoromethyl-benzoic acid

a) 3-{2-[5-Chloro-2-(2,6-difluoro-benzyloxy)-phenyl]-5-trifluoromethyl-benzoic acid methyl ester

1-[5-Chloro-2-(2,6difluoro-benzyloxy)-phenyl]-pentane-1,4-dione (150 mg, 0.43 mmol), 3-amino-5-trifluoromethylbenzoic acid methyl ester (93 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (0-5%) to give the title compound (73 mg, 32%).

¹H NMR (400 MHz, CDCl₃) 2.15 (3H, s), 3.93 (3H, s), 4.78 (2H, s), 6.09 (1H, d, J=3.5 Hz), 6.25 (1H, d, J=3.5 Hz), 6.81 (1H, d, J=8.5 Hz), 6.83-6.91 (2H, m), 7.12-7.18 (2H, m), 7.26-7.34 (1H, m's excess), 7.39 (1H, s), 7.86 (1H, s), 8.15 (1H, s).

### b) 3-{2-[5-Chloro-2-(2,6-difluoro-benzyloxy)-phenyl]-5-methyl-pyrrol-1-yl}-5-trifluoromethyl-benzoic acid

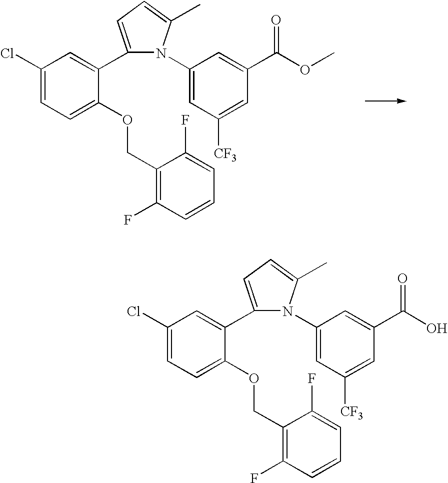

3-{2-[5-Chloro-2-(2,6-difluoro-benzyloxy)-phenyl]-5-methyl-pyrrol-1-yl}-5-trifluoromethyl-benzoic acid methyl ester (73 mg, 0.136 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (55 mg, 78%).

¹H NMR (400 MHz, CDCl₃) 2.17 (3H, s), 4.77 (2H, s), 6.11 (1H, d, J=4.0 Hz), 6.25 (1H, d, J=3.5 Hz), 6.81 (1H, d, J=8.8 Hz), 6.84-6.90 (2H, m), 7.14-7.18 (1H, m), 7.21 (1H, d, J=2.5 Hz), 7.26-7.31 (1H, m's excess), 7.44 (1H, s), 7.90 (1H, s), 8.21 (1H, s).

### Example 288

### 3-{2-[5-Chloro-2-(2,6-difluoro-benzyloxy)-phenyl]-5-methyl-pyrrol-1-yl}-5-(1,1-dioxo-1I⁶-isothiazolidin-2-yl)-benzoic acid

### a) 3-{2-[5-Chloro-2-(2,6-difluoro-benzyloxy)-phenyl]-5-methyl-pyrrol-1-yl}-5-(1,1-dioxo-1I⁶-isothiazolidin-2-yl)-benzoic acid methyl ester

1-[5-Chloro-2-(2,6-difluoro-benzyloxy)-phenyl]-pentane-1,4-dione (150 mg, 0.43 mmol), 3-amino-5-(1,1-dioxo-1I⁶-isothiazolidin-2-yl)-benzoic acid methyl ester (1 15 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (30-50%) to give the title compound (106 mg, 42%).

¹H NMR (400 MHz, CDCl₃) 2.17 (3H, s), 2.47-2.56 (2H, m), 3.36 (2H, t, J=7.5 Hz), 3.59 (2H, t, J=6.5 Hz), 3.89 (3H, s), 4.84 (2H, s), 6.05 (1H, d, J=3.3 Hz), 6.25 (1H, d, J=3.5 Hz), 6.80-6.89 (3H, m), 7.07-7.13 (3H, m), 7.25-7.32 (1H, m's excess), 7.49 (1H, t, J=1.5 Hz), 7.74-7.76 (1H, m).

### b) 3-{2-[5-Chloro-2-2,6-difluoro-benzyloxy)-phenyl]-5-methyl-pyrrol-1-yl}-5-(1,1-dioxo-1I⁶-isothiazolidin-2-yl)-benzoic acid

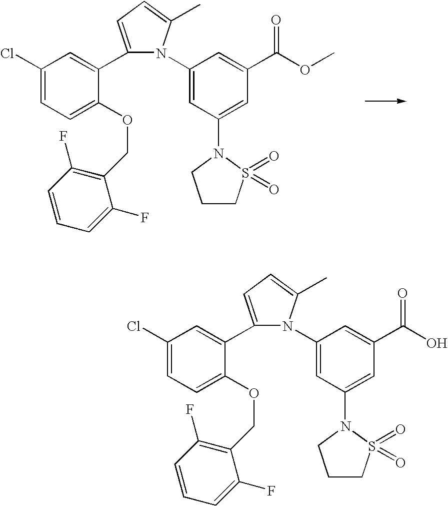

3-{2-[5-Chloro-2-(2,6-difluoro-benzyloxy)-phenyl]-5-methyl-pyrrol-1-yl}-5-(1,1-dioxo-1I⁶-isothiazolidin-2-yl)-benzoic acid methyl ester (106 mg, 0.18 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (71 mg, 69%).

¹H NMR (400 MHz, CDCl₃) 2.19 (3H, s), 2.48-2.57 (2H, m), 3.34-3.40 (2H, m), 3.60 (2H, t, J=6.5 Hz), 4.83 (2H, s), 6.07 (1H, d, J=4.3 Hz), 6.25 (1H, d, J=3.5 Hz), 6.81-6.90 (3H, m), 7.11-7.17 (3H, m), 7.25-7.32 (1H, m's excess), 7.52 (1H, t, J=1.5 Hz), 7.79 (1H, t, J=1.5 Hz).

### Example 289

### 3-{2-[5-Chloro-2-(2,3-difluoro-benzyloxy)-phenyl]-5-methyl-pyrrol-1-yl}-5-hydroxy-benzoic acid

### a) 5-Chloro-2-(2,3-difluoro-benzyloxy)-benzaldehyde

5-Chlorosalicylaldehyde (10g, 63.7 mmol), 2,3-difluorobenzyl bromide (8.08 ml, 63.7 mmol) and K₂CO₃ (17.6g, 127.4 mmol) were heated in DMF (64 ml, 1M) at 60° C. for 3 hrs. Upon cooling to room temperature, Et₂O and H₂O were added. The layers were separated and the aqueous phase was extracted with Et₂O. The combined organic extracts were dried (Na₂SO₄), filtered and concentrated to give the title compound (13.4 g, 74.3%).

¹H NMR (400 MHz, CDCl₃) 5.27 (2H, s), 7.04 (1H, d, J=8.8 Hz), 7.20 (3H, m's excess), 7.50 (1H, dd, J=2.8 Hz, J=9.0 Hz), 7.81 (1H, d, J=2.8 Hz), 10.44 (1H, s).

### b) 1-[5-Chloro-2-(2,3-difluoro-benzyloxy)-phenyl]-pentane-1,4-dione

A mixture of 5-chloro-2-(2,3-difluoro-benzyloxy)-benzaldehyde (13.4g, 47.3 mmol), methyl vinyl ketone (3.36 ml, 56.8 mmol), 3-ethyl-5-(2-hydroxyethyl)-4-methylthiazolium bromide (1.7g, 7.1 mmol, 0.15 eq) and triethylamine (6.07 ml, 82.8 mmol) was heated in EtOH (16 ml, 3M) at 80° C. for 18 hours. Upon cooling, the mixture was diluted with EtOAc and washed with sat. NH₄Cl and sat. NaHCO₃, dried (Na₂SO₄) filtered and concentrated. The residue was purified by chromatography, using Biotage®, with cyclohexane containing a gradient of EtOAc (10-15%) to give the title compound (4.16g, 25%).

¹H NMR (400 MHz, CDCl₃) 2.21 (3H, s), 2.81 (2H, t, J=6.3 Hz), 3.21 (2H, t, J=6.3 Hz), 5.24 (2H, s), 6.99 (1H, d, J=9.0 Hz), 7.10-7.31 (3H, m's excess), 7.41 (1H, dd, J=2.8 Hz, J=8.8 Hz), 7.71 (1H, d, J=2.8 Hz).

### c) 3-{2-[5-Chloro-2-(2,3-difluoro-benzyloxy)-phenyl]-5-methyl-pyrrol-1-yl}-5-hydroxy-benzoic acid methyl ester

1-[5-Chloro-2-(2,3-difluoro-benzyloxy)-phenyl]-pentane-1,4-dione (150 mg, 0.42 mmol), 3-amino-5-hydroxybenzoic acid methyl ester (71 mg, 0.42 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (91 mg, 45%).

¹H NMR (400 MHz, CDCl₃) 2.16 (3H, s), 3.84 (3H, s), 4.84 (2H, s), 5.19 (1H, s), 6.12 (1H, d, J=2.8 Hz), 6.29 (1H, d, J=3.5 Hz), 6.61-6.67 (2H, m), 6.80-6.87 (1H, m), 6.97-7.04 (1H, m), 7.05-7.13 (1H, m), 7.20-7.24 (1H, m), 7.30 (1H, t, J=1.5 Hz), 7.41 (1H, m).

### d) 3-{2-[5-Chloro-2-(2,3-difluoro-benzyloxy)-phenyl]-5-methyl-pyrrol-1-yl}-5-hydroxy-benzoic acid

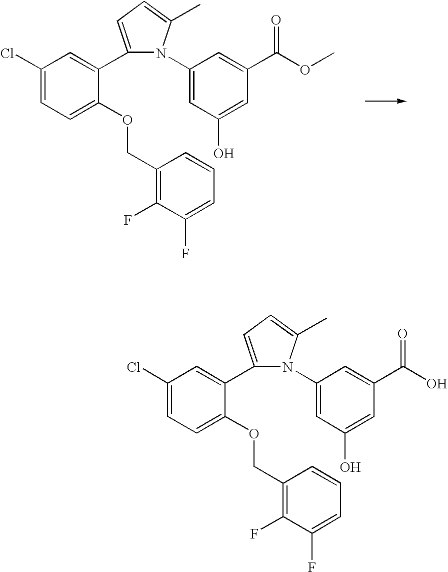

3-{2-[5-Chloro-2-(2,3-difluoro-benzyloxy)-phenyl]-5-methyl-pyrrol-1-yl}-5-hydroxy-benzoic acid methyl ester (91 mg, 0.19 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (78 mg, 87%).

¹H NMR (400 MHz, MeOD) 2.11 (3H, s), 4.86 (2H, m's excess), 6.19 (1H, s), 6.57 (1H, t, J=2.3 Hz), 6.81 (1H, d, J=8.8 Hz), 6.85-6.91(1H, m), 7.02-7.21 (6H, m), 7.30 (1H, s).

LC/MS t=3.74 min [MH+] 470, [MH−] 468

### Example 290

### 3-{2-[5-Chloro-2-(2,3-difluoro-benzyloxy)-phenyl]-5-methyl-pyrrol-1-yl}-6-chloro-benzoic acid

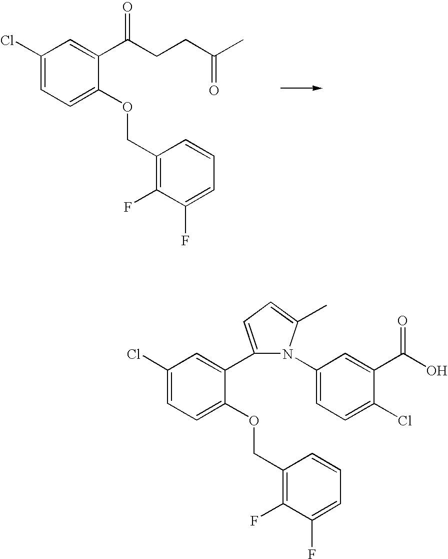

1-[5-Chloro-2-(2,3-difluoro-benzyloxy)-phenyl]-pentane-1,4-dione (150 mg, 0.43 mmol), 3-amino-6-chlorobenzoic acid (73.3 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (109 mg, 53%).

¹H NMR (400 MHz, CDCl₃) 2.17 (3H, s), 4.84 (2H, s), 6.14 (1H, d, J=3.3 Hz), 6.28 (1H, d, J=3.3 Hz), 6.61-6.72 (2H, m), 6.97-7.16 (5H, m), 7.28-7.36 (1H, m's excess), 7.69 (1H, d, J=2.5 Hz).

LC/MS t=4.10 min [MH+] 489.

### Example 291

### 3-{2-[5-Chloro-2-(2,3-difluoro-benzyloxy)-phenyl]-5-methyl-pyrrol-1-yl}-6-fluoro-benzoic acid

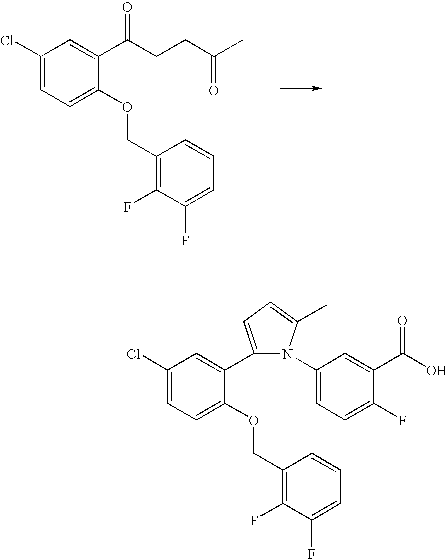

1-[5-Chloro-2-(2,3difluoro-benzyloxy)-phenyl]-pentane-1,4-dione (150 mg, 0.43 mmol), 3-amino-6-fluorobenzoic acid (66 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (61 mg, 31%).

¹H NMR (400 MHz, CDCl₃) 2.16 (3H, s), 4.86 (2H, s), 6.15 (1H, d, J=3.3 Hz), 6.29 (1H, d, J=3.5 Hz), 6.65 (1H, d, J=8.8 Hz), 6.78 (1H, t, J=6.0 Hz), 6.98-7.19 (6H, m), 7.71 (1H, dd, J=2.8 Hz, J=6.3 Hz).

LC/MS t=3.92 min [MH+] 472, [MH−] 470.

### Example 292

### 3-{2-[5-Chloro-2-(2,3-difluoro-benzyloxy)-phenyl]-5-methyl-pyrrol-1-yl}-5-acetylamino-benzoic acid

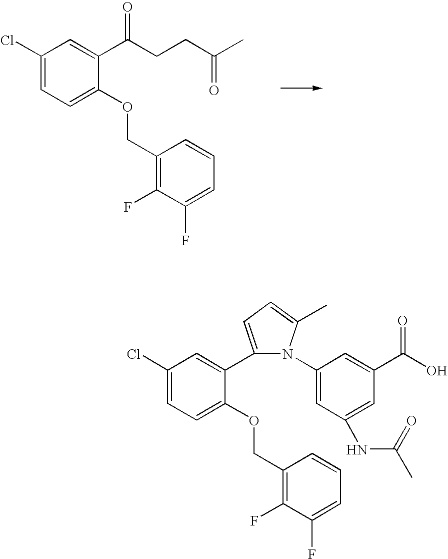

1-[5-Chloro-2-(2,3-difluoro-benzyloxy)-phenyl]-pentane-1,4-dione (150 mg, 0.43 mmol), 3-amino-5-acetylaminobenzoic acid (83 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (30-50%) to give the title compound (44 mg, 21%).

¹H NMR (400 MHz, MeOD) 2.05 (3H, s), 2.11 (3H, s), 4.84 (2H, s), 6.03 (1H, d, J=3.0 Hz), 6.20 (1H, d, J=3.5 Hz), 6.73 (1H, d, J=8.8 Hz), 6.85 (1H, t, J=7.3 Hz), 7.00-7.09 (2H, m), 7.10-7.21 (2H, m), 7.35 (1H, s), 7.50 (1H, s), 7.98 (1H, s).

LC/MS t=3.60 min [MH+] 511

### Example 293

### 3-{2-[5-Chloro-2-(2,3-difluoro-benzyloxy)-phenyl]-5-methyl-pyrrol-1-yl}-1-naphthoic acid

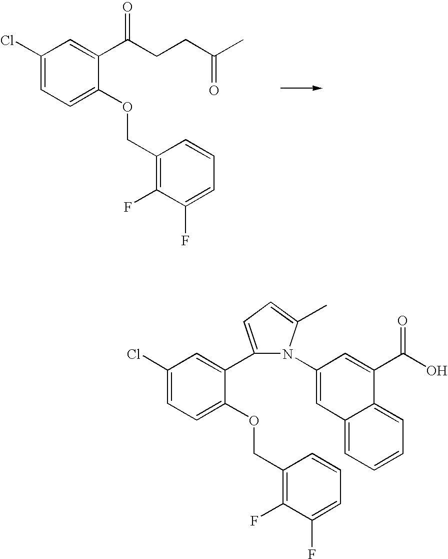

1-[5-Chloro-2-(2,3-difluoro-benzyloxy)-phenyl]-pentane-1,4-dione (150 mg, 0.43 mmol), 3-amino-1-naphthoic acid (80 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-15%) to give the title compound (59 mg, 28%).

¹H NMR (400 MHz, CDCl₃) 2.22 (3H, s), 4.77 (2H, s), 6.20 (1H, d, J=2.8 Hz), 6.34 (1H, d, J=3.5 Hz), 6.47-6.60 (2H, m), 6.80 (1H, q, J=8.1 Hz), 6.97-7.09 (2H, m), 7.39 (1H, d, J=2.8 Hz), 7.54 (1H, t, J=7.3 Hz), 7.61-7.71 (3H, m), 8.13 (1H, s), 9.05 (1H, d, J=9.1 Hz).

### Example 294

### 3-{2-[5-Chloro-2-(2,3-difluoro-benzyloxy)-phenyl]-5-methyl-pyrrol-1-yl}-4-fluoro-benzoic acid

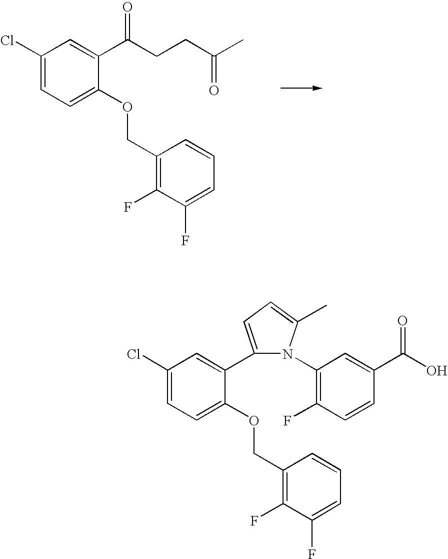

1-[5-Chloro-2-(2,3-difluoro-benzyloxy)-phenyl]-pentane-1,4-dione (150 mg, 0.43 mmol), 3-amino-4-fluorobenzoic acid (66 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-15%) to give the title compound (48 mg, 24%).

¹H NMR (400 MHz, CDCl₃) 2.12 (3H, s), 4.90 (2H, s), 6.17 (1H, d, J=2.8 Hz), 6.33 (1H, d, J=3.3 Hz), 6.62 (1H, d, J=8.8 Hz), 6.82 (1H, t, J=6.3 Hz), 6.94-7.20 (4H, m), 7.24 (1H, d, J=2.5 Hz), 7.80 (1H, dd, J=2.3 Hz, J=7.1 Hz), 8.01-8.07 (1H, m).

### Example 295

### 3-{2-[5-Chloro-2-(2,3-difluoro-benzyloxy)-phenyl]-5-methyl-pyrrol-1-yl}-6-methyl-benzoic acid

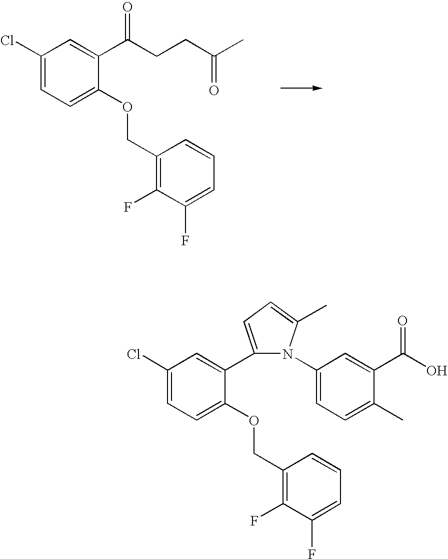

1-[5-Chloro-2-(2,3-difluoro-benzyloxy)-phenyl]-pentane-1,4-dione (1 50 mg, 0.43 mmol), 3-amino-6-methylbenzoic acid (64.3 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-15%) to give the title compound (1 16 mg, 58%).

¹H NMR (400 MHz, CDCl₃) 2.17 (3H, s), 2.64 (3H, s), 4.84 (2H, s), 6.14 (1H, d, J=3.3 Hz), 6.30 (1H, d, J=3.3 Hz), 6.62 (1H, d, J=8.8 Hz), 6.74-6.80 (1H, m), 6.95-7.16 (6H, m), 7.78 (1H, d, J=2.0 Hz).

### Example 296

### 3-{2-[5-Chloro-2-(2,3-difluoro-benzyloxy)-phenyl]-5-methyl-pyrrol-1-yl}-5-bromo-benzoic acid

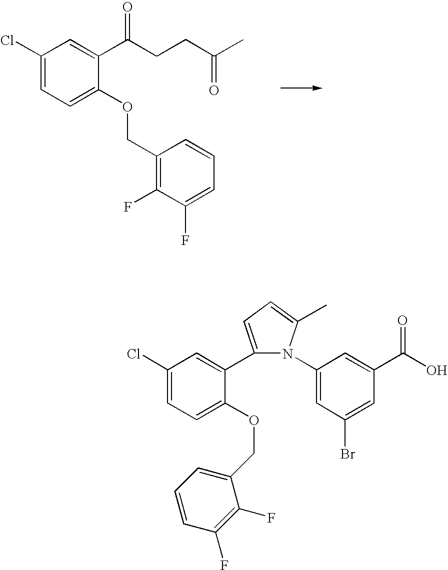

1-[5-Chloro-2-(2,3difluoro-benzyloxy)-phenyl]-pentane-1,4-dione (150 mg, 0.43 mmol), 3-amino-5-bromobenzoic acid (92 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-20%) to give the title compound (48 mg, 21%).

¹H NMR (400 MHz, CDCl₃) 2.17 (3H, s), 4.82 (2H, s), 6.14 (1H, d, J=2.5 Hz), 6.30 (1H, d, J=3.5 Hz), 6.66 (1H, d, J=8.8 Hz), 6.75-6.82 (1H, m), 6.98-7.18 (4H, m), 7.34 (1H, s), 7.65 (1H, s), 8.10 (1H, s).

### Example 297

### 3-{2-[5-Chloro-2-(2,3-difluoro-benzyloxy)-phenyl]-5-methyl-pyrrol-1-yl}-5-amino-benzoic acid

### a) 3-{2-[5-Chloro-2-(2,3-difluoro-benzyloxy)-phenyl]-5-methyl-pyrrol-1-yl}-5-amino-benzoic acid methyl ester

1-[5-Chloro-2-(2,3-difluoro-benzyloxy)-phenyl]-pentane-1,4-dione (150 mg, 0.43 mmol), 3,5-diaminobenzoic acid methyl ester (71 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-15%) to give the title compound (102 mg, 50%).

¹H NMR (400 MHz, CDCl₃) 2.15 (3H, s), 3.82 (3H, s), 4.86 (2H, s), 6.10 (1H, d, J=2.8 Hz), 6.30 (1H, d, J=3.3 Hz), 6.64 (1H, d, J=8.9 Hz), 6.85 (1H, t, J=6.3 Hz), 6.97-7.05 (1H, m), 7.06-7.14 (3H, m), 7.19-7.24 (2H, m).

### b) 3-{2-[5-Chloro-2-(2,3-difluoro-benzyloxy)-phenyl]-5-methyl-pyrrol-1-yl}-5-amino-benzoic acid

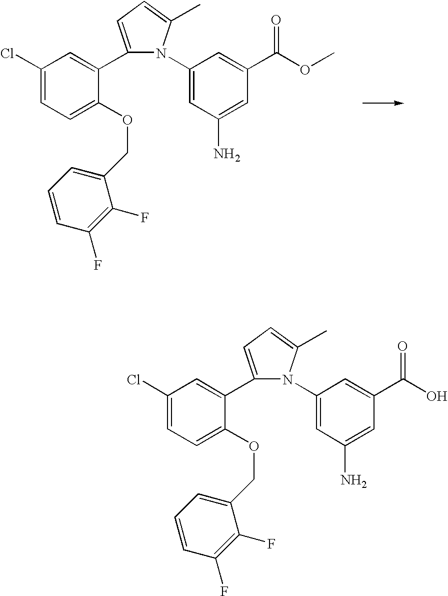

3-{2-[5-Chloro-2-(2,3-difluoro-benzyloxy)-phenyl]-5-methyl-pyrrol-1-yl}-5-amino-benzoic acid methyl ester (102 mg, 0.21 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (70 mg, 71%).

¹H NMR (400 MHz, MeOD) 2.13 (3H, s), 4.84 (2H, s), 6.11 (1H, d, J=2.8 Hz), 6.23 (1H, d, J=3.5 Hz), 6.81-6.91 (2H, m), 7.03-7.12 (1H, m), 7.13-7.27 (4H, m), 7.66 (1H, s), 7.85 (1H, s).

LC/MS t=3.63 min [MH+] 469, [MH−] 467

### Example 298

### 3-{2-[5-Chloro-2-(2,3-difluoro-benzyloxy)-phenyl]-5-methyl-pyrrol-1-yl}-6-acetylamino-benzoic acid

### a) 3-{2-[5-Chloro-2-(2,3-difluoro-benzyloxy)-phenyl]-5-methyl-pyrrol-1-yl}-6-acetylamino-benzoic acid methyl ester

1-[5-Chloro-2-(2,3-difluoro-benzyloxy)-phenyl]-pentane-1,4-dione (150 mg, 0.43 mmol), 3-amino-6-acetylaminobenzoic acid methyl ester (89 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (10-15%) to give the title compound (1 10 mg, 50%).

¹H NMR (400 MHz, MeOD) 2.09 (3H, s), 2.19 (3H, s), 3.79 (3H, s), 4.83 (2H, s), 6.06 (1H, d, J=3.5 Hz), 6.18 (1H, d, J=3.5 Hz), 6.81 (1H, d, J=8.8 Hz), 6.85-6.91 (1H, m), 7.03-7.27 (5H, m), 7.58 (1H, d, J=2.5 Hz), 8.34 (1H, d, J=8.8 Hz).

### b) 3-{2-[5-Chloro-2-(2,3-difluoro-benzyloxy)-phenyl]-5-methyl-pyrrol-1-yl}-acetylamino-benzoic acid

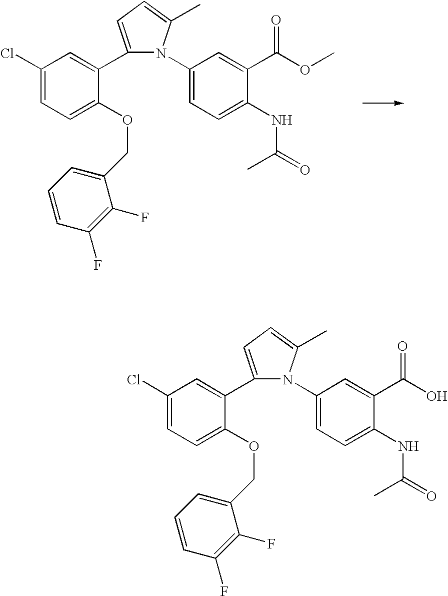 b 3-{2-[5-Chloro-2-(2,3-difluoro-benzyloxy)-phenyl]-5-methyl-pyrrol-1-yl}-6-acetylamino-benzoic acid methyl ester (1 0 mg, 0.21 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (86 mg, 80%).

¹H NMR (400 MHz, MeOD) 2.08 (3H, s), 2.17 (3H, s), 4.83 (2H, s), 6.80 (1H, d, J=8.8 Hz), 6.82-6.89 (1H, m), 7.00-7.25 (7H, m), 7.64 (1H, d, J=2.8 Hz), 8.41 (1H, d, J=9.0 Hz).

LC/MS t=511 min [MH+] 511, [MH−] 509.

### Example 299

### 3-(2-[5-Chloro-2-(2,3-difluoro-benzyloxy)-phenyl]-5-methyl-pyrrol-1-yl}-5-trifluoromethyl-benzoic acid

### a) 3-{2-[5-Chloro-2-(2,3-difluoro-benzyloxy)-phenyl]-5-methyl-pyrrol-1-yl}-5-trifluoromethyl-benzoic acid methyl ester

1-[5-Chloro-2-(2,3-difluoro-benzyloxy)-phenyl]-pentane-1,4-dione (150 mg, 0.43 mmol), 3-amino-5trifluoromethylbenzoic acid methyl ester (93 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (0-5%) to give the title compound (1 07 mg, 48%).

¹H NMR (400 MHz, CDCl₃) 2.17 (3H, s), 3.90 (3H, s), 4.77 (2H, s), 6.16 (1H, d, J=3.0 Hz), 6.31 (1H, d, J=3.5 Hz), 6.62 (1H, d, J=8.8 Hz), 6.71-6.77 (1H, m), 6.96-7.03 (1H, m), 7.06-7.16 (2H, m), 7.25-7.31 (1H, m's excess), 7.39 (1H, s), 7.84 (1H, s), 8.15 (1H, s).

### b) 3-{2-[5-Chloro-2-(2,3-difluoro-benzyloxy)-phenyl]-5-methyl-pyrrol-1-yl}-5-trifluoromethyl-benzoic acid

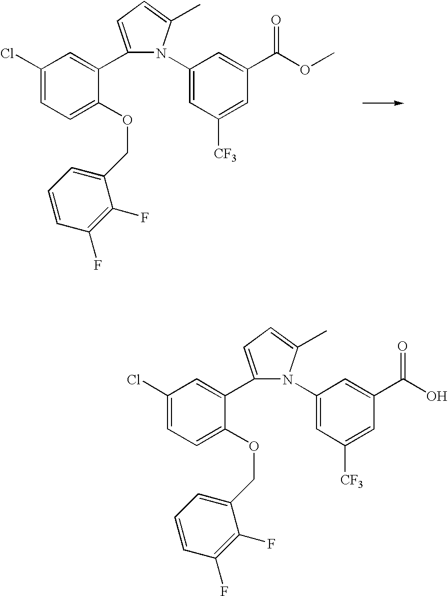

3-{2-[5-Chloro-2-(2,3-difluoro-benzyloxy)-phenyl]-5-methyl-pyrrol-1-yl}-5-trifluoromethyl-benzoic acid methyl ester (1 07 mg, 0.20 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (73 mg, 70%).

¹H NMR (400 MHz, MeOD) 2.14 (3H, s), 4.76 (2H, s), 6.12 (1H, d, J=2.8 Hz), 6.23 (1H, d, J=3.5 Hz), 6.77-6.86 (2H, m), 7.02-7.11 (1H, m), 7.14-7.23 (2H, m), 7.29 (1H, d, J=2.8 Hz), 7.37 (1H, s), 7.78 (1H, s), 8.09 (1H, s).

### Example 300

### 3-{2-[5-Chloro-2-(2,3difluoro-benzyloxy)-phenyl]-5-methyl-pyrrol-1-yl}-5-(1,1-dioxo-1I⁶-isothiazolidin-2-yl)-benzoic acid

### a) 3-{2-[5-Chloro-2-(2,3-difluoro-benzyloxy)-phenyl]-5-methyl-pyrrol-1-yl}-5-(1,1-dioxo-1I⁶ isothiazolidin-2-yl)-benzoic acid methyl ester

1-[5-Chloro-2-(2,3-difluoro-benzyloxy)-phenyl]-pentane-1,4-dione (150 mg, 0.43 mmol), 3-amino-5-(1,1-dioxo-1I⁶-isothiazolidin-2-yl)-benzoic acid methyl ester (1 15 mg, 0.43 mmol) and pTSA (5 mg) were heated in acetonitrile (2 ml) at 160° C. for 10 minutes in the microwave. Upon cooling, the mixture was diluted with DCM and washed with 2M HCl, dried (Na₂SO₄), filtered and concentrated. The residue was purified by chromatography, on silica, with cyclohexane containing a gradient of EtOAc (20-30%) to give the title compound (152 mg, 62%).

¹H NMR (400 MHz, MeOD) 2.14 (3H, s), 2.44 (2H, quin, J=6.8 Hz), 3.39 (2H, t, J=7.3 Hz), 3.55 (2H, t, J=6.5 Hz), 3.82 (3H, s), 4.81 (2H, s), 6.08 (1H, d, J=3.3 Hz), 6.20 (1H, d, J=3.5 Hz), 6.76-6.85 (2H, m), 7.02-7.10 (2H, m), 7.12-7.20 (2H, m), 7.24 (1H, d, J=2.5 Hz), 7.29 (1H, t, J=1.5 Hz), 7.71 (1H, m).

### b) 3-{2-[5-Chloro-2-(2,3-difluoro-benzyloxy)-phenyl]-5ethyl-pyrrol-1-yl}-5-(1,1-dioxo-1I⁶ isothiazolidin-2-yl)-benzoic acid

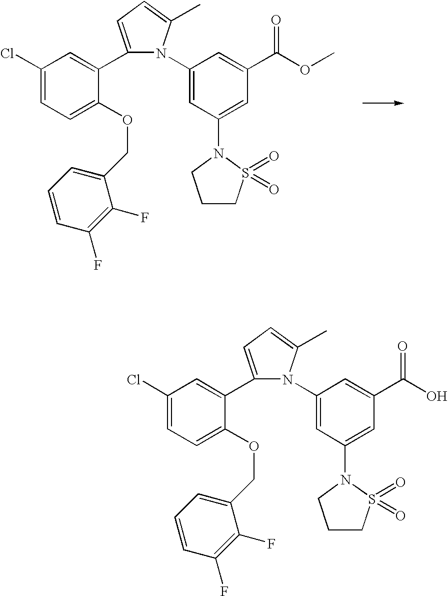

3-{2-[5-Chloro-2-(2,3-difluoro-benzyloxy)-phenyl]-5-methyl-pyrrol-1-yl}-5-(1,1-dioxo-1I⁶-isothiazolidin-2-yl)-benzoic acid methyl ester (152 mg, 0.26 mmol) was heated in a mixture of EtOH (4 ml) and 2M NaOH (0.5 ml) at 120° C. for 5 minutes in the microwave. Upon cooling, the mixture was diluted with DCM, washed with 2M HCl, dried (Na₂SO₄), filtered and evaporated to give the title compound (1 20 mg, 95%).

¹H NMR (400 MHz, MeOD) 2.14 (3H, s), 2.44 (2H, quin, J=7.0 Hz), 3.38 (2H, t, J=7.3 Hz), 3.52-3.64 (2H, m's excess), 4.83 (2H, s), 6.19 (1H, s), 6.75-6.91 (2H, m), 6.99-7.28 (6H, m), 7.32 (1H, t, J=1.5 Hz), 7.72 (1H, m).

### Example 301

### 3-{2-[5-Bromo-2-(2,4-difluoro-benzyloxy)-phenyl]-5-methyl-pyrrol-1-yl}-5-propionylamino-benzoic acid

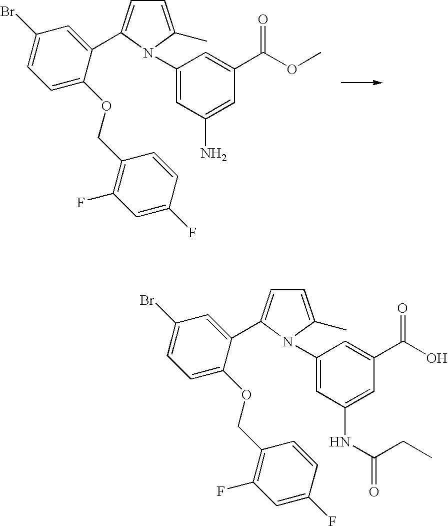

5-{2-[5-Bromo-2-(2,4difluoro-benzyloxy)-phenyl]-5-methyl-pyrrol-1-yl}-5-amino-benzoic acid methyl ester (264 mg, 0.5 mmol) was dissolved DCM (2 mL) containing pyridine (0.1 mL) and DMAP (60 mg, cat). Propionyl chloride (70 μL) was added dropwise. The reaction mixture was stirred overnight at room temperature, then diluted with DCM (15 mL) and washed sequentially with 2M HCl and water, dried (MgSO₄), filtered and evaporated. The residue was purified on a Water's sep-pack (10.0 g) with Et₂O/iso-hexane to give, after evaporation, the intermediate ester. The ester was then heated at 60° C. for 1 hour in MeOH (3 mL) and 2M NaOH (2 mL). Upon cooling the solvent volume was reduced to approximately 1 mL then neutralised with 2M HCl (2 mL), diluted with water (1 0 mL) and extracted with DCM (10 mL). The organic extracts were dried (MgSO₄), filtered and concentrated to give the title compound (80 mg, 30%).

¹H NMR (400 MHz, CDCl₃) 1.22 (3H, t ,J=7 Hz), 2.17 (3H, s), 2.33-2.42 (2H, m), 4.78 (2H, s), 6.11 (1H, d, J=3 Hz), 6.27 (1H, d, J=3 Hz), 6.56 (1H, d J=9 Hz), 6.72-6.83 (2H, m), 6.99-7.07 (1H, m), 7.17-7.22 (2H, m), 7.35 (1H, d, J=2 Hz), 7.45 (1H, s), 7.62 (1H, s), 7.98 (1H, s).

LC/MS t=3.88 min [MH⁺] 571

### Example 302

### 3-{2-[5-Bromo-2-(2,4-difluoro-benzyloxy)-phenyl]-5-methyl-pyrrol-1-yl}-5-butyrylamino-benzoic acid

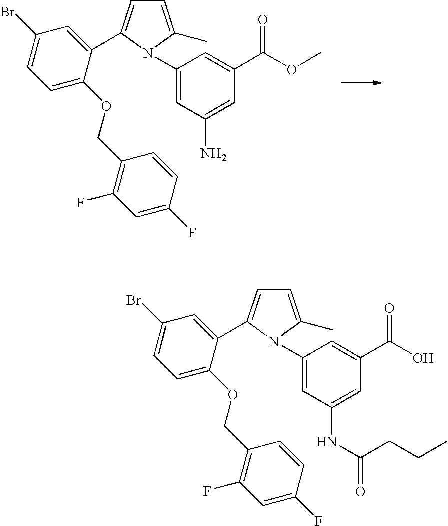

Prepared in the same way as 3-{2-[5-Bromo-2-(2,4-difluoro-benzyloxy)-phenyl]-5-methyl-pyrrol-1-yl}-5-propionylamino-benzoic acid using the appropriate acid chloride, to give the title compound (80 mg, 28%).

¹H NMR (400 MHz, CDCl₃) 0.98 (3H, t, J=7 Hz), 1.67-1.78 (2H, m), 2.16 (3H, s), 2.26-2.34 (2H, m), 4.78 (2H, s), 6.11 (1H, d, J=3 Hz), 6.27 (1H, d, J=3 Hz), 6.56 (1H, d, J=9 Hz), 6.72-6.83 (2H, m), 6.96-7.06 (1H, m), 7.17-7.23 (2H, m), 7.36 (1H, d, J=2 Hz), 7.46 (1H, s), 7.61 (1H, s), 7.98 (1H, s)

LC/MS t=3.98 min [MH⁺] 585.

### Example 303

### 3-{2-[5-Bromo-2-(2,4-difluoro-benzyloxy)-phenyl]-5-methyl-pyrrol-1-yl}-5-(2-methoxy-ethanolamino)-benzoic acid

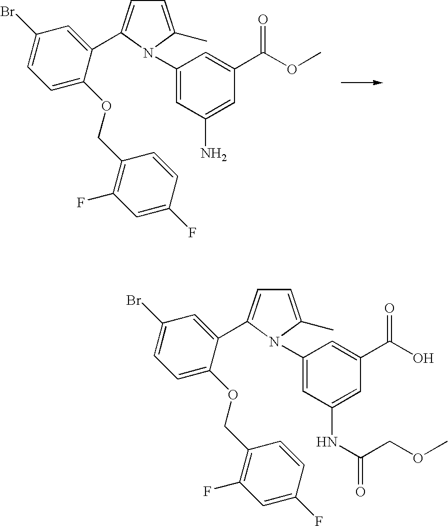

Prepared in the same way as 3-{2-[5-Bromo-2-(2,4-difluoro-benzyloxy)-phenyl]-5-methyl-pyrrol-1-yl}-5-propionylamino-benzoic acid using the appropriate acid chloride, to give the title compound (60 mg, 20%).

¹H NMR (400 MHz, CDCl₃) 2.16 (3H, s), 3.50 (3H, s), 3.99 (2H, s), 4.79 (2H, s), 6.11 (1H, d, J=3 Hz), 6.28 (1H, d, J=3 Hz), 6.57 (1H, d, J=9 Hz), 6.73-6.84 (2H, m), 6.98 7.08 (1H, m), 7.22 (1H, d, J=2 Hz), 7.37 (1H, d, J=2 Hz), 7.47 (1H, t, J=1.5 Hz), 7.73 (1H, t, J=1.5 Hz), 7.99 (1H, t, J=1.5 Hz), 8.26 (1H,s).

LC/MS t=3.81 min [MH⁺] 587

### Example 304

### 3-{2-[5-Bromo-2-(2,4-difluoro-benzyloxy)-phenyl]-5-methyl-pyrrol-1-yl}-5-(2-thiophen-2-yl-ethanoylamino)-benzoic acid

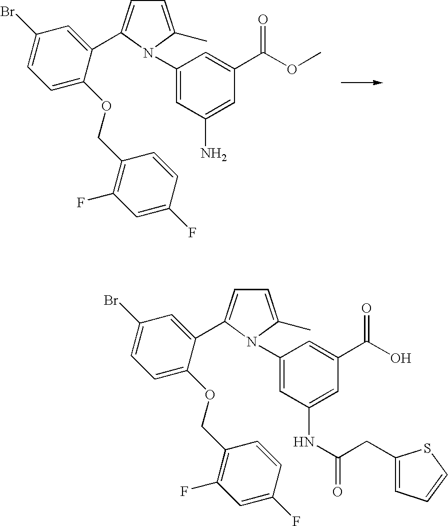

Prepared in the same way as 3-{2-[5-Bromo-2-(2,4-difluoro-benzyloxy)-phenyl]-5-methyl-pyrrol-1-yl}-5-propionylamino-benzoic acid using the appropriate acid chloride, to give the title compound (120 mg, 38%).

¹H NMR (400 MHz, CDCl₃) 2.16 (3H, s), 3.92 (2H, s), 4.77 (2H, s), 6.10 (1H, d, J=3 Hz), 6.26 (1H, d, J=3 Hz), 6.55 (1H, d, J=8 Hz), 6.72-6.82 (2H, m), 6.96-7.18 (3H,m),7.17-7.24 (1H, r), 7.29-7.36 (3H, m), 7.46 (1H, t, J=1.5 Hz), 7.60 (1H, t, J=1.5 Hz), 7.83 (1H, t, J=1.5 Hz).

LC/MS t=4.06 min [MH+] 639

### Example 305

### 3-{2-[5-Bromo-2-(2,4-difluoro-benzyloxy)-phenyl]-5-methyl-pyrrol-1-yl}-(2-methyl-propanoylamino)-benzoic acid

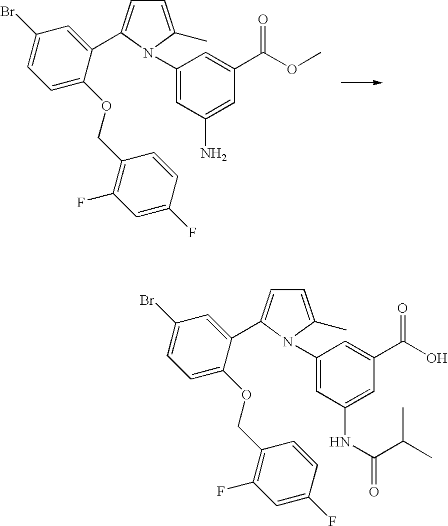

Prepared in the same way as 3-{2-[5-Bromo-2-(2,4-difluoro-benzyloxy)-phenyl]-5-methyl-pyrrol-1-yl-}-5-propionylamino-benzoic acid using the appropriate acid chloride, to give the title compound (170 mg, 58%).

¹H NMR (400 MHz, CDCl₃) 1.23 (6H, d, J=7 Hz), 2.17 (3H, s), 2.43-2.52 (1H, m), 4.79 (2H, s), 6.10 (1H, d, J=3 Hz), 6.27 (1H, d, J=3 Hz), 6.57 (1H, d, J=9 Hz), 6.72-6.83 (2H, m), 6.98 (1H, m), 7.17 (2H, m), 7.36 (1H, d, J=3 Hz), 7.40 (1H, br s), 7.56 (1H, br s), 7.99 (1H, br s).

LC/MS t=3.99 min [MH⁺] 585.

### Example 306

### 3-{2-[5-Bromo-2-(2,4-difluoro-benzyloxy)-phenyl]-5-methyl-pyrrol-1-yl}-5-methanesulfonylamino-benzoic acid

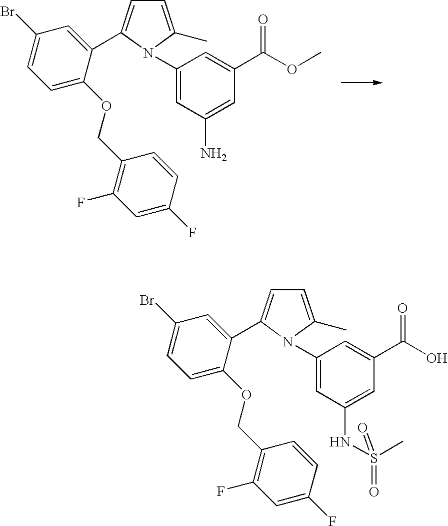

Prepared in the same way as 3-{2-[5-Bromo-2-(2,4difluorobenzyloxy)-phenyl]-5-methyl-pyrrol-1-yl}-5-propionylamino-benzoic acid, to give the title compound (150 mg, 58%).

¹H NMR (400 MHz, CDCl₃) 2.18 (3H, s), 2.72 (3H, s), 4.78 (2H, s), 6.13 (1H, d, J=3 Hz) 6.30 (1H, d, J=3 Hz), 6.62 (1H, d, J=8 Hz), 6.74-6.85 (2H, m), 7.00-7.10 (2H, m), 7.17-7.24 (2H, m), 7.34 (1H, s), 7.57 (1H, s), 7.73 (1H, s).

LC/MS t=3.81 min [MH⁺] 593.

### Example 307

### 3-{2-[5-Bromo-2-(2,4-difluoro-benzyloxy)-phenyl]-5-methyl-pyrrol-1-yl}-5-dimethylamino-benzoic acid

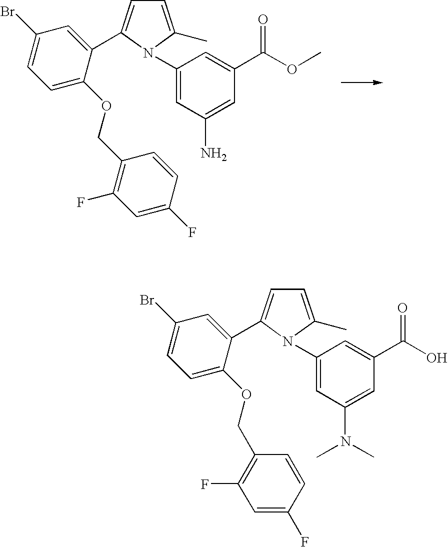

3-{2-[5-Bromo-2-(2,4-difluoro-benzyloxy)-phenyl]-5-methyl-pyrrol-1-yl}-5amino-benzoic acid methyl ester (236 mg, 0.5 mmol) in DMF (2.5 mL), was treated with sodium hydride (60% suspension) (100 mg, 2.5 mmol) under nitrogen. The reaction mixture was stirred for 30 minutes at room temperature before adding iodomethane (0.2 ml, 1.3 mmol). The mixture was stirred at room temperature for a further 2 hours, then quenched with water (2 mL) and stirred over night. The mixture was then further diluted with water (10 mL) and the pH was adjusted to pH˜6 with glacial acetic acid, extacted with DCM, dried (MgSO₄), filtered and concentrated. The residue was chromatographed on a Water's sep-pack (10 g) with Et₂O/iso-hexane to give the title compound (70 mg, 25%).

¹H NMR (400 MHz, CDCl₃) 2.20 (3H, s), 2.80 (6H, s), 4.77 (2H, s), 6.12 (1H, d, J=3 Hz), 6.29 (1H, d, J=3 Hz), 6.45 (1H, t, J=2 Hz), 6.56 (1H, d, J=9 Hz), 6.72-6.82 (2H, m), 6.94-7.03 (1H, m), 7.15 (1H, s), 7.18-7.24 (1H, m), 7.28 (1H, s), 7.38 (1H, d, J=2 Hz)

LC/MS t=4.10 mins [MH⁺] 543.

### Example 308

### 3-{2-[5-Bromo-2-(2,4-difluoro-benzyloxy)-phenyl]-5-methyl-pyrrol-1-yl}-5-ethylamino-benzoic acid

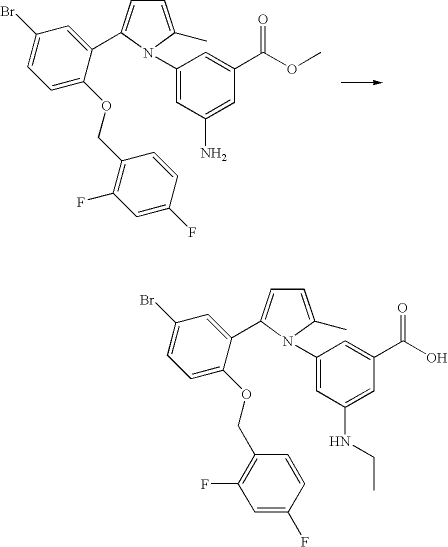

Prepared in the same way as 3-{2-[5-Bromo-2-(2,4difluoro-benzyloxy)-phenyl]-5-methyl-pyrrol-1-yl}-5dimethylamino-benzoic acid, using 2.6 equivalent of ethyl iodide (60 mg, 22%).

¹H NMR (400 MHz, CDCl₃) 1.23 (3H, t, J=7 Hz), 2.18 (3H, s), 2.98 (2H, q, J=7 Hz), 4.80 (2H, s), 6.10 (1H, d, J=3 Hz), 6.28 (1H, d, J=3 Hz), 6.36 (1H, t, J=1.5 Hz), 6.58 (1H, d, J=9 Hz), 6.73-6.83 (2H, m), 6.99-7.06 (2H, m), 7.10 (1H, t, J=1.5 Hz), 7.17 (1H, t, J=1.5 Hz), 7.18-7.23 (1H, dd, J=3 Hz, J=7 Hz), 7.36 (1H, d, J=3 Hz).

LC/MS t=4.02 min [MH⁺] 543.

### Example 309

### 3-{2-[5-Chloro-2-(2,4-difluoro-benzyloxy)-phenyl]-5-methyl-pyrrol-1-yl}-5-ethylamino-benzoic acid

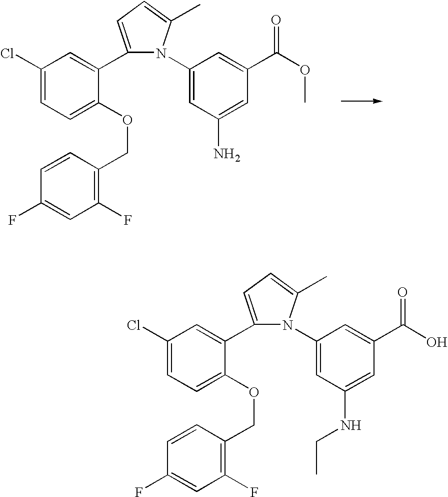

Prepared in the same way as 3-{2-[5-Bromo-2-(2,4-difluoro-benzyloxy)-phenyl]-5-methyl-pyrrol-1-yl}-5-dimethylamino-benzoic acid, using 2.6 equivalents of ethyl iodide (52 mg, 21%).

¹H NMR (400 MHz, CDCl₃) 1.31 (3H, t, J=6 Hz), 2.16 (3H, s), 4.27 (2H, q, J=7 Hz), 4.81 (2H, s), 6.09 (1H, d, J=3 Hz), 6.28 (1H, d, J=3 Hz), 6.43 (1H, t, J=2 Hz), 6.63 (1H, d, J=9 Hz), 6.75-6.84 (2H, m), 6.98-7.10 (2H, m), 7.13 (1H, t, J=1.5 Hz), 7.19 (1H, d, J=3 Hz), 7.23 (1H, t, J=1.5 Hz).

LC/MS t=4.07 min [MH⁺] 497.

### Example 310

### 3-{2-[5-Bromo-2-(2,4-difluoro-benzyloxy)phenyl]-5-methyl-pyrrol-1-yl}-5-(acetyl-methyl amino)-benzoic acid

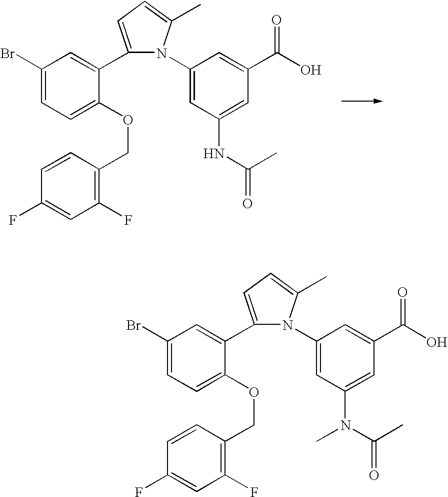

3-{2-[5-Bromo-2-(2,4-difluoro-benzyloxy)-phenyl]-5-methyl-pyrrol-1-yl}-5-acetylamino)-benzoic acid (200 mg, 0.36 mmol)was dissolved in DMF (2.5 ml). Sodium hydride (60% suspension) (40 mg, 0.72 mmol) was added, followed by iodomethane (0.06 mL, 0.8 mmol). The reaction mixture was stirred at room temperature under nitrogen for 4 hours, then water (2 mL) was added and stirring continued overnight. The mixture was further diluted with water (1 0 mL) and extracted with EtOAc, dried (MgSO₄), filtered and evaporated. The residue was chromatographed on a water's sep-pack (10 g) with Et₂O/iso-hexane to give the title compound (48 mg, 23%).

LC/MS t=3.71 min [MH⁺] 571

### Example 311

### 3-{2-[5-Bromo-2-(2,4-difluoro-benzyloxy)-phenyl]-5-methyl-pyrrol-1-yl}-5-pyrrolidin-1-yl-benzoic acid

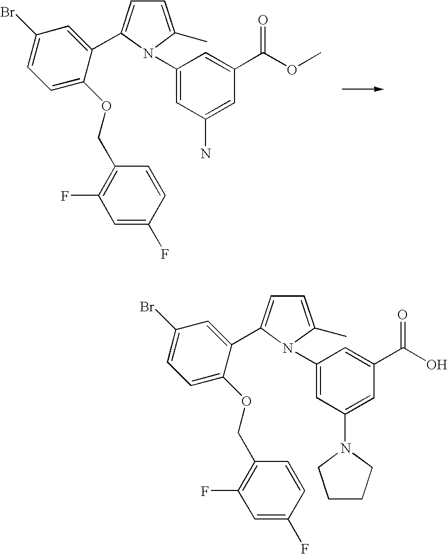

3-{2-[5-Bromo-2-(2,4-difluoro-benzyloxy)-phenyl]-5-methyl-pyrrol-1-yl}5-amino-benzoic acid methyl ester, (187 mg, 0.35 mmol) was dissolved in 2-butanone (2.5 mL). Potassium cabonate (0.700g)and 1,4-dibromobutane (0.2 mL, 1.5 mmol) were added. The mixture was heated at reflux under nitrogen for 72 hrs, cooled, filtered through celite and evaporated. The residue was purified on a Waters sep-pack (10g) with Et₂O/iso-hexane to give the intermediate ester (140 mg, 68%). The free acid was obtained by heating the ester (128 mg, 0.2 mmol) in MeOH (3 mL) and 2M NaOH (2 mL) at 60° C. for 1 hour. Upon cooling, the MeOH was evaporated and the residue was then neutralised with 2M HCl (2 mL) and extracted with EtOAc (10 mL). The organic phase was dried (MgSO₄), filtered and evaporated to give the title compound (1 15 mg, 62%).

¹H NMR (400 MHz, CDCl₃) 1.91-1.98 (4H, m), 2.19 (3H, s), 3.05-3.13 (4H, m), 4.80 (2H, s), 6.11 (1H, d, J=3 Hz), 6.29 (1H, d, J=3 Hz), 6.31 (1H, t, J=2 Hz), 6.56 (1H, d, J=9 Hz), 6.73 -6.81 (2H, m), 6.96-7.03 (1H, m), 7.08 (1H, t, J=1.5 Hz), 7.12 (1H, t, J=1.5 Hz), 7.18-7.22 (1H, dd, J=2 Hz), 7.38 (1H, d, J=2 Hz)

LC/MS t=4.25 mins [MH⁺] 569

### Example 312

### 3-{2-[5-Bromo-2-(2,4-difluoro-benzyloxy)-phenyl]-5-methyl-pyrrol-1-yl}-5-morpholin-1-yl-benzoic acid

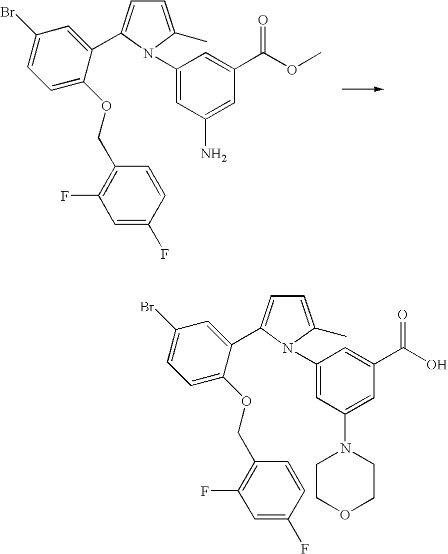

This compound was synthesised in the same way as 3-{2-[5-bromo-2-(2,4-difluoro-benzyloxy)-phenyl]-5-methyl-pyrrol-1-yl}-5-pyrrolidin-1-yl-benzoic acid using the appropriate chloride to give the title compound (1 10 mg, 38%).

¹H NMR (400 MHz, CDCl₃) 2.12 (3H, s), 2.93 (4H, t, J=4 Hz), 3.76 (4H, t, J=4 Hz), 4.75 (2H, s), 6.11 (1H, d, J=3 Hz), 6.29 (1H, d, J=3 Hz), 6.57 (1H, d, J=9 Hz), 6.63 (1H, t, J=2 Hz), 6.73-6.83 (2H, m), 6.95-7.03 (1H, m), 7.23 (1H, dd, J=2 Hz, J=6 Hz), 7.28 (1H, t, J=1.5 Hz), 7.38 (1H, d, J=3 Hz), 7.46 (1H, t, J=1.5 Hz).

LC/MS t=2.96 min [MH⁺] 585

### Example 313

### -3-{2-[5-Bromo-2-(2,4-difluoro-benzyloxy)-phenyl]-5-methyl-pyrrol-1-yl}-6-hydroxy-benzoic acid

### a) 3-amino-6-hydroxy-benzoic-acid-methyl ester

Methyl-2-hydroxy-5-nitrobenzoate (1.000, 5.0 mmol) was stirred under a hydrogen atmosphere at room temperature in methanol (20 ml) with palladium on charcoal for 3 hours. The reaction mixture was filtered through celite and evaporated to give the title compound, which was used without further purification.

LC/MS t=1.54 min [MH⁺] 168

### b) 3-{2-[5-Bromo-2-(2,4-difluoro-benzyloxy)-phenyl]-5-methyl-pyrrol-1-yl}-6-hydroxy-benzoic acid methyl ester

1-[5-Bromo-2-(2,4-difluorobenzyloxy)-phenyl]-pentane-1,4-dione (600 mg, 1.5 mmol), ethyl-3-amino-6-hydroxy-benzoate (800 mg, ˜33% pure, 1.5 mmol), pTSA (cat), and powdered 4A molecular sieves (1.0g) were heated at reflux in toluene (1 0 ml) for 18 hours, cooled, filtered through celite and evaporated. The residue was purified by chromatography on silica gel with Et₂O/iso-hexane, as eluant, to give the title compound (657 mg, 82%).

¹H NMR (400 MHz, CDCl₃) 2.11 (3H, s), 3.84 (3H, s), 4.79 (2H, s), 6.09 (1H, d, J=3 Hz), 6.26 (1H, d, J=3 Hz), 6.58 (1H, d, J=9 Hz), 6.76-6.85 (3H, m), 6.97-7.07 (2H, m), 7.21-7.60 (1H, dd, J=3 Hz, J=7 Hz), 7.37 (1H, d, J=2.5 Hz), 7.48 (1H, d, J=2.5 Hz) 10.40 (1H, br s).

LC/MS t=4.27 min [MH⁺] 530.

### c) 3-{2-[5-Bromo-2-(2,4-difluoro-benzyloxy)-phenyl]-5-methyl-pyrrol-1-yl}-6-hydroxy-benzoic acid

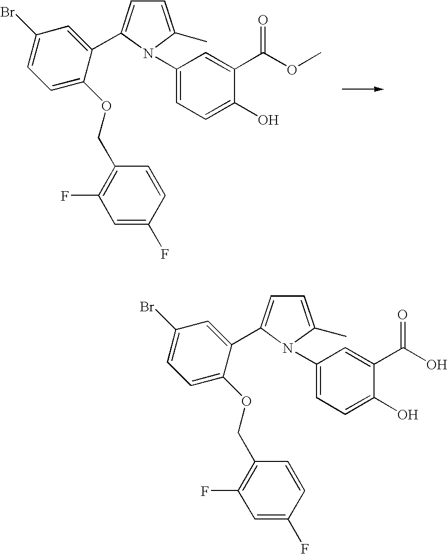

3-{2-[5-Bromo-2-(2,4-difluoro-benzyloxy)-phenyl]-5-methyl-pyrrol-1-yl}-6-hydroxy-benzoic acid methyl ester (180 mg, 0.3 mmol) was heated at 60° C. in MeOH (3 mL) and 2M NaOH (2 mL) for 1 hour. Upon cooling, the solvent volume was reduced to approximately 1.5 mL then diluted with water to approximately 1 0 mL and neutralised with 2M HCl (2 mL). The solution was extracted with EtOAc (10 mL), dried (MgSO₄), filtered and evaporated to give the title compound (45 mg, 26%).

¹H NMR (400 MHz, CDCl₃) 2.12 (3H, s), 4.81 (2H, s), 6.10 (1H, d, J=3 Hz), 6.26 (1H, d, J=3 Hz), 6.59 (1H, d, J=9 Hz), 6.75-6.88 (3H, m), 6.97-7.05 (1H ,m), 7.07-7.12 (1H, dd, J=2.5 Hz, J=6 Hz), 7.21-7.26 (1H, dd, J=2.5, J=6 Hz), 7.36 (1H, d, J=2.5 Hz), 7.54 (1H, d, J=2.5 Hz).

LC/MS t=4.67 min [MH⁺] 514

### Example 314

### 3-{2-[5-Bromo-2-(2,4-difluoro-benzyloxy)-phenyl]-5-methyl-pyrrol-1-yl}-6-methoxy-benzoic acid

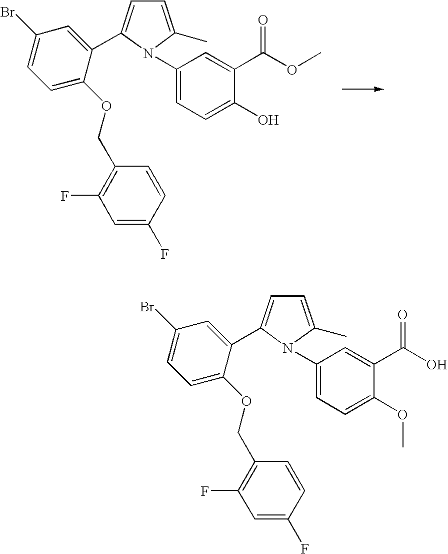

3-{2-[5-Bromo-2-(2,4-difluoro-benzyloxy)-phenyl]-5-methyl-pyrrol-1-yl}-6-hydroxy-benzoic acid methyl ester (260 mg, 0.4 mmol) was dissolved in DMF (3 mL) and treated with sodium hydride (60% suspension) (100 mg, 2.4 mmol) followed by methyl iodide (0.1 mL, 1.6 mmol). The reaction was stirred under nitrogen at room temperature for 2 hours then water (3 mL) was added and stirring continued at room temp overnight. The mixture was further diluted with water (20 mL) extracted with DCM (20 mL). The organic phase was dried (MgSO₄), filtered and evaporated. The residue was purified on a Water's sep-pack cartridge (10g) with Et₂O/iso-hexane to give the title compound (1 50 mg, 56%).

¹H NMR (400 MHz, CDCl₃) 2.12 (3H, s), 4.06 (3H, s), 4.81 (2H, s), 6.09 (1H, d, J=3 Hz), 6.26 (1H, d, J=3 Hz), 6.59 (1H, d, J=9 Hz), 6.76-6.84 (2H, m), 6.90 (1H, d, J=9 Hz) 7.03-7.11 (1H, m), 7.11-7.16 (1H, dd, J=2.5 Hz, J=6 Hz), 7.19-7.24 (1H, dd, J=2.5, J=6 Hz), 7.31 (1H, d, J=2.5 Hz), 7.93 (1H, d, J=2.5 Hz).

LC/MS t=3.93 min [MH⁺] 530.

### Example 315

### 3-{2-[5-Bromo-2-(2,4-difluoro-benzyloxy)-phenyl]-5-methyl-pyrrol-1-yl}-6-acetyl-amino-benzoic acid

### a) 2-(Acetyl-amino)-5-amino-benzoic acid methyl ester

2-(acetyl-amino)-5-nitro-benzoic acid methyl ester (1.000g, 4.2 mmol) in was stirred in MeOH (20 mL) under a hydrogen atmosphere with palladium on charcoal (5% wet) at room temperature and pressure for 3 hours. The reaction mixture was then filtered through celite and evaporated to give the title compound (0.85g, 98%).

¹H NMR (400 MHz, CDCl₃) 2.19 (3H, s), 3.90 (3H, s), 6.90 (1H, dd, J=3 Hz, J=6 Hz), 7.33 (1H, d, J=3 Hz), 8.47 (1H, d, J=9 Hz), 10.70 (1H,s).

### b)₃-{2-[5-Bromo-2-(2,4-difluoro-benzyloxy)-phenyl]-5-methyl-pyrrol-1-yl}-6-acetyl-amino-benzoic acid

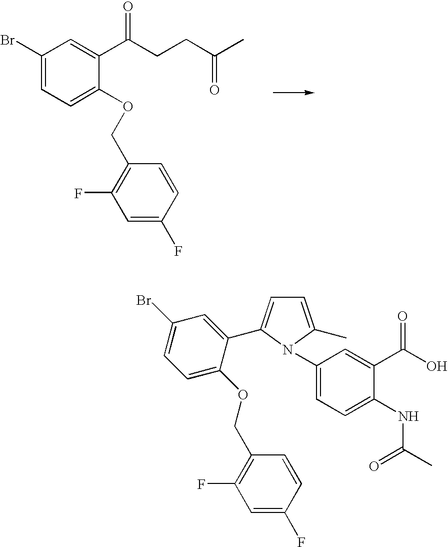

This compound was synthesised and purified in the same way as 3-{2-[5-bromo-2-(2,4-difluorobenzyloxy)-phenyl]-5-methyl-pyrrol-1-yl}-6-hydroxy-benzoic acid using the appropriate amine to give the title compound (220 mg, 83%).

¹H NMR (400 MHz, CDCl₃) 2.13 (3H, s), 2.24 (3H, s), 4.76 (2H, s), 6.11 (1H, d, J=3 Hz) 6.26 (1H, d, J=3 Hz), 6.58 (1H, J=9 Hz), 6.74-6.83 (2H, m), 6.96-7.04 (1H, m), 7.14-7.18 (1H, dd, J=2.5, J=6.5 Hz), 7.21-7.26 (1H, dd, J=2.5, J=6.5 Hz), 7.39 (1H, d, J=2.5 Hz), 7.73 (1H, d, J=2.5 Hz), 8.61 (1H, d, J=9 Hz) 11.00 (1H, s).

LC/MS t=4.13 mins [MH⁺] 557.

### Example 316

### 3-{2-[5-Bromo-2-(2,4-difluoro-benzyloxy)-phenyl]-5-methyl-pyrrol-1-yl}-5-(1,1-dioxo-1I⁶-isothiazolidin-2-yl)-benzoic acid

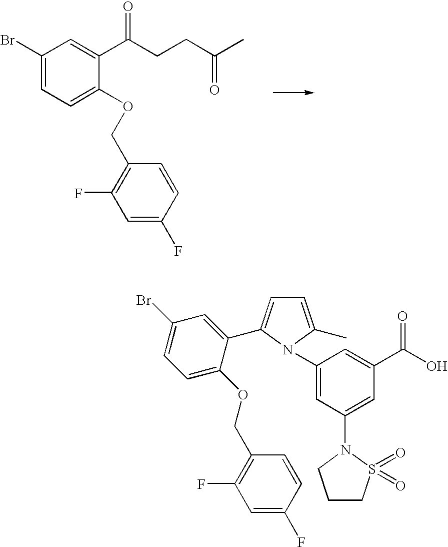

1-[5-Bromo-2-(2,4-difluorobenzyloxy)-phenyl]pentane-1,4-dione (200 mg, 0.5 mmol), 3-amino-5-(1,1-dioxo-1I⁶-isothiazolidin-2-yl)-benzoic acid-methyl ester (140 mg, 0.5 mmol), pTSA (cat) and powdered 4A molecular sieves (1.0g) were heated NMP (2.5 mL) at 180° C. for 18 hours, cooled to room temperature, diluted with EtOAc (15 mL), filtered through celite and washed with EtOAc (5 mL). The filtrate was then washed with water (20 mL), dried (MgSO₄), filtered and evaporated. The residue was purified on a Waters sep-pack (10 g) with Et₂₀₁ iso-hexane to give the intermediate ester. The title compound was then obtained by heating the ester in MeOH and 2N NaOH at 60° C. for 1 hour. Upon cooling, work up gave the title compound (150 mg, overall 50%).

¹H NMR (400 MHz, CDCl₃) 2.20(3H,s),2.46-2.54(2H,m),3.35(2H,t,J=7.5 Hz), 3.57(2H,t,J=6.5 Hz),6.13(1H,d,J=3.0 Hz),6.27(1H,d,J=3.0 Hz),6.58(1H,d,J=9.0 Hz), 6.72-6.83(2H,m),6.94-7.02(1H,m),7.19(1H,t,J=2.0 Hz),7.4(1H,dd,J=3,7 Hz), 7.39(1H,d,J=3.0 Hz),7.5(1H,t,J=1.5 Hz),7.73(1H,t,J=1,5 Hz)

LC/MS t−3.81 mins [MH+] 618.9

### Example 317

### 3-{2-[5-Bromo-2-(2,4-difluoro-benzyloxy)phenyl]-5-methyl-pyrrol-1-yl}-5-(2-oxo-piperidin-1-yl)-benzoic acid

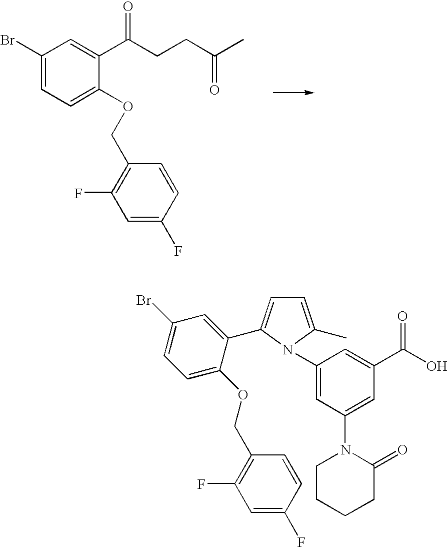

This compound was synthesised and purified in the same way as 3-{2-[5-bromo-2-(2,4-difluoro-benzyloxy)-phenyl]-5-methyl-pyrrol-1-yl}-5-(1,1-dioxo-1I⁶-isothiazolidin-2-yl)-benzoic acid using the appropriate amine to give the title compound (100 mg, overall 33%).

¹H NMR (400 MHz, CDCl₃) 1.83-1.94 (4H, m), 2.20 (3H, s), 2.50-2.57 (2H, m), 3.26-3.33 (2H, m), 4.74 (2H, s), 6.11 (1H, d, J=4 Hz), 6,28 (1H, d, J=4 Hz), 6.57 (1H, d, J=9 Hz), 6.72-6.84 (2H, m), 6.97-7.07 (2H, m), 7.22 (1H, d, J=3.5 Hz), 7.36 (1H, d, J=3 Hz), 7.58 (1H, t, J=1.5 Hz), 7.85 (1H, t, J=1.5 Hz).

LC/MS t=3.88 min [MH⁺] 597.

### Example 318

### 3-{2-[5-Bromo-2-(2,4-difluoro-benzyloxy)-phenyl]-5-methyl-pyrrol-1-yl}-5-methylamino-benzoic acid

### a) 3-Methylamino-5-nitro-benzoic acid methyl ester

3-Amino-5-nitro-benzoic acid (4.000 g, 22 mmol), potassium carbonate (8.000 g) treated and dimethyl sulphate (4.6 ml, 48 mmol) were heated at 80° C. in NMP (25 mL) for 3 hours. The mixture was then cooled, filtered through celite and the residues were washed with DCM (50 ml). The filtrate was evaporated to a thick oil which was dissolved in EtOAc (200 mL) and washed with 0.880 ammonia (100 mL)/water(100 mL). The organic layer was dried, filtered and revaporated to give an oil which was chromatographed on silica gel with Et₂O/iso-hexane to give the title compound (1.600g, 35%).

¹H NMR (400 MHz, CDCl₃) 2.95 (3H, d, J=5 Hz), 3.95 (3H, s), 7.51-7.56 (2H,m), 8.14 (1H, t, J=2 Hz).

### b) 3-Amino-5-methylamino-benzoic acid-methyl ester

3-Methylamino-5-nitro-benzoic-acid-methyl ester (2.200g, 1 0 mmol) stirred under a hydrogen atmosphere in MeOH (100 mL) with 10% palladium on charcoal (0.8g) for 3 hours at 50° C. and 50 psi. The reaction mixture was filtered through celite and evaporated. The residue was purified by chromatography on silica gel with Et₂O containing MeOH (0-10%), as eluant, to give the title compound (0.790g, 42%).

¹H NMR (400 MHz, CDCl₃) 2.84 (3H, s), 3.87 (3H, s), 6.11 (1H, t, J=4 Hz), 6.73 (2H, t, J=4 Hz).

LC/MS t=1.20 mins [MH+] 181.

### c) 3-{2-[5-Bromo-2-(2,4-difluoro-benzyloxy)-phenyl]-5-methyl-pyrrol-1-yl}-5-methylamino-benzoic acid

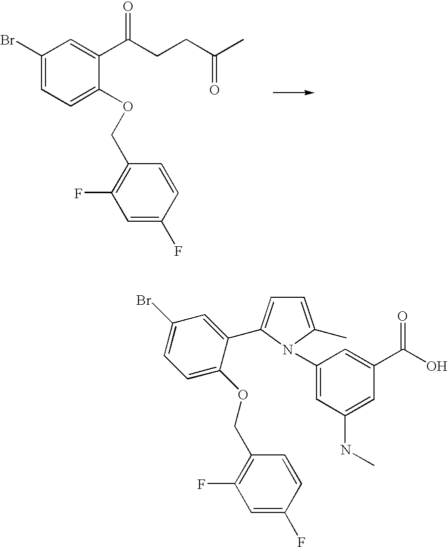

This compound was synthesised and purified in the same way as 3-{2-[5-bromo-2-(2,4-difluoro-benzyloxy)-phenyl]-5-methyl-pyrrol-1-yl}-6-hydroxy-benzoic acid using the appropriate amine to give the title compound (100 mg, 38%).

¹H NMR (400 MHz, CDCl₃) 2.18 (3H, s), 2.69 (3H, s), 4.79 (2H, s), 6.11 (1H, d, J=3 Hz), 6.29 (1H, d, J=3 Hz), 6.36 (1H, t, J=2 Hz), 6.58 (1H, d, J=9 Hz), 6.72-6.84 (2H, m), 6.98-7.07 (1H, m), 7.13 (1H, t, J=1.5 Hz), 7.18 (1H, t, J=1.5 Hz), 7.21 (1H, dd, J=2 Hz, J=6 Hz), 7.37 (1H, d, J=2 Hz).

LC/MS t=3.92 min [MH⁺] 529.

### Example 319

### 3-{2-[5-Chloro-2-(2,4-difluoro-benzyloxy)-phenyl]-5-methyl-pyrrol-1-yl}-5-methylamino-benzoic-acid

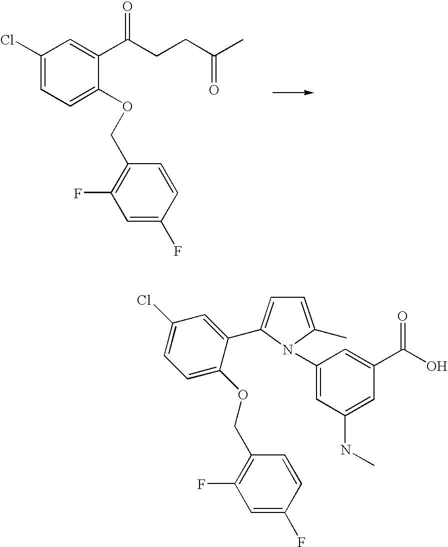

This compound was synthesised and purified in the same way as 3-{2-[5-bromo-2-(2,4-difluoro-benzyloxy)-phenyl]-5-methyl-pyrrol-1-yl}-5-methylamino-benzoic acid using the appropriate amine to give the title compound (62 mg, overall 25%).

¹H NMR (400 MHz, CDCl₃) 2.19 (3H, s), 2.69 (3H, s), 4.79 (2H, s), 6.11 (1H, d, J=3 Hz), 6.29 (1H, d, J=3 Hz), 6.36 (1H, t, J=2 Hz), 6.63 (1H, d, J=8 Hz), 6.72-6.83 (2H, m), 6.99-7.11 (2H, m) 7.18 (1H, t, J=2 Hz), 7.17 (1H, t, J=2 Hz), 7.22 (1H, d, J=2 Hz).

LC/MS t=3.70 min [MH⁺] 483

### Example 320

### 3-{2-[5-Chloro-2-(4-fluoro-benzyloxy)-phenyl]-5-methyl-pyrrol-1-yl}-5-methylamino-benzoic acid

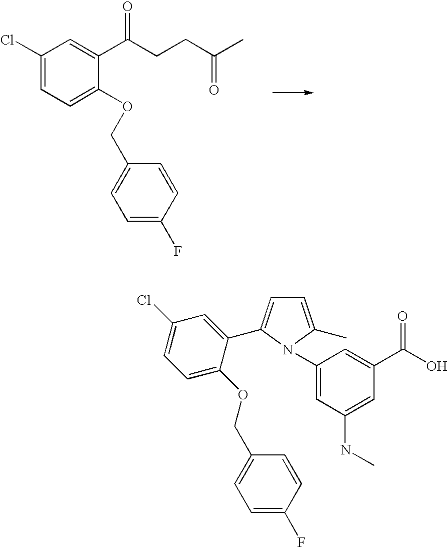

This compound was synthesised and purified in the same way as 3-{2-[5-bromo-2-(2,4-difluoro-benzyloxy)-phenyl]-5-methyl-pyrrol-1-yl}-5-methylamino-benzoic acid using the appropriate amine to give the title compound (54 mg, 23%).

¹H NMR (400 MHz, CDCl₃) 2.18 (3H, s), 2.67 (3H, s), 4.74 (2H, s), 6.12 (1H, d, J=3 Hz), 6.29 (1H, d, J=3 Hz), 6.35 (1H, t, J=2 Hz), 6.58 (1H, d, J=8 Hz), 6.93-7.02 (2H, m), 7.03-7.11 (3H, m) 7.13 (1H, br s), 7.17 (1H, br s), 7.23 (1H, d, J=3 Hz).

LC/MS t=3.86 min [MH⁺] 465.

### Example 321

### 3-{2-[5-Chloro-2-(4-fluoro-benzyloxy)-phenyl]-5-methyl-pyrrol-1-yl}-5-methanesulfonylamido-benzoic acid

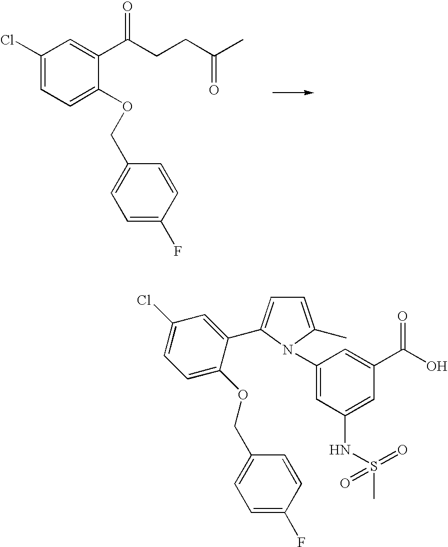

Prepared in the same way as 3-{2-[5-bromo-2-(2,4-difluoro-benzyloxy)-phenyl]-5-methyl-pyrrol-1-yl}-5-propionylamino-benzoic acid using the appropriate amine, to give the title compound (54 mg, 23%).

¹H NMR (400 MHz, CDCl₃) 2.20 (3H, s), 2.70 (3H, s), 4.74 (2H, s), 6.14 (1H, d, J=3 Hz), 6.32 (1H, d, J=3 Hz), 6.62 (1H, d, J=9 Hz), 6.93-7.10 (5H, m), 7.13-7.21 (2H, m), 7.23 (1H, d, J=2 Hz), 7.57 (1H, br s).7.74 (1H, br s).

LC/MS t=3.71 min [MH⁺] 493

### Example 322

### 3-{2-[5-Chloro-2-(2,4-difluoro-benzyloxy)-phenyl]-5-methyl-pyrrol-1-yl}-5-chloro-benzoic acid

### a) 3-Amino-5-chloro benzoic acid

3-Chloro-5-nitro-benzoic acid (1.590g, 7.9 mmol) was stirred under a hydrogen atmosphere in ethanol (40 mL) with raney-nickel, (aqueous suspension, ˜0.4g) at room temperature and pressure for 18 hours. The reaction mixture was then filtered through celite and evaporated to give the title compound (1.300g, 96%).

¹H NMR (400 MHz, CDCl₃) 6.86 (1H, s), 7.18 (1H, s), 7.22 (1H, s).

LC/MS t=2.30 mins [MH⁻] 170.

### b) 3-{2-[5-Chloro-2-(2,4-di-fluoro-benzyloxy)-phenyl]-5-methyl-pyrrol-1yl}-5-chloro-benzoic acid

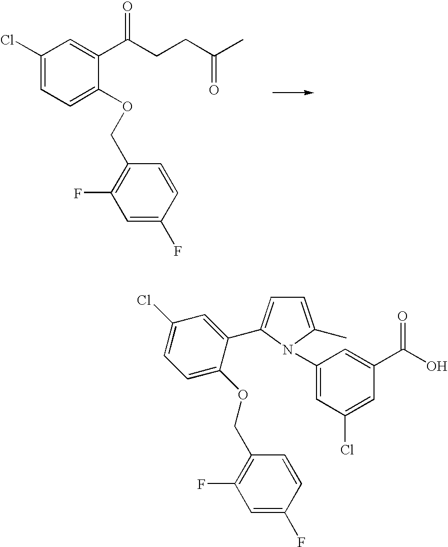

1-[5-Chloro-2-(2,4-difluoro-benzyloxy)-phenyl] pentane-1,4-dione (176 mg, 0.5 mmol) was heated at reflux in toluene (2.0 mL), containing NMP (0.5 mL), with 3-amino-5-chloro-benzoic acid (86 mg, 0.5 mmol), pTSA (cat.) and powdered 4A molecular sieves (0.8 mg) for 18 hours. Upon cooling, the mixture was filtered through celite and washed with EtOAc (10 mL). The filtrate was washed with brine, dried (MgSO₄), filtered and evaporated to an oil which was purified on a Waters sep-pack (10g) with Et₂₀₁ iso-hexane to give the title compound (170 mg, 70%).

¹H NMR (400 MHz, CDCl₃) 2.16 (3H, s), 4.75 (2H, s), 6.13 (1H, d, J=3 Hz), 6.27 (1H, d, J=3 Hz), 6.66 (1H, d, J=9 Hz), 6.73-6.87 (2H, m), 6.98-7.06 (1H, m), 7.10-7.19 (2H, m), 7.25 (1H, s), 6,60 (1H, br s), 7.93 (1H, br s).

LC/MS t=4.40 min [MH⁺] 488.

### Example 323

### 3-{2-[5-Chloro-2-(4-fluoro-benzyloxy)-phenyl]5-methyl-pyrrol-1-yl}-5-chloro-benzoic acid

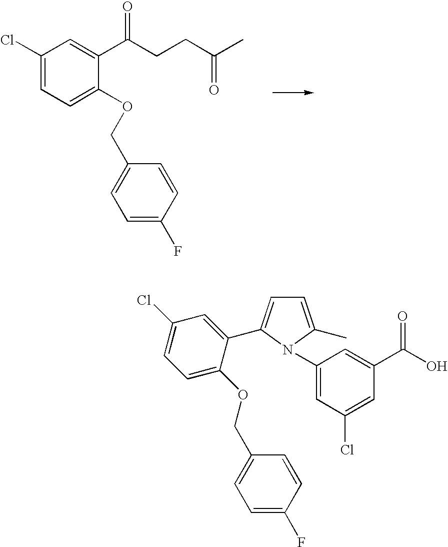

This compound was synthesised and purified in the same way as 3-{2-[5-chloro-2-(2,4-di-fluoro-benzyloxy)-phenyl]-5-methyl-pyrrol-1yl}-5-chloro-benzoic acid using the appropriate amine to give the title compound (150 mg, 64%).

¹H NMR (400 MHz, CDCl₃) 2.16 (3H, s), 4.68 (2H, s), 6.13 (1H, d, J=3 Hz), 6.26 (1H, d, J=3 Hz), 6.62 (1H, d, J=9 Hz), 6.94-7.15 (6H, m), 7.27 (1H, d, J=2 Hz), 7.57 (1H, br s), 7.91 (1H, br s).

LC/MS t=4.00 min [MH⁺] 470.

### Example 324

### 3-{2-[5-Chloro-2-(2,4-difluoro-benzyloxy)-phenyl]-5-methyl-pyrrol-1-yl}-5-bromo-benzoic acid

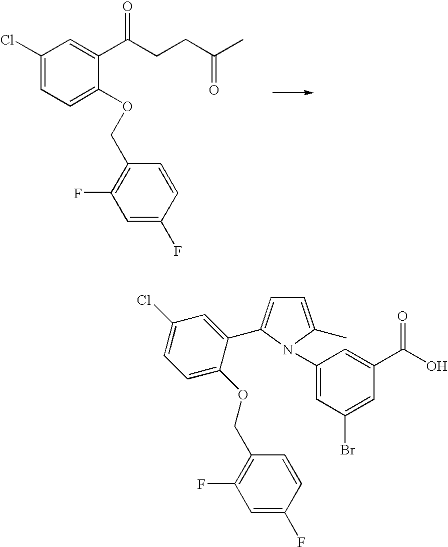

This compound was synthesised and purified in the same way as 3-{2-[5-chloro-2-(2,4-di-fluoro-benzyloxy)-phenyl]-5-methyl-pyrrol-1yl}-5-chloro-benzoic acid using the appropriate amine to give the title compound (75 mg, 29%).

¹H NMR (400 MHz, CDCl₃) 2.16 (3H, s), 4.75 (2H, s), 6.13 (1H, d, J=3 Hz), 6.28 (1H, d, J=3 Hz), 6.66 (1H, d, J=9 Hz), 6.74-6.86 (2H, m), 6.97-7.05 (1H, m), 7.13 (1H, dd, J=2 Hz, J=6 Hz), 7.26 (1H, s), 7.32 (1H, t, J=2 Hz), 7.63 (1H, t, J=1.5 Hz), 8.10 (1H, t, J=1.5 Hz).

LC/MS t=4.10 mins [MH⁺] 534

### Example 325

### 3-{2-[5-Bromo-2-(2,4-difluoro-benzyloxy)-phenyl]-5-methyl-pyrrol-1-yl}-5-chloro-benzoic acid

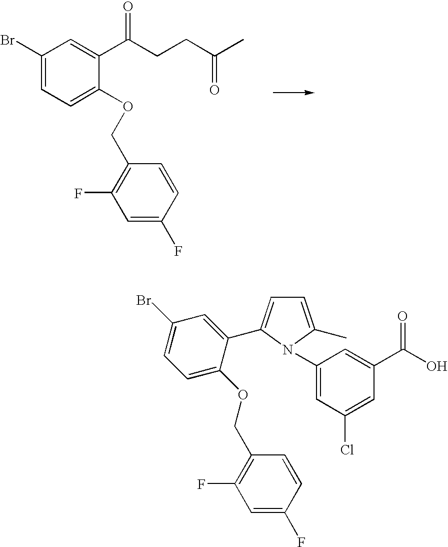

This compound was synthesised and purified in the same way as 3-{2-[5-chloro-2-(2,4-di-fluoro-benzyloxy)-phenyl]-5-methyl-pyrrol-1yl}-5-chloro-benzoic acid using the appropriate amine to give the title compound (70 mg, 26%).

¹H NMR (400 MHz, CDCl₃) 2.15 (3H, s), 4.75 (2H, s), 6.13 (1H, d, J=3 Hz), 6.27 (1H, d, J=3 Hz), 6.61 (1H, d, J=9 Hz), 6.74-6.85 (2H, m), 6.97-7.06 (1H, m) 7.16 (1H, t, J=2 Hz), 7.24-7.30 (2H, m), 7.39 (1H, d, J=2 Hz), 7.59 (1H, t, J=1.5 Hz).

LC/MS t=4.10 mins[MH⁺] 534.

### Example 326

### 3-{2-[5-Chloro-2-(4-fluoro-benzyloxy)-phenyl]-5-methyl-pyrrol-1-yl}-5-bromo-benzoic acid

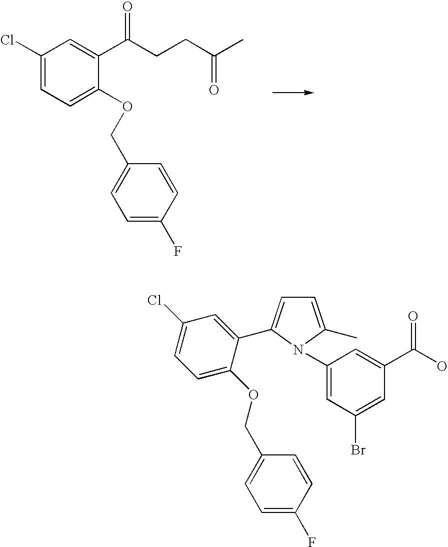

This compound was synthesised and purified in the same way as 3-{2-[5-chloro-2-(2,4-di-fluoro-benzyloxy)-phenyl]-5-methyl-pyrrol-1yl}-5-chloro-benzoic acid using the appropriate amine to give the title compound (80 mg, 31%).

¹H NMR (400 MHz, CDCl₃) 2.15(3H, s), 4.68 (2H, s), 6.13 (1H, d, J=3 Hz), 6.28 (1H, d, =3 Hz), 6.61 (1H, d, J=8 Hz), 6.94-7.14 (5H,m), 7.24-7.32 (2H,m), 7.61 (1H, br s), 8.60 (1H, br s).

LC/MS t=4.32 min [MH⁺] 516.

### Example 327

### 3-{2-[5-Chloro-2-(2,4-difluoro-benzyloxy)-phenyl]-5-methyl-pyrrol-1yl}-5-morpholino-4-yl-benzoic acid

### a) 3-Morpholino-5-nitro-benzoic acid

3-Bromo-5-nitro-benzoic acid (4.000g, 16.3 mmol), cesium carbonate, (8g, 2.46 mmol), tris(dibenzylideneacetone) dipalladium (0) (60 mg, 0.065 mmol), xantphos (116 mg, 0.2 mmol) and morpholine (2.1 ml, 24 mmol) were heated at reflux in dioxan (40 mL) under nitrogen for 92 hours. The reaction mixture was then evaporated to an oil. Upon addition of DCM (˜400 ml) a precipitate formed. The precipitate was removed by filtration and the dissolved in MeOH/DCM (20/40 mL), adsorbed onto silica gel (25g) and chromatographed on silica gel with DCM-10% AcOH in MeOH to give the title compound (2.500g, 61%).

¹H NMR (400 MHz, CDCl₃) 3.30 (4H, t, J=5 Hz), 3.86 (4H, t, J=5 Hz), 7.91 (2H, d, J=8 Hz) 8.18 (1H, s).

LC/MS t=2.60 min [MH⁺] 253.

### b) 3-Amino-5-morpholino-benzoic acid

3-Morpholino-5-nitro-benzoic acid (2.500g, 9.9 mmol) was heated at 50° C. and 50 lb./sq.inch under a hydrogen atmosphere in MeOH (100 mL) with raney-nickel (˜0.5g) for 25 hours. The reaction mixture was then filtered through celite and evaporated. Trituation with ether/iso-hexane (10/40 ml) gave the title compound (1.500g, 77%).

¹H NMR (400 MHz, CDCl₃) 3.12 (4H, t, J=8 Hz), 3.82 (4H, t, J=8 Hz), 6.55 (1H, s), 6.91 (1H, s), 6.97 (1H, s).

LC/MS t=1.40 min [MH⁺] 223.

### c) 3-{2-[5-Chloro-2-(2,4-difluoro-benzyloxy)-phenyl]-5-methyl-pyrrol-1-yl}-5-morpholino-4-yl-benzoic acid

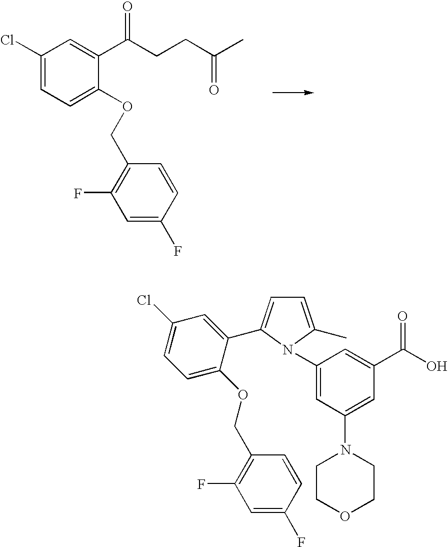

This compound was synthesised and purified in the same way as 3-{2-[5-chloro-2-(2,4-di-fluoro-benzyloxy)-phenyl]-5-methyl-pyrrol-1yl}-5-chloro-benzoic acid using the appropriate amine to give the title compound (120 mg, 45%).

¹H NMR (400 MHz, CDCl₃) 2.20 (3H, s), 2.94 (4H, t, J=5 Hz), 3.76 (4H, t, J=5 Hz), 4,75 (2H, s), 6.13 (1H, d, J=3 Hz), 6.23 (1H, d, J=3 Hz), 6.58-6.67 (2H, m), 6.73-6.84 (2H, m), 6.96-7.05 (1H, m), 7.09 (1H, dd, J=2 Hz, J=6 Hz), 7.23 (1H, d, J=2 Hz), 7.29 (1H, br s), 7.47 (1H, br s).

LC/MS t=3.70 min [MH+] 539.

### Example 328

### 3-{2-[5-Chloro-2-(4-fluoro-benzyloxy)-phenyl]-5-methyl-pyrrol-1-yl}-5-morpholin-4-yl-benzoic-acid

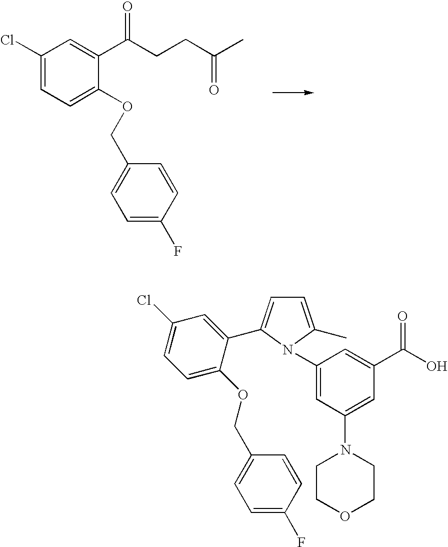

This compound was synthesised and purified in the same way as 3-{2-[5-chloro-2-(2,4-di-fluoro-benzyloxy)-phenyl]-5-methyl-pyrrol-1yl}-5-chloro-benzoic acid using the appropriate amine to give the title compound (100 mg, 38%).

¹H NMR (400 MHz, CDCl₃) 2.20 (3H, s), 2.91 (4H, t, J=5 Hz), 3.75 (4H, t, J=5 Hz) 6.13 (1H, d, J=3 Hz), 6.30 (1H, d, J=3 Hz), 6.58 (1H, d, J=8 Hz), 6.64 (1H, br s), 6.93-7.10 (5H, m), 7.22-7.32 (2H ,m), 7.46 (1H, br s).

LC/MS t=3.70 min [MH⁺] 521.

### Example 329

### 3-{2-[5-Chloro-2-(benzyloxy)-phenyl]-5-methyl-pyrrol-1-yl}-benzoic acid

### a) 1-[5-Chloro-2-(benzyloxy)-phenyl]-hexane-1,4-dione

A mixture of 5-chloro-2-benzyloxy-benzaldehyde (1.003g, 4.07 mmol), ethyl vinyl ketone (0.61 ml), 3-ethyl-5-(2-hydroxyethyl)-4-methylthiazolium bromide (161 mg) and triethylamine (0.85 ml) was heated in ethanol (1.4 ml) at reflux for 5 hours. Upon cooling, the mixture was diluted with EtOAc and washed sequentially with saturated NH₄Cl and saturated NaHCO₃, dried (Na₂SO₄) filtered and concentrated. The residue was purified by chromatography with iso-hexane containing a gradient of EtOAc (5-10%) to give the title compound (0.707g, 52%).

¹H NMR (400 MHz, CDCl₃) 1.06 (3H, t, J=7 Hz), 2.47 (2H, q, J=7 Hz), 2.75 (2H, t, J=7 Hz), 3.24 (2H, t, J=7 Hz), 5.15 (2H, s), 6.95 (1H, d, J=9 Hz), 7.20-7.50 (6H, m's excess), 7.67-7.75 (1H, m).

### b) 3-{2-[5-Chloro-2-(benzyloxy)-phenyl]-5-ethyl-pyrrol-1-yl}-benzoic acid ethyl ester

1-[5-Chloro-2-(benzyloxy)-phenyl]-hexane-1,4-dione (306 mg, 0.92 mmol), ethyl-3-aminobenzoate (0.17 ml, 1.10 mmol) and pTSA (cat) were heated in toluene (9 ml) at reflux for 24 hours. Upon cooling, the mixture was diluted with EtOAc and washed sequentially with 2M HCl and saturated NaHCO₃, dried (Na₂SO₄), filtered and concentrated. The residue was purified by chromatography with hexane containing a gradient of EtOAc (2-3%), as eluant, to give the title compound (233 mg, 55%).

¹H NMR (400 MHz, CDCl₃) 1.15 (3H, t, J=8 Hz), 1.30 (3H, t, J=8 Hz), 2.47 (2H, t, J=8 Hz), 4.28 (2H, q, J=7 Hz), 4.74 (2H, s), 6.16 (1H, d, J=3 Hz), 6.34 (1H, d, J=3 Hz), 6.53 (1H, d, J=9 Hz), 6.98-7.10 (3H, m), 7.11-7.17 (1H, m), 7.20-7.33 (5H, m′ excess), 7.76 (1H, s), 7.91 (1H, d, J=8 Hz).

### c) 3-{2-[5-Chloro-2-(benzyloxy)-phenyl]-5-ethyl-pyrrol-1-yl}-benzoic acid

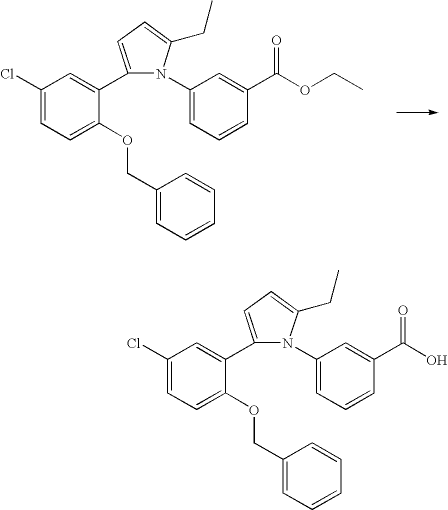

3-{2-[5-Chloro-2-(benzyloxy)-phenyl]-5-ethyl-pyrrol-1-yl}-benzoic acid ethyl ester (233 mg) was heated in EtOH (5 ml) and 2M NaOH (2.5 ml) at reflux for 2 hrs. The mixture was cooled to room temperature and diluted with EtOAc, washed with 2M HCl then dried (Na₂SO₄), filtered and evaporated to give the title compound.

LC/MS t=4.05 min [MH+] 432, 434; [MH−] 430, 432.

### Example 330

### 3-{2-[5-Bromo-2-(benzyloxy)-phenyl]-5-ethyl-pyrrol-1-yl}-benzoic acid

### a) 1-[5-Bromo-2-(benzyloxy)-phenyl]-hexane-1,4-dione

A mixture of 5-bromo-2-benzyloxy-benzaldehyde (1.059g, 3.64 mmol), ethyl vinyl ketone (0.54 ml), 3-ethyl-5-(2-hydroxyethyl)-4-methylthiazolium bromide (173 mg) and triethylamine (0.76 ml) was heated in ethanol (1.2 ml) at reflux for 5 hours. Upon cooling, the mixture was diluted with EtOAc and washed sequentially with saturated NH₄Cl and saturated NaHCO₃, dried (Na₂SO₄) filtered and concentrated. The residue was purified by chromatography with iso-hexane containing a gradient of EtOAc (5-10%) to give the title compound (0.813 g).

¹H NMR (400 MHz, CDCl₃) 1.05 (3H, t, J=7 Hz), 2.47 (2H, q, J=7 Hz), 2.74 (2H, t, J=7 Hz), 3.24 (2H, t, J=7 Hz), 5.14 (2H, s), 6.90 (1H, d, J=9 Hz), 7.20-7.56 (6H, m's excess), 7.82-7.88 (1H, m).

### b) 3-{2-[5-Bromo-2-(benzyloxy)-phenyl]-5-ethyl-pyrrol-1-yl}-benzoic acid ethyl ester

1-[5-Bromo-2-(benzyloxy)-phenyl]-hexane-1,4-dione (390 mg, 1.04 mmol), ethyl-3-aminobenzoate (0.19 ml) and pTSA (cat) were heated in toluene (1 0.4 ml) at reflux for 24 hours. Upon cooling, the mixture was diluted with EtOAc and washed sequentially with 2M HCl and saturated NaHCO₃, dried (Na₂SO₄), filtered and concentrated. The residue was purified by chromatography with hexane containing a gradient of EtOAc (2-3%) as eluant, to give the title compound (294 mg, 57%).

¹H NMR (400 MHz, CDCl₃) 1.15 (3H, t, J=7 Hz), 1.31 (3H, t, J=7 Hz), 2.47 (2H, q, J=7 Hz), 4.28 (2H, q, J=7 Hz), 4.73 (2H, s), 6.16 (1H, d, J=3 Hz), 6.34 (1H, d, J=3 Hz), 6.48 (1H, d, J=9 Hz), 7.00-7.07 (2H, m), 7.10-7.20 (2H, m), 7.22-7.33 (4H, m′ excess), 7.38 (1H, s), 7.76 (1H, s), 7.91 (1H, d, J=8 Hz).

### c) 3-{2-[5-Bromo-2-(benzyloxy)-phenyl]-5-ethyl-pyrrol-1-yl}-benzoic acid

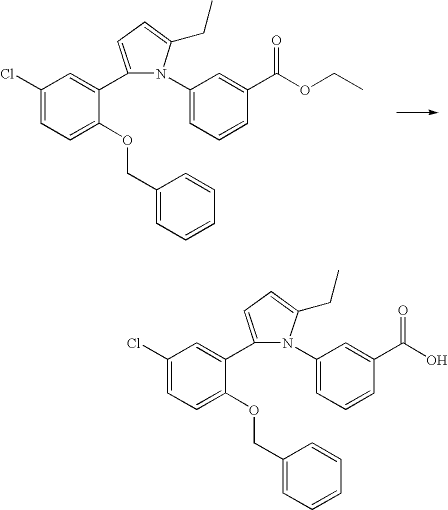

3-{2-[5-Bromo-2-(benzyloxy)-phenyl]-5ethyl-pyrrol-1-yl}-benzoic acid ethyl ester (233 mg) was heated in EtOH (6 ml) and 2M NaOH (3 ml) at reflux for 2 hrs. The mixture was cooled to room temperature and diluted with EtOAc, washed with 2M HCl then dried (Na₂SO₄), filtered and evaporated to give the title compound.

LC/MS t=4.08 min [MH+] 476,478; [MH−] 474, 476.

### Example 331

### 3-{2-[5-Methyl-2-(4-fluoro-benzyloxy)-phenyl]-5-methyl-pyrrol-1-yl}-5-trifluoromethyl-benzoic acid

### a) 5-Methyl-2-(4-fluoro-benzyloxy)-benzaldehyde

5-Methyl-2-hydroxy-benzaldehyde (5.000g, 36.8 mmol), K₂CO₃ (10.212g) and 4-fluorobenzyl bromide (4.81 mL, 38.61 mmol) were heated at reflux in acetone (37 mL) for 1.75 hrs. Upon cooling to room temperature, the residue was diluted with acetone, filtered and evaporated. The residue was dissolved in Et₂O, washed with water, dried (Na₂SO₄), filtered and evaporated to give the title compound.

¹H NMR (400 MHz, CDCl₃) 2.31 (3H, s), 5.12 (2H, s), 6.93 (1H, d, J=6.9 Hz), 7.02-7.15 (2H, m), 7.34 (1H, dd, J=2 Hz, J=9 Hz), 7.37-7.45 (2H, m), 7.65 (1H, d, J=9 Hz) 10.52 (1H, s).

### b) 1-[5-Methyl-2-(4-fluoro-benzyloxy)-phenyl]-pentane-1,4-dione

A mixture of 5-methyl-2-(4-fluoro-benzyloxy)-benzaldehyde (4.960g, 20.34 mmol), methyl vinyl ketone (2.54 ml, 30.60 mmol), 3-ethyl-5-(2-hydroxyethyl)+methylthiazolium bromide (837 mg) and triethylamine (4.3 ml) was heated in ethanol (7.0 ml) at reflux for 5 hours. Upon cooling, the mixture was diluted with EtOAc and washed sequentially with saturated NH₄Cl and saturated NaHCO₃, dried (Na₂SO₄) filtered and concentrated. The residue was purified by chromatography with iso-hexane containing a gradient of EtOAc (2-15%) to give the title compound (2.108g, 35%).

¹H NMR (400 MHz, CDCl₃) 2.19 (3H, s), 2.30 (3H, s), 2.78 (2H, t, J=6 Hz), 3.22 (2H, t, J=6 Hz), 5.10 (2H, s), 6.89 (1H, d, J=9 Hz), 7.05-7.13 (2H, m), 7.23 (1H, dd, J=2 Hz, J=9 Hz), 7.38-7.47 (2H, m), 7.53 (1H, d, J=2 Hz).

### c) 3-{2-[5-Methyl-2-(4-fluoro-benzyloxy)-phenyl]-5-methyl-pyrrol-1-yl}-5-trifluoromethyl-benzoic acid methyl ester

1-[5-Methyl-2-(4-fluoro-benzyloxy)-phenyl]-pentane-1,4-dione (11 0 mg), 3-amino-5-trifluoromethyl-benzoic acid methyl ester (96 mg) and pTSA (cat) were heated in NMP at 150° C. in a microwave for 10 minutes. The mixture was diluted with EtOAc and washed sequentially with 2M HCl and saturated NaHCO₃, dried (Na₂SO₄), filtered and concentrated. The residue was purified on silica gel with iso-hexane containing EtOAc (3-6%) to give the title compound.

LC/MS t=4.28 min, [MH⁺] 498.

### d) 3-{2-[5-Methyl-2-(4-fluoro-benzyloxy)-phenyl]-5-methyl-pyrrol-1-yl}-5-trifluoromethyl-benzoic acid

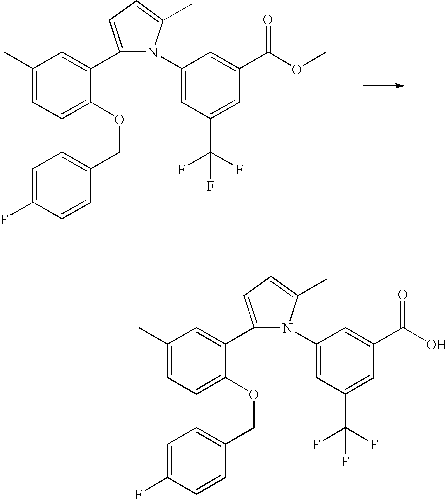

3-{2-[5-Methyl-2-(4-fluoro-benzyloxy)-phenyl]-5-methyl-pyrrol-1-yl}-5-trifluoromethyl-benzoic acid methyl ester (76 mg) was heated at 120° C. in a microwave in a mixture of EtOH (1.6 mL) and 2M NaOH (0.8 mL) for 3 minutes. The mixture was diluted with DCM and 2M HCl and filtered through a hydrophobic frit, fitted with a plug of Na₂SO₄, and evaporated. The residue was purified by MDAP to give the title compound (55 mg).

LC/MS t=4.19 min, [MH⁺] 484; [MH⁻] 482.

### Example 332

### 3-{2-[5-Methyl-2-(4-fluoro-benzyloxy)-phenyl]-5-methyl-pyrrol-1-yl}-6-difluoromethoxy-benzoic acid

### a) 3-{2-[5-Methyl-2-(4-fluoro-benzyloxy)-phenyl]-5-methyl-pyrrol-1-yl}-6-difluoromethoxy-benzoic acid methyl ester

1-[5-Methyl-2-(4-fluoro-benzyloxy)-phenyl]-pentane-1,4-dione (107 mg), 4amino-2-difluoromethoxy-benzoic acid methyl ester (76 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (5-10%) to give the title compound (70 mg, 40%).

LC/MS t=3.84 min, [MH⁺] 496.

### b) 3-{2-[5-Methyl-2-(4-fluoro-benzyloxy)-phenyl]-5-methyl-pyrrol-1-yl}-6-difluoromethoxy-benzoic acid

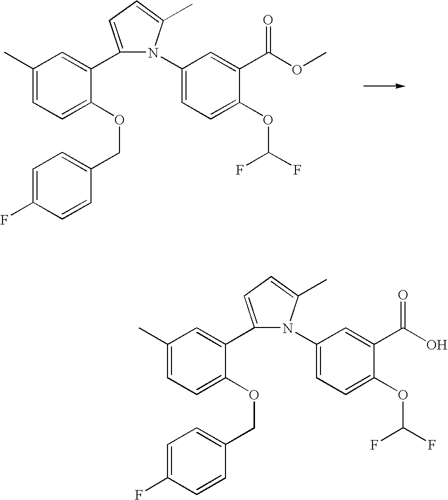

3-{2-[5-Methyl-2-(4-fluoro-benzyloxy)-phenyl]-5-methyl-pyrrol-1-yl}-6-difluoromethoxy-benzoic acid methyl ester (70 mg) was heated at 120° C. in a microwave in a mixture of EtOH (1.4 mL) and 2M NaOH (0.7 mL) for 3 minutes. The mixture was diluted with DCM and 2M HCl and filtered through a hydrophobic frit, fitted with a plug of Na₂SO₄, and evaporated. The residue was purified by MDAP to give the title compound (47 mg).

¹H NMR (400 MHz, d6-DMSO) 2.06 (3H, s), 2.15 (3H, s), 4.75 (2H, s), 6.03 (1H, d, J=3 Hz), 6.12 (1H, d, J=3 Hz), 6.72 (1H, d, J=9 Hz), 6.92-6.98 (2H, m), 6.98-7.38 (7H, m), 7.41 (1H, d, J=2 Hz), 13.11 (1H, br).

LC/MS t=3.95 min, [MH⁺] 482; [MH⁻] 480.

### Example 333

### 3-{2-[5-Methyl-2-(4-fluoro-benzyloxy)-phenyl]-5-methyl-pyrrol-1-yl}-5-(2-oxo-pyrrolidin-1-yl)-benzoic acid

a) 3-{2-[5-Methyl-2-(4-fluoro-benzyloxy)-phenyl]-5-methyl-pyrrol-1-yl}-5-(2-oxo-pyrrolidin-1-yl)-benzoic acid methyl ester

1-[5-Methyl-2-(4-fluoro-benzyloxy)-phenyl]-pentane-1,4-dione (104 mg), 3-amino-5-(2-oxo-pyrrolidin-1yl)-benzoic acid methyl ester (94 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (20-50%) to give the title compound (81 mg, 45%).

LC/MS t=3.68 min, [MH⁺] 513

### b) 3-{2-[5-Methyl-2-(4-fluoro-benzyloxy)-phenyl]-5-methyl-pyrrol-1-yl}-5-(2-oxo-pyrrolidin-1-yl)-benzoic acid

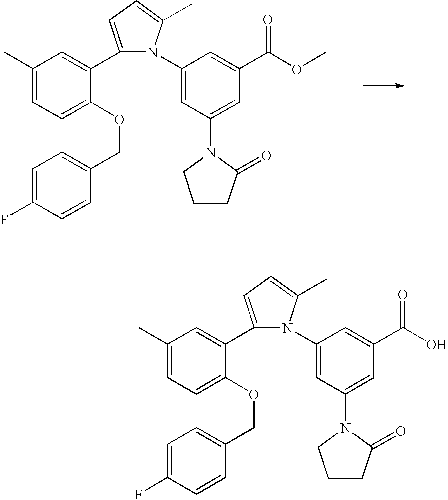

3-{2-[5-Methyl-2-(4-fluoro-benzyloxy)-phenyl]-5-methyl-pyrrol-1-yl}-5-(2-oxo-pyrrolidin-1-yl)-benzoic acid methyl ester (80 mg) was heated at 120° C. in a microwave in a mixture of EtOH (1.6 mL) and 2M NaOH (0.8 mL) for 3 minutes. The mixture was diluted with DCM and 2M HCl and filtered through a hydrophobic frit, fitted with a plug of Na₂SO₄, and evaporated. The residue was purified by MDAP to give the title compound.

LC/MS t=3.71 min, [MH⁺] 499; [MH⁻] 497.

### Example 334

### 3-{2-[5-Methyl-2-(4-fluoro-benzyloxy)-phenyl]-5-methyl-pyrrol-1-yl}-5-(2-oxo-piperidin-1-yl)-benzoic acid

### a) 3-{2-[5-Methyl-2-(4-fluoro-benzyloxy)-phenyl]-5-methyl-pyrrol-1-yl}-5-(2-oxo-piperidin-1-yl)-benzoic acid methyl ester

1-[5-Methyl-2-(4-fluoro-benzyloxy)-phenyl]-pentane-1,4-dione (107 mg), 3-amino-5-(2-oxo-piperidin-1yl)-benzoic acid methyl ester (99 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (20-50%) to give the title compound (88 mg, 46%).

LC/MS t=3.63 min [MH⁺] 527

### b) 3-{2-[5-Methyl-2-(4-fluoro-benzyloxy)-phenyl]-5-methyl-pyrrol-4-yl}-5-(2-oxo-piperidin-1-yl)-benzoic acid

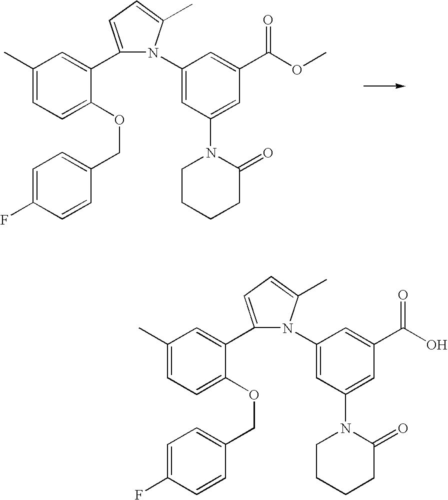

3-{2-[5-Methyl-2-(4-fluoro-benzyloxy)-phenyl]-5-methyl-pyrrol-1-yl}-5-(2-oxo-piperidin-1-yl)-benzoic acid methyl ester (83 mg) was heated at 120° C. in a microwave in a mixture of EtOH (1.6 mL) and 2M NaOH (0.8 mL) for 3 minutes. The mixture was diluted with DCM and 2M HCl and filtered through a hydrophobic frit, fitted with a plug of Na₂SO₄, and evaporated. The residue was purified by MDAP to give the title compound (48 mg).

LC/MS t=3.67 min, [MH⁺] 513; [MH⁻] 511.

### Example 335

### 3-{2-[5-Methyl-2-(4-fluoro-benzyloxy)-phenyl]-5-methyl-pyrrol-1-yl}-5-(1,1-dioxo-1I⁶-isothiazolidin-2-yl)-benzoic acid

### a) 3-{2-[5-Methyl-2-(4-fluoro-benzyloxy)-phenyl]-5-methyl-pyrrol-1-yl}-5-(1,1-dioxo-1I⁶-isothiazolidin-2-yl)-benzoic acid methyl ester

1-[5-Methyl-2-(4-fluoro-benzyloxy)-phenyl]-pentane-1,4dione (110 mg), 3-amino-5-(1,1-dioxo-1I⁶-isothiazolidin-2-yl)-benzoic acid methyl ester (99 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (20-50%) to give the title compound (82 mg, 41%).

LC/MS t=3.63 min [MH⁺] 549

### b) 3-{2-[5-Methyl-2-(4-fluoro-benzyloxy)-phenyl]-5-methyl-pyrrol-1-yl}-5-(1,1-dioxo-1I⁶-isothiazolidin-2-yl)-benzoic acid

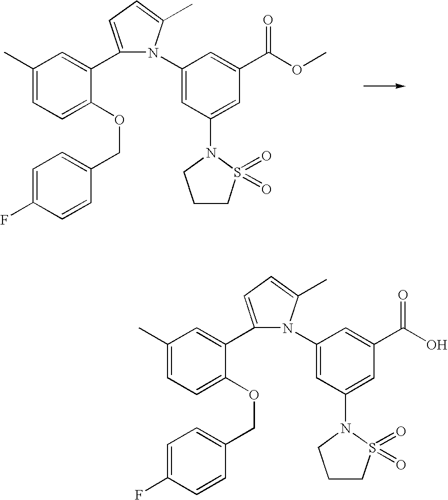

3-{2-[5-Methyl-2-(4-fluoro-benzyloxy)-phenyl]-5-methyl-pyrrol-1-yl}-5-(1,1-dioxo-1I⁶-isothiazolidin-2-yl)-benzoic acid methyl ester (72 mg) was heated at 120° C. in a microwave in a mixture of EtOH (1.6 mL) and 2M NaOH (0.8 mL) for 3 minutes. The mixture was diluted with DCM and 2M HCl and filtered through a hydrophobic frit, fitted with a plug of Na₂SO₄, and evaporated. The residue was purified by MDAP to give the title compound (45 mg).

LC/MS t=3.68 min, [MH⁺] 535; [MH⁻] 533.

### Example 336

### 3-{2-[6-Methyl-2-(4-fluoro-benzyloxy)-phenyl]-5-methyl-pyrrol-1-yl}-6-acetylamino-benzoic acid

### a) 3-{2-[5-Methyl-2-(4-fluoro-benzyloxy)-phenyl]-5-methyl-pyrrol-1-yl}-acetylamino-benzoic acid methyl ester

1-[5-Methyl-2-(4-fluoro-benzyloxy)-phenyl]-pentane-1,4-dione (11 0 mg), 2-acetylamino-5-amino-benzoic acid methyl ester (76 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (12-20%) to give the title compound.

LC/MS t=3.76 min [MH⁺] 487

### b) 3-{2-[5-Methyl-2-(4-fluoro-benzyloxy)-phenyl]-5-methyl-pyrrol-1-yl}-6-acetylamino-benzoic acid

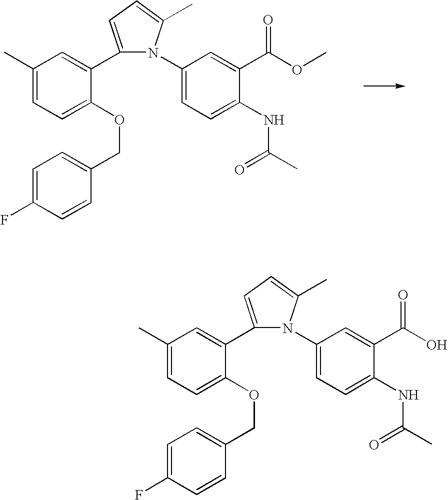

3-{2-[5-Methyl-2-(4-fluorobenzyloxy)-phenyl]-5-methyl-pyrrol-1-yl}-6-acetylamino-benzoic acid methyl ester (82 mg) s heated at 120° C. in a microwave in a mixture of EtOH (1.6 mL) and 2M NaOH (0.8 mL) for 3 minutes. The mixture was diluted with DCM and 2M HCl and filtered through a hydrophobic frit, fitted with a plug of Na₂SO₄, and evaporated. The residue was purified by MDAP to give the title compound (46 mg).

¹H NMR (400 MHz, d6-DMSO) 2.05 (3H, s), 2.12 (3H, s), 2.14 (3H, s), 4.78 (2H, s), 6.01 (1H, d, J=3 Hz), 6.71 (1H, d, J=8 Hz), 6.90-6.98 (2H, m), 7.07-7.25 (6H, m), 7.56 (1H, d, J=2 Hz), 8.33 (1H, d, J=9 Hz), 11.11 (1H, br).

LC/MS t=3.62 min, [MH⁺] 431; [MH⁻] 429.

### Example 337

### 3-{2-[5-Methyl-2-(4-fluoro-benzyloxy)-phenyl]-5-methyl-pyrrol-1-yl}-5-amino-benzoic acid

### a) 3-{2-[5-Methyl-2-(4-fluoro-benzyloxy)-phenyl]-5-methyl-pyrrol-1-yl}-5-amino-benzoic acid methyl ester

1-[5-Methyl-2-(4-fluoro-benzyloxy)-phenyl]-pentane-1,4-dione (608 mg), 3,5-diamino-benzoic acid methyl ester (387 mg) and pTSA (cat) were heated in NMP (4 mL) at 150° C. in a microwave for 10 minutes. The mixture was diluted with EtOAc and washed sequentially with 2M HCl and saturated NaHCO₃, dried (Na₂SO₄), filtered and concentrated. The residue was purified on silica gel with iso-hexane containing EtOAc (12-20%) to give the title compound (478 mg, 50%).

LC/MS t=3.63 min [MH⁺] 445

### b) 3-{2-[5-Methyl-2-(4-fluoro-benzyloxy)-phenyl]-5-methyl-pyrrol-1-yl}-5-amino-benzoic acid

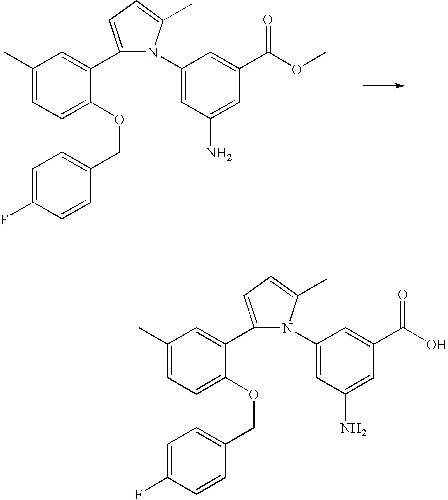

3-{2-[5-Methyl-2-(4-fluoro-benzyloxy)-phenyl]-5-methyl-pyrrol-1-yl}-5-amino-benzoic acid methyl ester (82 mg) was heated at 120° C. in a microwave in a mixture of EtOH (1.6 mL) and 2M NaOH (0.8 mL) for 3 minutes. The mixture was diluted with DCM and 2M HCl and filtered through a hydrophobic frit, fitted with a plug of Na₂SO₄, and evaporated. The residue was purified by MDAP to give the title compound.

¹H NMR (400 MHz, d6-DMSO) 2.59 (3H, s), 2.65 (3H, s), 5.37 (2H, s), 6.53 (1H, d, J=3 Hz), 6.64 (1H, d, J=3 Hz), 7.17 (1H, s), 7.26 (1H, d, J=9 Hz), 7.39-7.50 (3H, m), 7.62-7.82 (5H, m).

LC/MS t=3.62 min, [MH⁺] 431; [MH⁻] 429.

### Example 338

### 3-{2-[5-Methyl-2-(4-fluoro-benzyloxy)-phenyl]-5-methyl-pyrrol-1-yl}-5-amino-6-methyl-benzoic acid

### a) 3-{2-[5-Methyl-2-(4-fluoro-benzyloxy)-phenyl]-5-methyl-pyrrol-1-yl}-5-amino-6-methyl-benzoic acid methyl ester

1-[5-Methyl-2-(4-fluoro-benzyloxy)-phenyl]-pentane-1,4-dione (250 mg), 3,5-diamino-2-methyl-benzoic acid methyl ester (163 mg) and pTSA (cat) were heated in NMP (3.4 mL) at 150° C. in a microwave for 10 minutes. The mixture was diluted with EtOAc and washed sequentially with 2M HCl and saturated NaHCO₃, dried (Na₂SO₄), filtered and concentrated. The residue was purified on silica gel with iso-hexane containing EtOAc (12-20%) to give the title compound (164 mg, 43%).

LC/MS t=3.67 min [MH⁺] 249

### b) 3-{2-[5-Methyl-2-(4-fluoro-benzyloxy)-phenyl]-5-methyl-pyrrol-1-yl}-5-amino-6-methyl-benzoic acid

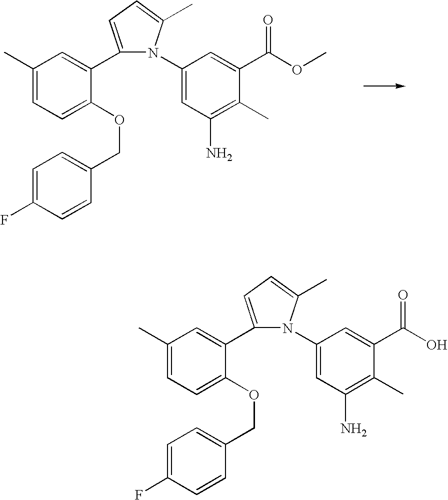

3-{2-[5-Methyl-2-(4-fluoro-benzyloxy)-phenyl]-5-methyl-pyrrol-1-yl}-5-diamino-6-methyl-benzoic acid methyl ester (1 01 mg) was heated at 120° C. in a microwave in a mixture of EtOH (2.0 mL) and 2M NaOH (1.0 mL) for 3 minutes. The mixture was diluted with DCM and 2M HCl and filtered through a hydrophobic frit, fitted with a plug of Na₂SO₄, and evaporated. The residue was purified by MDAP to give the title compound.

LC/MS t=3.64 min, [MH⁺] 445; [MH⁻] 443.

### Example 339

### 3-{2-[5-Methyl-2-(4-fluoro-benzyloxy)-phenyl]-5-methyl-pyrrol-1-yl}-6-methyl-benzoic acid

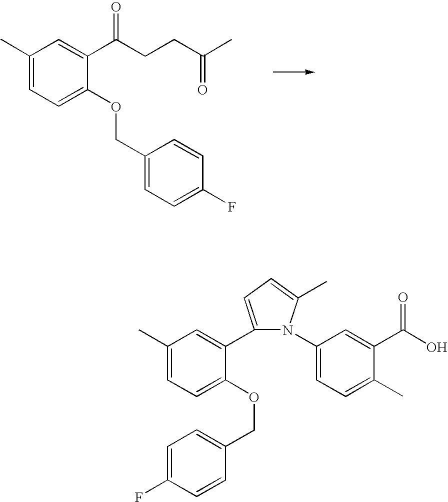

1-[5-Methyl-2-(4-fluoro-benzyloxy)-phenyl]-pentane-1,4-dione, 5-amino-2-methyl-benzoic acid and pTSA (cat) were heated in NMP at 1 50° C. in a microwave for 10 minutes. The mixture was diluted with EtOAc and washed sequentially with 2M HCl and water, dried (Na₂SO₄), filtered and concentrated. The residue was purified by MDAP to give the title compound.

LC/MS t=3.96 min [MH⁺] 430, [MH⁻] 428

### Example 340

### 3-{2-[5-Methyl-2-(4-fluoro-benzyloxy)-phenyl]-5-methyl-pyrrol-1-yl}-6-fluoro-benzoic acid

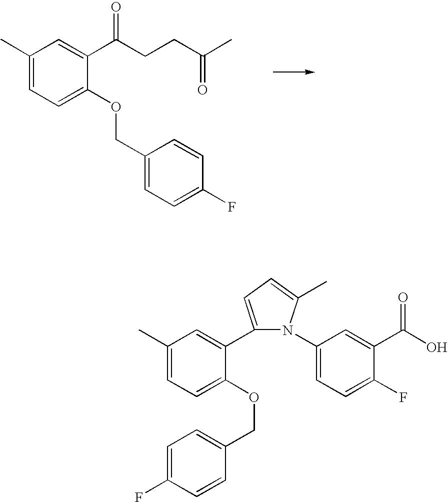

1-[5-Methyl-2-(4-fluoro-benzyloxy)-phenyl]-pentane-1,4-dione, 5-amino-2-fluoro-benzoic acid and pTSA (cat) were heated in NMP at 1 50° C. in a microwave for 10 minutes. The mixture was diluted with EtOAc and washed sequentially with 2M HCl and water, dried (Na₂SO₄), filtered and concentrated. The residue was purified by MDAP to give the title compound.

LC/MS t=3.95 min [MH⁺] 434, [MH⁻] 432

### Example 341

### 3-{2-[5-Methyl-2-(4-fluoro-benzyloxy)-phenyl]-5-methyl-pyrrol-1-yl}-6-chloro-benzoic acid

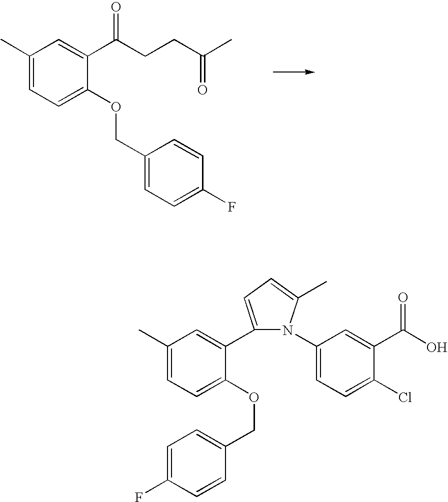

1-[5-Methyl-2-(4-fluoro-benzyloxy)-phenyl]-pentane-1,4-dione, 5-amino-2-chloro-benzoic acid and pTSA (cat) were heated in NMP at 150° C. in a microwave for 10 minutes. The mixture was diluted with EtOAc and washed sequentially with 2M HCl and water, dried (Na₂SO₄), filtered and concentrated. The residue was purified by MDAP to give the title compound.

¹H NMR (400 MHz, CDCl₃) 2.15 (3H, s), 2.25 (3H, s), 4.67 (2H, s), 6.13 (1H, d, J=3 Hz), 6.25 (1H, d, J=4 Hz), 6.56 (1H, d, J=8 Hz), 6.91-7.07 (6H, m), 7.11 (1H, m, J=2 Hz), 7.23 (1H, s), 7.63 (1H, d, J=2 Hz).

LC/MS t=4.14 min [MH⁺] 450, [MH⁻] 448

### Example 342

### 3-{2-[5-Methyl-2-(4-fluoro-benzyloxy)-phenyl]-5-methyl-pyrrol-1-yl}-1-naphthoic acid

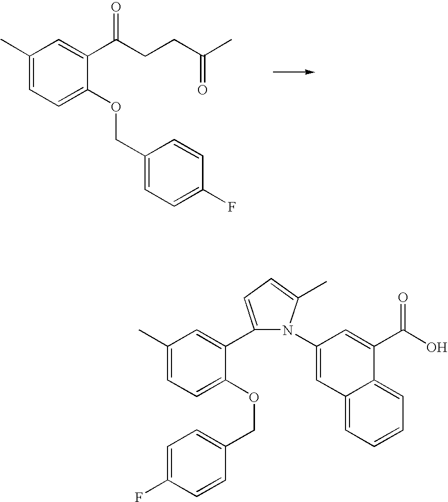

1-[5Methyl-2-(4-fluoro-benzyloxy)-phenyl]-pentane-1,4-dione, 3-amino-naphthalene-1-carboxylic acid and pTSA (cat) were heated in NMP at 150° C. in a microwave for 10 minutes. The mixture was diluted with EtOAc and washed sequentially with 2M HCl and water, dried (Na₂SO₄), filtered and concentrated. The residue was purified by MDAP to give the title compound.

LC/MS t=4.12 min [MH⁺] 466, [MH⁻] 464

### Example 343

### 3-{2-[5-Methyl-2-(4-fluoro-benzyloxy)-phenyl]-5-methyl-pyrrol-1-yl}-5-acetylamino-benzoic acid

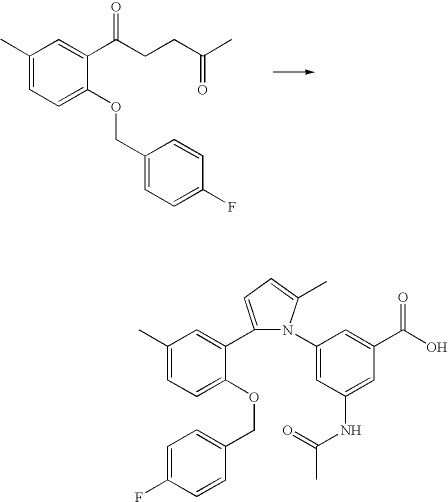

1-[5-Methyl-2-(4-fluoro-benzyloxy)-phenyl]-pentane-1,4-dione, 3-acetylamino-5-amino-benzoic acid and pTSA (cat) were heated in NMP at 150° C. in a microwave for 10 minutes. The mixture was diluted with EtOAc and washed sequentially with 2M HCl and water, dried (Na₂SO₄), filtered and concentrated. The residue was purified by MDAP to give the title compound.

LC/MS t=3.61 min [MH⁺] 473, [MH⁻] 471

### Example 344

### 3-{2-[5-Chloro-2-benzyloxy)-phenyl]-5-methyl-pyrrol-1-yl}-5-trifluoromethyl-benzoic acid

### a) 3-{2-[5-Chloro-2-(benzyloxy)-phenyl]-5-methyl-pyrrol-1-yl}-5-trifluoromethyl-benzoic acid methyl ester

1-[5-Chloro-2-benzyloxy)-phenyl]-pentane-1,4-dione (104 mg), 3-amino-5-trifluoromethyl-benzoic acid methyl ester (85 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (5%) to give the title compound (70 mg)

LC/MS t=4.02 min, [MH⁺] 500, 502.

### b) 3-{2-[5-Chloro-2-(benzyloxy)-phenyl]-5-methyl-pyrrol-1-yl}-5-trifluoromethyl-benzoic acid

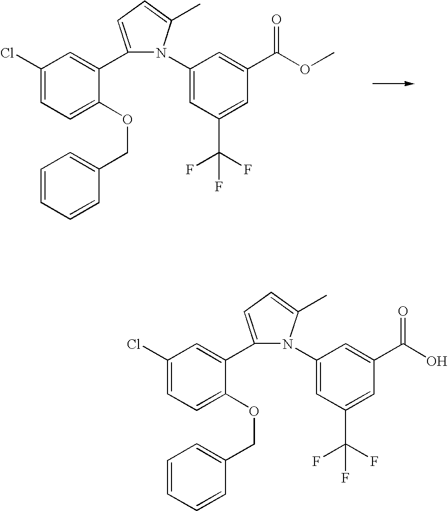

3-{2-[5-Chloro-2-(benzyloxy)-phenyl]-5-methyl-pyrrol-1-yl}-5-trifluoromethyl-benzoic acid methyl ester (105 mg) was heated at reflux in a mixture of EtOH (2.0 mL) and 2M NaOH (1.0 mL) for 2.5 hours. The mixture was diluted with DCM and 2M HCl and filtered through a hydrophobic frit, fitted with a plug of Na₂SO₄, and evaporated to give the title compound.

LC/MS t=4.32 min, [MH⁺] 486, 488; [MH⁻] 484, 486.

### Example 345

### 3-{2-[5-Chloro-2-(benzyloxy)-phenyl]-5-methyl-pyrrol-1-yl}-6-difluoromethoxy-benzoic acid

### a) 3-{2-[5-Chloro-2-(benzyloxy)-phenyl]-5-methyl-pyrrol-1-yl}-6-difluoromethoxy-benzoic acid methyl ester

1-[5-Chloro-2-benzyloxy)-phenyl]-pentane-1,4-dione (112 mg), 5-amino-2-difluoromethoxy-benzoic acid methyl ester (126 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (5-10%) to give the title compound (121 mg)

LC/MS t=4.11 min, [MH⁺] 498, 500

### b) 3-{2-[5-Chloro-2-(benzyloxy)-phenyl]-5-methyl-pyrrol-1-yl}-6-difluoromethoxy-benzoic acid

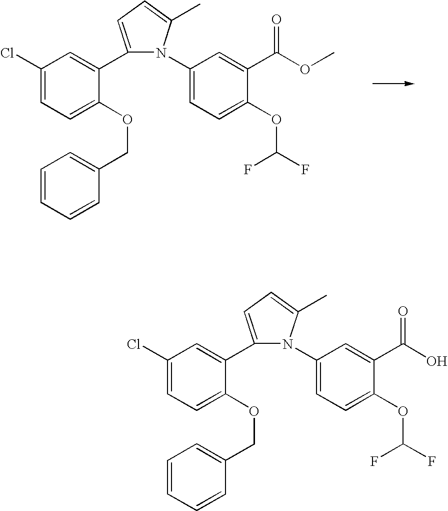

3-{2-[5-Chloro-2-(benzyloxy)-phenyl]-5-methyl-pyrrol-1-yl}-6-difluoromethoxy-benzoic acid methyl ester (117 mg) heated at reflux in a mixture of EtOH (3.0 mL) and 2M NaOH (1.5 mL) for 2 hours. The mixture was diluted with EtOAc and washed with 2M HCl, dried (Na₂SO₄), filtered and evaporated to give the title compound.

¹H NMR (400 MHz, d6-DMSO) 2.15 (3H, s), 4.71 (2H, s), 6.14 (1H, d, J=3 Hz), 6.30 (1H, d, J=3 Hz), 6.58 (1H, t, J=74 Hz), 6.60 (1H, d, J=9 Hz), 7.00-7.15 (5H, m), 7.21-7.34 (4H, m's excess), 7.65-7.69 (1H, m).

LC/MS t=3.75 min, [MH⁺] 484, 486; [MH⁻] 482, 484.

### Example 346

### 3-{2-[5-Chloro-2-benzyloxy)-phenyl]-5-methyl-pyrrol-1-yl}-5-(2-oxo-piperidin-1-yl)-benzoic acid

### a) 3-{2-[5-Chloro-2-(benzyloxy)-phenyl]-5-methyl-pyrrol-1-yl}-5-(2-oxo-piperidin-1-yl)-benzoic acid methyl ester

1-[5-Chloro-2-benzyloxy)-phenyl]-pentane-1,4-dione (111 mg), 3-amino-5-(2-oxo-piperidin-1-yl)-benzoic acid methyl ester (1 12 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (30-80%) to give the title compound (149 mg)

LC/MS t=3.87 min, [MH⁺] 529, 531

### b) 3-{2-[5-Chloro-2-benzyloxy)-phenyl]-5-methyl-pyrrol-1-yl}-5-(2-oxo-piperidin-1-yl)-benzoic acid

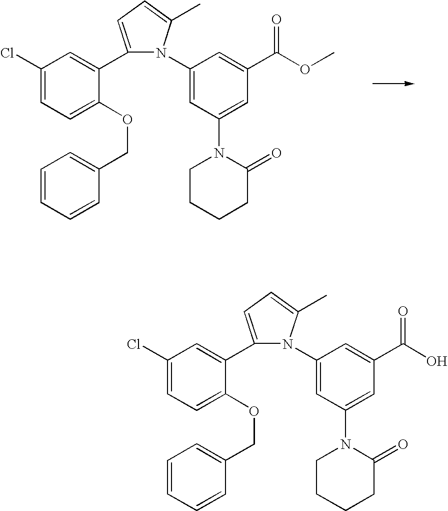

3-{2-[5-Chloro-2-(benzyloxy)-phenyl]-5-methyl-pyrrol-1-yl}-5-(2-oxo-piperidin-1-yl)-benzoic acid methyl ester (144 mg) was heated at reflux in a mixture of EtOH (3.0 mL) and 2M NaOH (1.5 mL) for 2 hours. The mixture was diluted with DCM and 2M HCl and filtered through a hydrophobic frit, fitted with a plug of Na₂SO₄, and evaporated to give the title compound (134 mg).

LC/MS t=3.55 min, [MH⁺] 515, 517; [MH⁻] 513, 515.

### Example 347

### 3-{2-[5-Chloro-2-(benzyloxy)-phenyl]-5-methyl-pyrrol-1-yl}-6-acetylamino-benzoic acid

### a) 3-{2-[5-Chloro-2-benzyloxy)-phenyl]-5-methyl-pyrrol-1-yl}-6-acetylamino-benzoic acid methyl ester

1-[5-Chloro-2-benzyloxy)-phenyl]-pentane-1,4-dione (111 mg), 2-acetylamino-5-amino-benzoic acid methyl ester (97 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (5-30%) to give the title compound (124 mg)

LC/MS t=4.03 min, [MH⁺] 489, 491

### b) 3-{2-[5-Chloro-2-benzyloxy)-phenyl]-5-methyl-pyrrol-1-yl}-6-acetylamino-benzoic acid

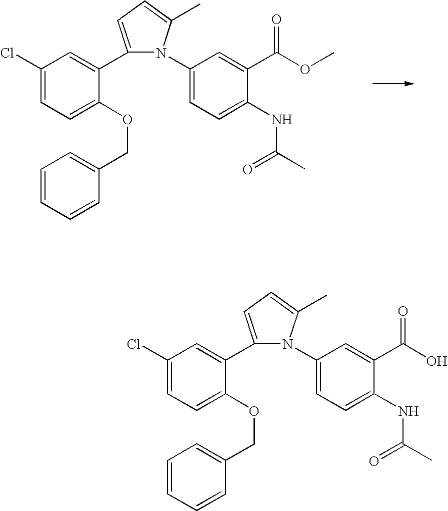

3-{2-[5-Chloro-2-(benzyloxy)-phenyl]-5-methyl-pyrrol-1-yl}-6-acetylamino-benzoic acid methyl ester (148 mg) heated at 120° C. in a microwave in a mixture of EtOH (3.0 mL) and 2M NaOH (1.0 mL) for 3 minutes. The mixture was diluted with DCM and 2M HCl and filtered through a hydrophobic frit, fitted with a plug of Na₂SO₄, and evaporated. The residue was purified by MDAP to give the title compound (46 mg).

¹H NMR (400 MHz, d6DMSO) 2.13 (3H, s), 2.24 (3H, s), 4.76 (2H, s), 6.12 (1H, d, J=3 Hz), 6.29 (1H, d, J=4 Hz), 6.59 (1H, d, J=9 Hz), 7.02-7.12 (3H, m), 7.16 (1H, dd, J=3 Hz, J=9 Hz), 7.22-7.33 (5H, m's excess), 7.75 (1H, d, J=2 Hz), 8.60 (1H, d, J=9 Hz), 10.85 (1H, s).

LC/MS t=4.15 min, [MH⁺] 475, 477; [MH⁻] 473, 475.

### Example 348

### 3-{2-[5-Chloro-2-(benzyloxy)-phenyl]-5-methyl-pyrrol-1-yl}-5-amino-6-methyl-benzoic acid

### a) 3-{2-[5-Chloro-2-(benzyloxy)-phenyl]-5-methyl-pyrrol-1-yl}-5-amino-6-methyl-benzoic acid methyl ester

1-[5-Chloro-2-benzyloxy)-phenyl]-pentane-1,4-dione (203 mg), 3,5-diamino-2-methyl-benzoic acid methyl ester (1 54 mg) and pTSA (cat) were heated in toluene (2.6 mL) at reflux for 4 hrs. The mixture was diluted with EtOAc and washed sequentially with saturated NH₄Cl and saturated NaHCO₃, dried (Na₂SO₄), filtered and concentrated. The residue was purified on silica gel with iso-hexane containing EtOAc (30-50%) to give the title compound (106 mg)

LC/MS t=3.94 min, [MH⁺] 461, 463

### b) 3-{2-[5-Chloro-2-(benzyloxy)-phenyl]-5-methyl-pyrrol-1-yl}-6-amino-6-methyl-benzoic acid

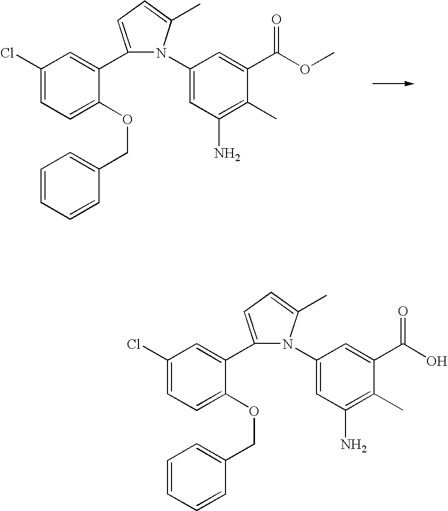

3-{2-[5-Chloro-2-(benzyloxy)-phenyl]-5-methyl-pyrrol-1-yl}-5-amino-6-methyl-benzoic acid methyl ester (35 mg) was heated at reflux in a mixture of EtOH (0.7 mL) and 2M NaOH (0.35 mL) for 2.5 hours. The mixture was diluted with EtOAc and washed with 2M HCl, dried (Na₂SO₄), filtered and evaporated to give the title compound.

¹H NMR (400 MHz, d6-DMSO) 2.04 (3H, s), 2.21 (3H, s), 4.95 (2H, s), 5.99 (1H, d, J=3 Hz), 6.20 (1H, d, J=3 Hz), 6.55 (1H, d, J=2 Hz), 6.68 (1H, d, J=2 Hz), 6.87 (1H, d, J=9 Hz), 7.02 (1H, d, J=3 Hz), 7.14 (1H, dd, J=3 Hz, J=9 Hz), 7.18-7.38 (5H, m).

LC/MS t=3.49 min, [MH⁺] 447, 449; [MH⁻] 445, 447.

### Example 349

### 3-{2-[5-Chloro-2-(benzyloxy)-phenyl]-5-methyl-pyrrol-1-yl}-6-fluoro-benzoic acid

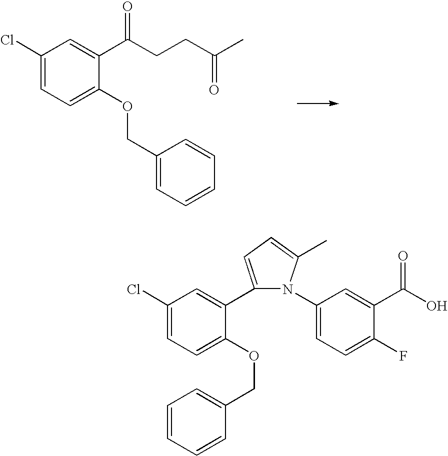

1-[5-Chloro-2-benzyloxy)-phenyl]-pentane-1,4-dione (100 mg), 5-amino-2-fluoro-benzoic acid and powdered 4 A molecular sieves (300 mg) were heated in toluene (0.2M solution) at reflux for 24 hrs. The mixture was diluted with EtOAc filtered and evaporated. The residue was purified by MDAP, to give the title compound (54 mg).

¹H NMR (400 MHz, CDCl₃) 2.14 (3H, s), 4.75 (2H, s), 6.13 (1H, dd, J=1 Hz, J=3 Hz), 6.29 (1H, J=3 Hz), 6.61 (1H, d, J=9 Hz), 6.94-7.12 (5H, m), 7.23-7.34 (4H, m's excess), 7.64-7.70 (1H, m).

LC/MS t=3.76 min, [MH⁺] 436, 438; [MH⁻] 434

### Example 350

### 3-{2-[5-Chloro-2-(benzyloxy)-phenyl]-5-methyl-pyrrol-1-yl}-5-acetylamino-benzoic acid

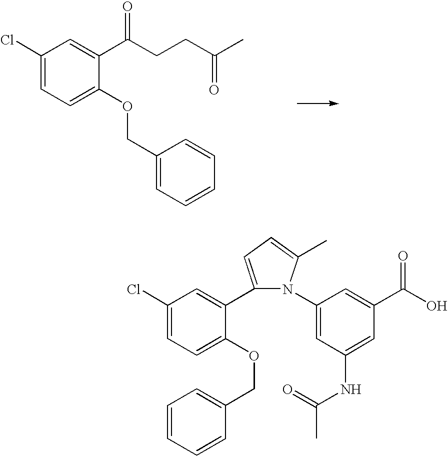

1-[5-Chloro-2-benzyloxy)-phenyl]-pentane-1,4-dione (100 mg), 3-acetylamino-5-amino-benzoic acid (60 mg) and powdered 4A molecular sieves (300 mg) were heated in toluene (0.2M solution) at reflux for 24 hrs. The mixture was diluted with EtOAc filtered and evaporated. The residue was purified by MDAP, to give the title compound (77 mg).

¹H NMR (400 MHz, CDCl₃) 2.12 (3H, s), 2.15 (3H, s), 4.75 (2H, s), 6.13 (1H, J=3 Hz), 6.30 (1H, J=3 Hz), 6.58 (1H, d, J=9 Hz), 6.94-7.13 (4H, m), 7.23-7.37 (4H, m's excess), 7.44 (1H, s), 8.07 (1H, s).

LC/MS t=3.46 min, [MH⁺] 475, 477; [MH⁻] 473, 475.

### Example 351

### 3-{2-[5-Chloro-2-(benzyloxy)-phenyl]-5-methyl-pyrrol-1-yl}-1-napthoic acid

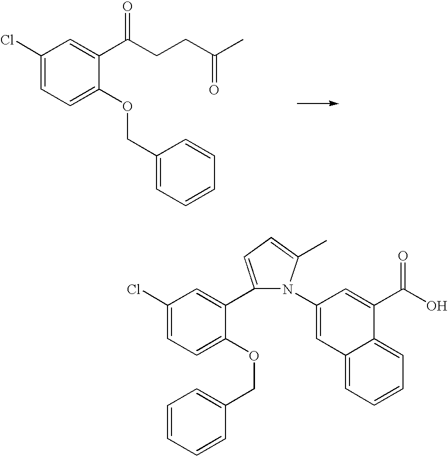

1-[5-Chloro-2-benzyloxy)-phenyl]-pentane-1,4-dione (100 mg), 3-amino-naphthalene-1-carboxylic acid (71 mg) and powdered 4A molecular sieves (300 mg) were heated in toluene (0.2M solution) at reflux for 24 hrs. The mixture was diluted with EtOAc filtered and evaporated. The residue was purified by MDAP, to give the title compound (1 13 mg).

¹H NMR (400 MHz, CDCl₃) 2.20 (3H, s), 4.65 (2H, s), 6.19 (1H, J=3 Hz), 6.35 (1H, J=3 Hz), 6.47 (1H, d, J=9 Hz), 6.88-6.96 (2H, m), 7.01 (1H, dd, J=2 Hz, J=9 Hz), 7.12-7.23 (3H, m), 7.38 (1H, d, J=2 Hz), 7.48-7.56 (1H, m), 7.60 (1H, d, J=1 Hz), 7.61-7.70 (2H, m), 8.10 (1H, d, J=2 Hz), 9.03 (1H, d, J=9 Hz).

LC/MS t=4.20 min, [MH⁺] 468, 470; [MH⁻] 466, 468.

### Example 352

### 3-{2-[5-Chloro-2-(benzyloxy)-phenyl]-5-methyl-pyrrol-1-yl}-4-fluoro-benzoic acid

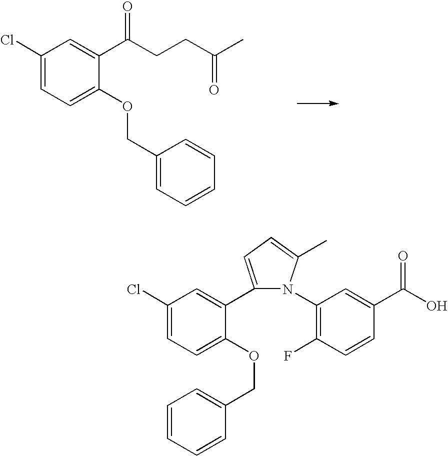

1-[5-Chloro-2-benzyloxy)-phenyl]-pentane-1,4-dione (100 mg), 3-amino-4-fluoro-benzoic acid (60 mg) and powdered 4A molecular sieves (300 mg) were heated in toluene (0.2M solution) at reflux for 24 hrs. The mixture was diluted with EtOAc filtered and evaporated. The residue was purified by MDAP, to give the title compound (63 mg).

¹H NMR (400 MHz, CDCl₃) 2.11 (3H, s), 4.84 (2H, s), 6.16 (1H, dd, J=1 Hz, J=3 Hz), 6.34 (1H, J=3 Hz), 6.56 (1H, d, J=9 Hz), 7.02 (1H, dd, J=3 Hz, J=9 Hz), 7.04-7.15 (2H, m), 7.22 (1H, d, J=2 Hz), 7.23-7.32 (4H, m's excess), 7.81 (1H, dd, J=2 Hz, J=7 Hz), 7.98-8.04 (1H, m).

LC/MS t=3.99 min, [MH⁺] 436, 438; [MH⁻] 434, 436.

### Example 353

### 3-{2-[5-Chloro-2-(benzyloxy)-phenyl]-5-methyl-pyrrol-1-yl}-5-methyl-benzoic acid

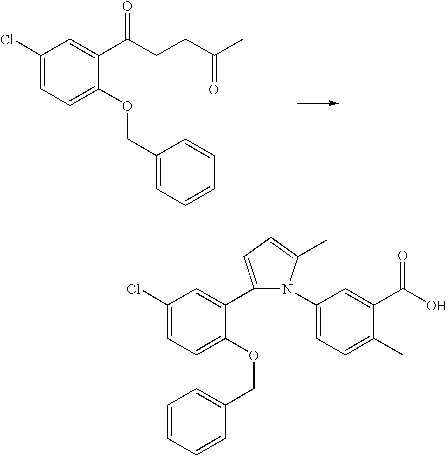

1-[5-Chloro-2-(benzyloxy)-phenyl]-pentane-1,4-dione (111 mg), 5-amino-2-methyl-benzoic acid (61 mg) and pTSA (cat) were heated in NMP (4.5 mL) at 150° C. for 10 minutes. The mixture was diluted with Et₂O washed with 2M HCl, dried (Na₂SO₄), filtered and evaporated. The residue was purified by MDAP, to give the title compound (89 mg).

¹H NMR (400 MHz, CDCl₃) 2.15 (3H, s), 2.62 (3H, s), 4.77 (2H, s), 6.13 (1H, d, J=3 Hz), 6.30 (1H, J=3 Hz), 6.57 (1H, d, J=9 Hz), 7.00-7.13 (5H, m), 7.20-7.30 (4H, m's excess), 7.77 (1H, m).

LC/MS t=4.04 min, [MH⁺] 432, 434; [MH⁻] 430, 432.

### Example 354

### 3-{2-[2-(2-Chloro-4-fluoro-benzyloxy)-phenyl]-5-methyl-pyrrol-1-yl}-N-(3,5-dimethyl-isoxazole-4-sulfonyl)-benzenamide

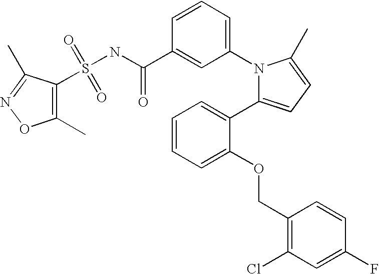

Prepared in the same way as 3-{2-[2-(4-fluoro-benzyloxy)-phenyl]-5-methyl-pyrrol-1-yl}-N-(3,5-dimethyl-isoxazole-4-sulfonyl)-benzenamide.

LC/MS t=4.55 min, [MH⁺] 594, 596; [MH⁻] 592, 594.

### Example 355

### 3-{2-[5-Chloro-2-(benzyloxy)-phenyl]-5-methyl-pyrrol-1-yl}-5-amino-benzoic acid

### a) 3-{2-[5-Chloro-2-(benzyloxy)-phenyl]-5-methyl-pyrrol-1-yl}5-amino-benzoic acid methyl ester

1-[5-Chloro-2-benzyloxy)-phenyl]-pentane-1,4dione (824 mg, 2.60 mmol), 3,5-diamino-benzoic acid methyl ester (550 mg, 3.31 mmol) and pTSA (cat) were heated at reflux in toluene (1 0 mL) for 2.5 hrs. The mixture was allowed to stand overnight, diluted with EtOAc, and washed sequentially with saturated NH₄Cl and saturated NaHCO₃, dried (Na₂SO₄), filtered and evaporated. The residue was purified by chromatography on silica gel with iso-hexane containing EtOAc (240%) as eluant, to give the title compound (946 mg).

LC/MS t=3.89 min, [MH⁺] 447, 449.

### b) 3-{2-[5-Chloro-2-(benzyloxy)-phenyl]-5-methyl-pyrrol-1-yl}-5-amino-benzoic acid

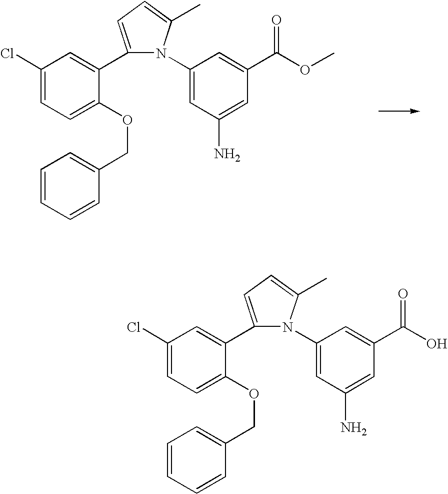

3-{2-[5-Chloro-2-(benzyloxy)-phenyl]-5-methyl-pyrrol-1-yl}-5amino-benzoic acid methyl ester (67 mg) was heated at reflux in a mixture of EtOH (1.4 mL) and 2M NaOH (0.7 mL) for 2.5 hours. The mixture was diluted with EtOAc and washed with 2M HCl, dried (Na₂SO₄), filtered and evaporated to give the title compound.

¹H NMR (400 MHz, CDCl₃) 2.59 (3H, s), 5.46 (2H, s), 6.56 (1H, d, J=3 Hz), 6.78 (1H, d, J=3 Hz), 7.18 (1H, s), 7.39-7.48 (2H, m), 7.53 (1H, d, J=2 Hz), 7.68 (1H, dd, J=3 Hz, J=9 Hz), 7.71-7.90 (6H, m's excess).

LC/MS t=3.73 min, [MH⁺] 433, 435; [MH⁻] 431, 433.

### Example 356

### 3-{2-[5-Chloro-2-(benzyloxy)-phenyl]-5-methyl-pyrrol-1-yl}-5-methoxycarbonylamino-benzoic acid

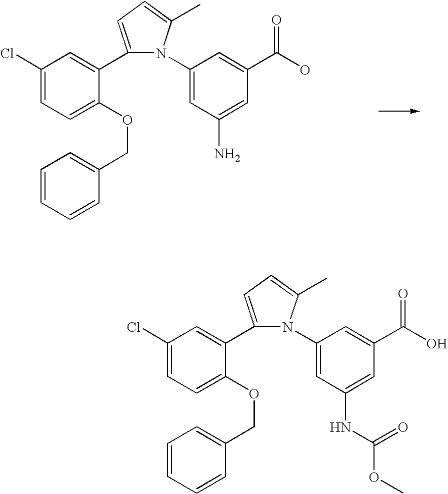

Methyl chloroformate was added dropwise to a solution of 3-{2-[5-chloro-2-(benzyloxy)-phenyl]-5-methyl-pyrrol-1-yl}-5-amino-benzoic acid and DMAP (cat) in DCM-pyridine (1:1, 2 mL). The mixture was stirred at room temperature for 2.5 hrs then allowed to stand to 6 days then diluted with DCM and washed sequentially with 2M HCl and saturated NaHCO₃, dried (Na₂SO₄), filtered and concentrated. The residue was purified by MDAP to give the title compound.

¹H NMR (400 MHz, CDCl₃) 2.15 (3H, s), 3.76 (3H, s),4.74 (2H, s), 6.12 (1H, d, H=3 Hz), 6.29 (1H, d, J=3 Hz), 6.51 (1H, br), 6.58 (1H, d, J=9 Hz), 6.70 (1H, s), 7.02-7.12 (3H, m), 7.18-7.33 (5H, m's excess), 7.41 (1H, s), 7.95 (1H, s).

LC/MS t=3.82 min [MH⁺] 491, 493; [MH⁻] 489, 491.

### Example 357

### 3-{2-[5-Chloro-2-(benzyloxy)-phenyl]-5-methyl-pyrrol-1-yl}-6-hydroxy-benzoic acid

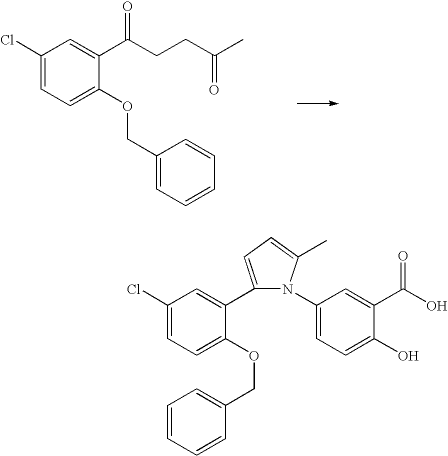

1-[5-Chloro-2-benzyloxy)-phenyl]-pentane-1,4-dione (103 mg, 0.33 mmol), 2-hydroxy-5-amino-benzoic acid (66 mg, 0.43 mmol) and pTSA (cat) were heated in NMP (1.5 mL) at 150° C. in a microwave for 10 minutes. The mixture was diluted with Et₂O and washed with 2M HCl, dried (Na₂SO₄), filtered and concentrated. The residue was purified on silica gel with iso-hexane containing EtOAc (50%) to give the title compound (56 mg)

¹H NMR (400 MHz, CDCl₃) 2.09 (3H, br s), 4.78 (2H, br s), 6.10 (br s), 6.27 (1H, br d, J=2 Hz), 6.57 (1H, br d, J=9 Hz), 6.79 (1H, br s), 6.93-7.13 (4H, m), 7.16-7.33 (4H, m's excess), 7.55 (1H, br s).

LC/MS t=4.59 min, [MH⁺] 434, 436; [MH⁻] 432, 434

### Example 358

### 6-{2-[5-Chloro-2-(benzyloxy)-phenyl]-5-methyl-pyrrol-1-yl}-1H-indole-4-carboxylic acid

### a) 6-{2-[5-Chloro-2-(benzyloxy)-phenyl]-5-methyl-pyrrol-1-yl}-1H-indole-4-carboxylic acid methyl ester

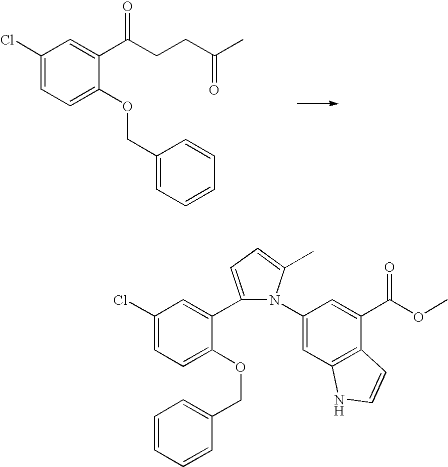

1-[5-chloro-2-benzyloxy)-phenyl]-pentane-1,4-dione, 6-amino-1H-indolecarboxylic acid methyl ester (prepared from 3,5-dinitro-o-toluic acid methyl ester using the Batcho-Leimgruber method as described by Wender et al., Proceedings of the National Academy of Sciences of USA, 1986, 83 (12), 4214-4218) (1 equivalent) and pTSA (cat) were heated in NMP (1.5 mL) at 150° C. in a microwave for 10 minutes. The mixture was diluted with EtOAc and washed sequentially with 2M HCl and saturated NaHCO₃, dried (Na₂SO₄), filtered and concentrated. The residue was purified on silica gel with iso-hexane containing EtOAc (10-20%) to give the title compound.

LC/MS t=4.02 min, [MH⁺] 471, 473; [MH⁻] 469, 471.

### b) 6-{2-[5-Chloro-2-(benzyloxy)-phenyl]-5-methyl-pyrrol-1-yl}-1H-indole-4carboxylic acid

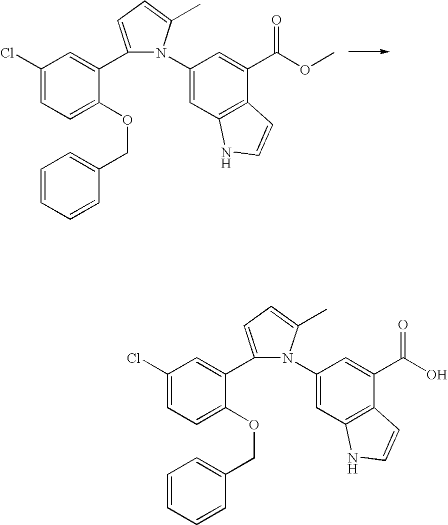

6-{2-[5-Chloro-2-(benzyloxy)-phenyl]-5-methyl-pyrrol-1-yl}-1H-indole-4-carboxylic acid methyl ester, was heated at 100° C. in a microwave in a mixture of EtOH (2 mL) and 2M NaOH (1 mL) for 2 minutes. The mixture was diluted with 2M HCl, and the resultant precipitate was collected by filtration to give the title compound.

LC/MS t=3.77 min, [MH⁺] 457, 459; [MH⁻] 455, 457.

¹H NMR (400 MHz, d6-DMSO) 2.05 (3H, s), 4.89 (2H, s), 6.05 (1H, d, J=3 Hz), 6.24 (1H, d, J=3 Hz), 6.93-6.99 (1H, m), 7.06-7.15 (4H, m's excess), 7.22-7.29 (3H, m's excess), 7.36 (1H, s), 7.45 (1H, d, J=2 Hz), 7.53 (1H, d, J=3 Hz), 11.41 (1H, s), 12.59 (1H, s).

### Example 359

### 3-{2-[5-Chloro-2-benzyloxy)-phenyl]-5-methyl-pyrrol-1-yl}-5-methanesulfonylamino-benzoic acid

### a) 3-{2-[5-Chloro-2-(benzyloxy)-phenyl]-5-methyl-pyrrol-1-yl}-5-methanesulfonylamino-benzoic acid methyl ester

Methanesulfonyl chloride (0.1 mL, 1.29 mmol) was added dropwise to a solution of 3-{2-[5-chloro-2-(benzyloxy)-phenyl]-5-methyl-pyrrol-1-yl}-5-amino-benzoic acid methyl ester (104 mg, 0.23 mmol) and DMAP (cat in DCM-pyridine (1:1, 2 mL). The mixture was stirred at room temperature for 2.5 hrs then allowed to stand to 6 days then diluted with DCM and washed sequentially with 2M HCl and saturated NaHCO₃, dried (Na₂SO₄), filtered and concentrated. The residue was purified on silica gel with iso-hexane containing EtOAc (20-30%) to give the title compound (99 mg, 81%).

LC/MS t=3.63 min [MH⁺] 445

### b) 3-{2-[5-Chloro-2-(benzyloxy)-phenyl]-5-methyl-pyrrol-1-yl}-5-methanesulfonylamino-benzoic acid

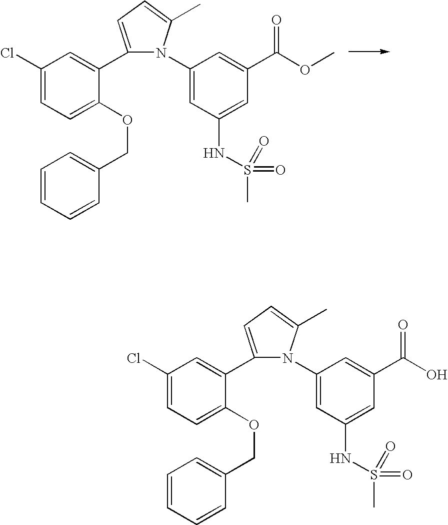

3-{2-[5-Chloro-2-(benzyloxy)-phenyl]-5-methyl-pyrrol-1-yl}-5-methanesulfonylamino-benzoic acid methyl ester (99 mg) was heated at 100° C. in a mixture of EtOH (3 mL) and 2M NaOH (1 mL) in a microwave for 2 minutes. The mixture was diluted with DCM and 2M HCl and filtered through a hydrophobic frit, fitted with a plug of Na₂SO₄, and evaporated to give the title compound (90 mg).

¹H NMR (400 MHz, CDCl₃) 2.19 (3H, s), 2.68 (3H, s), 4.79 (2H, s), 6.15 (1H, d, H=3 Hz), 6.33 (1H, d, J=3 Hz), 6.62 (1H, d, J=9 Hz), 6.70 (1H, s), 7.01-7.16 (4H, m), 7.23 (1H, d, J=2 Hz), 7.25-7.34 (2H, m's excess), 7.60 (1H, s), 7.70 (1H, d, J=5 Hz).

LC/MS t=3.73 min [MH⁺] 511, 513; [MH⁻] 509, 511.

### Example 360

### 3-{2-[5-Chloro-2-(benzyloxy)-phenyl]-5-methyl-pyrrol-1-yl}-5-(2-oxo-pyrrolidin-1-yl)-benzoic acid

### a) 3-{2-[5-Chloro-2-(benzyloxy)-phenyl]-5-methyl-pyrrol-1-yl}-5-(2-oxo-pyrrolidin-1-yl)-benzoic acid methyl ester

1-[5-Chloro-2-(benzyloxy)-phenyl]-pentane-1,4-dione (114 mg), 3-amino-5-(2-oxo-pyrrolidin-1yl)-benzoic acid methyl ester (103 mg) and pTSA (cat) were heated in NMP (1.5 mL) at 150° C. in a microwave for 10 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (30 -50%) to give the title compound (108 mg, 65%).

LC/MS t=3.96 min, [MH⁺] 515, 517.

### b) 3-{2-[5-Chloro-2-(benzyloxy)-phenyl]-5-methyl-pyrrol-1-yl}-5-(2-oxo-pyrrolidin-1-yl)-benzoic acid

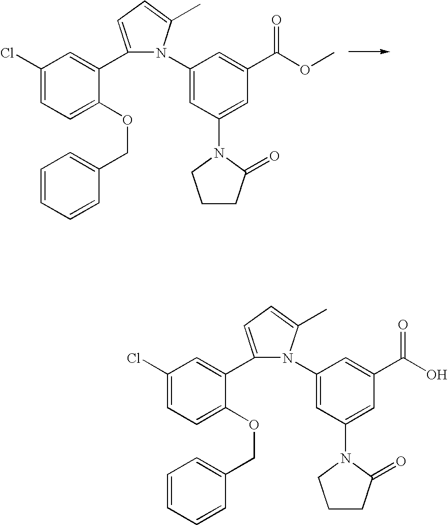

3-{2-[5-Chloro-2-(benzyloxy)-phenyl]-5-methyl-pyrrol-1-yl}-5-(2-oxo-pyrrolidin-1-yl)-benzoic acid methyl ester (108 mg) was heated at reflux in a mixture of EtOH (2 mL) and 2M NaOH (1 mL) for 1.5 hours. The mixture was diluted with DCM and 2M HCl and filtered through a hydrophobic frit, fitted with a plug of Na₂SO₄, and evaporated to give the title compound.

¹H NMR (400 MHz, d6DMSO) 1.94-2.04 (2H, m), 2.44-2.55 (2H, m's excess), 3.55-3.65 (2H, m), 4.81 (2H, s), 6.08 (1H, dd, J=0.5 Hz, J=3 Hz), 6.24 (1H, d, J=3 Hz), 6.85 (1H, d, J=9 Hz), 7.05-7.12 (2H, m), 7.16-7.34 (6H, m), 7.56 (1H, t, J=2 Hz), 8.20 (1H, t, J=1Hz), 13.00 (1H, br s).

LC/MS t=3.78 min, [MH⁺] 501, 503; [MH⁻] 499, 501.

### Example 361

### 3-{2-[5-Chloro-2-(benzyloxy)-phenyl]-5-methyl-pyrrol-1-yl}-5-(1,1-dioxo-1I⁶-isothiazolidin-2-yl)-benzoic acid

### a) 3-{2-[5-Chloro-2-(benzyloxy)-phenyl]-5-methyl-pyrrol-1-yl}-5-(1,1-dioxo-1I⁶-isothiazolidin-2-yl)-benzoic acid methyl ester

1-[5-Chloro-2-(benzyloxy)-phenyl]-pentane-1,4-dione (110 mg), 3-amino-5-(1,1-dioxo-1I⁶-isothiazolidin-2-yl)-benzoic acid methyl ester (1 32 mg) and pTSA (cat) were heated in NMP (1.5 mL) at 150° C. in a microwave for 30 minutes. The mixture was diluted with Et₂O and washed sequentially with 2M HCl and saturated NaHCO₃, dried (Na₂SO₄), filtered and concentrated. The residue was purified on silica gel with iso-hexane containing EtOAc (30-50%) to give the title compound (113 mg).

LC/MS t=3.93 min [MH⁺] 551, 553.

### b) 3-{2-[5-Chloro-2-(benzyloxy)-phenyl]-5-methyl-pyrrol-1-yl}-5-(1,1-dioxo-1I⁶-isothiazolidin-2-yl)-benzoic acid

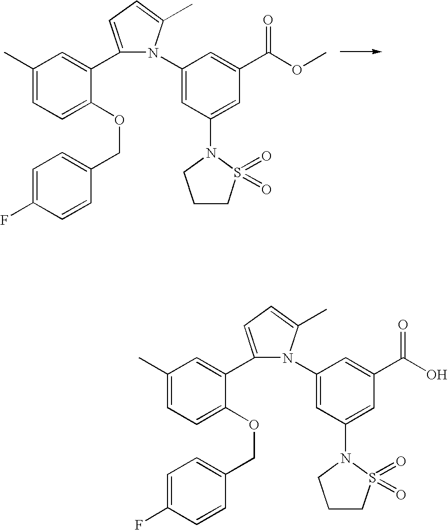

3-{2-[5-Chloro-2-(benzyloxy)-phenyl]-5-methyl-pyrrol-1-yl}-5-(1,1-dioxo-1I⁶-isothiazolidin-2-yl)-benzoic acid methyl ester (1 12 mg) was heated at reflux in a mixture of EtOH (2 mL) and 2M NaOH (1 mL) for 1.5 hours. The mixture was diluted with DCM and 2M HCl and filtered through a hydrophobic frit, fitted with a plug of Na₂SO₄, and evaporated to give the title compound.

¹H NMR (400 MHz, d6-DMSO) 2.30-2.40 (2H, m), 3.48-3.59 (4H, m), 4.84 (2H, s), 6.19 (1H, d, J=3 Hz), 6.24 (1H, d, J=3 Hz), 6.85 (1H, d, J=9 Hz), 7.06 (1H, t, J=2 Hz), 7.08-7.35 (8H, m), 7.68-7.72 (1H, m).

LC/MS t=3.73 min, [MH⁺] 537, 539; [MH⁻] 535, 537.

### Example 362

### 3-{2-[5-Chloro-2-(tetrahydro-pyran-4-ylmethoxy)-phenyl]-5-methyl-pyrrol-1-yl}-benzoic acid

### a) Toluene-4-sulfonic acid tetrahydro-pyran-4-yl-methyl ester

Tetrahydropyran-4-yl-carboxylic acid (207 mg, 1.59 mmol) was dissolved in THF (3.2 mL). 1M borane-THF (3.2 mL) was added and the mixture was stirred at room temperature for 6 hours, after which time water was added. The mixture was extracted twice with EtOAc. The combined extracts were dried (Na₂SO₄), filtered and concentrated. The residue was dissolved in DCM-pyridine (1:1, 3.2 mL). Tosyl chloride (327 mg, 1.71 mmol) was added to this solution. Stirring was continued overnight, after which time the mixture was diluted with DCM and washed sequentially with 2M HCl and saturated NaHCO₃, dried (Na₂SO₄), filtered and concentrated. The residue was purified on silica gel with iso-hexane containing EtOAc (20%), as eluant, to give the title compound (66 mg).

Rf 0.49 (50% EtOAc in iso-hexane).

¹H NMR (400 MHz, CDCl₃) 1.17 (2H, m's excess), 1.51-1.66 (2H, m's excess), 1.86-2.01 (1H, m), 2.47 (3H, s), 3.34 (2H, t, J=12 Hz), 3.86 (2H, d, J=6 Hz), 3.95-4.00 (2H, m), 7.35 (2H, d, J=8 Hz), 7.78 (2H, d, J=8 Hz).

### b) 3-{2-[5-Chloro-2-(tetrahydro-pyran-4-ylmethoxy)-phenyl]-5-methyl-pyrrol-1-yl}-benzoic acid ethyl ester

3-{2-[5-Chloro-2-(hydroxy)-phenyl]-5-methyl-pyrrol-1-yl}-benzoic acid ethyl ester (27 mg) was heated in DMF at 60° C. with potassium carbonate (25 mg) and toluene-4-sulfonic acid tetrahydro-pyran-4-yl-methyl ester (21 mg) for 24 hours. The mixture was then diluted with Et₂O and water. The organic phase was separated, dried (Na₂SO₄), filtered and concentrated to give the title compound (27 mg).

LC/MS t=3.98 min [MH⁺] 454, 456.

### c) 3-{2-[5-Chloro-2-(tetrahydro-pyran-4-ylmethoxy)-phenyl]-5-methyl-pyrrol-1-yl}-benzoic acid

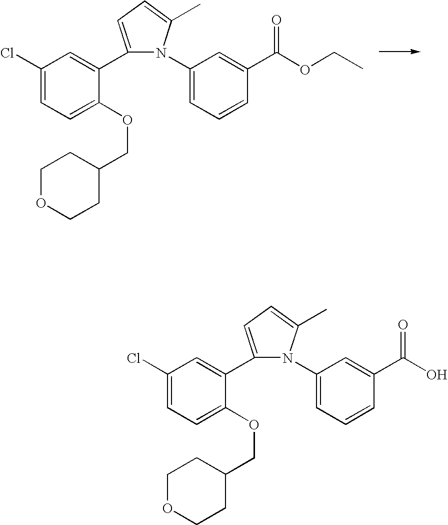

3-{2-[5-Chloro-2-(tetrahydro-pyran-4-ylmethoxy)-phenyl]-5-methyl-pyrrol-1-yl}-benzoic acid ethyl ester (27 mg) was heated at reflux in a mixture of EtOH (0.6 mL) and 2M NaOH (0.3 mL) for 1.5 hours. The mixture was diluted with EtOAc and washed with 2M HCl, dried (Na₂SO₄), filtered and evaporated. The residue was purified by MDAP to give the title compound (1 3 mg).

LC/MS t=3.72 min [MH⁺] 426, 428; [MH⁻] 424, 426.

### Example 363

### 3-{2-[5-Chloro-2-(tetrahydro-furan-3-ylmethoxy)-phenyl]-5-methyl-pyrrol-1-yl}-benzoic acid

### a) 3-{2-[5-Chloro-2-(tetrahydro-furan-3-ylmethoxy)-phenyl]-5-methyl-pyrrol-1-yl}-benzoic acid ethyl ester

3-{2-[5-Chloro-2-(hydroxy)-phenyl]-5-methyl-pyrrol-1-yl}-benzoic acid ethyl ester (1 95 mg) was heated in DMF (1.2 mL) at 60° C. with potassium carbonate (166 mg) and tetrahydrofurfuryl bromide (1 72 mg) for 24 hours. Further tetrahydrofurfuryl bromide (0.400g) was added and heating continued for 20 hours. The mixture was then diluted with Et₂O and water. The organic phase was separated, dried (Na₂SO₄), filtered and concentrated to give the title compound.

LC/MS t=3.95 min [MH⁺] 440, 442.

### b) 3-{2-[5-Chloro-2-(tetrahydro-furan-3-ylmethoxy)-phenyl]-5-methyl-pyrrol-1-yl}-benzoic acid

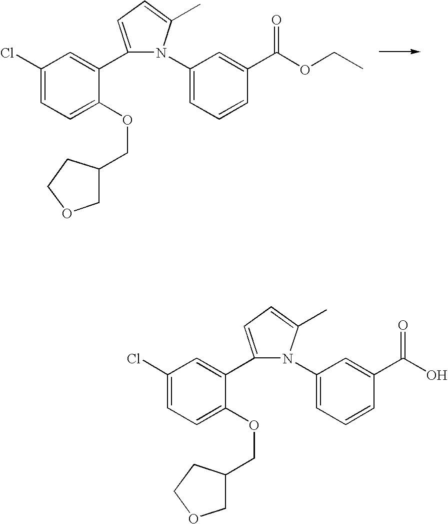

3-{2-[5-Chloro-2-(tetrahydro-furan-3-ylmethoxy)-phenyl]-5-methyl-pyrrol-1-yl}-benzoic acid ethyl ester (11 0 mg) was heated at reflux in a mixture of EtOH (2 mL) and 2M NaOH (1 mL) for 2 hours. The mixture was diluted with EtOAc and washed with 2M HCl, dried (Na₂SO₄), filtered and evaporated. The residue was purified by MDAP to give the title compound.

LC/MS t=3.68 min [MH⁺] 412, 414; [MH⁻] 410, 412.

### Example 364

### 3-{2-[5-Bromo-2-(5-methyl-isoxazol-3-yl-methoxy)-phenyl]-5methyl-pyrrol-1-yl}-benzoic acid

### a) 3-{2-[5-Bromo-2-(5-methyl-isoxazol-3-yl-methoxy)-phenyl]-5-methyl-pyrrol-1-yl}-benzoic acid ethyl ester

3-{2-[5-Bromo-2-(hydroxy)-phenyl]-5-methyl-pyrrol-1-yl}-benzoic acid ethyl ester (0.3 mmol) was heated in DMF (1.5 mL) at 60° C. with potassium carbonate (64 mg), 3-(chloromethyl)-5-methyl isoxazole (45 mg) and sodium iodide (cat) for 5.5 hours. The mixture was then diluted with Et₂O and water. The organic phase was separated, dried (Na₂SO₄), filtered and concentrated. The residue was chromatographed on silica gel with iso-hexane containing EtOAc (10%), as eluant, to give the title compound.

LC/MS t=4.01 min [MH⁺] 495, 497.

### b) 3-{2-[5-bromo-2-(5-methyl-isoxazol-3-yl-methoxy)-phenyl]-5-methyl-pyrrol-1-yl}-benzoic acid

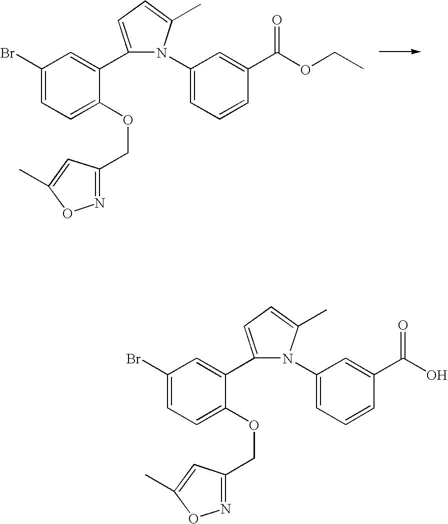

3-{2-[5-Bromo-2-(5-methyl-isoxazol-3-yl-methoxy)-phenyl]-5-methyl-pyrrol-1-yl}-benzoic acid ethyl ester was heated, at 100° C. in a microwave, in a mixture of EtOH and 2M NaOH for 2 minutes. The mixture was diluted with 2M HCl, and the resultant precipitate was collected by filtration to give the title compound.

LC/MS t=3.77 min [MH⁺] 457, 459; [MH⁻] 465, 467.

### Example 365

### 4-{2-[5-Chloro-2-(benzyloxy)-phenyl]-5-methyl-pyrrol-1-yl}-N-(1-phenyl-methanoyl)-benzenesulfonamide

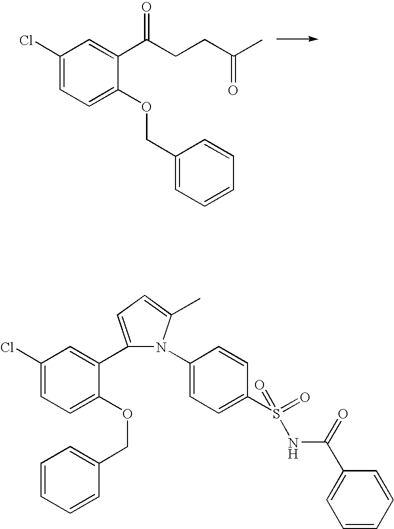

1-[5-Chloro-2-benzyloxy)-phenyl]-pentane-1,4-dione (252 mg, 0.80 mmol), sulfabenzamide (230 mg) and pTSA (cat) were heated at reflux in toluene (8 mL) for 4 hours. The mixture as diluted with EtOAc and washed with 2M HCl, dried (Na₂SO₄), filtered and concentrated. The residue was purified on silica gel with iso-hexane containing EtOAc (30-80%) to give the title compound.

LC/MS t=4.32 min, [MH⁺] 557, 559; [MH⁻] 555, 557.

### Example 366

### 4-{2-[5-Bromo-2-(benzyloxy)-phenyl]-5-methyl-pyrrol-1-yl}-N-(1-phenyl-methanoyl)-benzenesulfonamide

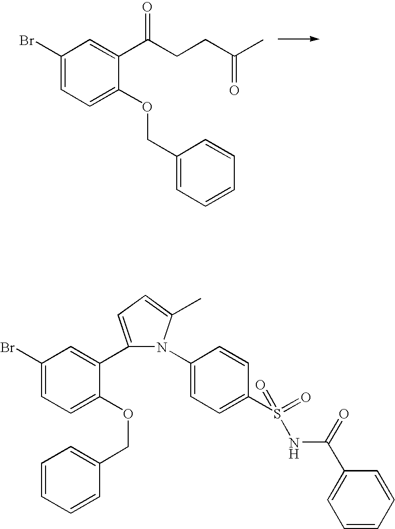

1-[5-Bromo-2-benzyloxy)-phenyl]-pentane-1,4-dione (263 mg, 0.73 mmol), sulfabenzamide (210 mg) and pTSA (cat) were heated at reflux in toluene (7.3 mL) for 3.5 hours. The mixture was diluted with EtOAc and washed with 2M HCl, dried (Na₂SO₄), filtered and concentrated. The residue was purified on silica gel with iso-hexane containing EtOAc (30-80%) to give the title compound.

LC/MS t=4.38 min, [MH⁺] 601, 603; [MH⁻] 599, 601.

### Example 367

### 4-{2-[5-Chloro-2-(benzyloxy)-phenyl]-5-methyl-pyrrol-1-yl}-N-(pyridin-2-ylmethyl)-benzamide

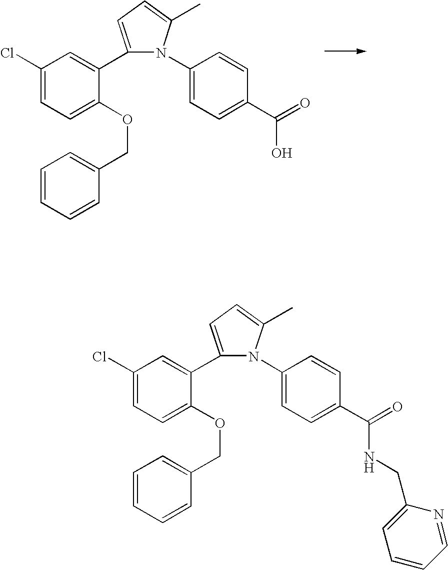

4-{2-[5-Chloro-2-(benzyloxy)-phenyl]-5-methyl-pyrrol-1-yl}-benzoic acid (24 mg) was dissolved in a mixture of MeCN (0.6 mL) and DCM (0.2 mL). EDC (15 mg) and HOBt (11 mg) were added and the mixture stirred for 5 minutes before 2-(aminomethyl)pyridine (12.5 μL) was added. The mixture was stirred overnight then diluted with EtOAc and washed sequentially with saturated NH₄Cl and saturated NaHCO₃, dried (Na₂SO₄), filtered and concentrated. The residue was purified on silica gel with EtOAc to give the title compound.

¹H NMR (400 MHz, CDCl₃) 2.17 (3H, s), 4.70 (2H, s), 4.75 (2H, d, J=5 Hz), 6.13 (1H, d, J=3 Hz), 6.30 (1H, d, J=3 Hz), 6.55 (1H, d, J=9 Hz), 6.95-7.18 (5H, m), 7.16-7.47 (5H, m's excess), 7.55-7.80 (4H, m), 8.57 (1H, d, J=5 Hz).

LC/MS t=3.79 min, [MH⁺] 508, 510.

### Example 368

### 4-{2-[S-Chloro-2-(benzyloxy)-phenyl]-5-methyl-pyrrol-1-yl}-N-(pyridin-3-ylmethyl)-benzamide

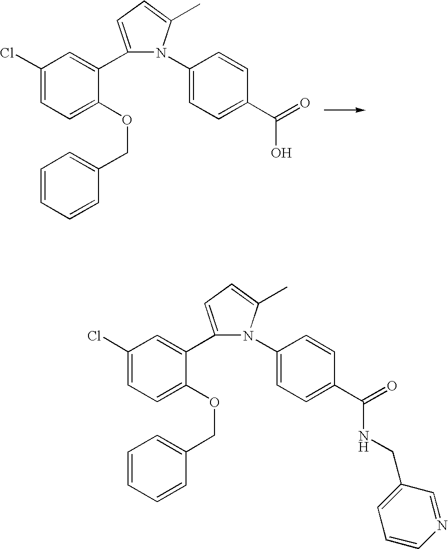

4-{2-[5-Chloro-2-(benzyloxy)-phenyl]-5-methyl-pyrrol-1-yl}-benzoic acid (24 mg) was dissolved in a mixture of MeCN (0.6 mL) and DCM (0.2 mL). EDC (15 mg) and HOBt (11 mg) were added and the mixture stirred for 5 minutes before 3-(aminomethyl)pyridine (12.5 μL) was added. The mixture was stirred overnight then diluted with EtOAc and washed sequentially with saturated NH₄Cl and saturated NaHCO₃, dried (Na₂SO₄), filtered and concentrated. The residue was purified by MDAP to give the title compound.

LC/MS t=3.64 min, [MH⁺] 508, 510, [MH⁻] 506, 508.

### Example 369

### 4-{2-[5-Chloro-2-(benzyloxy)-phenyl]-5-methyl-pyrrol-1-yl}-N-(pyridin-4-ylmethyl)-benzamide

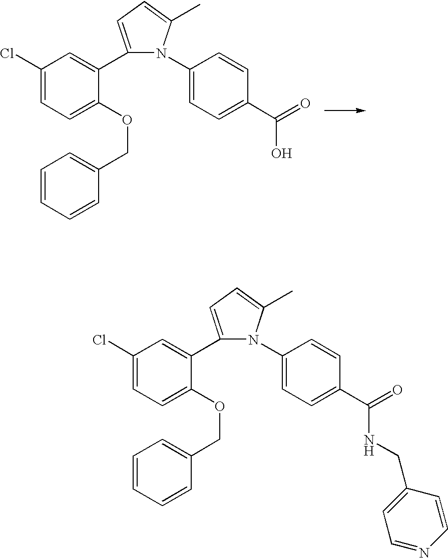

4-{2-[5-Chloro-2-(benzyloxy)-phenyl]-5-methyl-pyrrol-1-yl}-benzoic acid (24 mg) was dissolved in a mixture of MeCN (0.6 mL) and DCM (0.2 mL). EDC (15 mg) and HOBt (11 mg) were added and the mixture stirred for 5 minutes before 4-(aminomethyl)pyridine (12.5 μL) was added. The mixture was stirred overnight then diluted with EtOAc and washed sequentially with saturated NH₄Cl and saturated NaHCO₃, dried (Na₂SO₄), filtered and concentrated. The residue was purified by MDAP to give the title compound.

LC/MS t=3.51 min, [MH⁺] 508, 510, [MH⁻] 506, 508.

### Example 370

### 4-{2-[5-Chloro-2-(benzyloxy)-phenyl]-5-methyl-pyrrol-1-yl}-N-(benzyl)-benzamide

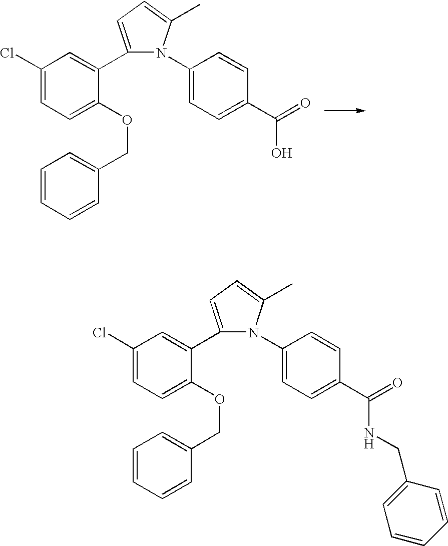

4-{2-[5-Chloro-2-(benzyloxy)-phenyl]-5-methyl-pyrrol-1-yl}-benzoic acid (24 mg) was dissolved in a mixture of MeCN (0.6 mL) and DCM (0.2 mL). EDC (15 mg) and HOBt (11 mg) were added and the mixture stirred for 5 minutes before benzylamine (12.5 μL) was added. The mixture was stirred overnight then diluted with EtOAc and washed sequentially with saturated NH₄Cl and saturated NaHCO₃, dried (Na₂SO₄), filtered and concentrated. The residue was purified on silca gel with iso-hexane containing EtOAc (10-30%) to give the title compound.

LC/MS t=4.07 min, [MH⁺] 507, 509.

### Example 371

### 3-{2-[5-Chloro-2-(benzyloxy)-phenyl]-5-methyl-pyrrol-1-yl}-N-(pyridin-2-ylmethyl)-benzamide

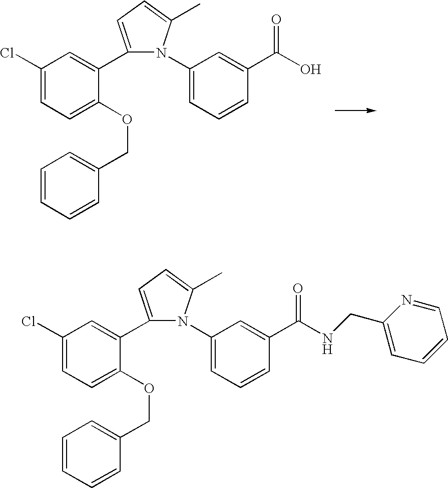

3-{2-[5-Chloro-2-(benzyloxy)-phenyl]-5-methyl-pyrrol-1-yl}-benzoic acid (20 mg) was dissolved in DCM (0.5 mL). EDC (12 mg) and HOBt (9 mg) were added and the mixture stirred for 5 minutes before 2-(aminomethyl)pyridine (11 μL) was added. The mixture was stirred overnight then diluted with EtOAc and washed sequentially with saturated NH₄Cl and saturated NaHCO₃, dried (Na₂SO₄), filtered and concentrated. The residue was purified on silica gel with EtOAc to give the title compound.

LC/MS t=3.77 min, [MH⁺] 508, 510; [MH⁻] 506, 508.

### Example 372

### 3-{2-[5-Chloro-2-benzyloxy)-phenyl]-5-methyl-pyrrol-1-yl}-N-(pyridin-3-ylmethyl)-benzamide

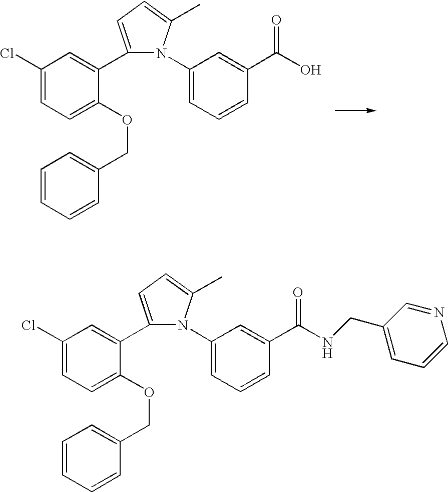

3-{2-[5-Chloro-2-(benzyloxy)-phenyl]-5-methyl-pyrrol-1-yl}-benzoic acid (20 mg) was dissolved in DCM (0.5 mL). EDC (12 mg) and HOBt (9 mg) were added and the mixture stirred for 5 minutes before 3-(aminomethyl)pyridine (11 μL) was added. The mixture was stirred overnight then diluted with EtOAc and washed sequentially with saturated NH₄Cl and saturated NaHCO₃, dried (Na₂SO₄), filtered and concentrated. The residue was purified on silica gel with EtOAc to give the title compound.

LC/MS t=3.63 min, [MH⁺] 508, 510; [MH⁻] 506, 508.

### Example 373

### 3-{2-[5-Chloro-2-(benzyloxy)-phenyl]-5-methyl-pyrrol-1-yl}-N-(pyridin-4-ylmethyl)-benzamide

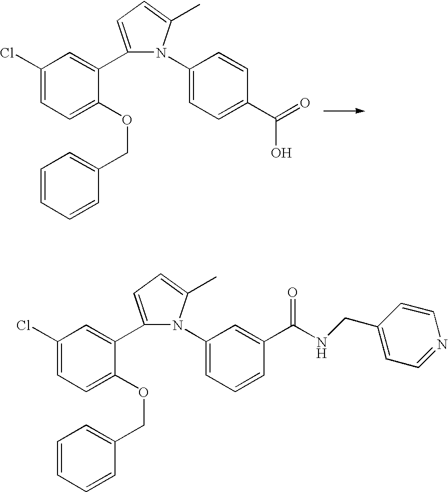

3-{2-[5-Chloro-2-(benzyloxy)-phenyl]-5-methyl-pyrrol-1-yl}-benzoic acid (20 mg) was dissolved in DCM (0.5 mL). EDC (1 2 mg) and HOBt (9 mg) were added and the mixture stirred for 5 minutes before 4-(aminomethyl)pyridine (11 μL) was added. The mixture was stirred overnight then diluted with EtOAc and washed sequentially with saturated NH₄Cl and saturated NaHCO₃, dried (Na₂SO₄), filtered and concentrated. The residue was purified on silica gel with EtOAc to give the title compound.

LC/MS t=3.63 min, [MH⁺] 508, 510; [MH⁻] 506, 508.

### Example 374

### 3-{2-[5-Chloro-2-(benzyloxy)-phenyl]-5-methyl-pyrrol-1-yl}-N-(benzyl)-benzamide

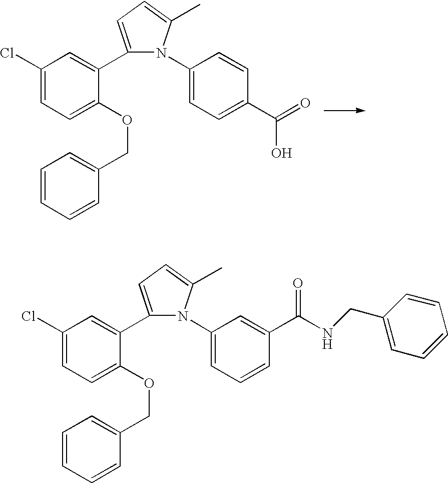

3-{2-[5-Chloro-2-(benzyloxy)-phenyl]-5-methyl-pyrrol-1-yl}-benzoic acid (20 mg) was dissolved in DCM (0.5 mL). EDC (1 2 mg) and HOBt (9 mg) were added and the mixture stirred for 5 minutes before benzylamine (11 μL) was added. The mixture was stirred overnight then diluted with EtOAc and washed sequentially with saturated NH₄Cl and saturated NaHCO₃, dried (Na₂SO₄), filtered and concentrated. The residue was purified on silica gel with EtOAc to give the title compound.

LC/MS t=4.06 min, [MH⁺] 507, 509; [MH⁻] 505, 507.

### Example 375

### 4-{2-[5-Bromo-2-(benzyloxy)-phenyl]-5-methyl-pyrrol-1-yl}-methylsulfonyl benzene

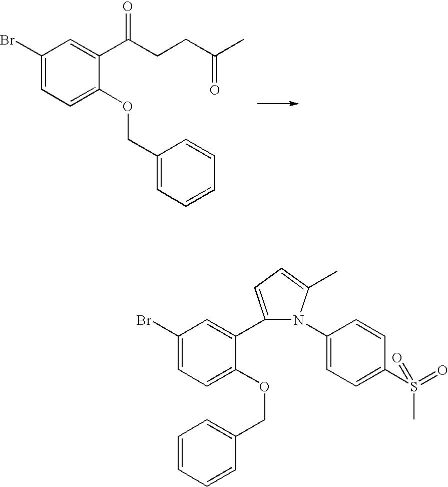

1-[5-Bromo-2-benzyloxy)-phenyl]-pentane-1,4-dione (262 mg), 4-(methylsulfonyl)aniline hydrochloride (198 mg) and TEA (0.10 mL) were heated at reflux in toluene (7.8 mL) for 24 hours. The mixture was diluted with EtOAc and washed sequentially with 2M HCl and saturated NaHCO₃, dried (Na₂SO₄), filtered and concentrated. The residue was purified on silica gel with iso-hexane containing EtOAc (10-30%) to give the title compound.

LC/MS t=3.86 min, [MH⁺] 496, 498.

### Example 376

### 3-{2-[5-Bromo-2-2,4-difluoro-benzyloxy)-phenyl]-5-methyl-pyrrol-1-yl}-6-methyl-benzoic acid

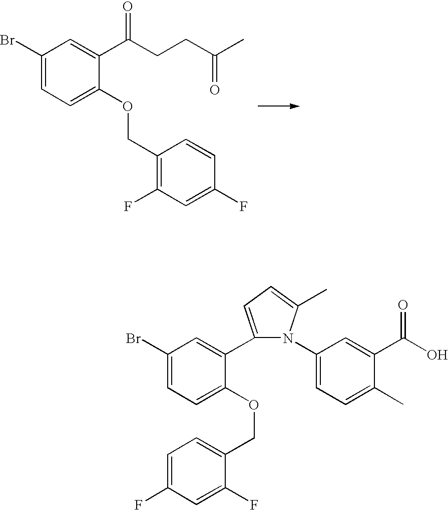

1-[5-Bromo-2-(2,4-difluoro-benzyloxy)-phenyl]-pentane-1,4dione (1 14 mg), 5-amino-2-methyl-benzoic acid (52 mg) and pTSA (cat) were heated in NMP (1.5 mL) at 150° C. for 10 minutes. The mixture was diluted with Et₂O washed with 2M HCl, dried (Na₂SO₄), filtered and evaporated. The residue was purified by MDAP, to give the title compound (79 mg).

¹H NMR (400 MHz, CDCl₃) 2.15 (3H, s), 2.62 (3H, s), 4.75 (2H, s), 6.12 (1H, d, J=3 Hz), 6.28 (1H, J=3 Hz), 6.56 (1H, d, J=9 Hz), 6.72-6.82 (2H, m), 6.92-6.99 (1H, m), 7.01 (1H, dd, J=2 Hz, J=8 Hz), 7.12 (1H, d, J=8 Hz), 7.22 (1H, dd, J=2 Hz, J=9 Hz), 7.38 (1H, d, J=3 Hz), 7.74 (1H, d, J=2 Hz).

LC/MS t=4.11 min, [MH⁺] 512, 514; [MH⁻] 510, 512

### Example 377

### 4-{2-[5-Chloro-2-(benzyloxy)-phenyl]-5-ethyl-pyrrol-1-yl}-benzene sulfonamide

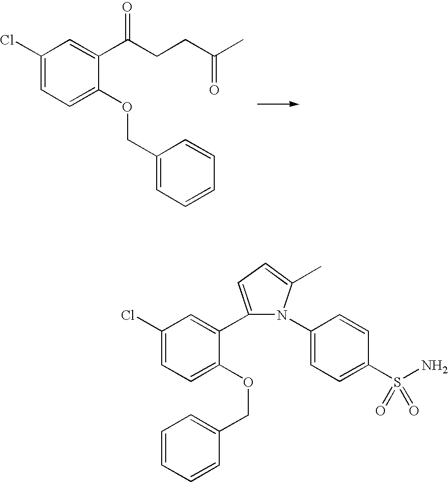

1-[5-Chloro-2-benzyloxy)-phenyl]-pentane-1,4-dione (508 mg, 1.61 mmol), sulfanilamide (336 mg, 1.95 mmol) and pTSA (cat) were heated at relux in toluene (8 mL) for 3.5 days. The mixture was diluted with EtOAc and washed sequentially with 2M HCl and saturated NaHCO₃, dried (Na₂SO₄), filtered and concentrated. The residue was purified on silica gel with iso-hexane containing EtOAc (20-40%) to give the title compound.

LC/MS t=3.68 min, [MH⁺] 453, 455; [MH⁻] 451, 453.

### Example 378

### 3-{2-[6-Chloro-2-(benzyloxy)-phenyl]-5-methyl-pyrrol-1-yl}-benzamide

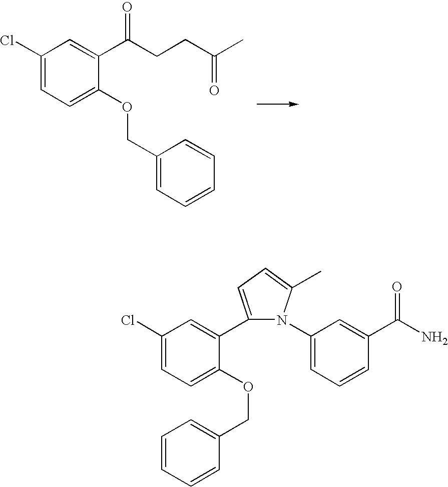

1-[5-Chloro-2-benzyloxy)-phenyl]-pentane-1,4-dione (303 mg, 0.96 mmol), 3-aminobenzamide (212 mg, 1.56 mmol) and pTSA (cat) were heated at relux in toluene (5 mL) for 2.5 hours. The mixture was diluted with EtOAc and washed sequentially with 2M HCl and saturated NaHCO₃, dried (Na₂SO₄), filtered and concentrated. The residue was purified on silica gel with iso-hexane containing EtOAc (30-50%) to give the title compound (293 mg, 74%).

LC/MS t=3.61 min, [MH⁺] 417, 419.

### Example 379

### 3-{2-[5-Chloro-2-(benzyloxy)-phenyl]-5-methyl-pyrrol-1-yl}-benzonitrile

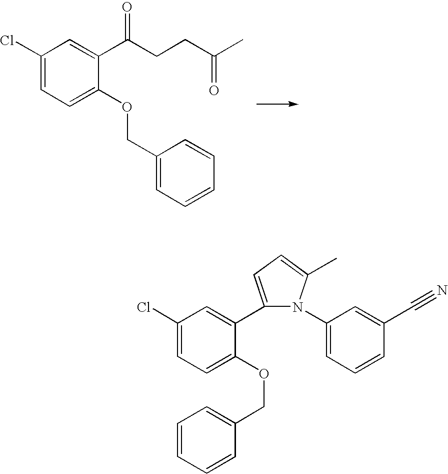

1-[5-Chloro-2-benzyloxy)-phenyl]-pentane-1,4-dione (254 mg, 0.80 mmol), 3-aminobenzonitrile (212 mg, 1.1 3 mmol) and pTSA (cat) were heated at relux in toluene (4 mL) for 2.5 hours. The mixture was diluted with EtOAc and washed sequentially with 2M HCl and saturated NaHCO₃, dried (Na₂SO₄), filtered and concentrated. The residue was purified on silica gel with iso-hexane containing EtOAc (2-5%) to give the title compound (259 mg, 81%).

LC/MS t=3.98 min, [MH⁺] 399, 401.

### Example 380

### 3-{2-[5-Trifluoromethyl-2-(benzyloxy)-phenyl]-5-methyl-pyrrol-1-yl}-6-fluoro-benzoic acid

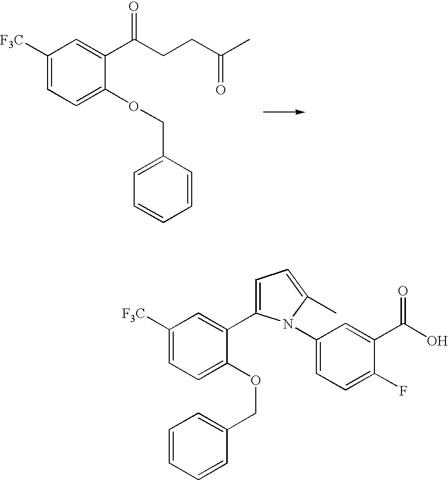

Procedure as for 3-{2-[5-trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.14 (3H, s), 4.87 (2H, s), 6.13 (1H, d, J=3 Hz), 6.34 (1H, d, J=3 Hz), 6.76 (1H, d, J=8 Hz), 6.98 (1H, t J=9 Hz), 7.07-7.14 (3H, m), 7.22-7.41 (4H, m's excess), 7.50 (1H, s), 7.69 (1H, dd, J=6 Hz, 3 Hz).

LC/MS t=4.01 min [MH⁺] 470.

### Example 381

### 3-{2-[5-Trifluoromethyl-2-(benzyloxy)-phenyl]-5-methyl-pyrrol-1-yl}-5-acetylamino-benzoic acid

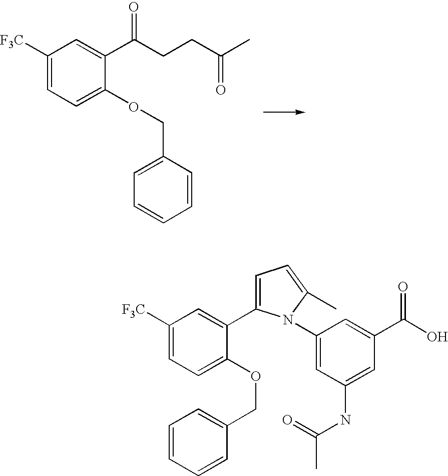

Procedure as for 3-{2-[5trifluoromethyl-2-(4-fluoro-benzyloxy)-phenyl]-5-methyl-pyrrol-1-yl}-6-chloro-benzoic acid using the appropriate amine, to give the title compound.

¹H NMR (400 MHz, CDCl₃) 2.11 (3H, s), 2.16 (3H, s), 4.87 (2H, s), 5.22 (1H, s), 6.13 (1H, d, J=3 Hz), 6.35 (1H, d, J=3 Hz), 6.73 (1H, d, J=8 Hz), 7.06-7.13 (3H, m), 7.24-7.50 (7H, m), 8.02 (1H, brs).

LC/MS t=3.72 min [MH⁺] 509.

### Example 382

### 3-{2-[5-Fluoro-2-(4-fluoro-benzyloxy)-phenyl]-5-methyl-pyrrol-1-yl}-5-amino-benzoic acid

### a) 3-{2-[5-fluoro-2-(4-Fluoro-benzyloxy)-phenyl]-5-methyl-pyrrol-1-yl}-5-amino-benzoic acid methyl ester

Procedure as for 3-{2-[5-fluoro-2-(4-fluoro-benzyloxy)-phenyl]-5-methyl-pyrrol-1-yl}-6-difluoromethoxy-benzoic acid methyl ester.

LC/MS t=3.77 min [MH⁺] 449.

### b) 3-{2-[5-fluoro-2-(4-Fluoro-benzyloxy)-phenyl]-5-methyl-pyrrol-1-yl}-5-amino-benzoic acid

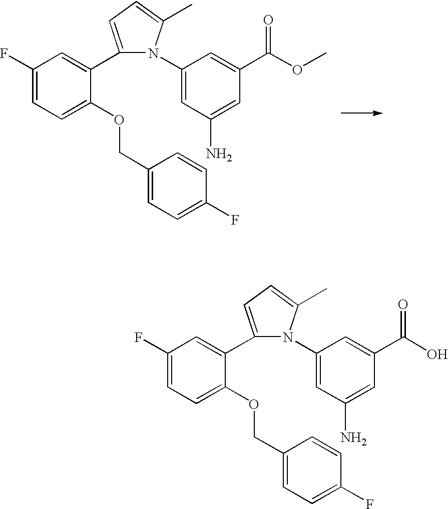

Procedure as for 3-{2-[5-fluoro-2-(4-fluoro-benzyloxy)-phenyl]-5-methyl-pyrrol-1-yl}-6-difluoromethoxy-benzoic acid using the appropriate amine.

¹H-NMR (400 MHz, d6-DMSO) 2.09 (3H, s), 4.91 (2H, s), 6.04 (1H, d, J=3 Hz), 6.27 (1H, d, J=3 Hz), 6.60 (1H, s), 6.79 (1H, dd, J=3 Hz, 9 Hz), 6.85 (1H, s), 6.88-7.00 (2H, m), 7.15-7.24 (3H, m), 7.26-7.33 (2H, m).

LC/MS t=3.58 min [MH⁺] 435

### Example 383

### 3-{2-[5-Bromo-2-(2,4-difluoro-benzyloxy)-phenyl)]-5-methypyrrol-1-yl}-6-difluoromethoxy-benzoic acid

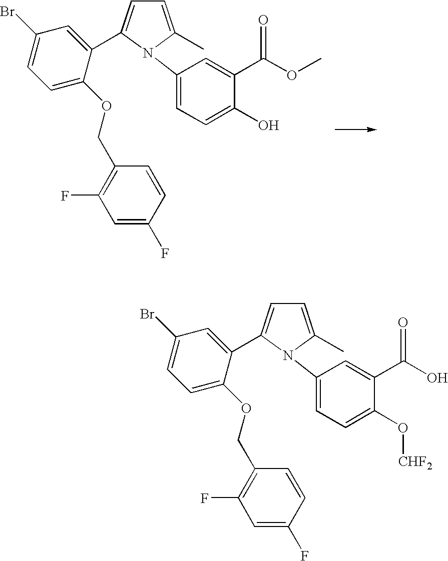

3-{2-[5-Bromo-2-(2,4-difluoro-benzyloxy)-phenyl]-5-methylpyrrol-1-yl}-6-hydroxy-benzoic acid methyl ester (217 mg, 0.4 mmol) was treated with anhydrous potassium carbonate (100 mg, 0.6 mmol) and sodium chlorodifluoroacetate (100 mg, 7 mmol) in dimethylformamide (4.5 ml) at room temperature. The reaction mixture was then gradually heated to 100° C. and kept at 100° C. for 3 hours. The reaction mixture was cooled, filtered through celite and thoroughly washed with DCM. The filtrate was washed with brine, dried (MgSO4), filtered and evaporated. The residue was purified on a Water's sep-pack (10 mg) with Et₂O/iso-hexane, to give the intermediate ester, which was hydrolysed in the same way as 3-{2-[5-bromo-2-(2,4-difluoro-benzyloxy)-phenyl)]-5-methypyrrol-1-yl}-6-difluoromethoxy-benzoic acid. (65 mg, 27%).

¹H NMR (400 MHz, CDCl₃) 2.12 (3H, s), 4.73 (2H, s), 6.12 (1H, d, J=3 Hz), 6.28 (1H, d, J=3 Hz), 6.39-6.87 (4H, m), 6.99-7.15 (3H, m), 7.23-7.29 (1H, m excess), 7.38 (1H, d, J=2 Hz), 7.67 (1H, d, J=2 Hz).

LC/MS t=4.08 min [MH⁺] 566

### Example 384

### 2-(3-{2-[5-Bromo-2-(2,4-difluoro-benzyloxy)-phenyl]-5-methyl-pyrrol-1-yl}-phenyl)-1H-benzoimidazole

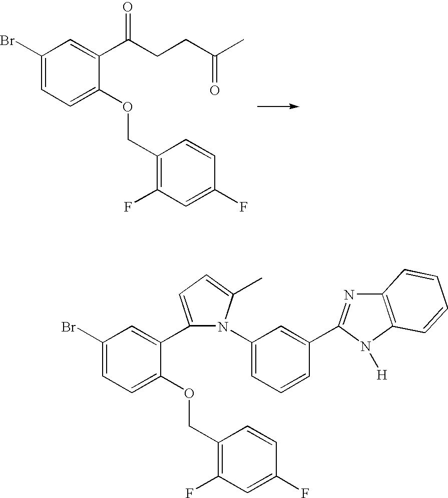

1-[5-Bromo-2-(2,4-difluoro-benzyloxy)-phenyl]-pentane-1,4-dione (100 mg, 0.25 mmol), 3-(1H-benzoimidazol-2-yl)-phenylamine (63 mg, 0.3 mmol) {Brana et al, J. Het. Chem., 1990, 27(5), 1177-80), and p-toluenesulfonic acid (1 0 mg) were heated in toluene (1 ml) at reflux for 19 hours. Upon cooling, the mixture was diluted with EtOAc (3 ml) and washed with 2M HCl (2 ml), saturated NaHCO₃, dried (Na₂SO₄), filtered and concentrated. The residue was purified by MDAP, to give the title compound (77 mg, 53%).

LC/MS t=4.04 min [MH⁺] 570/572.

### Example 385

### 5-(3-{2-[5-Bromo-2-(2,4-difluoro-benzyloxy)-phenyl]-5-methyl-pyrrol-1-(1-yl}-phenyl)-1H-tetrazole

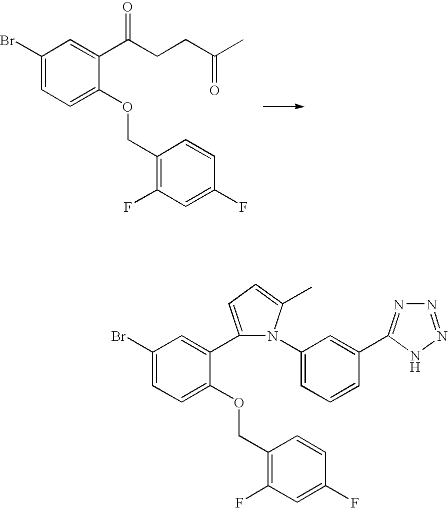

Preparation as for 2-(3-{2-[5-bromo-2-(2,4-difluoro-benzyloxy)-phenyl]-5-methyl-pyrrol-1-yl}-phenyl)-1H-benzoimidazole using the appropriate amine to give the title compound (80 mg, 60%).

¹H NMR (400 MHz, CDCl₃) 2.20 (3H, s), 4.74 (2H, s), 6.14 (1H, d, J=2 Hz), 6.31 (1H, d, J=2 Hz), 6.57 (1H, d, J=10 Hz), 6.72 (2H, m), 7.01 (1H, t, J=8 Hz), 7.11 (1H, bd, J=8 Hz), 7.22 (1H, dd, J=8 Hz), 7.40 (2H, m), 7.73 (1H, bs), 7.99 (1H, bd, J=8 Hz).

LC/MS t=4.38 min [MH⁺] 522/524.

### Example 386

### 2-(3-{2-[5-Bromo-2-(2,4difluoro-benzyloxy)-phenyl]-5-methyl-pyrrol-1-yl}-phenyl)-5-methyl-[1,3,4]oxadiazole

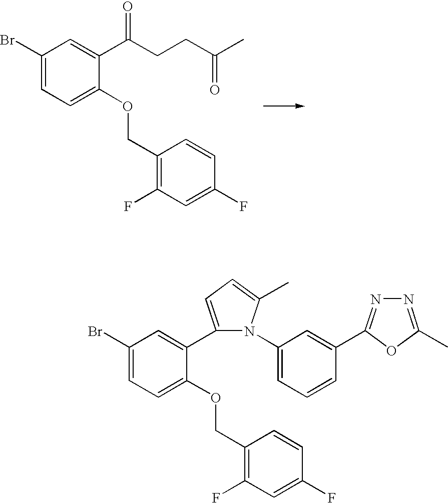

Preparation as for 2-(3-{2-[5-bromo-2-(2,4-difluoro-benzyloxy)-phenyl]-5-methyl-pyrrol-1-yl}-phenyl)-1H-benzoimidazole using the appropriate amine (Hoefle et al., U.S. Pat. No. 4,824,843) (31 mg, 22%).

LC/MS t=3.99 min [MH⁺] 536/538.

### Example 387

### 2-(4-{2-[5-Bromo-2-(2,4difluoro-benzyloxy)-phenyl]-5-methyl-pyrrol-1yl}-phenyl)-1,1,1,3,3,3-hexafluoro-propan-2-ol

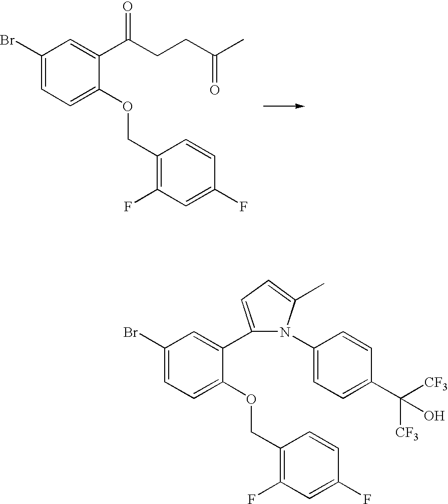

Preparation as for 2-(3-{2-[5-bromo-2-(2,4-difluoro-benzyloxy)-phenyl]-5-methyl-pyrrol-1-yl}-phenyl)-1H-benzoimidazole using the appropriate amine (5 mg, 3%).

¹H NMR (400 MHz, CDCl₃) 2.20 (3H, s), 4.61 (2H, s), 6.13 (1H, d, J=3 Hz), 6.33 (1H, d, J=4 Hz), 6.55 (1H, d, J=8 Hz), 6.76-6.86 (2H, m), 7.03-7.06 (2H, m), 7.08-7.16 (1H, m), 7.22 (1H, dd, J=2, 8 Hz), 7.30 (1H, d, J=2 Hz).7.59 (2H, d, J=8 Hz).

LC/MS t=4.26 min [MH⁺] 620/622.

### Example 388

### 5-(4-{2-[5-Bromo-2-(2,4,6-trifluoro-benzyloxy)-phenyl]-5-methyl-pyrrol-1-yl}-benzyl)-1H-tetrazole

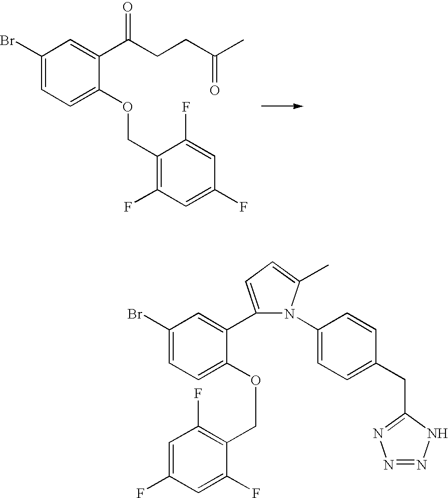

Procedure as for 3-{2-[5-bromo-2-(benzyloxy)-phenyl]-5-methyl-pyrrol-1-yl}-6-acetylamino-benzoic acid methyl ester using the appropriate amine (Kees et al, J. Med. Chem, 1992, 35(5), 944-53), however further purification was achieved using the MDAP.

¹H-NMR (400 MHz, CDCl₃) 2.13 (3H, s), 4.34 (2H, s), 4.78 (2H, s), 6.06 (1H, d, J=3 Hz), 6.29 (1H, d, J=3 Hz), 6.67 (2H, t, J=8 Hz), 6.73 (1H, d, J=9 Hz), 6.99 (2H, d, J=9 Hz), 7.13 (1H, d, J=2 Hz), 7.17 (2H, d, J=9 Hz), 7.22 (1H, dd, J=3 Hz, 9 Hz).

LC/MS t=3.68 min [MH⁺] 554/556.

### Example 389

### 5-(4-{2-[5-Bromo-2-(2,4-difluoro-benzyloxy)-phenyl]-5-methyl-pyrrol-1-yl}-phenyl)-1H-imidazole

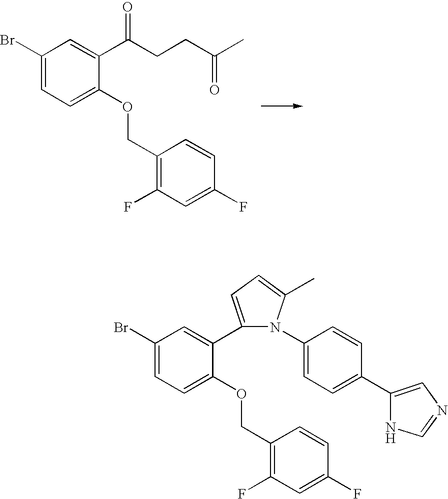

Procedure as for 4-{2-[5-bromo-2-(2,4-difluoro-benzyloxy)-phenyl]-5-methyl-pyrrol-1-yl}-benzamide using the appropriate amine to give title compound.

LC/MS t=3.38 min [MH⁺] 519/521.

### Example 390

### 1-(4-{2-[5-Bromo-2-(2,4-difluoro-benzyloxy)-phenyl]-5-methyl-pyrrol-1-yl}-phenyl)-ethanone

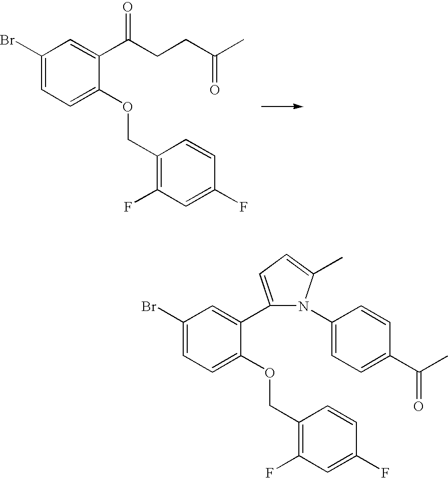

1-[5-Bromo-2-(2,4-difluoro-benzyloxy)-phenyl]-pentane-1,4-dione (0.10g, 0.25 mmol), 4-ethynyl-phenylamine (0.035g, 0.3 mmol) and p-TSA (0.009g, 0.05 mmol) were refluxed in toluene (1.5 ml) for 18 hours under a nitrogen atmosphere. The solvent was removed in vacuo and the resultant residue purified by MDA to give the title compound.

¹H-NMR (400 MHz, CDCl₃) 2.17 (3H, s), 2.58 (3H, s), 4.70 (2H, s), 6.13 (1H, d, J=3 Hz), 6.30 (1H, d, J=3 Hz), 6.56 (1H, d, J=9 Hz), 6.75-6.83 (2H, m), 6.94 (1H, q, J=7 Hz), 7.00-7.04 (2H, m), 7.23 (1H, dd, J=3 Hz, 9 Hz), 7.37 (1H, d, J=3 Hz), 7.79-7.84 (2H, m).

LC/MS t=4.08 min [MH⁺] 498/500.

### Example 391

### 4-{2-[2-(Benzyloxy)-phenyl]-5-methyl-pyrrol-1-yl}-benzoic acid

### a) 4-{2-[2-(Benzyloxy)-phenyl]-5-methyl-pyrrol-1-yl}-benzoic acid methyl ester

1-[2-(benzyloxy)-phenyl]-pentane-1,4-dione (540 mg), 4-amino-benzoic acid methyl ester (355 mg) and pTSA (cat) were heated at reflux in toluene (20 mL) for 5 hours. The mixture was diluted with EtOAc and washed sequentially with 2M HCl and saturated NaHCO₃, dried (Na₂SO₄), filtered and concentrated. The residue was purified on silica gel with iso-hexane containing EtOAc (3-5%) to give the title compound (684 mg)

Rf 0.61 (30% EtOAc in hexanes).

### b) 4-{2-[2-(Benzyloxy)-phenyl]-5-methyl-pyrrol-1-yl}-benzoic acid

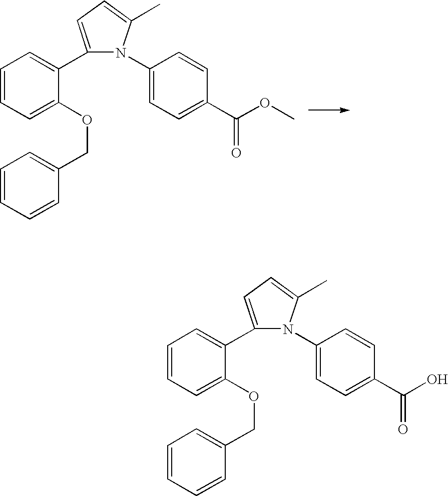

3-{2-[2-(benzyloxy)-phenyl]-5-methyl-pyrrol-1-yl}-benzoic acid methyl ester (214 mg) was heated in a reacti-vial at 85° C. in a mixture of DMF (4.0 mL) and 2M NaOH (2.0 mL) for 24 hours. The mixture was diluted with EtOAc and washed with 2M HCl, dried (Na₂SO₄), filtered and evaporated. The residue was chromatographed on silica gel, with iso-hexane-EtOAc-AcOH (90:10:2 to 70:30:2) as eluant, to give the title compound (140 mg).

Rf 0.35 iso-hexane-EtOAc-AcOH (70:30:2)

LC/MS t=3.57 min [MH⁺] 384, [MH⁻] 382.

### Example 392

### 4-{2-[5-Chloro-2-(benzyloxy)-phenyl]-5-methyl-pyrrol-1-yl}-benzoic acid

### a) 4-{2-[5-Chloro-2-(benzyloxy)-phenyl]-5-methyl-pyrrol-1-yl}-benzoic acid methyl ester

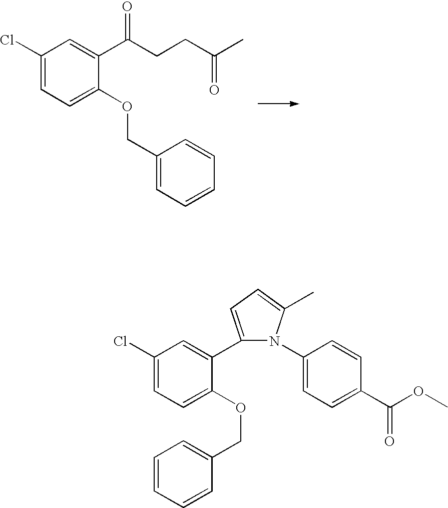

1-[5-Chloro-2-(benzyloxy)-phenyl]-pentane-1,4-dione (1.0139), 4-amino-benzoic acid methyl ester (616 mg) and pTSA (cat) were heated at reflux in toluene (34 mL) overnight. The mixture was diluted with EtOAc and washed sequentially with 2M HCl and saturated NaHCO₃, dried (Na₂SO₄), filtered and concentrated. The residue was purified on silica gel with iso-hexane containing EtOAc to give the title compound (815 mg)

Rf 0.74 (30% EtOAc in iso-hexane)

### b) 4-{2-[5-Chloro-2-(benzyloxy)-phenyl]-5-methyl-pyrrol-1-yl}-benzoic acid

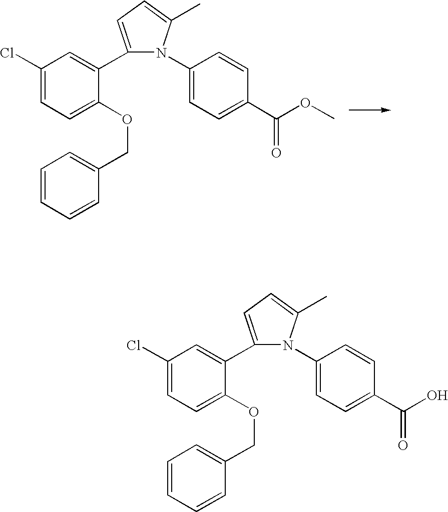

4-{2-[5-Chloro-2-(benzyloxy)-phenyl]-5-methyl-pyrrol-1-yl}-benzoic acid methyl ester (152 mg) was heated at reflux in a mixture of EtOH (3.0 mL) and 2M NaOH (1.0 mL) overnight. The mixture was diluted with EtOAc and washed with 2M HCl, dried (Na₂SO₄), filtered and evaporated to give the title compound.

LC/MS t=3.73 min, [MH⁺] 418; [MH⁻] 416, 418.

### Example 393

### 4-{2-[5-Bromo-2-(benzyloxy)-phenyl]-5-methyl-pyrrol-1-yl}-benzoic acid

### a) 4-{2-[5-Bromo-2-(benzyloxy)-phenyl]-5-methyl-pyrrol-1-yl}-benzoic acid methyl ester

1-[5-Bromo-2-(benzyloxy)-phenyl]-pentane-1,4-dione (1.020g), 4-amino-benzoic acid methyl ester (581 mg) and pTSA (cat) were heated at reflux in toluene (30 mL) overnight.

The mixture was diluted with EtOAc and washed sequentially with 2M HCl and saturated NaHCO₃, dried (Na₂SO₄), filtered and concentrated. The residue was purified on silica gel, with iso-hexane containing EtOAc (5%) as eluant, to give the title compound (757 mg)

¹H NMR (400 MHz, CDCl₃) 2.15 (3H, s), 3.91 (3H, s), 4.68 (2H, s), 6.16 (1H, d, J=3 Hz), 6.31 (1H, d, J=3 Hz), 6.50 (1H, d, J=9 Hz), 6.97-7.06 (4H, m), 7.19 (1H, dd, J=3 Hz, J=9 Hz), 7.23-7.34 (3H, m's excess), 7.39 (1H, d, J=2 Hz), 7.88 (2H, d, J=8 Hz).

### b) 4-{2-[5-Bromo-2-(benzyloxy)-phenyl]-5-methyl-pyrrol-1-yl}-benzoic acid

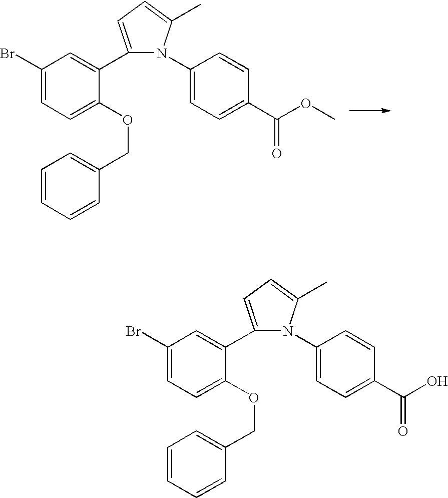

4-{2-[5-Bromo-2-(benzyloxy)-phenyl]-5-methyl-pyrrol-1-yl}-benzoic acid methyl ester (1 52 mg) was heated at reflux in a mixture of EtOH (3.0 mL) and 2M NaOH (1.5 mL) for 3 hours. The mixture was diluted with EtOAc and washed with 2M HCl, dried (Na₂SO₄), filtered and evaporated to give the title compound.

LC/MS t=4.01 min, [MH⁺] 462; [MH⁻] 460.

### Example 394

### 3-{2-[5-Chloro-2-(2,4-difluoro-benzyloxy)-phenyl]-5-methyl-pyrrol-1-yl}-N-1-(3,5-dimethyl-isoxazol-4-yl)-methanoyl-benzenesulfonamide

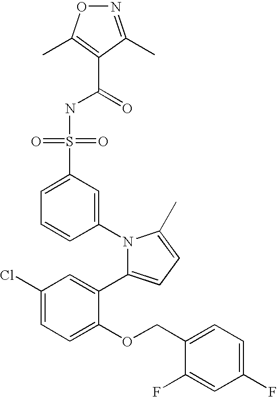

Prepared in the same way as 3-{2-[5-chloro-2-(2,4-difluoro-benzyloxy)-phenyl]-5-methyl-pyrrol-1-yl}-N-[1-phenyl-methanoyl]-benzenesulfonamide.

LC/MS t=4.51 min, [MH⁺] 612, 614; [MH⁻] 610, 612.

Microwave

Emrys Optimiser and Smith Creator (300 Watt) supplied by Personal Chemistry.

Mass Directed Auto-Purification System

Hardware

Waters 600 gradient pump

Waters 2700 sample manager

Waters Reagent manager

Waters 996 photodiode array detector

Micromass ZQ mass spectrometer

Gilson 202 fraction collector

Gilson Aspec waste collector

Software

Micromass Masslynx version 3.5

Column

The column used is a Supelcosil™0 ABZ+PLUS, the dimensions of which are 21.2 mm×100 mm. The stationary phase particle size is 5 μm.

Solvents

A: Aqueous solvent=Water+0.1% Formic Acid

B: Organic solvent=Acetonitrile+0.1% Formic Acid

Make up solvent=Methanol:Water 80:20+2 mMol Ammonium Acetate

Needle rinse solvent=Methanol:Water:Dimethylsulfoxide 80:10:10

Methods

There are five methods used depending on the analytical retention time of the compound of interest. They all have a 15-minute runtime, which comprises of a 10-minute gradient followed by a 5 minute column flush and re-equilibration step.

MDP 1.5-2.2=0-20% B

MDP 2.0-2.8=0-30% B

MDP 2.5-3.0=15-55% B

MDP 2.8-4.0=30-85% B

MDP 3.8-5.5=50-99% B

Flow Rate

All of the above methods have a flow rate of 20 ml/mins

LCMS Systems

Hardware

Agilent 1100 gradient pump

Agilent 1100 Autosampler

Agilent 1100 PDA Detector

Agilent 1100 Degasser

Micromass ZQ mass spectrometer

PL-ELS 1000

Software

Micromass Masslynx versions 3.5/4.0

Column

The column used is a Supelcosil™ ABZ+PLUS, the dimensions of which are 4.6 mm×33 mm. The stationary phase particle size is 3 μm.

Solvents

A: Aqueous solvent=10 mMol Ammonium Acetate+0.1% Formic Acid

B: Organic solvent=95% Acetonitrile+0.05% Formic Acid

Method

The generic method used has 5.5 minute runtime, which comprises of a 4,7-minute gradient (0-100% B) followed by a 0.6 minute column flush and 0.2 minute re-equilibration step.

Flow Rate

The above method has a flow rate of 3 ml/mins

NMR

Hardware

Bruker 400 MHz Ultrashield™

Bruker B-ACS60 Autosampler

Bruker Advance 400 Console

Software

User interface—NMR Kiosk

Controlling software—XWin NMR version 3.0

Assays for Determining Biological Activity

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 □g/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

By application of this technique, compounds of the examples had an antagonist pIC₅₀ value of between 7.0 and 9.5 at EP₁ receptors and pIC50 value of <6.0 at EP₃ receptors. Preferred compounds have an antagonist pIC₅₀ value of greater than 8.0 at EP₁ receptors.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1. A compound of formula (I): 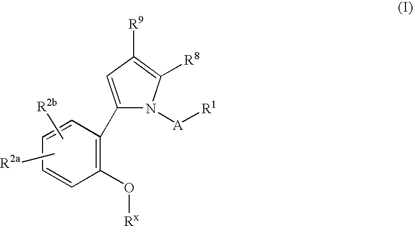
wherein:
A is an optionally substituted aryl group, or an optionally substituted 5- or 6-membered heterocyclyl ring, or an optionally substituted bicyclic heterocyclyl group;
R¹ is CO₂H, CN, CONR⁵R⁶, CH₂CO₂H, optionally substituted alkyl, optionally substituted alkenyl, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} independently are hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} is optionally substituted alkyl wherein 1 or 2 of the non-terminal carbon atoms may optionally be replaced by a group independently selected from NR⁴, O and SOₙ, wherein n is 0, 1 or 2: or R^{x} may be optionally substituted CQ₂-heterocyclyl, optionally substituted CQ₂-bicyclic heterocyclyl or optionally substituted CQ₂-aryl;
R⁴ is hydrogen or an optionally substituted alkyl;
R⁵ is hydrogen or an optionally substituted alkyl;
R⁶ is hydrogen or optionally substituted alkyl, optionally substituted heteroaryl, optionally substituted SO₂aryl, optionally substituted SO₂alkyl, optionally substituted SO₂heteroaryl, CN, optionally substituted CQ₂aryl, optionally substituted CQ₂heteroaryl or COR⁷;
R⁷ is hydrogen, optionally substituted alkyl, optionally substituted heteroaryl or optionally substituted aryl;
R⁸ is hydrogen, CF₃, or alkyl;
R⁹ is hydrogen, CF₃ or alkyl;
Q is independently selected from hydrogen and CH₃;
wherein when A is a 6-membered ring the R¹ substituent and pyrrole ring are attached to carbon atoms 1,2-, 1,3- or 1,4- relative to each other, and when A is a five-membered ring or bicyclic heterocyclyl group the R¹ substituent and pyrrole ring are attached to substitutable carbon atoms 1,2- or 1,3- relative to each other;
or a pharmaceutically acceptable derivative thereof.

2. A compound according to claim 1 wherein A is selected from phenyl, naphthyl, indolyl, pyridyl, pyridazinyl, pyrazinyl or pyrimidinyl, all of which may be optionally substituted.

3. A compound according to claim 1 wherein R¹ is CO₂H, CN, CONR⁴R⁵, optionally substituted CONR⁵SO₂aryl, optionally substituted CONR⁵SO₂heteroaryl, optionally substituted CONR⁵aryl, optionally substituted CONR⁵heteroaryl, CONR⁵SO₂C₁₋₆alkyl, optionally substituted CONR⁵SO₂heteroaryl, optionally substituted CONR⁵CQ₂aryl, optionally substituted CONR⁵CQ₂heteroaryl, optionally substituted C₁₋₆alkyl, SO₂C₁₋₆alkyl, SO₂NR⁴R⁵, optionally substituted SO₂NR⁵COaryl, optionally substituted SO₂NR⁵COheteroaryl, SO₂NR⁵COC₁₋₆alkyl, optionally substituted SO₂NR⁵CQ₂aryl, optionally substituted SO₂NR⁵CQ₂heteroaryl; COC₁₋₆alkyl, 2H-tetrazol-5-yl-methyl, optionally substituted bicyclic heterocycyl, or optionally substituted heterocyclyl; wherein R⁴ and R⁵ are each selected from hydrogen and C₁₋₄alkyl, and Q is selected from hydrogen and CH₃.

4. A compound according to claim 1 wherein A is a six membered ring and R¹ substituent is attached to the group A in the 3- or 4-position relative to the bond attaching A to the pyrrole ring.

5. A compound according to claim 1 which is a compound of formula (Ia): 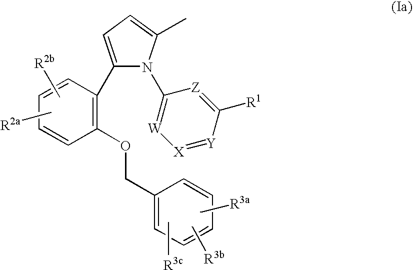
wherein:
R¹ is CO₂H;
R^{2a} and R^{2b} are independently selected from hydrogen, halo, phenyl, optionally substituted C₁₋₆alkyl e.g. C₁₋₄alkyl and CF₃, CN, SC₁₋₆alkyl, or SO₂C₁₋₆alkyl;
R^{3a}, R^{3b}, and R^{3c} are independently selected from hydrogen, halo, optionally substituted OC₁₋₆alkyl, phenyl or optionally substituted C₁₋₆alkyl;
W, X, Y and Z are each CR¹² or N wherein at least two of W, X, Y or Z is CR¹²; and when each of W, X, Y, and Z is CR¹² then each R¹² is independently selected from hydrogen, halogen, C₁₋₄haloalkyl, C₁₋₄haloalkoxy, NR⁴R⁵, NR⁵COC₁₋₆alkyl, NR⁵SO₂C₁₋₆alkyl, OR⁵, C₁₋₆alkyl, SO₂C₁₋₆alkyl, NR⁵COCH₂OC₁₋₆alkyl, NR⁵COCH₂heterocyclyl wherein R⁴ and R⁵ are each independently selected from hydrogen and C₁₋₄alkyl; and NR¹⁰R¹¹ wherein R¹⁰ and R¹¹ together with the nitrogen atom to which they are attached form an optionally substituted 5- or 6-membered aliphatic heterocyclic ring wherein one of the ring carbons may be optionally replaced by another heteroatom selected from O and SOₙ wherein n is 0, 1 or 2., and when at least one of W, X, Y and Z represents N then each R¹² is selected from hydrogen and NH₂;
or a pharmaceutically acceptable derivative thereof.

6. A compound selected from
3-{2-[5-Bromo-2-(4-fluoro-benzyloxy)-phenyl]-5-methyl-pyrrol-1-yl}-benzoic acid
5-{2-[5-Chloro-2-(4-chloro-benzyloxy)-phenyl]-5-methyl-pyrrol-1-yl}-nicotinic acid
3-{2-[5-Chloro-2-(4-fluorobenzyloxy)-phenyl]-5-methylpyrrol-1-yl}-5-acetylamino-benzoic acid
3-{2-[5-Chloro-2-(4-fluorobenzyloxy)-phenyl]-5-methylpyrrol-1-yl}-5-methyl-benzoic acid
3-{2-[5-Chloro-2-(2,4-difluorobenzyloxy)-phenyl]-5-methylpyrrol-1-yl}-6-methyl-benzoic acid
3-{2-[5-Chloro-2-(2,4-difluorobenzyloxy)phenyl]-5-methyl-pyrrol-1-yl}-6-chloro-benzoic acid
3-{2-[5-Bromo-2-(2,4-difluorobenzyloxy)-phenyl]-5-methylpyrrol-1-yl}-5-acetylamino-benzoic acid
3-{2-[5-Methanesulfonyl-2-(2,4-difluoro-benzyloxy)-phenyl]-5-methyl-pyrrol-1-yl}-benzoic acid
3-{2-[5-Trifluoromethyl-2-(2-chloro-4-fluoro-benzyloxy)-phenyl]-5-methyl-pyrrol-1-yl}-benzoic acid
3-[2-(5-Chloro-2-benzyloxy-phenyl)-5-methyl-pyrrol-1-yl]-N-(1-phenylsulfonyl)-benzamide
4-{2-[5-Chloro-2-(2,4-difluoro-benzyloxy)-phenyl]-5-methyl-pyrrol-1-yl}-N-(1-phenyl-methanoyl)-benzenesulfonamide
3-{2-[5-Chloro-2-(benzyloxy)-phenyl]-5-methyl-pyrrol-1yl}-N-[(S)-1-phenyl-ethyl]-benzamide
3-{2-[5-Bromo-2-(2,4-difluoro-benzyloxy)-phenyl]-5-methyl-pyrrol-1yl}-5-acetylamino-N-[(S)-1-phenyl-ethyl]-benzamide;
4-{2-[5-Bromo-2-(2,4-difluoro-benzyloxy-phenyl]-5-methyl-pyrrol-1-yl}-N-pyridin-2-yl-benzamide;
2-{2-[5-Chloro-2-(4-chloro-benzyloxy)-phenyl]-5-methyl-pyrrol-1-yl}-isonicotinic acid;
3-{2-[5-Bromo-2-(2,6-difluoro-benzyloxy)-phenyl]-5-methyl-pyrrol-1-yl}-benzoic acid;
3-{2-[5-Bromo-2-(2,4,6-trifluoro-benzyloxy)-phenyl]-5-methyl-pyrrol-1-yl}-benzoic acid;
3-{2-[5-Bromo-2-(2-fluoro-benzyloxy)-phenyl]-5-methyl-pyrrol-1-yl}-benzoic acid;
3-{2-[5-Bromo-2-(2,4-dichloro-benzyloxy)-phenyl]-5-methyl-pyrrol-1-yl}-benzoic acid;
3-{2-[5-Bromo-2-(benzyloxy)-phenyl]-5-methyl-pyrrol-1-yl}-6-acetylamino-benzoic acid;
3-{2-[5-Bromo-2-(benzyloxy)-phenyl]-5-methyl-pyrrol-1-yl}-5-(1,1-dioxo-1I⁶-isothiazolidin-2-yl)-benzoic acid;
3-{2-[5-Bromo-2-(benzyloxy)-phenyl]-5-methyl-pyrrol-1-yl}-5-amino-6-methyl-benzoic acid;
3-{2-[5-Bromo-2-(benzyloxy)-phenyl]-5-methyl-pyrrol-1-yl}-5-methyl-benzoic acid;
3-{2-[5-Bromo-2-(benzyloxy)-phenyl]-5-methyl-pyrrol-1-yl}-6-methoxy-benzoic acid;
3-{2-[5-Bromo-2-(2,4,6-trifluoro-benzyloxy)-phenyl]-5-methyl-pyrrol-1-yl}-5-amino-6-methyl-benzoic acid:
3-{2-[5-Bromo-2-(2,4,6-trifluoro-benzyloxy)-phenyl]-5-methyl-pyrrol-1-yl}-6-methyl-benzoic acid;
3-{2-[5-Bromo-2-(4-fluoro-benzyloxy)-phenyl]-5-methyl-pyrrol-1-yl}-6-fluoro-benzoic acid;
3-{2-[5-Bromo-2-(4-fluoro-benzyloxy)-phenyl]-5-methyl-pyrrol-1-yl}-6-hydroxy-benzoic acid;
3-{2-[5-Bromo-2-(4-fluoro-benzyloxy)-phenyl]-5-methyl-pyrrol-1-yl}-5-amino-6-methyl-benzoic acid;
3-{2-[5-Fluoro-2-(4-fluoro-benzyloxy)-phenyl]-5-methyl-pyrrol-1-yl}-6-methyl-benzoic acid;
3-{2-[5-Fluoro-2-(4-fluoro-benzyloxy)-phenyl]-5-methyl-pyrrol-1-yl}-5-amino-6-methyl-benzoic acid;
3-{2-[5-Fluoro-2-(2,4-difluoro-benzyloxy)-phenyl]-5-methyl-pyrrol-1-yl}-6-methyl-benzoic acid;
6-{2-[5-Trifluoromethyl-2-(benzyloxy)-phenyl]-5-methyl-pyrrol-1-yl}-picolinic acid;
6-{2-[5-Trifluoromethyl-2-(4-fluoro-benzyloxy)-phenyl]-5-methyl-pyrrol-1-yl}-picolinic-acid;
6-{2-[5-Trifluoromethyl-2-(2,6-difluoro-benzyloxy)-phenyl]-5-methyl-pyrrol-1-yl}-picolinic-acid:
6-{2-[5-Chloro-2-(4-fluoro-benzyloxy)-phenyl]-5-methyl-pyrrol-1-yl}-1-picolinic acid;
6-{2-[5-Bromo-2-(4-fluoro-benzyloxy)-phenyl]-5-methyl-pyrrol-1-yl}-picolinic acid;
3-{2-[5-Trifluoromethyl-2-(4-fluoro-benzyloxy)-phenyl]-5-methyl-pyrrol-1-yl}-6-methyl-benzoic acid methyl ester;
3-{2-[5-Trifluoromethyl-2-(4-fluoro-benzyloxy)-phenyl]-5-methyl-pyrrol-1-yl}-5-amino-6-methyl-benzoic acid methyl ester;
3-{2-[5-Trifluoromethyl-2-benzyloxy-phenyl]-5-methyl-pyrrol-1-yl}-5-amino-6-methyl-benzoic acid;
3-{2-[5-Trifluoromethyl-2-(2,4-difluoro-benzyloxy)-phenyl]-5-methyl-pyrrol-1-yl}-5-(methanesulfonyl)-benzoic acid;
4-{2-[5-Trifluoromethyl-2-(2,4:difluoro-benzyloxy)-phenyl]-5-methyl-pyrrol-1-yl}-2-methyl-benzoic acid;
3-{2-[5-Chloro-2-(2,6-difluoro-benzyloxy)-phenyl]-5-methyl-pyrrol-1-yl}-6-chloro-benzoic acid;
3-{2-[5-Chloro-2-(2,6-difluoro-benzyloxy)-phenyl]-5-methyl-pyrrol-1-yl}-6-methyl-benzoic acid;
3-{2-[5-Chloro-2-(2,3-difluoro-benzyloxy)-phenyl]-5-methyl-pyrrol-1-yl}-6-methyl-benzoic acid;
3-{2-[5-Bromo-2-(2,4-difluoro-benzyloxy)-phenyl]-5-methyl-pyrrol-1-yl}-5-methanesulfonylamino-benzoic acid;
3-{2-[5-Bromo-2-(2,4-difluoro-benzyloxy)-phenyl]-5-methyl-pyrrol-1-yl}-6-methoxy-benzoic acid;
3-{2-[5-Bromo-2-(2,4-difluoro-benzyloxy)-phenyl]-5-methyl-pyrrol-1-yl}-5-(1,1-dioxo-1I⁶-isothiazolidin-2-yl)-benzoic acid;
3-{2-[5-Methyl-2-(4-fluoro-benzyloxy)-phenyl]-5-methyl-pyrrol-1-yl}-6-difluoromethoxy-benzoic acid;
3-{2-[5-Methyl-2-(4-fluoro-benzyloxy)-phenyl]-5-methyl-pyrrol-1-yl}-5-amino-6-methyl-benzoic acid;
3-{2-[5-Chloro-2-(benzyloxy)-phenyl]-5-methyl-pyrrol-1-yl}-5-amino-6-methyl-benzoic acid;
3-{2-[5-Chloro-2-(benzyloxy)-phenyl]-5-methyl-pyrrol-1-yl}-5-methyl-benzoic acid;
6-{2-[5-Chloro-2-(benzyloxy)-phenyl]-5-methyl-pyrrol-1-yl}-1H-indole-4-carboxylic acid;
3-{2-[5-Chloro-2-(benzyloxy)-phenyl]-5-methyl-pyrrol-1-yl}-5-methoxycarbonylamino-benzoic acid;
4-{2-[5-Chloro-2-(benzyloxy)-phenyl]-5-methyl-pyrrol-1-yl}-N-(pyridin-2-ylmethyl)-benzamide;
3-{2-[5-Bromo-2-(2,4-difluoro-benzyloxy)-phenyl]-5-methyl-pyrrol-1-yl}-6-methyl-benzoic acid;
3-{2-[5-Bromo-2-(2,4-difluoro-benzyloxy)-phenyl)]-5-methypyrrol-1-yl}-6-difluoromethoxy-benzoic acid;
5-(3-{2-[5-Bromo-2-(2,4-difluoro-benzyloxy)-phenyl]-5-methyl-pyrrol-1-yl}-phenyl}-1H-tetrazole;
2-(4-{2-[5-Bromo-2-(2,4-difluoro-benzyloxy)-phenyl]-5-methyl-pyrrol-1yl}-phenyl)-1,1,1,3,3,3-hexafluoro-propan-2-ol;
5-(4-{2-[5-Bromo-2-(2,4,6-trifluoro-benzyloxy)-phenyl]-5-methyl-pyrrol-1-yl}-benzyl)-1H-tetrazole;
4-{2-[5-Chloro-2-(benzyloxy)-phenyl]-5-methyl-pyrrol-1-yl}-benzoic acid;
and pharmaceutically acceptable derivatives thereof.

7. A pharmaceutical composition comprising a compound according to claim 1 together with a pharmaceutical carrier and/or excipient.

8-9. (canceled)

10. A method of treating a human or animal subject suffering from a condition which is mediated by the action of PGE₂ at EP₁ receptors which comprises administering to said subject an effective amount of a compound according to claim 1.

11. A method of treating a human or animal subject suffering from a pain, inflammatory, immunological, bone, neurodegenerative or renal disorder, which method comprises administering to said subject an effective amount of a compound according to claim 1.

12. A method of treating a human or animal subject suffering from inflammatory pain, neuropathic pain or visceral pain which method comprises administering to said subject an effective amount of a compound according to claim 1.

13-16. (canceled)
